(19) United States
(12) Patent Application Publication
(10) Pub. No.: US 2024/0246987 A1
(43) Pub. Date: Jul. 25, 2024

(54) HETEROCYCLIC COMPOUND, ORGANIC LIGHT-EMITTING DEVICE COMPRISING SAME, AND COMPOSITION FOR ORGANIC LAYER

(71) Applicant: LT MATERIALS CO., LTD., Yongin-si, Gyeonggi-do (KR)

(72) Inventors: Cheol Hun JEONG, Yongin-si (KR); Hyun Joo LEE, Yongin-si (KR); Young Seok NO, Yongin-si (KR); Dong Jun KIM, Yongin-si (KR)

(73) Assignee: LT MATERIALS CO., LTD., Yongin-si, Gyeonggi-do (KR)

(21) Appl. No.: 18/285,401

(22) PCT Filed: Jun. 14, 2022
(86) PCT No.: PCT/KR2022/008370

§ 371 Date: Oct. 3, 2023

(30) Foreign Application Priority Data
Jul. 13, 2021 (KR) 10-2021-0091450

(51) Int. Cl. C07D 487/06; C07D 403/10; C09K 11/06; H10K 85/30; H10K 85/60

(52) U.S. Cl. CPC C07D 487/06; C07D 403/10; C09K 11/06; H10K 85/342; H10K 85/622; H10K 85/654; H10K 85/6572; H10K 85/6574; H10K 85/6576; C09K 2211/1033; C09K 2211/1044; C09K 2211/1092

(57) ABSTRACT

The present disclosure relates to a heterocyclic compound represented by Chemical Formula 1, an organic light emitting device including the same, and a composition for an organic material layer.

### TECHNICAL FIELD

This application claims priority to and the benefits of Korean Patent Application No. 10-2021-0091450, filed with the Korean Intellectual Property Office on Jul. 13, 2021, the entire contents of which are incorporated herein by reference.

The present disclosure relates to a heterocyclic compound, an organic light emitting device comprising the same, and a composition for an organic material layer.

### BACKGROUND ART

An organic light emitting device is one type of self-emissive display devices, and has advantages of having a wide viewing angle and a high response speed as well as having an excellent contrast.

An organic light emitting device has a structure of disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transport, electron blocking, hole blocking, electron transport, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

U.S. Pat. No. 4,356,429

### DISCLOSURE

### Technical Problem

An object of the present disclosure is to provide a heterocyclic compound, an organic light emitting device comprising the same, and a composition for an organic material layer.

### Technical Solution

In order to achieve the above object, the present disclosure provides a heterocyclic compound represented by the following Chemical Formula 1.

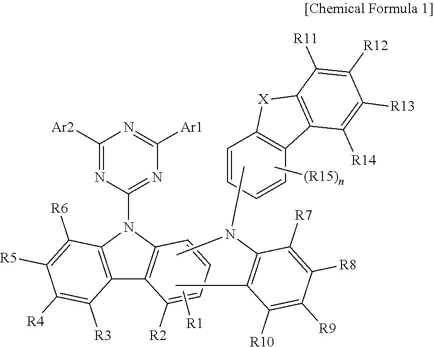

In Chemical Formula 1,
R1 to R15 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
Ar1 and Ar2 are the same as or different from each other, and each independently a substituted or unsubstituted C6 to C60 aryl group; or a substituted or unsubstituted C2 to C60 heteroaryl group,
X is S; O; CRaRb; or NRc,
n is an integer of 0 to 3, and when n is 2 or greater, R15s are the same as or different from each other,
Ra to Rc are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; and —NR201R202, or two or more groups adjacent to each other bond to each other to form a substituted or unsubstituted C6 to C60 aromatic hydrocarbon ring or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group, and
at least one of R11 to R15 is a substituted or unsubstituted C6 to C60 aryl group; or a substituted or unsubstituted C2 to C60 heteroaryl group.

In addition, the present disclosure provides an organic light emitting device comprising:
a first electrode;
a second electrode provided opposite to the first electrode; and
one or more organic material layers provided between the first electrode and the second electrode,
wherein at least one of the one or more layers of the organic material layers comprise the heterocyclic compound represented by Chemical Formula 1.

In addition, the present disclosure provides an organic light emitting device, wherein the organic material layer further comprises a heterocyclic compound represented by the following Chemical Formula 10.

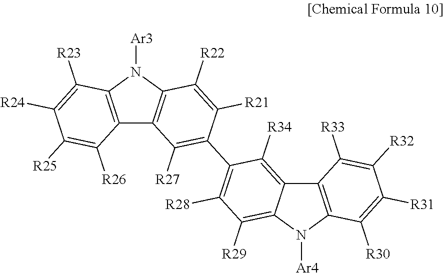

In Chemical Formula 10,
R21 to R34 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R301R302; —SiR301R302R303; and —NR301R302, or two or more groups adjacent to each other bond to each other to form a substituted or unsubstituted C6 to C60 aromatic hydrocarbon ring or a substituted or unsubstituted C2 to C60 heteroring, and R301, R302 and R303 are the same as or different from each other and each independently a substituted or unsubstituted C1 to C60 alkyl group; a substituted or unsubstituted C6 to C60 aryl group; or a substituted or unsubstituted C2 to C60 heteroaryl group, and
Ar3 and Ar4 are the same as or different from each other, and each independently a substituted or unsubstituted C6 to C60 aryl group; or a substituted or unsubstituted C2 to C60 heteroaryl group.

In addition, the present disclosure provides a composition for an organic material layer of an organic light emitting device, the composition comprising the heterocyclic compound represented by Chemical Formula 1 and the heterocyclic compound represented by Chemical Formula 10.

### Advantageous Effects

A compound described in the present specification can be used as a material of an organic material layer of an organic light emitting device. The compound is capable of performing a role of a hole injection layer material, an electron blocking layer material, a hole transport layer material, a light emitting layer material, an electron transport layer material, a hole blocking layer material, an electron injection layer material and the like in an organic light emitting device. Particularly, the compound can be used as a light emitting layer material of an organic light emitting device.

Specifically, the compound can be used as a light emitting material either alone or as a mixture with a P-type host, and can be used as a host material or a dopant material of a light emitting layer. Using the compound represented by Chemical Formula 1 in an organic material layer is capable of lowering a driving voltage, enhancing light emission efficiency and enhancing lifetime properties in an organic light emitting device.

More specifically, the heterocyclic compound represented by Chemical Formula 1 of the present disclosure is capable of effectively stabilizing electrons by increasing a delocalization rate of the HOMO site through expanding the resonance structure, and is thereby particularly capable of enhancing lifetime properties.

### DESCRIPTION OF DRAWINGS

FIG. 1 to FIG. 3 are diagrams each schematically illustrating a lamination structure of an organic light emitting device according to one embodiment of the present disclosure.

### MODE FOR DISCLOSURE

Hereinafter, the present disclosure will be described in more detail.

In the present specification, a term “substitution” means a hydrogen atom bonding to a carbon atom of a compound being changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, “substituted or unsubstituted” means being substituted with one or more substituents selected from the group consisting of deuterium; halogen; a cyano group; a C1 to C60 linear or branched alkyl group; a C2 to C60 linear or branched alkenyl group; a C2 to C60 linear or branched alkynyl group; a C3 to C60 monocyclic or polycyclic cycloalkyl group; a C2 to C60 monocyclic or polycyclic heterocycloalkyl group; a C6 to C60 monocyclic or polycyclic aryl group; a C2 to C60 monocyclic or polycyclic heteroaryl group; —SiRR′R″; —P(═O)RR′; a C1 to C20 alkylamine group; a C6 to C60 monocyclic or polycyclic arylamine group; and a C2 to C60 monocyclic or polycyclic heteroarylamine group or being unsubstituted, or being substituted with a substituent linking two or more substituents selected from among the substituents illustrated above.

In the present specification, the halogen may be fluorine, chlorine, bromine or iodine.

In the present specification, the alkyl group includes linear or branched having 1 to 60 carbon atoms, and may be further substituted with other substituents. The number of carbon atoms of the alkyl group may be from 1 to 60, specifically from 1 to and more specifically from 1 to 20. Specific examples thereof may include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 4-methylhexyl group, a 5-methylhexyl group and the like, but are not limited thereto.

In the present specification, the alkenyl group includes linear or branched having 2 to 60 carbon atoms, and may be further substituted with other substituents. The number of carbon atoms of the alkenyl group may be from 2 to 60, specifically from 2 to and more specifically from 2 to 20. Specific examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group includes linear or branched having 2 to 60 carbon atoms, and may be further substituted with other substituents. The number of carbon atoms of the alkynyl group may be from 2 to 60, specifically from 2 to and more specifically from 2 to 20.

In the present specification, the alkoxy group may be linear, branched or cyclic. The number of carbon atoms of the alkoxy group is not particularly limited, but is preferably from 1 to 20. Specific examples thereof may include a methoxy group, an ethoxy group, an n-propoxy group, an isopropoxy group, an n-butoxy group, an isobutoxy group, a tert-butoxy group, a sec-butoxy group, an n-pentyloxy group, a neopentyloxy group, an isopentyloxy group, an n-hexyloxy group, a 3,3-dimethylbutyloxy group, a 2-ethylbutyloxy group, an n-octyloxy group, an n-nonyloxy group, an n-decyloxy group, a benzyloxy group, a p-methylbenzyloxy group and the like, but are not limited thereto.

In the present specification, the cycloalkyl group includes monocyclic or polycyclic having 3 to 60 carbon atoms, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon groups of the cycloalkyl group may be from 3 to 60, specifically from 3 to 40 and more specifically from 5 to 20. Specific examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes monocyclic or polycyclic having 2 to 60 carbon atoms, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes monocyclic or polycyclic having 6 to 60 carbon atoms, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group may include a spiro group. The number of carbon atoms of the aryl group may be from 6 to 60, specifically from 6 to 40 and more specifically from 6 to 25. Specific examples of the aryl group may include a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring group thereof, and the like, but are not limited thereto.

In the present specification, the phosphine oxide group is represented by —P(═O)R101R102, and R101 and R102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group.

Specifically, the phosphine oxide group may be substituted with an aryl group, and as the aryl group, the examples described above may be used. Examples of the phosphine oxide group may include a diphenylphosphine oxide group, a dinaphthylphosphine oxide group and the like, but are not limited thereto.

In the present specification, the silyl group is a substituent including Si and having the Si atom directly linked as a radical, and is represented by —SiR101R102R103. R101 to R103 are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

When the fluorenyl group is substituted,

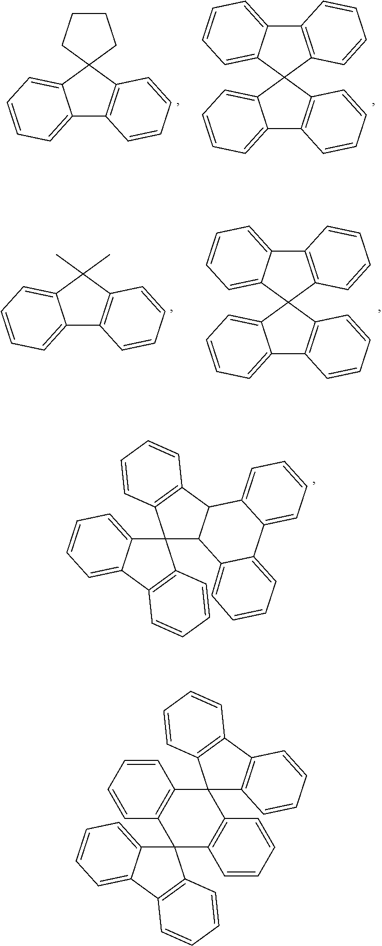

and the like may be included, however, the structure is not limited thereto.

In the present specification, the heteroaryl group includes S, O, Se, N or Si as a heteroatom, includes monocyclic or polycyclic having 2 to 60 carbon atoms, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may include a pyridyl group, a pyrrolyl group, a pyrimidyl group, a pyridazinyl group, a furanyl group, a thiophenyl group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, an imidazopyridinyl group, a diazanaphthalenyl group, a triazaindenyl group, a 2-indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl group, a benzocarbazolyl group, a dibenzocarbazolyl group, a phenazinyl group, a dibenzosilole group, a spirobi(dibenzosilole) group, a dihydrophenazinyl group, a phenoxazinyl group, a phenanthridyl group, a thienyl group, an indolo[2,3-a]carbazolyl group, an indolo[2,3-b]carbazolyl group, an indolinyl group, a 10,11-dihydro-dibenzo[b,f]azepinyl group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring may be interpreted as groups “adjacent” to each other.

In the present disclosure, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present disclosure, a “case of a substituent being not indicated in a chemical formula or compound structure” may mean that positions that may come as a substituent may all be hydrogen or deuterium. In other words, since deuterium is an isotope of hydrogen, some hydrogen atoms may be deuterium that is an isotope, and herein, a content of the deuterium may be from 0% to 100%.

In one embodiment of the present disclosure, in a “case of a substituent being not indicated in a chemical formula or compound structure”, hydrogen and deuterium may be mixed in compounds when deuterium is not explicitly excluded such as “a deuterium content being 0%”, “a hydrogen content being 100%” or “substituents being all hydrogen”.

In one embodiment of the present disclosure, deuterium is one of isotopes of hydrogen, is an element having deuteron formed with one proton and one neutron as a nucleus, and may be expressed as hydrogen-2, and the elemental symbol may also be written as D or ²H.

In one embodiment of the present disclosure, an isotope means an atom with the same atomic number (Z) but with a different mass number (A), and may also be interpreted as an element with the same number of protons but with a different number of neutrons.

In one embodiment of the present disclosure, a meaning of a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

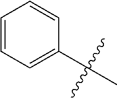 [SMILES: *c1ccccc1]

means that the total number of substituents that the phenyl group may have is 5 (T1 in the formula), and the number of deuterium among these is 1 (T2 in the formula). In other words, having a deuterium content of 20% in a phenyl group may be represented by the following structural formulae.

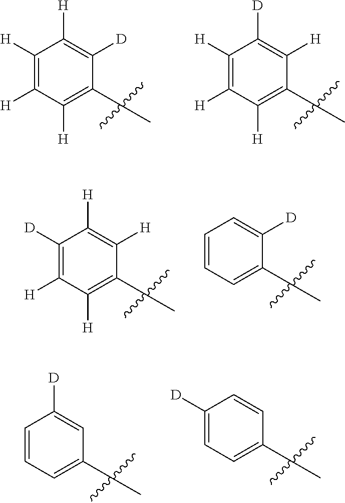 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present disclosure, “a phenyl group having a deuterium content of 0%” may mean a phenyl group that does not include a deuterium atom, that is, a phenyl group that has 5 hydrogen atoms.

In the present disclosure, the C6 to C60 aromatic hydrocarbon ring means a compound including an aromatic ring formed with C6 to C60 carbons and hydrogens. Examples thereof may include phenyl, biphenyl, terphenyl, triphenylene, naphthalene, anthracene, phenalene, phenanthrene, fluorene, pyrene, chrysene, perylene, azulene and the like, but are not limited thereto, and include all aromatic hydrocarbon ring compounds known in the art satisfying the above-mentioned number of carbon atoms.

The present disclosure provides a heterocyclic compound represented by the following Chemical Formula 1.

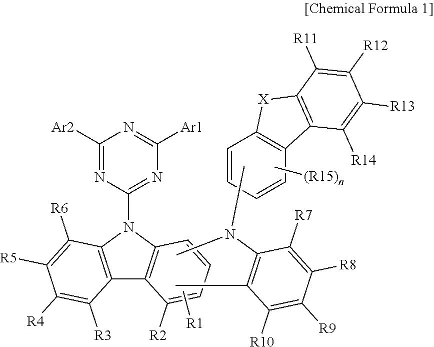

In Chemical Formula 1,
R1 to R15 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
Ar1 and Ar2 are the same as or different from each other, and each independently a substituted or unsubstituted C6 to C60 aryl group; or a substituted or unsubstituted C2 to C60 heteroaryl group,
X is S; O; CRaRb; or NRc,
n is an integer of 0 to 3, and when n is 2 or greater, R15s are the same as or different from each other,
Ra to Rc are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; and —NR201R202, or two or more groups adjacent to each other bond to each other to form a substituted or unsubstituted C6 to C60 aromatic hydrocarbon ring or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group, and
at least one of R11 to R15 is a substituted or unsubstituted C6 to C60 aryl group; or a substituted or unsubstituted C2 to C60 heteroaryl group.

In one embodiment of the present disclosure, R1 to R15 are the same as or different from each other, and may be each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102; —SiR101R102R103; or —NR101R102.

In another embodiment of the present disclosure, R1 to R15 are the same as or different from each other, and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; —SiR101R102R103; or —NR101R102.

In another embodiment of the present disclosure, R1 to R15 are the same as or different from each other, and may be each independently hydrogen; deuterium; a substituted or unsubstituted C6 to C20 aryl group; or a substituted or unsubstituted C2 to C20 heteroaryl group.

In another embodiment of the present disclosure, R1 to R15 are the same as or different from each other, and may be each independently hydrogen; deuterium; or a substituted or unsubstituted C6 to C20 aryl group.

In another embodiment of the present disclosure, R1 to R15 are the same as or different from each other, and may be each independently hydrogen; deuterium; a substituted or unsubstituted phenyl group; a substituted or unsubstituted biphenyl group; or a substituted or unsubstituted naphthyl group.

In one embodiment of the present disclosure, at least one of R11 to R15 may be a substituted or unsubstituted C6 to C30 aryl group; or a substituted or unsubstituted C2 to C30 heteroaryl group.

In another embodiment of the present disclosure, at least one of R11 to R15 may be a substituted or unsubstituted C6 to C20 aryl group; or a substituted or unsubstituted C2 to C20 heteroaryl group.

In another embodiment of the present disclosure, at least one of R11 to R15 may be a substituted or unsubstituted C6 to C20 aryl group.

In another embodiment of the present disclosure, at least one of R11 to R15 may be a substituted or unsubstituted phenyl group; a substituted or unsubstituted biphenyl group; or a substituted or unsubstituted naphthyl group.

In one embodiment of the present disclosure, when at least one of R11 to R14 is a substituted or unsubstituted C6 to C60 aryl group; or a substituted or unsubstituted C2 to C60 heteroaryl group, R15 may be hydrogen; or deuterium.

In another embodiment of the present disclosure, when at least one of R11 to R14 is a substituted or unsubstituted C6 to C30 aryl group; or a substituted or unsubstituted C2 to C30 heteroaryl group, R15 may be hydrogen; or deuterium.

In another embodiment of the present disclosure, when at least one of R11 to R14 is a substituted or unsubstituted C6 to C20 aryl group; or a substituted or unsubstituted C2 to C20 heteroaryl group, R15 may be hydrogen; or deuterium.

In another embodiment of the present disclosure, when at least one of R11 to R14 is a substituted or unsubstituted C6 to C20 aryl group, R15 may be hydrogen or deuterium.

In another embodiment of the present disclosure, when at least one of R11 to R14 is a substituted or unsubstituted phenyl group; a substituted or unsubstituted biphenyl group; or a substituted or unsubstituted naphthyl group, R15 may be hydrogen; or deuterium.

In one embodiment of the present disclosure, when R11 to R14 are the same as or different from each other and each independently hydrogen; or deuterium, n is 1 or greater, and at least one of R15s may be a substituted or unsubstituted C6 to C60 aryl group; or a substituted or unsubstituted C2 to C60 heteroaryl group.

In another embodiment of the present disclosure, when R11 to R14 are the same as or different from each other and each independently hydrogen; or deuterium, n is 1 or greater, and at least one of R15s may be a substituted or unsubstituted C6 to C30 aryl group; or a substituted or unsubstituted C2 to C30 heteroaryl group.

In another embodiment of the present disclosure, when R11 to R14 are the same as or different from each other and each independently hydrogen; or deuterium, n is 1 or greater, and at least one of R15s may be a substituted or unsubstituted C6 to C20 aryl group; or a substituted or unsubstituted C2 to C20 heteroaryl group.

In another embodiment of the present disclosure, when R11 to R14 are the same as or different from each other and each independently hydrogen; or deuterium, n is 1 or greater, and at least one of R15s may be a substituted or unsubstituted C6 to C20 aryl group.

In another embodiment of the present disclosure, when R11 to R14 are the same as or different from each other and each independently hydrogen; or deuterium, n is 1 or greater, and at least one of R15s may be a substituted or unsubstituted phenyl group; a substituted or unsubstituted biphenyl group; or a substituted or unsubstituted naphthyl group.

In one embodiment of the present disclosure, Ar1 and Ar2 are the same as or different from each other, and may be each independently a substituted or unsubstituted C6 to C30 aryl group; or a substituted or unsubstituted C2 to C30 heteroaryl group.

In another embodiment of the present disclosure, Ar and Ar2 are the same as or different from each other, and may be each independently a substituted or unsubstituted C6 to C20 aryl group; or a substituted or unsubstituted C2 to C20 heteroaryl group.

In another embodiment of the present disclosure, Ar and Ar2 are the same as or different from each other, and may be each independently a substituted or unsubstituted phenyl group; a substituted or unsubstituted biphenyl group; a substituted or unsubstituted fluorenyl group; a substituted or unsubstituted triphenylenyl group; a substituted or unsubstituted dibenzofuranyl group; or a substituted or unsubstituted dibenzothiophenyl group.

In one embodiment of the present disclosure, Ra to Rc are the same as or different from each other, and may be each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R201R202; —SiR201R202R203; or —NR201R202.

In another embodiment of the present disclosure, Ra to Rc are the same as or different from each other, and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R201R202; —SiR201R202R203; or —NR201R202.

In another embodiment of the present disclosure, Ra to Rc are the same as or different from each other, and may be each independently hydrogen; deuterium; a substituted or unsubstituted C1 to C20 alkyl group; a substituted or unsubstituted C6 to C20 aryl group; or a substituted or unsubstituted C2 to C20 heteroaryl group.

In one embodiment of the present disclosure, Ra and Rb are the same as or different from each other, and may be each independently hydrogen; deuterium; or a substituted or unsubstituted C1 to C30 alkyl group.

In another embodiment of the present disclosure, Ra and Rb are the same as or different from each other, and may be each independently hydrogen; deuterium; or a substituted or unsubstituted C1 to C20 alkyl group.

In another embodiment of the present disclosure, Ra and Rb are the same as or different from each other, and may be each independently hydrogen; deuterium; or a substituted or unsubstituted methyl group.

In one embodiment of the present disclosure, Rc may be hydrogen; deuterium; a substituted or unsubstituted C6 to C30 aryl group; or a substituted or unsubstituted C2 to C30 heteroaryl group.

In another embodiment of the present disclosure, Rc may be hydrogen; deuterium; a substituted or unsubstituted C6 to C20 aryl group; or a substituted or unsubstituted C2 to C20 heteroaryl group.

In another embodiment of the present disclosure, Rc may be hydrogen; deuterium; or a substituted or unsubstituted C6 to C20 aryl group.

In another embodiment of the present disclosure, Rc may be hydrogen; deuterium; or a substituted or unsubstituted phenyl group.

In another embodiment of the present disclosure, Rc may be a substituted or unsubstituted phenyl group.

In one embodiment of the present disclosure, the compound represented by Chemical Formula 1 may not include deuterium as a substituent, or a content of deuterium may be greater than 0%, 1% or greater, 10% or greater, 20% or greater, 30% or greater, 40% or greater or 50% or greater, and may be 100% or less, 90% or less, 80% or less, 70% or less or 60% or less based on the total number of hydrogen atoms and deuterium atoms in Chemical Formula 1.

In another embodiment of the present disclosure, the compound represented by Chemical Formula 1 may not include deuterium as a substituent, or a content of deuterium may be from 1% to 100% based on the total number of hydrogen atoms and deuterium atoms.

In another embodiment of the present disclosure, the content of deuterium may be from 20% to 90% based on the total number of hydrogen atoms and deuterium atoms in the compound represented by Chemical Formula 1.

In another embodiment of the present disclosure, the content of deuterium may be from 30% to 80% based on the total number of hydrogen atoms and deuterium atoms in the compound represented by Chemical Formula 1.

In another embodiment of the present disclosure, the content of deuterium may be from 50% to 70% based on the total number of hydrogen atoms and deuterium atoms in the compound represented by Chemical Formula 1.

In one embodiment of the present disclosure, Chemical Formula 1 may be a heterocyclic compound represented by any one of the following Chemical Formulae 2 to 5.

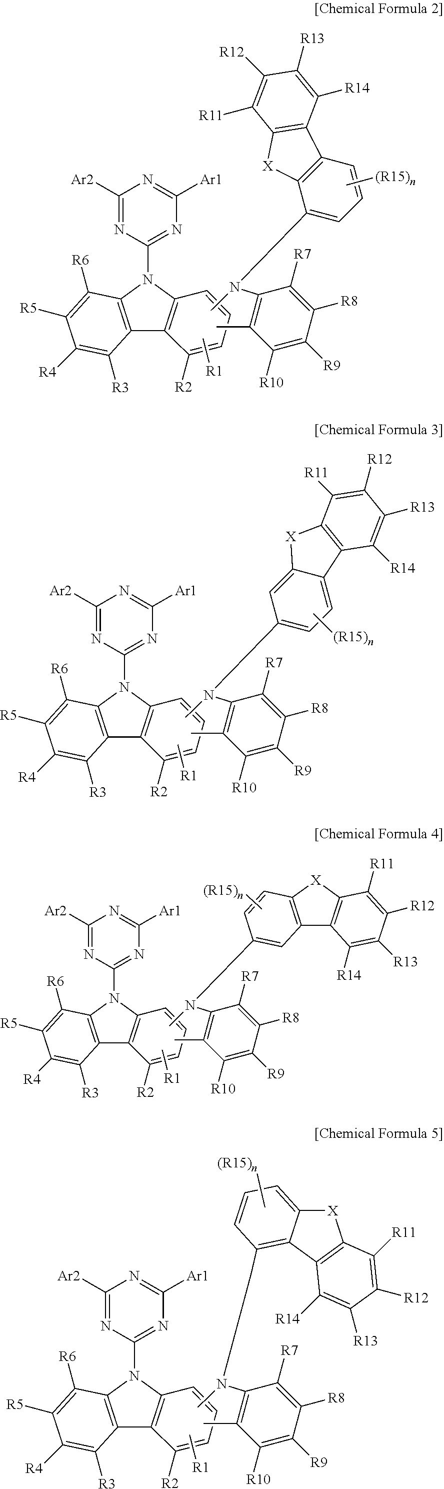

In Chemical Formulae 2 to 5,
R1 to R15, Ar, Ar2, X and n have the same definitions as in Chemical Formula 1.

In addition, in one embodiment of the present disclosure, Chemical Formula 1 may be a heterocyclic compound represented by any one of the following Chemical Formulae 6 to 9.

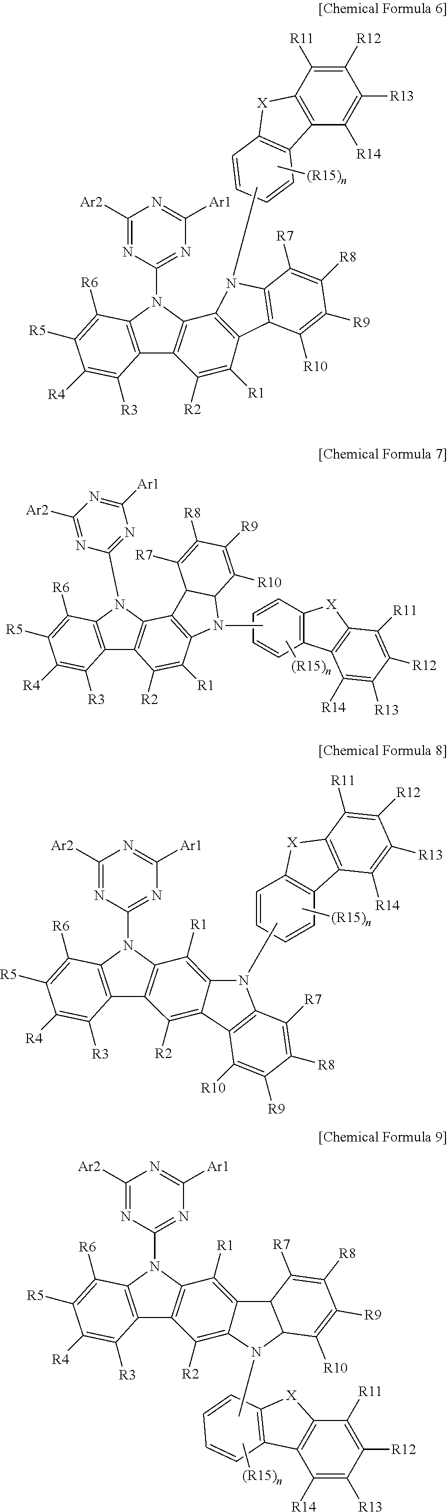

In Chemical Formulae 6 to 9,
R1 to R15, Ar1, Ar2, X and n have the same definitions as in Chemical Formula 1.

In one embodiment of the present disclosure, Chemical Formula 1 may be a heterocyclic compound represented by any one of the following compounds.

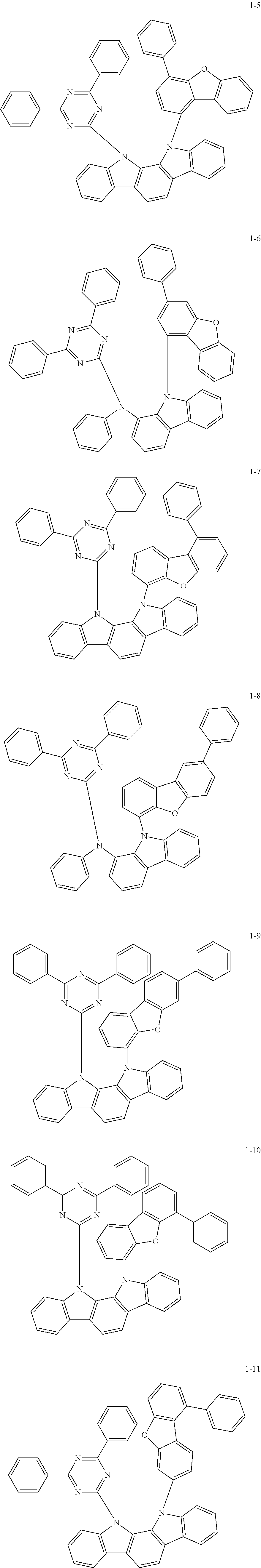

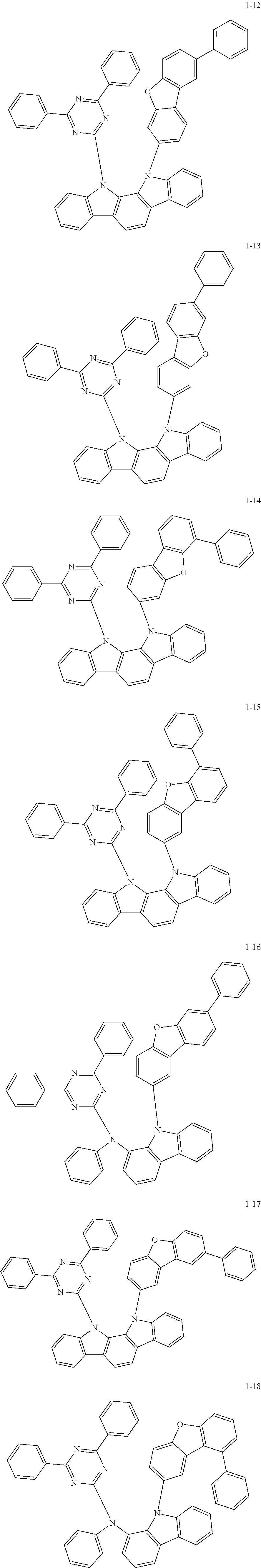

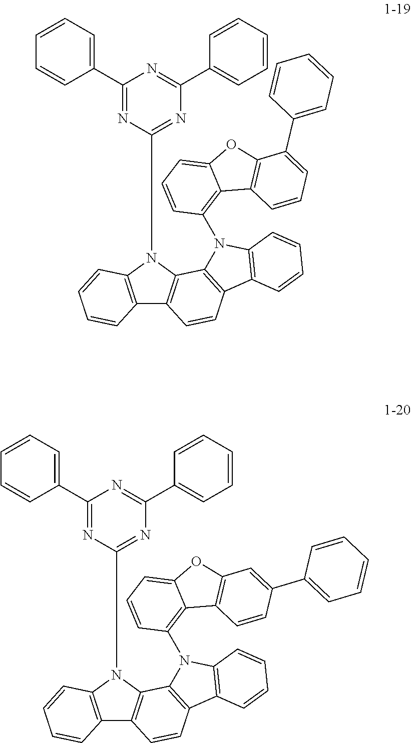 [SMILES: c1ccc(-c2nc(-c3ccccc3)nc(-n3c4ccccc4c4ccc5c6ccccc6n(-c6cccc7oc8c(-c9ccccc9)cccc8c67)c5c43)n2)cc1 ; c1ccc(-c2ccc3c(c2)oc2cccc(-n4c5ccccc5c5ccc6c7ccccc7n(-c7nc(-c8ccccc8)nc(-c8ccccc8)n7)c6c54)c23)cc1]

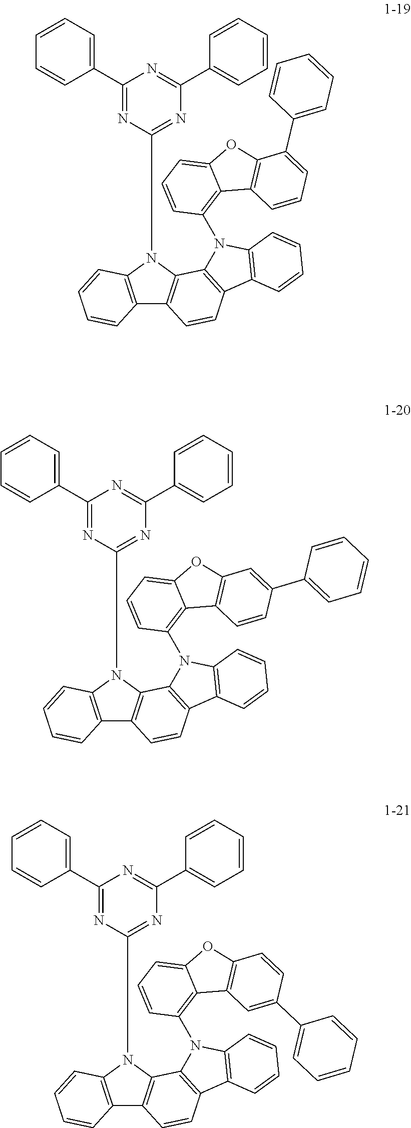 [SMILES: c1ccc(-c2nc(-c3ccccc3)nc(-n3c4ccccc4c4ccc5c6ccccc6n(-c6cccc7oc8c(-c9ccccc9)cccc8c67)c5c43)n2)cc1 ; c1ccc(-c2ccc3c(c2)oc2cccc(-n4c5ccccc5c5ccc6c7ccccc7n(-c7nc(-c8ccccc8)nc(-c8ccccc8)n7)c6c54)c23)cc1 ; c1ccc(-c2ccc3oc4cccc(-n5c6ccccc6c6ccc7c8ccccc8n(-c8nc(-c9ccccc9)nc(-c9ccccc9)n8)c7c65)c4c3c2)cc1]

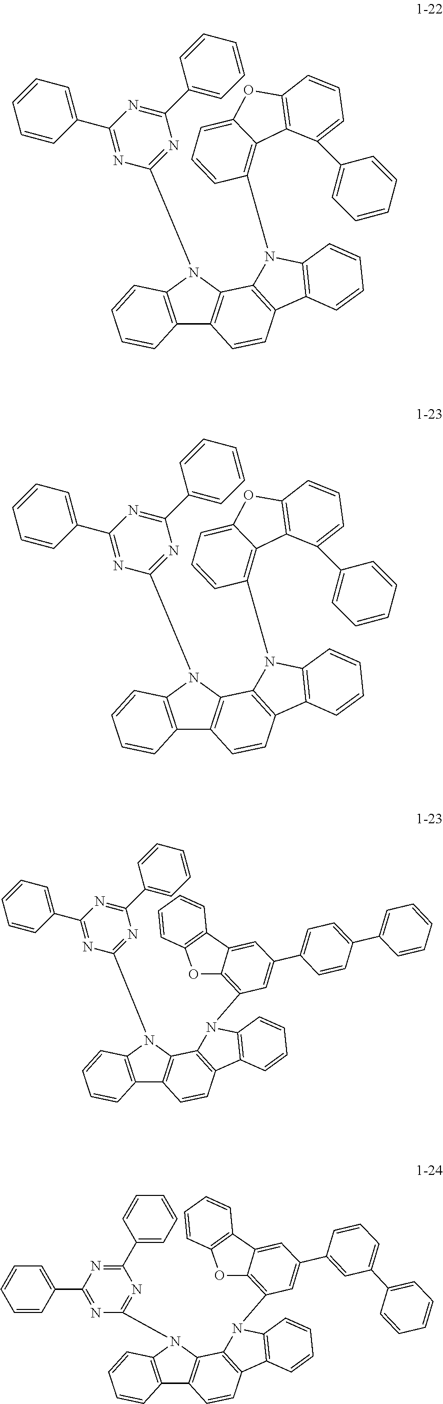 [SMILES: c1ccc(-c2nc(-c3ccccc3)nc(-n3c4ccccc4c4ccc5c6ccccc6n(-c6cccc7oc8cccc(-c9ccccc9)c8c67)c5c43)n2)cc1 ; c1ccc(-c2nc(-c3ccccc3)nc(-n3c4ccccc4c4ccc5c6ccccc6n(-c6cccc7oc8cccc(-c9ccccc9)c8c67)c5c43)n2)cc1 ; c1ccc(-c2ccc(-c3cc(-n4c5ccccc5c5ccc6c7ccccc7n(-c7nc(-c8ccccc8)nc(-c8ccccc8)n7)c6c54)c4oc5ccccc5c4c3)cc2)cc1 ; c1ccc(-c2cccc(-c3cc(-n4c5ccccc5c5ccc6c7ccccc7n(-c7nc(-c8ccccc8)nc(-c8ccccc8)n7)c6c54)c4oc5ccccc5c4c3)c2)cc1]

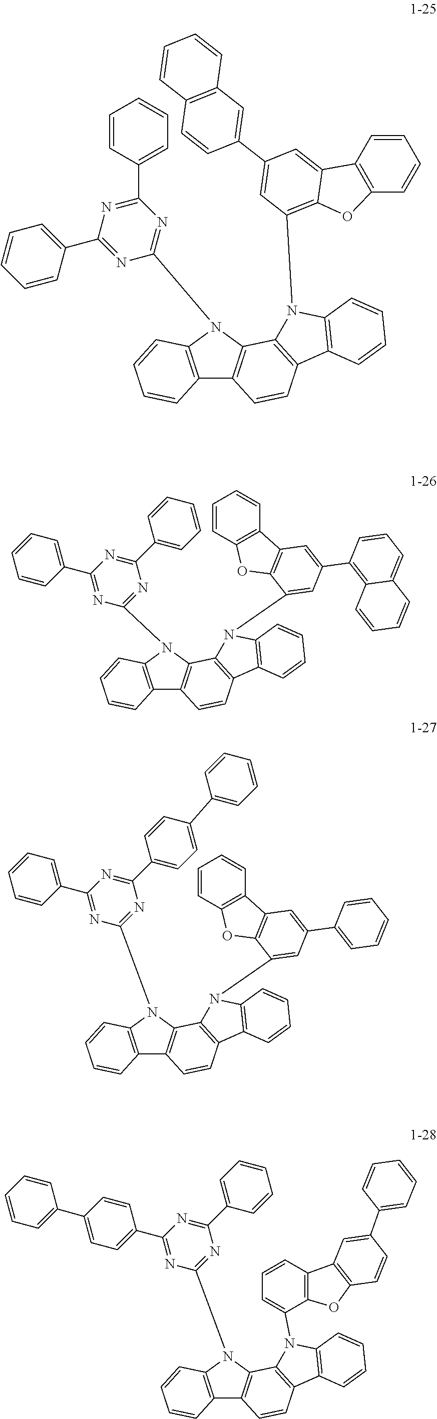 [SMILES: c1ccc(-c2nc(-c3ccccc3)nc(-n3c4ccccc4c4ccc5c6ccccc6n(-c6cc(-c7ccc8ccccc8c7)cc7c6oc6ccccc67)c5c43)n2)cc1 ; c1ccc(-c2nc(-c3ccccc3)nc(-n3c4ccccc4c4ccc5c6ccccc6n(-c6cc(-c7cccc8ccccc78)cc7c6oc6ccccc67)c5c43)n2)cc1 ; c1ccc(-c2ccc(-c3nc(-c4ccccc4)nc(-n4c5ccccc5c5ccc6c7ccccc7n(-c7cc(-c8ccccc8)cc8c7oc7ccccc78)c6c54)n3)cc2)cc1 ; c1ccc(-c2ccc(-c3nc(-c4ccccc4)nc(-n4c5ccccc5c5ccc6c7ccccc7n(-c7cccc8c7oc7ccc(-c9ccccc9)cc78)c6c54)n3)cc2)cc1]

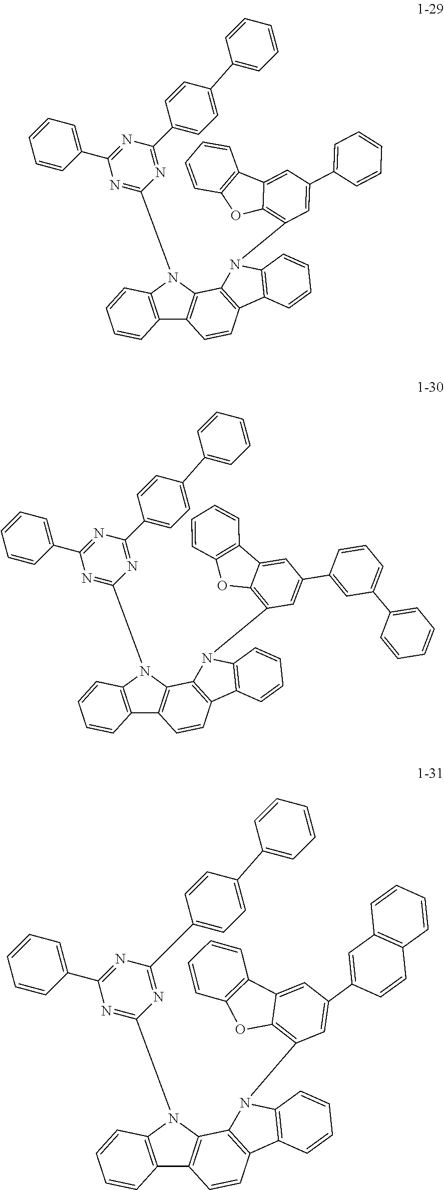 [SMILES: c1ccc(-c2ccc(-c3nc(-c4ccccc4)nc(-n4c5ccccc5c5ccc6c7ccccc7n(-c7cc(-c8ccccc8)cc8c7oc7ccccc78)c6c54)n3)cc2)cc1 ; c1ccc(-c2ccc(-c3nc(-c4ccccc4)nc(-n4c5ccccc5c5ccc6c7ccccc7n(-c7cc(-c8cccc(-c9ccccc9)c8)cc8c7oc7ccccc78)c6c54)n3)cc2)cc1 ; c1ccc(-c2ccc(-c3nc(-c4ccccc4)nc(-n4c5ccccc5c5ccc6c7ccccc7n(-c7cc(-c8ccc9ccccc9c8)cc8c7oc7ccccc78)c6c54)n3)cc2)cc1]

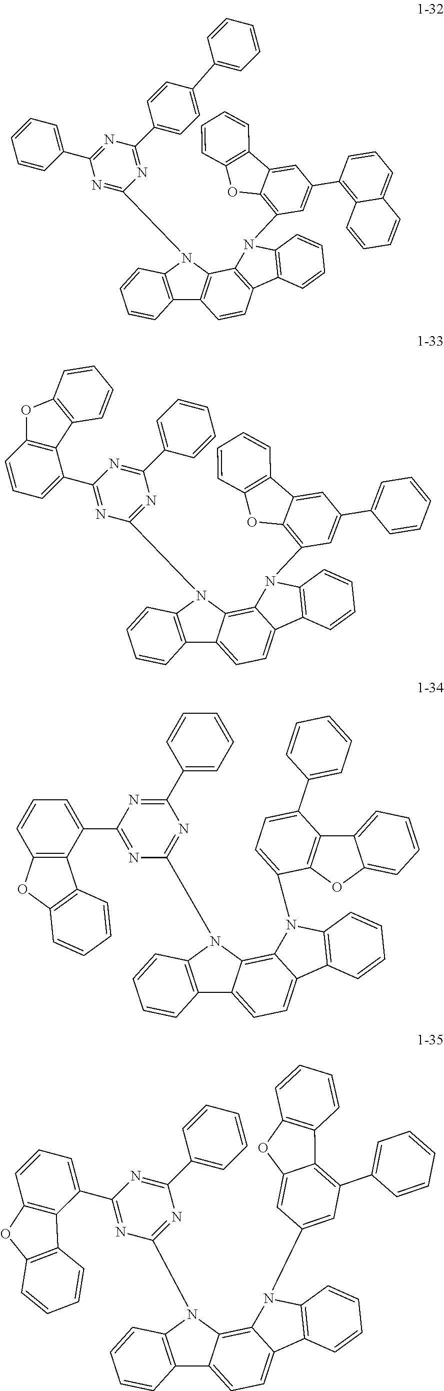 [SMILES: c1ccc(-c2ccc(-c3nc(-c4ccccc4)nc(-n4c5ccccc5c5ccc6c7ccccc7n(-c7cc(-c8cccc9ccccc89)cc8c7oc7ccccc78)c6c54)n3)cc2)cc1 ; c1ccc(-c2cc(-n3c4ccccc4c4ccc5c6ccccc6n(-c6nc(-c7ccccc7)nc(-c7cccc8oc9ccccc9c78)n6)c5c43)c3oc4ccccc4c3c2)cc1 ; c1ccc(-c2nc(-c3cccc4oc5ccccc5c34)nc(-n3c4ccccc4c4ccc5c6ccccc6n(-c6ccc(-c7ccccc7)c7c6oc6ccccc67)c5c43)n2)cc1 ; c1ccc(-c2nc(-c3cccc4oc5ccccc5c34)nc(-n3c4ccccc4c4ccc5c6ccccc6n(-c6cc(-c7ccccc7)c7c(c6)oc6ccccc67)c5c43)n2)cc1]

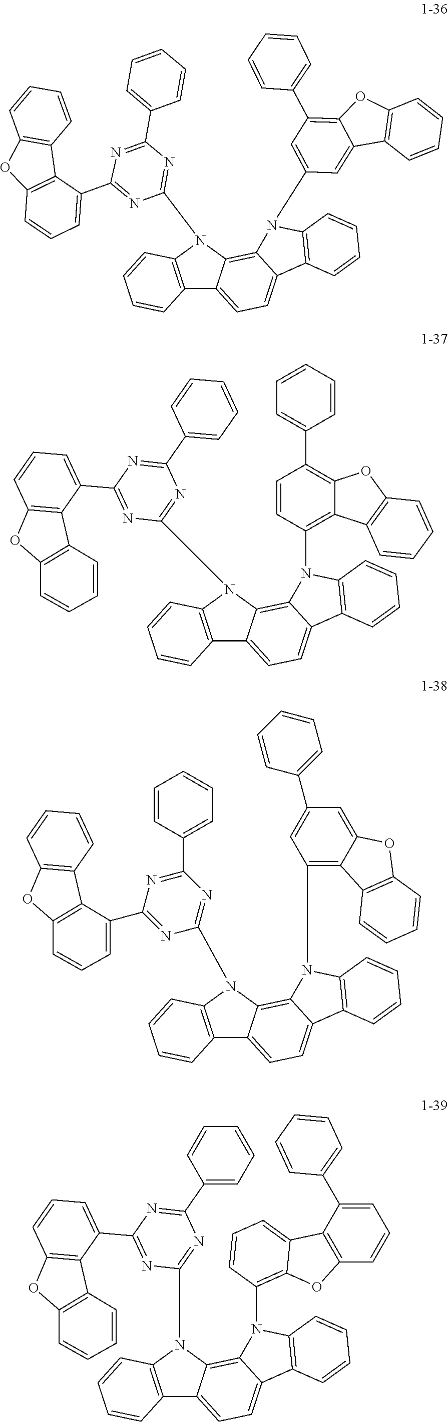 [SMILES: c1ccc(-c2nc(-c3cccc4oc5ccccc5c34)nc(-n3c4ccccc4c4ccc5c6ccccc6n(-c6cc(-c7ccccc7)c7oc8ccccc8c7c6)c5c43)n2)cc1 ; c1ccc(-c2nc(-c3cccc4oc5ccccc5c34)nc(-n3c4ccccc4c4ccc5c6ccccc6n(-c6ccc(-c7ccccc7)c7oc8ccccc8c67)c5c43)n2)cc1 ; c1ccc(-c2cc(-n3c4ccccc4c4ccc5c6ccccc6n(-c6nc(-c7ccccc7)nc(-c7cccc8oc9ccccc9c78)n6)c5c43)c3c(c2)oc2ccccc23)cc1 ; c1ccc(-c2nc(-c3cccc4oc5ccccc5c34)nc(-n3c4ccccc4c4ccc5c6ccccc6n(-c6cccc7c6oc6cccc(-c8ccccc8)c67)c5c43)n2)cc1]

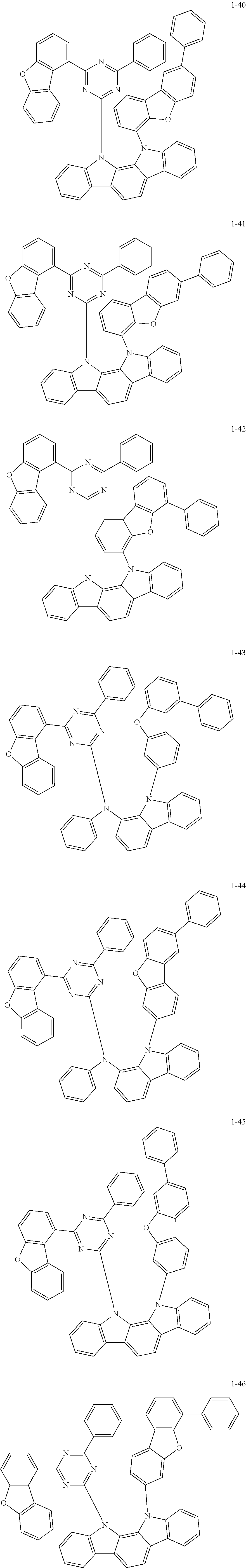

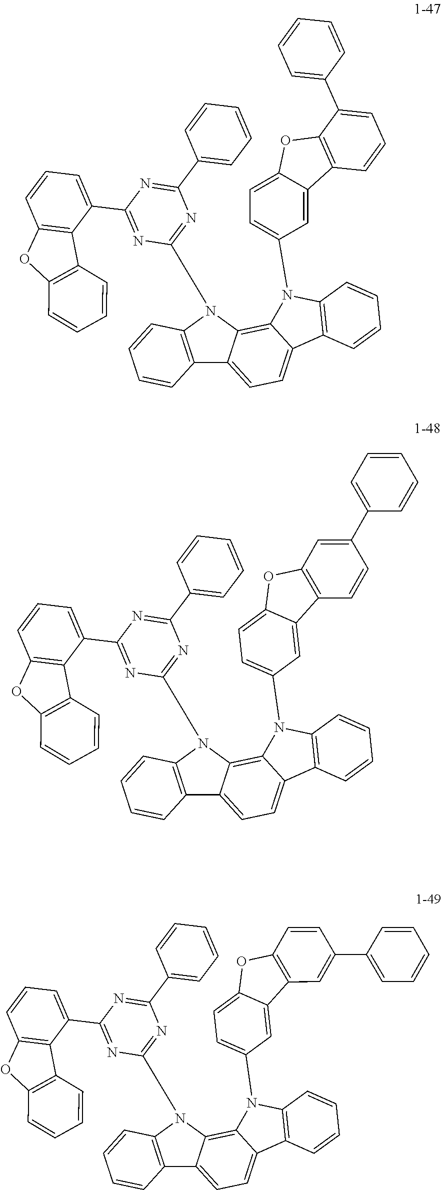 [SMILES: c1ccc(-c2nc(-c3cccc4oc5ccccc5c34)nc(-n3c4ccccc4c4ccc5c6ccccc6n(-c6ccc7oc8c(-c9ccccc9)cccc8c7c6)c5c43)n2)cc1 ; c1ccc(-c2ccc3c(c2)oc2ccc(-n4c5ccccc5c5ccc6c7ccccc7n(-c7nc(-c8ccccc8)nc(-c8cccc9oc%10ccccc%10c89)n7)c6c54)cc23)cc1 ; c1ccc(-c2ccc3oc4ccc(-n5c6ccccc6c6ccc7c8ccccc8n(-c8nc(-c9ccccc9)nc(-c9cccc%10oc%11ccccc%11c9%10)n8)c7c65)cc4c3c2)cc1]

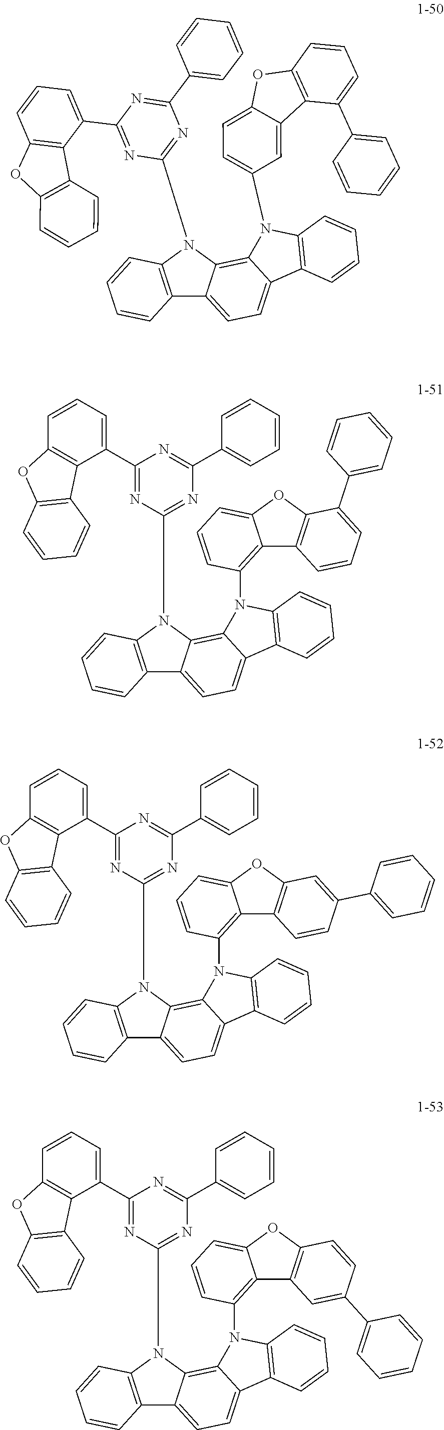 [SMILES: c1ccc(-c2nc(-c3cccc4oc5ccccc5c34)nc(-n3c4ccccc4c4ccc5c6ccccc6n(-c6ccc7oc8cccc(-c9ccccc9)c8c7c6)c5c43)n2)cc1 ; c1ccc(-c2nc(-c3cccc4oc5ccccc5c34)nc(-n3c4ccccc4c4ccc5c6ccccc6n(-c6cccc7oc8c(-c9ccccc9)cccc8c67)c5c43)n2)cc1 ; c1ccc(-c2ccc3c(c2)oc2cccc(-n4c5ccccc5c5ccc6c7ccccc7n(-c7nc(-c8ccccc8)nc(-c8cccc9oc%10ccccc%10c89)n7)c6c54)c23)cc1 ; c1ccc(-c2ccc3oc4cccc(-n5c6ccccc6c6ccc7c8ccccc8n(-c8nc(-c9ccccc9)nc(-c9cccc%10oc%11ccccc%11c9%10)n8)c7c65)c4c3c2)cc1]

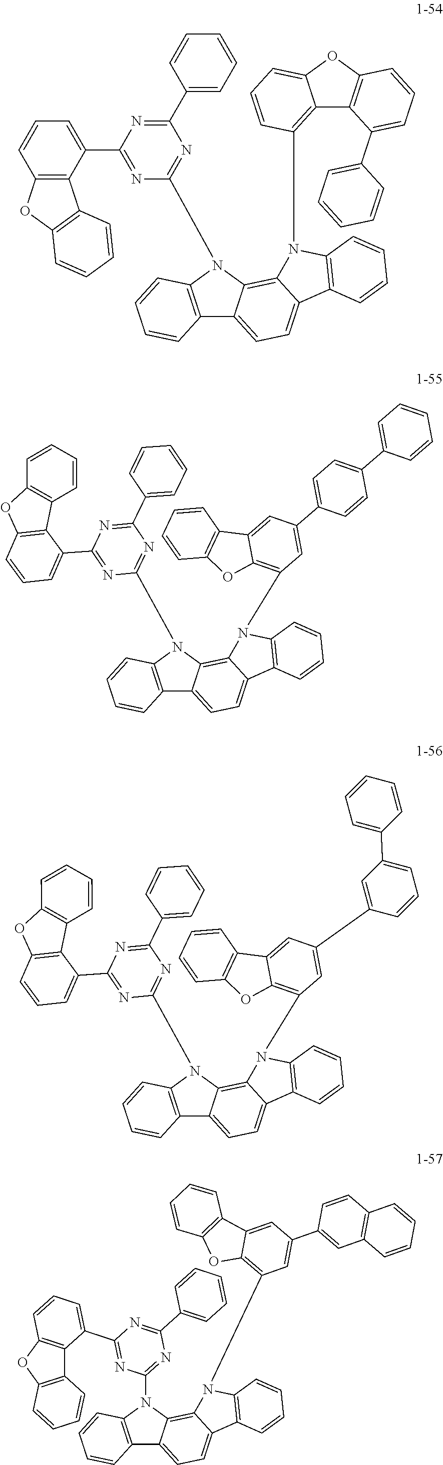 [SMILES: c1ccc(-c2nc(-c3cccc4oc5ccccc5c34)nc(-n3c4ccccc4c4ccc5c6ccccc6n(-c6cccc7oc8cccc(-c9ccccc9)c8c67)c5c43)n2)cc1 ; c1ccc(-c2ccc(-c3cc(-n4c5ccccc5c5ccc6c7ccccc7n(-c7nc(-c8ccccc8)nc(-c8cccc9oc%10ccccc%10c89)n7)c6c54)c4oc5ccccc5c4c3)cc2)cc1 ; c1ccc(-c2cccc(-c3cc(-n4c5ccccc5c5ccc6c7ccccc7n(-c7nc(-c8ccccc8)nc(-c8cccc9oc%10ccccc%10c89)n7)c6c54)c4oc5ccccc5c4c3)c2)cc1 ; c1ccc(-c2nc(-c3cccc4oc5ccccc5c34)nc(-n3c4ccccc4c4ccc5c6ccccc6n(-c6cc(-c7ccc8ccccc8c7)cc7c6oc6ccccc67)c5c43)n2)cc1]

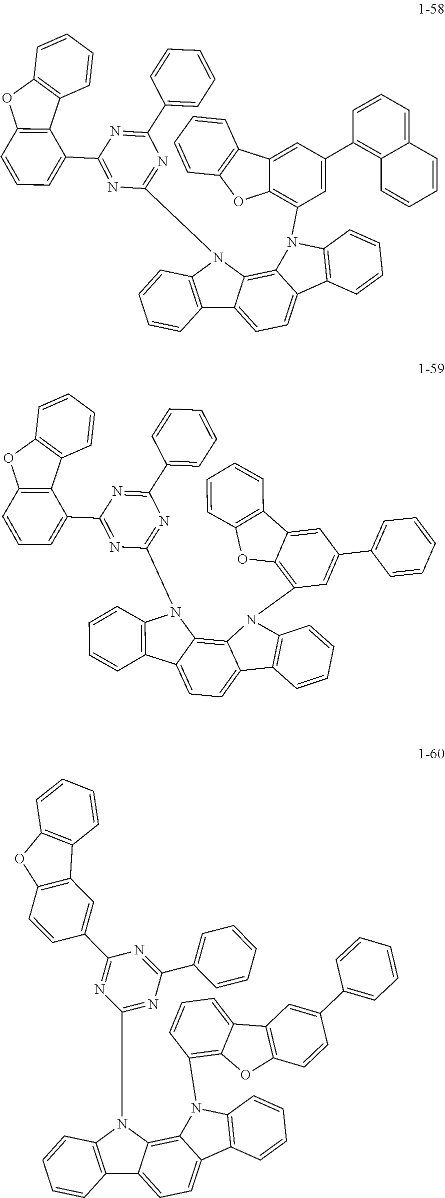 [SMILES: c1ccc(-c2nc(-c3cccc4oc5ccccc5c34)nc(-n3c4ccccc4c4ccc5c6ccccc6n(-c6cc(-c7cccc8ccccc78)cc7c6oc6ccccc67)c5c43)n2)cc1 ; c1ccc(-c2cc(-n3c4ccccc4c4ccc5c6ccccc6n(-c6nc(-c7ccccc7)nc(-c7cccc8oc9ccccc9c78)n6)c5c43)c3oc4ccccc4c3c2)cc1 ; c1ccc(-c2ccc3oc4c(-n5c6ccccc6c6ccc7c8ccccc8n(-c8nc(-c9ccccc9)nc(-c9ccc%10oc%11ccccc%11c%10c9)n8)c7c65)cccc4c3c2)cc1]

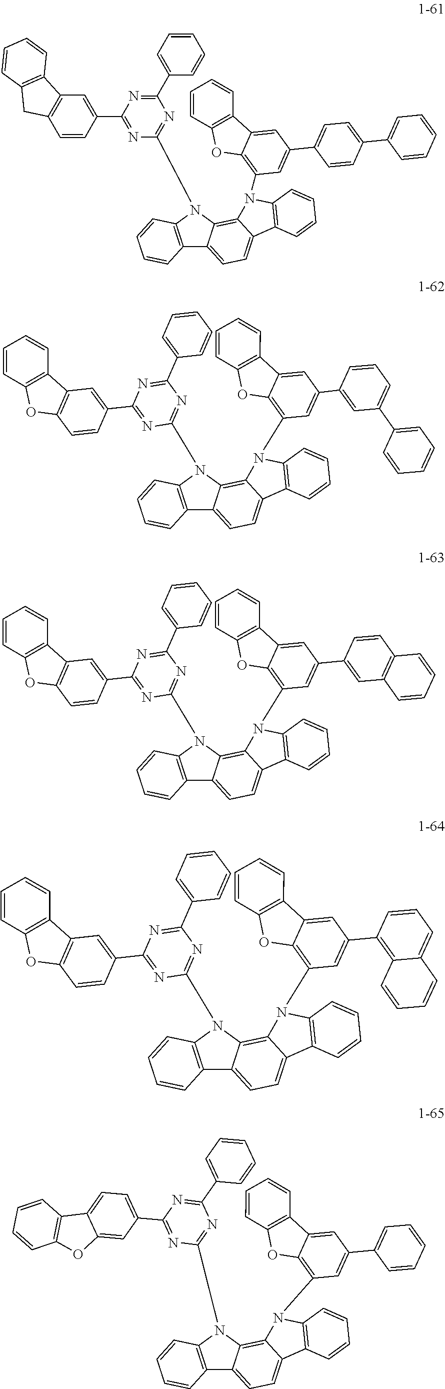

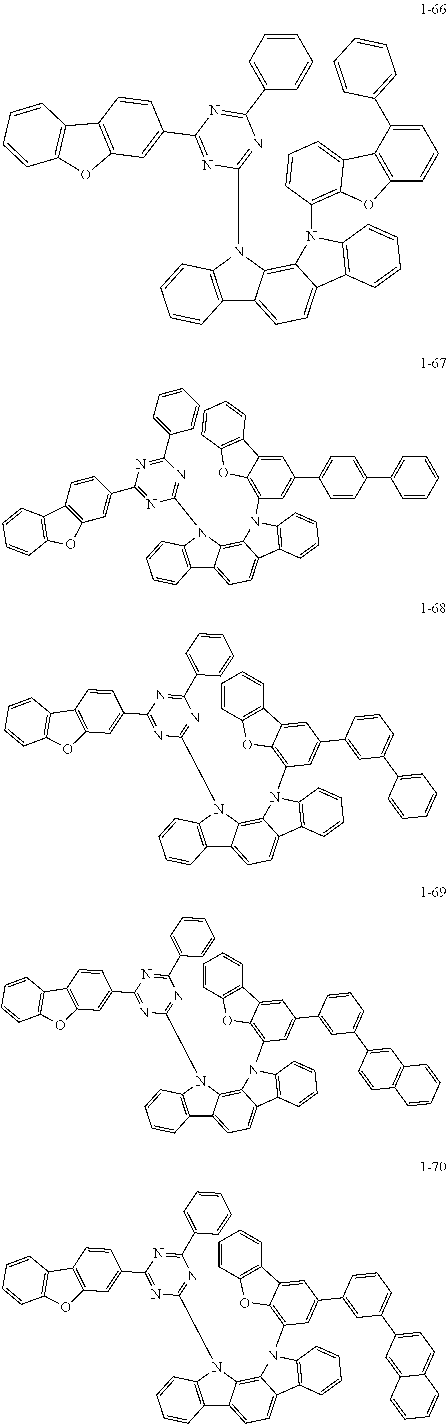

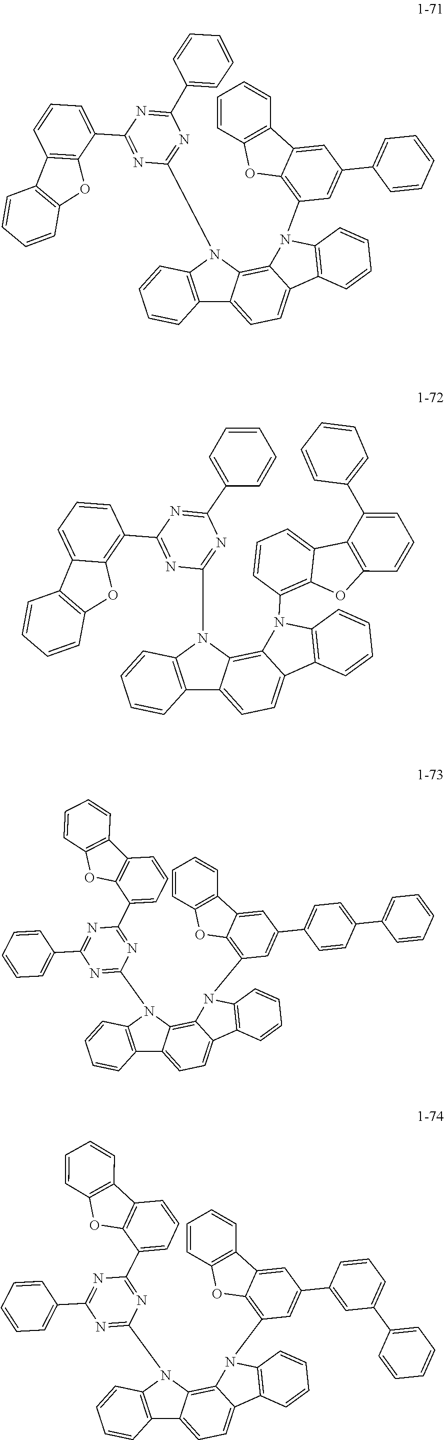 [SMILES: c1ccc(-c2cc(-n3c4ccccc4c4ccc5c6ccccc6n(-c6nc(-c7ccccc7)nc(-c7cccc8c7oc7ccccc78)n6)c5c43)c3oc4ccccc4c3c2)cc1 ; c1ccc(-c2nc(-c3cccc4c3oc3ccccc34)nc(-n3c4ccccc4c4ccc5c6ccccc6n(-c6cccc7c6oc6cccc(-c8ccccc8)c67)c5c43)n2)cc1 ; c1ccc(-c2ccc(-c3cc(-n4c5ccccc5c5ccc6c7ccccc7n(-c7nc(-c8ccccc8)nc(-c8cccc9c8oc8ccccc89)n7)c6c54)c4oc5ccccc5c4c3)cc2)cc1 ; c1ccc(-c2cccc(-c3cc(-n4c5ccccc5c5ccc6c7ccccc7n(-c7nc(-c8ccccc8)nc(-c8cccc9c8oc8ccccc89)n7)c6c54)c4oc5ccccc5c4c3)c2)cc1]

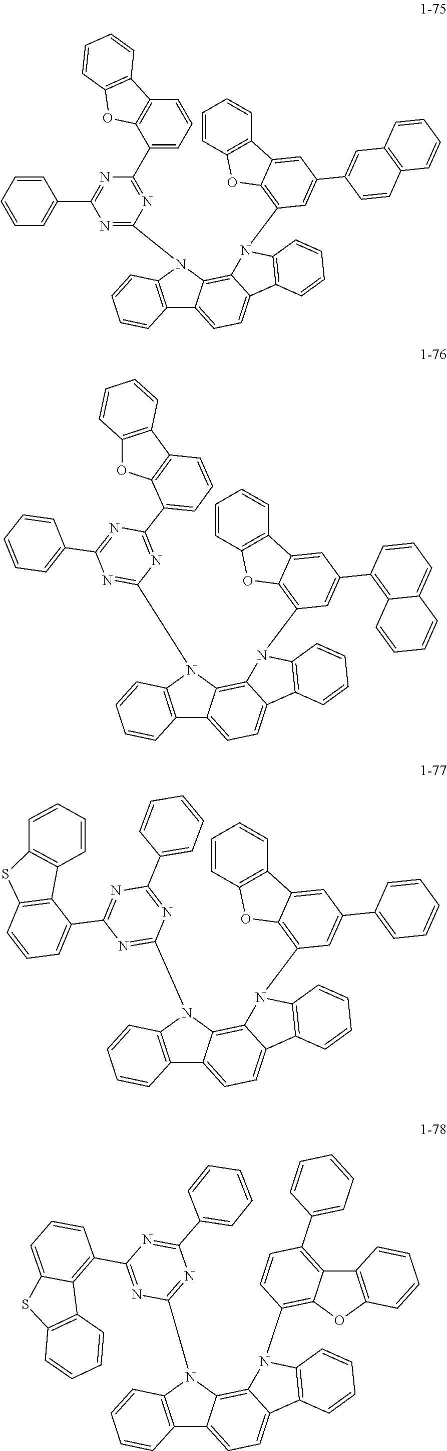 [SMILES: c1ccc(-c2nc(-c3cccc4c3oc3ccccc34)nc(-n3c4ccccc4c4ccc5c6ccccc6n(-c6cc(-c7ccc8ccccc8c7)cc7c6oc6ccccc67)c5c43)n2)cc1 ; c1ccc(-c2nc(-c3cccc4c3oc3ccccc34)nc(-n3c4ccccc4c4ccc5c6ccccc6n(-c6cc(-c7cccc8ccccc78)cc7c6oc6ccccc67)c5c43)n2)cc1 ; c1ccc(-c2cc(-n3c4ccccc4c4ccc5c6ccccc6n(-c6nc(-c7ccccc7)nc(-c7cccc8sc9ccccc9c78)n6)c5c43)c3oc4ccccc4c3c2)cc1 ; c1ccc(-c2nc(-c3cccc4sc5ccccc5c34)nc(-n3c4ccccc4c4ccc5c6ccccc6n(-c6ccc(-c7ccccc7)c7c6oc6ccccc67)c5c43)n2)cc1]

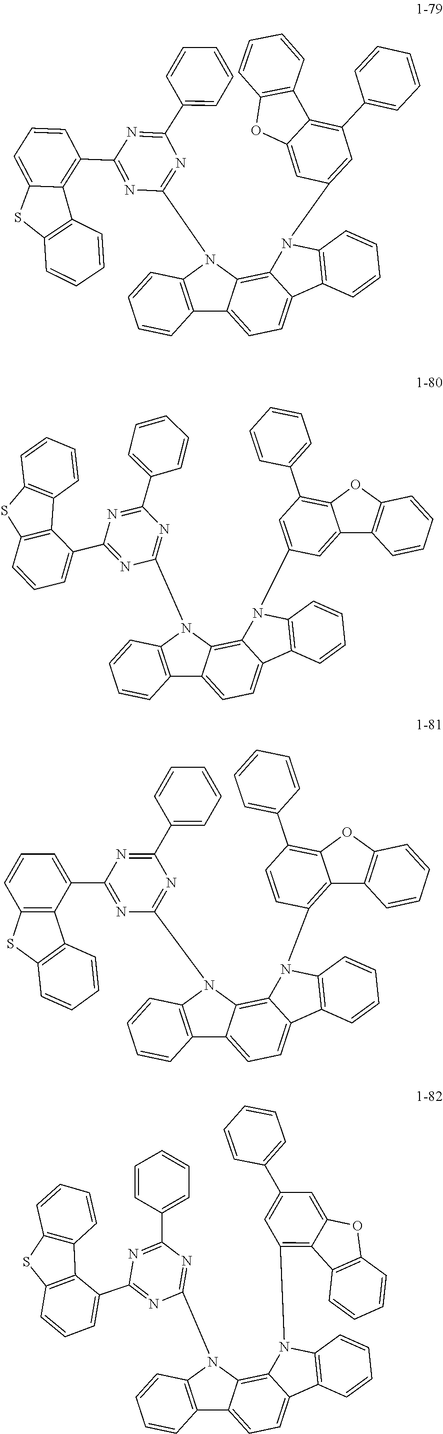

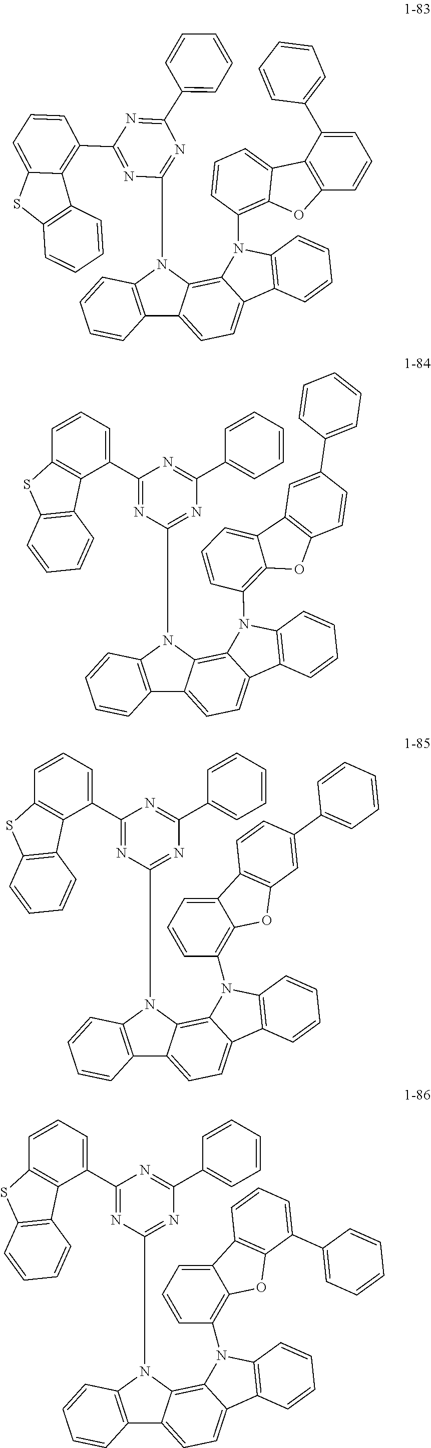

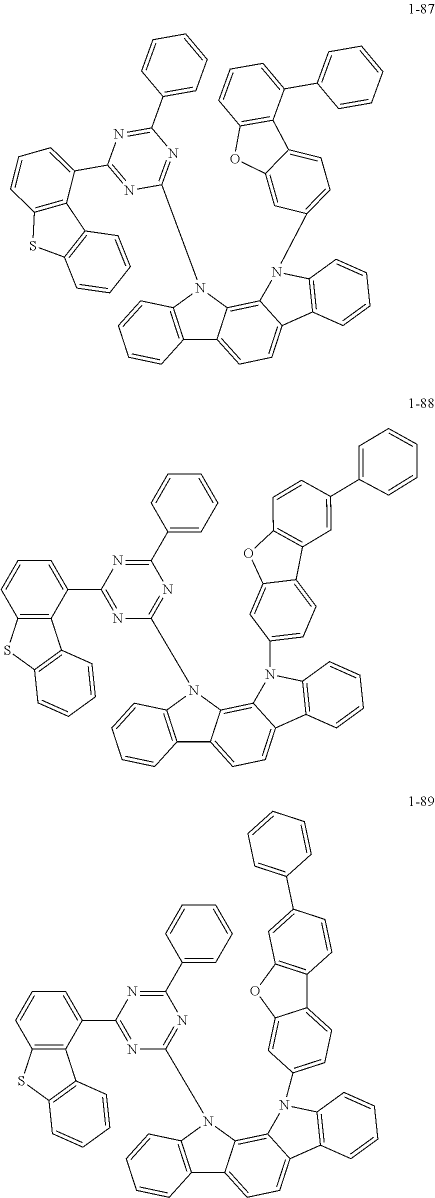 [SMILES: c1ccc(-c2nc(-c3cccc4sc5ccccc5c34)nc(-n3c4ccccc4c4ccc5c6ccccc6n(-c6ccc7c(c6)oc6cccc(-c8ccccc8)c67)c5c43)n2)cc1 ; c1ccc(-c2ccc3oc4cc(-n5c6ccccc6c6ccc7c8ccccc8n(-c8nc(-c9ccccc9)nc(-c9cccc%10sc%11ccccc%11c9%10)n8)c7c65)ccc4c3c2)cc1 ; c1ccc(-c2ccc3c(c2)oc2cc(-n4c5ccccc5c5ccc6c7ccccc7n(-c7nc(-c8ccccc8)nc(-c8cccc9sc%10ccccc%10c89)n7)c6c54)ccc23)cc1]

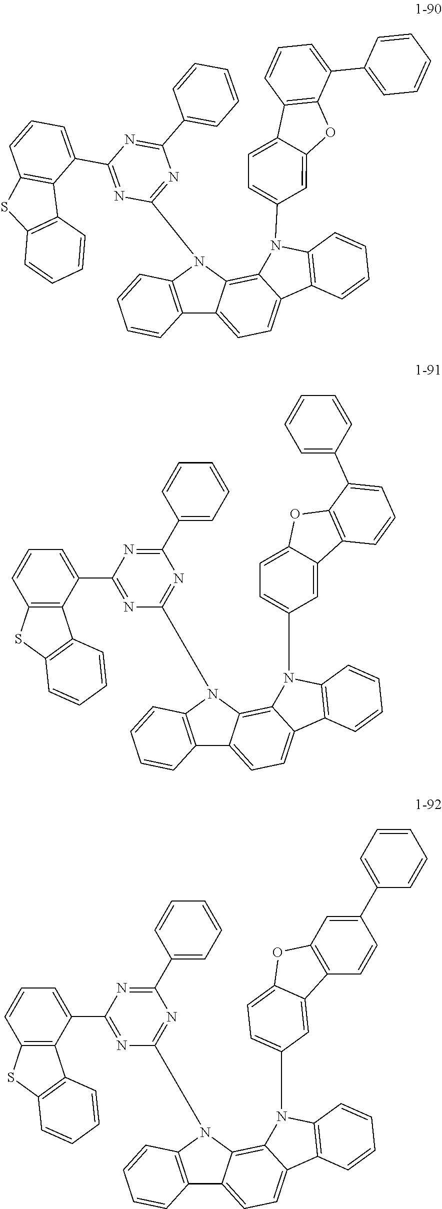 [SMILES: c1ccc(-c2nc(-c3cccc4sc5ccccc5c34)nc(-n3c4ccccc4c4ccc5c6ccccc6n(-c6ccc7c(c6)oc6c(-c8ccccc8)cccc67)c5c43)n2)cc1 ; c1ccc(-c2nc(-c3cccc4sc5ccccc5c34)nc(-n3c4ccccc4c4ccc5c6ccccc6n(-c6ccc7oc8c(-c9ccccc9)cccc8c7c6)c5c43)n2)cc1 ; c1ccc(-c2ccc3c(c2)oc2ccc(-n4c5ccccc5c5ccc6c7ccccc7n(-c7nc(-c8ccccc8)nc(-c8cccc9sc%10ccccc%10c89)n7)c6c54)cc23)cc1]

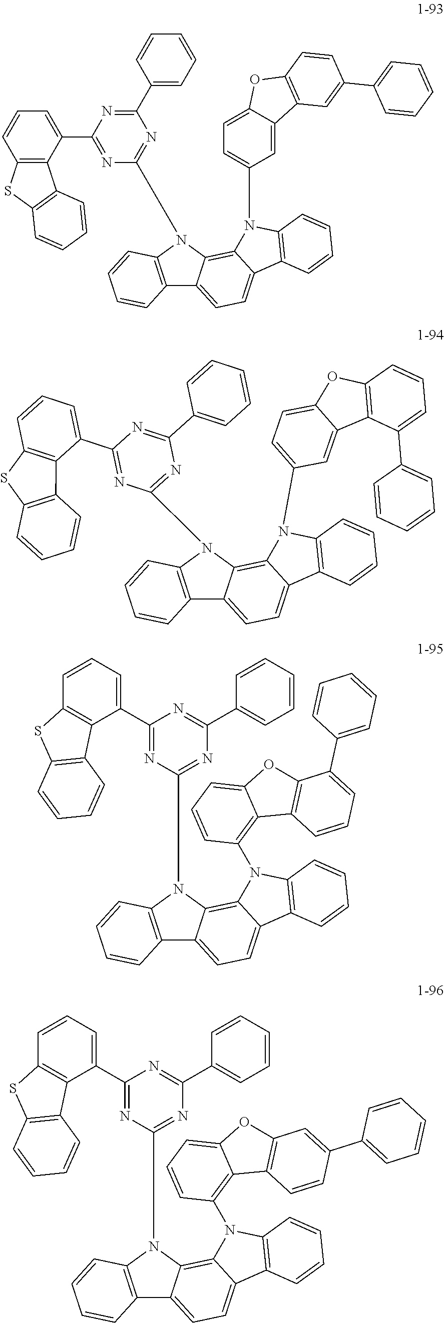

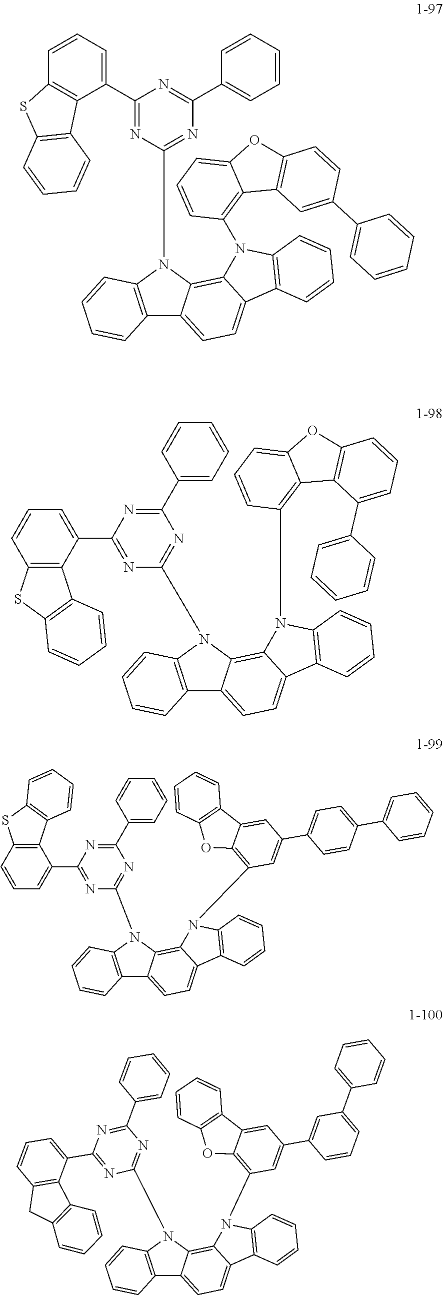 [SMILES: c1ccc(-c2ccc3oc4cccc(-n5c6ccccc6c6ccc7c8ccccc8n(-c8nc(-c9ccccc9)nc(-c9cccc%10sc%11ccccc%11c9%10)n8)c7c65)c4c3c2)cc1 ; c1ccc(-c2nc(-c3cccc4sc5ccccc5c34)nc(-n3c4ccccc4c4ccc5c6ccccc6n(-c6cccc7oc8cccc(-c9ccccc9)c8c67)c5c43)n2)cc1 ; c1ccc(-c2ccc(-c3cc(-n4c5ccccc5c5ccc6c7ccccc7n(-c7nc(-c8ccccc8)nc(-c8cccc9sc%10ccccc%10c89)n7)c6c54)c4oc5ccccc5c4c3)cc2)cc1 ; c1ccc(-c2cccc(-c3cc(-n4c5ccccc5c5ccc6c7ccccc7n(-c7nc(-c8ccccc8)nc(-c8cccc9c8-c8ccccc8C9)n7)c6c54)c4oc5ccccc5c4c3)c2)cc1]

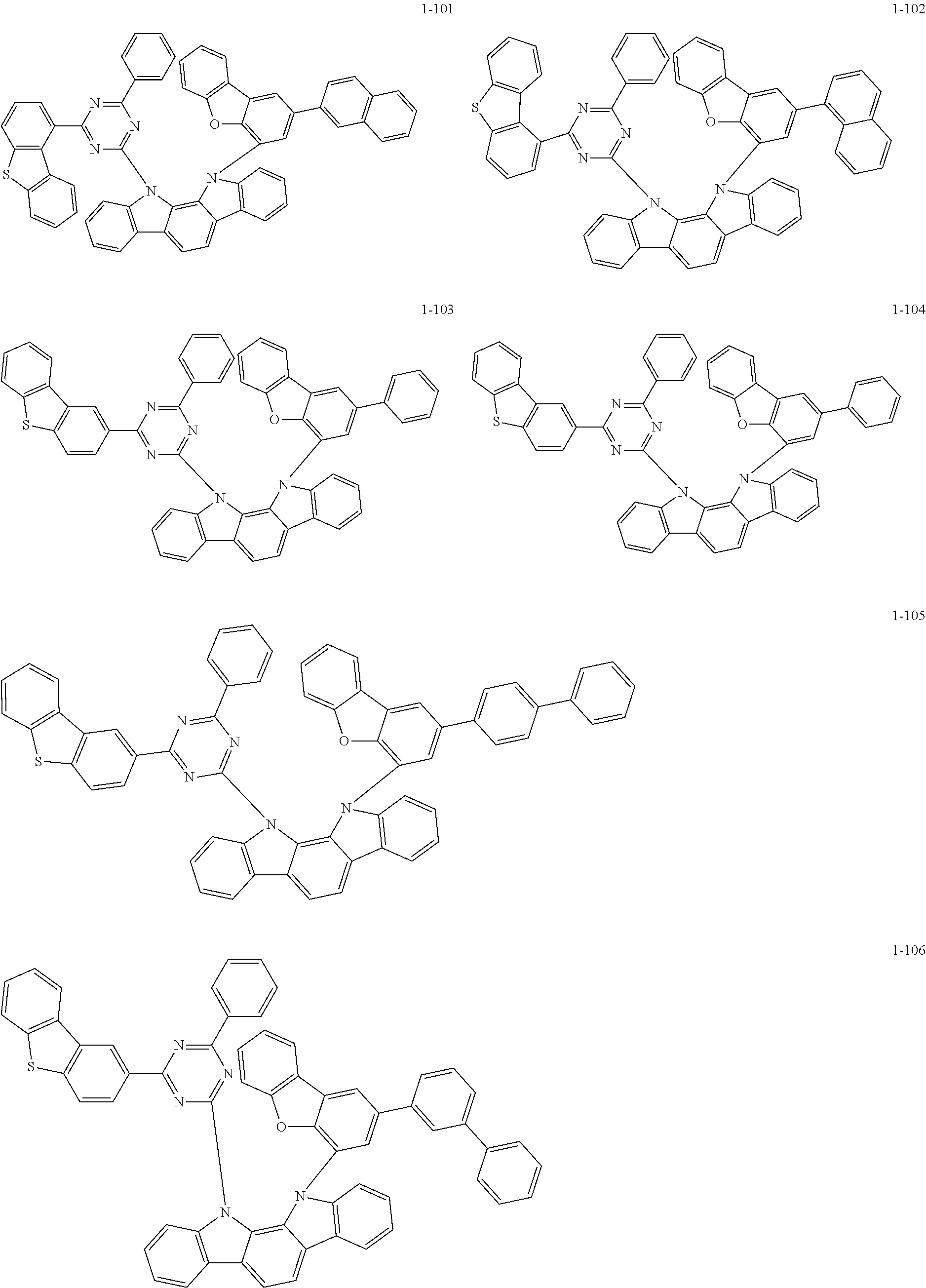

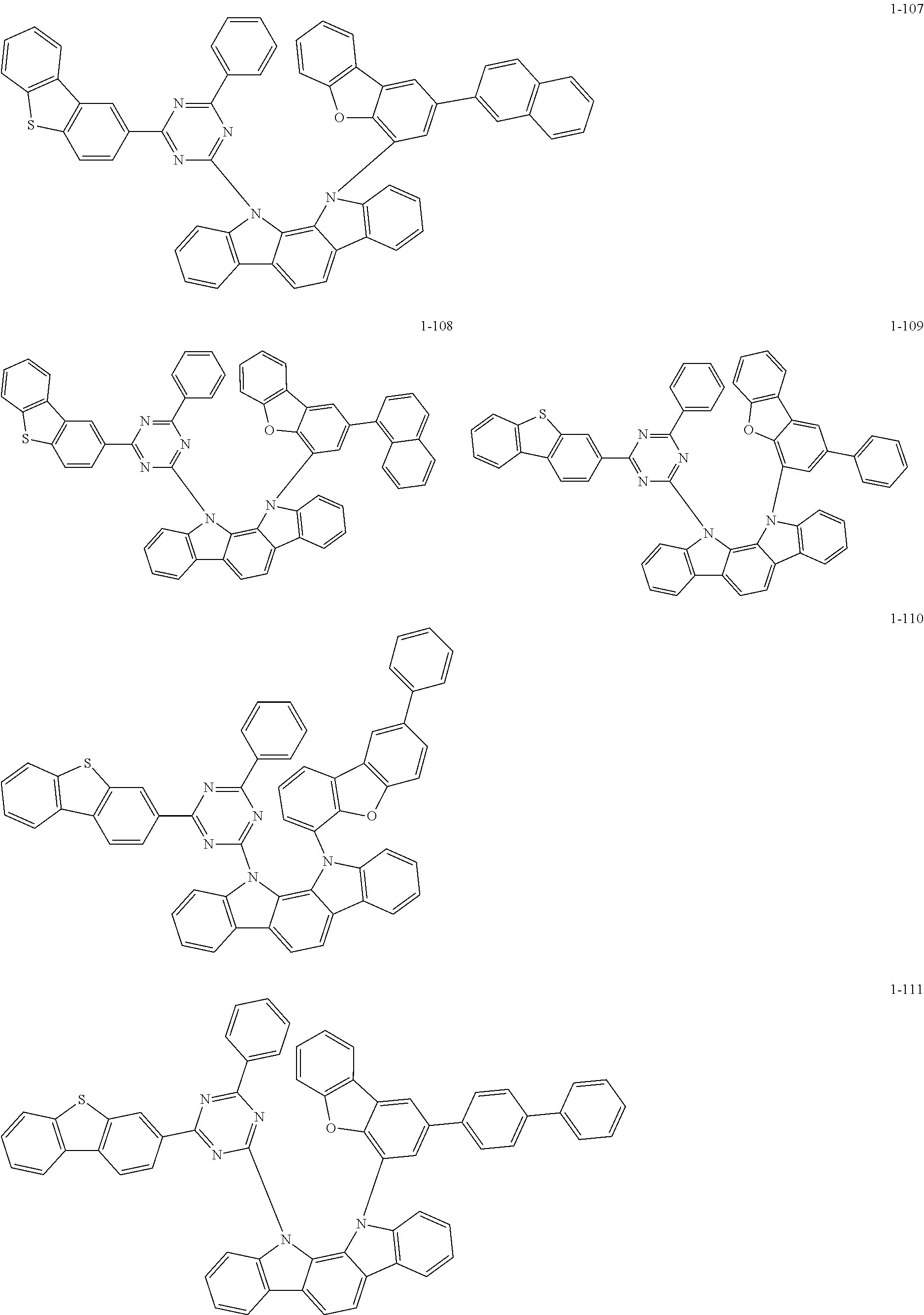

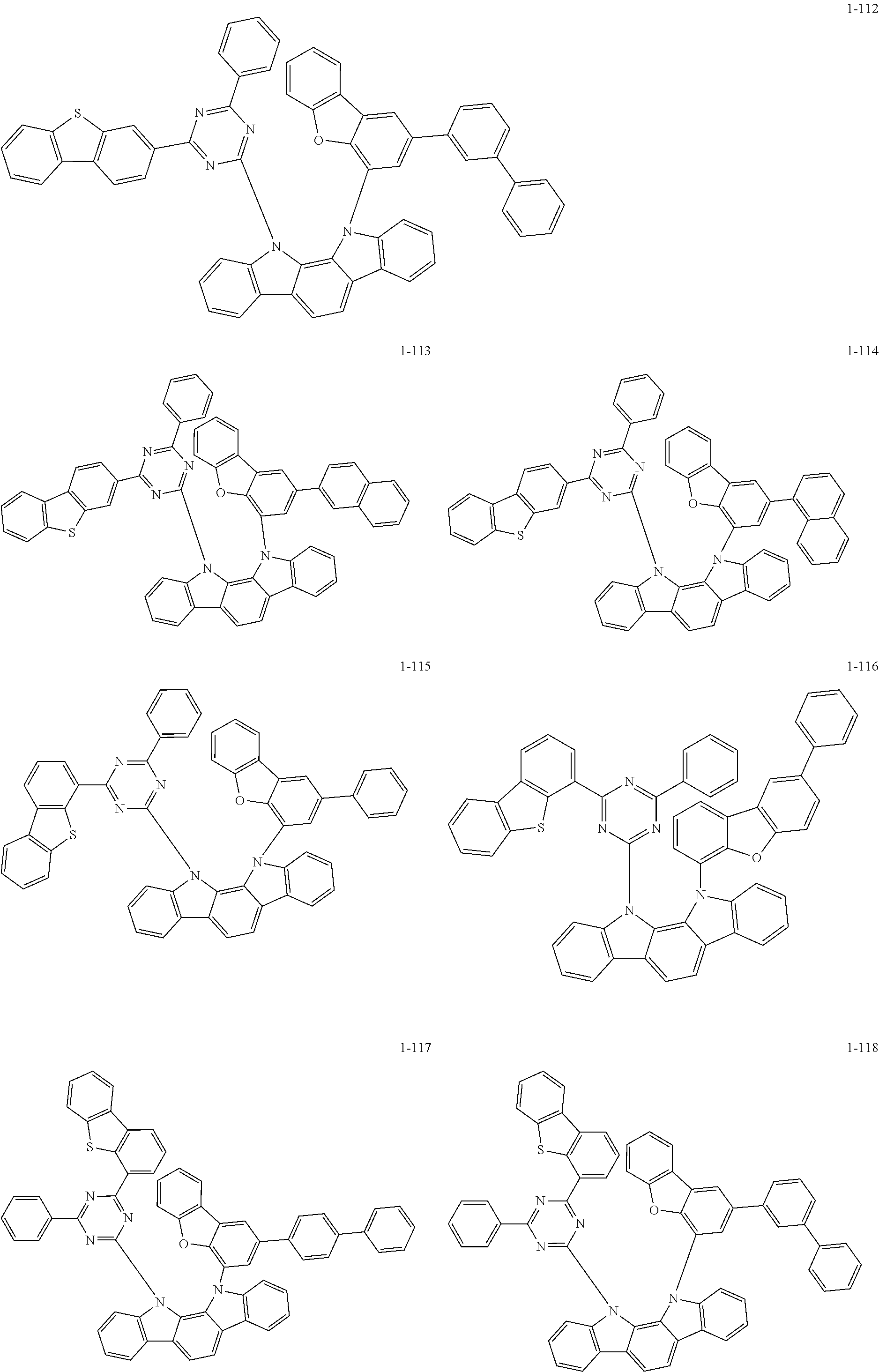

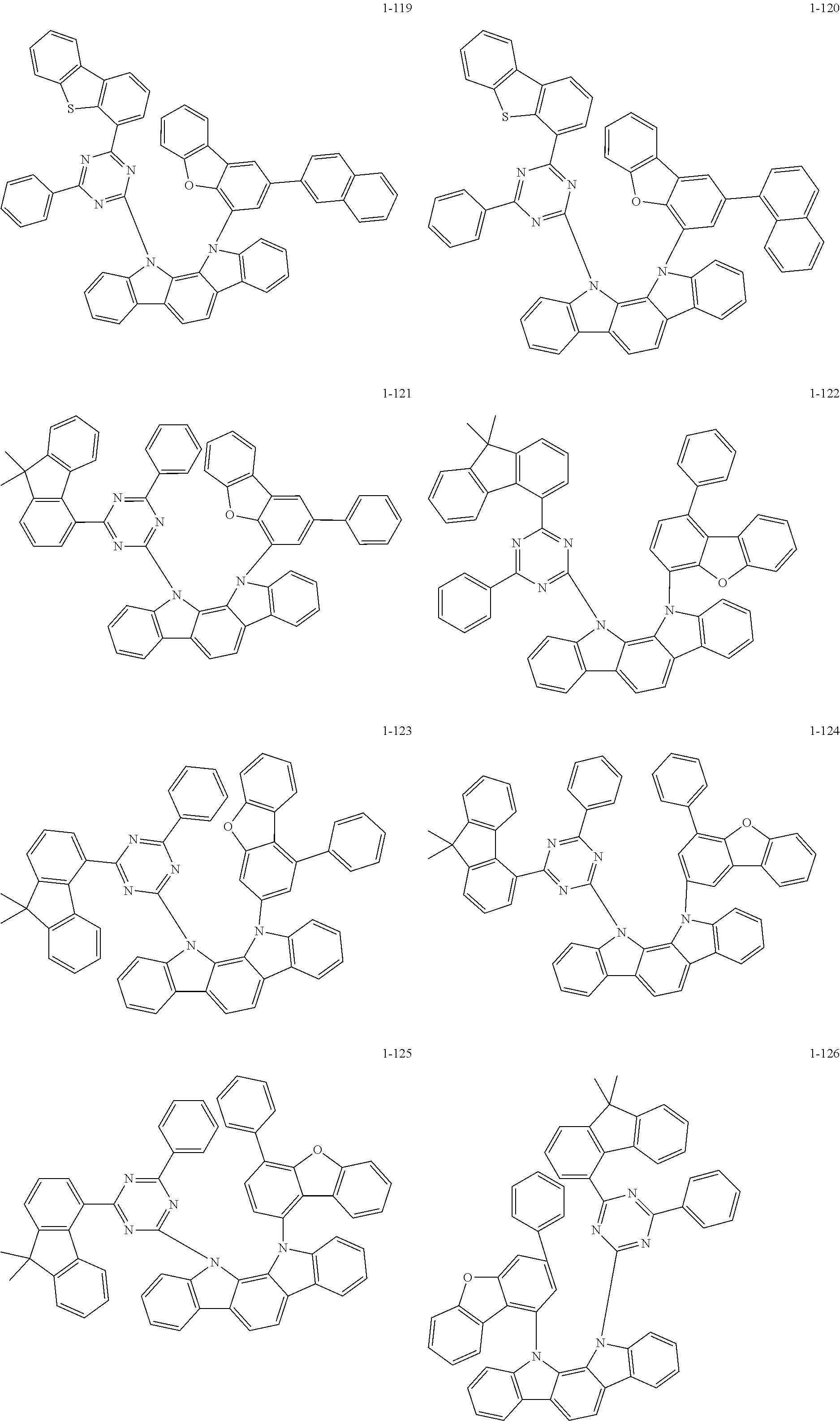

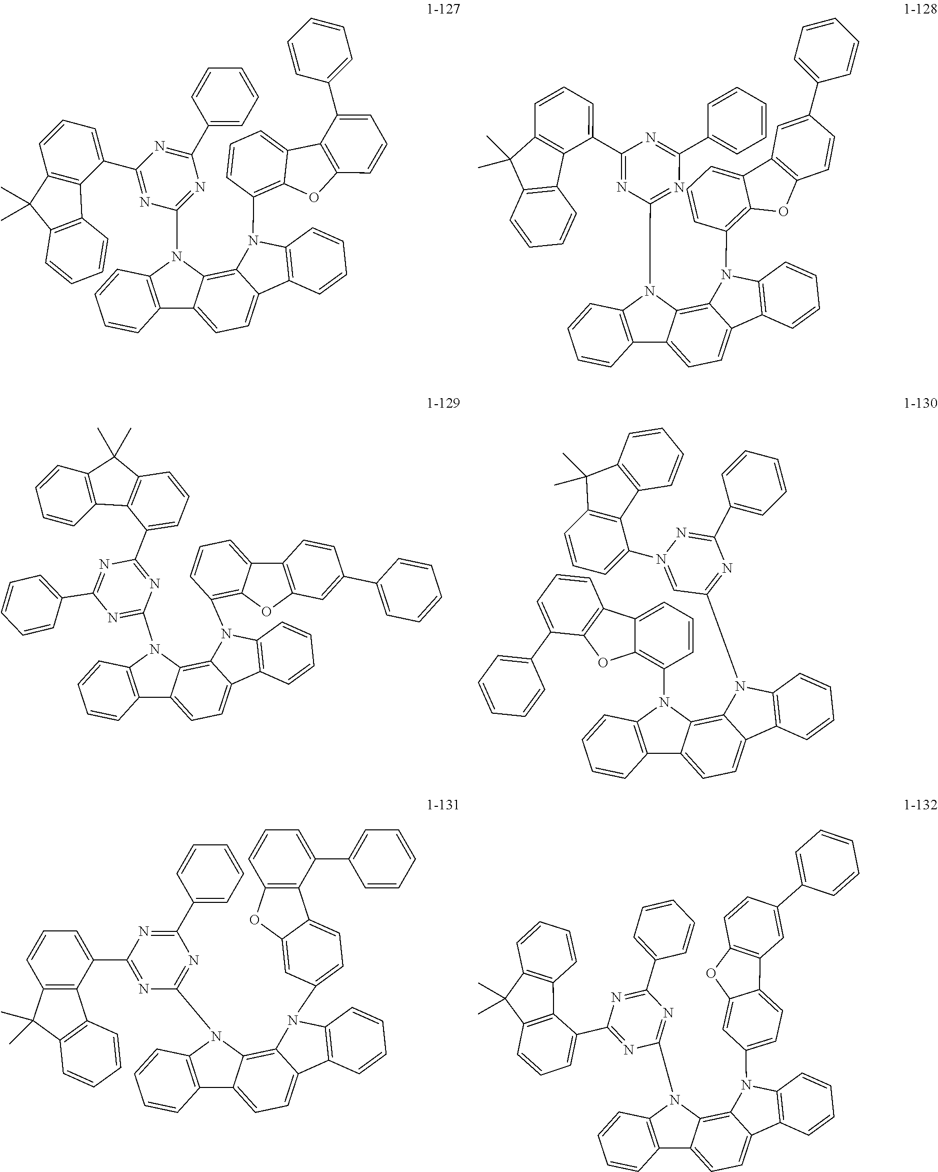

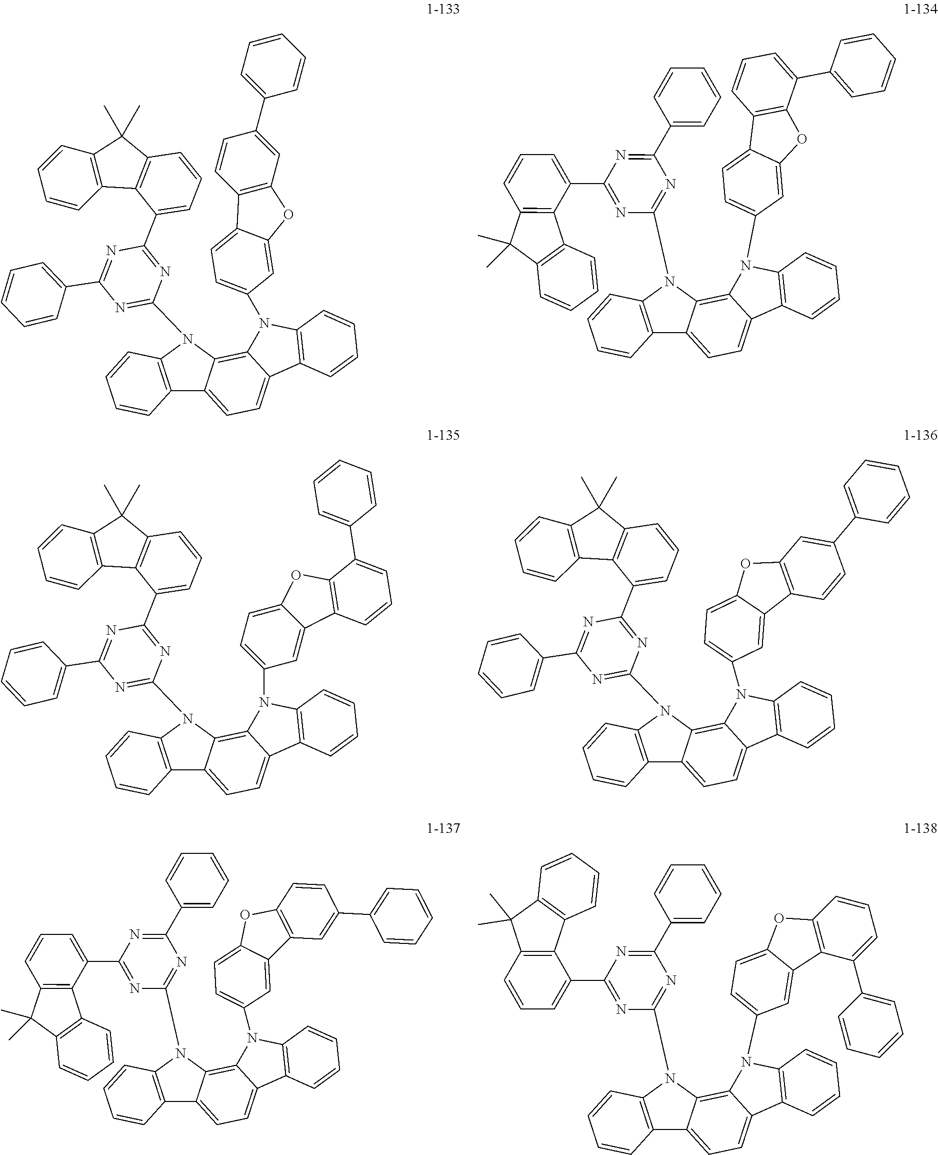

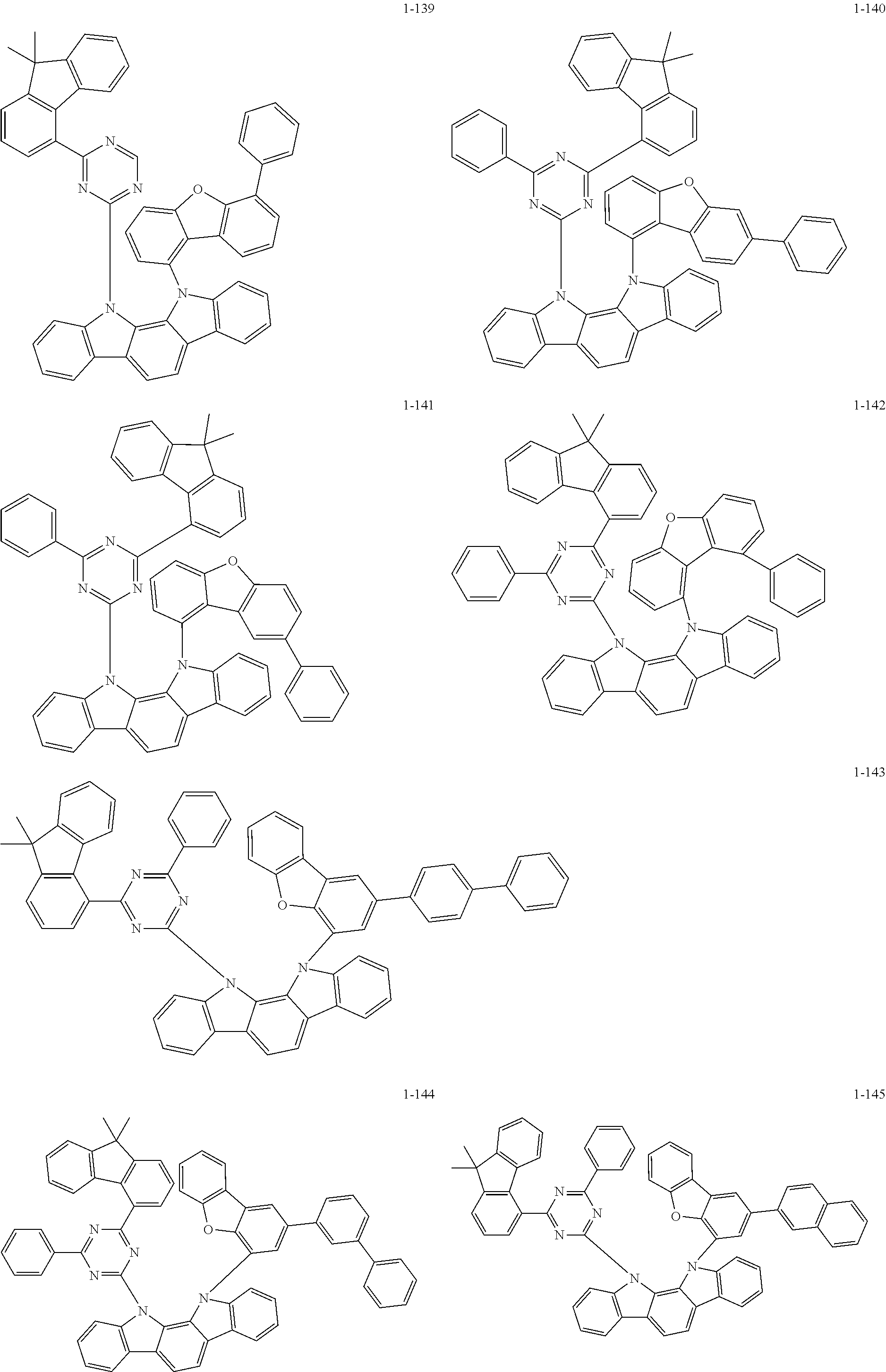

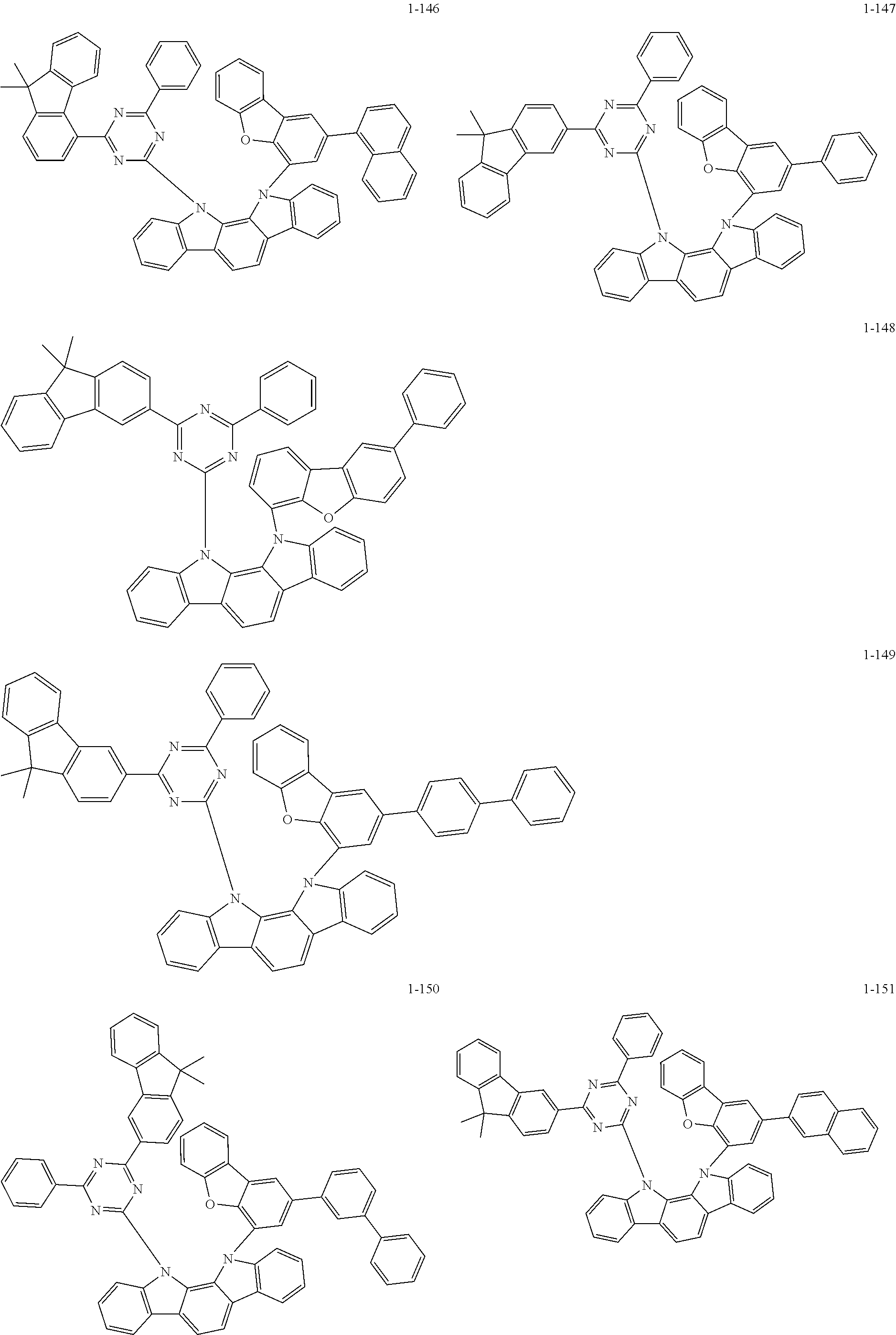

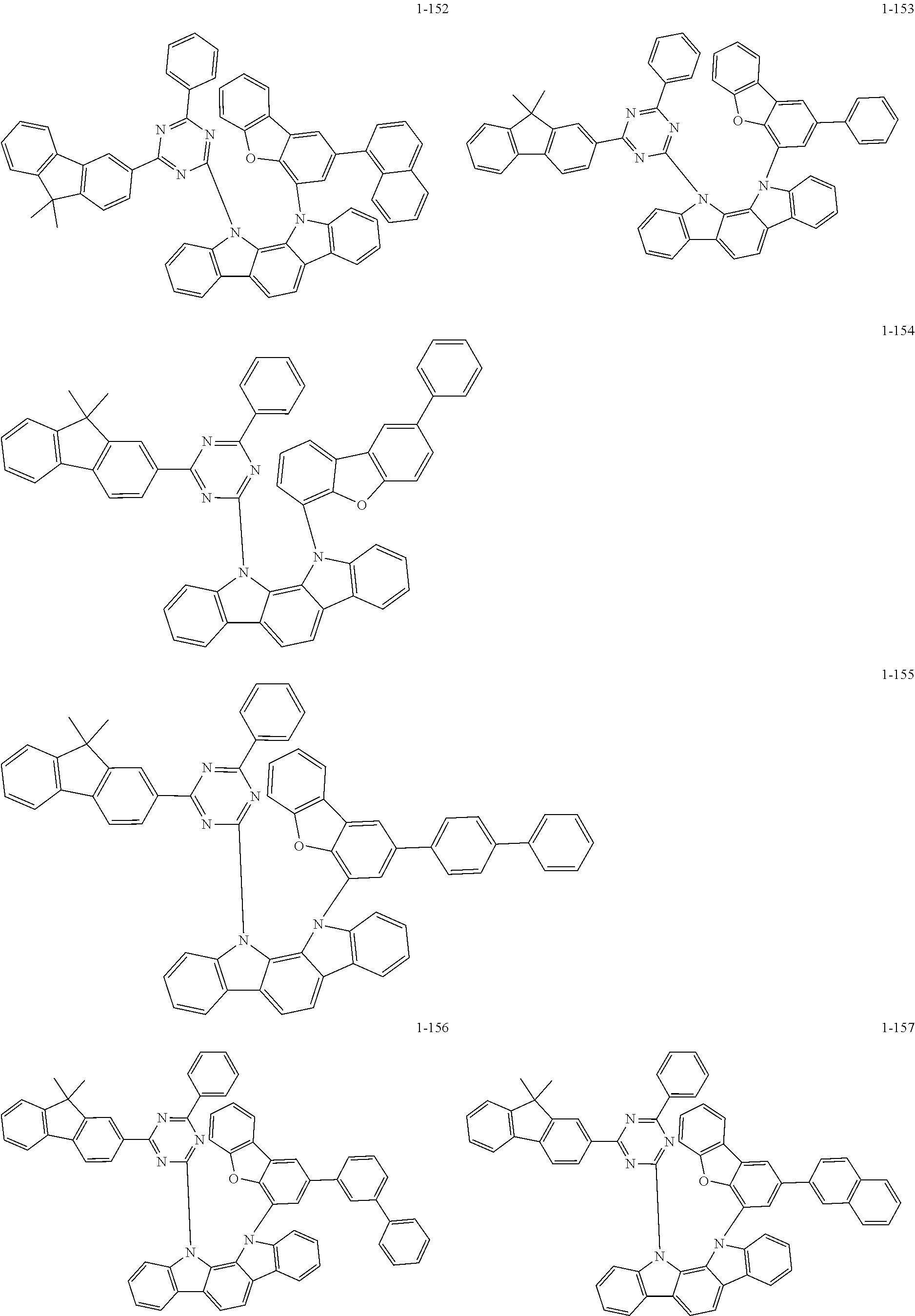

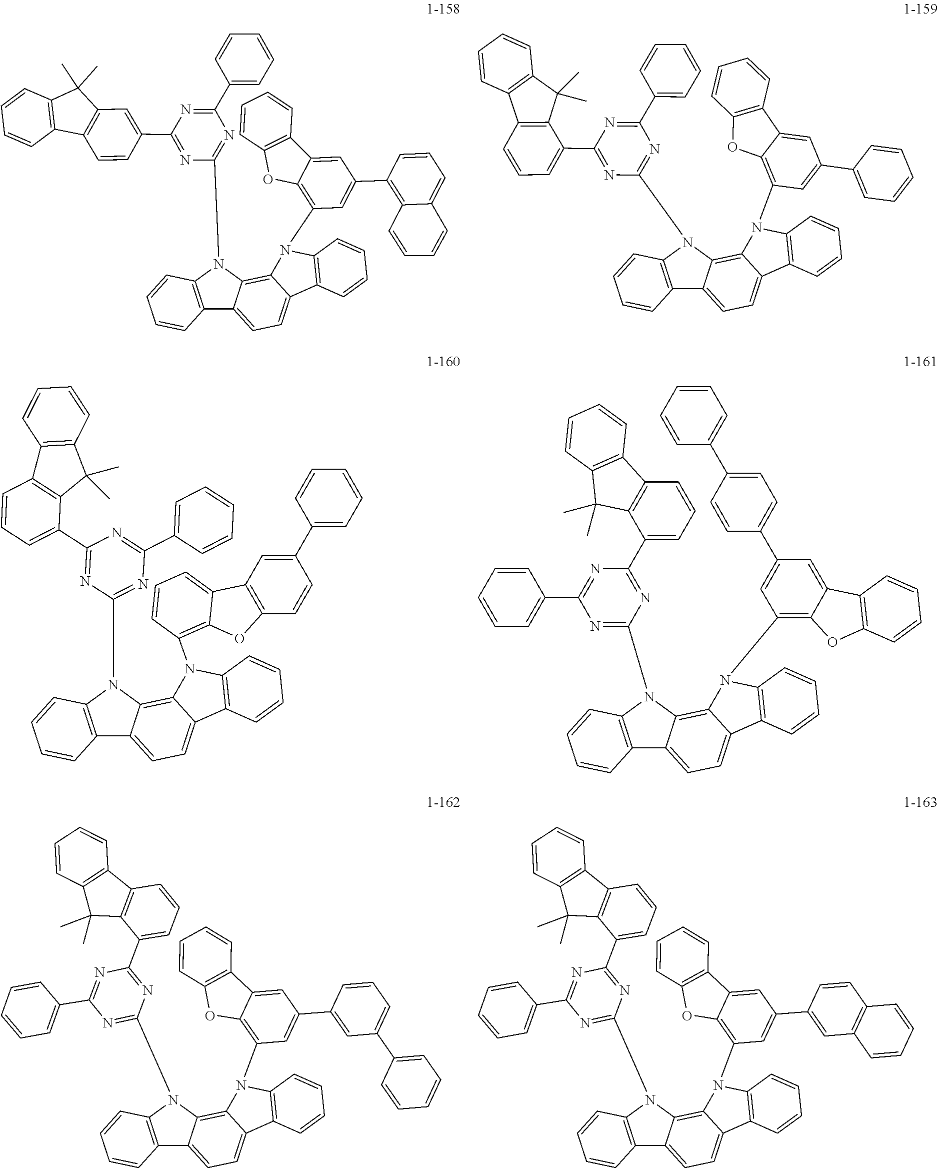

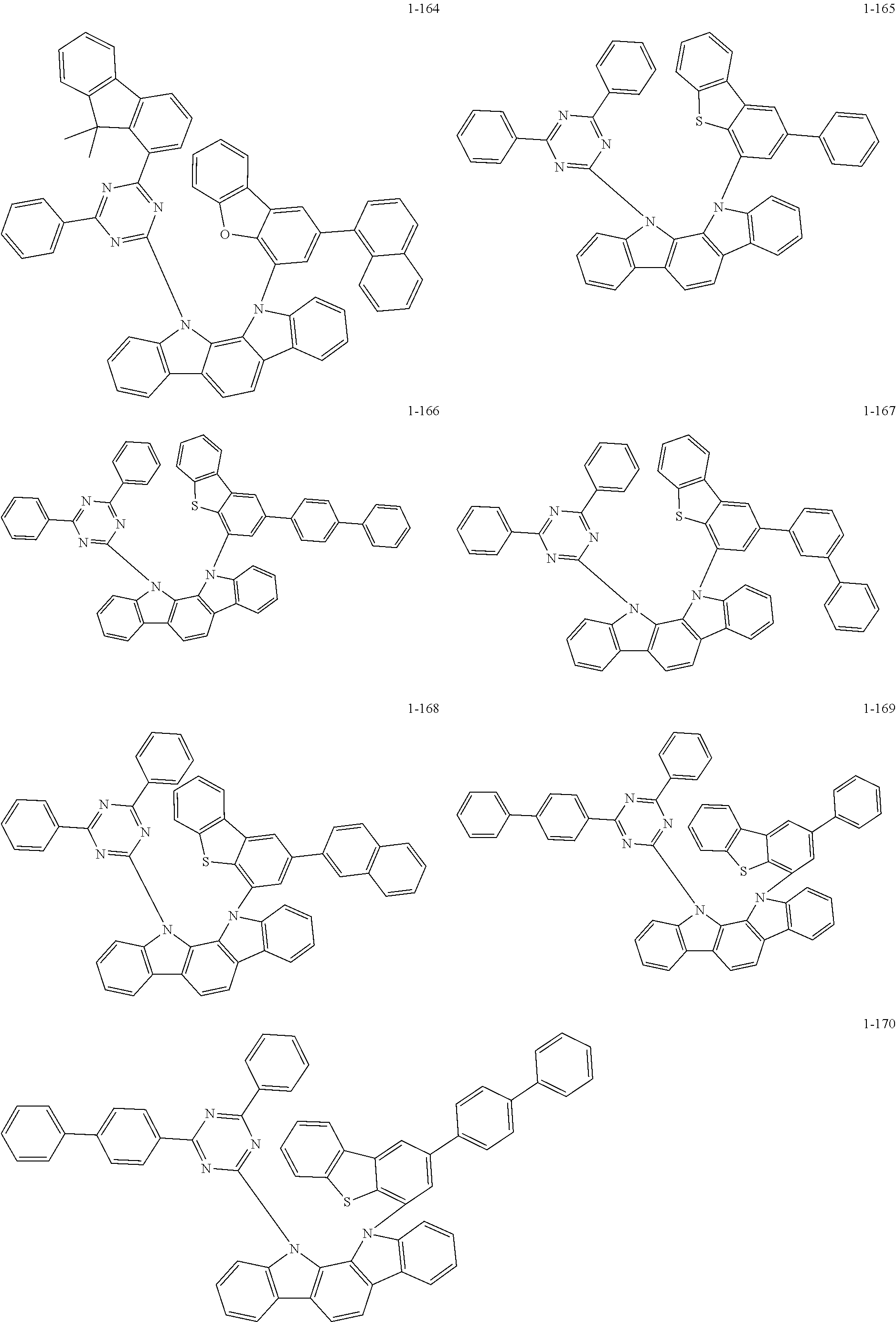

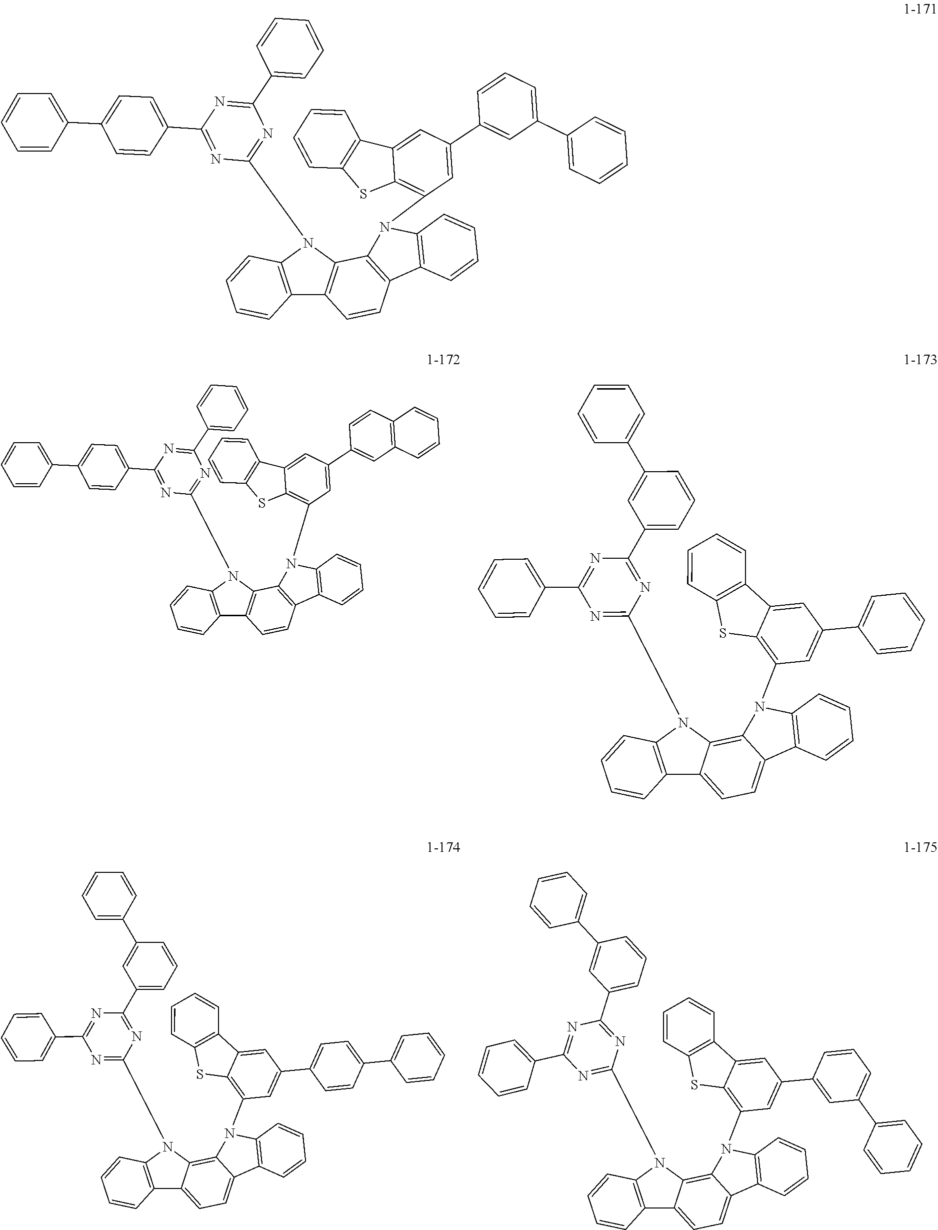

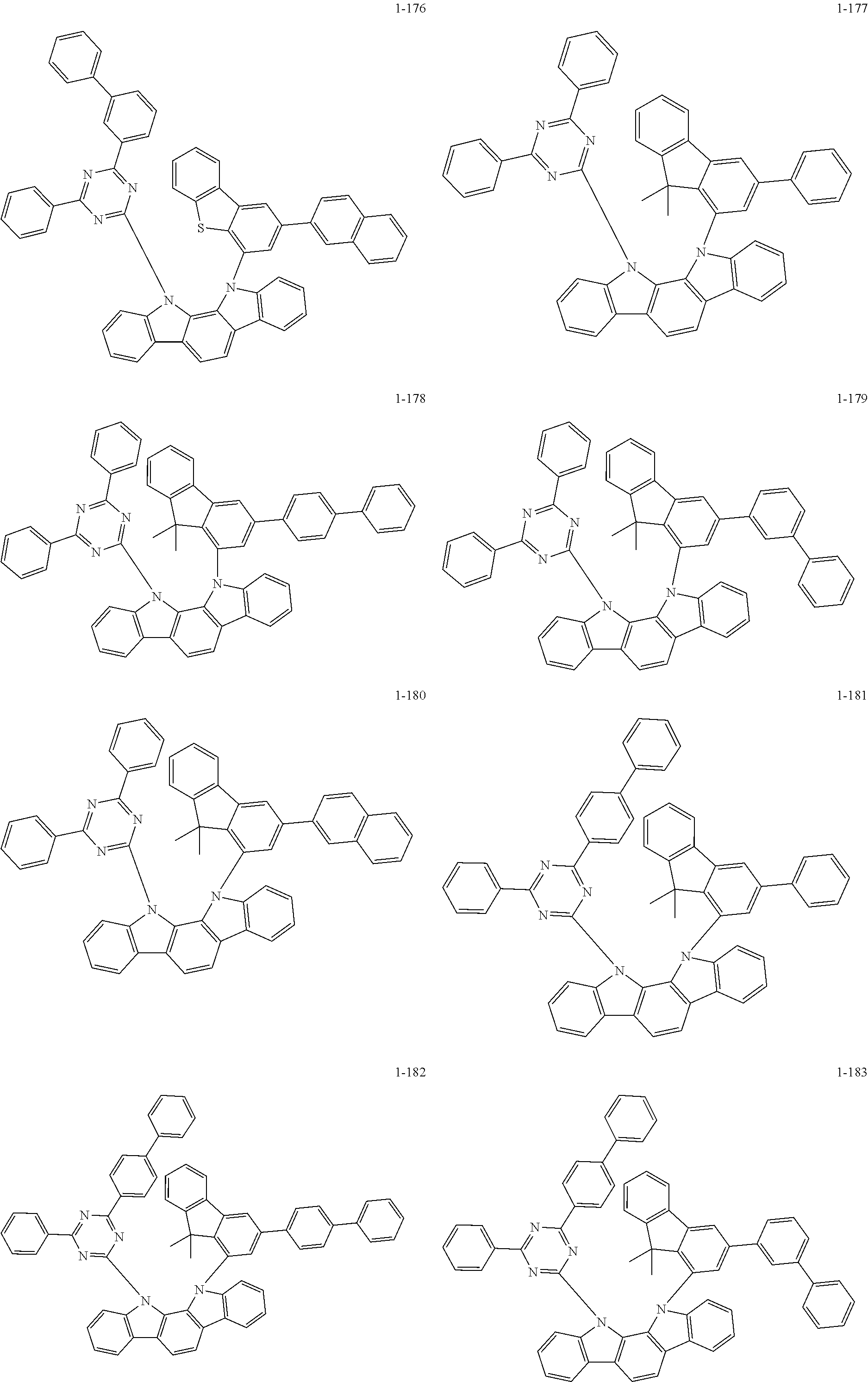

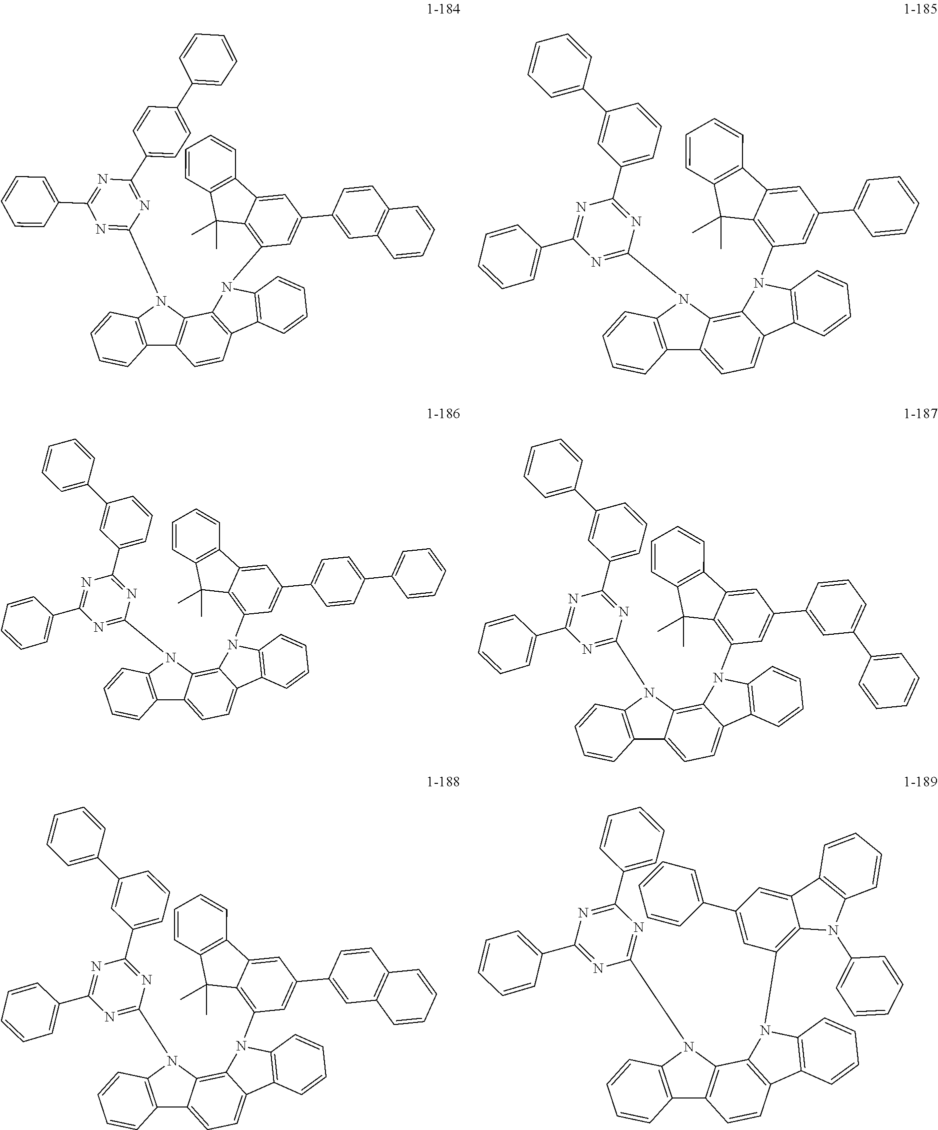

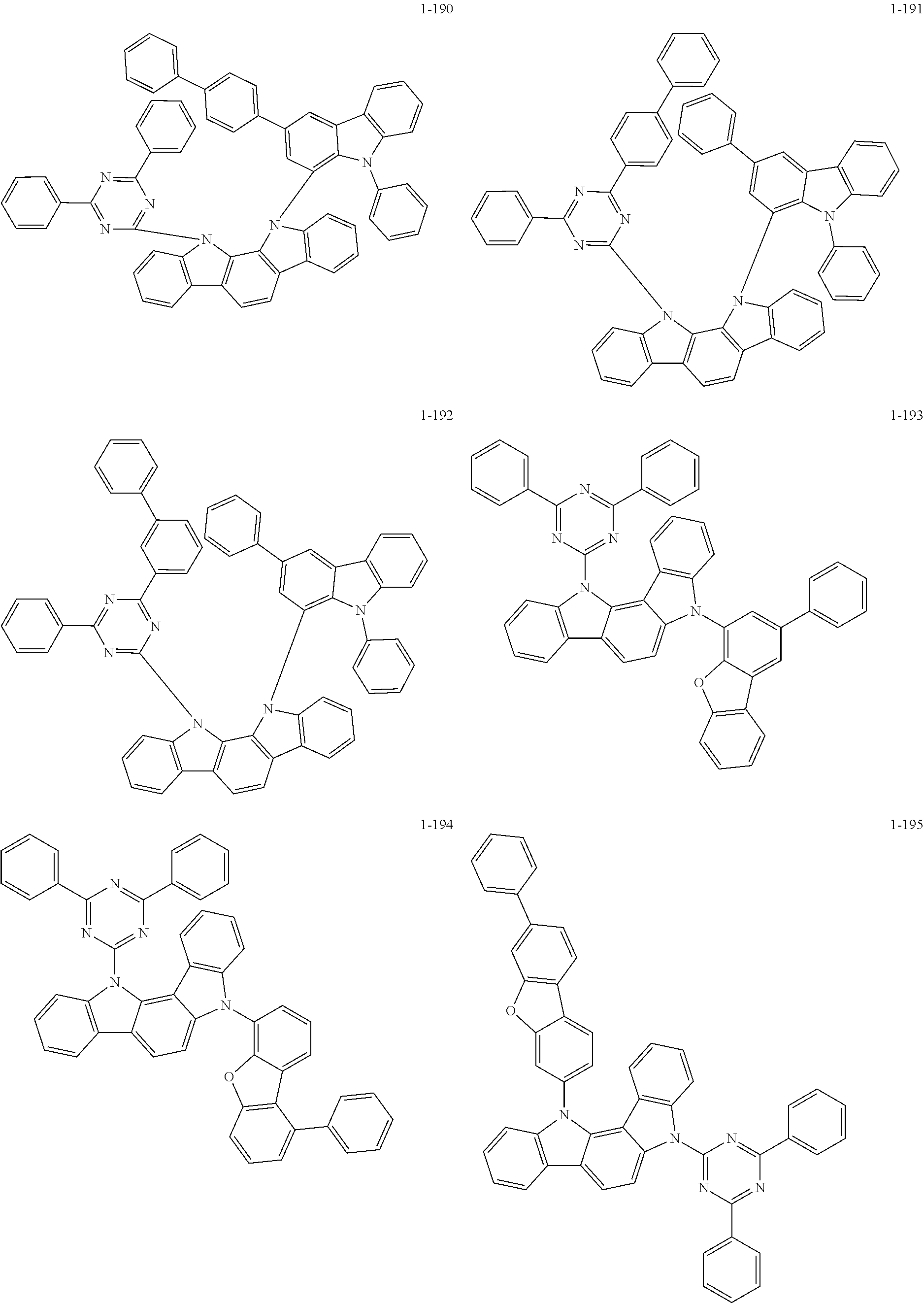

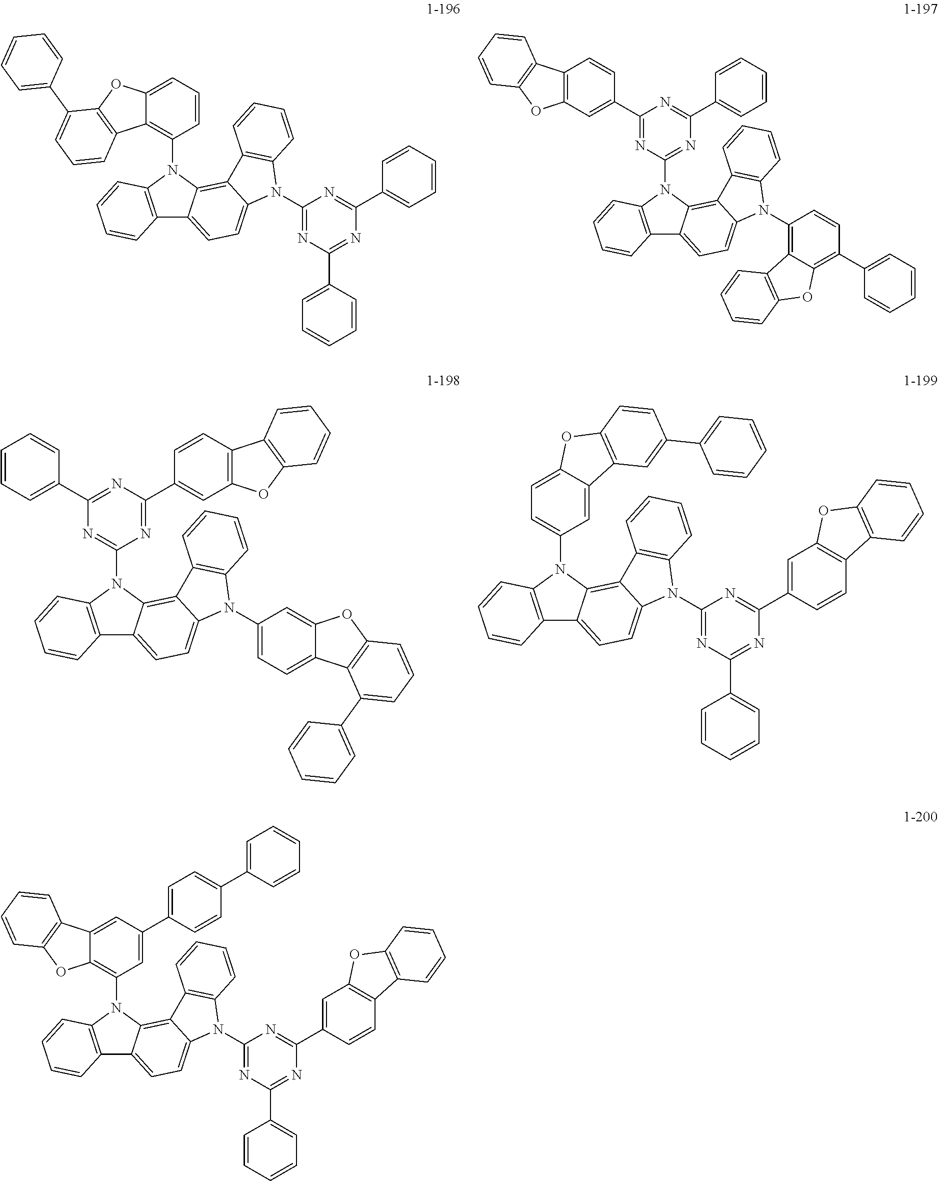

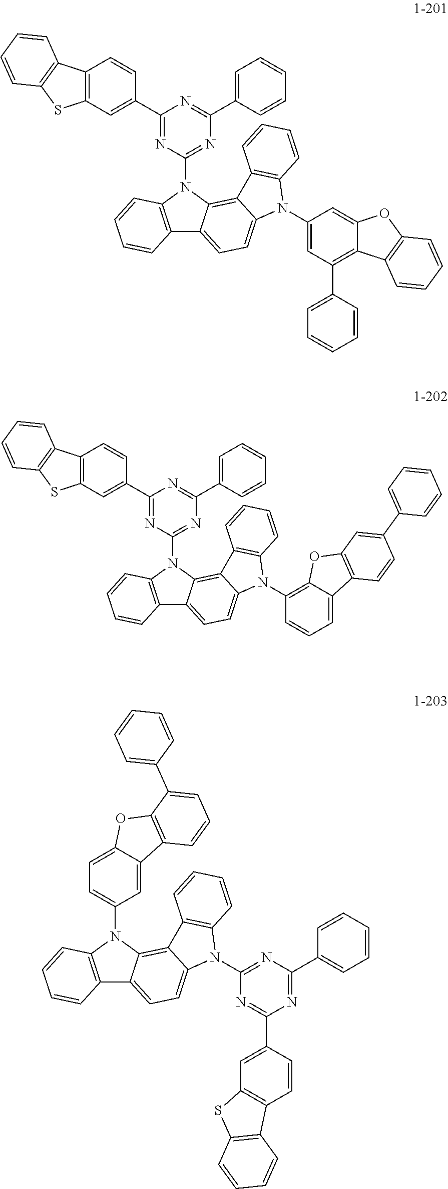 [SMILES: c1ccc(-c2nc(-c3ccc4c(c3)sc3ccccc34)nc(-n3c4ccccc4c4ccc5c(c6ccccc6n5-c5cc(-c6ccccc6)c6c(c5)oc5ccccc56)c43)n2)cc1 ; c1ccc(-c2ccc3c(c2)oc2c(-n4c5ccccc5c5c4ccc4c6ccccc6n(-c6nc(-c7ccccc7)nc(-c7ccc8c(c7)sc7ccccc78)n6)c45)cccc23)cc1 ; c1ccc(-c2nc(-c3ccc4c(c3)sc3ccccc34)nc(-n3c4ccccc4c4c3ccc3c5ccccc5n(-c5ccc6oc7c(-c8ccccc8)cccc7c6c5)c34)n2)cc1]

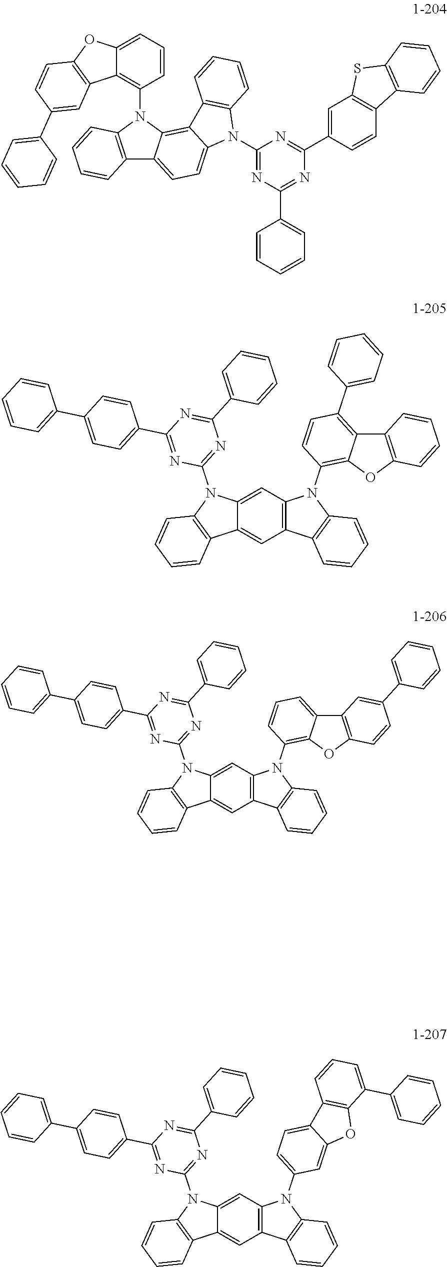 [SMILES: c1ccc(-c2ccc3oc4cccc(-n5c6ccccc6c6ccc7c(c8ccccc8n7-c7nc(-c8ccccc8)nc(-c8ccc9c(c8)sc8ccccc89)n7)c65)c4c3c2)cc1 ; c1ccc(-c2ccc(-c3nc(-c4ccccc4)nc(-n4c5ccccc5c5cc6c7ccccc7n(-c7ccc(-c8ccccc8)c8c7oc7ccccc78)c6cc54)n3)cc2)cc1 ; c1ccc(-c2ccc(-c3nc(-c4ccccc4)nc(-n4c5ccccc5c5cc6c7ccccc7n(-c7cccc8c7oc7ccc(-c9ccccc9)cc78)c6cc54)n3)cc2)cc1 ; c1ccc(-c2ccc(-c3nc(-c4ccccc4)nc(-n4c5ccccc5c5cc6c7ccccc7n(-c7ccc8c(c7)oc7c(-c9ccccc9)cccc78)c6cc54)n3)cc2)cc1]

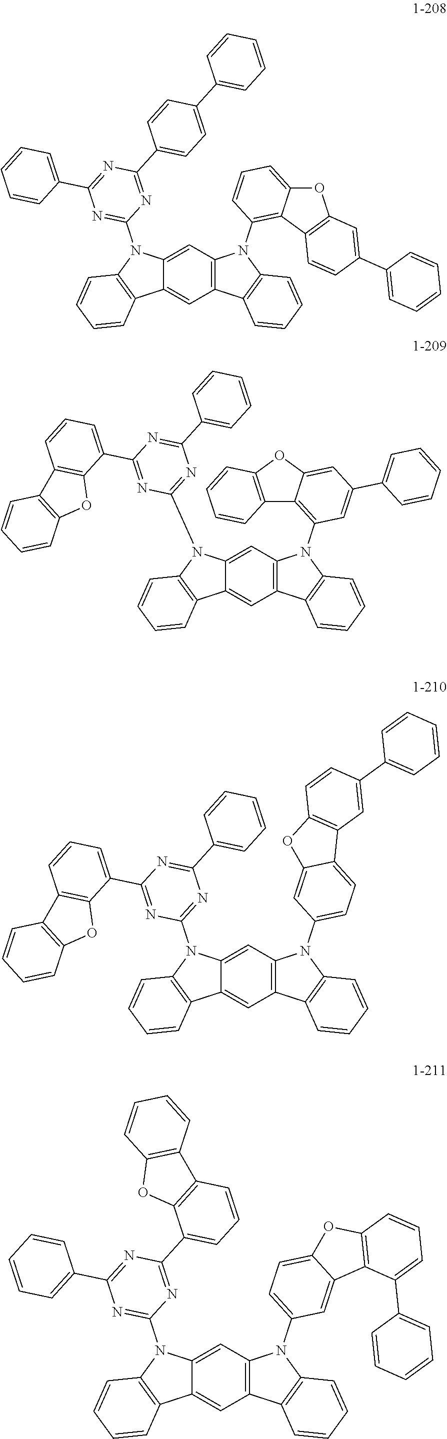 [SMILES: c1ccc(-c2ccc(-c3nc(-c4ccccc4)nc(-n4c5ccccc5c5cc6c7ccccc7n(-c7cccc8oc9cc(-c%10ccccc%10)ccc9c78)c6cc54)n3)cc2)cc1 ; c1ccc(-c2cc(-n3c4ccccc4c4cc5c6ccccc6n(-c6nc(-c7ccccc7)nc(-c7cccc8c7oc7ccccc78)n6)c5cc43)c3c(c2)oc2ccccc23)cc1 ; c1ccc(-c2ccc3oc4cc(-n5c6ccccc6c6cc7c8ccccc8n(-c8nc(-c9ccccc9)nc(-c9cccc%10c9oc9ccccc9%10)n8)c7cc65)ccc4c3c2)cc1 ; c1ccc(-c2nc(-c3cccc4c3oc3ccccc34)nc(-n3c4ccccc4c4cc5c6ccccc6n(-c6ccc7oc8cccc(-c9ccccc9)c8c7c6)c5cc43)n2)cc1]

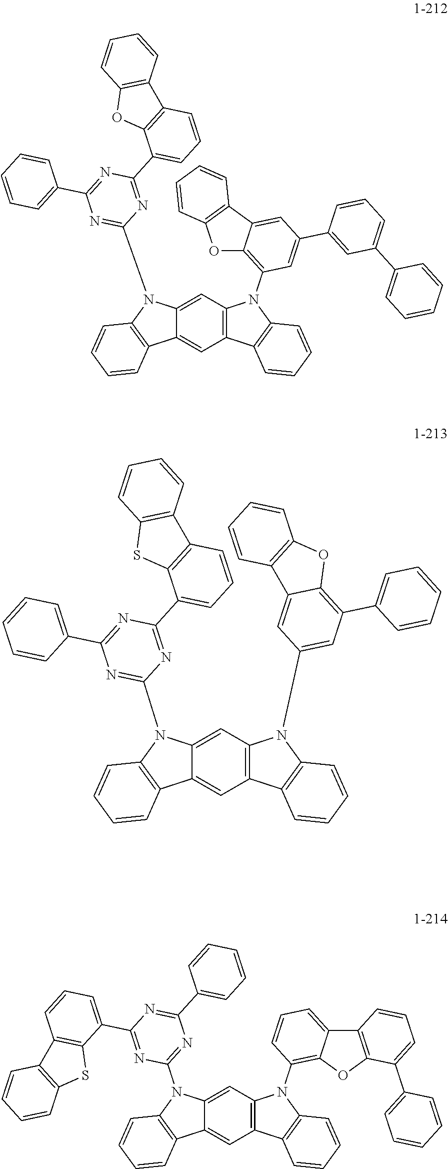 [SMILES: c1ccc(-c2cccc(-c3cc(-n4c5ccccc5c5cc6c7ccccc7n(-c7nc(-c8ccccc8)nc(-c8cccc9c8oc8ccccc89)n7)c6cc54)c4oc5ccccc5c4c3)c2)cc1 ; c1ccc(-c2nc(-c3cccc4c3sc3ccccc34)nc(-n3c4ccccc4c4cc5c6ccccc6n(-c6cc(-c7ccccc7)c7oc8ccccc8c7c6)c5cc43)n2)cc1 ; c1ccc(-c2nc(-c3cccc4c3sc3ccccc34)nc(-n3c4ccccc4c4cc5c6ccccc6n(-c6cccc7c6oc6c(-c8ccccc8)cccc67)c5cc43)n2)cc1]

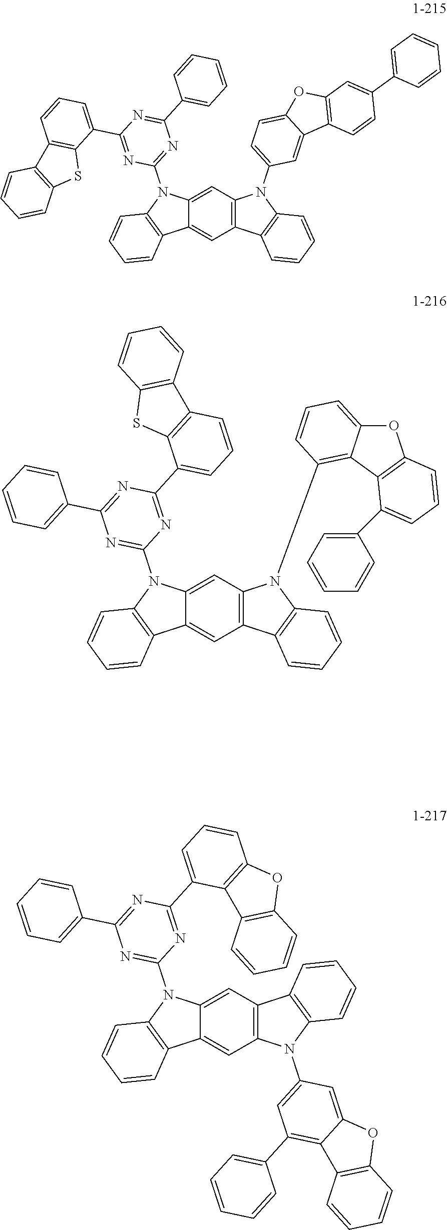 [SMILES: c1ccc(-c2ccc3c(c2)oc2ccc(-n4c5ccccc5c5cc6c7ccccc7n(-c7nc(-c8ccccc8)nc(-c8cccc9c8sc8ccccc89)n7)c6cc54)cc23)cc1 ; c1ccc(-c2nc(-c3cccc4c3sc3ccccc34)nc(-n3c4ccccc4c4cc5c6ccccc6n(-c6cccc7oc8cccc(-c9ccccc9)c8c67)c5cc43)n2)cc1 ; c1ccc(-c2nc(-c3cccc4oc5ccccc5c34)nc(-n3c4ccccc4c4cc5c(cc43)c3ccccc3n5-c3cc(-c4ccccc4)c4c(c3)oc3ccccc34)n2)cc1]

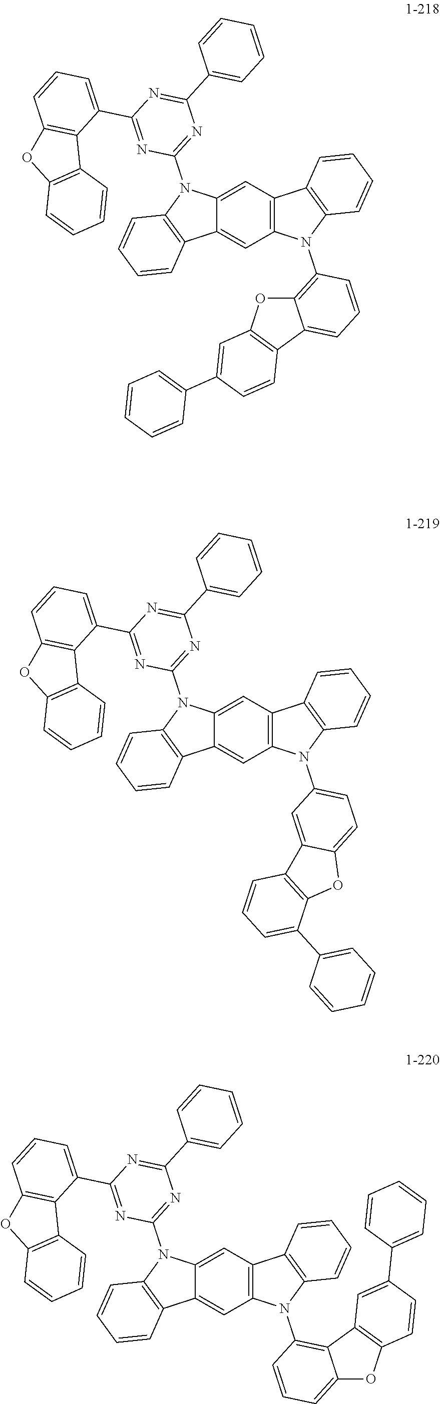 [SMILES: c1ccc(-c2ccc3c(c2)oc2c(-n4c5ccccc5c5cc6c(cc54)c4ccccc4n6-c4nc(-c5ccccc5)nc(-c5cccc6oc7ccccc7c56)n4)cccc23)cc1 ; c1ccc(-c2nc(-c3cccc4oc5ccccc5c34)nc(-n3c4ccccc4c4cc5c(cc43)c3ccccc3n5-c3ccc4oc5c(-c6ccccc6)cccc5c4c3)n2)cc1 ; c1ccc(-c2ccc3oc4cccc(-n5c6ccccc6c6cc7c(cc65)c5ccccc5n7-c5nc(-c6ccccc6)nc(-c6cccc7oc8ccccc8c67)n5)c4c3c2)cc1]

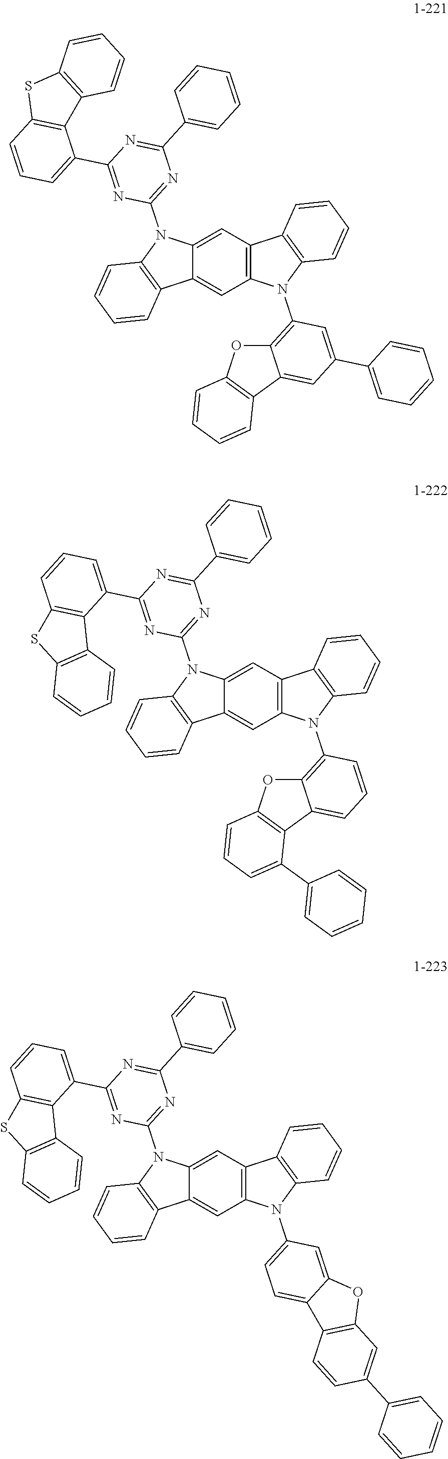 [SMILES: c1ccc(-c2cc(-n3c4ccccc4c4cc5c(cc43)c3ccccc3n5-c3nc(-c4ccccc4)nc(-c4cccc5sc6ccccc6c45)n3)c3oc4ccccc4c3c2)cc1 ; c1ccc(-c2nc(-c3cccc4sc5ccccc5c34)nc(-n3c4ccccc4c4cc5c(cc43)c3ccccc3n5-c3cccc4c3oc3cccc(-c5ccccc5)c34)n2)cc1 ; c1ccc(-c2ccc3c(c2)oc2cc(-n4c5ccccc5c5cc6c(cc54)c4ccccc4n6-c4nc(-c5ccccc5)nc(-c5cccc6sc7ccccc7c56)n4)ccc23)cc1]

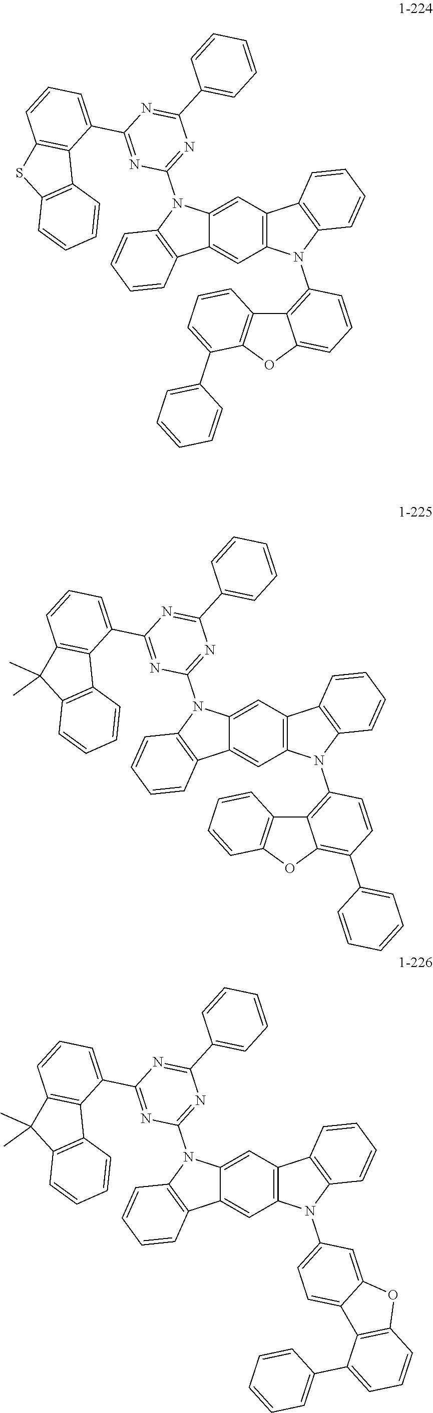 [SMILES: c1ccc(-c2nc(-c3cccc4sc5ccccc5c34)nc(-n3c4ccccc4c4cc5c(cc43)c3ccccc3n5-c3cccc4oc5c(-c6ccccc6)cccc5c34)n2)cc1 ; CC1(C)c2ccccc2-c2c(-c3nc(-c4ccccc4)nc(-n4c5ccccc5c5cc6c(cc54)c4ccccc4n6-c4ccc(-c5ccccc5)c5oc6ccccc6c45)n3)cccc21 ; CC1(C)c2ccccc2-c2c(-c3nc(-c4ccccc4)nc(-n4c5ccccc5c5cc6c(cc54)c4ccccc4n6-c4ccc5c(c4)oc4cccc(-c6ccccc6)c45)n3)cccc21]

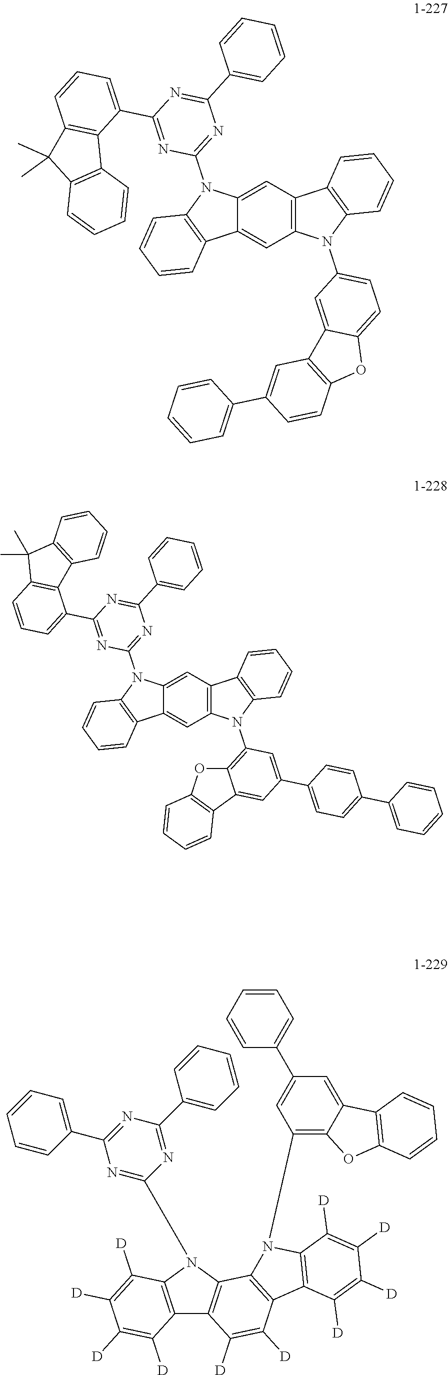 [SMILES: CC1(C)c2ccccc2-c2c(-c3nc(-c4ccccc4)nc(-n4c5ccccc5c5cc6c(cc54)c4ccccc4n6-c4ccc5oc6ccc(-c7ccccc7)cc6c5c4)n3)cccc21 ; CC1(C)c2ccccc2-c2c(-c3nc(-c4ccccc4)nc(-n4c5ccccc5c5cc6c(cc54)c4ccccc4n6-c4cc(-c5ccc(-c6ccccc6)cc5)cc5c4oc4ccccc45)n3)cccc21 ; [2H]c1c([2H])c([2H])c2c(c1[2H])c1c([2H])c([2H])c3c4c([2H])c([2H])c([2H])c([2H])c4n(-c4cc(-c5ccccc5)cc5c4oc4ccccc45)c3c1n2-c1nc(-c2ccccc2)nc(-c2ccccc2)n1]

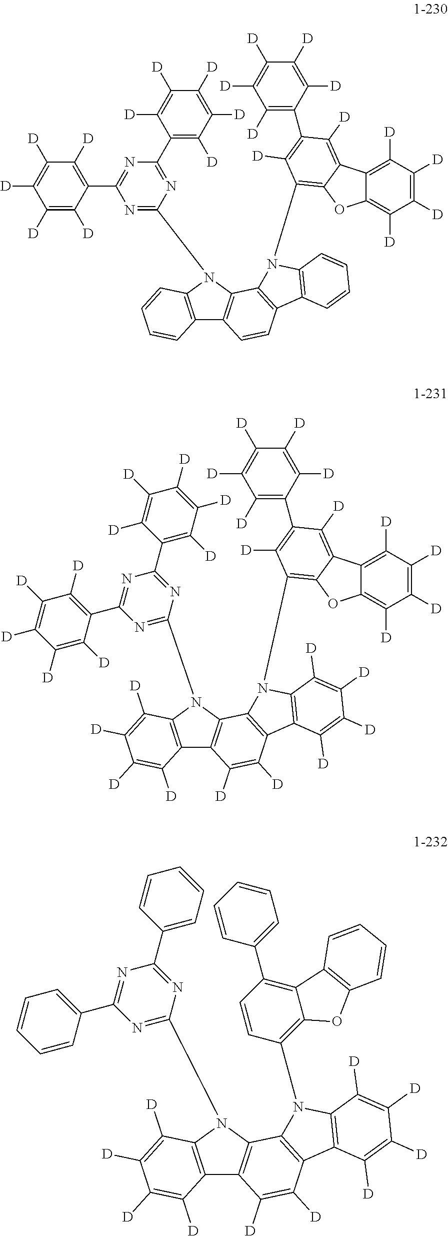

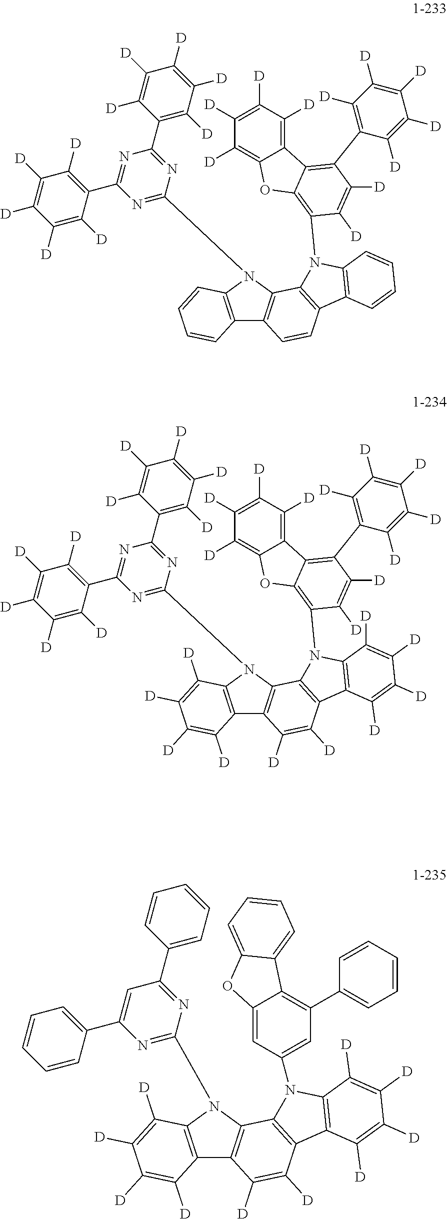

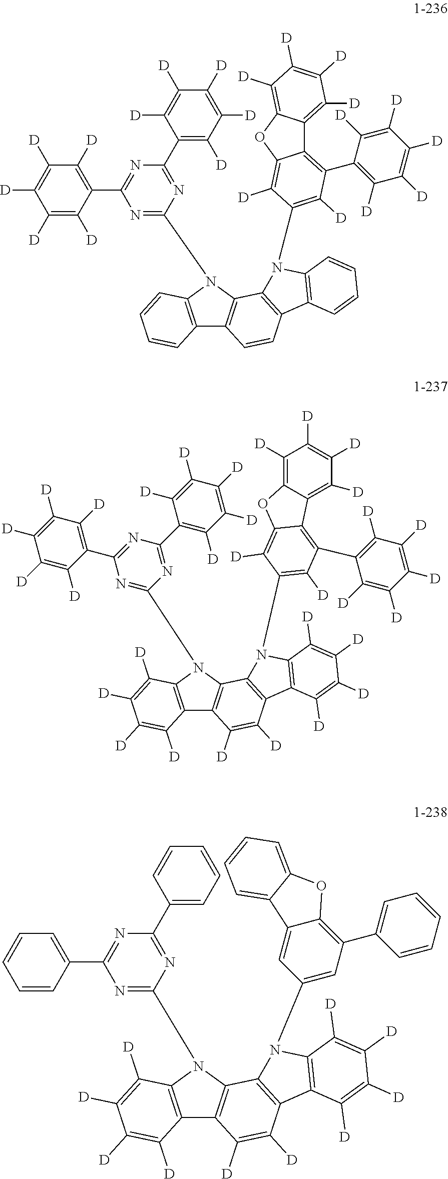

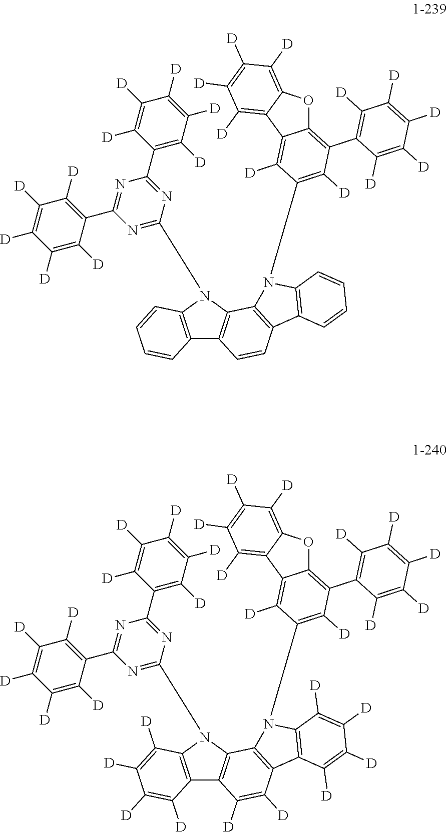 [SMILES: [2H]c1c([2H])c([2H])c(-c2nc(-c3c([2H])c([2H])c([2H])c([2H])c3[2H])nc(-n3c4ccccc4c4ccc5c6ccccc6n(-c6c([2H])c(-c7c([2H])c([2H])c([2H])c([2H])c7[2H])c7oc8c([2H])c([2H])c([2H])c([2H])c8c7c6[2H])c5c43)n2)c([2H])c1[2H] ; [2H]c1c([2H])c([2H])c(-c2nc(-c3c([2H])c([2H])c([2H])c([2H])c3[2H])nc(-n3c4c([2H])c([2H])c([2H])c([2H])c4c4c([2H])c([2H])c5c6c([2H])c([2H])c([2H])c([2H])c6n(-c6c([2H])c(-c7c([2H])c([2H])c([2H])c([2H])c7[2H])c7oc8c([2H])c([2H])c([2H])c([2H])c8c7c6[2H])c5c43)n2)c([2H])c1[2H]]

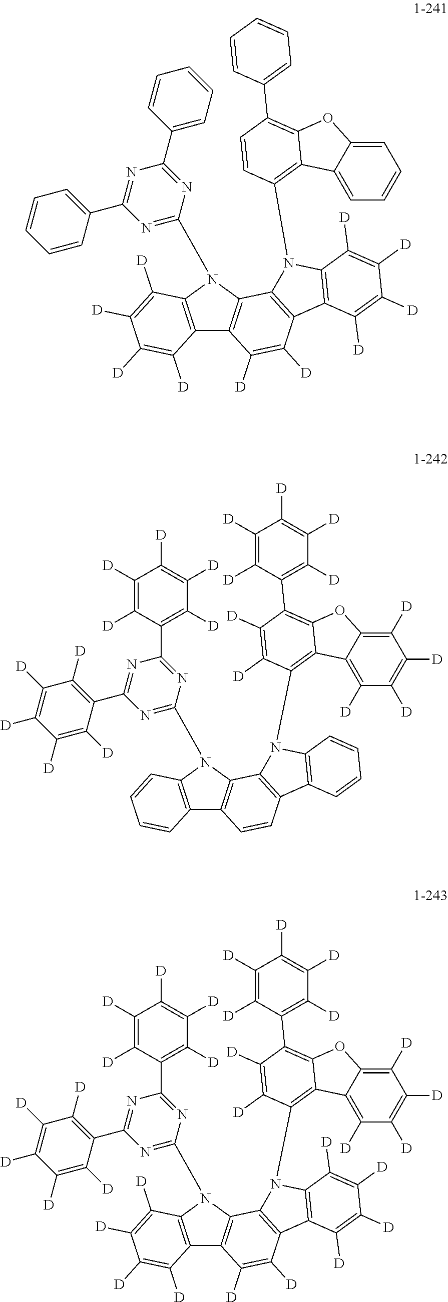

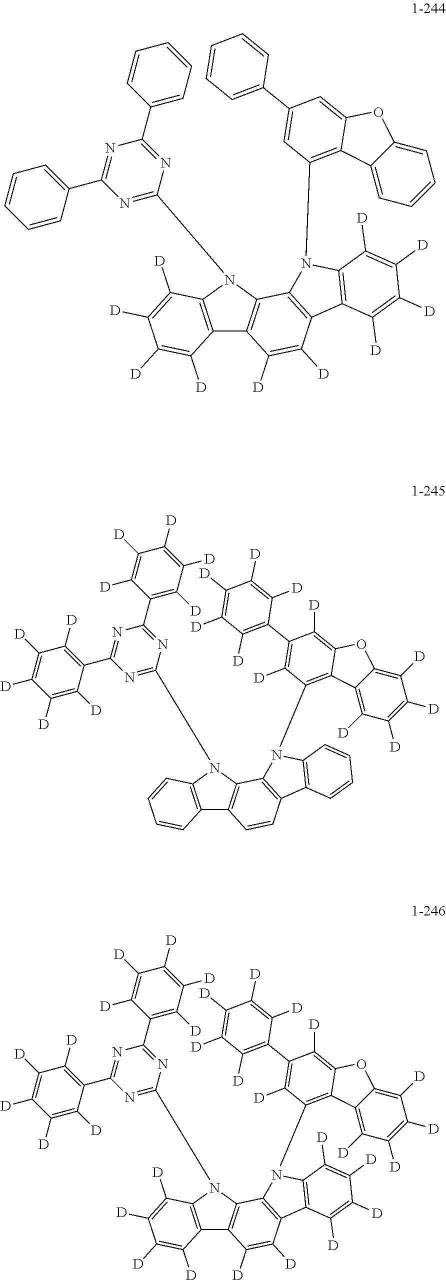

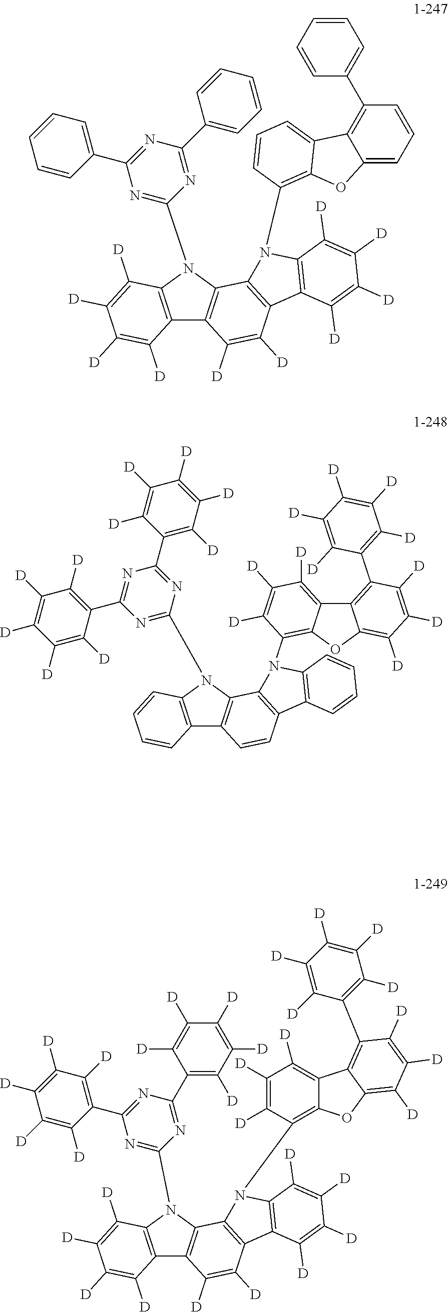

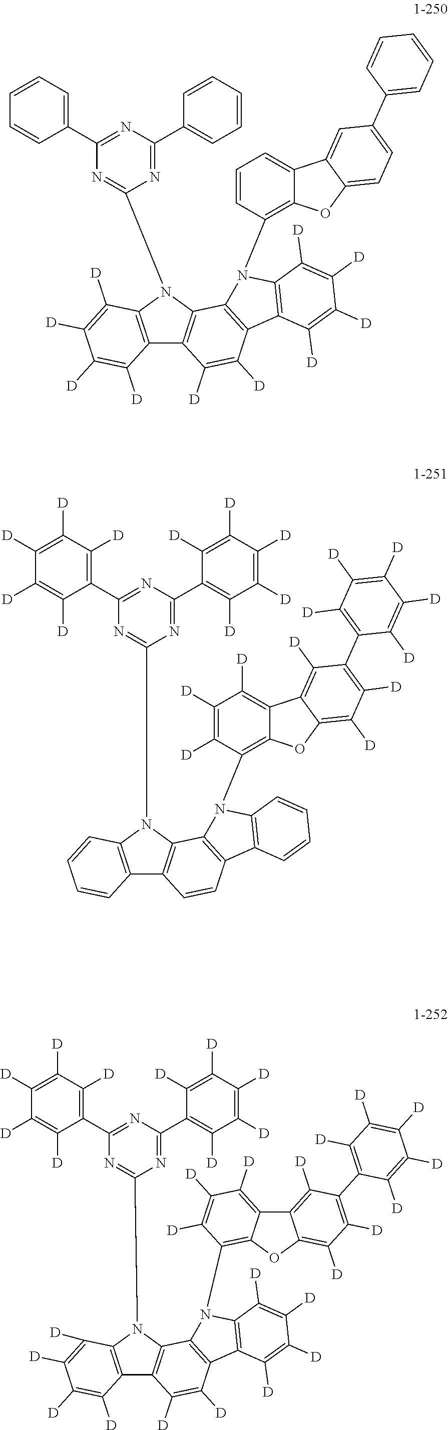

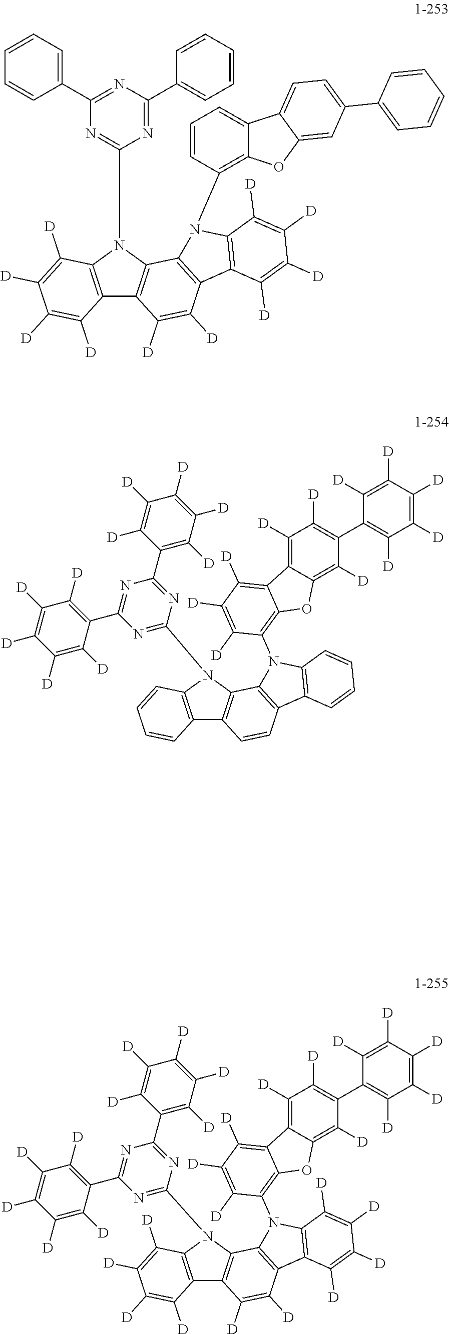

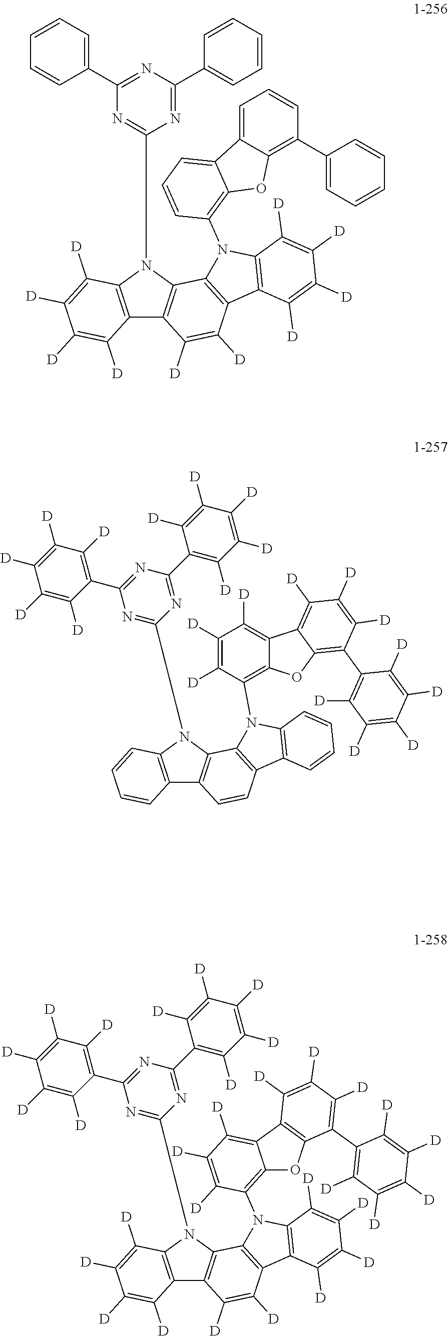

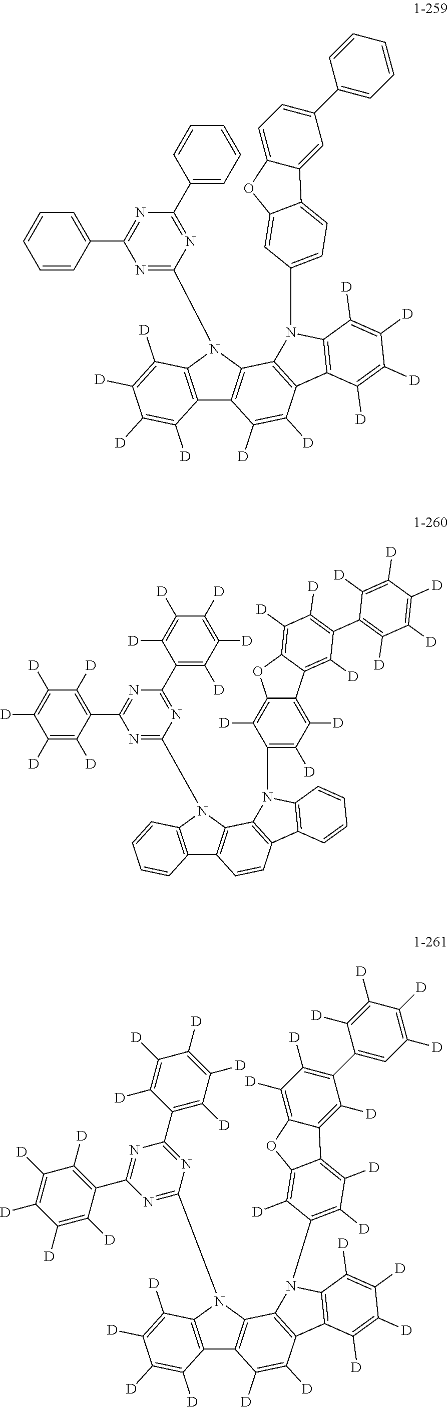

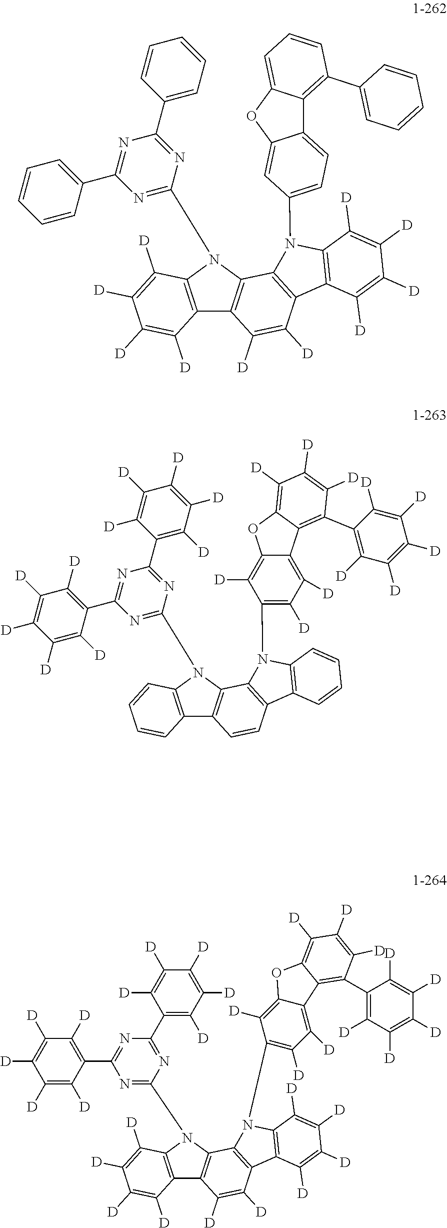

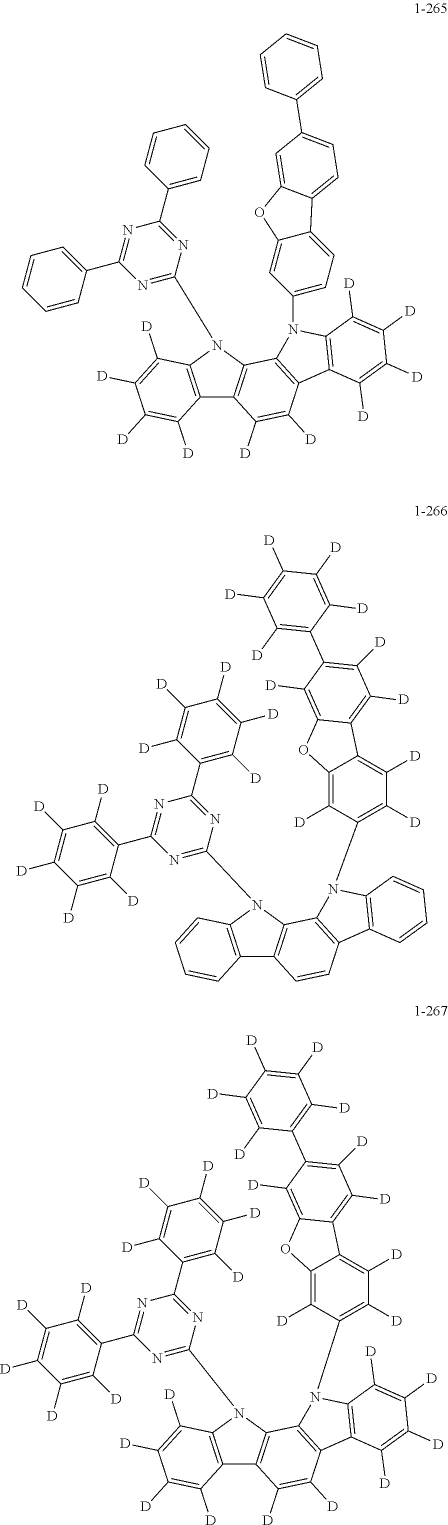

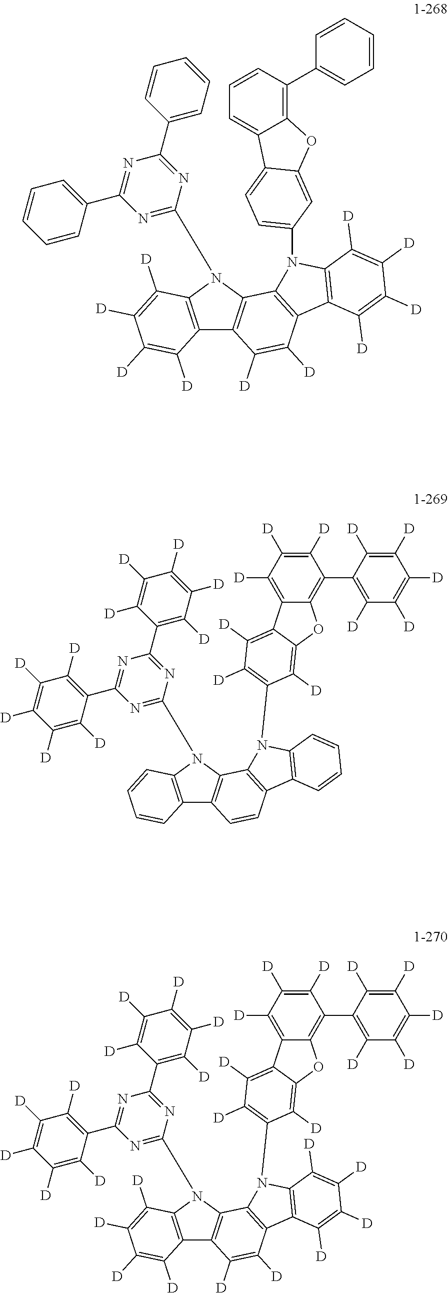

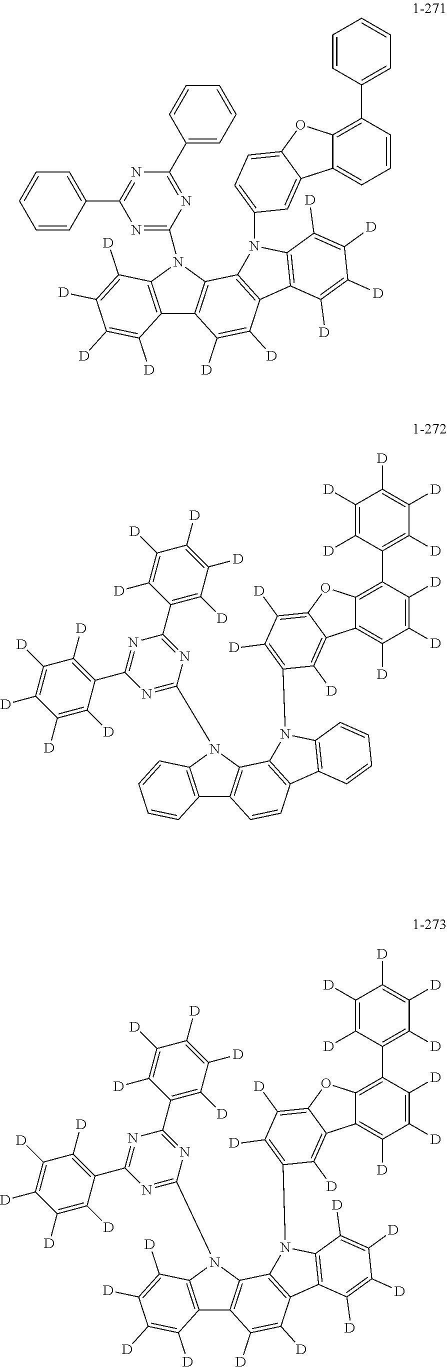

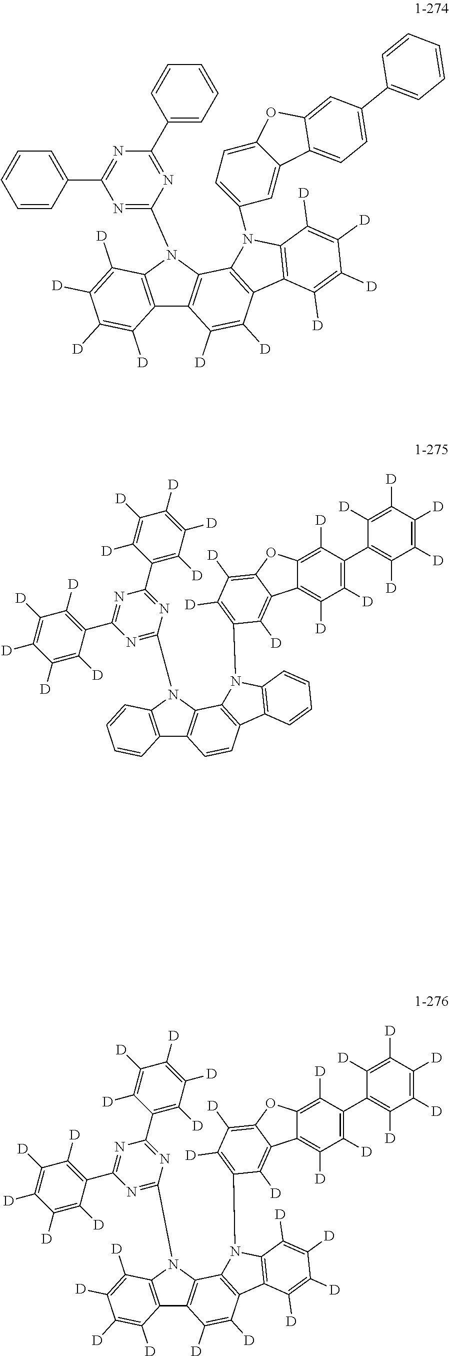

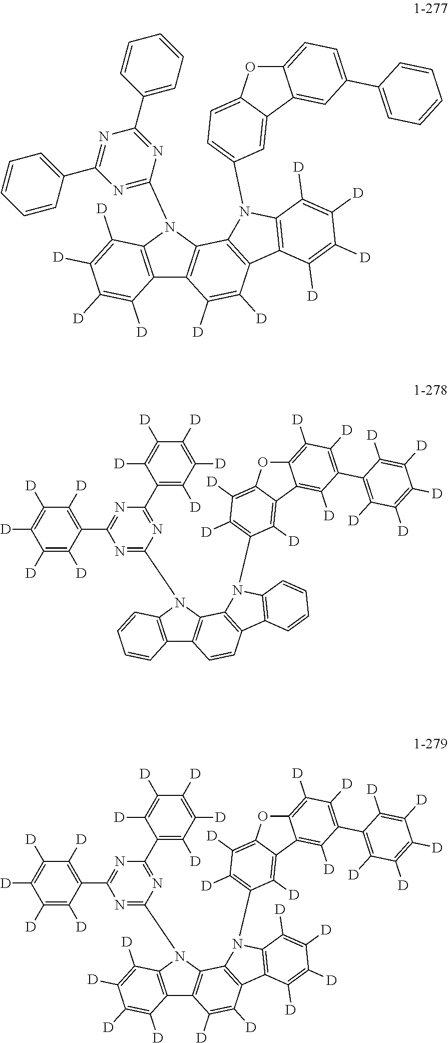

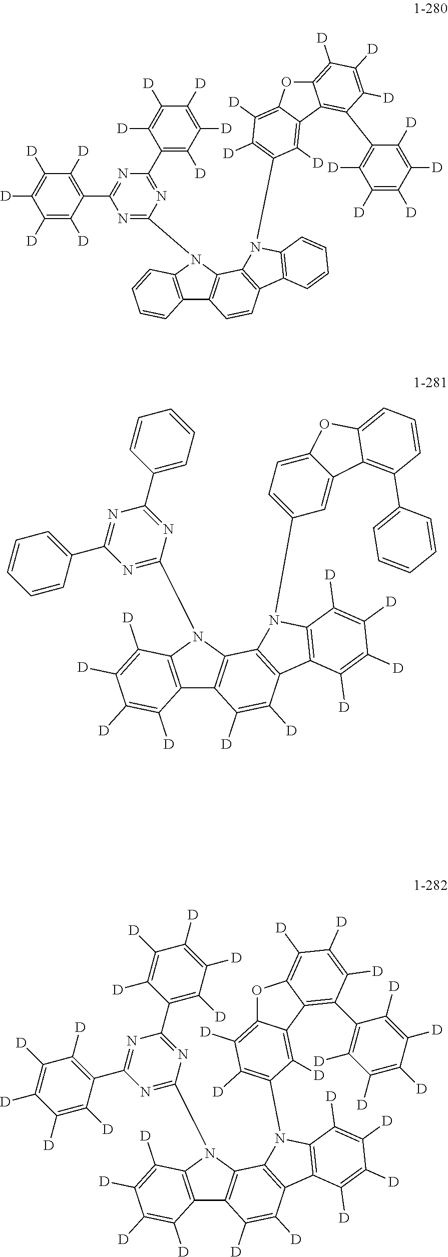

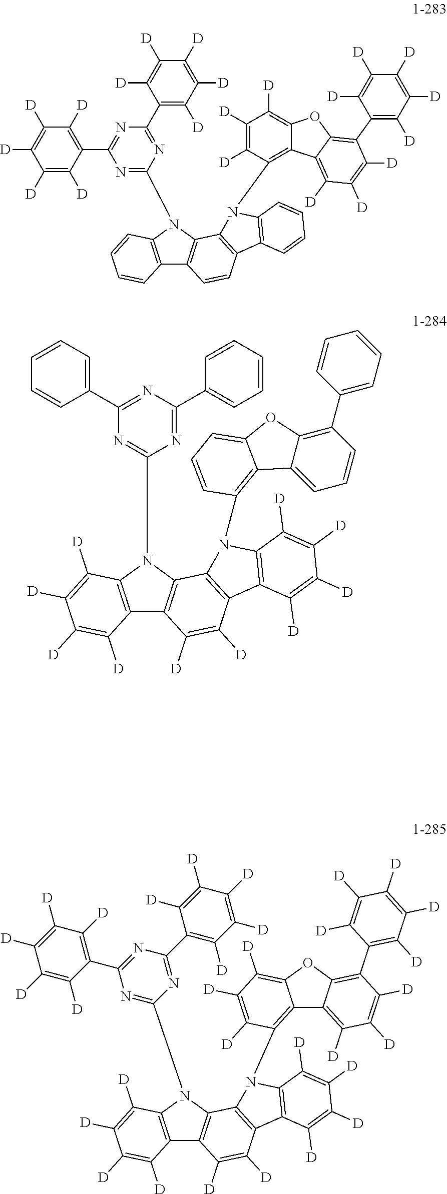

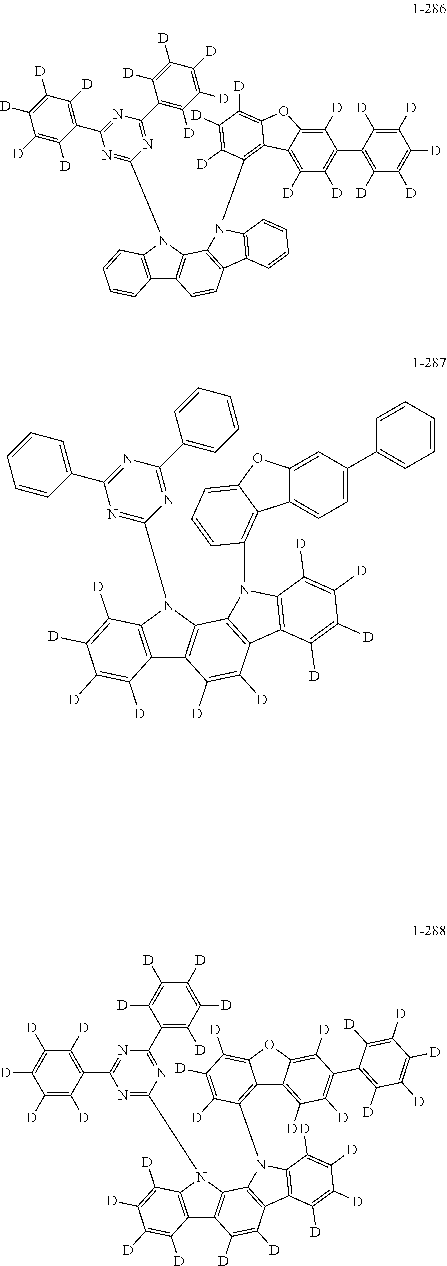

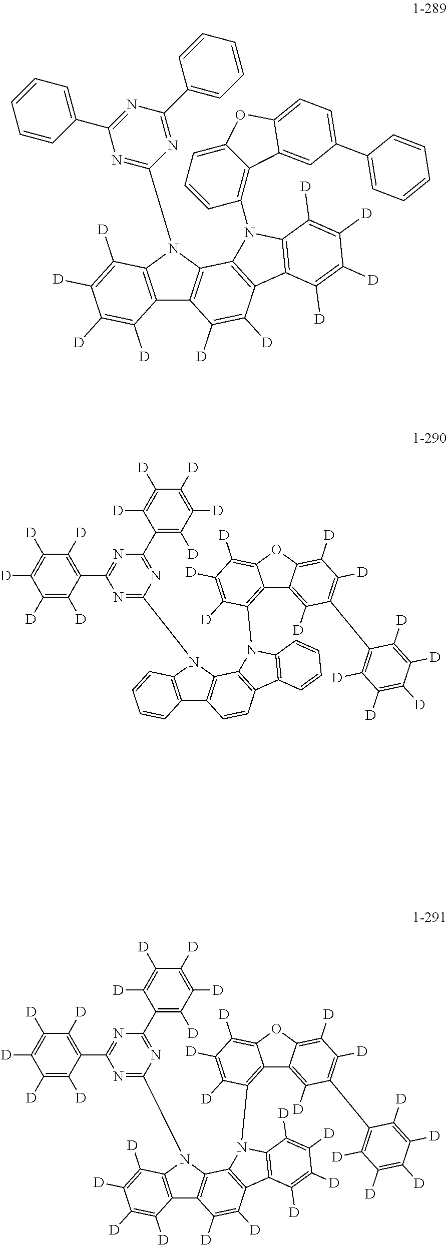

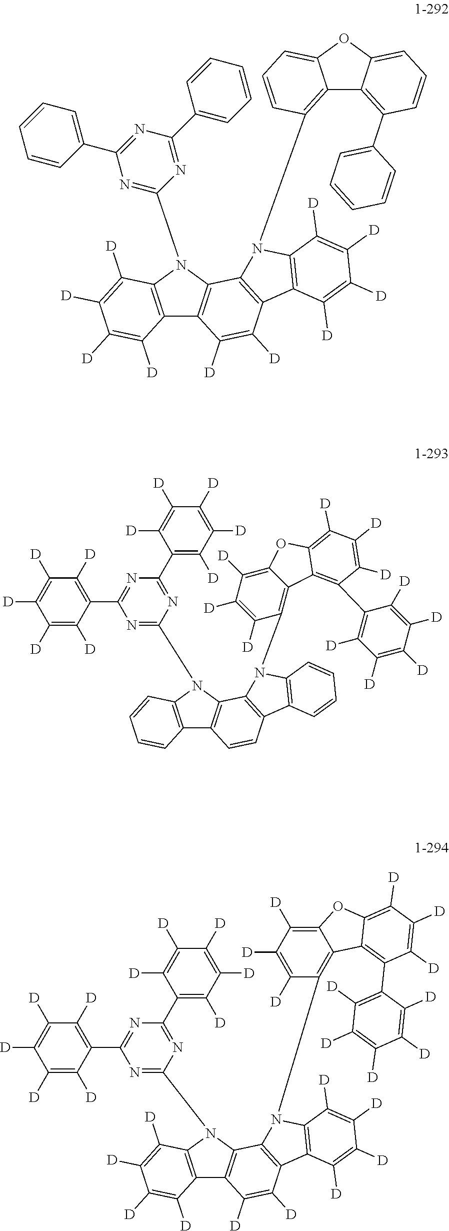

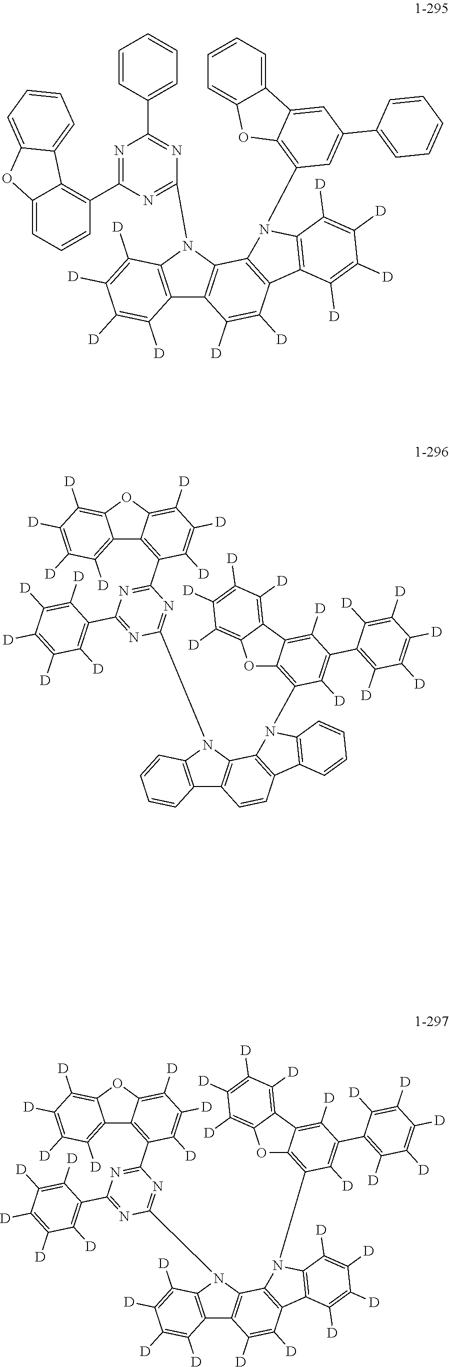

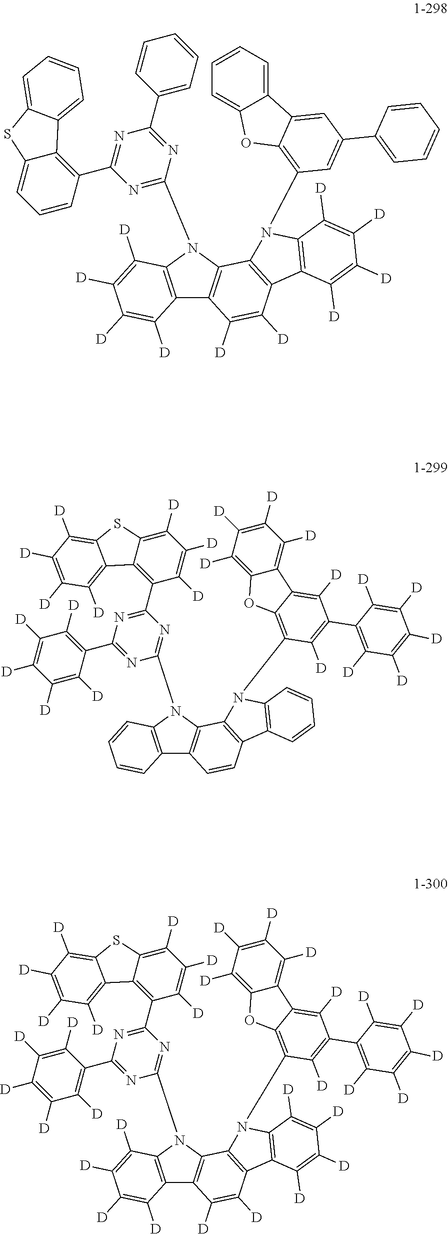

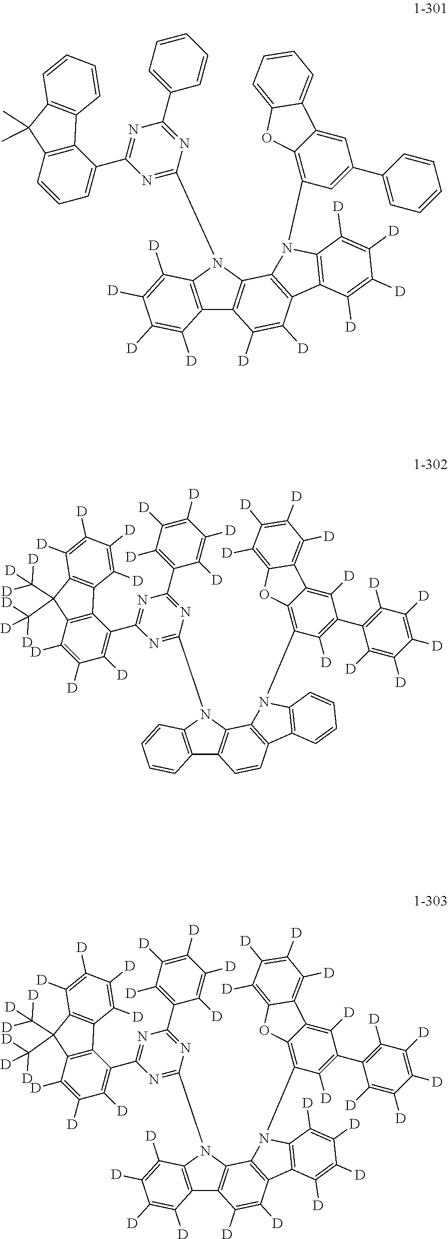

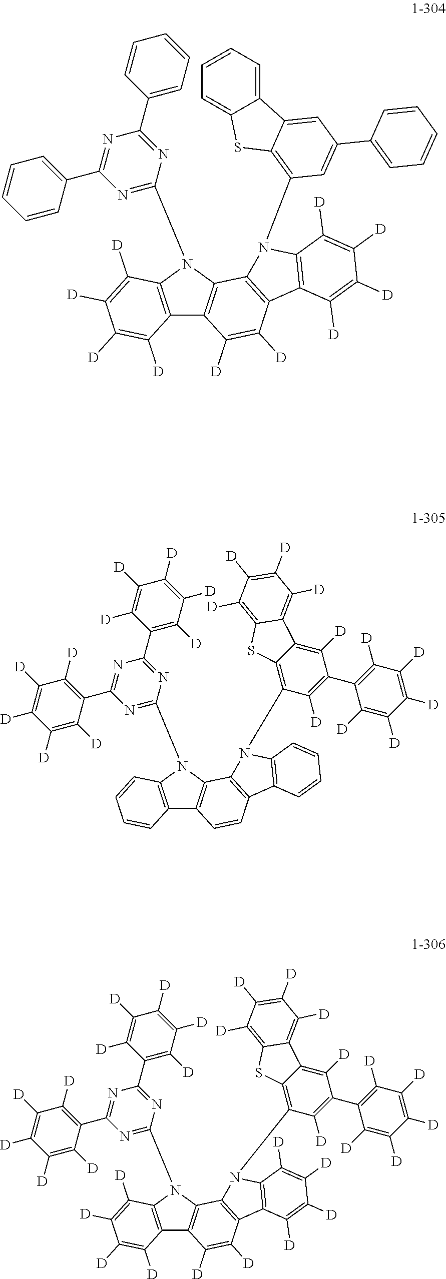

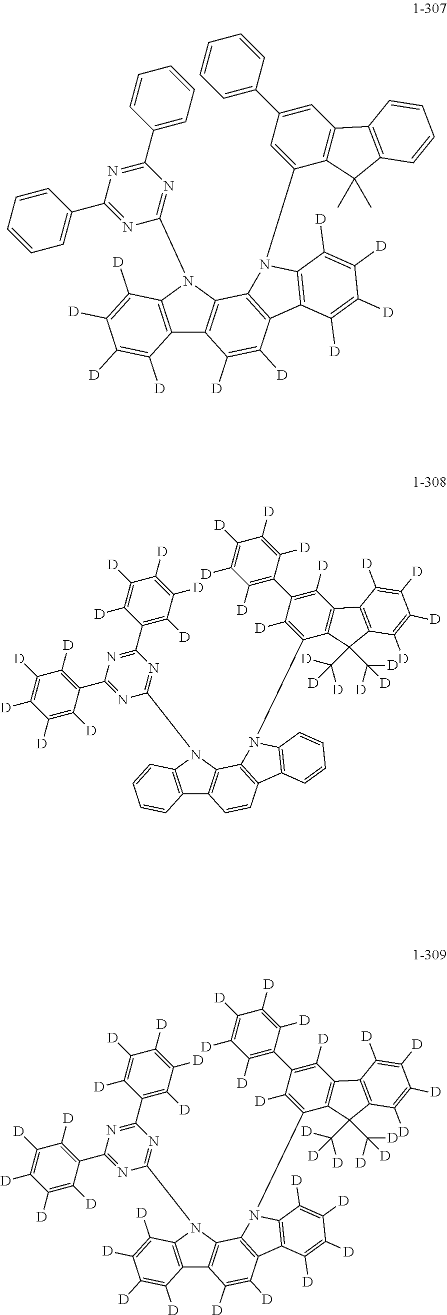

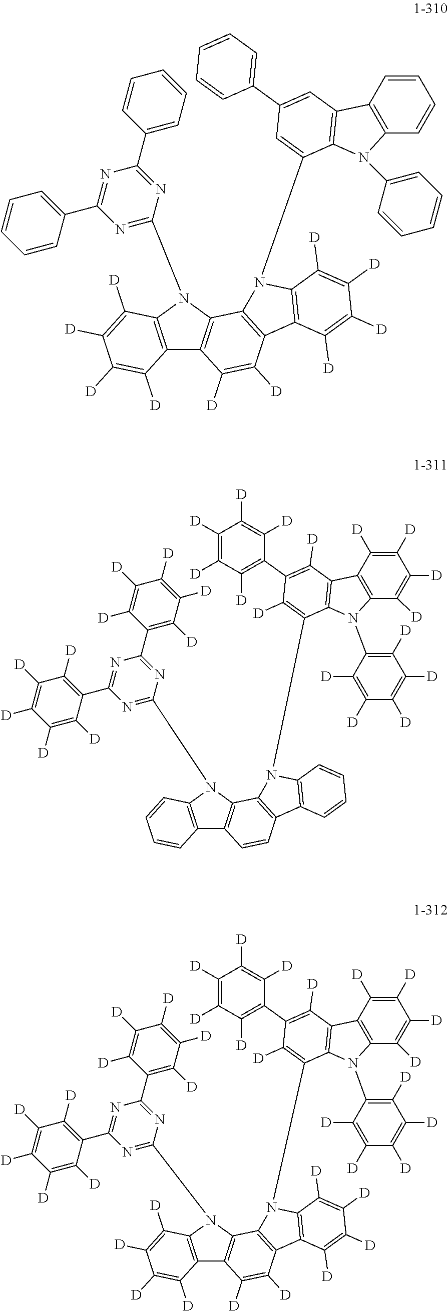

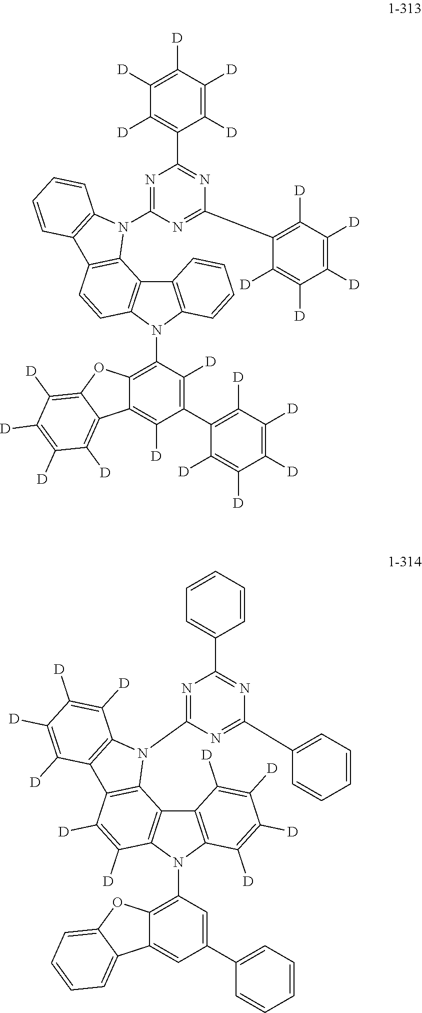 [SMILES: [2H]c1c([2H])c([2H])c(-c2nc(-c3c([2H])c([2H])c([2H])c([2H])c3[2H])nc(-n3c4ccccc4c4ccc5c(c6ccccc6n5-c5c([2H])c(-c6c([2H])c([2H])c([2H])c([2H])c6[2H])c([2H])c6c5oc5c([2H])c([2H])c([2H])c([2H])c56)c43)n2)c([2H])c1[2H] ; [2H]c1c([2H])c([2H])c2c(c1[2H])c1c(c([2H])c([2H])c3c4c([2H])c([2H])c([2H])c([2H])c4n(-c4nc(-c5ccccc5)nc(-c5ccccc5)n4)c31)n2-c1cc(-c2ccccc2)cc2c1oc1ccccc12]

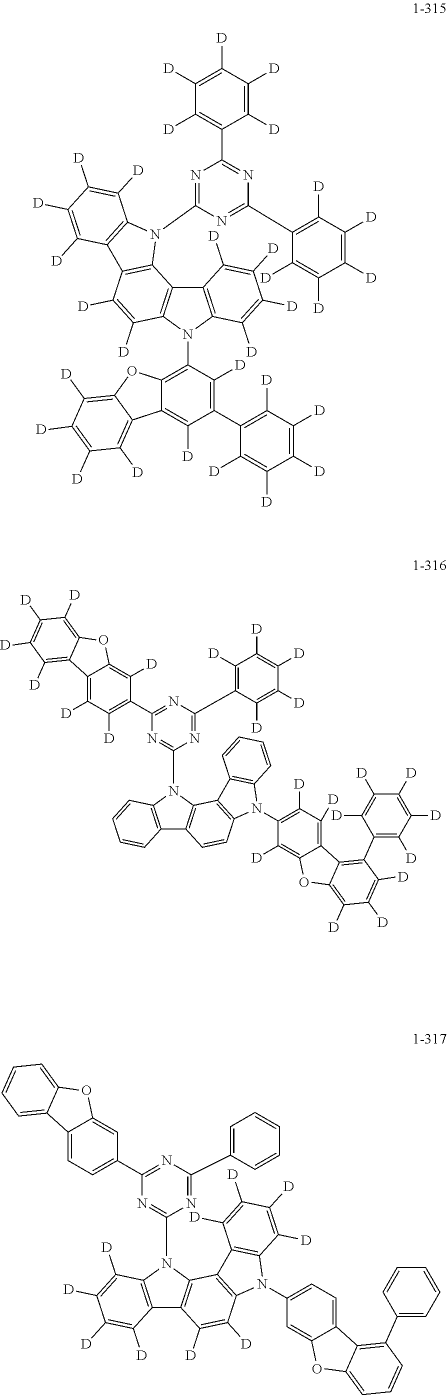

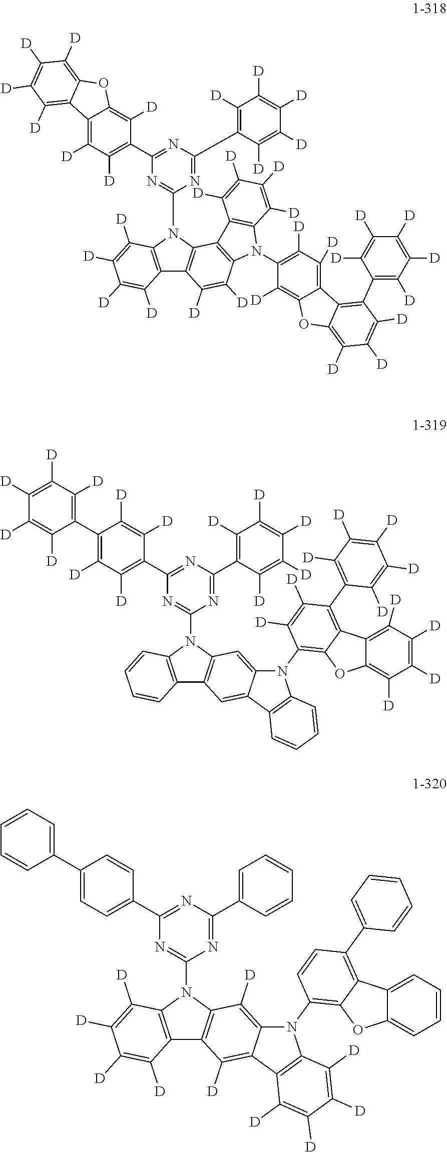

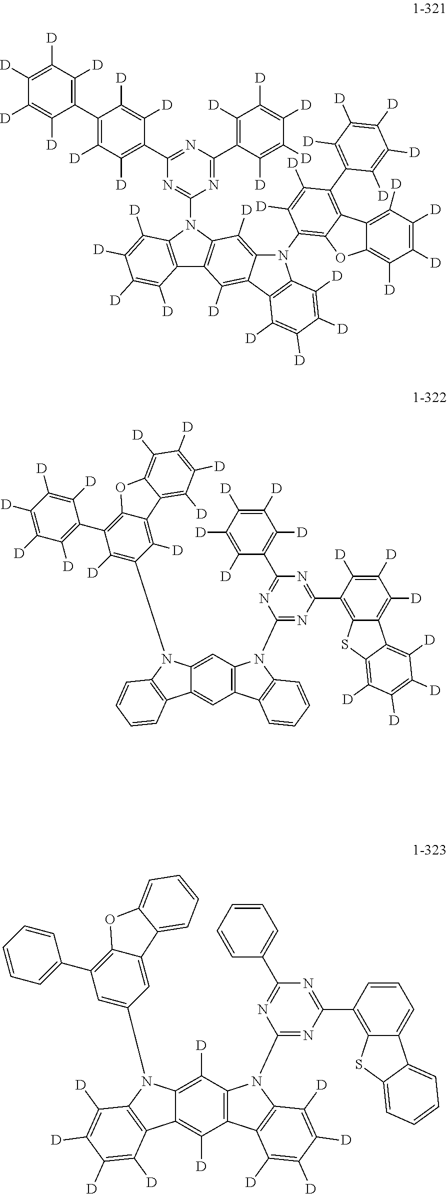

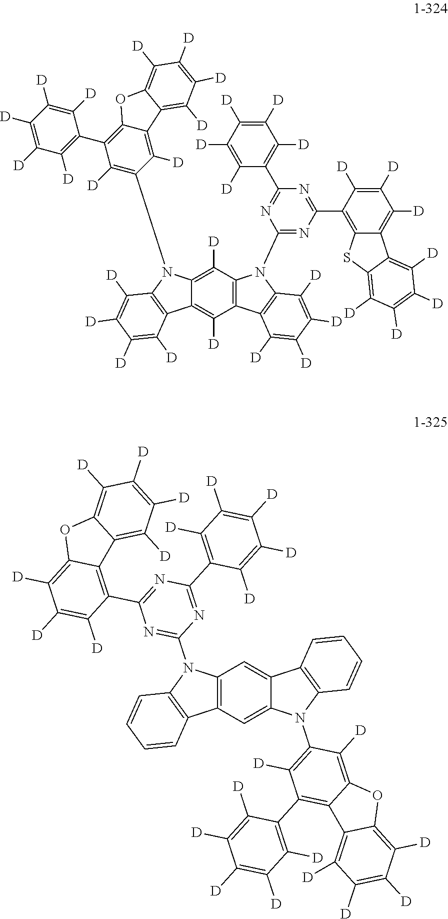

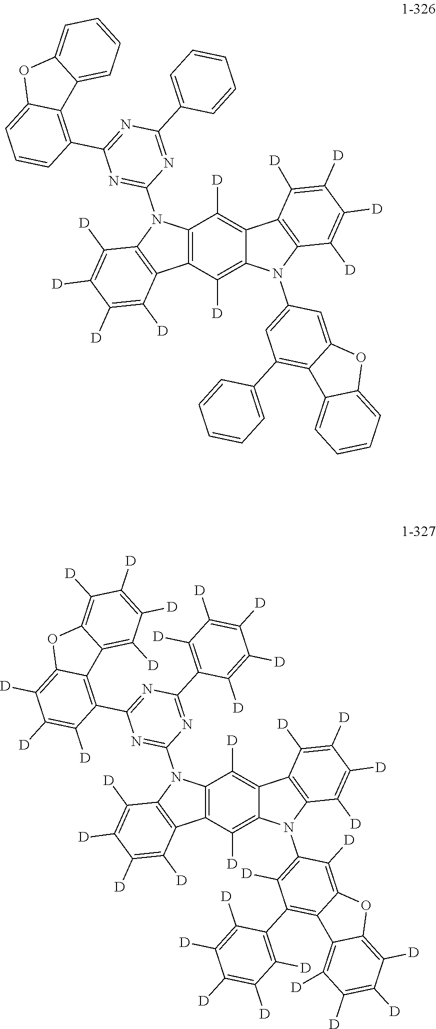 [SMILES: [2H]c1c([2H])c([2H])c2c(c1[2H])c1c([2H])c3c(c([2H])c1n2-c1cc(-c2ccccc2)c2c(c1)oc1ccccc12)c1c([2H])c([2H])c([2H])c([2H])c1n3-c1nc(-c2ccccc2)nc(-c2cccc3oc4ccccc4c23)n1 ; [2H]c1c([2H])c([2H])c(-c2nc(-c3c([2H])c([2H])c([2H])c4oc5c([2H])c([2H])c([2H])c([2H])c5c34)nc(-n3c4c([2H])c([2H])c([2H])c([2H])c4c4c([2H])c5c(c([2H])c43)c3c([2H])c([2H])c([2H])c([2H])c3n5-c3c([2H])c(-c4c([2H])c([2H])c([2H])c([2H])c4[2H])c4c(oc5c([2H])c([2H])c([2H])c([2H])c54)c3[2H])n2)c([2H])c1[2H]]

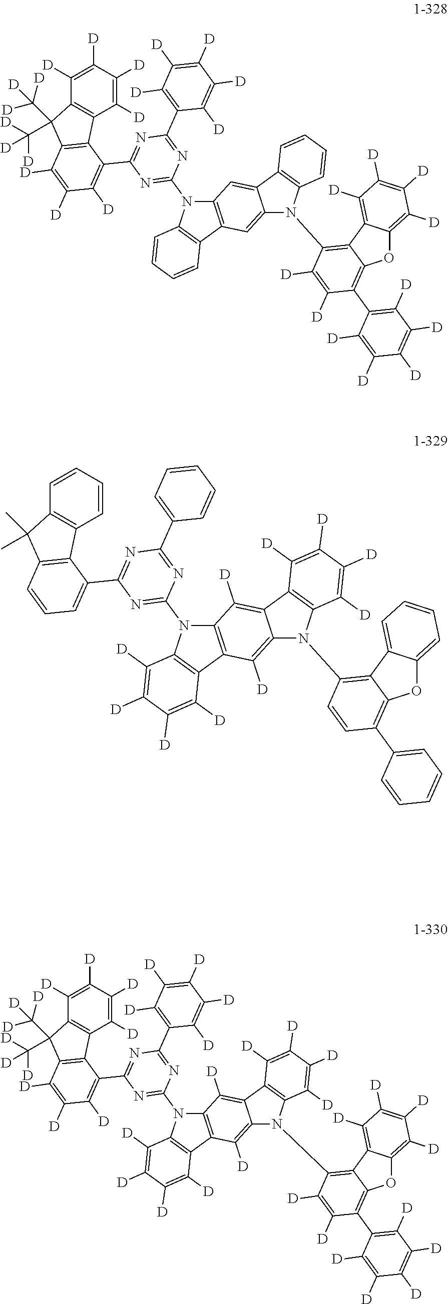

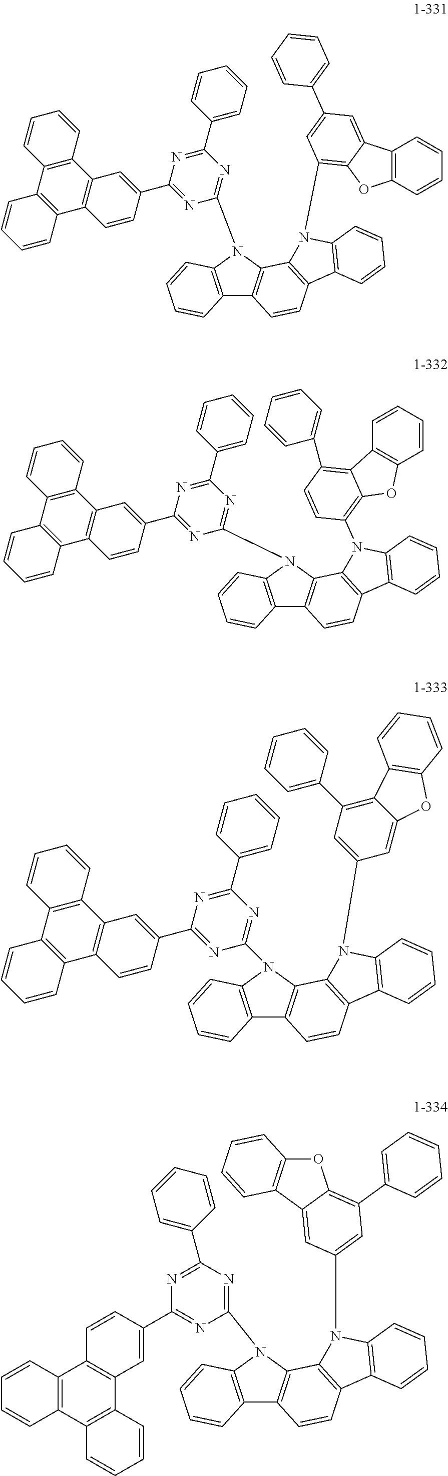 [SMILES: c1ccc(-c2cc(-n3c4ccccc4c4ccc5c6ccccc6n(-c6nc(-c7ccccc7)nc(-c7ccc8c9ccccc9c9ccccc9c8c7)n6)c5c43)c3oc4ccccc4c3c2)cc1 ; c1ccc(-c2nc(-c3ccc4c5ccccc5c5ccccc5c4c3)nc(-n3c4ccccc4c4ccc5c6ccccc6n(-c6ccc(-c7ccccc7)c7c6oc6ccccc67)c5c43)n2)cc1 ; c1ccc(-c2nc(-c3ccc4c5ccccc5c5ccccc5c4c3)nc(-n3c4ccccc4c4ccc5c6ccccc6n(-c6cc(-c7ccccc7)c7c(c6)oc6ccccc67)c5c43)n2)cc1 ; c1ccc(-c2nc(-c3ccc4c5ccccc5c5ccccc5c4c3)nc(-n3c4ccccc4c4ccc5c6ccccc6n(-c6cc(-c7ccccc7)c7oc8ccccc8c7c6)c5c43)n2)cc1]

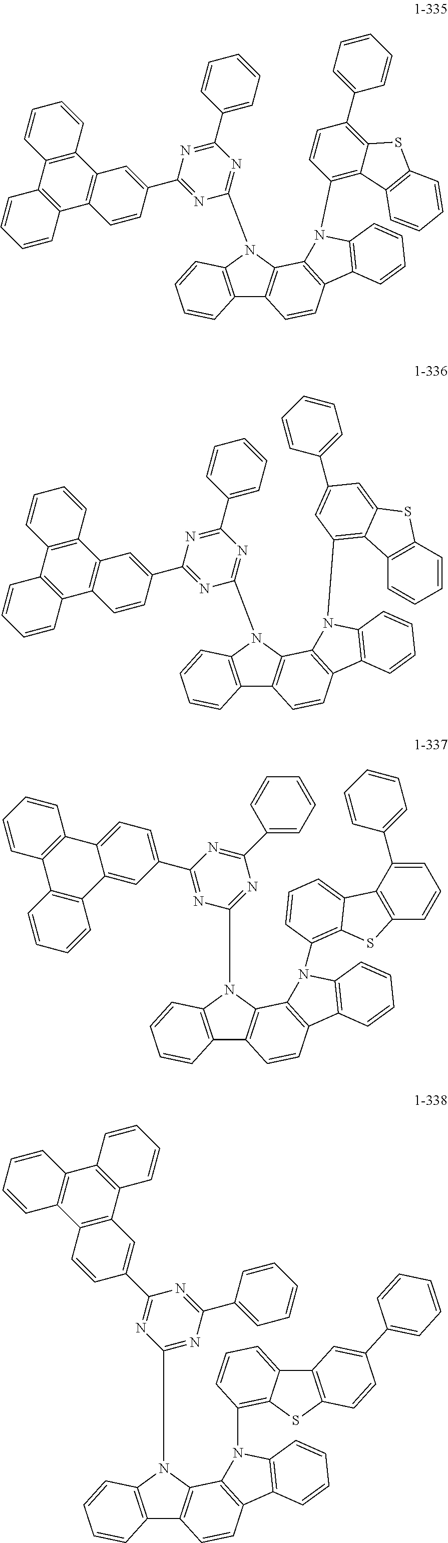 [SMILES: c1ccc(-c2nc(-c3ccc4c5ccccc5c5ccccc5c4c3)nc(-n3c4ccccc4c4ccc5c6ccccc6n(-c6ccc(-c7ccccc7)c7sc8ccccc8c67)c5c43)n2)cc1 ; c1ccc(-c2cc(-n3c4ccccc4c4ccc5c6ccccc6n(-c6nc(-c7ccccc7)nc(-c7ccc8c9ccccc9c9ccccc9c8c7)n6)c5c43)c3c(c2)sc2ccccc23)cc1 ; c1ccc(-c2nc(-c3ccc4c5ccccc5c5ccccc5c4c3)nc(-n3c4ccccc4c4ccc5c6ccccc6n(-c6cccc7c6sc6cccc(-c8ccccc8)c67)c5c43)n2)cc1 ; c1ccc(-c2ccc3sc4c(-n5c6ccccc6c6ccc7c8ccccc8n(-c8nc(-c9ccccc9)nc(-c9ccc%10c%11ccccc%11c%11ccccc%11c%10c9)n8)c7c65)cccc4c3c2)cc1]

In addition, by introducing various substituents to the structure of Chemical Formula 1, compounds having unique properties of the introduced substituents may be synthesized. For example, by introducing substituents normally used as a hole injection layer material, an electron blocking layer material, a hole transport layer material, a light emitting layer material, an electron transport layer material, a hole blocking layer material and a charge generation layer material used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials are enhanced, and material applications may become diverse.

In addition, one embodiment of the present disclosure relates to an organic light emitting device comprising:
a first electrode;
a second electrode provided opposite to the first electrode; and
one or more organic material layers provided between the first electrode and the second electrode,
wherein at least of the one or more layers of the organic material layers comprise the heterocyclic compound represented by Chemical Formula 1.

In one embodiment of the present disclosure, the first electrode may be a positive electrode, and the second electrode may be a negative electrode.

In another embodiment, the first electrode may be a negative electrode, and the second electrode may be a positive electrode.

In one embodiment of the present disclosure, the organic light emitting device may be a blue organic light emitting device, and the heterocyclic compound represented by Chemical Formula 1 may be used as a material of the blue organic light emitting device.

In another embodiment of the present disclosure, the organic light emitting device may be a green organic light emitting device, and the heterocyclic compound represented by Chemical Formula 1 may be used as a material of the green organic light emitting device.

In another embodiment of the present disclosure, the organic light emitting device may be a red organic light emitting device, and the heterocyclic compound represented by Chemical Formula 1 may be used as a material of the red organic light emitting device.

In one embodiment of the present disclosure, the organic light emitting device may be a blue organic light emitting device, and the heterocyclic compound represented by Chemical Formula 1 may be used as a light emitting layer material of the blue organic light emitting device.

In another embodiment of the present disclosure, the organic light emitting device may be a green organic light emitting device, and the heterocyclic compound represented by Chemical Formula 1 may be used as a light emitting layer material of the green organic light emitting device.

In another embodiment of the present disclosure, the organic light emitting device may be a red organic light emitting device, and the heterocyclic compound represented by Chemical Formula 1 may be used as a light emitting layer material of the red organic light emitting device.

Specific descriptions on the heterocyclic compound represented by Chemical Formula 1 are the same as the descriptions provided above.

The organic light emitting device of the present disclosure may be manufactured using common organic light emitting device manufacturing methods and materials except that one or more organic material layers are formed using the heterocyclic compound described above.

The heterocyclic compound may be formed into an organic material layer using a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, but may also be formed in a multilayer structure in which two or more organic material layers are laminated. For example, the organic light emitting device of the present disclosure may have a structure including a hole injection layer, an electron blocking layer, a hole transport layer, a light emitting layer, an electron transport layer, a hole blocking layer, an electron injection layer and the like as the organic material layer. However, the structure of the organic light emitting device is not limited thereto, and may include a smaller number of organic material layers.

In the organic light emitting device according to one embodiment of the present disclosure, the organic material layer comprising the heterocyclic compound represented by Chemical Formula 1 further comprises a heterocyclic compound represented by the following Chemical Formula 10.

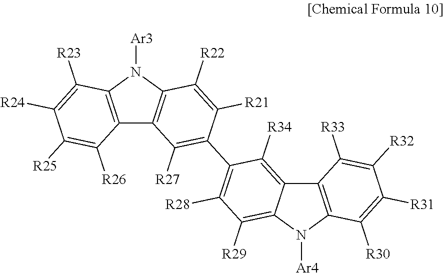

In Chemical Formula 10,
R21 to R34 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R301R302; —SiR301R302R303; and —NR301R302, or two or more groups adjacent to each other bond to each other to form a substituted or unsubstituted C6 to C60 aromatic hydrocarbon ring or a substituted or unsubstituted C2 to C60 heteroring, and R301, R302 and R303 are the same as or different from each other and each independently a substituted or unsubstituted C1 to C60 alkyl group; a substituted or unsubstituted C6 to C60 aryl group; or a substituted or unsubstituted C2 to C60 heteroaryl group, and
Ar3 and Ar4 are the same as or different from each other, and each independently a substituted or unsubstituted C6 to C60 aryl group; or a substituted or unsubstituted C2 to C60 heteroaryl group.

In one embodiment of the present disclosure, R21 to R34 are the same as or different from each other, and may be each independently hydrogen; deuterium; a substituted or unsubstituted C1 to C60 alkyl group; a substituted or unsubstituted C6 to C60 aryl group; or a substituted or unsubstituted C2 to C60 heteroaryl group.

In another embodiment of the present disclosure, R21 to R34 are the same as or different from each other, and may be each independently hydrogen; deuterium; a substituted or unsubstituted C1 to C30 alkyl group; a substituted or unsubstituted C6 to C30 aryl group; or a substituted or unsubstituted C2 to C30 heteroaryl group.

In another embodiment of the present disclosure, R21 to R34 are the same as or different from each other, and may be each independently hydrogen; deuterium; a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R21 to R34 are the same as or different from each other, and may be each independently hydrogen; or deuterium.

In one embodiment of the present disclosure, Ar3 and Ar4 are the same as or different from each other, and may be each independently a substituted or unsubstituted C6 to C30 aryl group; or a substituted or unsubstituted C2 to C30 heteroaryl group.

In another embodiment of the present disclosure, Ar3 and Ar4 are the same as or different from each other, and may be each independently a substituted or unsubstituted C6 to C20 aryl group; or a substituted or unsubstituted C2 to C20 heteroaryl group.

In another embodiment of the present disclosure, Ar3 and Ar4 are the same as or different from each other, and may be each independently a substituted or unsubstituted phenyl group; a substituted or unsubstituted biphenyl group; a substituted or unsubstituted terphenyl group; a substituted or unsubstituted naphthyl group; a substituted or unsubstituted fluorenyl group; a substituted or unsubstituted spirofluorenyl group; a substituted or unsubstituted triphenylenyl group; a substituted or unsubstituted dibenzofuranyl group; or a substituted or unsubstituted dibenzothiophenyl group.

In one embodiment of the present disclosure, the compound represented by Chemical Formula 10 may not include deuterium as a substituent, or a content of deuterium may be greater than 0%, 1% or greater, 10% or greater, 20% or greater, 30% or greater, 40% or greater or 50% or greater, and may be 100% or less, 90% or less, 80% or less, 70% or less or 60% or less based on the total number of hydrogen atoms and deuterium atoms.

In another embodiment of the present disclosure, the compound represented by Chemical Formula 10 may not include deuterium as a substituent, or a content of deuterium may be from 1% to 100% based on the total number of hydrogen atoms and deuterium atoms.

In another embodiment of the present disclosure, the compound represented by Chemical Formula 10 may not include deuterium as a substituent, or a content of deuterium may be from 20% to 90% based on the total number of hydrogen atoms and deuterium atoms.

In another embodiment of the present disclosure, the compound represented by Chemical Formula 10 may not include deuterium as a substituent, or a content of deuterium may be from 30% to 80% based on the total number of hydrogen atoms and deuterium atoms.

In another embodiment of the present disclosure, the compound represented by Chemical Formula 10 may not include deuterium as a substituent, or a content of deuterium may be from 50% to 70% based on the total number of hydrogen atoms and deuterium atoms.

When including the compound represented by Chemical Formula 1 and the compound represented by Chemical Formula 10 at the same time, effects of more superior efficiency and lifetime are obtained. This may lead to a forecast that an exciplex phenomenon occurs when including the two compounds at the same time.

The exciplex phenomenon is a phenomenon of releasing energy having sizes of a donor (p-host) HOMO energy level and an acceptor (n-host) LUMO energy level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transport ability and an acceptor (n-host) having a favorable electron transport ability are used as a host of a light emitting layer, holes are injected to the p-host and electrons are injected to the n-host, and a driving voltage may be lowered, which resultantly helps with enhancement in the lifetime. In other words, when using the compound represented by Chemical Formula 1 as the acceptor and using the compound represented by Chemical Formula 10 as the donor, excellent device properties are obtained.

In one embodiment of the present disclosure, when comprising the heterocyclic compound represented by Chemical Formula 1 and the heterocyclic compound represented by Chemical Formula 10 at the same time, at least one of the compounds does not include deuterium as a substituent, or a content of deuterium may be greater than 0%, 1% or greater, 10% or greater, 20% or greater, 30% or greater, 40% or greater or 50% or greater, and may be 100% or less, 90% or less, 80% or less, 70% or less or 60% or less based on the total number of hydrogen atoms and deuterium atoms.

In another embodiment of the present disclosure, when comprising the heterocyclic compound represented by Chemical Formula 1 and the heterocyclic compound represented by Chemical Formula 10 at the same time, at least one of the compounds does not include deuterium as a substituent, or a content of deuterium may be from 1% to 100% based on the total number of hydrogen atoms and deuterium atoms.

In another embodiment of the present disclosure, when comprising the heterocyclic compound represented by Chemical Formula 1 and the heterocyclic compound represented by Chemical Formula 10 at the same time, at least one of the compounds does not include deuterium as a substituent, or a content of deuterium may be from 20% to 90% based on the total number of hydrogen atoms and deuterium atoms.

In another embodiment of the present disclosure, when comprising the heterocyclic compound represented by Chemical Formula 1 and the heterocyclic compound represented by Chemical Formula 10 at the same time, at least one of the compounds does not include deuterium as a substituent, or a content of deuterium may be from 30% to 80% based on the total number of hydrogen atoms and deuterium atoms.

In another embodiment of the present disclosure, when comprising the heterocyclic compound represented by Chemical Formula 1 and the heterocyclic compound represented by Chemical Formula 10 at the same time, at least one of the compounds does not include deuterium as a substituent, or a content of deuterium may be from 50% to 70% based on the total number of hydrogen atoms and deuterium atoms.

In one embodiment of the present disclosure, the heterocyclic compound represented by Chemical Formula 10 may be any one selected from among the following compounds.

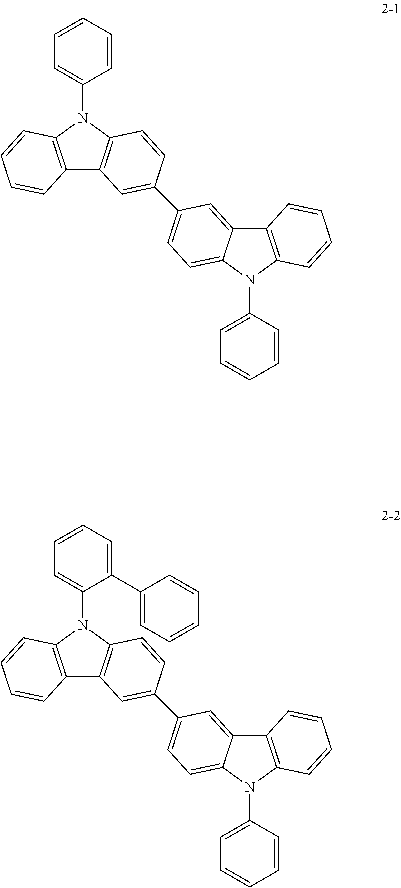 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1]

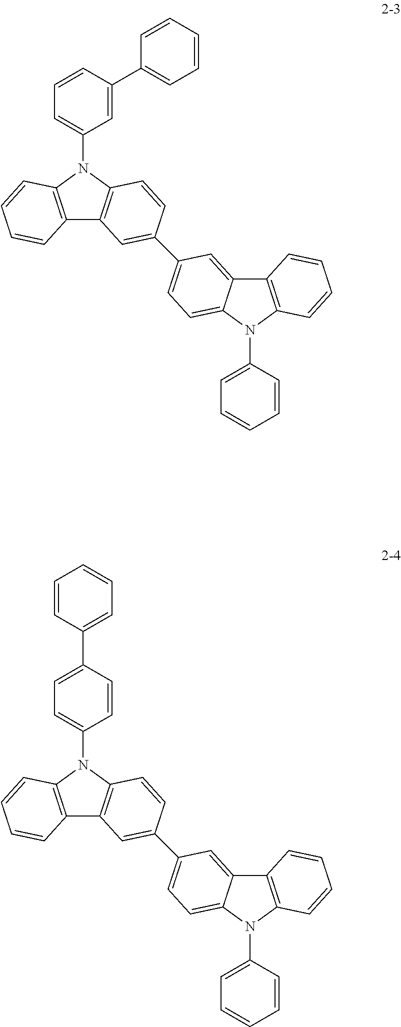 [SMILES: c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1]

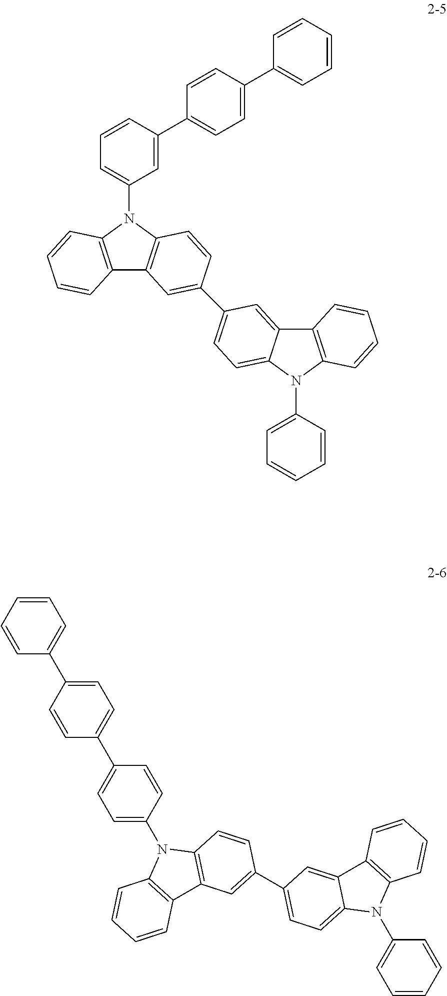 [SMILES: c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

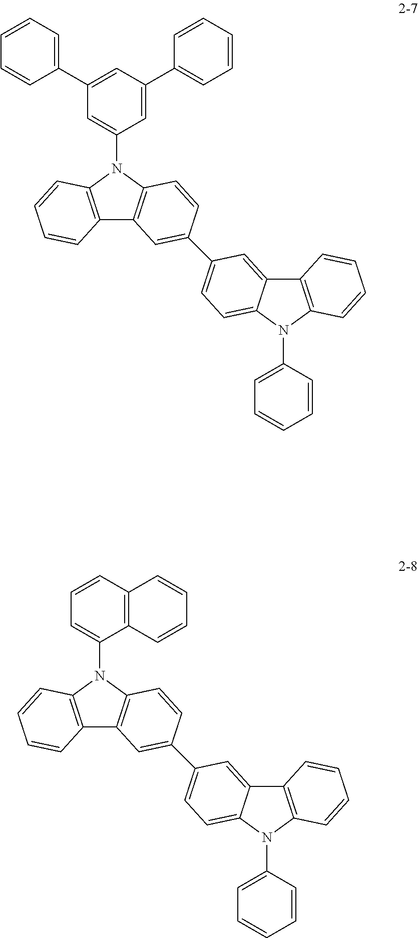 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1]

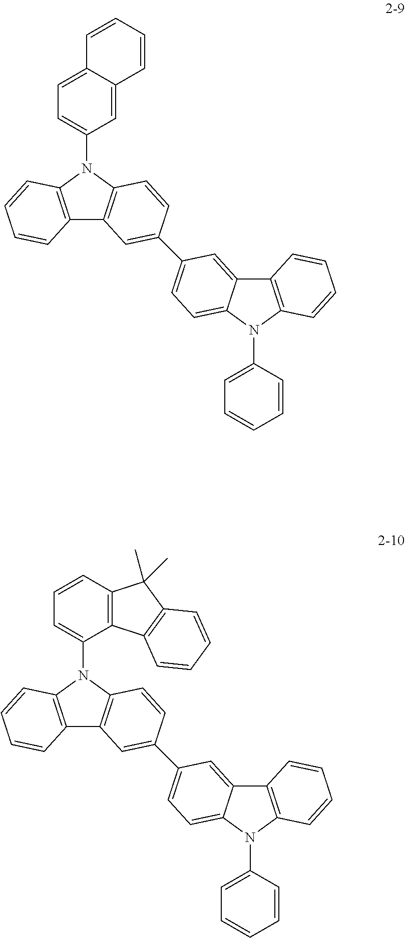 [SMILES: c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21]

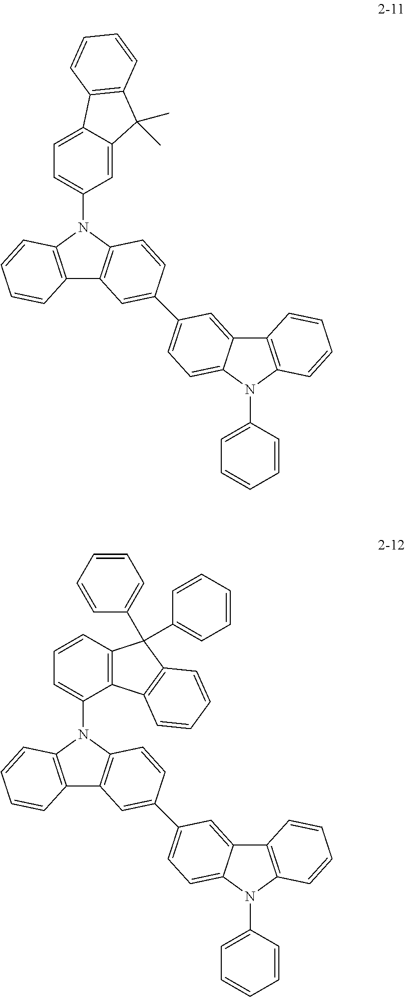 [SMILES: CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

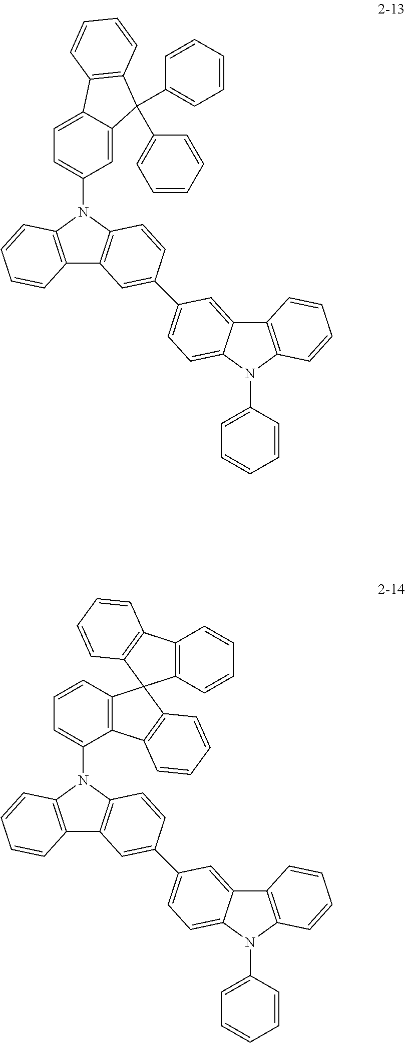 [SMILES: c1ccc(-n2c3ccccc3c3cc(-c4ccc5c(c4)c4ccccc4n5-c4ccc5c(c4)C(c4ccccc4)(c4ccccc4)c4ccccc4-5)ccc32)cc1 ; c1ccc(-n2c3ccccc3c3cc(-c4ccc5c(c4)c4ccccc4n5-c4cccc5c4-c4ccccc4C54c5ccccc5-c5ccccc54)ccc32)cc1]

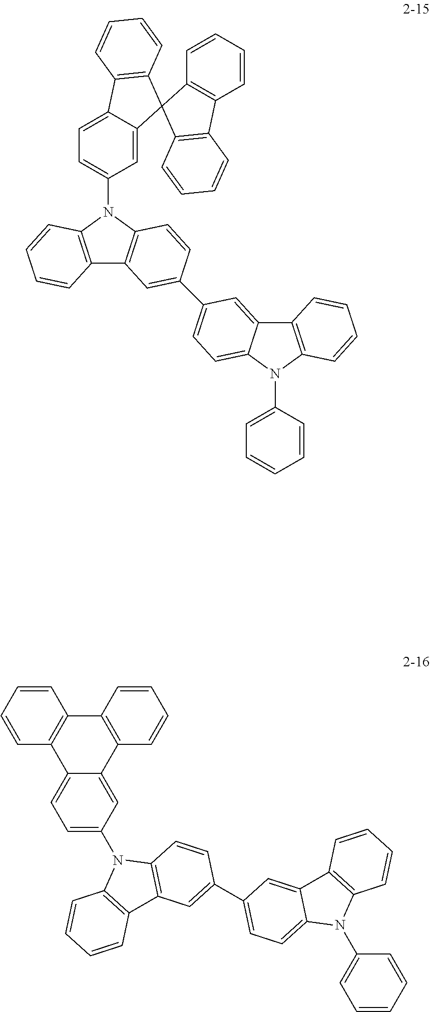 [SMILES: c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1 ; c1ccc(-n2c3ccccc3c3cc(-c4ccc5c(c4)c4ccccc4n5-c4ccc5c6ccccc6c6ccccc6c5c4)ccc32)cc1]

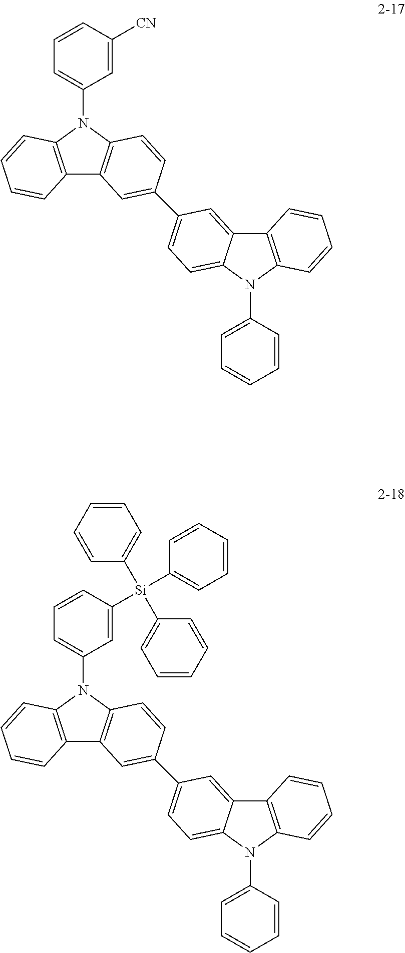 [SMILES: N#Cc1cccc(-n2c3ccccc3c3cc(-c4ccc5c(c4)c4ccccc4n5-c4ccccc4)ccc32)c1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1]

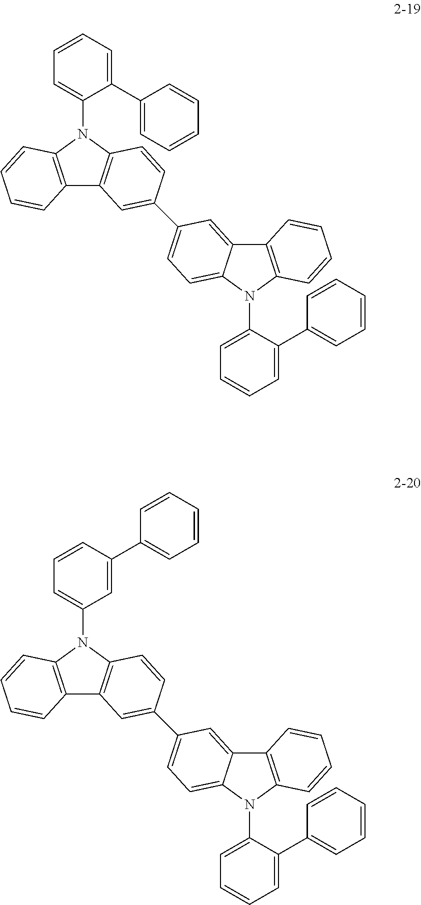 [SMILES: c1ccc(-c2ccccc2-n2c3ccccc3c3cc(-c4ccc5c(c4)c4ccccc4n5-c4ccccc4-c4ccccc4)ccc32)cc1 ; c1ccc(-c2cccc(-n3c4ccccc4c4cc(-c5ccc6c(c5)c5ccccc5n6-c5ccccc5-c5ccccc5)ccc43)c2)cc1]

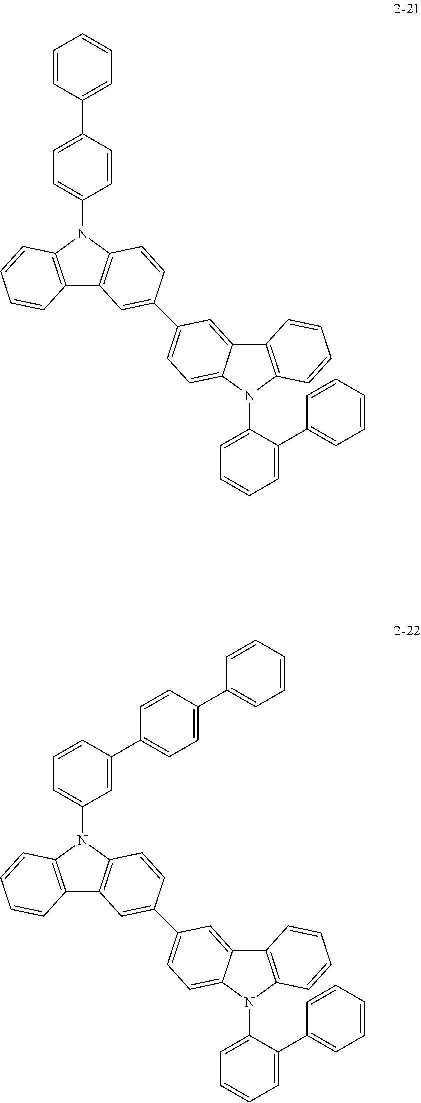 [SMILES: c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2ccc(-c3cccc(-n4c5ccccc5c5cc(-c6ccc7c(c6)c6ccccc6n7-c6ccccc6-c6ccccc6)ccc54)c3)cc2)cc1]

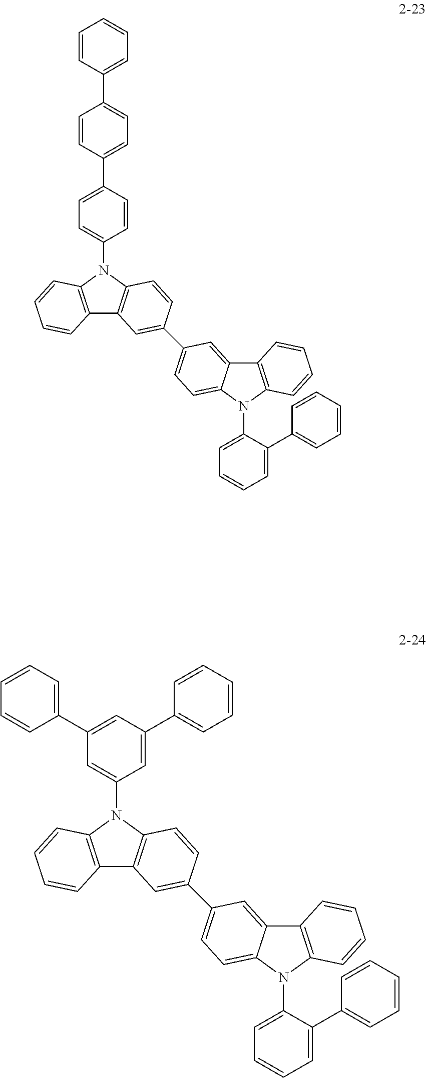 [SMILES: c1ccc(-c2ccc(-c3ccc(-n4c5ccccc5c5cc(-c6ccc7c(c6)c6ccccc6n7-c6ccccc6-c6ccccc6)ccc54)cc3)cc2)cc1 ; c1ccc(-c2cc(-c3ccccc3)cc(-n3c4ccccc4c4cc(-c5ccc6c(c5)c5ccccc5n6-c5ccccc5-c5ccccc5)ccc43)c2)cc1]

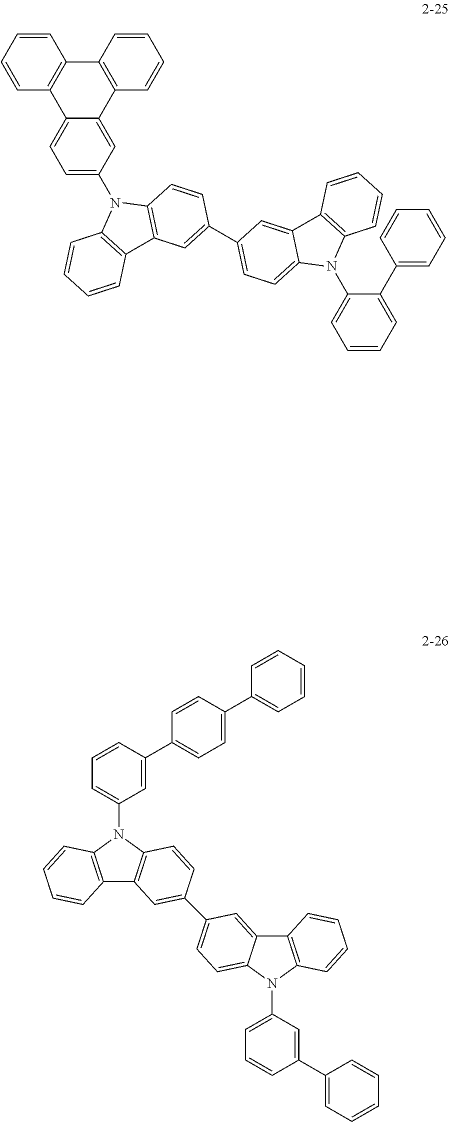 [SMILES: c1ccc(-c2ccccc2-n2c3ccccc3c3cc(-c4ccc5c(c4)c4ccccc4n5-c4ccc5c6ccccc6c6ccccc6c5c4)ccc32)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1]

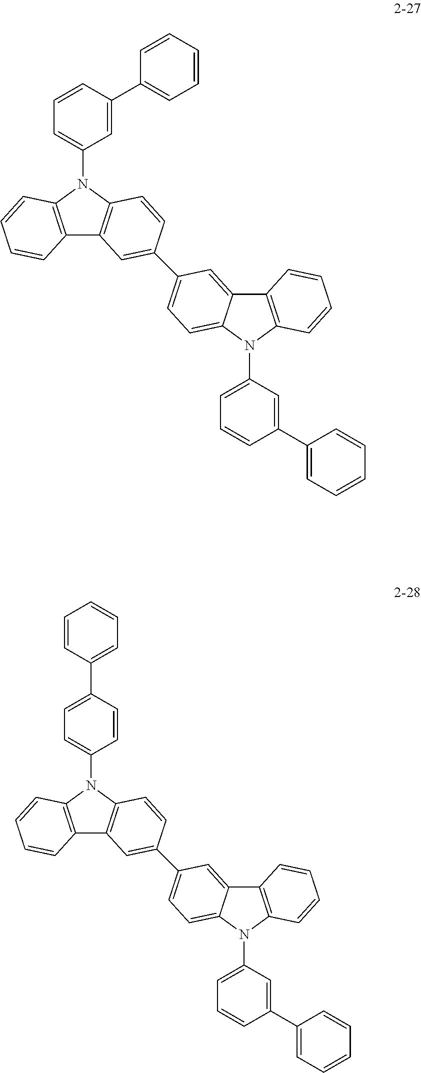 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

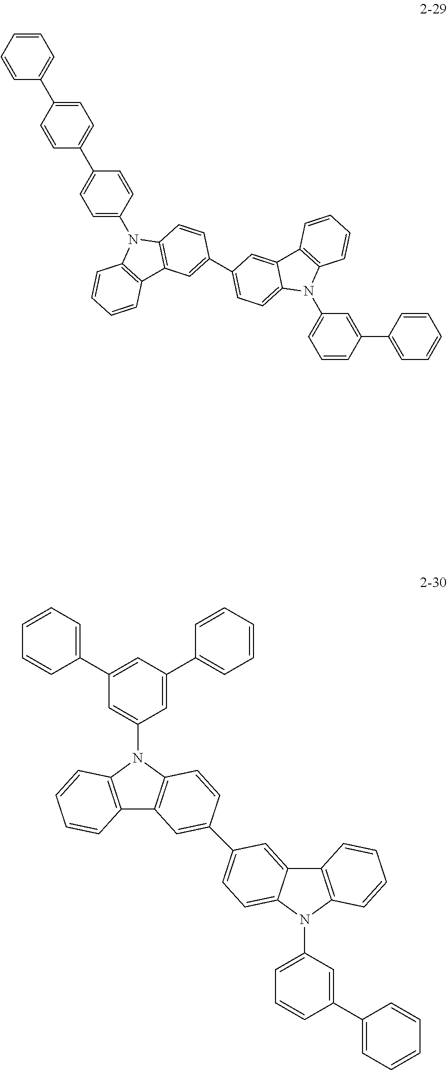 [SMILES: c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

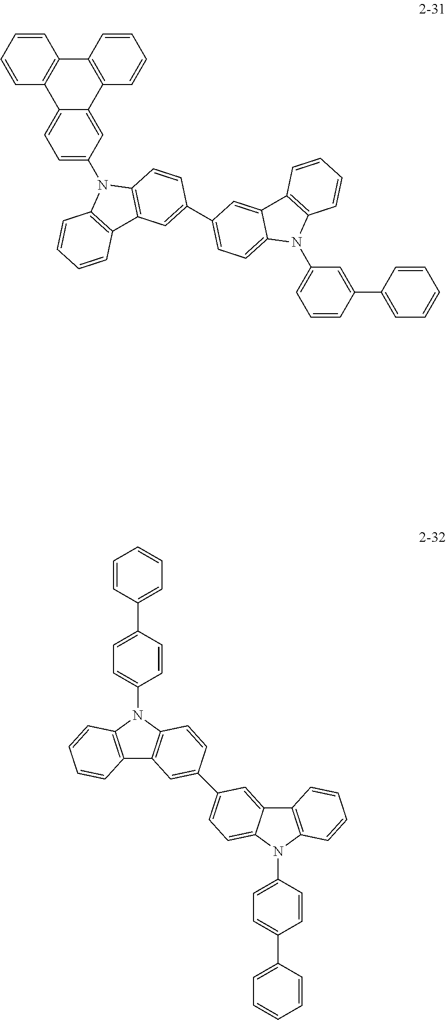 [SMILES: c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

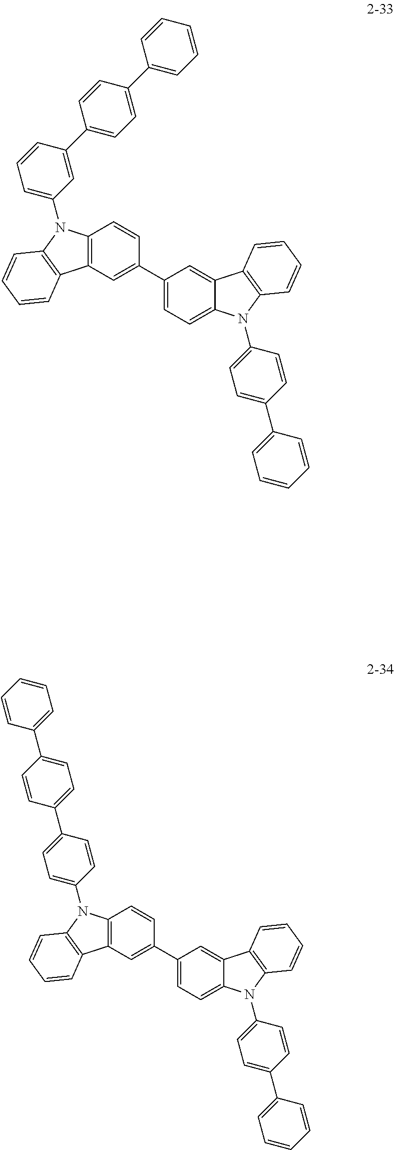 [SMILES: c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1]

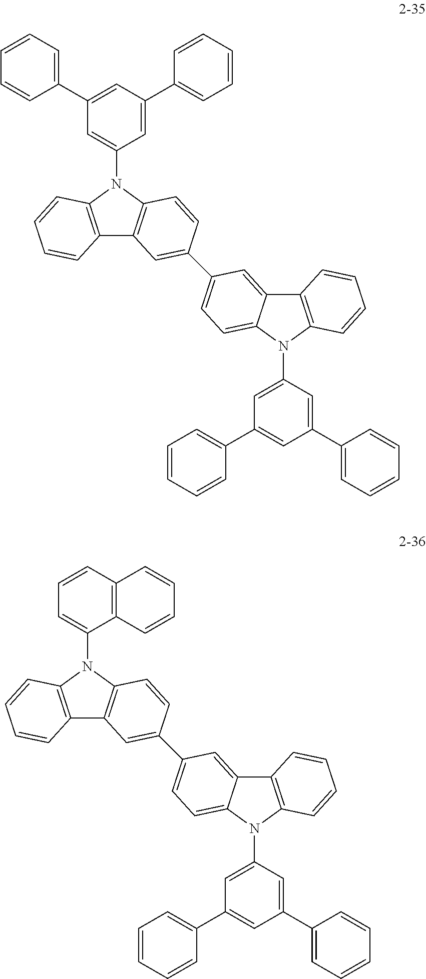 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5cccc6ccccc56)ccc43)c2)cc1]

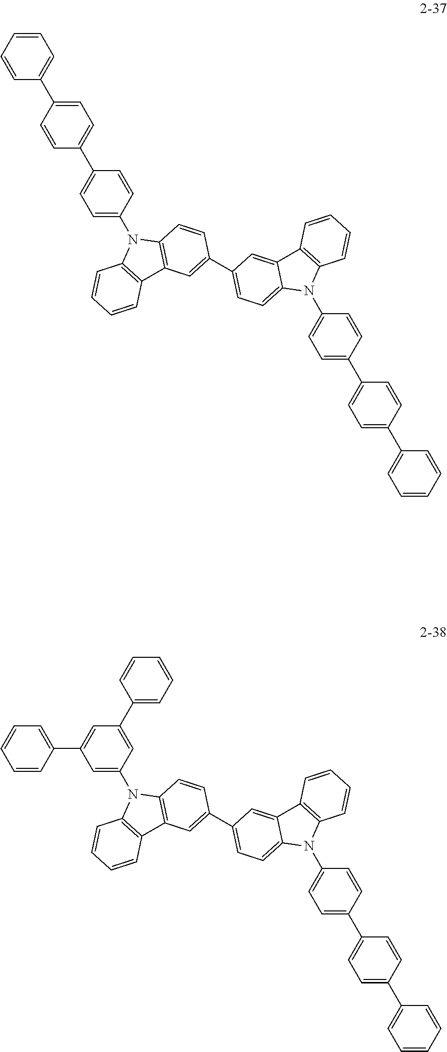 [SMILES: c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1]

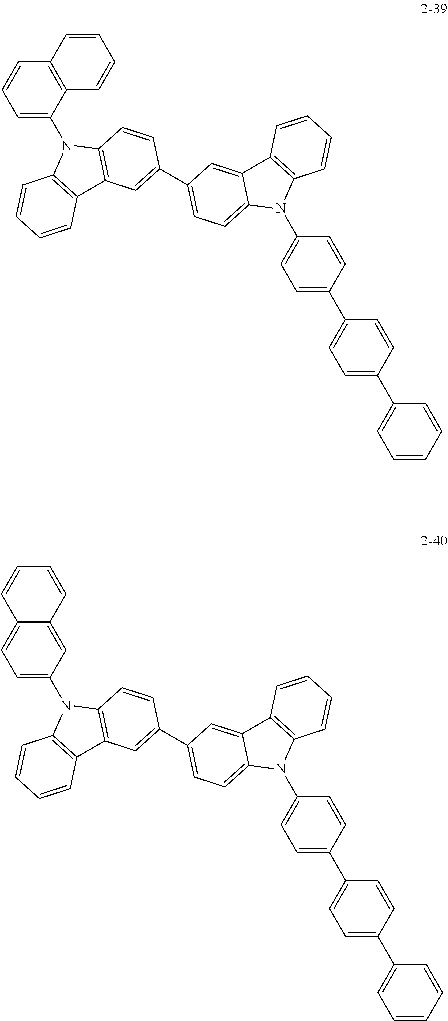 [SMILES: c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1]

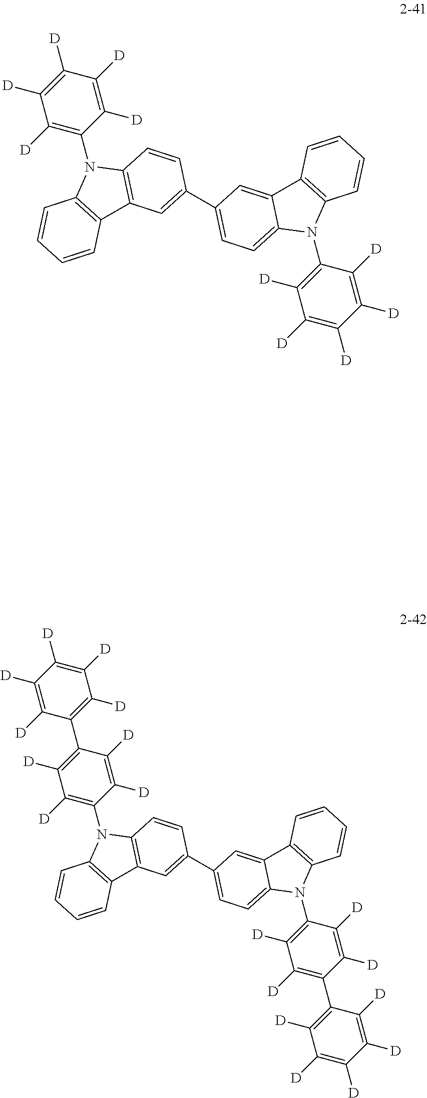 [SMILES: [2H]c1c([2H])c([2H])c(-n2c3ccccc3c3cc(-c4ccc5c(c4)c4ccccc4n5-c4c([2H])c([2H])c([2H])c([2H])c4[2H])ccc32)c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c([2H])c6[2H])c([2H])c5[2H])ccc43)c([2H])c2[2H])c([2H])c1[2H]]

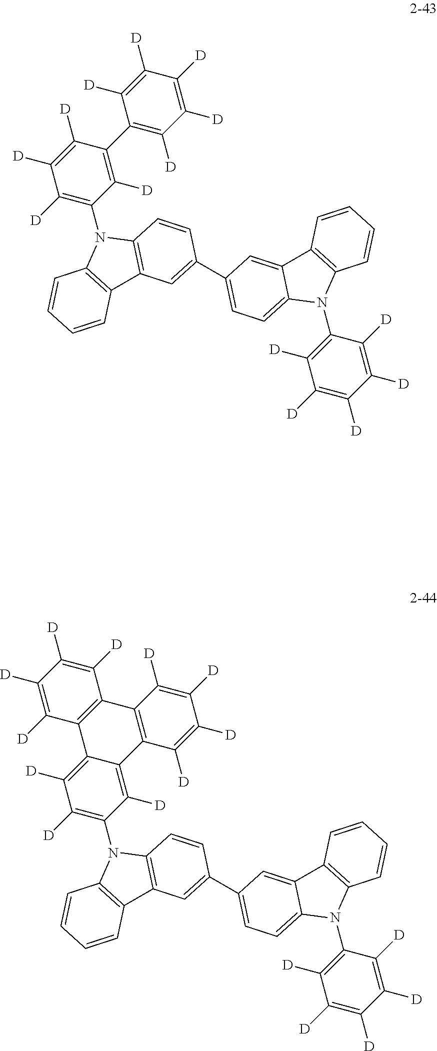 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c([2H])c([2H])c5[2H])ccc43)c2[2H])c([2H])c1[2H] ; [2H]c1c([2H])c([2H])c(-n2c3ccccc3c3cc(-c4ccc5c(c4)c4ccccc4n5-c4c([2H])c([2H])c5c6c([2H])c([2H])c([2H])c([2H])c6c6c([2H])c([2H])c([2H])c([2H])c6c5c4[2H])ccc32)c([2H])c1[2H]]

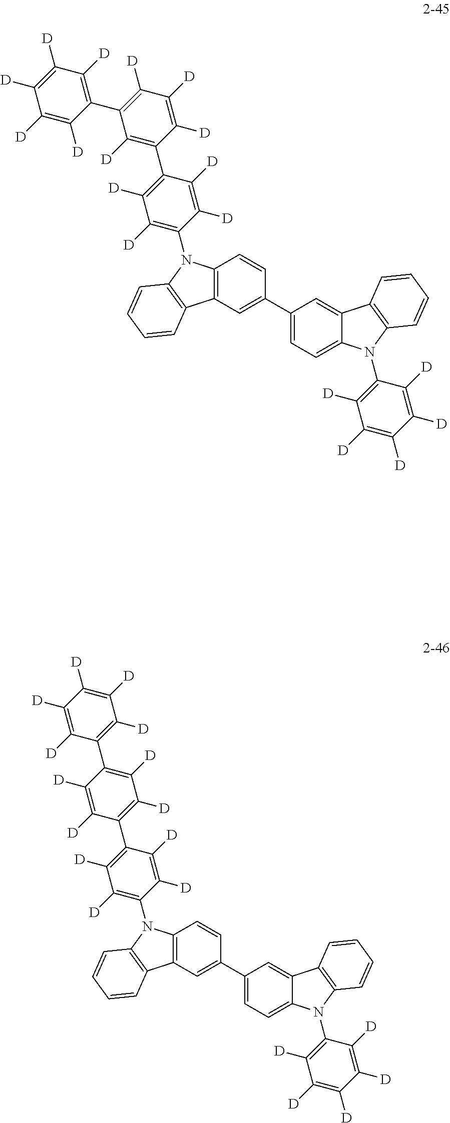 [SMILES: [2H]c1c([2H])c([2H])c(-c2c([2H])c([2H])c([2H])c(-c3c([2H])c([2H])c(-n4c5ccccc5c5cc(-c6ccc7c(c6)c6ccccc6n7-c6c([2H])c([2H])c([2H])c([2H])c6[2H])ccc54)c([2H])c3[2H])c2[2H])c([2H])c1[2H] ; [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c([2H])c([2H])c6[2H])ccc54)c([2H])c3[2H])c([2H])c2[2H])c([2H])c1[2H]]

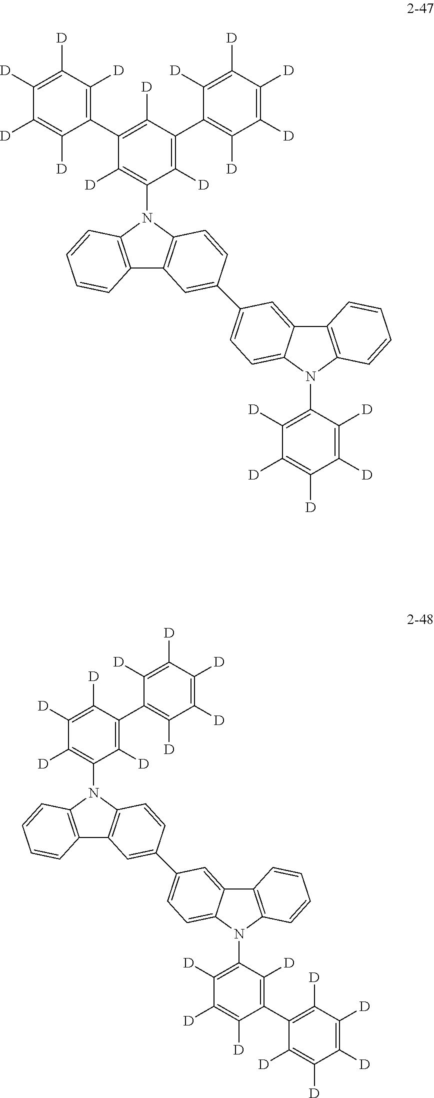 [SMILES: [2H]c1c([2H])c([2H])c(-c2c([2H])c(-c3c([2H])c([2H])c([2H])c([2H])c3[2H])c([2H])c(-n3c4ccccc4c4cc(-c5ccc6c(c5)c5ccccc5n6-c5c([2H])c([2H])c([2H])c([2H])c5[2H])ccc43)c2[2H])c([2H])c1[2H] ; [2H]c1c([2H])c([2H])c(-c2c([2H])c([2H])c([2H])c(-n3c4ccccc4c4cc(-c5ccc6c(c5)c5ccccc5n6-c5c([2H])c([2H])c([2H])c(-c6c([2H])c([2H])c([2H])c([2H])c6[2H])c5[2H])ccc43)c2[2H])c([2H])c1[2H]]

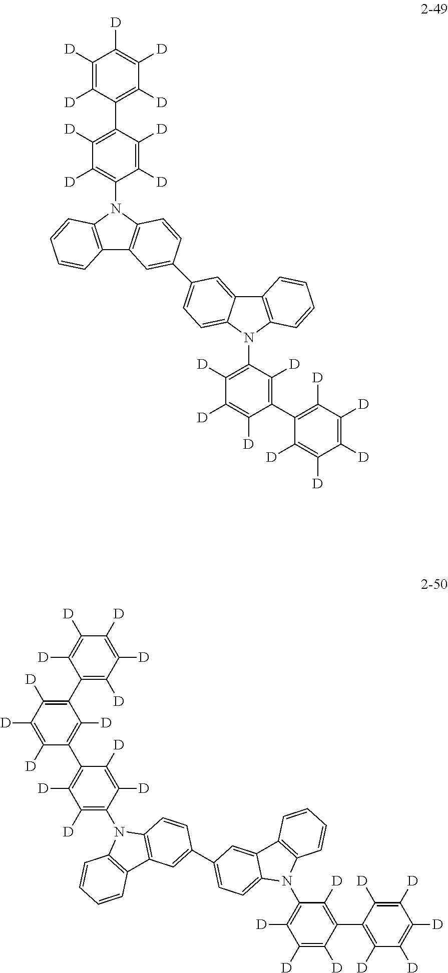 [SMILES: [2H]c1c([2H])c([2H])c(-c2c([2H])c([2H])c(-n3c4ccccc4c4cc(-c5ccc6c(c5)c5ccccc5n6-c5c([2H])c([2H])c([2H])c(-c6c([2H])c([2H])c([2H])c([2H])c6[2H])c5[2H])ccc43)c([2H])c2[2H])c([2H])c1[2H] ; [2H]c1c([2H])c([2H])c(-c2c([2H])c([2H])c([2H])c(-c3c([2H])c([2H])c(-n4c5ccccc5c5cc(-c6ccc7c(c6)c6ccccc6n7-c6c([2H])c([2H])c([2H])c(-c7c([2H])c([2H])c([2H])c([2H])c7[2H])c6[2H])ccc54)c([2H])c3[2H])c2[2H])c([2H])c1[2H]]

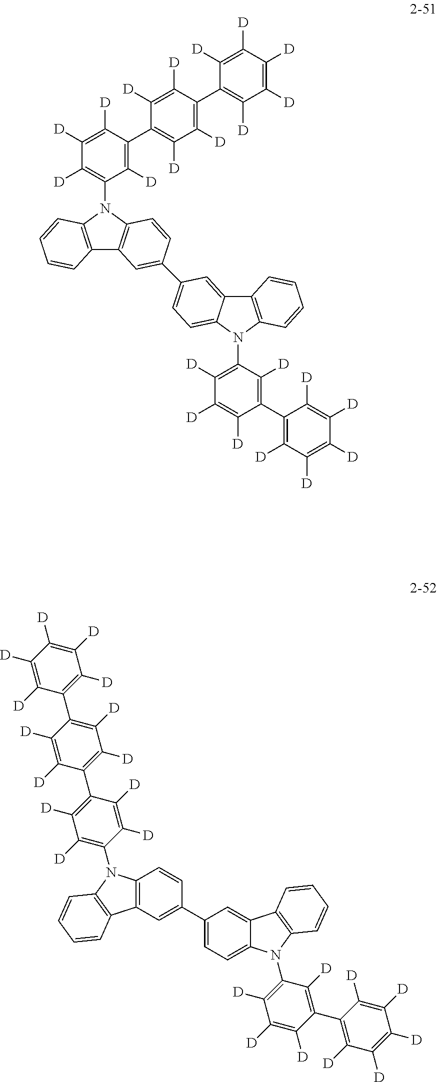 [SMILES: [2H]c1c([2H])c([2H])c(-c2c([2H])c([2H])c(-c3c([2H])c([2H])c([2H])c(-n4c5ccccc5c5cc(-c6ccc7c(c6)c6ccccc6n7-c6c([2H])c([2H])c([2H])c(-c7c([2H])c([2H])c([2H])c([2H])c7[2H])c6[2H])ccc54)c3[2H])c([2H])c2[2H])c([2H])c1[2H] ; [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c([2H])c(-c7c([2H])c([2H])c([2H])c([2H])c7[2H])c6[2H])ccc54)c([2H])c3[2H])c([2H])c2[2H])c([2H])c1[2H]]

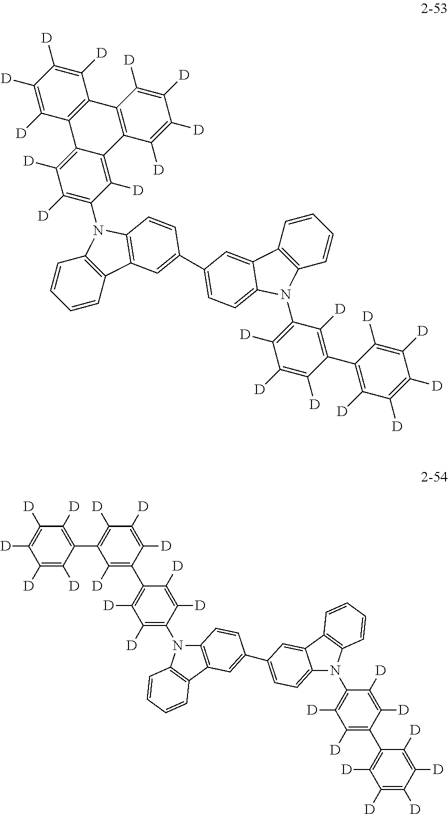 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c6c7c([2H])c([2H])c([2H])c([2H])c7c7c([2H])c([2H])c([2H])c([2H])c7c6c5[2H])ccc43)c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c(-c7c([2H])c([2H])c([2H])c([2H])c7[2H])c6[2H])c([2H])c5[2H])ccc43)c([2H])c2[2H])c([2H])c1[2H]]

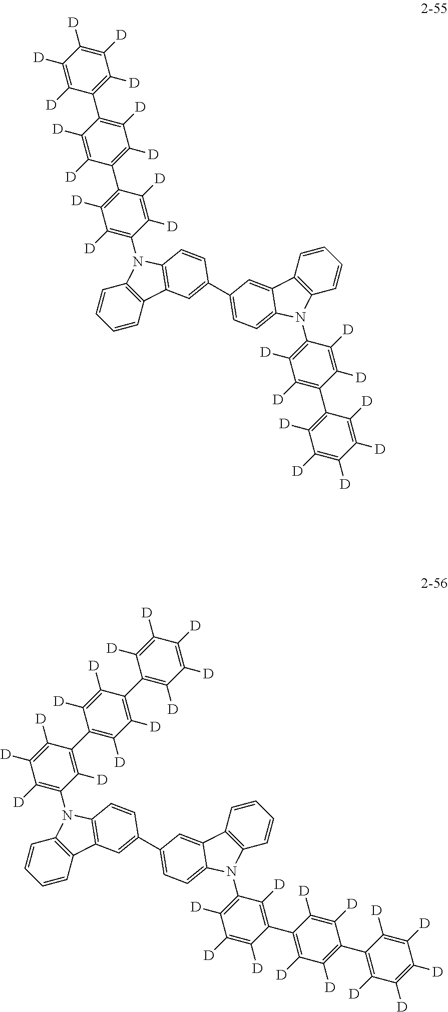 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c(-c7c([2H])c([2H])c([2H])c([2H])c7[2H])c([2H])c6[2H])ccc54)c([2H])c3[2H])c([2H])c2[2H])c([2H])c1[2H] ; [2H]c1c([2H])c([2H])c(-c2c([2H])c([2H])c(-c3c([2H])c([2H])c([2H])c(-n4c5ccccc5c5cc(-c6ccc7c(c6)c6ccccc6n7-c6c([2H])c([2H])c([2H])c(-c7c([2H])c([2H])c(-c8c([2H])c([2H])c([2H])c([2H])c8[2H])c([2H])c7[2H])c6[2H])ccc54)c3[2H])c([2H])c2[2H])c([2H])c1[2H]]

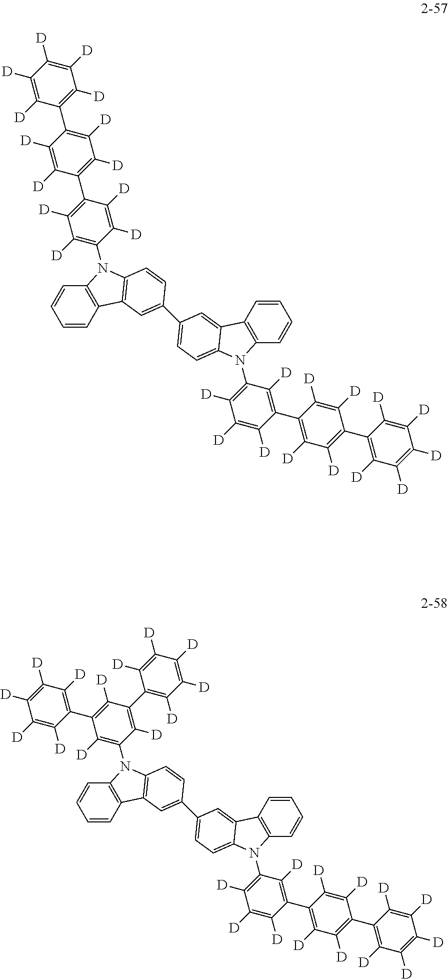

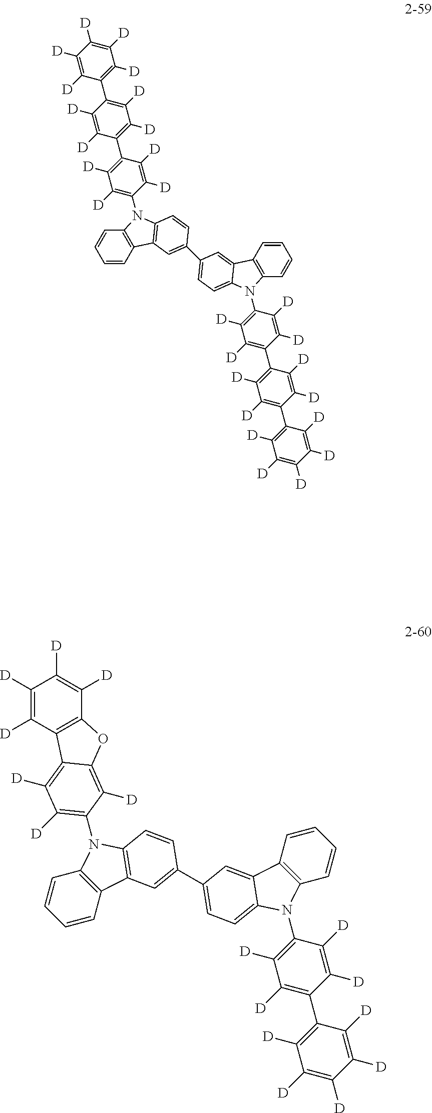 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c(-c7c([2H])c([2H])c(-c8c([2H])c([2H])c([2H])c([2H])c8[2H])c([2H])c7[2H])c([2H])c6[2H])ccc54)c([2H])c3[2H])c([2H])c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c6c(oc7c([2H])c([2H])c([2H])c([2H])c76)c5[2H])ccc43)c([2H])c2[2H])c([2H])c1[2H]]

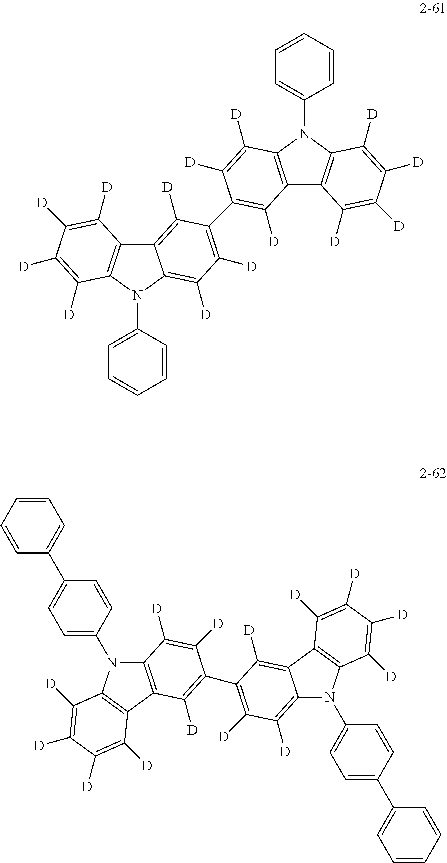 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3)c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccc(-c2ccccc2)cc1]

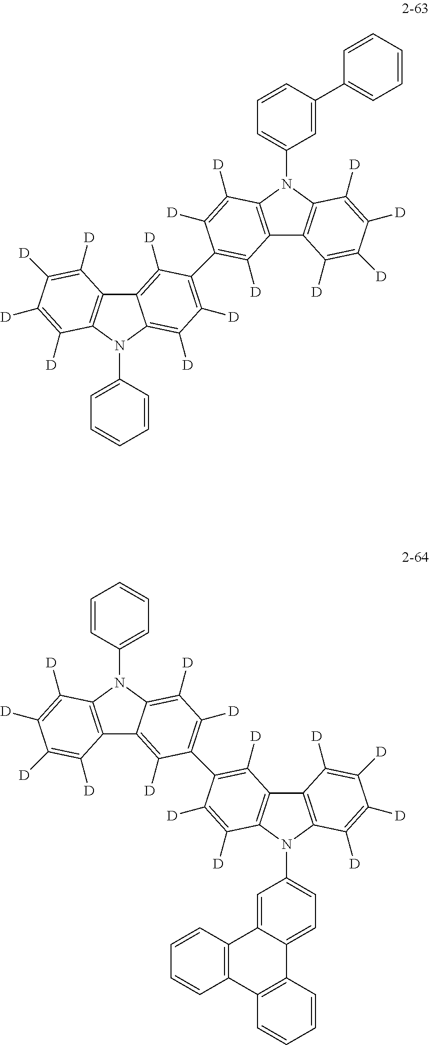 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c5ccccc5c5ccccc5c4c3)c([2H])c([2H])c1n2-c1ccccc1]

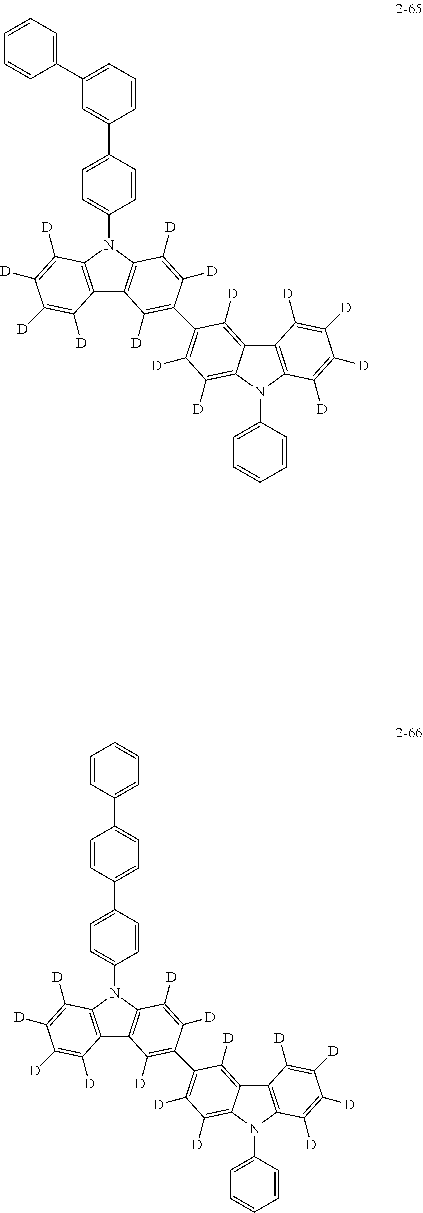 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4cccc(-c5ccccc5)c4)cc3)c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccc(-c5ccccc5)cc4)cc3)c([2H])c([2H])c1n2-c1ccccc1]

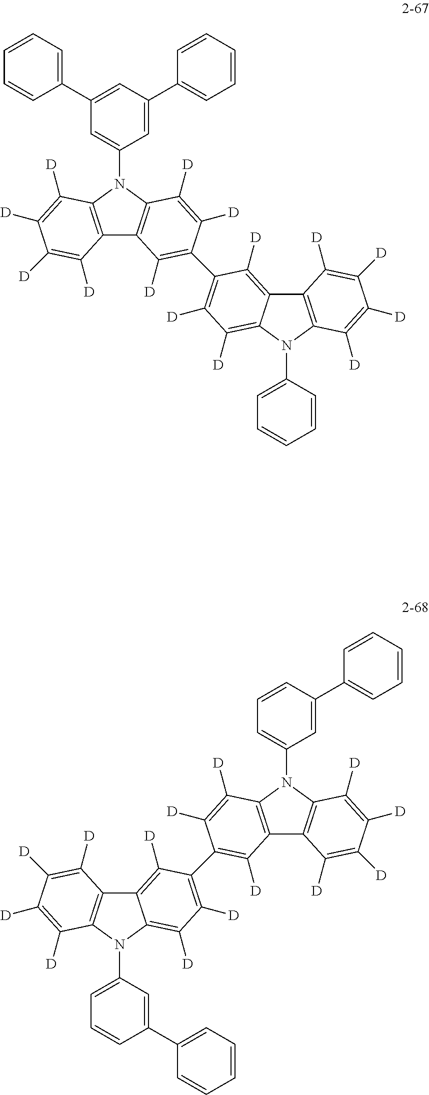 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cc(-c4ccccc4)cc(-c4ccccc4)c3)c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1cccc(-c2ccccc2)c1]

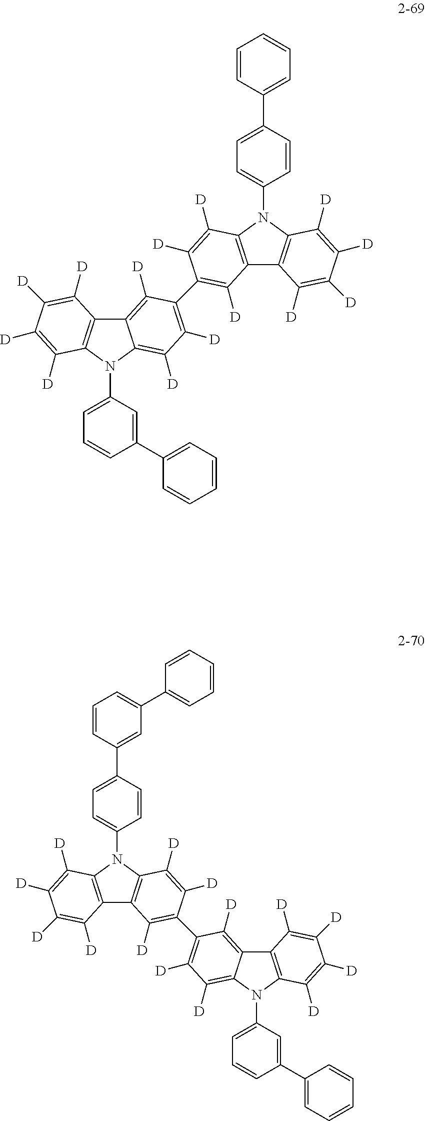 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1ccc(-c2ccccc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1ccc(-c2cccc(-c3ccccc3)c2)cc1]

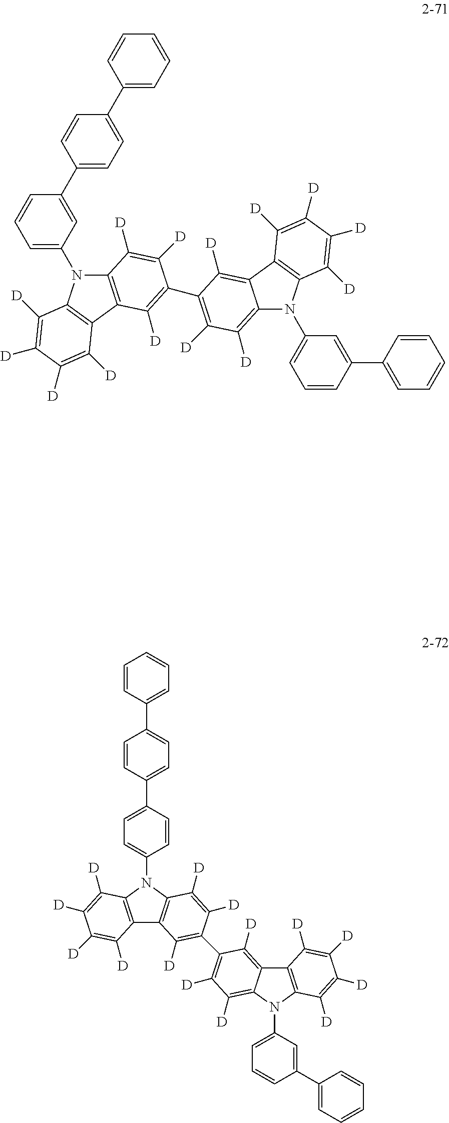 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccc(-c5ccccc5)cc4)c3)c([2H])c([2H])c1n2-c1cccc(-c2ccccc2)c1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1ccc(-c2ccc(-c3ccccc3)cc2)cc1]

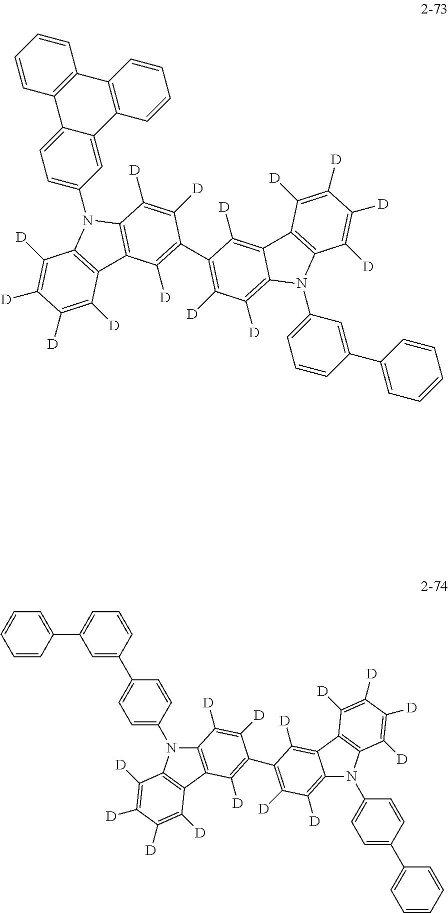 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c5ccccc5c5ccccc5c4c3)c([2H])c([2H])c1n2-c1cccc(-c2ccccc2)c1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4cccc(-c5ccccc5)c4)cc3)c([2H])c([2H])c1n2-c1ccc(-c2ccccc2)cc1]

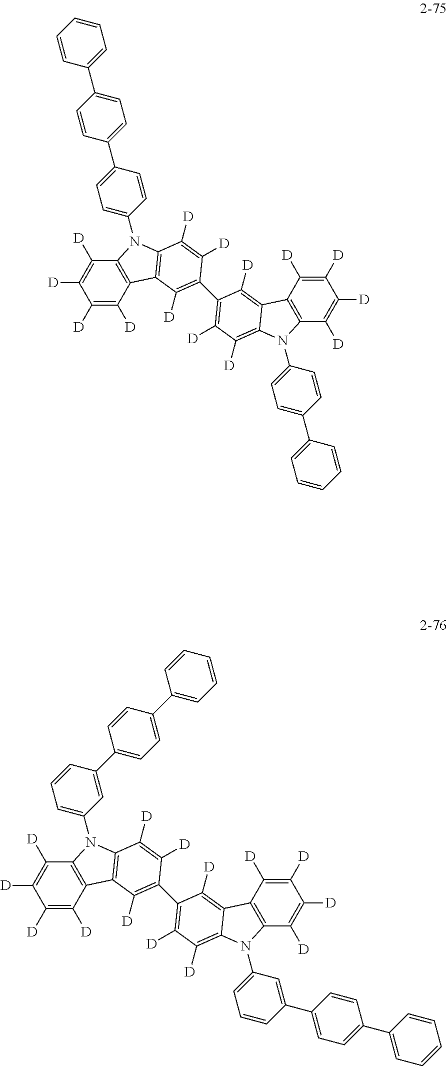 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccc(-c5ccccc5)cc4)cc3)c([2H])c([2H])c1n2-c1ccc(-c2ccccc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccc(-c5ccccc5)cc4)c3)c([2H])c([2H])c1n2-c1cccc(-c2ccc(-c3ccccc3)cc2)c1]

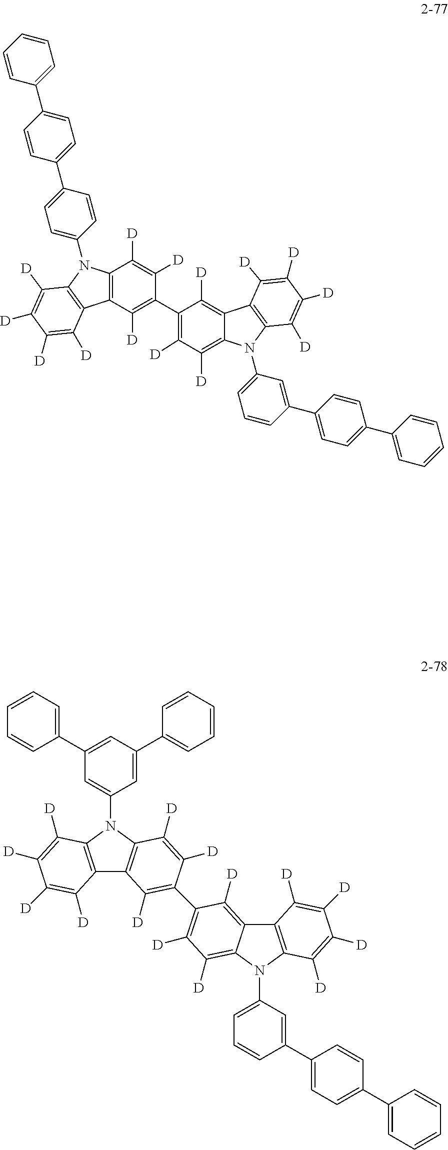 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccc(-c5ccccc5)cc4)c3)c([2H])c([2H])c1n2-c1ccc(-c2ccc(-c3ccccc3)cc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cc(-c4ccccc4)cc(-c4ccccc4)c3)c([2H])c([2H])c1n2-c1cccc(-c2ccc(-c3ccccc3)cc2)c1]

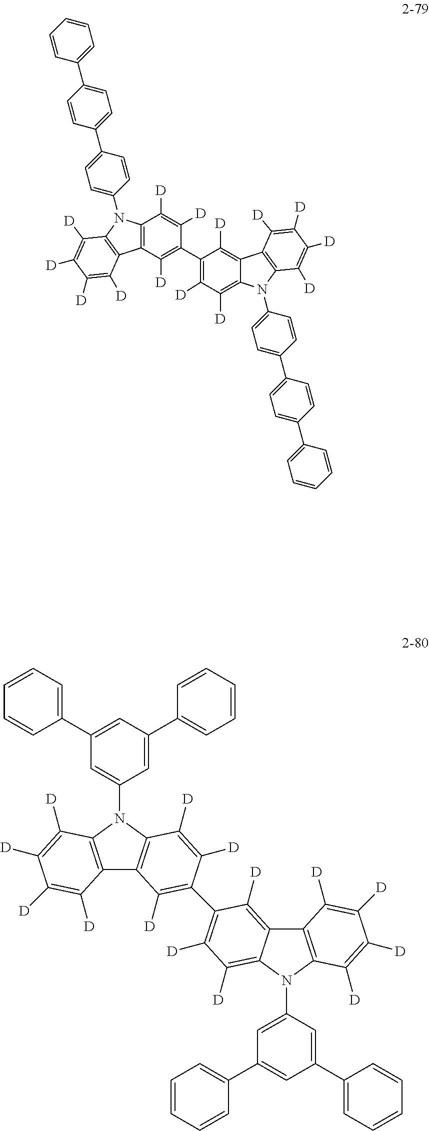 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccc(-c5ccccc5)cc4)cc3)c([2H])c([2H])c1n2-c1ccc(-c2ccc(-c3ccccc3)cc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cc(-c4ccccc4)cc(-c4ccccc4)c3)c([2H])c([2H])c1n2-c1cc(-c2ccccc2)cc(-c2ccccc2)c1]

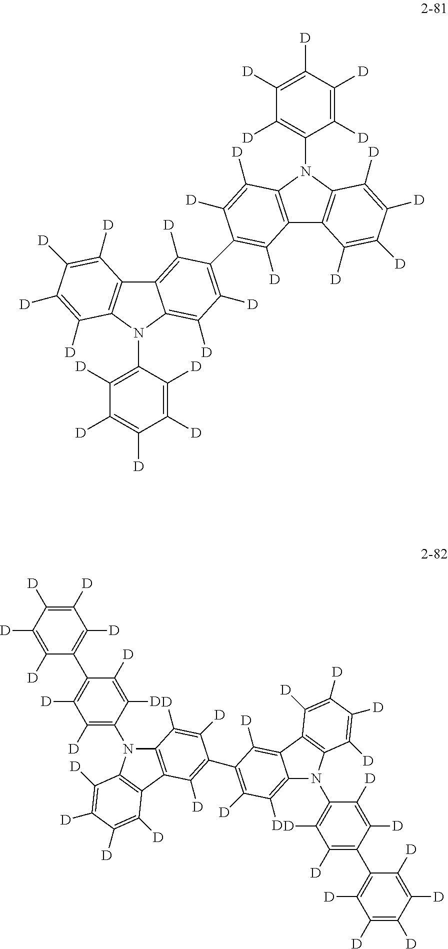 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c([2H])c4[2H])c([2H])c([2H])c32)c([2H])c1[2H] ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c(-c6c([2H])c([2H])c([2H])c([2H])c6[2H])c([2H])c5[2H])c([2H])c([2H])c43)c([2H])c2[2H])c([2H])c1[2H]]

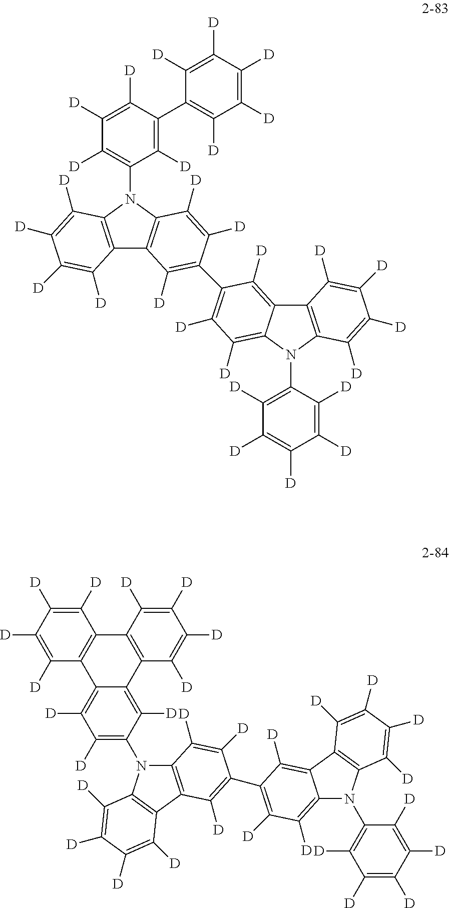 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c2[2H])c([2H])c1[2H] ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5c6c([2H])c([2H])c([2H])c([2H])c6c6c([2H])c([2H])c([2H])c([2H])c6c5c4[2H])c([2H])c([2H])c32)c([2H])c1[2H]]

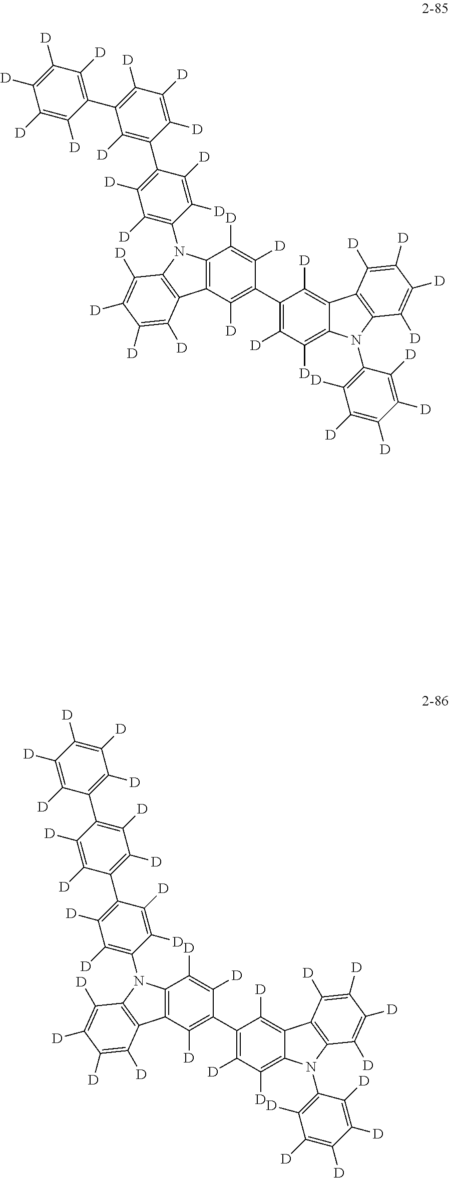

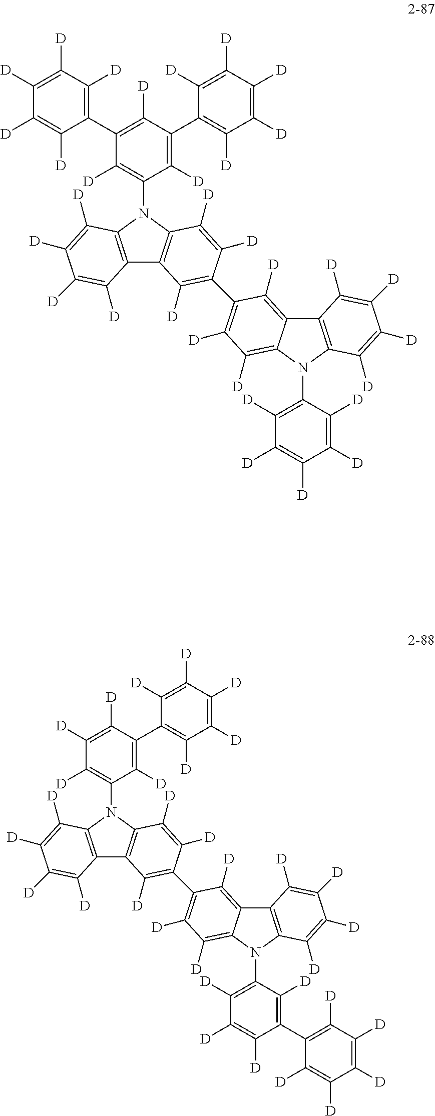

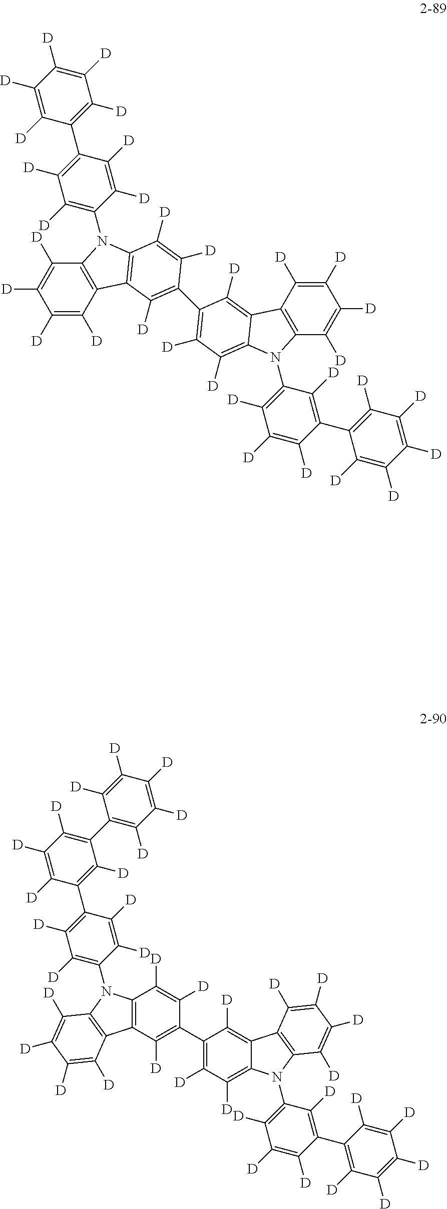

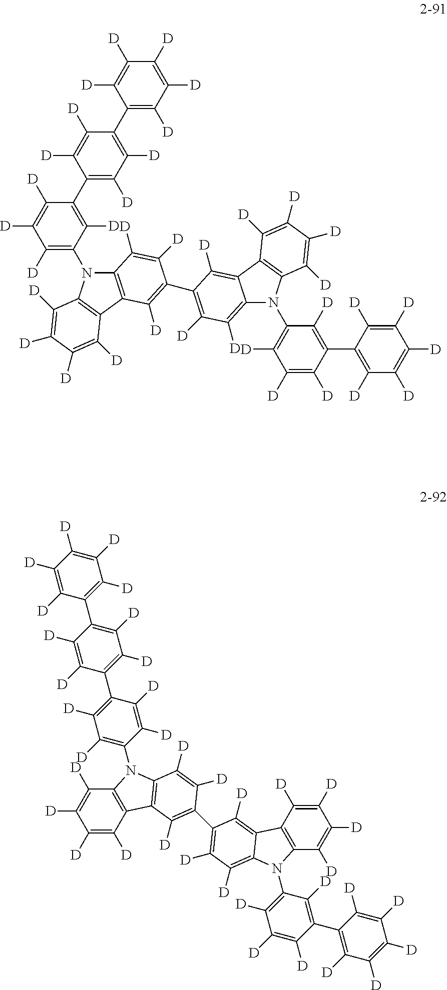

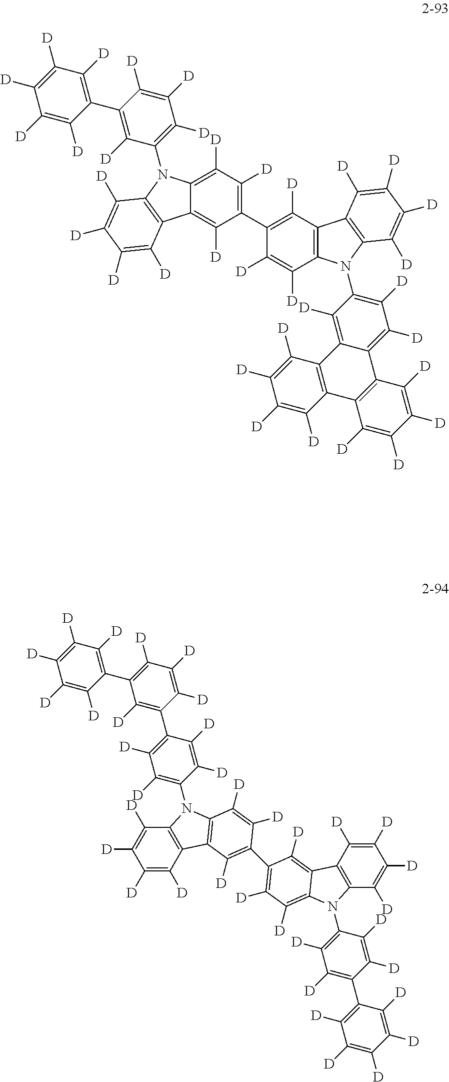

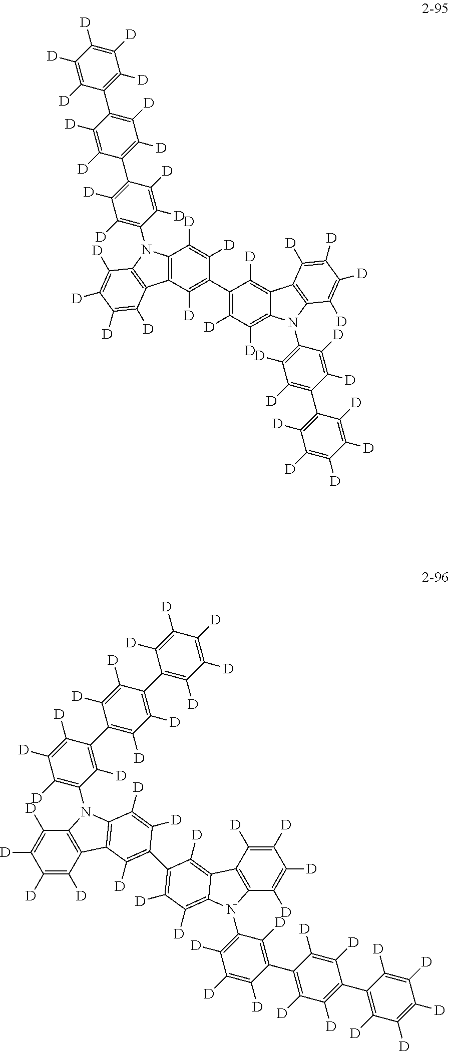

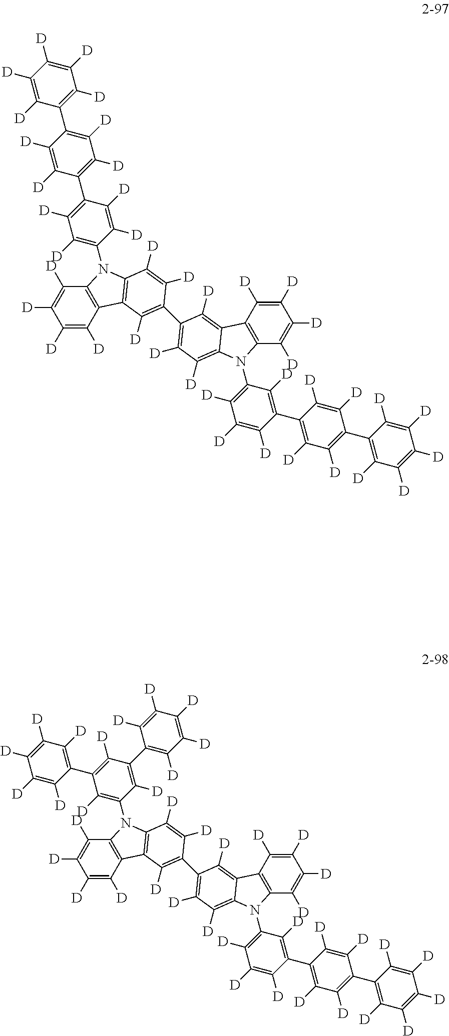

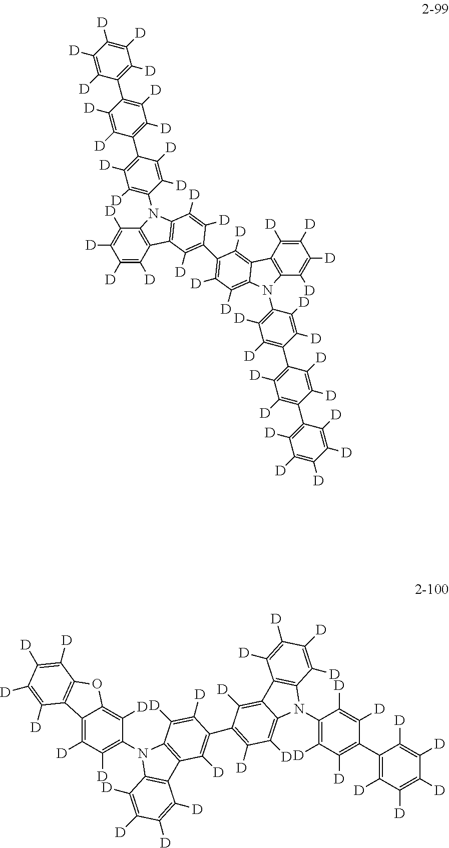

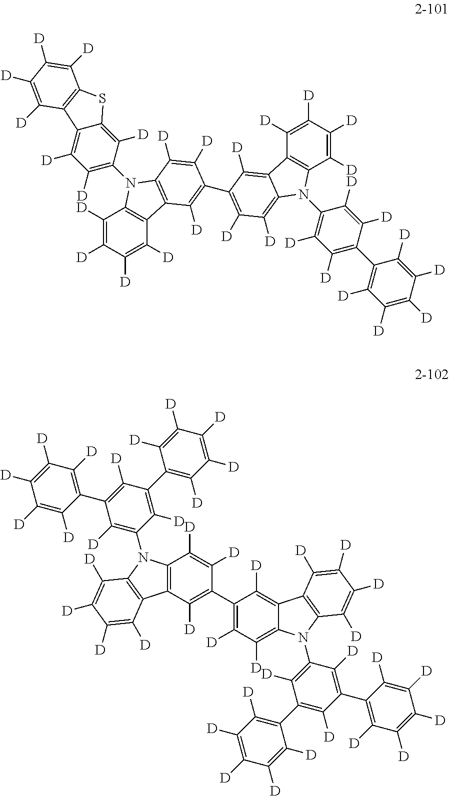

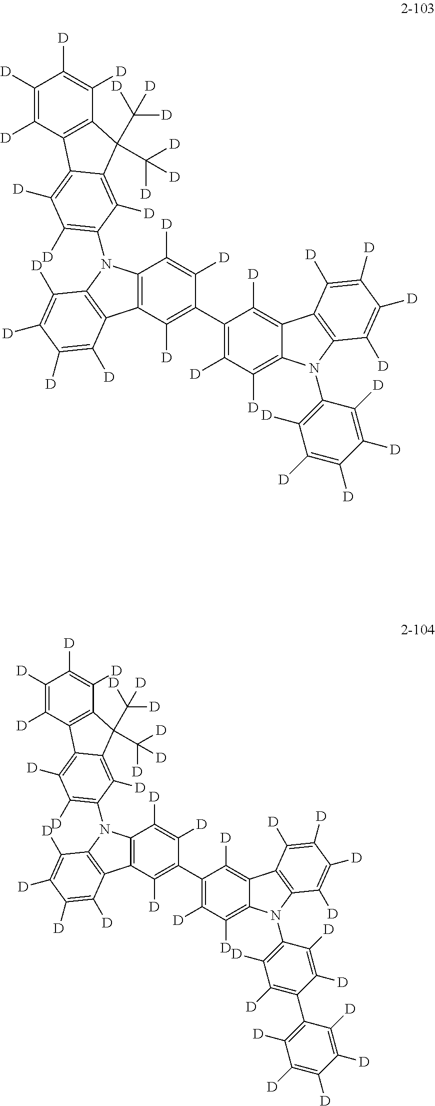

In addition, one embodiment of the present disclosure provides a composition for an organic material layer of an organic light emitting device, the composition comprising the heterocyclic compound represented by Chemical Formula 1 and the heterocyclic compound represented by Chemical Formula 10.

Specific descriptions on the heterocyclic compound represented by Chemical Formula 1 and the heterocyclic compound represented by Chemical Formula 10 are the same as the descriptions provided above.

In one embodiment of the present disclosure, the heterocyclic compound represented by Chemical Formula 1 and the heterocyclic compound represented by Chemical Formula 10 may have a weight ratio of 1:10 to 10:1, 1:8 to 8:1, 1:5 to 5:1 or 1:2 to 2:1 in the composition for an organic material layer of an organic light emitting device, however, the ratio is not limited thereto.

The composition for an organic material layer of an organic light emitting device may be used when forming an organic material of an organic light emitting device, and particularly, may be more preferably used when forming a host of a light emitting layer.

In one embodiment of the present disclosure, the organic material layer comprises the heterocyclic compound represented by Chemical Formula 1 and the heterocyclic compound represented by Chemical Formula 10, and a phosphorescent dopant may be used therewith.

As the phosphorescent dopant material, those known in the art may be used. For example, phosphorescent dopant materials represented by LL′MX′, LL′L″M, LMX′X″, L₂MX′ and L₃M may be used, however, the scope of the present disclosure is not limited to these examples.

M may be iridium, platinum, osmium or the like.

L is an anionic bidentate ligand coordinated to M by sp2 carbon and heteroatom, and X may function to trap electrons or holes. Nonlimiting examples of L may include 2-(1-naphthyl)benzoxazole, 2-phenylbenzoxazole, 2-phenylbenzothiazole, 7,8-benzoquinoline, phenylpyridine, benzothiophenylpyridine, 3-methoxy-2-phenylpyridine, thiophenylpyridine, tolylpyridine and the like. Nonlimiting examples of X′ and X″ may include acetylacetonate (acac), hexafluoroacetylacetonate, salicylidene, picolinate, 8-hydroxyquinolinate and the like.

Specific examples of the phosphorescent dopant are described below, however, the phosphorescent dopant is not limited to these examples.

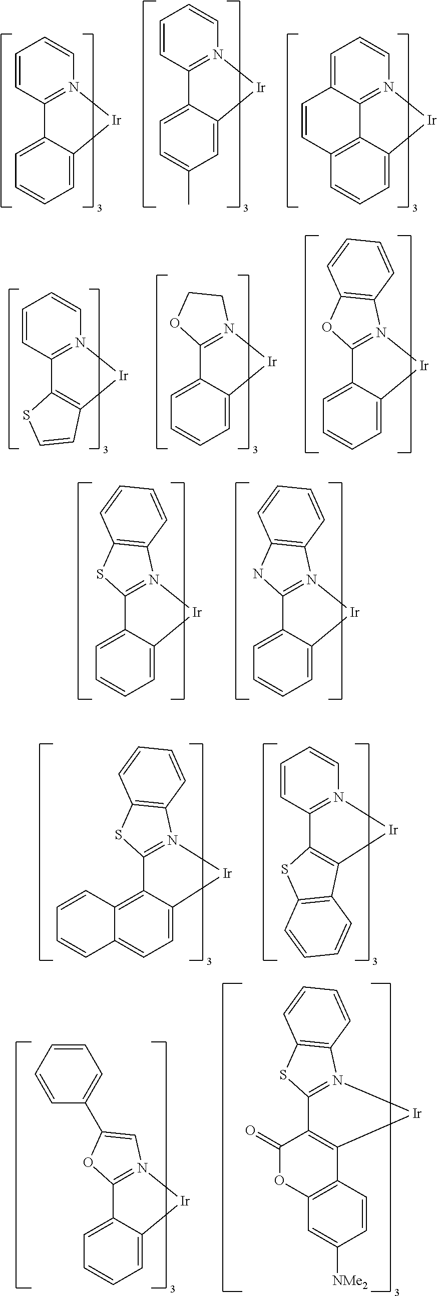

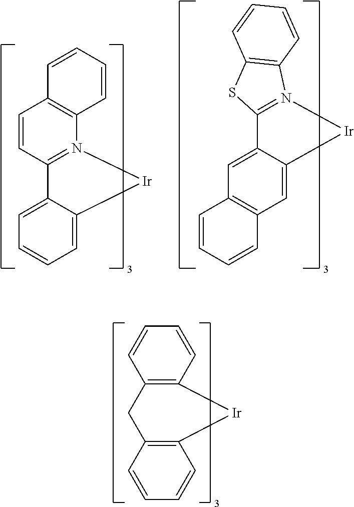

In one embodiment of the present disclosure, the organic material layer comprises the heterocyclic compound represented by Chemical Formula 1 and the heterocyclic compound represented by Chemical Formula 10, and an iridium-based dopant may be used therewith.

In one embodiment of the present disclosure, as the iridium-based dopant, (piq)₂(Ir) (acac) may be used as a red phosphorescent dopant, or Ir(ppy)₃ may be used as a green phosphorescent dopant and (piq)₂(Ir) (acac) may be used as a red phosphorescent dopant.

In one embodiment of the present disclosure, a content of the dopant may be from 1% to 15%, preferably from 2% to 10% and more preferably from 3% to 7% based on the total weight of the light emitting layer.

In the organic light emitting device according to one embodiment of the present disclosure, the organic material layer includes an electron injection layer or an electron transport layer, and the electron injection layer or the electron transport layer may include the heterocyclic compound.

In the organic light emitting device according to another embodiment of the present disclosure, the organic material layer includes an electron blocking layer or a hole blocking layer, and the electron blocking layer or the hole blocking layer may include the heterocyclic compound.

In the organic light emitting device according to another embodiment, the organic material layer includes an electron transport layer, a light emitting layer or a hole blocking layer, and the electron transport layer, the light emitting layer or the hole blocking layer may include the heterocyclic compound.

In the organic light emitting device according to another embodiment, the organic material layer includes a light emitting layer, and the light emitting layer may include the heterocyclic compound.

In the organic light emitting device according to another embodiment, the organic material layer includes a light emitting layer, the light emitting layer includes a host material, and the host material may include the heterocyclic compound represented by Chemical Formula 1.

In the organic light emitting device according to another embodiment, the light emitting layer may include two or more host materials, and at least one of the host materials may include the heterocyclic compound represented by Chemical Formula 1, and the other one may include the heterocyclic compound represented by Chemical Formula 6.

In the organic light emitting device according to another embodiment, two or more host materials may be pre-mixed and used in the light emitting layer, and at least one of the two or more host materials may include the heterocyclic compound represented by Chemical Formula 1, and the other one may include the heterocyclic compound represented by Chemical Formula 6.

The pre-mixing means first mixing the two or more host materials of the light emitting layer in one source of supply before depositing on the organic material layer.

The organic light emitting device according to one embodiment of the present disclosure may further include one, two or more layers selected from the group consisting of a light emitting layer, a hole injection layer, a hole transport layer, an electron injection layer, an electron transport layer, an electron blocking layer and a hole blocking layer.

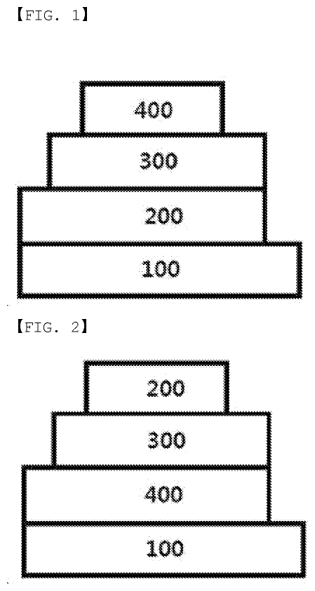

FIG. 1 to FIG. 3 illustrate a lamination order of electrodes and organic material layers of the organic light emitting device according to one embodiment of the present disclosure. However, the scope of the present application is not limited to these diagrams, and structures of organic light emitting devices known in the art may also be used in the present application.

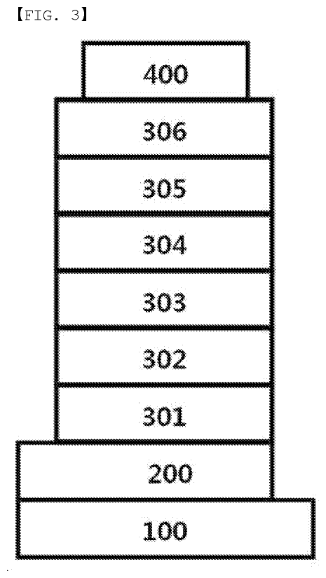

FIG. 1 illustrates the organic light emitting device in which a positive electrode (200), an organic material layer (300) and a negative electrode (400) are consecutively laminated on a substrate (100). However, the structure is not limited to such a structure, and as illustrated in FIG. 2, an organic light emitting device in which a negative electrode, an organic material layer and a positive electrode are consecutively laminated on a substrate may also be obtained.

FIG. 3 illustrates a case of the organic material layer being a multilayer. The organic light emitting device according to FIG. 3 includes a hole injection layer (301), a hole transport layer (302), a light emitting layer (303), a hole blocking layer (304), an electron transport layer (305) and an electron injection layer (306). However, the scope of the present application is not limited to such a lamination structure, and as necessary, the layers other than the light emitting layer may not be included, and other necessary functional layers may be further added.

One embodiment of the present disclosure provides a method for manufacturing an organic light emitting device, the method comprising the steps of:
preparing a substrate;
forming a first electrode on the substrate;
forming one or more organic material layers on the first electrode; and
forming a second electrode on the one or more organic material layers,
wherein the steps of forming the one or more organic material layers includes a steps of forming the one or more organic material layers using the composition for an organic material layer according to one embodiment of the present disclosure.

In one embodiment of the present disclosure, the forming of organic material layers may be forming using a thermal vacuum deposition method after pre-mixing the heterocyclic compound represented by Chemical Formula 1 and the heterocyclic compound represented by Chemical Formula 10.

The pre-mixing means first mixing the heterocyclic compound represented by Chemical Formula 1 and the heterocyclic compound represented by Chemical Formula 10 in one source of supply before depositing on the organic material layer.

The pre-mixed material may be referred to as the composition for an organic material layer according to one embodiment of the present application.

The organic material layer including the heterocyclic compound represented by Chemical Formula 1 may further include other materials as necessary.

The organic material layer including the heterocyclic compound represented by Chemical Formula 1 and the heterocyclic compound represented by Chemical Formula 10 at the same time may further include other materials as necessary.

In the organic light emitting device according to one embodiment of the present disclosure, materials other than the heterocyclic compound represented by Chemical Formula 1 or the heterocyclic compound represented by Chemical Formula 10 are illustrated below, however, these are for illustrative purposes only and not for limiting the scope of the present application, and these materials may be replaced by materials known in the art.

As the positive electrode material, materials having relatively large work function may be used, and transparent conductive oxides, metals, conductive polymers or the like may be used. Specific examples of the positive electrode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the negative electrode material, materials having relatively small work function may be used, and metals, metal oxides, conductive polymers or the like may be used. Specific examples of the negative electrode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection layer material, known hole injection layer materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tris[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], conductive polymers having solubility such as polyaniline/dodecylbenzenesulfonic acid or poly(3,4-ethylenedioxythiophene)/poly(4-styrenesulfonate), polyaniline/camphor sulfonic acid or polyaniline/poly(4-styrenesulfonate), and the like, may be used.

As the hole transport layer material, pyrazoline derivatives, arylamine-based derivatives, stilbene derivatives, triphenyldiamine derivatives and the like may be used, and low molecular or high molecular materials may also be used.

As the electron transport layer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials.

As examples of the electron injection layer material, LiF is typically used in the art, however, the present application is not limited thereto.

As the light emitting layer material, red, green or blue light emitting materials may be used, and as necessary, two or more light emitting materials may be mixed and used. Herein, the two or more light emitting materials may be deposited as individual sources of supply or pre-mixed and deposited as one source of supply when used. In addition, fluorescent materials may also be used as the light emitting layer material, however, phosphorescent materials may also be used. As the light emitting layer material, materials emitting light by binding holes and electrons injected from a positive electrode and a negative electrode, respectively, may be used alone, however, materials having a host material and a dopant material involving together in light emission may also be used.

When mixing hosts of the light emitting layer material, same series hosts may be mixed, or different series hosts may be mixed. For example, any two or more types of materials among n-type host materials or p-type host materials may be selected and used as a host material of a light emitting layer.

The organic light emitting device according to one embodiment of the present disclosure may be a top-emission type, a bottom-emission type or a dual-emission type depending on the materials used.

The heterocyclic compound according to one embodiment of the present disclosure may also be used in an organic electronic device including an organic solar cell, an organic photo conductor, an organic transistor and the like under a similar principle used in the organic light emitting device.

Hereinafter, preferred examples are provided to illuminate the present disclosure, however, the following examples are provided to more readily understand the present disclosure, and the present disclosure is not limited thereto.

### PREPARATION EXAMPLE

### Preparation Example 1. Preparation of Compound 1-1

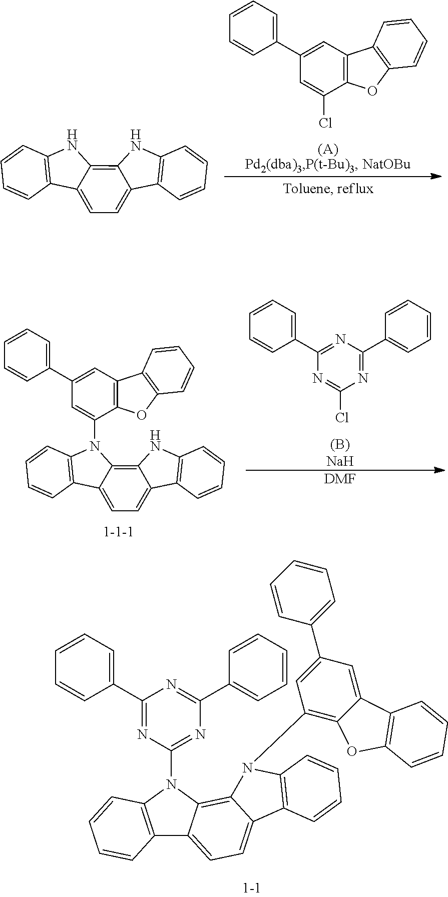

### Preparation Example 1-1. Preparation of Compound 1-1-1

4-Chloro-2-phenyldibenzo[b,d]furan (A) (25.1 g, 90 mM), 11,12-dihydroindolo[2,3-a]carbazole (25.6 g, 100 mM), tris(dibenzylideneacetone)dipalladium (Pd₂(dba)₃) (4.57 g, 5 mM), tri-tert-butylphosphine (P(t-Bu)₃) (2.02 g, 10 mM) and sodium tert-butoxide (NatOBu) (19.22 g, 200 mM) were dissolved in toluene (250 mL), and refluxed for 24 hours.

After the reaction was completed, the result was extracted by introducing distilled water and dichloromethane (DCM) thereto at room temperature, and after drying the organic layer with anhydrous magnesium sulfate (MgSO₄), the solvent was removed using a rotary evaporator. The reaction material was purified by column chromatography (dichloromethane:hexane=1:2), and recrystallized with methanol to obtain target Compound 1-1-1 (31.4 g).

### Preparation Example 1-2. Preparation of Compound 1-1

After dissolving Compound 1-1-1 (31 g, 62.1 mM) in dimethylformamide (DMF) (300 mL), sodium hydride (NaH) (2.98 g, 124.2 mM) was slowly introduced thereto. After 1 hour, 2-chloro-4,6-diphenyl-1,3,5-triazine (B) (21.6 g, 80.7 mM) was introduced thereto, and the mixture was reacted for 6 hours.

After the reaction was completed, the result was extracted by introducing distilled water and dichloromethane (DCM) thereto at room temperature, and after drying the organic layer with anhydrous magnesium sulfate (MgSO₄), the solvent was removed using a rotary evaporator. The reaction material was purified by column chromatography (dichloromethane:hexane=1:2), and recrystallized with methanol to obtain target Compound 1-1 (39.5 g, yield 87%).

Target compounds were synthesized as in the following Table 1 in the same manner as in Preparation Example 1 except that Compound A of the following Table 1 was used instead of 4-chloro-2-phenyldibenzo[b,d]furan (A), and Compound B of the following Table 1 was used instead of 2-chloro-4,6-diphenyl-1,3,5-triazine (B).

TABLE 1

| Com- | | |
|---|---|---|
| pound | | |
| No. | Compound A | Compound B |
| 1-2 | 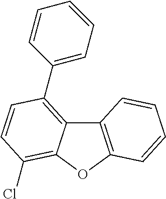 [SMILES: Clc1ccc(-c2ccccc2)c2c1oc1ccccc12] | 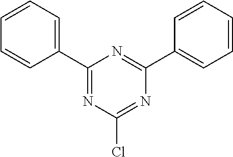 [SMILES: Clc1nc(-c2ccccc2)nc(-c2ccccc2)n1] |
| 1-3 | 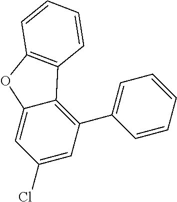 [SMILES: Clc1cc(-c2ccccc2)c2c(c1)oc1ccccc12] | 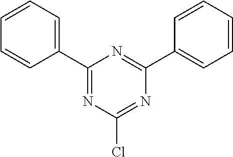 [SMILES: Clc1nc(-c2ccccc2)nc(-c2ccccc2)n1] |
| 1-4 | 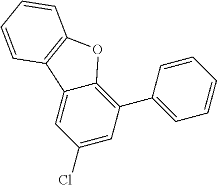 [SMILES: Clc1cc(-c2ccccc2)c2oc3ccccc3c2c1] | 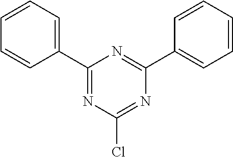 [SMILES: Clc1nc(-c2ccccc2)nc(-c2ccccc2)n1] |
| 1-5 | 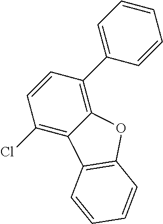 [SMILES: Clc1ccc(-c2ccccc2)c2oc3ccccc3c12] | 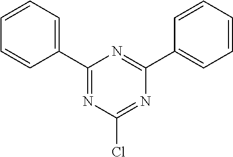 [SMILES: Clc1nc(-c2ccccc2)nc(-c2ccccc2)n1] |
| 1-6 | 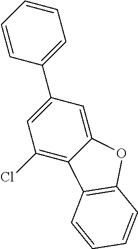 [SMILES: Clc1cc(-c2ccccc2)cc2oc3ccccc3c12] | 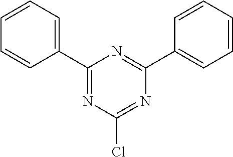 [SMILES: Clc1nc(-c2ccccc2)nc(-c2ccccc2)n1] |
| 1-11 | 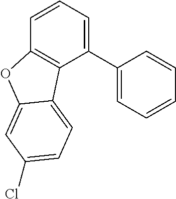 [SMILES: Clc1ccc2c(c1)oc1cccc(-c3ccccc3)c12] | 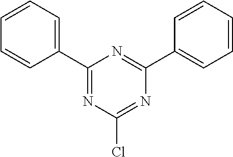 [SMILES: Clc1nc(-c2ccccc2)nc(-c2ccccc2)n1] |
| 1-12 | 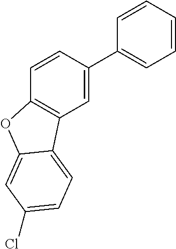 [SMILES: Clc1ccc2c(c1)oc1ccc(-c3ccccc3)cc12] | 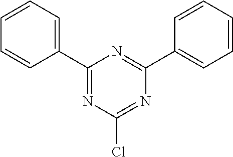 [SMILES: Clc1nc(-c2ccccc2)nc(-c2ccccc2)n1] |
| 1-13 | 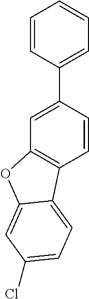 [SMILES: Clc1ccc2c(c1)oc1cc(-c3ccccc3)ccc12] | 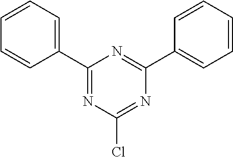 [SMILES: Clc1nc(-c2ccccc2)nc(-c2ccccc2)n1] |
| 1-14 | 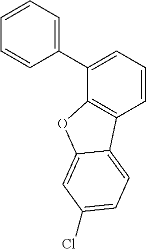 [SMILES: Clc1ccc2c(c1)oc1c(-c3ccccc3)cccc12] | 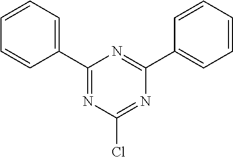 [SMILES: Clc1nc(-c2ccccc2)nc(-c2ccccc2)n1] |
| 1-15 | 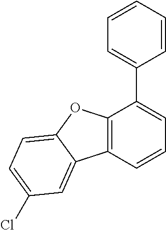 [SMILES: Clc1ccc2oc3c(-c4ccccc4)cccc3c2c1] | 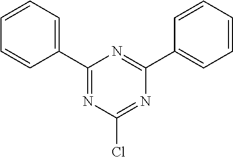 [SMILES: Clc1nc(-c2ccccc2)nc(-c2ccccc2)n1] |
| 1-17 | 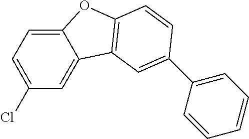 [SMILES: Clc1ccc2oc3ccc(-c4ccccc4)cc3c2c1] | 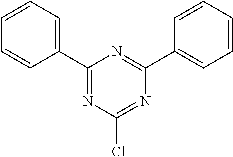 [SMILES: Clc1nc(-c2ccccc2)nc(-c2ccccc2)n1] |
| 1-21 | 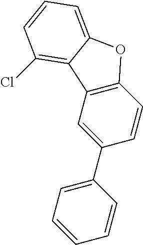 [SMILES: Clc1cccc2oc3ccc(-c4ccccc4)cc3c12] | 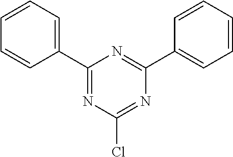 [SMILES: Clc1nc(-c2ccccc2)nc(-c2ccccc2)n1] |
| 1-22 | 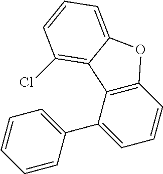 [SMILES: Clc1cccc2oc3cccc(-c4ccccc4)c3c12] | 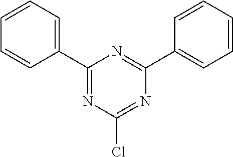 [SMILES: Clc1nc(-c2ccccc2)nc(-c2ccccc2)n1] |
| 1-23 | 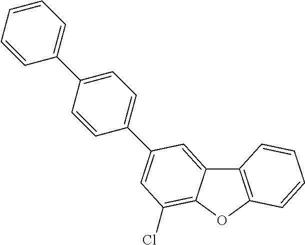 [SMILES: Clc1cc(-c2ccc(-c3ccccc3)cc2)cc2c1oc1ccccc12] | 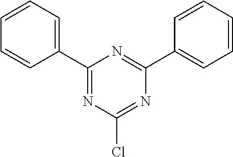 [SMILES: Clc1nc(-c2ccccc2)nc(-c2ccccc2)n1] |
| 1-24 | 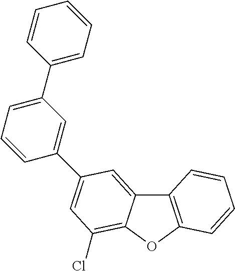 [SMILES: Clc1cc(-c2cccc(-c3ccccc3)c2)cc2c1oc1ccccc12] | 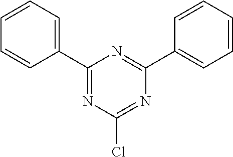 [SMILES: Clc1nc(-c2ccccc2)nc(-c2ccccc2)n1] |
| 1-27 | 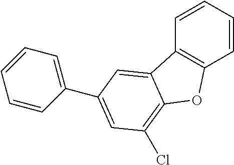 [SMILES: Clc1cc(-c2ccccc2)cc2c1oc1ccccc12] | 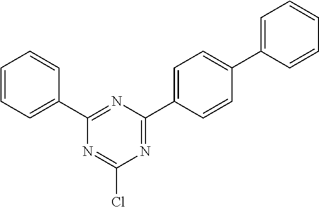 [SMILES: Clc1nc(-c2ccccc2)nc(-c2ccc(-c3ccccc3)cc2)n1] |
| 1-33 | 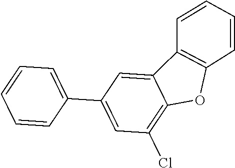 [SMILES: Clc1cc(-c2ccccc2)cc2c1oc1ccccc12] | 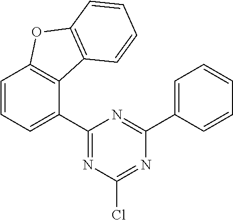 [SMILES: Clc1nc(-c2ccccc2)nc(-c2cccc3oc4ccccc4c23)n1] |
| 1-34 | 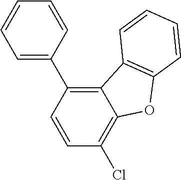 [SMILES: Clc1ccc(-c2ccccc2)c2c1oc1ccccc12] | 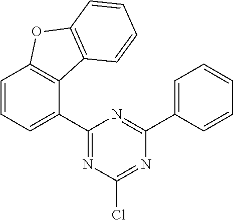 [SMILES: Clc1nc(-c2ccccc2)nc(-c2cccc3oc4ccccc4c23)n1] |
| 1-35 | 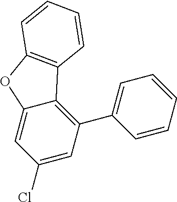 [SMILES: Clc1cc(-c2ccccc2)c2c(c1)oc1ccccc12] | 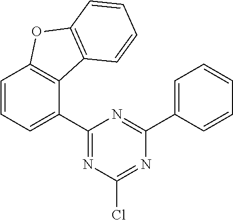 [SMILES: Clc1nc(-c2ccccc2)nc(-c2cccc3oc4ccccc4c23)n1] |
| 1-36 | 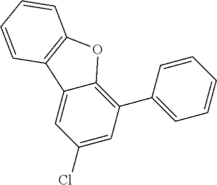 [SMILES: Clc1cc(-c2ccccc2)c2oc3ccccc3c2c1] | 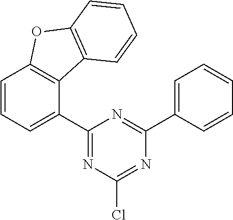 [SMILES: Clc1nc(-c2ccccc2)nc(-c2cccc3oc4ccccc4c23)n1] |
| 1-37 | 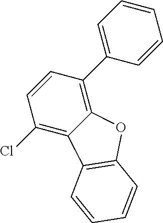 [SMILES: Clc1ccc(-c2ccccc2)c2oc3ccccc3c12] | 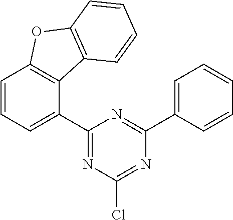 [SMILES: Clc1nc(-c2ccccc2)nc(-c2cccc3oc4ccccc4c23)n1] |
| 1-38 | 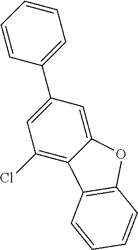 [SMILES: Clc1cc(-c2ccccc2)cc2oc3ccccc3c12] | 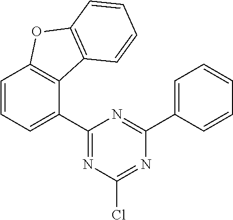 [SMILES: Clc1nc(-c2ccccc2)nc(-c2cccc3oc4ccccc4c23)n1] |
| 1-43 | 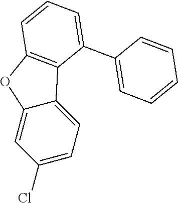 [SMILES: Clc1ccc2c(c1)oc1cccc(-c3ccccc3)c12] | 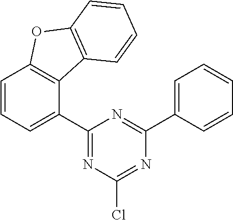 [SMILES: Clc1nc(-c2ccccc2)nc(-c2cccc3oc4ccccc4c23)n1] |
| 1-44 | 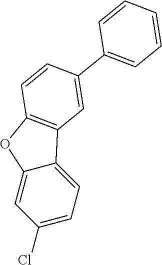 [SMILES: Clc1ccc2c(c1)oc1ccc(-c3ccccc3)cc12] | 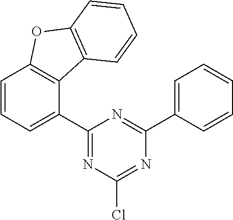 [SMILES: Clc1nc(-c2ccccc2)nc(-c2cccc3oc4ccccc4c23)n1] |
| 1-47 | 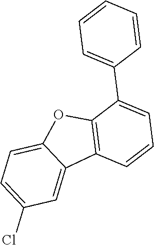 [SMILES: Clc1ccc2oc3c(-c4ccccc4)cccc3c2c1] | 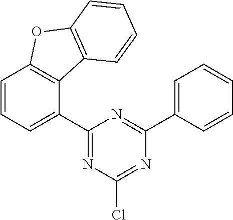 [SMILES: Clc1nc(-c2ccccc2)nc(-c2cccc3oc4ccccc4c23)n1] |
| 1-48 | 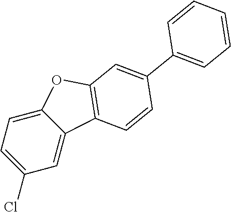 [SMILES: Clc1ccc2oc3cc(-c4ccccc4)ccc3c2c1] | 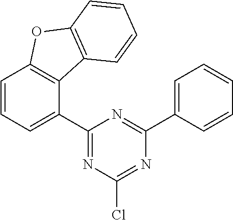 [SMILES: Clc1nc(-c2ccccc2)nc(-c2cccc3oc4ccccc4c23)n1] |
| 1-51 | 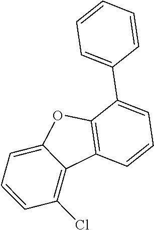 [SMILES: Clc1cccc2oc3c(-c4ccccc4)cccc3c12] | 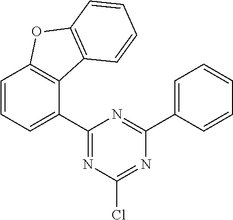 [SMILES: Clc1nc(-c2ccccc2)nc(-c2cccc3oc4ccccc4c23)n1] |
| 1-52 | 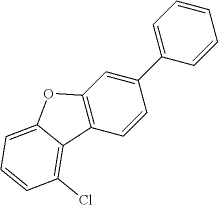 [SMILES: Clc1cccc2oc3cc(-c4ccccc4)ccc3c12] | 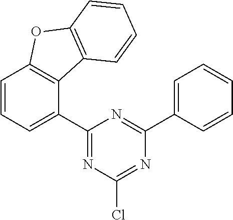 [SMILES: Clc1nc(-c2ccccc2)nc(-c2cccc3oc4ccccc4c23)n1] |
| 1-55 | 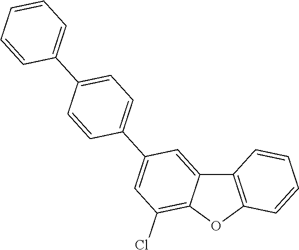 [SMILES: Clc1cc(-c2ccc(-c3ccccc3)cc2)cc2c1oc1ccccc12] | 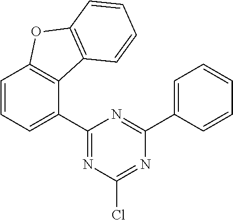 [SMILES: Clc1nc(-c2ccccc2)nc(-c2cccc3oc4ccccc4c23)n1] |
| 1-56 | 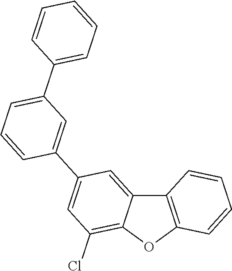 [SMILES: Clc1cc(-c2cccc(-c3ccccc3)c2)cc2c1oc1ccccc12] | 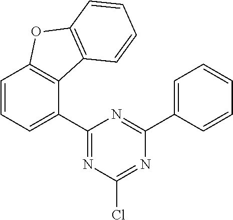 [SMILES: Clc1nc(-c2ccccc2)nc(-c2cccc3oc4ccccc4c23)n1] |
| 1-61 | 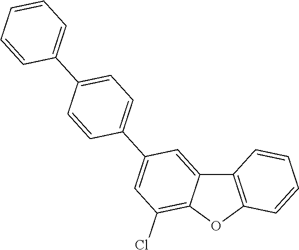 [SMILES: Clc1cc(-c2ccc(-c3ccccc3)cc2)cc2c1oc1ccccc12] | 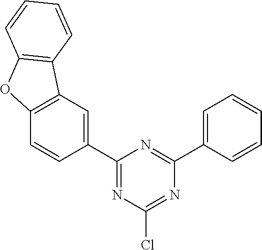 [SMILES: Clc1nc(-c2ccccc2)nc(-c2ccc3oc4ccccc4c3c2)n1] |
| 1-62 | 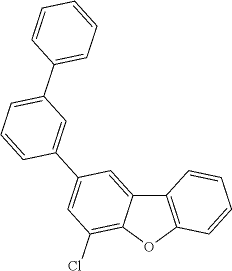 [SMILES: Clc1cc(-c2cccc(-c3ccccc3)c2)cc2c1oc1ccccc12] | 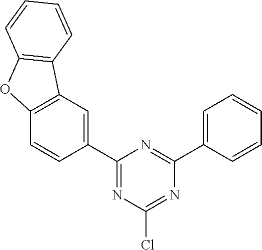 [SMILES: Clc1nc(-c2ccccc2)nc(-c2ccc3oc4ccccc4c3c2)n1] |
| 1-65 | 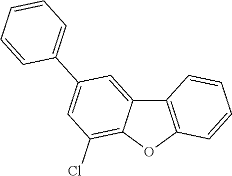 [SMILES: Clc1cc(-c2ccccc2)cc2c1oc1ccccc12] | 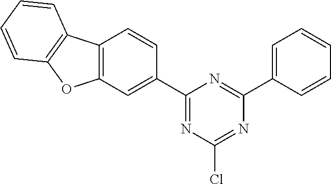 [SMILES: Clc1nc(-c2ccccc2)nc(-c2ccc3c(c2)oc2ccccc23)n1] |
| 1-66 | 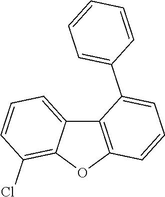 [SMILES: Clc1cccc2c1oc1cccc(-c3ccccc3)c12] | 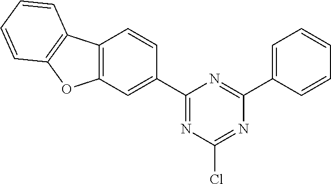 [SMILES: Clc1nc(-c2ccccc2)nc(-c2ccc3c(c2)oc2ccccc23)n1] |
| 1-67 | 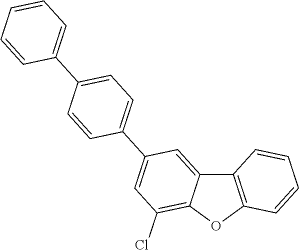 [SMILES: Clc1cc(-c2ccc(-c3ccccc3)cc2)cc2c1oc1ccccc12] | 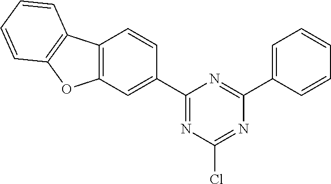 [SMILES: Clc1nc(-c2ccccc2)nc(-c2ccc3c(c2)oc2ccccc23)n1] |
| 1-68 | 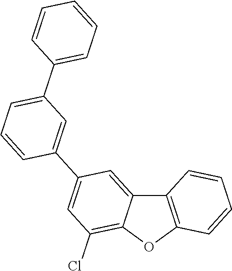 [SMILES: Clc1cc(-c2cccc(-c3ccccc3)c2)cc2c1oc1ccccc12] | 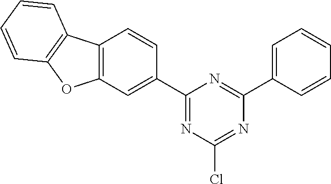 [SMILES: Clc1nc(-c2ccccc2)nc(-c2ccc3c(c2)oc2ccccc23)n1] |
| 1-77 | 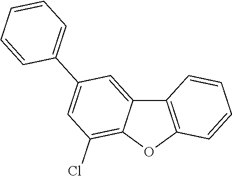 [SMILES: Clc1cc(-c2ccccc2)cc2c1oc1ccccc12] | 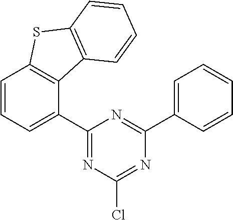 [SMILES: Clc1nc(-c2ccccc2)nc(-c2cccc3sc4ccccc4c23)n1] |
| 1-78 | 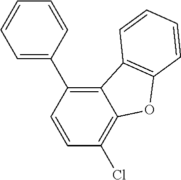 [SMILES: Clc1ccc(-c2ccccc2)c2c1oc1ccccc12] | 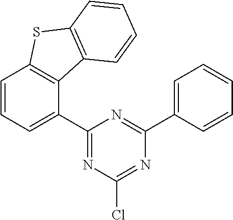 [SMILES: Clc1nc(-c2ccccc2)nc(-c2cccc3sc4ccccc4c23)n1] |
| 1-79 | 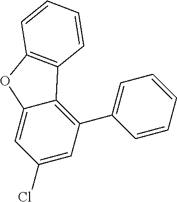 [SMILES: Clc1cc(-c2ccccc2)c2c(c1)oc1ccccc12] | 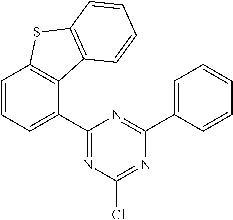 [SMILES: Clc1nc(-c2ccccc2)nc(-c2cccc3sc4ccccc4c23)n1] |
| 1-80 | 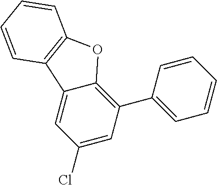 [SMILES: Clc1cc(-c2ccccc2)c2oc3ccccc3c2c1] | 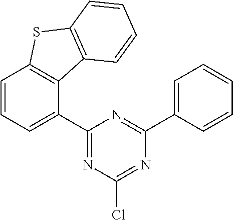 [SMILES: Clc1nc(-c2ccccc2)nc(-c2cccc3sc4ccccc4c23)n1] |
| 1-81 | 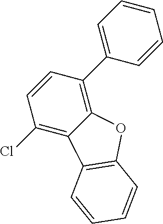 [SMILES: Clc1ccc(-c2ccccc2)c2oc3ccccc3c12] | 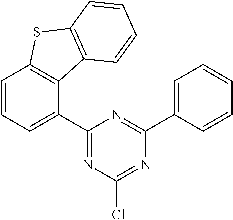 [SMILES: Clc1nc(-c2ccccc2)nc(-c2cccc3sc4ccccc4c23)n1] |
| 1-82 | 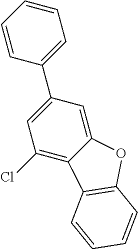 [SMILES: Clc1cc(-c2ccccc2)cc2oc3ccccc3c12] | 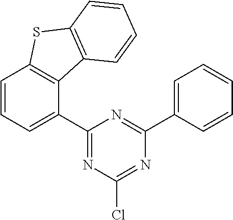 [SMILES: Clc1nc(-c2ccccc2)nc(-c2cccc3sc4ccccc4c23)n1] |
| 1-83 | 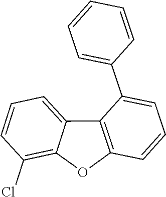 [SMILES: Clc1cccc2c1oc1cccc(-c3ccccc3)c12] | 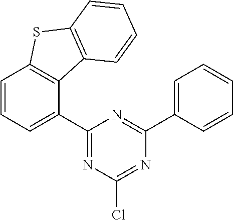 [SMILES: Clc1nc(-c2ccccc2)nc(-c2cccc3sc4ccccc4c23)n1] |
| 1-84 | 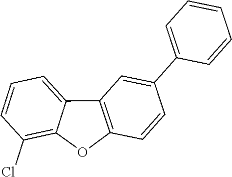 [SMILES: Clc1cccc2c1oc1ccc(-c3ccccc3)cc12] | 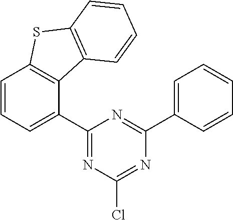 [SMILES: Clc1nc(-c2ccccc2)nc(-c2cccc3sc4ccccc4c23)n1] |
| 1-87 | 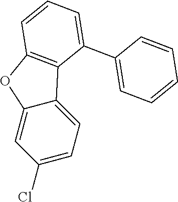 [SMILES: Clc1ccc2c(c1)oc1cccc(-c3ccccc3)c12] | 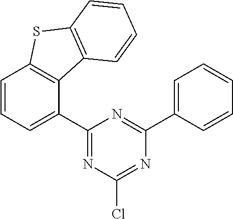 [SMILES: Clc1nc(-c2ccccc2)nc(-c2cccc3sc4ccccc4c23)n1] |
| 1-88 | 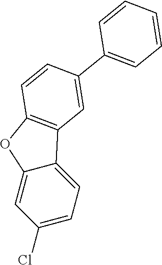 [SMILES: Clc1ccc2c(c1)oc1ccc(-c3ccccc3)cc12] | 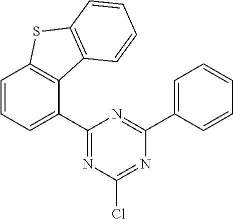 [SMILES: Clc1nc(-c2ccccc2)nc(-c2cccc3sc4ccccc4c23)n1] |
| 1-91 | 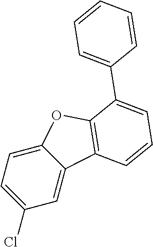 [SMILES: Clc1ccc2oc3c(-c4ccccc4)cccc3c2c1] | 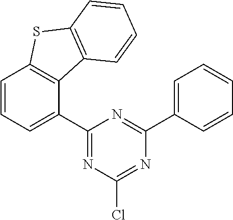 [SMILES: Clc1nc(-c2ccccc2)nc(-c2cccc3sc4ccccc4c23)n1] |
| 1-92 | 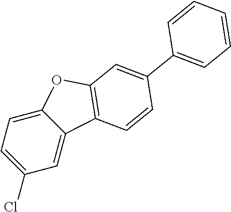 [SMILES: Clc1ccc2oc3cc(-c4ccccc4)ccc3c2c1] | 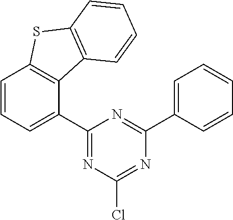 [SMILES: Clc1nc(-c2ccccc2)nc(-c2cccc3sc4ccccc4c23)n1] |
| 1-95 | 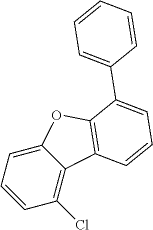 [SMILES: Clc1cccc2oc3c(-c4ccccc4)cccc3c12] | 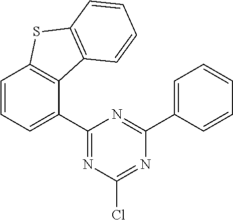 [SMILES: Clc1nc(-c2ccccc2)nc(-c2cccc3sc4ccccc4c23)n1] |
| 1-96 | 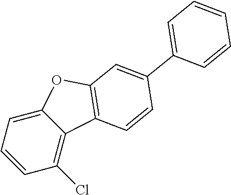 [SMILES: Clc1cccc2oc3cc(-c4ccccc4)ccc3c12] | 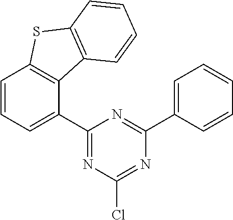 [SMILES: Clc1nc(-c2ccccc2)nc(-c2cccc3sc4ccccc4c23)n1] |
| 1-99 | 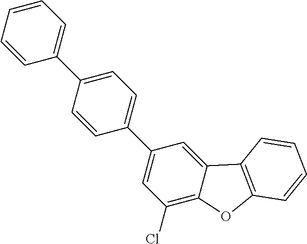 [SMILES: Clc1cc(-c2ccc(-c3ccccc3)cc2)cc2c1oc1ccccc12] | 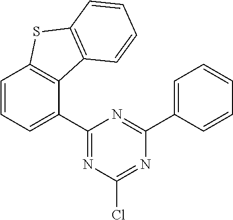 [SMILES: Clc1nc(-c2ccccc2)nc(-c2cccc3sc4ccccc4c23)n1] |
| 1-100 | 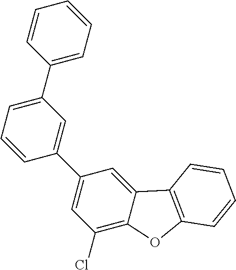 [SMILES: Clc1cc(-c2cccc(-c3ccccc3)c2)cc2c1oc1ccccc12] | 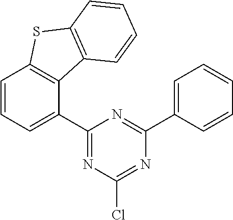 [SMILES: Clc1nc(-c2ccccc2)nc(-c2cccc3sc4ccccc4c23)n1] |
| 1-105 | 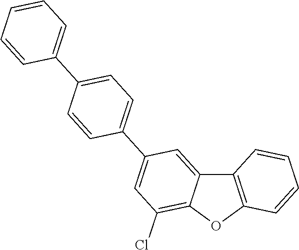 [SMILES: Clc1cc(-c2ccc(-c3ccccc3)cc2)cc2c1oc1ccccc12] | 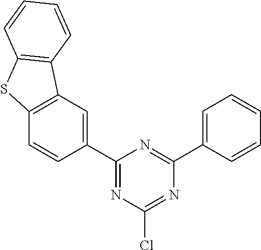 [SMILES: Clc1nc(-c2ccccc2)nc(-c2ccc3sc4ccccc4c3c2)n1] |
| 1-106 | 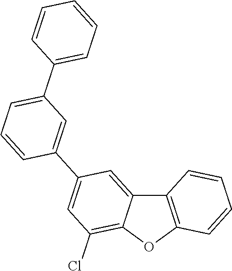 [SMILES: Clc1cc(-c2cccc(-c3ccccc3)c2)cc2c1oc1ccccc12] | 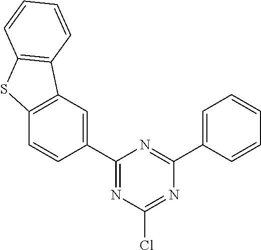 [SMILES: Clc1nc(-c2ccccc2)nc(-c2ccc3sc4ccccc4c3c2)n1] |
| 1-109 | 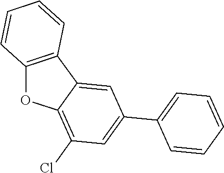 [SMILES: Clc1cc(-c2ccccc2)cc2c1oc1ccccc12] | 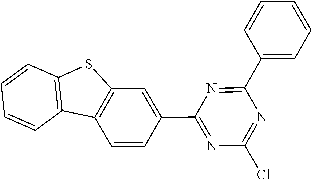 [SMILES: Clc1nc(-c2ccccc2)nc(-c2ccc3c(c2)sc2ccccc23)n1] |
| 1-121 | 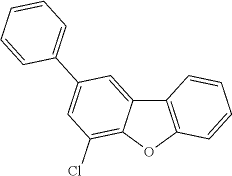 [SMILES: Clc1cc(-c2ccccc2)cc2c1oc1ccccc12] | 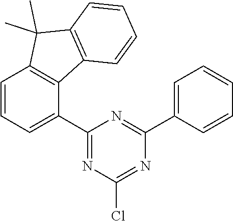 [SMILES: CC1(C)c2ccccc2-c2c(-c3nc(Cl)nc(-c4ccccc4)n3)cccc21] |
| 1-122 | 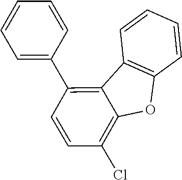 [SMILES: Clc1ccc(-c2ccccc2)c2c1oc1ccccc12] | 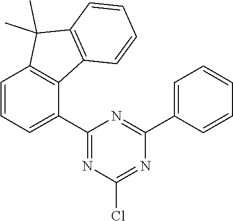 [SMILES: CC1(C)c2ccccc2-c2c(-c3nc(Cl)nc(-c4ccccc4)n3)cccc21] |
| 1-123 | 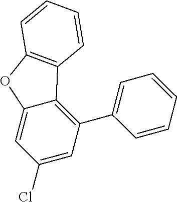 [SMILES: Clc1cc(-c2ccccc2)c2c(c1)oc1ccccc12] | 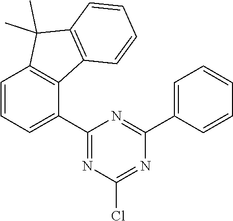 [SMILES: CC1(C)c2ccccc2-c2c(-c3nc(Cl)nc(-c4ccccc4)n3)cccc21] |
| 1-124 | 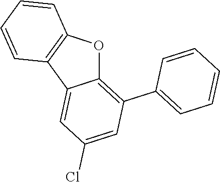 [SMILES: Clc1cc(-c2ccccc2)c2oc3ccccc3c2c1] | 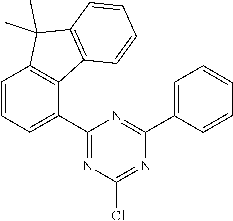 [SMILES: CC1(C)c2ccccc2-c2c(-c3nc(Cl)nc(-c4ccccc4)n3)cccc21] |
| 1-131 | 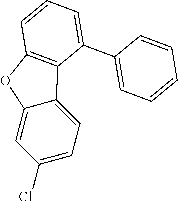 [SMILES: Clc1ccc2c(c1)oc1cccc(-c3ccccc3)c12] | 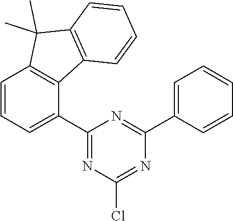 [SMILES: CC1(C)c2ccccc2-c2c(-c3nc(Cl)nc(-c4ccccc4)n3)cccc21] |
| 1-132 | 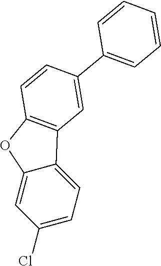 [SMILES: Clc1ccc2c(c1)oc1ccc(-c3ccccc3)cc12] | 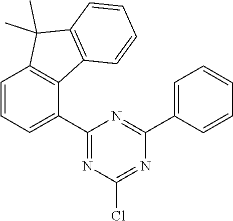 [SMILES: CC1(C)c2ccccc2-c2c(-c3nc(Cl)nc(-c4ccccc4)n3)cccc21] |
| 1-135 | 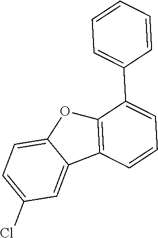 [SMILES: Clc1ccc2oc3c(-c4ccccc4)cccc3c2c1] | 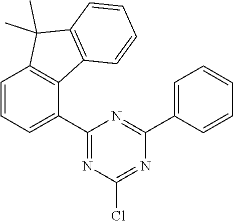 [SMILES: CC1(C)c2ccccc2-c2c(-c3nc(Cl)nc(-c4ccccc4)n3)cccc21] |
| 1-136 | 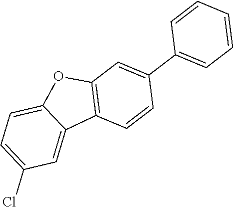 [SMILES: Clc1ccc2oc3cc(-c4ccccc4)ccc3c2c1] | 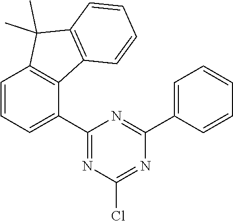 [SMILES: CC1(C)c2ccccc2-c2c(-c3nc(Cl)nc(-c4ccccc4)n3)cccc21] |
| 1-141 | 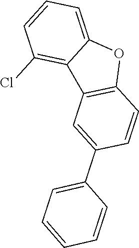 [SMILES: Clc1cccc2oc3ccc(-c4ccccc4)cc3c12] | 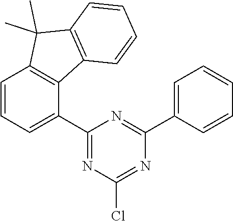 [SMILES: CC1(C)c2ccccc2-c2c(-c3nc(Cl)nc(-c4ccccc4)n3)cccc21] |
| 1-142 | 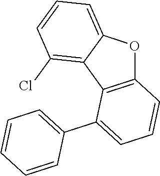 [SMILES: Clc1cccc2oc3cccc(-c4ccccc4)c3c12] | 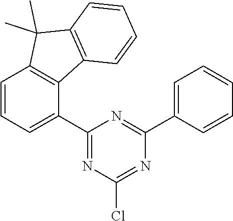 [SMILES: CC1(C)c2ccccc2-c2c(-c3nc(Cl)nc(-c4ccccc4)n3)cccc21] |
| 1-143 | 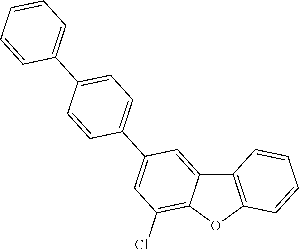 [SMILES: Clc1cc(-c2ccc(-c3ccccc3)cc2)cc2c1oc1ccccc12] | 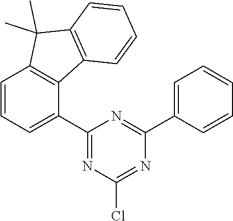 [SMILES: CC1(C)c2ccccc2-c2c(-c3nc(Cl)nc(-c4ccccc4)n3)cccc21] |
| 1-144 | 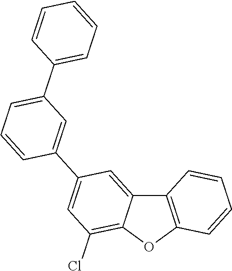 [SMILES: Clc1cc(-c2cccc(-c3ccccc3)c2)cc2c1oc1ccccc12] | 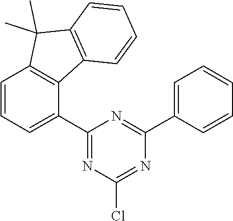 [SMILES: CC1(C)c2ccccc2-c2c(-c3nc(Cl)nc(-c4ccccc4)n3)cccc21] |
| 1-153 | 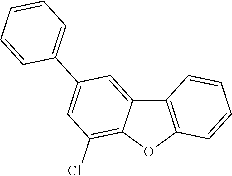 [SMILES: Clc1cc(-c2ccccc2)cc2c1oc1ccccc12] | 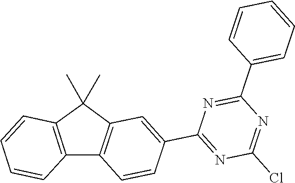 [SMILES: CC1(C)c2ccccc2-c2ccc(-c3nc(Cl)nc(-c4ccccc4)n3)cc21] |
| 1-331 | 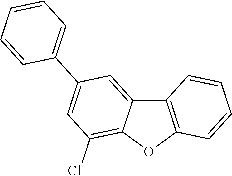 [SMILES: Clc1cc(-c2ccccc2)cc2c1oc1ccccc12] | 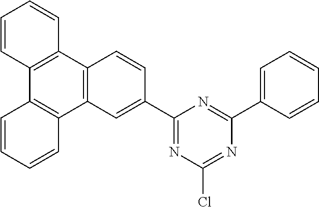 [SMILES: Clc1nc(-c2ccccc2)nc(-c2ccc3c4ccccc4c4ccccc4c3c2)n1] |
| 1-332 | 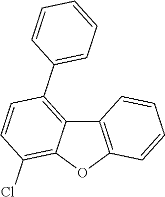 [SMILES: Clc1ccc(-c2ccccc2)c2c1oc1ccccc12] | 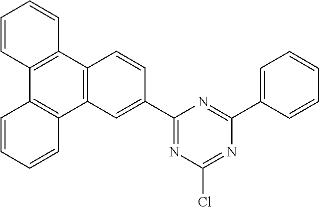 [SMILES: Clc1nc(-c2ccccc2)nc(-c2ccc3c4ccccc4c4ccccc4c3c2)n1] |
| 1-335 | 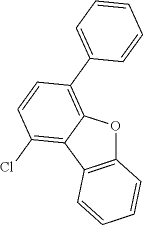 [SMILES: Clc1ccc(-c2ccccc2)c2oc3ccccc3c12] | 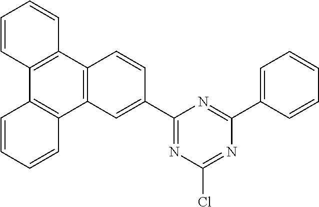 [SMILES: Clc1nc(-c2ccccc2)nc(-c2ccc3c4ccccc4c4ccccc4c3c2)n1] |
| 1-230 | 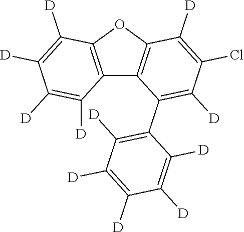 [SMILES: [2H]c1c([2H])c([2H])c(-c2c([2H])c(Cl)c([2H])c3oc4c([2H])c([2H])c([2H])c([2H])c4c23)c([2H])c1[2H]] | 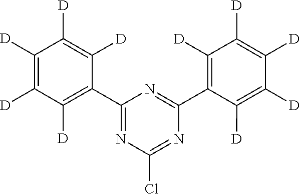 [SMILES: [2H]c1c([2H])c([2H])c(-c2nc(Cl)nc(-c3c([2H])c([2H])c([2H])c([2H])c3[2H])n2)c([2H])c1[2H]] |
| 1-242 | 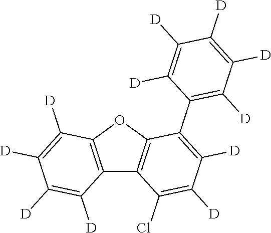 [SMILES: [2H]c1c([2H])c([2H])c(-c2c([2H])c([2H])c(Cl)c3c2oc2c([2H])c([2H])c([2H])c([2H])c23)c([2H])c1[2H]] | 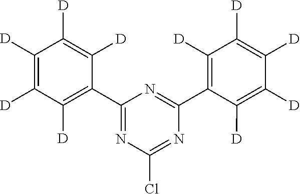 [SMILES: [2H]c1c([2H])c([2H])c(-c2nc(Cl)nc(-c3c([2H])c([2H])c([2H])c([2H])c3[2H])n2)c([2H])c1[2H]] |
| 1-245 | 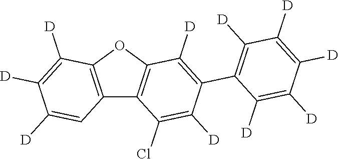 [SMILES: [2H]c1cc2c(oc3c([2H])c(-c4c([2H])c([2H])c([2H])c([2H])c4[2H])c([2H])c(Cl)c32)c([2H])c1[2H]] | 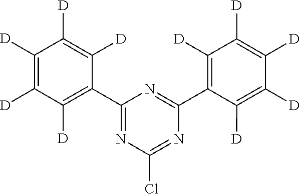 [SMILES: [2H]c1c([2H])c([2H])c(-c2nc(Cl)nc(-c3c([2H])c([2H])c([2H])c([2H])c3[2H])n2)c([2H])c1[2H]] |
| 1-248 | 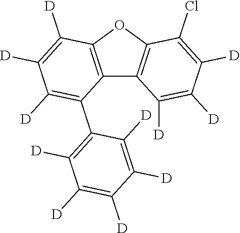 [SMILES: [2H]c1c([2H])c([2H])c(-c2c([2H])c([2H])c([2H])c3oc4c(Cl)c([2H])c([2H])c([2H])c4c23)c([2H])c1[2H]] | 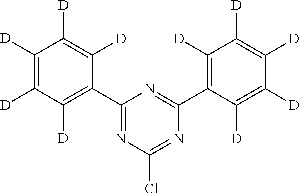 [SMILES: [2H]c1c([2H])c([2H])c(-c2nc(Cl)nc(-c3c([2H])c([2H])c([2H])c([2H])c3[2H])n2)c([2H])c1[2H]] |
| 1-266 | 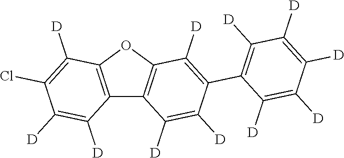 [SMILES: [2H]c1c([2H])c([2H])c(-c2c([2H])c([2H])c3c(oc4c([2H])c(Cl)c([2H])c([2H])c43)c2[2H])c([2H])c1[2H]] | 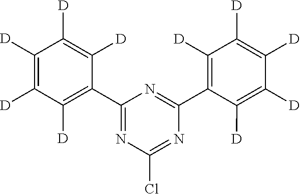 [SMILES: [2H]c1c([2H])c([2H])c(-c2nc(Cl)nc(-c3c([2H])c([2H])c([2H])c([2H])c3[2H])n2)c([2H])c1[2H]] |
| 1-269 | 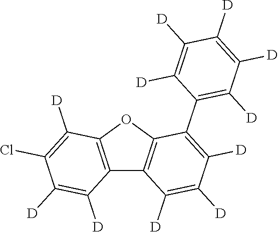 [SMILES: [2H]c1c([2H])c([2H])c(-c2c([2H])c([2H])c([2H])c3c2oc2c([2H])c(Cl)c([2H])c([2H])c23)c([2H])c1[2H]] | 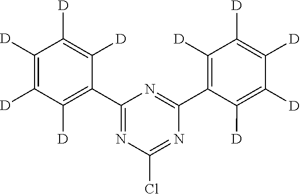 [SMILES: [2H]c1c([2H])c([2H])c(-c2nc(Cl)nc(-c3c([2H])c([2H])c([2H])c([2H])c3[2H])n2)c([2H])c1[2H]] |

| Com- | |
|---|---|
| pound | Target Compound |
| No. | (Yield %) |
| 1-2 | 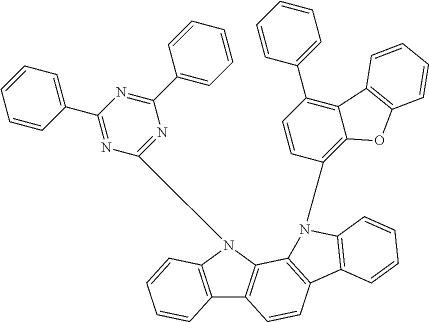 [SMILES: c1ccc(-c2nc(-c3ccccc3)nc(-n3c4ccccc4c4ccc5c6ccccc6n(-c6ccc(-c7ccccc7)c7c6oc6ccccc67)c5c43)n2)cc1] |
| | 54 % |
| 1-3 | 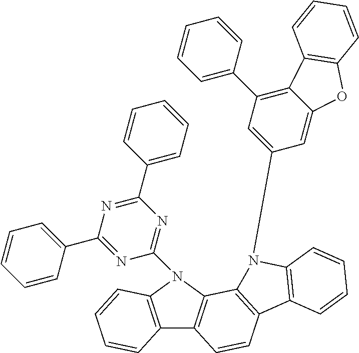 [SMILES: c1ccc(-c2nc(-c3ccccc3)nc(-n3c4ccccc4c4ccc5c6ccccc6n(-c6cc(-c7ccccc7)c7c(c6)oc6ccccc67)c5c43)n2)cc1] |
| | 55% |
| 1-4 | 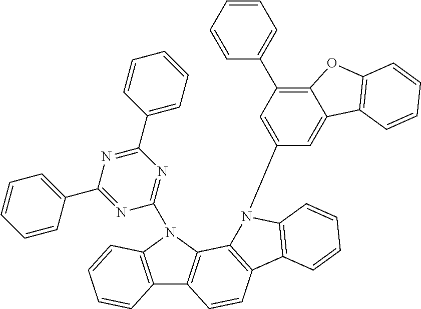 [SMILES: c1ccc(-c2nc(-c3ccccc3)nc(-n3c4ccccc4c4ccc5c6ccccc6n(-c6cc(-c7ccccc7)c7oc8ccccc8c7c6)c5c43)n2)cc1] |
| | 52% |
| 1-5 | 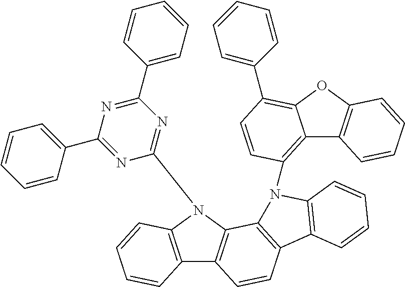 [SMILES: c1ccc(-c2nc(-c3ccccc3)nc(-n3c4ccccc4c4ccc5c6ccccc6n(-c6ccc(-c7ccccc7)c7oc8ccccc8c67)c5c43)n2)cc1] |
| | 55% |
| 1-6 | 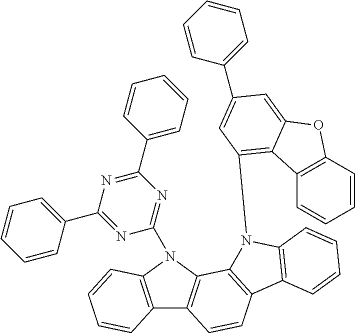 [SMILES: c1ccc(-c2cc(-n3c4ccccc4c4ccc5c6ccccc6n(-c6nc(-c7ccccc7)nc(-c7ccccc7)n6)c5c43)c3c(c2)oc2ccccc23)cc1] |
| | 53% |
| 1-11 | 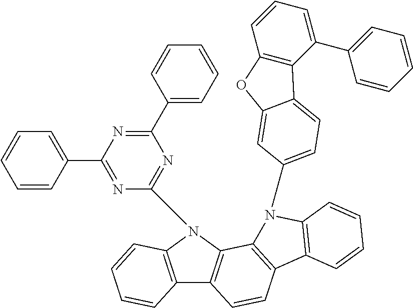 [SMILES: c1ccc(-c2nc(-c3ccccc3)nc(-n3c4ccccc4c4ccc5c6ccccc6n(-c6ccc7c(c6)oc6cccc(-c8ccccc8)c67)c5c43)n2)cc1] |
| | 53% |
| 1-12 | 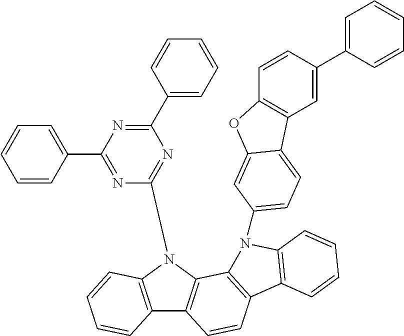 [SMILES: c1ccc(-c2ccc3oc4cc(-n5c6ccccc6c6ccc7c8ccccc8n(-c8nc(-c9ccccc9)nc(-c9ccccc9)n8)c7c65)ccc4c3c2)cc1] |
| | 55% |
| 1-13 | 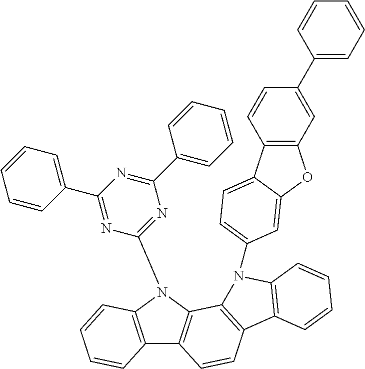 [SMILES: c1ccc(-c2ccc3c(c2)oc2cc(-n4c5ccccc5c5ccc6c7ccccc7n(-c7nc(-c8ccccc8)nc(-c8ccccc8)n7)c6c54)ccc23)cc1] |
| | 54% |
| 1-14 | 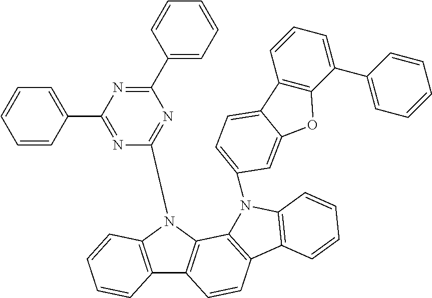 [SMILES: c1ccc(-c2nc(-c3ccccc3)nc(-n3c4ccccc4c4ccc5c6ccccc6n(-c6ccc7c(c6)oc6c(-c8ccccc8)cccc67)c5c43)n2)cc1] |
| | 53% |
| 1-15 | 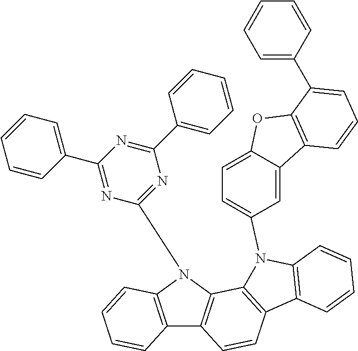 [SMILES: c1ccc(-c2nc(-c3ccccc3)nc(-n3c4ccccc4c4ccc5c6ccccc6n(-c6ccc7oc8c(-c9ccccc9)cccc8c7c6)c5c43)n2)cc1] |
| | 55% |
| 1-17 | 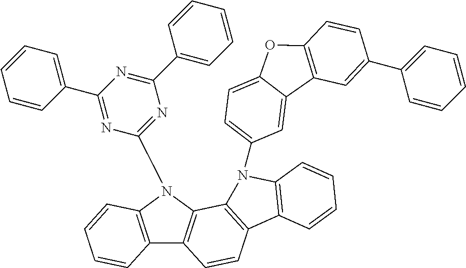 [SMILES: c1ccc(-c2ccc3oc4ccc(-n5c6ccccc6c6ccc7c8ccccc8n(-c8nc(-c9ccccc9)nc(-c9ccccc9)n8)c7c65)cc4c3c2)cc1] |
| | 53% |
| 1-21 | 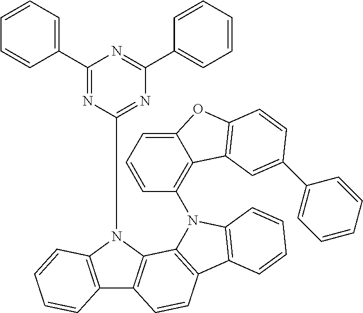 [SMILES: c1ccc(-c2ccc3oc4cccc(-n5c6ccccc6c6ccc7c8ccccc8n(-c8nc(-c9ccccc9)nc(-c9ccccc9)n8)c7c65)c4c3c2)cc1] |
| | 49% |
| 1-22 | 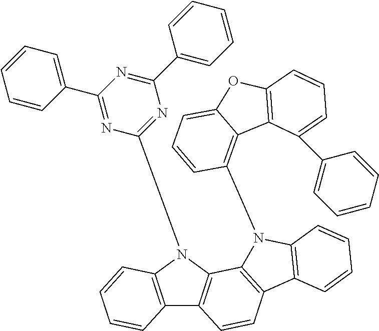 [SMILES: c1ccc(-c2nc(-c3ccccc3)nc(-n3c4ccccc4c4ccc5c6ccccc6n(-c6cccc7oc8cccc(-c9ccccc9)c8c67)c5c43)n2)cc1] |
| | 43% |
| 1-23 | 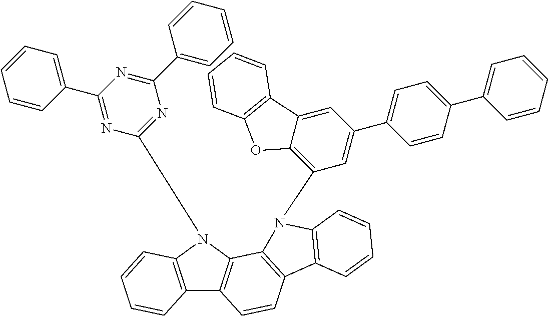 [SMILES: c1ccc(-c2ccc(-c3cc(-n4c5ccccc5c5ccc6c7ccccc7n(-c7nc(-c8ccccc8)nc(-c8ccccc8)n7)c6c54)c4oc5ccccc5c4c3)cc2)cc1] |
| | 51% |
| 1-24 | 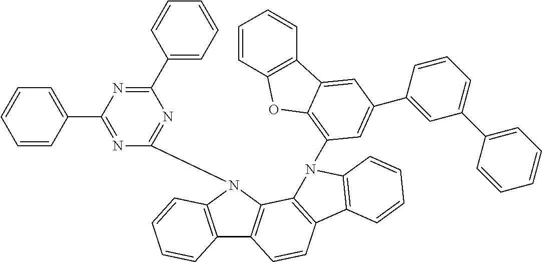 [SMILES: c1ccc(-c2cccc(-c3cc(-n4c5ccccc5c5ccc6c7ccccc7n(-c7nc(-c8ccccc8)nc(-c8ccccc8)n7)c6c54)c4oc5ccccc5c4c3)c2)cc1] |
| | 55% |
| 1-27 | 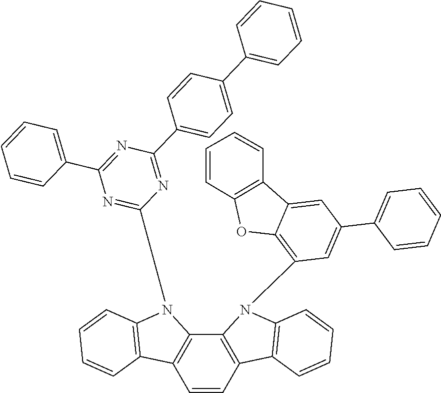 [SMILES: c1ccc(-c2ccc(-c3nc(-c4ccccc4)nc(-n4c5ccccc5c5ccc6c7ccccc7n(-c7cc(-c8ccccc8)cc8c7oc7ccccc78)c6c54)n3)cc2)cc1] |
| | 51% |
| 1-33 | 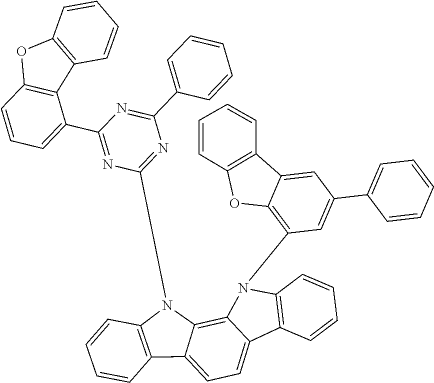 [SMILES: c1ccc(-c2cc(-n3c4ccccc4c4ccc5c6ccccc6n(-c6nc(-c7ccccc7)nc(-c7cccc8oc9ccccc9c78)n6)c5c43)c3oc4ccccc4c3c2)cc1] |
| | 51% |
| 1-34 | 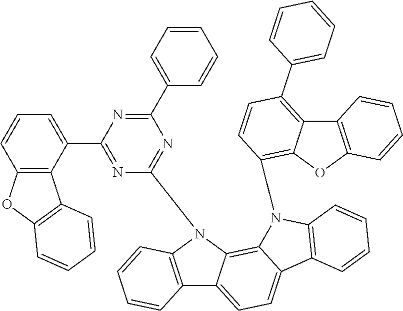 [SMILES: c1ccc(-c2nc(-c3cccc4oc5ccccc5c34)nc(-n3c4ccccc4c4ccc5c6ccccc6n(-c6ccc(-c7ccccc7)c7c6oc6ccccc67)c5c43)n2)cc1] |
| | 52% |
| 1-35 | 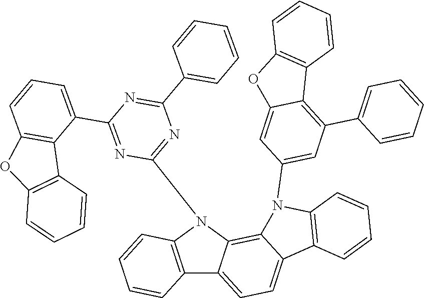 [SMILES: c1ccc(-c2nc(-c3cccc4oc5ccccc5c34)nc(-n3c4ccccc4c4ccc5c6ccccc6n(-c6cc(-c7ccccc7)c7c(c6)oc6ccccc67)c5c43)n2)cc1] |
| | 49% |
| 1-36 | 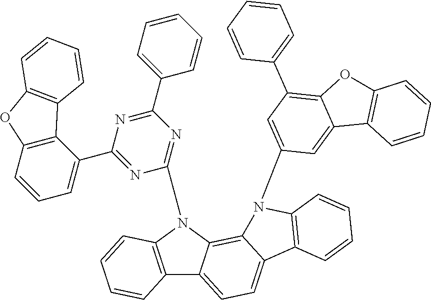 [SMILES: c1ccc(-c2nc(-c3cccc4oc5ccccc5c34)nc(-n3c4ccccc4c4ccc5c6ccccc6n(-c6cc(-c7ccccc7)c7oc8ccccc8c7c6)c5c43)n2)cc1] |
| | 49% |
| 1-37 | 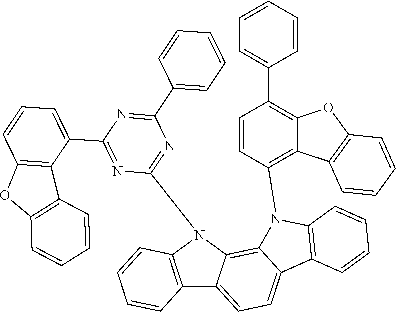 [SMILES: c1ccc(-c2nc(-c3cccc4oc5ccccc5c34)nc(-n3c4ccccc4c4ccc5c6ccccc6n(-c6ccc(-c7ccccc7)c7oc8ccccc8c67)c5c43)n2)cc1] |
| | 47% |
| 1-38 | 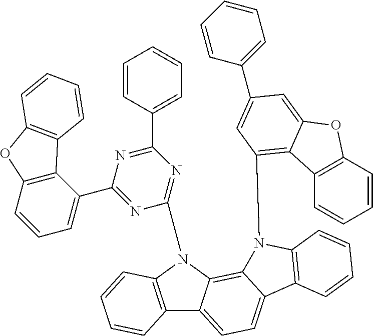 [SMILES: c1ccc(-c2cc(-n3c4ccccc4c4ccc5c6ccccc6n(-c6nc(-c7ccccc7)nc(-c7cccc8oc9ccccc9c78)n6)c5c43)c3c(c2)oc2ccccc23)cc1] |
| | 48% |
| 1-43 | 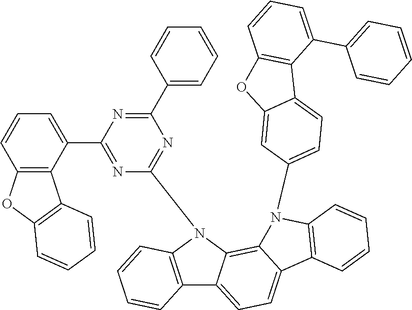 [SMILES: c1ccc(-c2nc(-c3cccc4oc5ccccc5c34)nc(-n3c4ccccc4c4ccc5c6ccccc6n(-c6ccc7c(c6)oc6cccc(-c8ccccc8)c67)c5c43)n2)cc1] |
| | 50% |
| 1-44 | 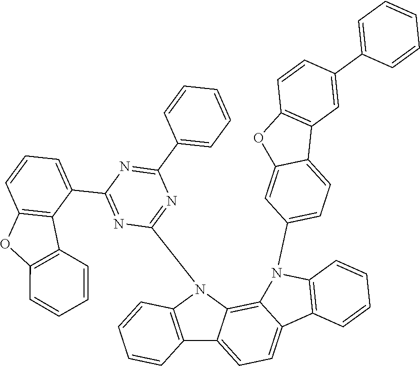 [SMILES: c1ccc(-c2ccc3oc4cc(-n5c6ccccc6c6ccc7c8ccccc8n(-c8nc(-c9ccccc9)nc(-c9cccc%10oc%11ccccc%11c9%10)n8)c7c65)ccc4c3c2)cc1] |
| | 51% |
| 1-47 | 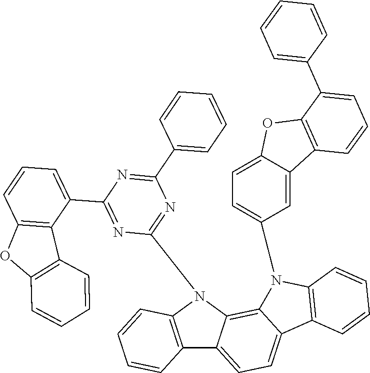 [SMILES: c1ccc(-c2nc(-c3cccc4oc5ccccc5c34)nc(-n3c4ccccc4c4ccc5c6ccccc6n(-c6ccc7oc8c(-c9ccccc9)cccc8c7c6)c5c43)n2)cc1] |
| | 48% |
| 1-48 | 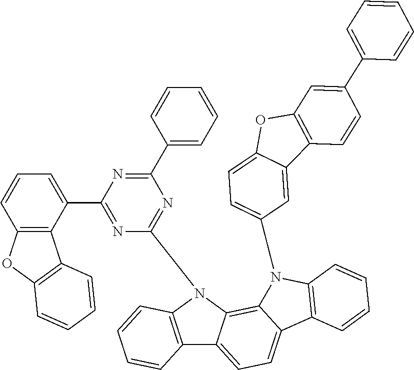 [SMILES: c1ccc(-c2ccc3c(c2)oc2ccc(-n4c5ccccc5c5ccc6c7ccccc7n(-c7nc(-c8ccccc8)nc(-c8cccc9oc%10ccccc%10c89)n7)c6c54)cc23)cc1] |
| | 49% |
| 1-51 | 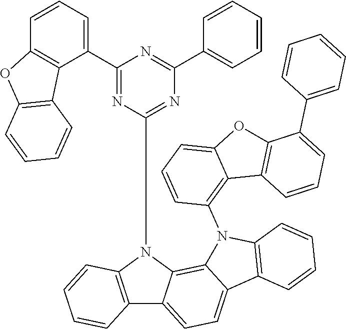 [SMILES: c1ccc(-c2nc(-c3cccc4oc5ccccc5c34)nc(-n3c4ccccc4c4ccc5c6ccccc6n(-c6cccc7oc8c(-c9ccccc9)cccc8c67)c5c43)n2)cc1] |
| | 45% |
| 1-52 | 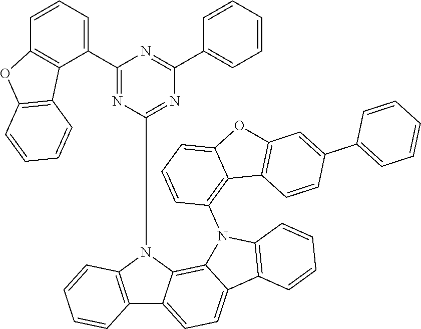 [SMILES: c1ccc(-c2ccc3c(c2)oc2cccc(-n4c5ccccc5c5ccc6c7ccccc7n(-c7nc(-c8ccccc8)nc(-c8cccc9oc%10ccccc%10c89)n7)c6c54)c23)cc1] |
| | 47% |
| 1-55 | 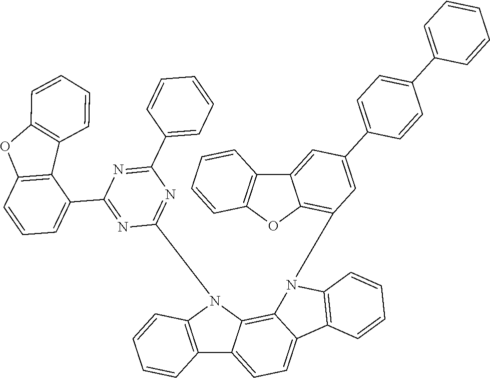 [SMILES: c1ccc(-c2ccc(-c3cc(-n4c5ccccc5c5ccc6c7ccccc7n(-c7nc(-c8ccccc8)nc(-c8cccc9oc%10ccccc%10c89)n7)c6c54)c4oc5ccccc5c4c3)cc2)cc1] |
| | 47% |
| 1-56 | 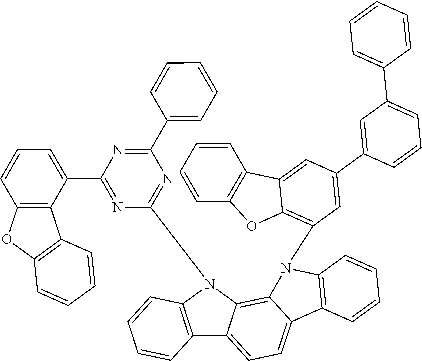 [SMILES: c1ccc(-c2cccc(-c3cc(-n4c5ccccc5c5ccc6c7ccccc7n(-c7nc(-c8ccccc8)nc(-c8cccc9oc%10ccccc%10c89)n7)c6c54)c4oc5ccccc5c4c3)c2)cc1] |
| | 48% |
| 1-61 | 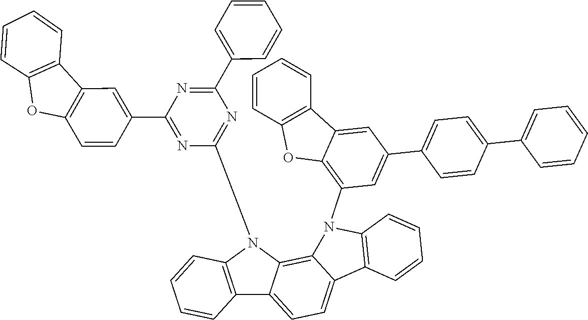 [SMILES: c1ccc(-c2ccc(-c3cc(-n4c5ccccc5c5ccc6c7ccccc7n(-c7nc(-c8ccccc8)nc(-c8ccc9oc%10ccccc%10c9c8)n7)c6c54)c4oc5ccccc5c4c3)cc2)cc1] |
| | 48% |
| 1-62 | 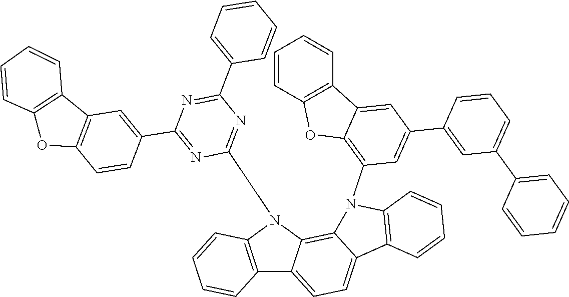 [SMILES: c1ccc(-c2cccc(-c3cc(-n4c5ccccc5c5ccc6c7ccccc7n(-c7nc(-c8ccccc8)nc(-c8ccc9oc%10ccccc%10c9c8)n7)c6c54)c4oc5ccccc5c4c3)c2)cc1] |
| | 49% |
| 1-65 | 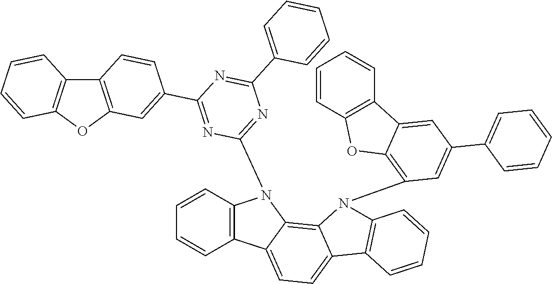 [SMILES: c1ccc(-c2cc(-n3c4ccccc4c4ccc5c6ccccc6n(-c6nc(-c7ccccc7)nc(-c7ccc8c(c7)oc7ccccc78)n6)c5c43)c3oc4ccccc4c3c2)cc1] |
| | 53% |
| 1-66 | 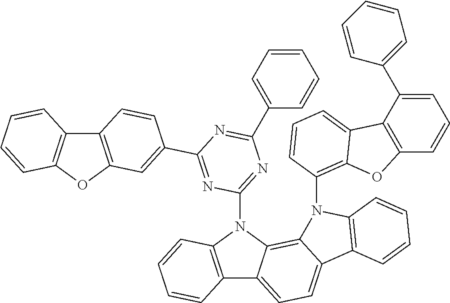 [SMILES: c1ccc(-c2nc(-c3ccc4c(c3)oc3ccccc34)nc(-n3c4ccccc4c4ccc5c6ccccc6n(-c6cccc7c6oc6cccc(-c8ccccc8)c67)c5c43)n2)cc1] |
| | 52% |
| 1-67 | 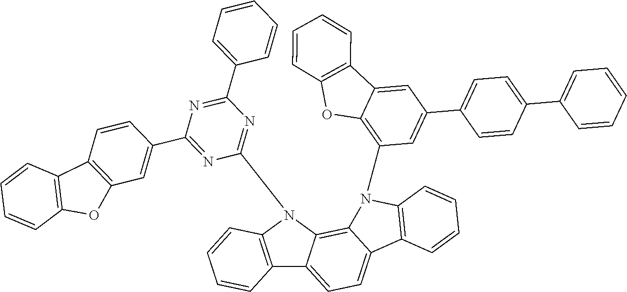 [SMILES: c1ccc(-c2ccc(-c3cc(-n4c5ccccc5c5ccc6c7ccccc7n(-c7nc(-c8ccccc8)nc(-c8ccc9c(c8)oc8ccccc89)n7)c6c54)c4oc5ccccc5c4c3)cc2)cc1] |
| | 47% |
| 1-68 | 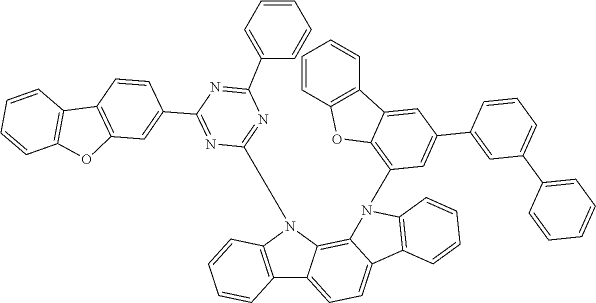 [SMILES: c1ccc(-c2cccc(-c3cc(-n4c5ccccc5c5ccc6c7ccccc7n(-c7nc(-c8ccccc8)nc(-c8ccc9c(c8)oc8ccccc89)n7)c6c54)c4oc5ccccc5c4c3)c2)cc1] |
| | 47% |
| 1-77 | 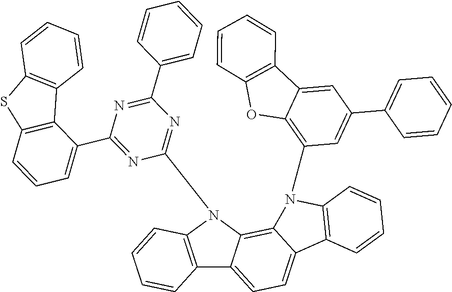 [SMILES: c1ccc(-c2cc(-n3c4ccccc4c4ccc5c6ccccc6n(-c6nc(-c7ccccc7)nc(-c7cccc8sc9ccccc9c78)n6)c5c43)c3oc4ccccc4c3c2)cc1] |
| | 52% |
| 1-78 | 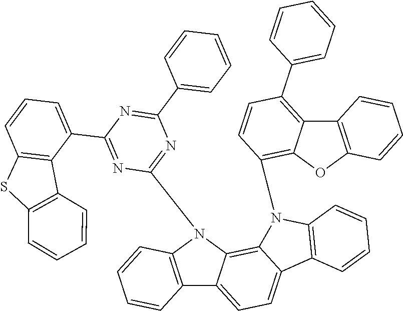 [SMILES: c1ccc(-c2nc(-c3cccc4sc5ccccc5c34)nc(-n3c4ccccc4c4ccc5c6ccccc6n(-c6ccc(-c7ccccc7)c7c6oc6ccccc67)c5c43)n2)cc1] |
| | 51% |
| 1-79 | 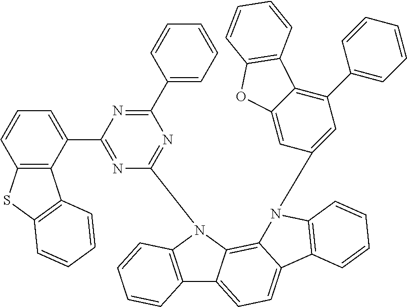 [SMILES: c1ccc(-c2nc(-c3cccc4sc5ccccc5c34)nc(-n3c4ccccc4c4ccc5c6ccccc6n(-c6cc(-c7ccccc7)c7c(c6)oc6ccccc67)c5c43)n2)cc1] |
| | 51% |
| 1-80 | 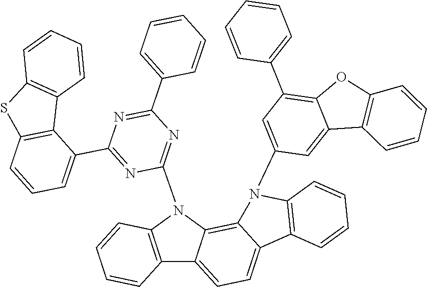 [SMILES: c1ccc(-c2nc(-c3cccc4sc5ccccc5c34)nc(-n3c4ccccc4c4ccc5c6ccccc6n(-c6cc(-c7ccccc7)c7oc8ccccc8c7c6)c5c43)n2)cc1] |
| | 52% |
| 1-81 | 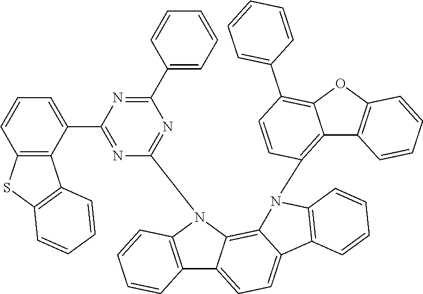 [SMILES: c1ccc(-c2nc(-c3cccc4sc5ccccc5c34)nc(-n3c4ccccc4c4ccc5c6ccccc6n(-c6ccc(-c7ccccc7)c7oc8ccccc8c67)c5c43)n2)cc1] |
| | 53% |
| 1-82 | 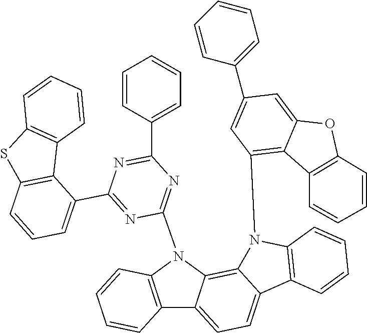 [SMILES: c1ccc(-c2cc(-n3c4ccccc4c4ccc5c6ccccc6n(-c6nc(-c7ccccc7)nc(-c7cccc8sc9ccccc9c78)n6)c5c43)c3c(c2)oc2ccccc23)cc1] |
| | 51% |
| 1-83 | 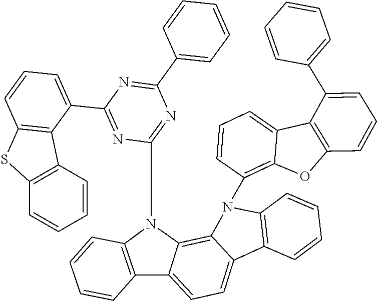 [SMILES: c1ccc(-c2nc(-c3cccc4sc5ccccc5c34)nc(-n3c4ccccc4c4ccc5c6ccccc6n(-c6cccc7c6oc6cccc(-c8ccccc8)c67)c5c43)n2)cc1] |
| | 53% |
| 1-84 | 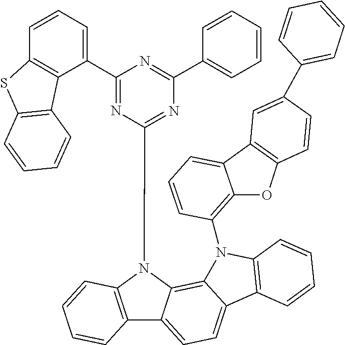 [SMILES: c1ccc(-c2ccc3oc4c(-n5c6ccccc6c6ccc7c8ccccc8n(-c8nc(-c9ccccc9)nc(-c9cccc%10sc%11ccccc%11c9%10)n8)c7c65)cccc4c3c2)cc1] |
| | 53% |
| 1-87 | 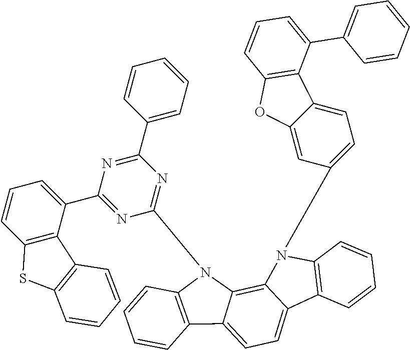 [SMILES: c1ccc(-c2nc(-c3cccc4sc5ccccc5c34)nc(-n3c4ccccc4c4ccc5c6ccccc6n(-c6ccc7c(c6)oc6cccc(-c8ccccc8)c67)c5c43)n2)cc1] |
| | 56% |
| 1-88 | 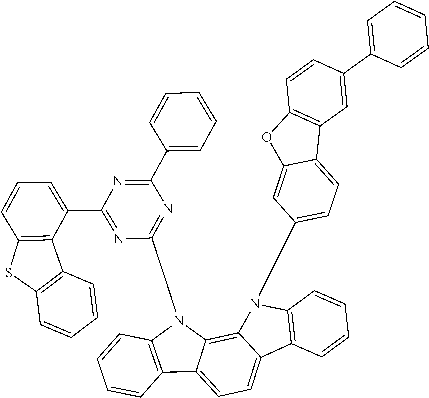 [SMILES: c1ccc(-c2ccc3oc4cc(-n5c6ccccc6c6ccc7c8ccccc8n(-c8nc(-c9ccccc9)nc(-c9cccc%10sc%11ccccc%11c9%10)n8)c7c65)ccc4c3c2)cc1] |
| | 51% |
| 1-91 | 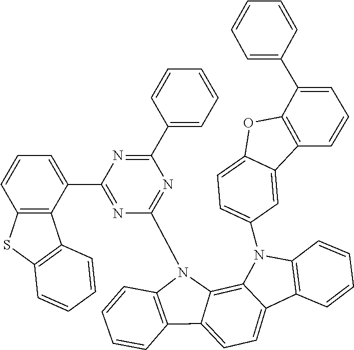 [SMILES: c1ccc(-c2nc(-c3cccc4sc5ccccc5c34)nc(-n3c4ccccc4c4ccc5c6ccccc6n(-c6ccc7oc8c(-c9ccccc9)cccc8c7c6)c5c43)n2)cc1] |
| | 55% |
| 1-92 | 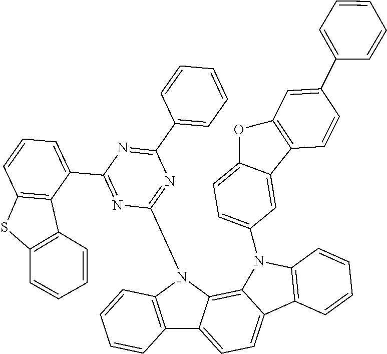 [SMILES: c1ccc(-c2ccc3c(c2)oc2ccc(-n4c5ccccc5c5ccc6c7ccccc7n(-c7nc(-c8ccccc8)nc(-c8cccc9sc%10ccccc%10c89)n7)c6c54)cc23)cc1] |
| | 51% |
| 1-95 | 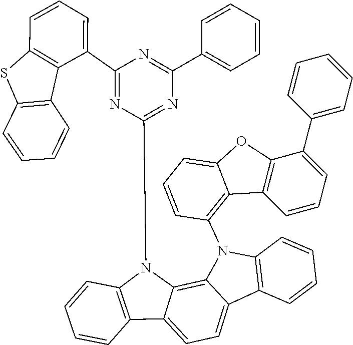 [SMILES: c1ccc(-c2nc(-c3cccc4sc5ccccc5c34)nc(-n3c4ccccc4c4ccc5c6ccccc6n(-c6cccc7oc8c(-c9ccccc9)cccc8c67)c5c43)n2)cc1] |
| | 49% |
| 1-96 | 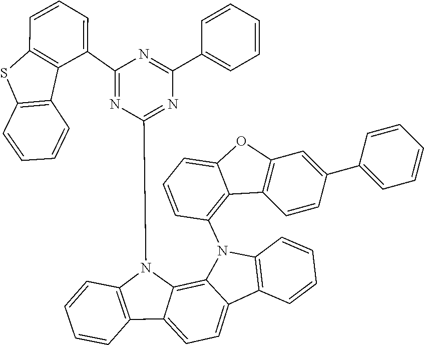 [SMILES: c1ccc(-c2ccc3c(c2)oc2cccc(-n4c5ccccc5c5ccc6c7ccccc7n(-c7nc(-c8ccccc8)nc(-c8cccc9sc%10ccccc%10c89)n7)c6c54)c23)cc1] |
| | 51% |
| 1-99 | 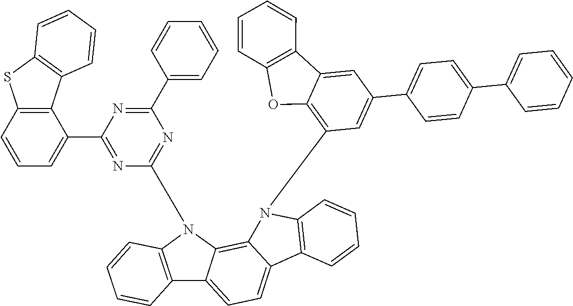 [SMILES: c1ccc(-c2ccc(-c3cc(-n4c5ccccc5c5ccc6c7ccccc7n(-c7nc(-c8ccccc8)nc(-c8cccc9sc%10ccccc%10c89)n7)c6c54)c4oc5ccccc5c4c3)cc2)cc1] |
| | 47% |
| 1-100 | 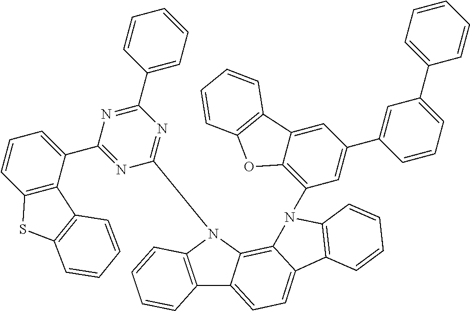 [SMILES: c1ccc(-c2cccc(-c3cc(-n4c5ccccc5c5ccc6c7ccccc7n(-c7nc(-c8ccccc8)nc(-c8cccc9sc%10ccccc%10c89)n7)c6c54)c4oc5ccccc5c4c3)c2)cc1] |
| | 46% |
| 1-105 | 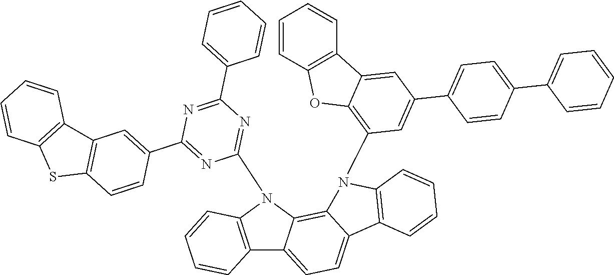 [SMILES: c1ccc(-c2ccc(-c3cc(-n4c5ccccc5c5ccc6c7ccccc7n(-c7nc(-c8ccccc8)nc(-c8ccc9sc%10ccccc%10c9c8)n7)c6c54)c4oc5ccccc5c4c3)cc2)cc1] |
| | 49% |
| 1-106 | 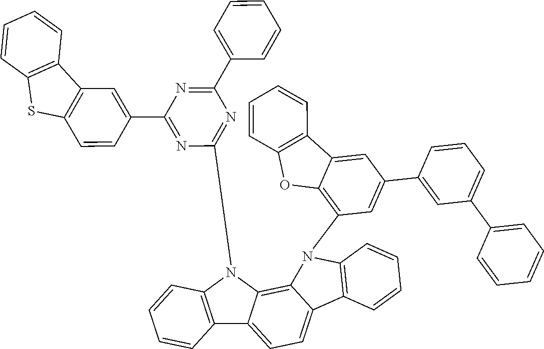 [SMILES: c1ccc(-c2cccc(-c3cc(-n4c5ccccc5c5ccc6c7ccccc7n(-c7nc(-c8ccccc8)nc(-c8ccc9sc%10ccccc%10c9c8)n7)c6c54)c4oc5ccccc5c4c3)c2)cc1] |
| | 47% |
| 1-109 | 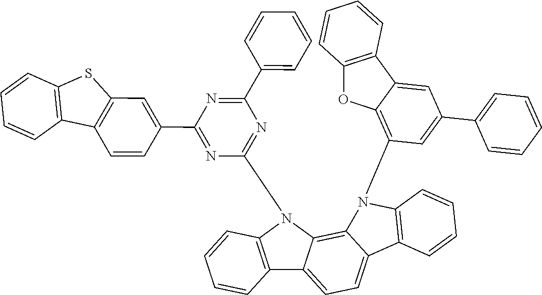 [SMILES: c1ccc(-c2cc(-n3c4ccccc4c4ccc5c6ccccc6n(-c6nc(-c7ccccc7)nc(-c7ccc8c(c7)sc7ccccc78)n6)c5c43)c3oc4ccccc4c3c2)cc1] |
| | 51% |
| 1-121 | 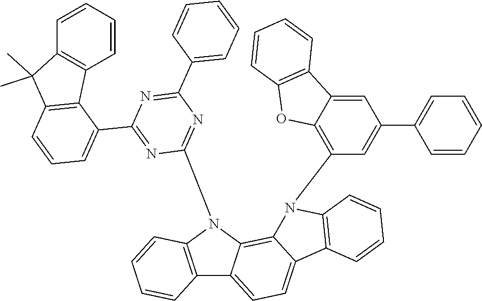 [SMILES: CC1(C)c2ccccc2-c2c(-c3nc(-c4ccccc4)nc(-n4c5ccccc5c5ccc6c7ccccc7n(-c7cc(-c8ccccc8)cc8c7oc7ccccc78)c6c54)n3)cccc21] |
| | 50% |
| 1-122 | 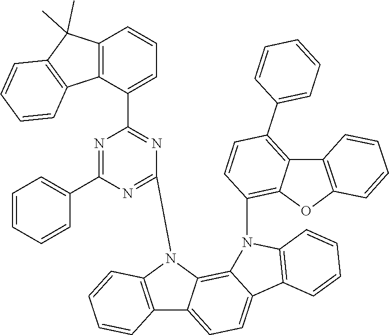 [SMILES: CC1(C)c2ccccc2-c2c(-c3nc(-c4ccccc4)nc(-n4c5ccccc5c5ccc6c7ccccc7n(-c7ccc(-c8ccccc8)c8c7oc7ccccc78)c6c54)n3)cccc21] |
| | 51% |
| 1-123 | 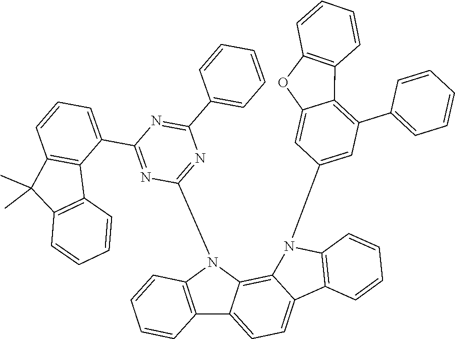 [SMILES: CC1(C)c2ccccc2-c2c(-c3nc(-c4ccccc4)nc(-n4c5ccccc5c5ccc6c7ccccc7n(-c7cc(-c8ccccc8)c8c(c7)oc7ccccc78)c6c54)n3)cccc21] |
| | 53% |
| 1-124 | 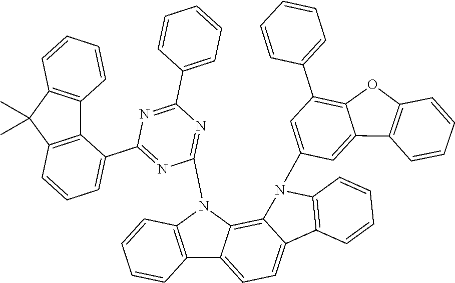 [SMILES: CC1(C)c2ccccc2-c2c(-c3nc(-c4ccccc4)nc(-n4c5ccccc5c5ccc6c7ccccc7n(-c7cc(-c8ccccc8)c8oc9ccccc9c8c7)c6c54)n3)cccc21] |
| | 55% |
| 1-131 | 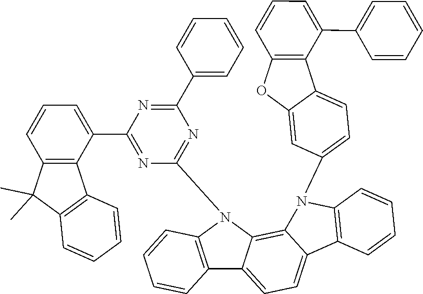 [SMILES: CC1(C)c2ccccc2-c2c(-c3nc(-c4ccccc4)nc(-n4c5ccccc5c5ccc6c7ccccc7n(-c7ccc8c(c7)oc7cccc(-c9ccccc9)c78)c6c54)n3)cccc21] |
| | 51% |
| 1-132 | 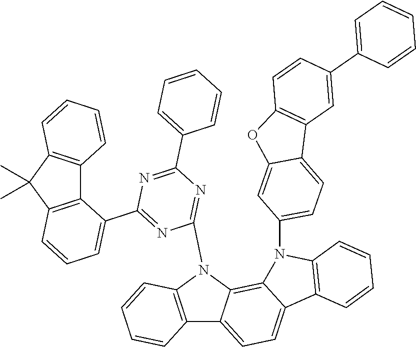 [SMILES: CC1(C)c2ccccc2-c2c(-c3nc(-c4ccccc4)nc(-n4c5ccccc5c5ccc6c7ccccc7n(-c7ccc8c(c7)oc7ccc(-c9ccccc9)cc78)c6c54)n3)cccc21] |
| | 43% |
| 1-135 | 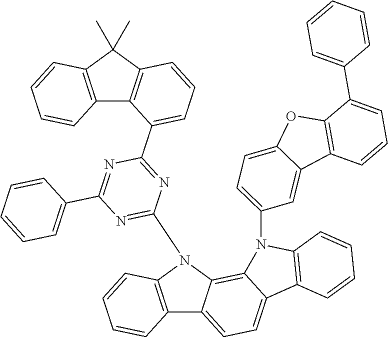 [SMILES: CC1(C)c2ccccc2-c2c(-c3nc(-c4ccccc4)nc(-n4c5ccccc5c5ccc6c7ccccc7n(-c7ccc8oc9c(-c%10ccccc%10)cccc9c8c7)c6c54)n3)cccc21] |
| | 47% |
| 1-136 | 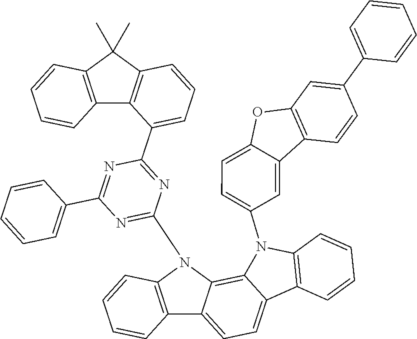 [SMILES: CC1(C)c2ccccc2-c2c(-c3nc(-c4ccccc4)nc(-n4c5ccccc5c5ccc6c7ccccc7n(-c7ccc8oc9cc(-c%10ccccc%10)ccc9c8c7)c6c54)n3)cccc21] |
| | 45% |
| 1-141 | 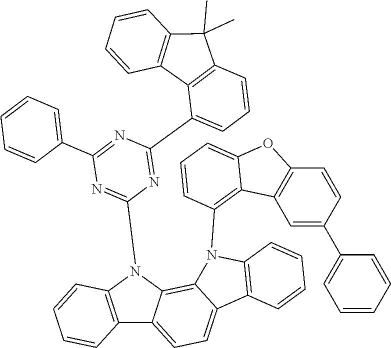 [SMILES: CC1(C)c2ccccc2-c2c(-c3nc(-c4ccccc4)nc(-n4c5ccccc5c5ccc6c7ccccc7n(-c7cccc8oc9ccc(-c%10ccccc%10)cc9c78)c6c54)n3)cccc21] |
| | 46% |
| 1-142 | 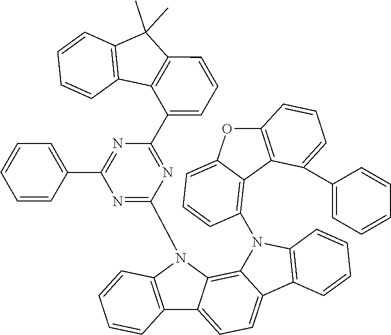 [SMILES: CC1(C)c2ccccc2-c2c(-c3nc(-c4ccccc4)nc(-n4c5ccccc5c5ccc6c7ccccc7n(-c7cccc8oc9cccc(-c%10ccccc%10)c9c78)c6c54)n3)cccc21] |
| | 50% |
| 1-143 | 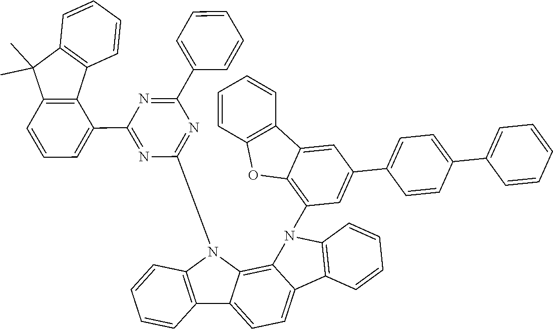 [SMILES: CC1(C)c2ccccc2-c2c(-c3nc(-c4ccccc4)nc(-n4c5ccccc5c5ccc6c7ccccc7n(-c7cc(-c8ccc(-c9ccccc9)cc8)cc8c7oc7ccccc78)c6c54)n3)cccc21] |
| | 51% |
| 1-144 | 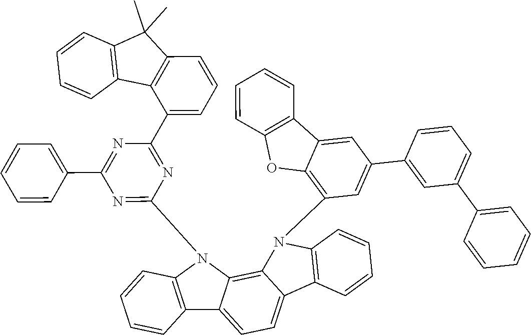 [SMILES: CC1(C)c2ccccc2-c2c(-c3nc(-c4ccccc4)nc(-n4c5ccccc5c5ccc6c7ccccc7n(-c7cc(-c8cccc(-c9ccccc9)c8)cc8c7oc7ccccc78)c6c54)n3)cccc21] |
| | 53% |
| 1-153 | 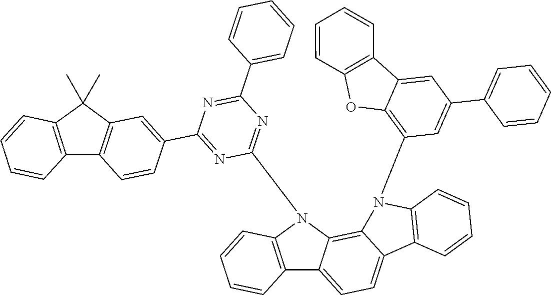 [SMILES: CC1(C)c2ccccc2-c2ccc(-c3nc(-c4ccccc4)nc(-n4c5ccccc5c5ccc6c7ccccc7n(-c7cc(-c8ccccc8)cc8c7oc7ccccc78)c6c54)n3)cc21] |
| | 48% |
| 1-331 | 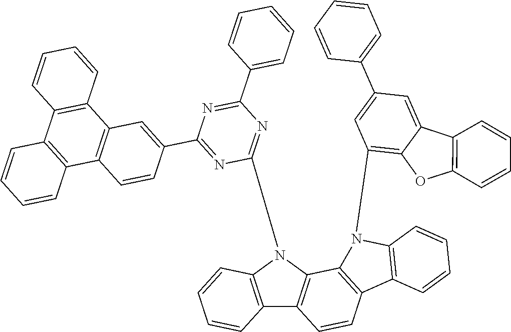 [SMILES: c1ccc(-c2cc(-n3c4ccccc4c4ccc5c6ccccc6n(-c6nc(-c7ccccc7)nc(-c7ccc8c9ccccc9c9ccccc9c8c7)n6)c5c43)c3oc4ccccc4c3c2)cc1] |
| | 51% |
| 1-332 | 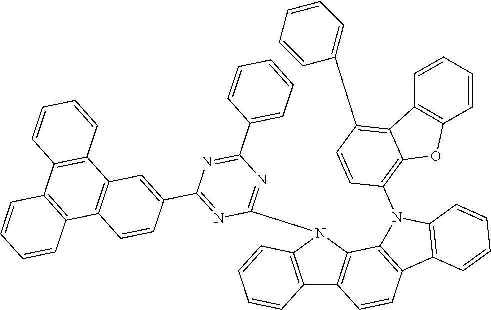 [SMILES: c1ccc(-c2nc(-c3ccc4c5ccccc5c5ccccc5c4c3)nc(-n3c4ccccc4c4ccc5c6ccccc6n(-c6ccc(-c7ccccc7)c7c6oc6ccccc67)c5c43)n2)cc1] |
| | 55% |
| 1-335 | 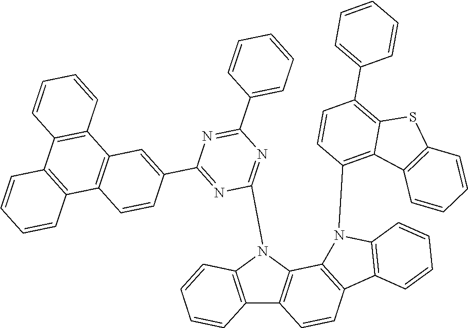 [SMILES: c1ccc(-c2nc(-c3ccc4c5ccccc5c5ccccc5c4c3)nc(-n3c4ccccc4c4ccc5c6ccccc6n(-c6ccc(-c7ccccc7)c7sc8ccccc8c67)c5c43)n2)cc1] |
| | 57% |
| 1-230 | 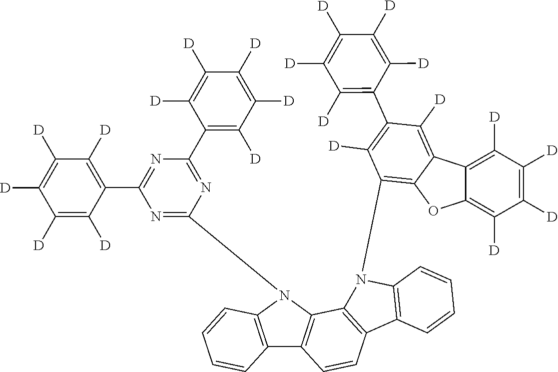 [SMILES: [2H]c1c([2H])c([2H])c(-c2nc(-c3c([2H])c([2H])c([2H])c([2H])c3[2H])nc(-n3c4ccccc4c4ccc5c6ccccc6n(-c6c([2H])c(-c7c([2H])c([2H])c([2H])c([2H])c7[2H])c([2H])c7c6oc6c([2H])c([2H])c([2H])c([2H])c67)c5c43)n2)c([2H])c1[2H]] |
| | 51% |
| 1-242 | 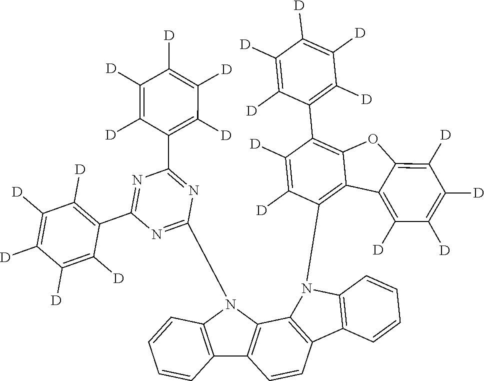 [SMILES: [2H]c1c([2H])c([2H])c(-c2nc(-c3c([2H])c([2H])c([2H])c([2H])c3[2H])nc(-n3c4ccccc4c4ccc5c6ccccc6n(-c6c([2H])c([2H])c(-c7c([2H])c([2H])c([2H])c([2H])c7[2H])c7oc8c([2H])c([2H])c([2H])c([2H])c8c67)c5c43)n2)c([2H])c1[2H]] |
| | 55% |
| 1-245 | 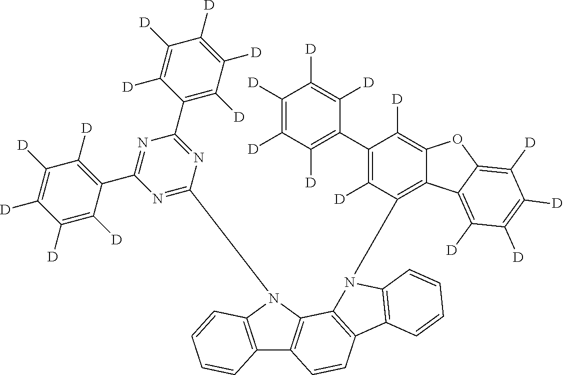 [SMILES: [2H]c1c([2H])c([2H])c(-c2nc(-c3c([2H])c([2H])c([2H])c([2H])c3[2H])nc(-n3c4ccccc4c4ccc5c6ccccc6n(-c6c([2H])c(-c7c([2H])c([2H])c([2H])c([2H])c7[2H])c([2H])c7oc8c([2H])c([2H])c([2H])c([2H])c8c67)c5c43)n2)c([2H])c1[2H]] |
| | 53% |
| 1-248 | 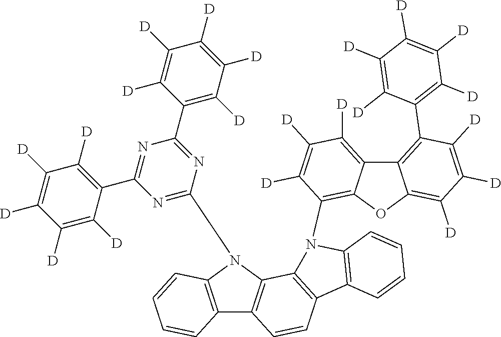 [SMILES: [2H]c1c([2H])c([2H])c(-c2nc(-c3c([2H])c([2H])c([2H])c([2H])c3[2H])nc(-n3c4ccccc4c4ccc5c6ccccc6n(-c6c([2H])c([2H])c([2H])c7c6oc6c([2H])c([2H])c([2H])c(-c8c([2H])c([2H])c([2H])c([2H])c8[2H])c67)c5c43)n2)c([2H])c1[2H]] |
| | 56% |
| 1-266 | 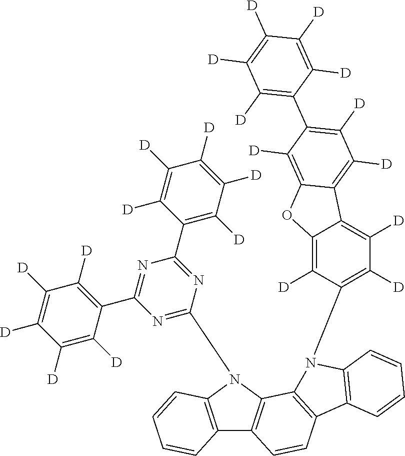 [SMILES: [2H]c1c([2H])c([2H])c(-c2nc(-c3c([2H])c([2H])c([2H])c([2H])c3[2H])nc(-n3c4ccccc4c4ccc5c6ccccc6n(-c6c([2H])c([2H])c7c(oc8c([2H])c(-c9c([2H])c([2H])c([2H])c([2H])c9[2H])c([2H])c([2H])c87)c6[2H])c5c43)n2)c([2H])c1[2H]] |
| | 52% |
| 1-269 | 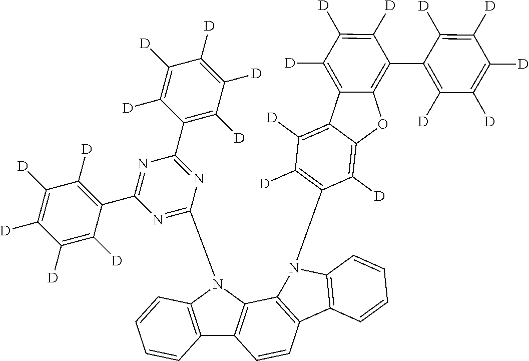 [SMILES: [2H]c1c([2H])c([2H])c(-c2nc(-c3c([2H])c([2H])c([2H])c([2H])c3[2H])nc(-n3c4ccccc4c4ccc5c6ccccc6n(-c6c([2H])c([2H])c7c(oc8c(-c9c([2H])c([2H])c([2H])c([2H])c9[2H])c([2H])c([2H])c([2H])c87)c6[2H])c5c43)n2)c([2H])c1[2H]] |
| | 52% |

### Preparation Example 2. Preparation of Compound 1-193

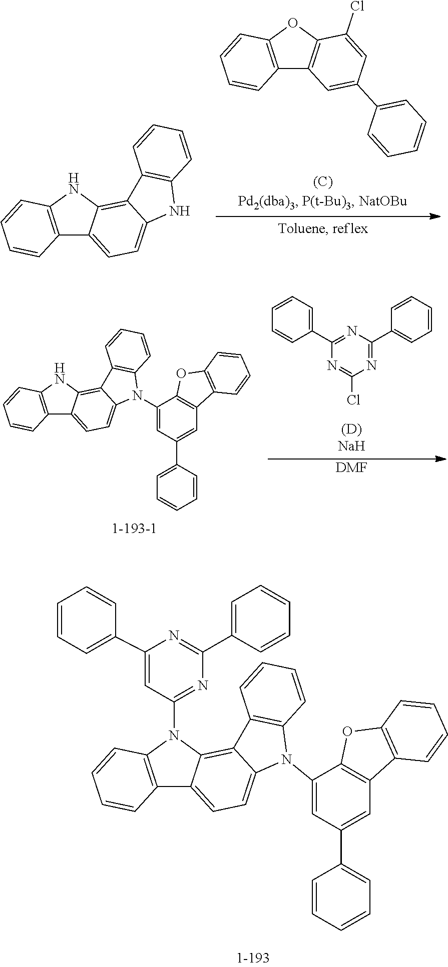

### Preparation Example 2-1. Preparation of Compound 1-193-1

4-Chloro-2-phenyldibenzo[b,d]furan (C) (25.1 g, 90 mM), 5,12-dihydroindolo[3,2-a]carbazole (25.6 g, 100 mM), tris(dibenzylideneacetone) dipalladium (Pd₂(dba)₃) (4.57 g, 5 mM), tri-tert-butylphosphine (P(t-Bu)₃) (2.02 g, 10 mM) and sodium tert-butoxide (NatOBu) (19.22 g, 200 mM) were dissolved in toluene (250 mL), and refluxed for 24 hours.

After the reaction was completed, the result was extracted by introducing distilled water and dichloromethane (DCM) thereto at room temperature, and after drying the organic layer with anhydrous magnesium sulfate (MgSO₄), the solvent was removed using a rotary evaporator. The reaction material was purified by column chromatography (dichloromethane:hexane=1:2), and recrystallized with methanol to obtain target Compound 1-193-1 (34.8 g).

### Preparation Example 2-2. Preparation of Compound 1-193

After dissolving Compound 1-193-1 (34 g, 68.2 mM) in dimethylformamide (DMF) (300 mL), sodium hydride (NaH) (3.27 g, 136.4 mM) was slowly introduced thereto. After 1 hour, 2-chloro-4,6-diphenyl-1,3,5-triazine (D) (23.7 g, 88.7 mM) was introduced thereto, and the mixture was reacted for 6 hours.

After the reaction was completed, the result was extracted by introducing distilled water and dichloromethane (DCM) thereto at room temperature, and after drying the organic layer with anhydrous magnesium sulfate (MgSO₄), the solvent was removed using a rotary evaporator. The reaction material was purified by column chromatography (dichloromethane:hexane=1:2), and recrystallized with methanol to obtain target Compound 1-193 (35.9 g, yield 72.1%).

Target compounds were synthesized as in the following Table 2 in the same manner as in Preparation Example 2 except that Compound C of the following Table 2 was used instead of 4-chloro-2-phenyldibenzo[b, d]furan (C), and Compound D of the following Table 2 was used instead of 2-chloro-4,6-diphenyl-1,3,5-triazine (D).

TABLE 2

| Com- | | | |
|---|---|---|---|
| pound | | | |
| No. | Compound C | Compound D | Target Compound (Yield%) |
| 1-194 | 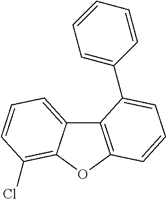 [SMILES: Clc1cccc2c1oc1cccc(-c3ccccc3)c12] | 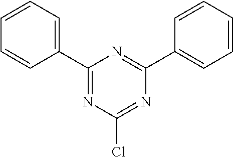 [SMILES: Clc1nc(-c2ccccc2)nc(-c2ccccc2)n1] | 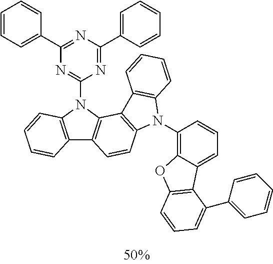 [SMILES: c1ccc(-c2nc(-c3ccccc3)nc(-n3c4ccccc4c4ccc5c(c6ccccc6n5-c5cccc6c5oc5cccc(-c7ccccc7)c56)c43)n2)cc1] |
| 1-316 | 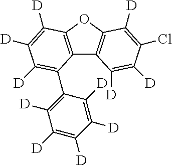 [SMILES: [2H]c1c([2H])c([2H])c(-c2c([2H])c([2H])c([2H])c3oc4c([2H])c(Cl)c([2H])c([2H])c4c23)c([2H])c1[2H]] | 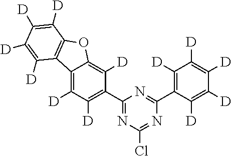 [SMILES: [2H]c1c([2H])c([2H])c(-c2nc(Cl)nc(-c3c([2H])c([2H])c4c(oc5c([2H])c([2H])c([2H])c([2H])c54)c3[2H])n2)c([2H])c1[2H]] | 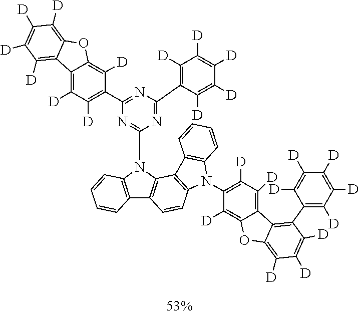 [SMILES: [2H]c1c([2H])c([2H])c(-c2nc(-c3c([2H])c([2H])c4c(oc5c([2H])c([2H])c([2H])c([2H])c54)c3[2H])nc(-n3c4ccccc4c4ccc5c(c6ccccc6n5-c5c([2H])c([2H])c6c(oc7c([2H])c([2H])c([2H])c(-c8c([2H])c([2H])c([2H])c([2H])c8[2H])c76)c5[2H])c43)n2)c([2H])c1[2H]] |

### Preparation Example 3. Preparation of Compound 1-205

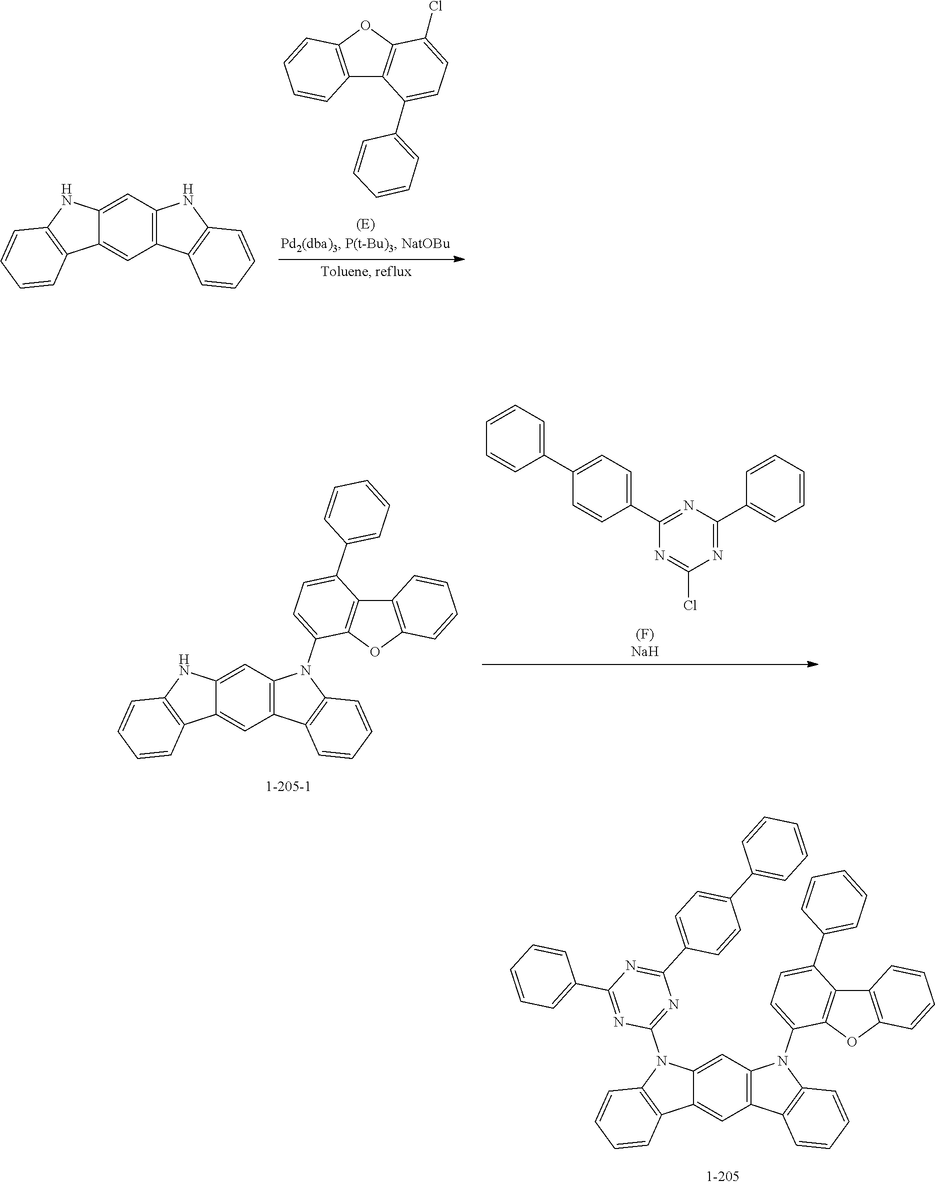 [SMILES: c1ccc2c(c1)[nH]c1cc3[nH]c4ccccc4c3cc12 ; Clc1ccc(-c2ccccc2)c2c1oc1ccccc12 ; c1ccc(-c2ccc(-n3c4ccccc4c4cc5c(cc43)[nH]c3ccccc35)c3oc4ccccc4c23)cc1 ; Clc1nc(-c2ccccc2)nc(-c2ccc(-c3ccccc3)cc2)n1 ; c1ccc(-c2ccc(-c3nc(-c4ccccc4)nc(-n4c5ccccc5c5cc6c7ccccc7n(-c7ccc(-c8ccccc8)c8c7oc7ccccc78)c6cc54)n3)cc2)cc1]

### Preparation Example 3-1. Preparation of Compound 1-205-1

4-Chloro-1-phenyldibenzo[b,d]furan (E) (12.6 g, 50 mM), 11,12-dihydroindolo[3,2-a]carbazole (12.8 g, 50 mM), tris(dibenzylideneacetone) dipalladium (Pd₂(dba)₃) (2.28 g, 2.5 mM), tri-tert-butylphosphine (P(t-Bu)₃) (1.01 g, 5 mM) and sodium tert-butoxide (NatOBu) (9.61 g, 100 mM) were dissolved in toluene (130 mL), and refluxed for 24 hours.

After the reaction was completed, the result was extracted by introducing distilled water and dichloromethane (DCM) thereto at room temperature, and after drying the organic layer with anhydrous magnesium sulfate (MgSO₄), the solvent was removed using a rotary evaporator. The reaction material was purified by column chromatography (dichloromethane:hexane=1:2), and recrystallized with methanol to obtain target Compound 1-205-1 (15.4 g).

### Preparation Example 3-2. Preparation of Compound 1-205

After dissolving Compound 1-205-1 (15 g, 30 mM) in dimethylformamide (DMF) (150 mL), sodium hydride (NaH) (1.44 g, 60 mM) was slowly introduced thereto. After 1 hour, 2-([1,1′-diphenyl]-5-yl)-4-chloro-6-phenyl-1,3,5-triazine (F) (13.4 g, 39 mM) was introduced thereto, and the mixture was reacted for 6 hours.

After the reaction was completed, the result was extracted by introducing distilled water and dichloromethane (DCM) thereto at room temperature, and after drying the organic layer with anhydrous magnesium sulfate (MgSO₄), the solvent was removed using a rotary evaporator. The reaction material was purified by column chromatography (dichloromethane:hexane=1:2), and recrystallized with methanol to obtain target Compound 1-205 (21.7 g, yield 86%).

Target compounds were synthesized as in the following Table 3 in the same manner as in Preparation Example 3 except that Compound E of the following Table 3 was used instead of 4-chloro-1-phenyldibenzo[b, d]furan (E), and Compound F of the following Table 3 was used instead of 2-([1,1′-diphenyl]-5-yl)-4-chloro-6-phenyl-1,3,5-triazine (F).

TABLE 3

| Compound | | | Target Compound |
|---|---|---|---|
| No. | Compound E | Compound F | (Yield %) |
| 1-206 | 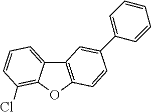 [SMILES: Clc1cccc2c1oc1ccc(-c3ccccc3)cc12] | 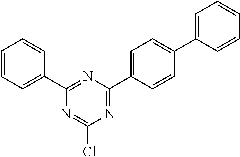 [SMILES: Clc1nc(-c2ccccc2)nc(-c2ccc(-c3ccccc3)cc2)n1] | 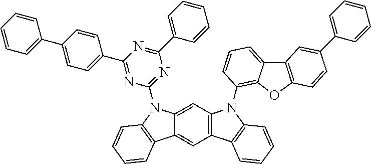 [SMILES: c1ccc(-c2ccc(-c3nc(-c4ccccc4)nc(-n4c5ccccc5c5cc6c7ccccc7n(-c7cccc8c7oc7ccc(-c9ccccc9)cc78)c6cc54)n3)cc2)cc1] |
| | | | 54% |
| 1-207 | 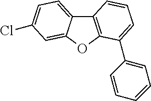 [SMILES: Clc1ccc2c(c1)oc1c(-c3ccccc3)cccc12] | 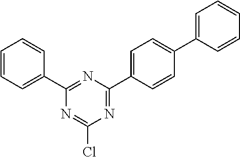 [SMILES: Clc1nc(-c2ccccc2)nc(-c2ccc(-c3ccccc3)cc2)n1] | 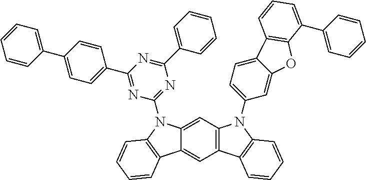 [SMILES: c1ccc(-c2ccc(-c3nc(-c4ccccc4)nc(-n4c5ccccc5c5cc6c7ccccc7n(-c7ccc8c(c7)oc7c(-c9ccccc9)cccc78)c6cc54)n3)cc2)cc1] |
| | | | 51% |
| 1-208 | 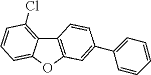 [SMILES: Clc1cccc2oc3cc(-c4ccccc4)ccc3c12] | 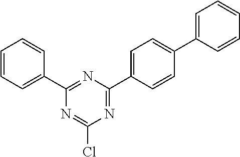 [SMILES: Clc1nc(-c2ccccc2)nc(-c2ccc(-c3ccccc3)cc2)n1] | 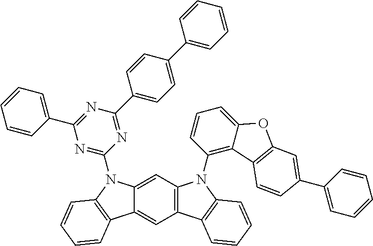 [SMILES: c1ccc(-c2ccc(-c3nc(-c4ccccc4)nc(-n4c5ccccc5c5cc6c7ccccc7n(-c7cccc8oc9cc(-c%10ccccc%10)ccc9c78)c6cc54)n3)cc2)cc1] |
| | | | 53% |
| 1-319 | 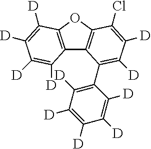 [SMILES: [2H]c1c([2H])c([2H])c(-c2c([2H])c([2H])c(Cl)c3oc4c([2H])c([2H])c([2H])c([2H])c4c23)c([2H])c1[2H]] | 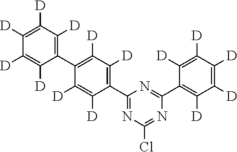 [SMILES: [2H]c1c([2H])c([2H])c(-c2nc(Cl)nc(-c3c([2H])c([2H])c(-c4c([2H])c([2H])c([2H])c([2H])c4[2H])c([2H])c3[2H])n2)c([2H])c1[2H]] | 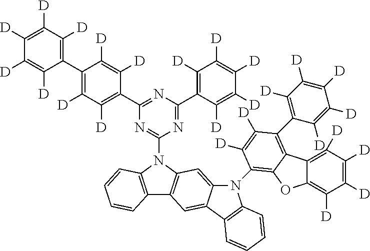 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-n3c4ccccc4c4cc5c6ccccc6n(-c6c([2H])c([2H])c(-c7c([2H])c([2H])c([2H])c([2H])c7[2H])c7c6oc6c([2H])c([2H])c([2H])c([2H])c67)c5cc43)n2)c([2H])c1[2H]] |
| | | | 55% |
| 1-322 | 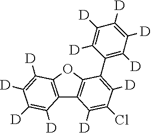 [SMILES: [2H]c1c([2H])c([2H])c(-c2c([2H])c(Cl)c([2H])c3c2oc2c([2H])c([2H])c([2H])c([2H])c23)c([2H])c1[2H]] | 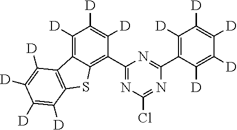 [SMILES: [2H]c1c([2H])c([2H])c(-c2nc(Cl)nc(-c3c([2H])c([2H])c([2H])c4c3sc3c([2H])c([2H])c([2H])c([2H])c34)n2)c([2H])c1[2H]] | 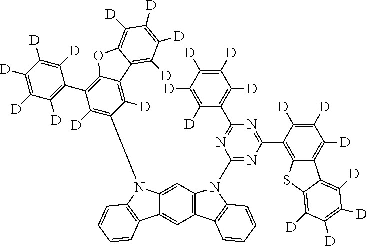 [SMILES: [2H]c1c([2H])c([2H])c(-c2nc(-c3c([2H])c([2H])c([2H])c4c3sc3c([2H])c([2H])c([2H])c([2H])c34)nc(-n3c4ccccc4c4cc5c6ccccc6n(-c6c([2H])c(-c7c([2H])c([2H])c([2H])c([2H])c7[2H])c7oc8c([2H])c([2H])c([2H])c([2H])c8c7c6[2H])c5cc43)n2)c([2H])c1[2H]] |
| | | | 51% |

### Preparation Example 4. Preparation of Compound 1-221

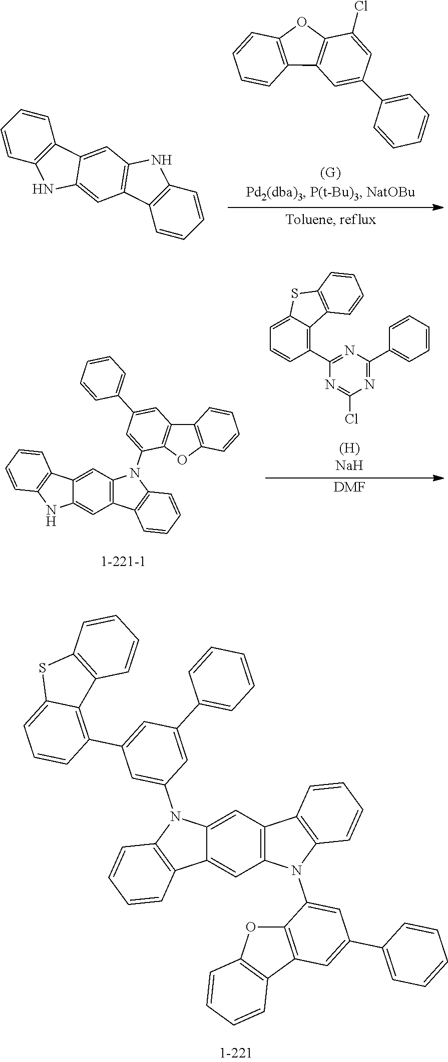

### Preparation Example 4-1. Preparation of Compound 1-221-1

4-Chloro-2-phenyldibenzo[b,d]furan (G) (12.6 g, 50 mM), 5,12-dihydroindolo[3,2-a]carbazole (12.8 g, 50 mM), tris(dibenzylideneacetone) dipalladium (Pd₂(dba)₃) (2.28 g, 2.5 mM), tri-tert-butylphosphine (P(t-Bu)₃) (1.01 g, 5 mM) and sodium tert-butoxide (NatOBu) (9.61 g, 100 mM) were dissolved in toluene (130 mL), and refluxed for 24 hours.

After the reaction was completed, the result was extracted by introducing distilled water and dichloromethane (DCM) thereto at room temperature, and after drying the organic layer with anhydrous magnesium sulfate (MgSO₄), the solvent was removed using a rotary evaporator. The reaction material was purified by column chromatography (dichloromethane:hexane=1:2), and recrystallized with methanol to obtain target Compound 1-221-1 (15.4 g).

### Preparation Example 4-2. Preparation of Compound 1-221

After dissolving Compound 1-221-1 (15 g, 30 mM) in dimethylformamide (DMF) (150 mL), sodium hydride (NaH) (1.44 g, 60 mM) was slowly introduced thereto. After 1 hour, 2-chloro-4-(dibenzo[b,d]thiophen-1-yl)-6-phenyl-1,3,5-triazine (H) (13.4 g, 39 mM) was introduced thereto, and the mixture was reacted for 6 hours.

After the reaction was completed, the result was extracted by introducing distilled water and dichloromethane (DCM) thereto at room temperature, and after drying the organic layer with anhydrous magnesium sulfate (MgSO₄), the solvent was removed using a rotary evaporator. The reaction material was purified by column chromatography (dichloromethane:hexane=1:2), and recrystallized with methanol to obtain target Compound 1-221 (21.7 g, yield 86%).

Target compounds were synthesized as in the following Table 4 in the same manner as in Preparation Example 4 except that Compound G of the following Table 4 was used instead of 4-chloro-2-phenyldibenzo[b,d]furan (G), and Compound H of the following Table 4 was used instead of 2-chloro-4-(dibenzo[b,d]thiophen-1-yl)-6-phenyl-1,3,5-triazine (H).

TABLE 4

| Compound | | | Target Compound |
|---|---|---|---|
| No. | Compound G | Compound H | (Yield %) |
| 1-222 | 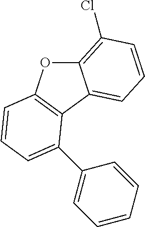 [SMILES: Clc1cccc2c1oc1cccc(-c3ccccc3)c12] | 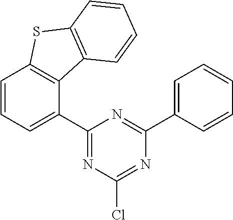 [SMILES: Clc1nc(-c2ccccc2)nc(-c2cccc3sc4ccccc4c23)n1] | 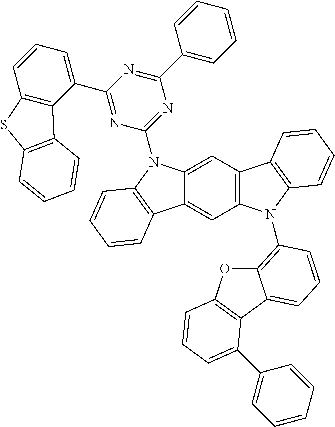 [SMILES: c1ccc(-c2nc(-c3cccc4sc5ccccc5c34)nc(-n3c4ccccc4c4cc5c(cc43)c3ccccc3n5-c3cccc4c3oc3cccc(-c5ccccc5)c34)n2)cc1] |
| | | | 57% |
| 1-223 | 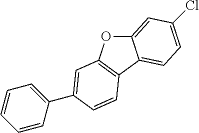 [SMILES: Clc1ccc2c(c1)oc1cc(-c3ccccc3)ccc12] | 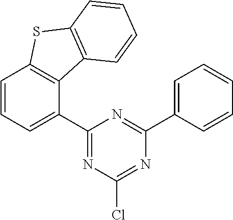 [SMILES: Clc1nc(-c2ccccc2)nc(-c2cccc3sc4ccccc4c23)n1] | 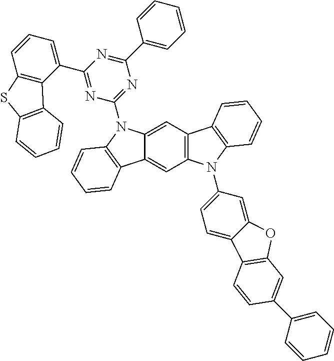 [SMILES: c1ccc(-c2ccc3c(c2)oc2cc(-n4c5ccccc5c5cc6c(cc54)c4ccccc4n6-c4nc(-c5ccccc5)nc(-c5cccc6sc7ccccc7c56)n4)ccc23)cc1] |
| | | | 55% |
| 1-224 | 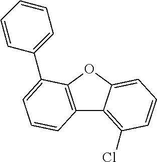 [SMILES: Clc1cccc2oc3c(-c4ccccc4)cccc3c12] | 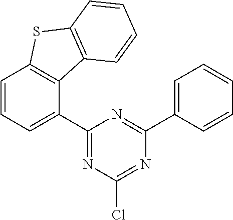 [SMILES: Clc1nc(-c2ccccc2)nc(-c2cccc3sc4ccccc4c23)n1] | 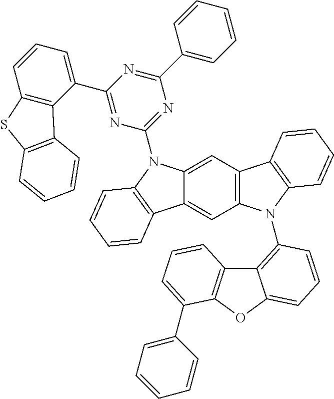 [SMILES: c1ccc(-c2nc(-c3cccc4sc5ccccc5c34)nc(-n3c4ccccc4c4cc5c(cc43)c3ccccc3n5-c3cccc4oc5c(-c6ccccc6)cccc5c34)n2)cc1] |
| | | | 54% |
| 1-325 | 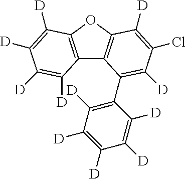 [SMILES: [2H]c1c([2H])c([2H])c(-c2c([2H])c(Cl)c([2H])c3oc4c([2H])c([2H])c([2H])c([2H])c4c23)c([2H])c1[2H]] | 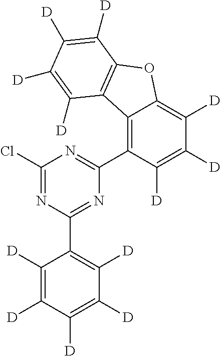 [SMILES: [2H]c1c([2H])c([2H])c(-c2nc(Cl)nc(-c3c([2H])c([2H])c([2H])c4oc5c([2H])c([2H])c([2H])c([2H])c5c34)n2)c([2H])c1[2H]] | 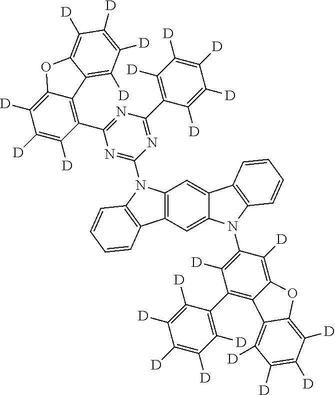 [SMILES: [2H]c1c([2H])c([2H])c(-c2nc(-c3c([2H])c([2H])c([2H])c4oc5c([2H])c([2H])c([2H])c([2H])c5c34)nc(-n3c4ccccc4c4cc5c(cc43)c3ccccc3n5-c3c([2H])c(-c4c([2H])c([2H])c([2H])c([2H])c4[2H])c4c(oc5c([2H])c([2H])c([2H])c([2H])c54)c3[2H])n2)c([2H])c1[2H]] |
| | | | 52% |

### Preparation Example 5. Preparation of Compound 1-229

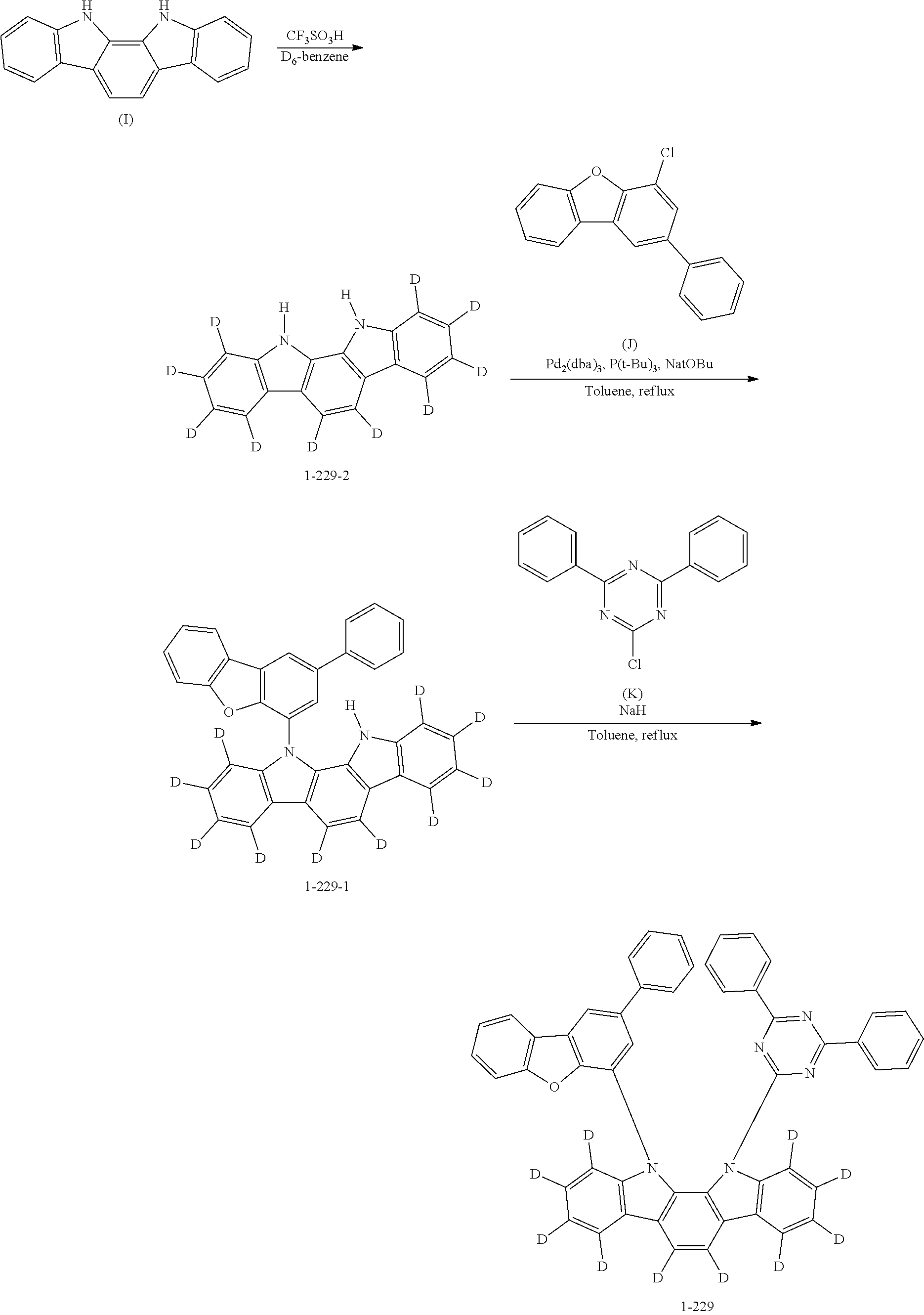

### Preparation Example 5-1. Preparation of Compound 1-229-2

After introducing 11,12-dihydroindolo[2,3-a]carbazole (I) (10 g, 39.0 mmol) to D₆-benzene (1000 mL), triflic acid (CF₃SO₃H) (170 g, 1075 mmol) was introduced thereto, and the mixture was stirred at 50° C.

When the reaction was completed, the result was neutralized with D₂O and then extracted by introducing an aqueous sodium carbonate (Na₂CO₃) solution and dichloromethane (DCM) thereto at room temperature, and after drying the organic layer with anhydrous magnesium sulfate (MgSO₄), the solvent was removed using a rotary evaporator. The reaction material was purified by column chromatography (dichloromethane:hexane=1:2), and recrystallized with methanol to obtain target Compound 1-229-2 (9.9 g, yield 95%).

### Preparation Example 5-2. Preparation of Compound 1-229-1

4-Chloro-2-phenyldibenzo[b,d]furan (J) (12.6 g, 50 mM), 11,12-dihydroindolo[3,2-a]carbazole-1,2,3,4,5,6,7,8,9,10-d10 (9.5 g, 36 mM), tris(dibenzylideneacetone)dipalladium (Pd₂(dba)₃) (1.65 g, 1.8 mM), tri-tert-butylphosphine (P(t-Bu)₃) (0.73 g, 3.6 mM) and sodium tert-butoxide (NatOBu) (5.96 g, 62 mM) were dissolved in toluene (120 mL), and refluxed for 24 hours.

After the reaction was completed, the result was extracted by introducing distilled water and dichloromethane (DCM) thereto at room temperature, and after drying the organic layer with anhydrous magnesium sulfate (MgSO₄), the solvent was removed using a rotary evaporator. The reaction material was purified by column chromatography (dichloromethane:hexane=1:2), and recrystallized with methanol to obtain target Compound 1-229-1 (11.1 g, yield 61%).

### Preparation Example 5-3. Preparation of Compound 1-229

After dissolving Compound 1-229-1 (11 g, 21.6 mM) in dimethylformamide (DMF) (100 mL), sodium hydride (NaH) (1.04 g, 43.2 mM) was slowly introduced thereto. After 1 hour, 2-chloro-4,6-diphenyl-1,3,5-triazine (K) (7.52 g, 28.1 mM) was introduced thereto, and the mixture was reacted for 6 hours.

After the reaction was completed, the result was extracted by introducing distilled water and dichloromethane (DCM) thereto at room temperature, and after drying the organic layer with anhydrous magnesium sulfate (MgSO₄), the solvent was removed using a rotary evaporator. The reaction material was purified by column chromatography (dichloromethane:hexane=1:2), and recrystallized with methanol to obtain target Compound 1-229 (12.4 g, yield 87%).

Target compounds were synthesized as in the following Table 5 in the same manner as in Preparation Example 5 except that Compound I of the following Table 5 was used instead of 11,12-dihydroindolo[2,3-a]carbazole (I), Compound J of the following Table 5 was used instead of 4-chloro-2-phenyldibenzo[b,d]furan (J), and Compound K of the following Table 5 was used instead of 2-chloro-4,6-diphenyl-1,3,5-triazine (K).

TABLE 5

| Compound | | | | Target Compound |
|---|---|---|---|---|
| No. | Compound I | Compound J | Compound K | (Yields) |
| 1-229 | 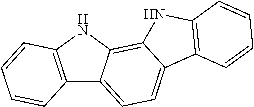 [SMILES: c1ccc2c(c1)[nH]c1c2ccc2c3ccccc3[nH]c21] | 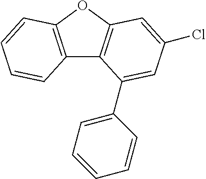 [SMILES: Clc1cc(-c2ccccc2)c2c(c1)oc1ccccc12] | 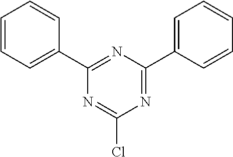 [SMILES: Clc1nc(-c2ccccc2)nc(-c2ccccc2)n1] | 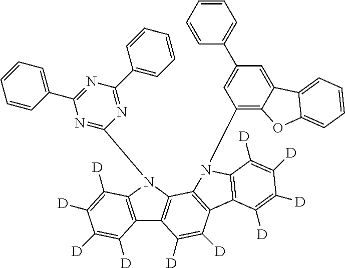 [SMILES: [2H]c1c([2H])c([2H])c2c(c1[2H])c1c([2H])c([2H])c3c4c([2H])c([2H])c([2H])c([2H])c4n(-c4cc(-c5ccccc5)cc5c4oc4ccccc45)c3c1n2-c1nc(-c2ccccc2)nc(-c2ccccc2)n1] |
| | | | | 54% |
| 1-231 | 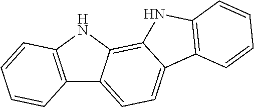 [SMILES: c1ccc2c(c1)[nH]c1c2ccc2c3ccccc3[nH]c21] | 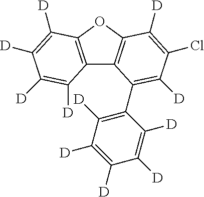 [SMILES: [2H]c1c([2H])c([2H])c(-c2c([2H])c(Cl)c([2H])c3oc4c([2H])c([2H])c([2H])c([2H])c4c23)c([2H])c1[2H]] | 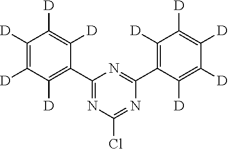 [SMILES: [2H]c1c([2H])c([2H])c(-c2nc(Cl)nc(-c3c([2H])c([2H])c([2H])c([2H])c3[2H])n2)c([2H])c1[2H]] | 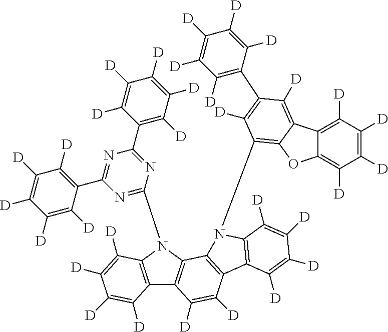 [SMILES: [2H]c1c([2H])c([2H])c(-c2nc(-c3c([2H])c([2H])c([2H])c([2H])c3[2H])nc(-n3c4c([2H])c([2H])c([2H])c([2H])c4c4c([2H])c([2H])c5c6c([2H])c([2H])c([2H])c([2H])c6n(-c6c([2H])c(-c7c([2H])c([2H])c([2H])c([2H])c7[2H])c([2H])c7c6oc6c([2H])c([2H])c([2H])c([2H])c67)c5c43)n2)c([2H])c1[2H]] |
| | | | | 51% |
| 1-241 | 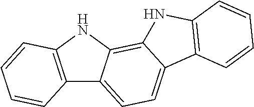 [SMILES: c1ccc2c(c1)[nH]c1c2ccc2c3ccccc3[nH]c21] | 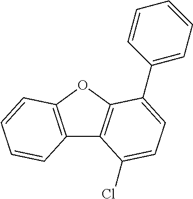 [SMILES: Clc1ccc(-c2ccccc2)c2oc3ccccc3c12] | 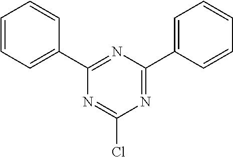 [SMILES: Clc1nc(-c2ccccc2)nc(-c2ccccc2)n1] | 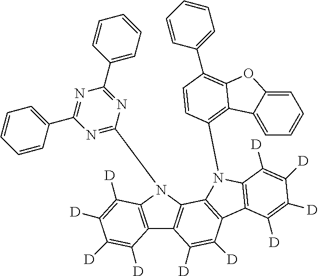 [SMILES: [2H]c1c([2H])c([2H])c2c(c1[2H])c1c([2H])c([2H])c3c4c([2H])c([2H])c([2H])c([2H])c4n(-c4ccc(-c5ccccc5)c5oc6ccccc6c45)c3c1n2-c1nc(-c2ccccc2)nc(-c2ccccc2)n1] |
| | | | | 53% |
| 1-243 | 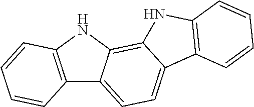 [SMILES: c1ccc2c(c1)[nH]c1c2ccc2c3ccccc3[nH]c21] | 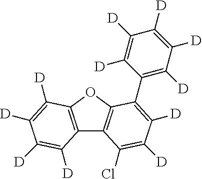 [SMILES: [2H]c1c([2H])c([2H])c(-c2c([2H])c([2H])c(Cl)c3c2oc2c([2H])c([2H])c([2H])c([2H])c23)c([2H])c1[2H]] | 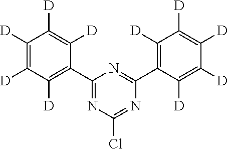 [SMILES: [2H]c1c([2H])c([2H])c(-c2nc(Cl)nc(-c3c([2H])c([2H])c([2H])c([2H])c3[2H])n2)c([2H])c1[2H]] | 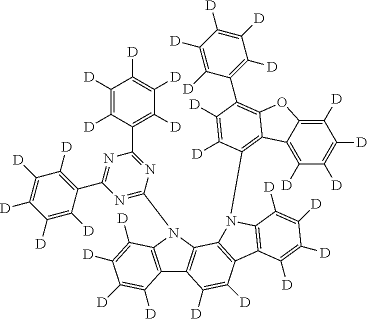 [SMILES: [2H]c1c([2H])c([2H])c(-c2nc(-c3c([2H])c([2H])c([2H])c([2H])c3[2H])nc(-n3c4c([2H])c([2H])c([2H])c([2H])c4c4c([2H])c([2H])c5c6c([2H])c([2H])c([2H])c([2H])c6n(-c6c([2H])c([2H])c(-c7c([2H])c([2H])c([2H])c([2H])c7[2H])c7oc8c([2H])c([2H])c([2H])c([2H])c8c67)c5c43)n2)c([2H])c1[2H]] |
| | | | | 55% |
| 1-244 | 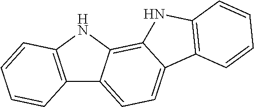 [SMILES: c1ccc2c(c1)[nH]c1c2ccc2c3ccccc3[nH]c21] | 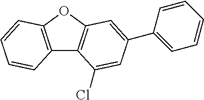 [SMILES: Clc1cc(-c2ccccc2)cc2oc3ccccc3c12] | 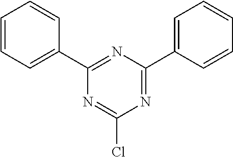 [SMILES: Clc1nc(-c2ccccc2)nc(-c2ccccc2)n1] | 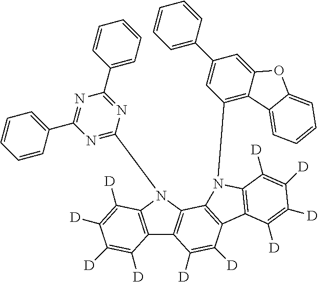 [SMILES: [2H]c1c([2H])c([2H])c2c(c1[2H])c1c([2H])c([2H])c3c4c([2H])c([2H])c([2H])c([2H])c4n(-c4cc(-c5ccccc5)cc5oc6ccccc6c45)c3c1n2-c1nc(-c2ccccc2)nc(-c2ccccc2)n1] |
| | | | | 54% |
| 1-246 | 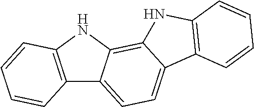 [SMILES: c1ccc2c(c1)[nH]c1c2ccc2c3ccccc3[nH]c21] | 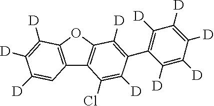 [SMILES: [2H]c1cc2c(oc3c([2H])c(-c4c([2H])c([2H])c([2H])c([2H])c4[2H])c([2H])c(Cl)c32)c([2H])c1[2H]] | 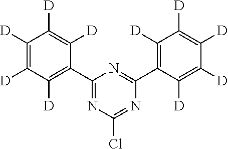 [SMILES: [2H]c1c([2H])c([2H])c(-c2nc(Cl)nc(-c3c([2H])c([2H])c([2H])c([2H])c3[2H])n2)c([2H])c1[2H]] | 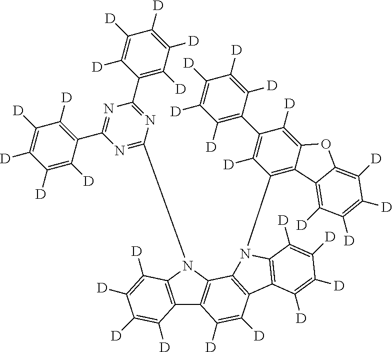 [SMILES: [2H]c1c([2H])c([2H])c(-c2nc(-c3c([2H])c([2H])c([2H])c([2H])c3[2H])nc(-n3c4c([2H])c([2H])c([2H])c([2H])c4c4c([2H])c([2H])c5c6c([2H])c([2H])c([2H])c([2H])c6n(-c6c([2H])c(-c7c([2H])c([2H])c([2H])c([2H])c7[2H])c([2H])c7oc8c([2H])c([2H])c([2H])c([2H])c8c67)c5c43)n2)c([2H])c1[2H]] |
| | | | | 52% |
| 1-247 | 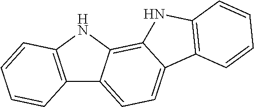 [SMILES: c1ccc2c(c1)[nH]c1c2ccc2c3ccccc3[nH]c21] | 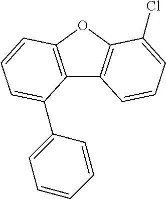 [SMILES: Clc1cccc2c1oc1cccc(-c3ccccc3)c12] | 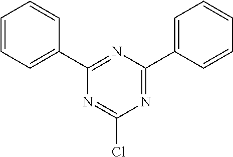 [SMILES: Clc1nc(-c2ccccc2)nc(-c2ccccc2)n1] | 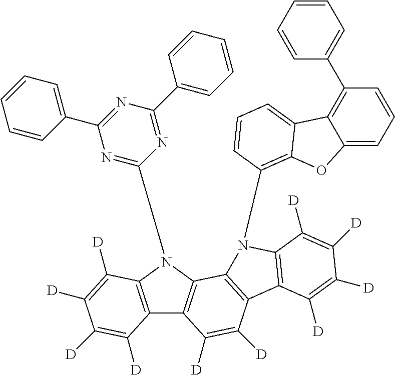 [SMILES: [2H]c1c([2H])c([2H])c2c(c1[2H])c1c([2H])c([2H])c3c4c([2H])c([2H])c([2H])c([2H])c4n(-c4cccc5c4oc4cccc(-c6ccccc6)c45)c3c1n2-c1nc(-c2ccccc2)nc(-c2ccccc2)n1] |
| | | | | 51% |
| 1-249 | 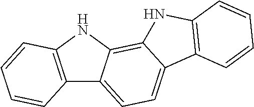 [SMILES: c1ccc2c(c1)[nH]c1c2ccc2c3ccccc3[nH]c21] | 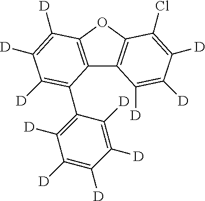 [SMILES: [2H]c1c([2H])c([2H])c(-c2c([2H])c([2H])c([2H])c3oc4c(Cl)c([2H])c([2H])c([2H])c4c23)c([2H])c1[2H]] | 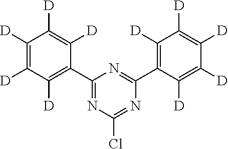 [SMILES: [2H]c1c([2H])c([2H])c(-c2nc(Cl)nc(-c3c([2H])c([2H])c([2H])c([2H])c3[2H])n2)c([2H])c1[2H]] | 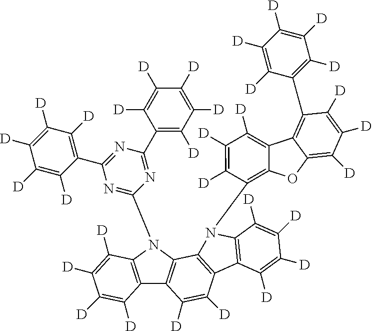 [SMILES: [2H]c1c([2H])c([2H])c(-c2nc(-c3c([2H])c([2H])c([2H])c([2H])c3[2H])nc(-n3c4c([2H])c([2H])c([2H])c([2H])c4c4c([2H])c([2H])c5c6c([2H])c([2H])c([2H])c([2H])c6n(-c6c([2H])c([2H])c([2H])c7c6oc6c([2H])c([2H])c([2H])c(-c8c([2H])c([2H])c([2H])c([2H])c8[2H])c67)c5c43)n2)c([2H])c1[2H]] |
| | | | | 49% |
| 1-265 | 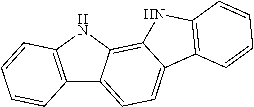 [SMILES: c1ccc2c(c1)[nH]c1c2ccc2c3ccccc3[nH]c21] | 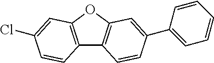 [SMILES: Clc1ccc2c(c1)oc1cc(-c3ccccc3)ccc12] | 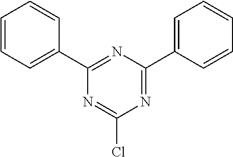 [SMILES: Clc1nc(-c2ccccc2)nc(-c2ccccc2)n1] | 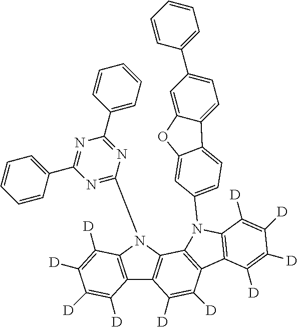 [SMILES: [2H]c1c([2H])c([2H])c2c(c1[2H])c1c([2H])c([2H])c3c4c([2H])c([2H])c([2H])c([2H])c4n(-c4nc(-c5ccccc5)nc(-c5ccccc5)n4)c3c1n2-c1ccc2c(c1)oc1cc(-c3ccccc3)ccc12] |
| | | | | 51% |
| 1-267 | 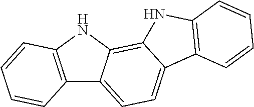 [SMILES: c1ccc2c(c1)[nH]c1c2ccc2c3ccccc3[nH]c21] | 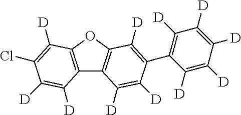 [SMILES: [2H]c1c([2H])c([2H])c(-c2c([2H])c([2H])c3c(oc4c([2H])c(Cl)c([2H])c([2H])c43)c2[2H])c([2H])c1[2H]] | 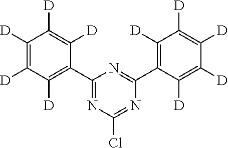 [SMILES: [2H]c1c([2H])c([2H])c(-c2nc(Cl)nc(-c3c([2H])c([2H])c([2H])c([2H])c3[2H])n2)c([2H])c1[2H]] | 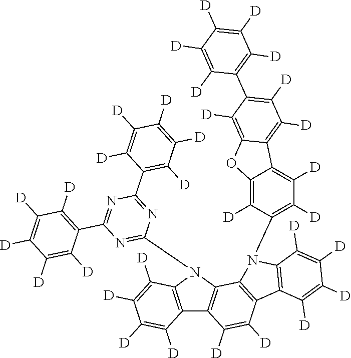 [SMILES: [2H]c1c([2H])c([2H])c(-c2nc(-c3c([2H])c([2H])c([2H])c([2H])c3[2H])nc(-n3c4c([2H])c([2H])c([2H])c([2H])c4c4c([2H])c([2H])c5c6c([2H])c([2H])c([2H])c([2H])c6n(-c6c([2H])c([2H])c7c(oc8c([2H])c(-c9c([2H])c([2H])c([2H])c([2H])c9[2H])c([2H])c([2H])c87)c6[2H])c5c43)n2)c([2H])c1[2H]] |
| | | | | 52% |
| 1-268 | 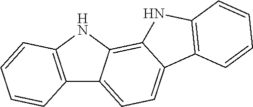 [SMILES: c1ccc2c(c1)[nH]c1c2ccc2c3ccccc3[nH]c21] | 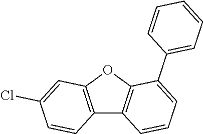 [SMILES: Clc1ccc2c(c1)oc1c(-c3ccccc3)cccc12] | 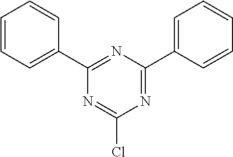 [SMILES: Clc1nc(-c2ccccc2)nc(-c2ccccc2)n1] | 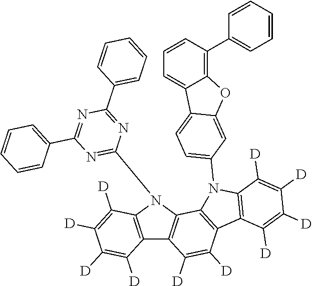 [SMILES: [2H]c1c([2H])c([2H])c2c(c1[2H])c1c([2H])c([2H])c3c4c([2H])c([2H])c([2H])c([2H])c4n(-c4nc(-c5ccccc5)nc(-c5ccccc5)n4)c3c1n2-c1ccc2c(c1)oc1c(-c3ccccc3)cccc12] |
| | | | | 55% |
| 1-270 | 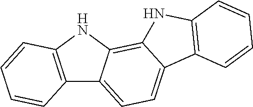 [SMILES: c1ccc2c(c1)[nH]c1c2ccc2c3ccccc3[nH]c21] | 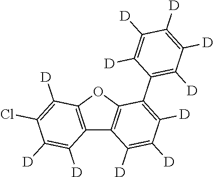 [SMILES: [2H]c1c([2H])c([2H])c(-c2c([2H])c([2H])c([2H])c3c2oc2c([2H])c(Cl)c([2H])c([2H])c23)c([2H])c1[2H]] | 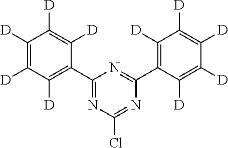 [SMILES: [2H]c1c([2H])c([2H])c(-c2nc(Cl)nc(-c3c([2H])c([2H])c([2H])c([2H])c3[2H])n2)c([2H])c1[2H]] | 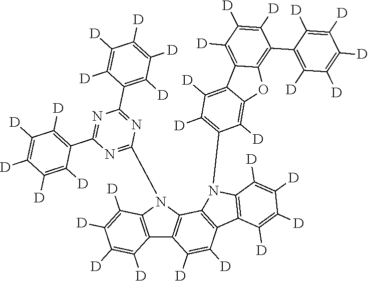 [SMILES: [2H]c1c([2H])c([2H])c(-c2nc(-c3c([2H])c([2H])c([2H])c([2H])c3[2H])nc(-n3c4c([2H])c([2H])c([2H])c([2H])c4c4c([2H])c([2H])c5c6c([2H])c([2H])c([2H])c([2H])c6n(-c6c([2H])c([2H])c7c(oc8c(-c9c([2H])c([2H])c([2H])c([2H])c9[2H])c([2H])c([2H])c([2H])c87)c6[2H])c5c43)n2)c([2H])c1[2H]] |
| | | | | 53% |
| 1-317 | 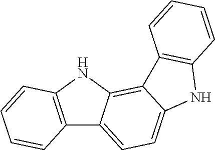 [SMILES: c1ccc2c(c1)[nH]c1c2ccc2[nH]c3ccccc3c21] | 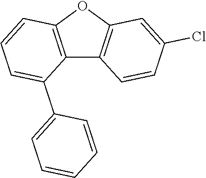 [SMILES: Clc1ccc2c(c1)oc1cccc(-c3ccccc3)c12] | 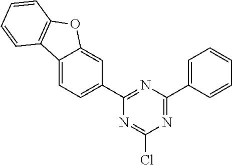 [SMILES: Clc1nc(-c2ccccc2)nc(-c2ccc3c(c2)oc2ccccc23)n1] | 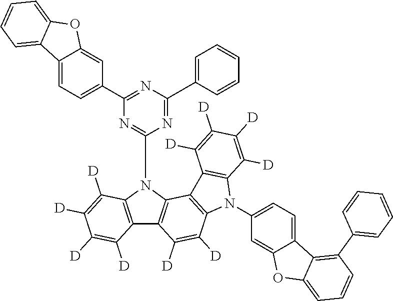 [SMILES: [2H]c1c([2H])c([2H])c2c(c1[2H])c1c(c([2H])c([2H])c3c4c([2H])c([2H])c([2H])c([2H])c4n(-c4nc(-c5ccccc5)nc(-c5ccc6c(c5)oc5ccccc56)n4)c31)n2-c1ccc2c(c1)oc1cccc(-c3ccccc3)c12] |
| | | | | 52% |
| 1-318 | 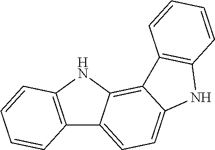 [SMILES: c1ccc2c(c1)[nH]c1c2ccc2[nH]c3ccccc3c21] | 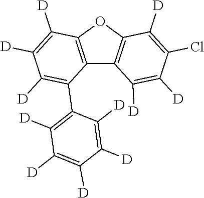 [SMILES: [2H]c1c([2H])c([2H])c(-c2c([2H])c([2H])c([2H])c3oc4c([2H])c(Cl)c([2H])c([2H])c4c23)c([2H])c1[2H]] | 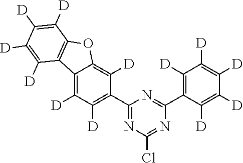 [SMILES: [2H]c1c([2H])c([2H])c(-c2nc(Cl)nc(-c3c([2H])c([2H])c4c(oc5c([2H])c([2H])c([2H])c([2H])c54)c3[2H])n2)c([2H])c1[2H]] | 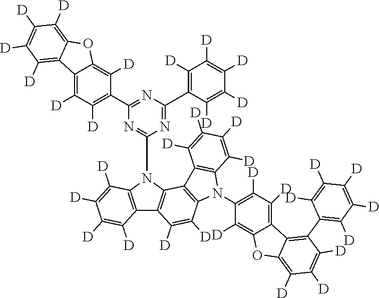 [SMILES: [2H]c1c([2H])c([2H])c(-c2nc(-c3c([2H])c([2H])c4c(oc5c([2H])c([2H])c([2H])c([2H])c54)c3[2H])nc(-n3c4c([2H])c([2H])c([2H])c([2H])c4c4c([2H])c([2H])c5c(c6c([2H])c([2H])c([2H])c([2H])c6n5-c5c([2H])c([2H])c6c(oc7c([2H])c([2H])c([2H])c(-c8c([2H])c([2H])c([2H])c([2H])c8[2H])c76)c5[2H])c43)n2)c([2H])c1[2H]] |
| | | | | 56% |
| 1-320 | 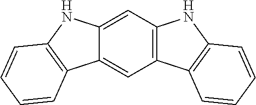 [SMILES: c1ccc2c(c1)[nH]c1cc3[nH]c4ccccc4c3cc12] | 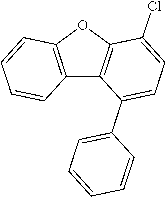 [SMILES: Clc1ccc(-c2ccccc2)c2c1oc1ccccc12] | 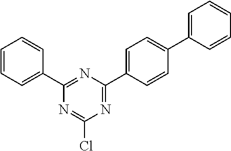 [SMILES: Clc1nc(-c2ccccc2)nc(-c2ccc(-c3ccccc3)cc2)n1] | 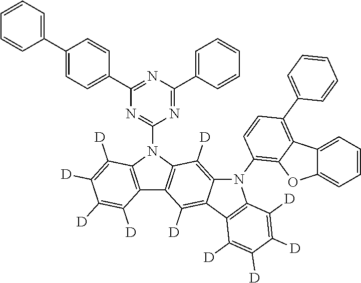 [SMILES: [2H]c1c([2H])c([2H])c2c(c1[2H])c1c([2H])c3c4c([2H])c([2H])c([2H])c([2H])c4n(-c4ccc(-c5ccccc5)c5c4oc4ccccc45)c3c([2H])c1n2-c1nc(-c2ccccc2)nc(-c2ccc(-c3ccccc3)cc2)n1] |
| | | | | 51% |
| 1-321 | 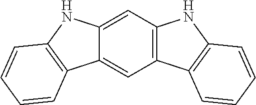 [SMILES: c1ccc2c(c1)[nH]c1cc3[nH]c4ccccc4c3cc12] | 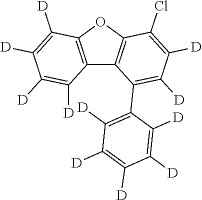 [SMILES: [2H]c1c([2H])c([2H])c(-c2c([2H])c([2H])c(Cl)c3oc4c([2H])c([2H])c([2H])c([2H])c4c23)c([2H])c1[2H]] | 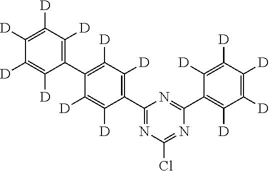 [SMILES: [2H]c1c([2H])c([2H])c(-c2nc(Cl)nc(-c3c([2H])c([2H])c(-c4c([2H])c([2H])c([2H])c([2H])c4[2H])c([2H])c3[2H])n2)c([2H])c1[2H]] | 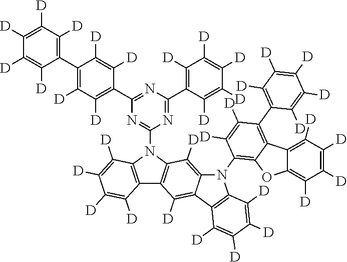 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-n3c4c([2H])c([2H])c([2H])c([2H])c4c4c([2H])c5c6c([2H])c([2H])c([2H])c([2H])c6n(-c6c([2H])c([2H])c(-c7c([2H])c([2H])c([2H])c([2H])c7[2H])c7c6oc6c([2H])c([2H])c([2H])c([2H])c67)c5c([2H])c43)n2)c([2H])c1[2H]] |
| | | | | 52% |
| 1-323 | 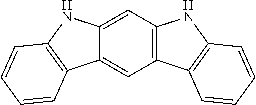 [SMILES: c1ccc2c(c1)[nH]c1cc3[nH]c4ccccc4c3cc12] | 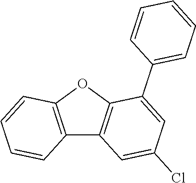 [SMILES: Clc1cc(-c2ccccc2)c2oc3ccccc3c2c1] | 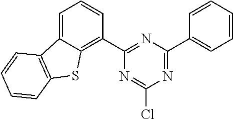 [SMILES: Clc1nc(-c2ccccc2)nc(-c2cccc3c2sc2ccccc23)n1] | 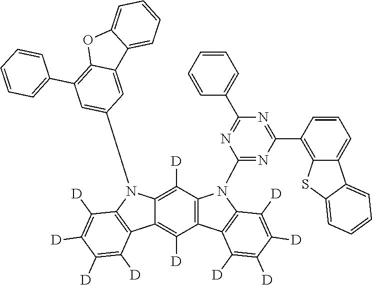 [SMILES: [2H]c1c([2H])c([2H])c2c(c1[2H])c1c([2H])c3c4c([2H])c([2H])c([2H])c([2H])c4n(-c4nc(-c5ccccc5)nc(-c5cccc6c5sc5ccccc56)n4)c3c([2H])c1n2-c1cc(-c2ccccc2)c2oc3ccccc3c2c1] |
| | | | | 48% |
| 1-324 | 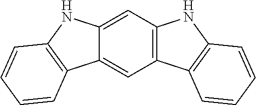 [SMILES: c1ccc2c(c1)[nH]c1cc3[nH]c4ccccc4c3cc12] | 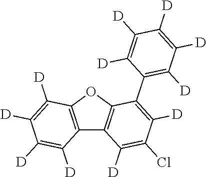 [SMILES: [2H]c1c([2H])c([2H])c(-c2c([2H])c(Cl)c([2H])c3c2oc2c([2H])c([2H])c([2H])c([2H])c23)c([2H])c1[2H]] | 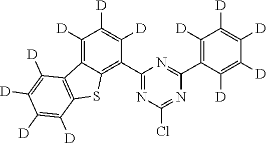 [SMILES: [2H]c1c([2H])c([2H])c(-c2nc(Cl)nc(-c3c([2H])c([2H])c([2H])c4c3sc3c([2H])c([2H])c([2H])c([2H])c34)n2)c([2H])c1[2H]] | 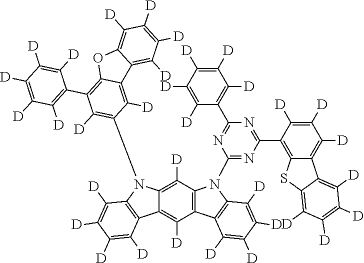 [SMILES: [2H]c1c([2H])c([2H])c(-c2nc(-c3c([2H])c([2H])c([2H])c4c3sc3c([2H])c([2H])c([2H])c([2H])c34)nc(-n3c4c([2H])c([2H])c([2H])c([2H])c4c4c([2H])c5c6c([2H])c([2H])c([2H])c([2H])c6n(-c6c([2H])c(-c7c([2H])c([2H])c([2H])c([2H])c7[2H])c7oc8c([2H])c([2H])c([2H])c([2H])c8c7c6[2H])c5c([2H])c43)n2)c([2H])c1[2H]] |
| | | | | 51% |
| 1-326 | 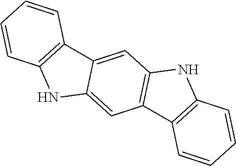 [SMILES: c1ccc2c(c1)[nH]c1cc3c(cc12)[nH]c1ccccc13] | 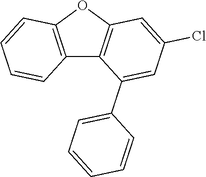 [SMILES: Clc1cc(-c2ccccc2)c2c(c1)oc1ccccc12] | 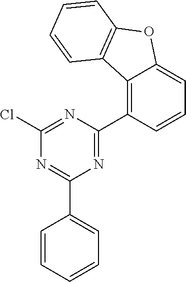 [SMILES: Clc1nc(-c2ccccc2)nc(-c2cccc3oc4ccccc4c23)n1] | 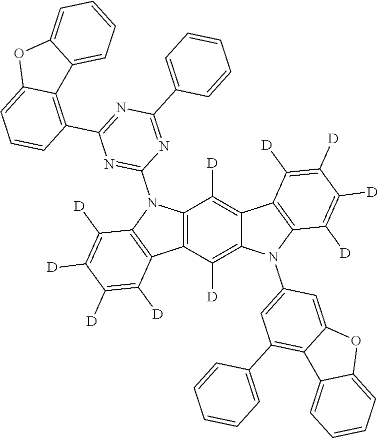 [SMILES: [2H]c1c([2H])c([2H])c2c(c1[2H])c1c([2H])c3c(c([2H])c1n2-c1cc(-c2ccccc2)c2c(c1)oc1ccccc12)c1c([2H])c([2H])c([2H])c([2H])c1n3-c1nc(-c2ccccc2)nc(-c2cccc3oc4ccccc4c23)n1] |
| | | | | 52% |
| 1-327 | 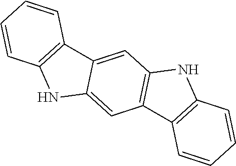 [SMILES: c1ccc2c(c1)[nH]c1cc3c(cc12)[nH]c1ccccc13] | 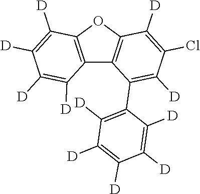 [SMILES: [2H]c1c([2H])c([2H])c(-c2c([2H])c(Cl)c([2H])c3oc4c([2H])c([2H])c([2H])c([2H])c4c23)c([2H])c1[2H]] | 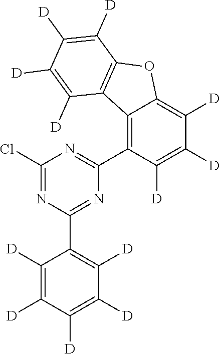 [SMILES: [2H]c1c([2H])c([2H])c(-c2nc(Cl)nc(-c3c([2H])c([2H])c([2H])c4oc5c([2H])c([2H])c([2H])c([2H])c5c34)n2)c([2H])c1[2H]] | 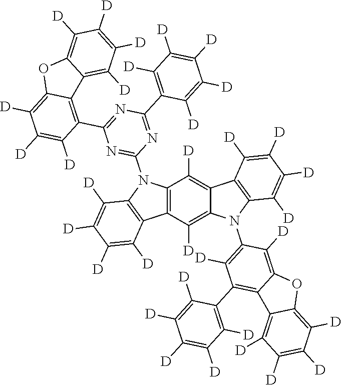 [SMILES: [2H]c1c([2H])c([2H])c(-c2nc(-c3c([2H])c([2H])c([2H])c4oc5c([2H])c([2H])c([2H])c([2H])c5c34)nc(-n3c4c([2H])c([2H])c([2H])c([2H])c4c4c([2H])c5c(c([2H])c43)c3c([2H])c([2H])c([2H])c([2H])c3n5-c3c([2H])c(-c4c([2H])c([2H])c([2H])c([2H])c4[2H])c4c(oc5c([2H])c([2H])c([2H])c([2H])c54)c3[2H])n2)c([2H])c1[2H]] |
| | | | | 52% |

Synthesis results for the compounds described in Preparation Example 1 to Preparation Example 5, and Table 1 to Table 5 are shown in the following Table 6 and Table 7. The following Table 6 shows measurement values of 1H NMR (CDCl₃, 300 MHz), and the following Table 7 shows measurement values of FD-mass spectrometry (FD-MS: field desorption mass spectrometry).

TABLE 6

| Compound No. | ¹H NMR (CDCl₃, 300 MHz) |
|---|---|
| 1-1 | δ = 8.55 (d, 2H), 8.36 (d, 4H), 8.12 (d, 1H), 7.98-7.94 (m, 3H), 7.79- |
| | 7.75 (m, 3H), 7.65 (s, 1H), 7.54-7.31 (m, 15H), 7.16 (t, 2H) |
| 1-2 | δ = 8.55 (d, 2H), 8.36 (d, 4H), 8.12 (d, 1H), 7.98-7.94 (m, 4H), 7.79 (d, |
| | 2H), 7.57-7.31 (m, 16H), 7.16 (t, 2H) |
| 1-3 | δ = 8.55 (d, 2H), 8.36 (d, 4H), 8.12 (d, 1H), 7.98-7.94 (m, 3H), 7.79 (d, |
| | 2H), 7.64-7.39 (m, 17H), 7.16 (t, 2H) |
| 1-4 | δ = 8.55 (d, 2H), 8.36 (d, 4H), 8.12 (d, 1H), 7.98-7.94 (m, 3H), 7.71 (s, |
| | 1H), 7.65 (s, 1H), 7.57-7.31 (m, 17H), 7.16 (t, 2H) |
| 1-5 | δ = 8.55 (d, 2H), 8.36 (d, 4H), 8.12 (d, 1H), 8.02 (d, 1H), 7.98-7.94 (m, |
| | 3H), 7.57-7.31 (m, 18H), 7.16 (t, 2H), |
| 1-6 | δ = 8.55 (d, 2H), 8.36 (d, 4H), 8.12 (d, 1H), 7.98-7.94 (m, 3H), 7.75- |
| | 7.72 (m, 3H), 7.57-7.35 (m, 16H), 7.16 (t, 2H) |
| 1-11 | δ = 8.55 (d, 2H), 8.36 (d, 4H), 8.12 (d, 1H), 7.98-7.94 (m, 3H), 7.82- |
| | 7.79 (m, 3H), 7.69 (d, 1H), 7.57-7.35 (m, 14H), 7.25 (d, 1H), 7.16 (t, |
| | 2H) |
| 1-12 | δ = 8.55 (d, 2H), 8.36 (d, 4H), 8.12 (d, 1H), 7.98-7.75 (m, 8H), 7.57- |
| | 7.35 (13H), 7.25 (d, 1H), 7.16 (t, 2H) |
| 1-13 | δ = 8.55 (d, 2H), 8.36 (d, 4H), 8.12 (d, 1H), 8.03-7.94 (m, 4H), 7.82- |
| | 7.75 (m, 4H), 7.57-7.35 (m, 13H), 7.25 (d, 1H), 7.16 (t, 2H) |
| 1-14 | δ = 8.55 (d, 2H), 8.36 (d, 4H), 8.12-7.94 (m, 6H), 7.57-7.35 (m, 16H), |
| | 7.25 (d, 1H), 7.16 (t, 2H) |
| 1-15 | δ = 8.55 (d, 2H), 8.36 (d, 4H), 8.12-8.02 (m, 3H), 7.94 (d, 2H), 7.74 (d, |
| | 1H), 7.57-7.31 (m, 17H), 7.16 (t, 2H) |
| 1-17 | δ = 8.55 (d, 2H), 8.36 (d, 4H), 8.12 (d, 1H), 7.94-7.74 (m, 8H), 7.50- |
| | 7.35 (m, 14H), 7.16 (t, 2H) |
| 1-21 | δ = 8.55 (d, 2H), 8.36 (d, 4H), 8.12 (d, 1H), 7.94-7.75 (m, 7H), 7.57- |
| | 7.35 (m, 14H), 7.25 (d, 1H) 7.16 (t, 2H), |
| 1-22 | δ = 8.55 (d, 2H), 8.36 (d, 4H), 8.12 (d, 1H), 7.94 (d, 2H), 7.82-7.79 (m, |
| | 3H), 7.69 (d, 1H), 7.57-7.35 (m, 15H), 7.25 (d, 1H), 7.16 (t, 2H) |
| 1-23 | δ = 8.55 (d, 2H), 8.36 (d, 4H), 8.12 (d, 1H), 7.98-7.94 (m, 3H), 7.79- |
| | 7.75 (m, 3H), 7.65 (s, 1H), 7.57-7.25 (m, 19H), 7.16 (t, 2H) |
| 1-24 | δ = 8.55 (d, 2H), 8.36 (d, 4H), 8.12 (d, 1H), 7.98-7.94 (m, 4H), 7.79- |
| | 7.73 (m, 4H), 7.57-7.35 (m, 18H), 7.16 (t, 2H) |
| 1-27 | δ = 8.55 (d, 2H), 8.36 (m, 2H), 8.12 (d, 1H), 7.98-7.94 (m, 5H), 7.79- |
| | 7.75 (m, 5H), 7.65 (s, 1H), 7.57-7.35 (m, 17H), 7.16 (t, 2H) |
| 1-33 | δ = 8.55 (d, 2H), 8.36 (m, 2H), 8.12 (d, 1H), 7.98-7.94 (m, 4H), 7.82- |
| | 7.65 (m, 6H), 7.57-7.31 (m, 16H), 7.16 (t, 2H) |
| 1-34 | δ = 8.55 (d, 2H), 8.36 (m, 2H), 8.12 (d, 1H), 7.98-7.94 (m, 5H), 7.82- |
| | 7.79 (m, 3H), 7.69 (d, 1H), 7.57-7.31 (m, 17H), 7.16 (t, 2H) |
| 1-35 | δ = 8.55 (d, 2H), 8.36 (m, 2H), 8.12 (d, 1H), 7.98-7.94 (m, 4H), 7.82- |
| | 7.79 (m, 3H), 7.57-7.31 (m, 19H), 7.16 (t, 2H) |
| 1-36 | δ = 8.55 (d, 2H), 8.36 (m, 2H), 8.12 (d, 1H), 7.98-7.94 (m, 4H), 7.82 (d, |
| | 1H), 7.71-7.65 (m, 3H), 7.57-7.31 (m, 18H), 7.16 (t, 2H) |
| 1-37 | δ = 8.55 (d, 2H), 8.36 (m, 2H), 8.12 (d, 1H), 8.02-7.94 (m, 5H), 7.82 (d, |
| | 1H), 7.69 (d, 1H), 7.57-7.31 (m, 19H), 7.16 (t, 2H) |
| 1-38 | δ = 8.55 (d, 2H), 8.36 (m, 2H), 8.12 (d, 1H), 7.98-7.94 (m, 4H), 7.82- |
| | 7.72 (m, 5H), 7.59-7.31 (m, 17H), 7.16 (t, 2H) |
| 1-43 | δ = 8.55 (d, 2H), 8.36 (m, 2H), 8.12 (d, 1H), 7.98-7.94 (m, 4H), 7.82- |
| | 7.79 (m, 4H), 7.69 (d, 2H), 7.57-7.31 (m, 16H), 7.16 (t, 2H) |
| 1-44 | δ = 8.55 (d, 2H), 8.36 (m, 2H), 8.12 (d, 1H), 7.98-7.75 (m, 11H), 7.57- |
| | 7.31 (m, 15H), 7.16 (t, 2H) |
| 1-47 | δ = 8.55 (d, 2H), 8.36 (m, 2H), 8.12-7.94 (m, 6H), 7.82 (d, 1H), 7.74- |
| | 7.69 (m, 2H), 7.57-7.31 (m, 18H), 7.16 (t, 2H) |
| 1-48 | δ = 8.55 (d, 2H), 8.36 (m, 2H), 8.12 (d, 1H), 8.03-7.94 (m, 4H), 7.82- |
| | 7.69 (m, 7H), 7.61-7.31 (m, 15H), 7.16 (t, 2H) |
| 1-51 | δ = 8.55 (d, 2H), 8.36 (m, 2H), 8.12-7.94 (m, 6H), 7.82 (d, 1H), 7.69 (d, |
| | 1H), 7.57-7.31 (m, 19H), 7.16 (t, 2H) |
| 1-52 | δ = 8.55 (d, 2H), 8.36 (m, 2H), 8.12 (d, 1H), 8.03-7.94 (m, 4H), 7.82- |
| | 7.69 (m, 6H), 7.57-7.25 (m, 16H), 7.16 (t, 2H) |
| 1-55 | δ = 8.55 (d, 2H), 8.36 (m, 2H), 8.12 (d, 1H), 7.98-7.94 (m, 4H), 7.82- |
| | 7.65 (m, 6H), 7.57-7.25 (m, 20H), 7.16 (t, 2H) |
| 1-56 | δ = 8.55 (d, 2H), 8.36 (m, 2H), 8.12 (d, 1H), 7.98-7.94 (m, 5H), 7.82- |
| | 7.31 (m, 25H), 7.16 (t, 2H) |
| 1-61 | δ = 8.55 (d, 2H), 8.36 (m, 2H), 8.12 (d, 1H), 7.98-7.75 (m, 10H), 7.65 (s, |
| | 1H), 7.54-7.25 (m, 19H), 7.16 (t, 2H) |
| 1-62 | δ = 8.55 (d, 2H), 8.36 (m, 2H), 8.12 (d, 1H), 7.98-7.73 (m, 12H), 7.65- |
| | 7.31 (m, 18H), 7.16 (t, 2H) |
| 1-65 | δ = 8.55 (d, 2H), 8.36 (m, 2H), 8.12 (d, 1H), 8.03-7.94 (m, 5H), 7.82- |
| | 7.75 (m, 5H), 7.65 (s, 1H), 7.57-7.31 (m, 15H), 7.16 (t, 2H) |
| 1-66 | δ = 8.55 (d, 2H), 8.36 (m, 2H), 8.12 (d, 1H), 8.03-7.94 (m, 5H), 7.82- |
| | 7.69 (m, 6H), 7.57-7.31 (m, 15H), 7.16 (t, 2H) |
| 1-67 | δ = 8.55 (d, 2H), 8.36 (m, 2H), 8.12 (d, 1H), 8.03-7.94 (m, 5H), 7.82- |
| | 7.75 (m, 5H). 7.65 (s, 1H), 7.57-7.25 (m, 19H), 7.16 (t, 2H) |
| 1-68 | δ = 8.55 (d, 2H), 8.36 (m, 2H), 8.12 (d, 1H), 8.03-7.94 (m, 6H), 7.82- |
| | 7.73 (m, 6H), 7.57-7.31 (m, 18H), 7.16 (t, 2H) |
| 1-77 | δ = 8.55 (d, 2H), 8.45 (d, 1H), 8.36 (m, 2H), 8.12 (d, 1H), 8.03-7.93 (m, |
| | 6H), 7.79-7.65 (m, 5H), 7.57-7.31 (m, 14H), 7.16 (t, 2H) |
| 1-78 | δ = 8.55 (d, 2H), 8.45 (d, 1H), 8.36 (m, 2H), 8.12 (d, 1H), 8.03-7.93 (m, |
| | 7H), 7.79 (d, 2H), 7.68 (t, 1H), 7.57-7.31 (m, 15H), 7.16 (t, 2H) |
| 1-79 | δ = 8.55 (d, 2H), 8.45 (d, 1H), 8.36 (m, 2H), 8.12 (d, 1H), 8.03-7.93 (m, |
| | 6H), 7.79 (d, 2H), 7.68-7.31 (m, 17H), 7.16 (t, 2H) |
| 1-80 | δ = 8.55 (d, 2H), 8.45 (d, 1H), 8.36 (m, 2H), 8.12 (d, 1H), 8.03-7.93 (m, |
| | 6H), 7.71-7.65 (m, 3H), 7.57-7.31 (m, 16H), 7.16 (t, 2H) |
| 1-81 | δ = 8.55 (d, 2H), 8.45 (d, 1H), 8.36 (m, 2H), 8.12 (d, 1H), 8.03-7.93 (m, |
| | 7H), 7.68 (t, 1H), 7.57-7.31 (m, 17H), 7.16 (t, 2H) |
| 1-82 | δ = 8.55 (d, 2H), 8.45 (d, 1H), 8.36 (m, 2H), 8.12 (d, 1H), 8.03-7.93 (m, |
| | 6H), 7.75-7.68 (m, 4H), 7.59-7.31 (m, 15H), 7.16 (t, 2H) |
| 1-83 | δ = 8.55 (d, 2H), 8.45 (d, 1H), 8.36 (m, 2H), 8.12 (d, 1H), 8.03 (d, 1H), |
| | 7.94-7.93 (m, 5H), 7.82-7.79 (m, 3H), 7.69-7.68 (t, 2H), 7.57-7.31 (m, |
| | 14H), 7.16 (t, 2H) |
| 1-84 | δ = 8.55 (d, 2H), 8.45 (d, 1H), 8.36 (m, 2H), 8.12 (d, 1H), 8.03 (d, 1H), |
| | 7.94-7.68 (m, 11H), 7.57-7.31 (m, 13H), 7.16 (t, 2H) |
| 1-87 | δ = 8.55 (d, 2H), 8.45 (d, 1H), 8.36 (m, 2H), 8.12 (d, 1H), 8.03-7.93 (m, |
| | 6H), 7.82-7.79 (m, 3H), 7.69-7.68 (t, 2H), 7.57-7.35 (m, 13H), 7.25 (d, |
| | 1H), 7.16 (t, 2H) |
| 1-88 | δ = 8.55 (d, 2H), 8.45 (d, 1H), 8.36 (m, 2H), 8.12 (d, 1H), 8.03-7.68 (m, |
| | 12H), 7.57-7.35 (m, 12H), 7.25 (d, 1H), 7.16 (t, 2H) |
| 1-91 | δ = 8.55 (d, 2H), 8.45 (d, 1H), 8.36 (m, 2H), 8.12-8.02 (m, 4H), 7.94- |
| | 7.93 (m, 4H), 7.68-7.31 (m, 18H), 7.16 (t, 2H), |
| 1-92 | δ = 8.55 (d, 2H), 8.45 (d, 1H), 8.36 (m, 2H), 8.12 (d, 1H), 8.03 (d, 2H), |
| | 7.94-7.93 (m, 4H), 7.82-7.31 (m, 19H), 7.16 (t, 2H) |
| 1-95 | δ = 8.55 (d, 2H), 8.45 (d, 1H), 8.36 (m, 2H), 8.12-8.02 (m, 4H), 7.94- |
| | 7.93 (m, 4H), 7.68 (t, 1H), 7.57-7.35 (m, 16H), 7.25 (d, 1H), 7.16 (t, |
| | 2H) |
| 1-96 | δ = 8.55 (d, 2H), 8.45 (d, 1H), 8.36 (m, 2H), 8.12 (d, 1H), 8.03 (d, 2H), |
| | 7.94-7.93 (m, 4H), 7.82-7.68 (m, 5H), 7.57-7.35 (m, 13H), 7.25 (d, 1H), |
| | 7.16 (t, 2H) |
| 1-99 | δ = 8.55 (d, 2H), 8.45 (d, 1H), 8.36 (m, 2H), 8.12 (d, 1H), 8.03-7.93 (m, |
| | 6H), 7.79-7.65 (m, 5H), 7.57-7.16 (m, 20H). |
| 1-100 | δ = 8.55 (d, 2H), 8.45 (d, 1H), 8.36 (m, 2H), 8.12 (d, 1H), 8.03-7.93 (m, |
| | 7H), 7.79-7.35 (m, 22H), 7.16 (t, 2H) |
| 1-105 | δ = 8.55 (d, 2H), 8.45 (d, 1H), 8.36 (m, 2H), 8.12 (m, 2H), 7.99-7.92 (m, |
| | 6H), 7.79-7.75 (m, 3H), 7.65 (s, 1H), 7.57-7.16 (m, 20H) |
| 1-106 | δ = 8.55 (d, 2H), 8.45 (d, 1H), 8.36 (m, 2H), 8.12 (m, 2H), 7.99-7.92 (m, |
| | 7H), 7.79-7.73 (m, 4H), 7.61-7.31 (m, 17H), 7.16 (t, 2H) |
| 1-109 | δ = 8.55 (d, 2H), 8.45 (d, 1H), 8.36 (m, 2H), 8.24-8.20 (m, 2H), 8.12 (d, |
| | 1H), 7.98-7.93 (m, 5H), 7.79-7.75 (m, 3H), 7.65 (s, 1H), 7.57-7.31 (m, |
| | 14H), 7.16 (t, 2H) |
| 1-121 | δ = 8.55 (d, 2H), 8.36 (m, 2H), 8.12 (m, 1H), 7.98-7.90 (m, 4H), 7.79- |
| | 7.75 (m, 4H), 7.65 (m, 2H), 7.57-7.28 (m, 16H), 7.16 (t, 2H), 1.69 (s, |
| | 6H) |
| 1-122 | δ = 8.55 (d, 2H), 8.36 (m, 2H), 8.12 (d, 1H), 7.98-7.90 (m, 5H), 7.79- |
| | 7.78 (m, 3H), 7.65 (d, 1H), 7.57-7.28 (m, 17H), 7.16 (t, 2H), 1.69 (s, |
| | 6H) |
| 1-123 | δ = 8.55 (d, 2H), 8.36 (m, 2H), 8.12 (d, 1H), 7.98-7.90 (m, 4H), 7.79- |
| | 7.78 (m, 3H), 7.57-7.28 (m, 19H), 7.16 (t, 2H), 1.69 (s, 6H) |
| 1-124 | δ = 8.55 (d, 2H), 8.36 (m, 2H), 8.12 (d, 1H), 7.98-7.90 (m, 4H), 7.78 (d, |
| | 1H), 7.71-7.65 (m, 3H), 7.57-7.28 (m, 18H), 7.16 (t, 2H), 1.69 (s, 6H) |
| 1-131 | δ = 8.55 (d, 2H), 8.36 (m, 2H), 8.12 (d, 1H), 7.98-7.90 (m, 4H), 7.82- |
| | 7.78 (m, 4H), 7.57-7.25 (m, 18H), 7.16 (t, 2H), 1.69 (s, 6H) |
| 1-132 | δ = 8.55 (d, 2H), 8.36 (m, 2H), 8.12 (d, 1H), 7.98-7.75 (m, 10H), 7.65 (d, |
| | 1H), 7.57-7.25 (m, 15H), 7.16 (t, 2H), 1.69 (s, 6H) |
| 1-135 | δ = 8.55 (d, 2H), 8.36 (m, 2H), 8.12-8.02 (m, 3H), 7.94-7.90 (m, 3H), |
| | 7.78-7.74 (m, 2H), 7.65-7.28 (m, 19H), 7.16 (t, 2H), 1.69 (s, 6H) |
| 1-136 | δ = 8.55 (d, 2H), 8.36 (m, 2H), 8.12 (d, 1H), 8.03 (d, 1H), 7.94-7.90 (m, |
| | 3H), 7.82-7.74 (m, 6H), 7.65-7.31 (m, 16H), 7.16 (t, 2H). 1.69 (s, 6H) |
| 1-141 | δ = 8.55 (d, 2H), 8.36 (m, 2H), 8.12 (d, 1H), 7.94-7.75 (m, 9H), 7.65 (d, |
| | 1H), 7.57-7.25 (m, 16H), 7.16 (t, 2H), 1.69 (s, 6H) |
| 1-142 | δ = 8.55 (d, 2H), 8.36 (m, 2H), 8.12 (d, 1H), 7.94-7.90 (m, 3H), 7.82- |
| | 7.78 (m, 4H), 7.57-7.25 (m, 19H), 7.16 (t, 2H), 1.69 (s, 6H) |
| 1-143 | δ = 8.55 (d, 2H), 8.36 (m, 2H), 8.12 (d, 1H), 7.98-7.90 (m, 4H), 7.79- |
| | 7.75 (m, 4H), 7.65 (m, 2H), 7.57-7.25 (m, 20H), 7.16 (t, 2H), 1.69 (s, |
| | 6H) |
| 1-144 | δ = 8.55 (d, 2H), 8.36 (m, 2H), 8.12 (d, 1H), 7.98-7.90 (m, 5H), 7.78- |
| | 7.31 (m, 25H), 7.16 (t, 2H), 1.69 (s, 6H) |
| 1-153 | δ = 8.55 (d, 2H), 8.36 (d, 2H), 8.12-8.09 (m, 2H), 7.98-7.89 (m, 4H), |
| | 7.79-7.75 (m, 3H), 7.65 (s, 1H), 7.55-7.28 (m, 15H), 7.16 (t, 2H), |
| | 1.69 (s, 6H) |
| 1-193 | δ = 8.55 (d, 1H), 8.36 (m, 4H), 8.19 (d, 1H), 7.98-7.94 (m, 4H), 7.79- |
| | 7.75 (m, 3H), 7.65 (s, 1H) 7.58-7.31 (m, 15H), 7.20-7.16 (m, 2H) |
| 1-194 | δ = 8.55 (d, 1H), 8.36 (m, 4H), 8.19 (d, 1H), 7.96-7.94 (m, 4H), 7.82- |
| | 7.79 (m, 3H), 7.69 (d, 1H), 7.58-7.31 (m, 15H), 7.20-7.16 (m, 2H) |
| 1-195 | δ = 8.55 (d, 1H), 8.36 (m, 4H), 8.19 (d, 1H), 8.03-7.94 (m, 5H), 7.82- |
| | 7.75 (m, 4H), 7.58-7.35 (m, 13H), 7.25-7.16 (m, 3H) |
| 1-196 | δ = 8.55 (d, 1H), 8.36 (m, 4H), 8.19 (d, 1H), 8.08-7.94 (m, 5H), 7.58- |
| | 7.41 (m, 17H), 7.25-7.16 (m, 3H), |
| 1-205 | δ = 8.55 (d, 1H), 8.36 (m, 2H), 8.19 (d, 1H), 7.98-7.94 (m, 5H), 7.79- |
| | 7.75 (m, 4H), 7.58-7.16 (m, 22H) |
| 1-206 | δ = 8.55 (d, 1H), 8.36 (m, 2H), 8.19 (d, 1H), 7.96-7.75 (m, 11H), 7.55- |
| | 7.16 (m, 20H) |
| 1-207 | δ = 8.55 (d, 1H), 8.36 (m, 2H), 8.19 (d, 1H), 8.08-7.94 (m, 6H), 7.75 (d, |
| | 2H), 7.58-7.35 (m, 18H), 7.25-7.16 (m, 5H) |
| 1-208 | δ = 8.55 (d, 1H), 8.36 (m, 2H), 8.19 (d, 1H), 8.03-7.94 (m, 4H), 7.82- |
| | 7.75 (m, 6H), 7.58-7.35 (m, 16H), 7.25-7.16 (m, 5H) |
| 1-221 | δ = 8.55 (d, 1H), 8.45 (d, 1H), 8.36 (m, 2H), 8.19 (d, 1H), 8.03-7.93 (m, |
| | 5H), 7.79-7.65 (m, 5H), 7.58-7.31 (m, 16H), 7.20-7.16 (m, 2H) |
| 1-222 | δ = 8.55 (d, 1H), 8.45 (d, 1H), 8.36 (m, 2H), 8.19 (d, 1H), 8.03 (d, 1H), |
| | 7.94-7.93 (m, 4H), 7.82-7.79 (m, 3H) 7.69-7.68 (m, 2H), 7.58-7.35 (m, |
| | 16H), 7.20-7.16 (m, 2h) |
| 1-223 | δ = 8.55 (d, 1H), 8.45 (d, 1H), 8.36 (m, 2H), 8.19 (d, 1H), 8.03-7.93 (m, |
| | 6H), 7.82-7.68 (m, 5H), 7.58-7.35 (m, 14H), 7.25-7.16 (m, 3H) |
| 1-224 | δ = 8.55 (d, 1H), 8.45 (d, 1H), 8.36 (m, 2H), 8.19 (d, 1H), 8.08-8.02 (m, |
| | 3H), 7.94-7.93 (m, 3H), 7.68 (t, 1H), 7.58-7.35 (m, 18H), 7.25-7.16 (m, |
| | 3H) |
| 1-229 | δ = 8.36 (m, 4H), 7.98 (d, 1H), 7.79-7.75 (m, 3H), 7.65 (s, 1H), 7.54- |
| | 7.31 (m, 12H) |
| 1-230 | δ = 8.55 (m, 2H), 8.12 (d, 1H), 7.94 (d, 2H), 7.57 (d, 1H), 7.35 (t, 2H), |
| | 7.16 (t, 2H) |
| 1-241 | δ = 8.36 (m, 4H), 8.02-7.98 (m, 2H), 7.54-7.31 (m, 15H) |
| 1-242 | δ = 8.55 (m, 2H), 8.12 (d, 1H), 7.94 (d, 2H), 7.57 (d, 1H), 7.35 (t, 2H), |
| | 7.16 (t, 2H) |
| 1-244 | δ = 8.36 (m, 4H), 7.98 (d, 1H), 7.75-7.72 (m, 3H), 7.59-7.31 (m, 13H) |
| 1-245 | δ = 8.55 (m, 2H), 8.12 (d, 1H), 7.94 (d, 2H), 7.57 (d, 1H), 7.35 (t, 2H), |
| | 7.16 (t, 2H) |
| 1-247 | δ = 8.36 (m, 4H), 7.94 (d, 1H), 7.82-7.79 (m, 3H), 7.69 (d, 1H), 7.57- |
| | 7.41 (m, 11H), 7.31 (d, 1H) |
| 1-248 | δ = 8.55 (m, 2H), 8.12 (d, 1H), 7.94 (d, 2H), 7.57 (d, 1H), 7.35 (t, 2H), |
| | 7.16 (t, 2H) |
| 1-265 | δ = 8.36 (m, 4H), 8.03-7.98 (m, 2H), 7.82-7.75 (m, 4H), 7.54-7.41 (m, |
| | 10H), 7.25 (d, 1H) |
| 1-266 | δ = 8.55 (m, 2H), 8.12 (d, 1H), 7.94 (d, 2H), 7.57 (d, 1H), 7.35 (t, 2H), |
| | 7.16 (t, 2H) |
| 1-268 | δ = 8.36 (m, 4H), 8.08-7.98 (m, 3H), 7.54-7.41 (m, 13H), 7.25 (d, 1H) |
| 1-269 | δ = 8.55 (m, 2H), 8.12 (d, 1H), 7.94 (d, 2H), 7.57 (d, 1H), 7.35 (t, 2H), |
| | 7.16 (t, 2H) |
| 1-316 | δ = 8.55 (m, 2H), 8.12 (d, 1H), 7.94 (d, 2H), 7.57 (d, 1H), 7.35 (t, 2H), |
| | 7.16 (t, 2H) |
| 1-317 | δ = 8.36 (m, 2H), 8.03-7.98 (m, 3H), 7.78-7.69 (m, 6H), 7.57-7.25 (m, |
| | 12H) |
| 1-319 | δ = 8.55 (m, 2H), 8.12 (d, 1H), 7.94 (d, 2H), 7.57 (d, 1H), 7.35 (t, 2H), |
| | 7.16 (t, 2H) |
| 1-320 | δ = 8.36 (m, 2H), 7.98-7.96 (m, 4H), 7.79-7.75 (m, 4H), 7.54-7.25 (m, |
| | 15H) |
| 1-322 | δ = 8.55 (d, 1H), 8.19 (d, 1H), 7.94 (d, 1H), 7.58-7.50 (m, 3H), 7.40- |
| | 7.35 (m, 2H), 7.20-7.16 (m, 2H) |
| 1-323 | δ = 8.55 (d, 1H), 8.45 (d, 1H), 8.36 (m, 2H), 7.98-7.92 (m, 3H), 7.71- |
| | 7.65 (m, 3H), 7.56-7.31 (m, 13H) |
| 1-325 | δ = 8.55 (d, 1H), 8.19 (d, 1H), 7.94 (d, 1H), 7.58-7.50 (m, 3H), 7.40- |
| | 7.35 (m, 2H), 7.20-7.16 (m, 2H). |
| 1-326 | 8.36 (m, 2H), 7.98 (d, 2H), 7.82-7.79 (m, 3H), 7.59-7.31 (m, 16H) |
| 1-331 | 9.60 (m 1H), 9.27(s, 1H), 8.55 (d, 2H), 8.36-8.33 (m, 3H), 8.15- |
| | 8.12 (m, 2H), 7.98-7.94 (m, 3H), 7.70-7.31 (m, 21H), 7.16 (t, 2H) |
| 1-332 | 9.60 (m 1H), 9.27 (s, 1H), 8.55 (d, 2H), 8.36-8.30 (m, 4H), 8.15- |
| | 8.12 (m, 2H), 7.98-7.94 (m, 4H), 7.70-7.31 (m, 21H), 7.16 (t, 2H) |
| 1-335 | 9.27 (s, 1H), 8.79 (d, 1H), 8.55 (d, 2H), 8.45 (d, 1H), 8.36-8.30 (m, |
| | 5H), 8.15-8.06 (m, 3H), 7.94-7.93 (m, 3H), 7.70-7.35 (m, 19H), 7.16 (t, |
| | 2H) |

TABLE 7

| Compound No. | FD-MS | Compound No. | FD-MS |
|---|---|---|---|
| 1-1 | m/z: 729.25 (C₅₁H₃₁N₅O = 729.84) | 1-2 | m/z: 729.25 (C₅₁H₃₁N₅O = 729.84) |
| 1-3 | m/z: 729.25 (C₅₁H₃₁N₅O = 729.84) | 1-4 | m/z: 729.25 (C₅₁H₃₁N₅O = 729.84) |
| 1-5 | m/z: 729.25 (C₅₁H₃₁N₅O = 729.84) | 1-6 | m/z: 729.25 (C₅₁H₃₁N₅O = 729.84) |
| 1-11 | m/z: 729.25 (C₅₁H₃₁N₅O = 729.84) | 1-12 | m/z: 729.25 (C₅₁H₃₁N₅O = 729.84) |
| 1-13 | m/z: 729.25 (C₅₁H₃₁N₅O = 729.84) | 1-14 | m/z: 729.25 (C₅₁H₃₁N₅O = 729.84) |
| 1-15 | m/z: 729.25 (C₅₁H₃₁N₅O = 729.84) | 1-17 | m/z: 729.25 (C₅₁H₃₁N₅O = 729.84) |
| 1-21 | m/z: 729.25 (C₅₁H₃₁N₅O = 729.84) | 1-22 | m/z: 729.25 (C₅₁H₃₁N₅O = 729.84) |
| 1-23 | m/z: 805.28 (C₅₇H₃₅N₅O = 805.94) | 1-24 | m/z: 805.28 (C₅₇H₃₅N₅O = 805.94) |
| 1-27 | m/z: 805.28 (C₅₇H₃₅N₅O = 805.94) | 1-33 | m/z: 819.26 (C₅₇H₃₃N₅O₂ = 819.92) |
| 1-34 | m/z: 819.26 (C₅₇H₃₃N₅O₂ = 819.92) | 1-35 | m/z: 819.26 (C₅₇H₃₃N₅O₂ = 819.92) |
| 1-36 | m/z: 819.26 (C₅₇H₃₃N₅O₂ = 819.92) | 1-37 | m/z: 819.26 (C₅₇H₃₃N₅O₂ = 819.92) |
| 1-38 | m/z: 819.26 (C₅₇H₃₃N₅O₂ = 819.92) | 1-43 | m/z: 819.26 (C₅₇H₃₃N₅O₂ = 819.92) |
| 1-44 | m/z: 819.26 (C₅₇H₃₃N₅O₂ = 819.92) | 1-47 | m/z: 819.26 (C₅₇H₃₃N₅O₂ = 819.92) |
| 1-48 | m/z: 819.26 (C₅₇H₃₃N₅O₂ = 819.92) | 1-51 | m/z: 819.26 (C₅₇H₃₃N₅O₂ = 819.92) |
| 1-52 | m/z: 819.26 (C₅₇H₃₃N₅O₂ = 819.92) | 1-55 | m/z: 895.29 (C₆₃H₃₇N₅O₂ = 896.02) |
| 1-56 | m/z: 895.29 (C₆₃H₃₇N₅O₂ = 896.02) | 1-61 | m/z: 895.29 (C₆₃H₃₇N₅O₂ = 896.02) |
| 1-62 | m/z: 895.29 (C₆₃H₃₇N₅O₂ = 896.02) | 1-65 | m/z: 819.26 (C₅₇H₃₃N₅O₂ = 819.92) |
| 1-66 | m/z: 819.26 (C₅₇H₃₃N₅O₂ = 819.92) | 1-67 | m/z: 895.29 (C₆₃H₃₇N₅O₂ = 896.02) |
| 1-68 | m/z: 895.29 (C₆₃H₃₇N₅O₂ = 896.02) | 1-77 | m/z: 835.24 (C₅₇H₃₃NOS = 835.98) |
| 1-78 | m/z: 835.24 (C₅₇H₃₃N₅OS = 835.98) | 1-79 | m/z: 835.24 (C₅₇H₃₃N₅OS = 835.98) |
| 1-80 | m/z: 835.24 (C₅₇H₃₃N₅OS = 835.98) | 1-81 | m/z: 835.24 (C₅₇H₃₃N₅OS = 835.98) |
| 1-82 | m/z: 835.24 (C₅₇H₃₃N₅OS = 835.98) | 1-83 | m/z: 835.24 (C₅₇H₃₃N₅OS = 835.98) |
| 1-84 | m/z: 835.24 (C₅₇H₃₃N₅OS = 835.98) | 1-87 | m/z: 835.24 (C₅₇H₃₃N₅OS = 835.98) |
| 1-88 | m/z: 835.24 (C₅₇H₃₃N₅OS = 835.98) | 1-91 | m/z: 835.24 (C₅₇H₃₃N₅OS = 835.98) |
| 1-92 | m/z: 835.24 (C₅₇H₃₃N₅OS = 835.98) | 1-95 | m/z: 835.24 (C₅₇H₃₃N₅OS = 835.98) |
| 1-96 | m/z: 835.24 (C₅₇H₃₃N₅OS = 835.98) | 1-99 | m/z: 911.27 (C₆₃H₃₇N₅OS = 912.08) |
| 1-100 | m/z: 911.27 (C₆₃H₃₇N₅OS = 912.08) | 1-105 | m/z: 911.27 (C₆₃H₃₇N₅OS = 912.08) |
| 1-106 | m/z: 911.27 (C₆₃H₃₇N₅OS = 912.08) | 1-109 | m/z: 835.24 (C₅₇H₃₃N₅OS = 835.24) |
| 1-121 | m/z: 845.32 (C₆₀H₃₉N₅O = 846.01) | 1-122 | m/z: 845.32 (C₆₀H₃₉N₅O = 846.01) |
| 1-123 | m/z: 845.32 (C₆₀H₃₉N₅O = 846.01) | 1-124 | m/z: 845.32 (C₆₀H₃₉N₅O = 846.01) |
| 1-131 | m/z: 845.32 (C₆₀H₃₉N₅O = 846.01) | 1-132 | m/z: 845.32 (C₆₀H₃₉N₅O = 846.01) |
| 1-135 | m/z: 845.32 (C₆₀H₃₉N₅O = 846.01) | 1-136 | m/z: 845.32 (C₆₀H₃₉N₅O = 846.01) |
| 1-141 | m/z: 845.32 (C₆₀H₃₉N₅O = 846.01) | 1-142 | m/z: 845.32 (C₆₀H₃₉N₅O = 846.01) |
| 1-143 | m/z: 921.35 (C₆₆H₄₃N₅O = 922.10) | 1-144 | m/z: 921.35 (C₆₆H₄₃N₅O = 922.10) |
| 1-153 | m/z: 845.32 (C₆₀H₃₉N₅O = 845.32) | 1-193 | m/z: 729.25 (C₅₁H₃₁N₅O = 729.84) |
| 1-194 | m/z: 729.25 (C₅₁H₃₁N₅O = 729.84) | 1-195 | m/z: 729.25 (C₅₁H₃₁N₅O = 729.84) |
| 1-196 | m/z: 729.25 (C₅₁H₃₁N₅O = 729.84) | 1-205 | m/z: 805.28 (C₅₇H₃₅N₅O = 805.94) |
| 1-206 | m/z: 805.28 (C₅₇H₃₅N₅O = 805.94) | 1-207 | m/z: 805.28 (C₅₇H₃₅N₅O = 805.94) |
| 1-208 | m/z: 805.28 (C₅₇H₃₅N₅O = 805.94) | 1-221 | m/z: 835.24 (C₅₇H₃₃NOS = 835.98) |
| 1-222 | m/z: 835.24 (C₅₇H₃₃NOS = 835.98) | 1-223 | m/z: 835.24 (C₅₇H₃₃NOS = 835.98) |
| 1-224 | m/z: 835.24 (C₅₇H₃₃N₅OS = 835.98) | 1-229 | m/z: 739.32 (C₅₁H₂₁D₁₀N₅O = 739.90) |
| 1-230 | m/z: 750.38 (C₅₁H₁₀D₂₁N₅O = 750.97) | 1-231 | m/z: 760.45 (C51D31N5O = 761.03) |
| 1-241 | m/z: 739.32 (C₅₁H₂₁D₁₀N₅O = 739.90) | 1-242 | m/z: 750.38 (C₅₁H₁₀D₂₁N₅O = 750.97) |
| 1-243 | m/z: 760.45 (C51D31N5O = 761.03) | 1-244 | m/z: 739.32 (C₅₁H₂₁D₁₀N₅O = 739.90) |
| 1-245 | m/z: 750.38 (C₅₁H₁₀D₂₁N₅O = 750.97) | 1-246 | m/z: 760.45 (C51D31N5O = 761.03) |
| 1-247 | m/z: 739.32 (C₅₁H₂₁D₁₀N₅O = 739.90) | 1-248 | m/z: 750.38 (C₅₁H₁₀D₂₁N₅O = 750.97) |
| 1-249 | m/z: 760.45 (C51D31N5O = 761.03) | 1-265 | m/z: 739.32 (C₅₁H₂₁D₁₀N₅O = 739.90) |
| 1-266 | m/z: 750.38 (C₅₁H₁₀D₂₁N₅O = 750.97) | 1-267 | m/z: 760.45 (C₅₁D₃₁N₅O = 761.03) |
| 1-268 | m/z: 739.32 (C₅₁H₂₁D₁₀N₅O = 739.90) | 1-269 | m/z: 750.38 (C₅₁H₁₀D₂₁N₅O = 750.97) |
| 1-270 | m/z: 760.45 (C₅₁D₃₁N₅O = 761.03) | 1-316 | m/z: 842.41 (C₅₇H₁₀D₂₃N₅O₂ = 843.06) |
| 1-317 | m/z: 829.33 (C₅₇H₂₃D₁₀N₅O₂ = 829.99) | 1-318 | m/z: 852.47 (C₅₇D₃₃N₅O₂ = 853.13) |
| 1-319 | m/z: 830.44 (C₅₇H₁₀D₂₅N₅O = 831.09) | 1-320 | m/z: 815.35 (C₅₇H₂₅D₁₀N₅O = 816.00) |
| 1-321 | m/z: 840.50 (C₅₇D₃₅N₅O = 841.15) | 1-322 | m/z: 858.38 (C₅₇H₁₀D₂₃N₅OS = 859.13) |
| 1-323 | m/z: 845.30 (C₅₇H₂₃D₁₀N₅OS = 846.05) | 1-324 | m/z: 868.45 (C₅₇D₃₃NOS = 869.19) |
| 1-325 | m/z: 842.41 (C₅₇H₁₀D₂₃N₅O₂ = 843.06) | 1-326 | m/z: 829.33 (C₅₇H₂₃D₁₀N₅O₂S = 829.99) |
| 1-327 | m/z: 852.47 (C₅₇D₃₃N₅O₂ = 853.13) | 1-331 | m/z: 879.30 (C₆₃H₃₇N₅O = 880.02) |
| 1-332 | m/z: 879.30 (C₆₃H₃₇N₅O = 880.02) | 1-335 | m/z: 895.28 (C₆₃H₃₇N₅S = 896.08) |

### Preparation Example 6. Preparation of Compound 2-1

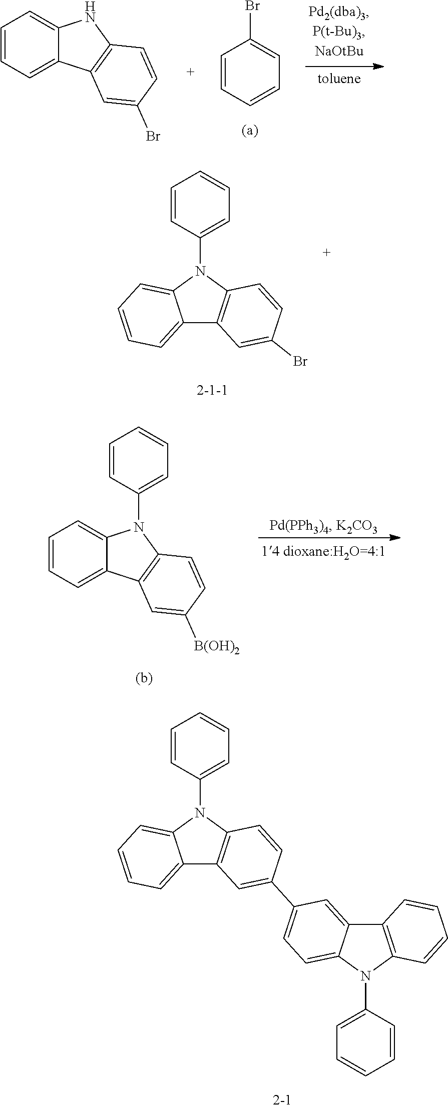

### Preparation Example 6-1. Preparation of Compound 2-1-1

In a reaction flask, 3-bromo-9H-carbazole (10 g, 49.59 mmol), 2-bromobenzen-1-ylium (a) (24.2 g, 148.77 mmol), tris(dibenzylideneacetone)dipalladium (Pd₂(dba)₃) (2.27 g, 2.48 mmol), tri-tert-butylphosphine (P(t-Bu)₃) (2.42 mL, 9.92 mmol) and sodium tert-butoxide (NatOBu) (9.53 g, 99.18 mmol) were introduced, and after introducing toluene (100 mL) thereto, the mixture was heated for 15 hours at 135° C. When the reaction was finished, the result was extracted with methylene chloride (MC) and water, and then purified by column chromatography to obtain Compound 2-1-1 (14 g, yield 98%).

### Preparation Example 6-2. Preparation of Compound 2-1

In a reaction flask, Compound 2-1-1 (14 g, 43.4 mmol), (9-phenyl-9H-carbazol-3-yl)boronic acid (b) (14.9 g, 52 mmol), tetrakis(triphenylphosphine)palladium(0) (Pd(PPh₃)₄) (2.5 g, 2.17 mmol) and potassium carbonate (K₂CO₃) (17.9 g, 130 mmol) were introduced thereto, and after adding 1,4-dioxane (140 mL) and distilled water (35 mL) thereto, the mixture was stirred for 4 hours at 120° C.

After that, the temperature was lowered to room temperature, and the produced solid was washed with distilled water and methanol to obtain Compound 2-1 (17 g, yield 80%).

Target compounds were synthesized as in the following Table 8 in the same manner as in Preparation Example 6 except that Compound a of the following Table 8 was used instead of 2-bromobenzen-1-ylium (a), and Compound b of the following Table 8 was used instead of (9-phenyl-9H-carbazol-3-yl)boronic acid (b).

TABLE 8

| Compound | | | Target Compound |
|---|---|---|---|
| No. | Compound a | Compound b | (Yield %) |
| 2-2 | 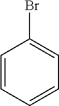 | 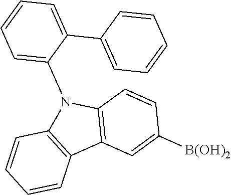 [SMILES: OB(O)c1ccc2c(c1)c1ccccc1n2-c1ccccc1-c1ccccc1] | 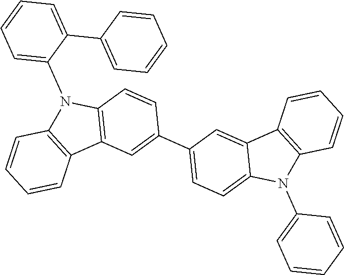 [SMILES: c1ccc(-c2ccccc2-n2c3ccccc3c3cc(-c4ccc5c(c4)c4ccccc4n5-c4ccccc4)ccc32)cc1] |
| | | | 82% |
| 2-3 | 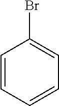 | 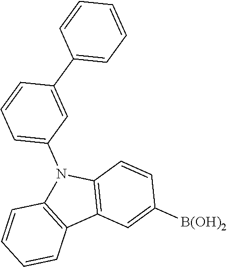 [SMILES: OB(O)c1ccc2c(c1)c1ccccc1n2-c1cccc(-c2ccccc2)c1] | 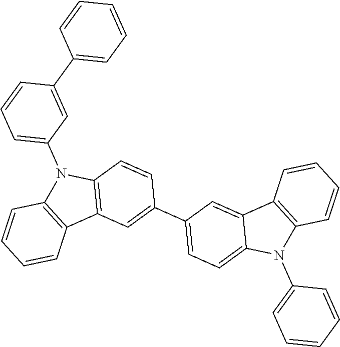 [SMILES: c1ccc(-c2cccc(-n3c4ccccc4c4cc(-c5ccc6c(c5)c5ccccc5n6-c5ccccc5)ccc43)c2)cc1] |
| | | | 80% |
| 2-4 | 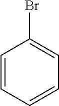 | 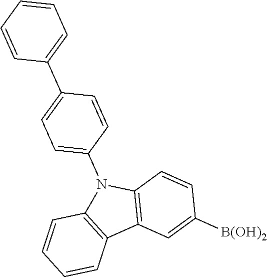 [SMILES: OB(O)c1ccc2c(c1)c1ccccc1n2-c1ccc(-c2ccccc2)cc1] | 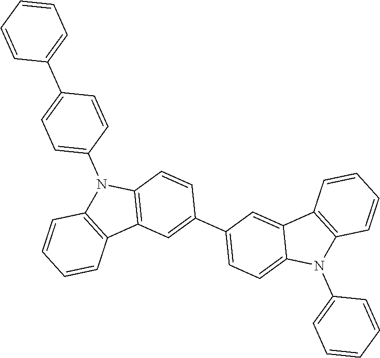 [SMILES: c1ccc(-c2ccc(-n3c4ccccc4c4cc(-c5ccc6c(c5)c5ccccc5n6-c5ccccc5)ccc43)cc2)cc1] |
| | | | 83% |
| 2-5 | 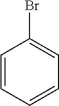 | 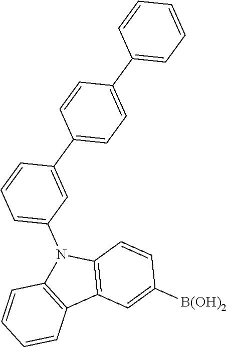 [SMILES: OB(O)c1ccc2c(c1)c1ccccc1n2-c1cccc(-c2ccc(-c3ccccc3)cc2)c1] | 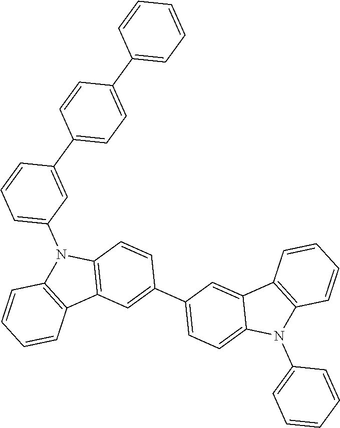 [SMILES: c1ccc(-c2ccc(-c3cccc(-n4c5ccccc5c5cc(-c6ccc7c(c6)c6ccccc6n7-c6ccccc6)ccc54)c3)cc2)cc1] |
| | | | 88% |
| 2-6 | 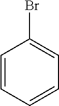 | 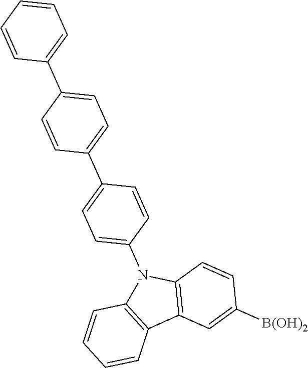 [SMILES: OB(O)c1ccc2c(c1)c1ccccc1n2-c1ccc(-c2ccc(-c3ccccc3)cc2)cc1] | 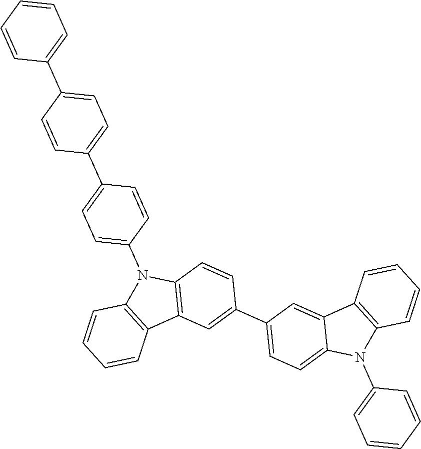 [SMILES: c1ccc(-c2ccc(-c3ccc(-n4c5ccccc5c5cc(-c6ccc7c(c6)c6ccccc6n7-c6ccccc6)ccc54)cc3)cc2)cc1] |
| | | | 86% |
| 2-7 | 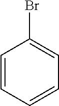 | 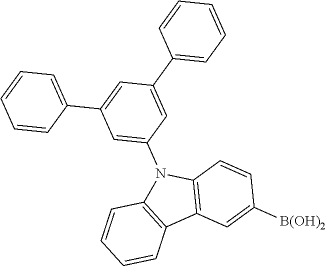 [SMILES: OB(O)c1ccc2c(c1)c1ccccc1n2-c1cc(-c2ccccc2)cc(-c2ccccc2)c1] | 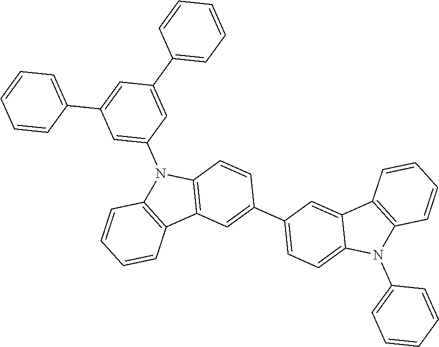 [SMILES: c1ccc(-c2cc(-c3ccccc3)cc(-n3c4ccccc4c4cc(-c5ccc6c(c5)c5ccccc5n6-c5ccccc5)ccc43)c2)cc1] |
| | | | 80% |
| 2-10 | 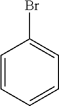 | 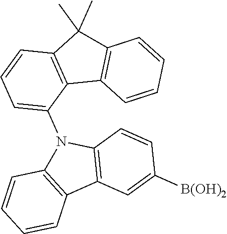 [SMILES: CC1(C)c2ccccc2-c2c(-n3c4ccccc4c4cc(B(O)O)ccc43)cccc21] | 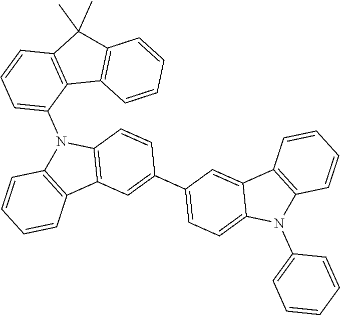 [SMILES: CC1(C)c2ccccc2-c2c(-n3c4ccccc4c4cc(-c5ccc6c(c5)c5ccccc5n6-c5ccccc5)ccc43)cccc21] |
| | | | 81% |
| 2-16 | 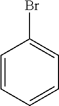 | 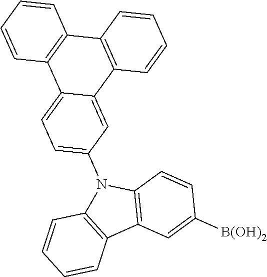 [SMILES: OB(O)c1ccc2c(c1)c1ccccc1n2-c1ccc2c3ccccc3c3ccccc3c2c1] | 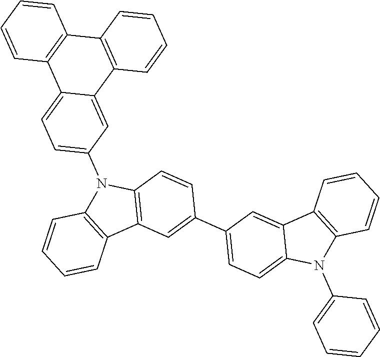 [SMILES: c1ccc(-n2c3ccccc3c3cc(-c4ccc5c(c4)c4ccccc4n5-c4ccc5c6ccccc6c6ccccc6c5c4)ccc32)cc1] |
| | | | 80% |
| 2-19 | 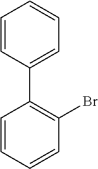 [SMILES: Brc1ccccc1-c1ccccc1] | 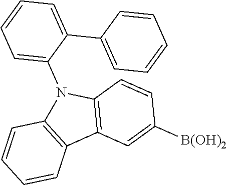 [SMILES: OB(O)c1ccc2c(c1)c1ccccc1n2-c1ccccc1-c1ccccc1] | 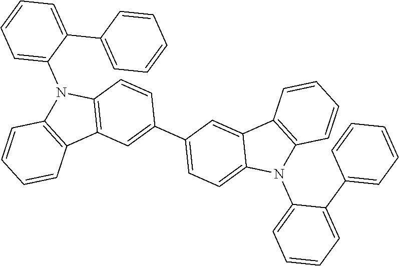 [SMILES: c1ccc(-c2ccccc2-n2c3ccccc3c3cc(-c4ccc5c(c4)c4ccccc4n5-c4ccccc4-c4ccccc4)ccc32)cc1] |
| | | | 83% |
| 2-20 | 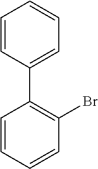 [SMILES: Brc1ccccc1-c1ccccc1] | 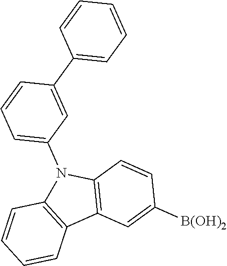 [SMILES: OB(O)c1ccc2c(c1)c1ccccc1n2-c1cccc(-c2ccccc2)c1] | 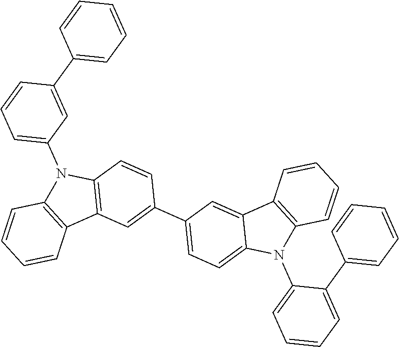 [SMILES: c1ccc(-c2cccc(-n3c4ccccc4c4cc(-c5ccc6c(c5)c5ccccc5n6-c5ccccc5-c5ccccc5)ccc43)c2)cc1] |
| | | | 81% |
| 2-21 | 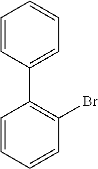 [SMILES: Brc1ccccc1-c1ccccc1] | 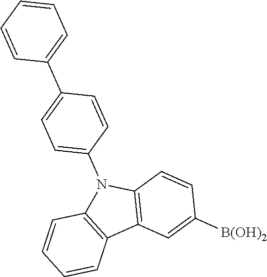 [SMILES: OB(O)c1ccc2c(c1)c1ccccc1n2-c1ccc(-c2ccccc2)cc1] | 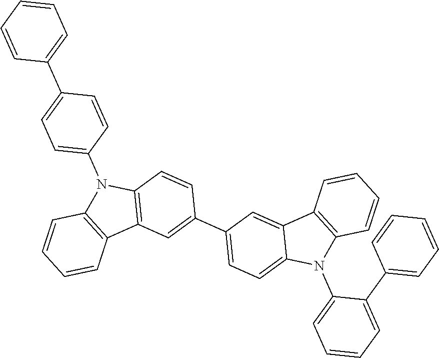 [SMILES: c1ccc(-c2ccc(-n3c4ccccc4c4cc(-c5ccc6c(c5)c5ccccc5n6-c5ccccc5-c5ccccc5)ccc43)cc2)cc1] |
| | | | 80% |
| 2-22 | 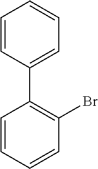 [SMILES: Brc1ccccc1-c1ccccc1] | 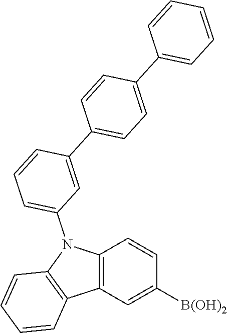 [SMILES: OB(O)c1ccc2c(c1)c1ccccc1n2-c1cccc(-c2ccc(-c3ccccc3)cc2)c1] | 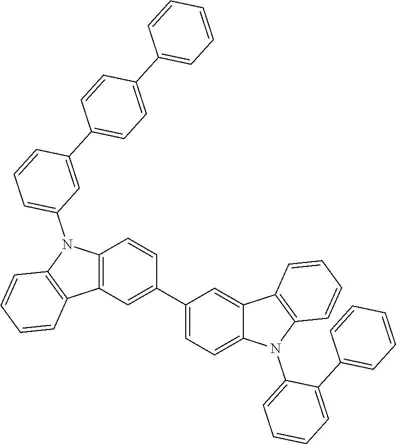 [SMILES: c1ccc(-c2ccc(-c3cccc(-n4c5ccccc5c5cc(-c6ccc7c(c6)c6ccccc6n7-c6ccccc6-c6ccccc6)ccc54)c3)cc2)cc1] |
| | | | 79% |
| 2-23 | 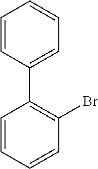 [SMILES: Brc1ccccc1-c1ccccc1] | 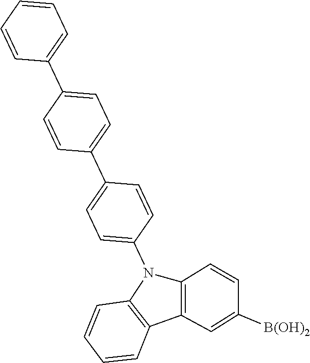 [SMILES: OB(O)c1ccc2c(c1)c1ccccc1n2-c1ccc(-c2ccc(-c3ccccc3)cc2)cc1] | 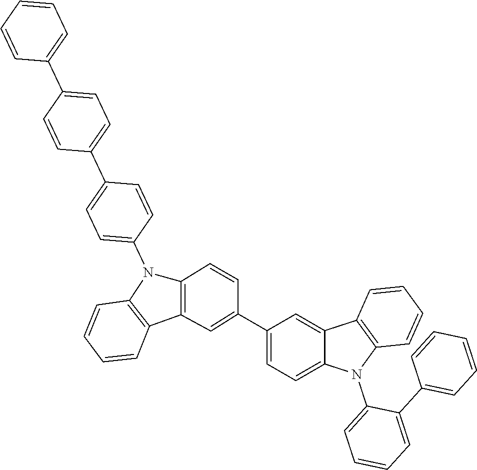 [SMILES: c1ccc(-c2ccc(-c3ccc(-n4c5ccccc5c5cc(-c6ccc7c(c6)c6ccccc6n7-c6ccccc6-c6ccccc6)ccc54)cc3)cc2)cc1] |
| | | | 82% |
| 2-26 | 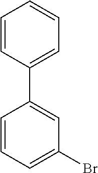 [SMILES: Brc1cccc(-c2ccccc2)c1] | 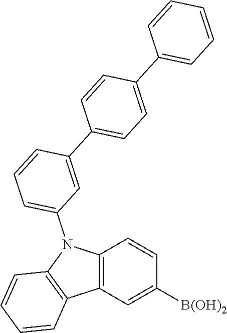 [SMILES: OB(O)c1ccc2c(c1)c1ccccc1n2-c1cccc(-c2ccc(-c3ccccc3)cc2)c1] | 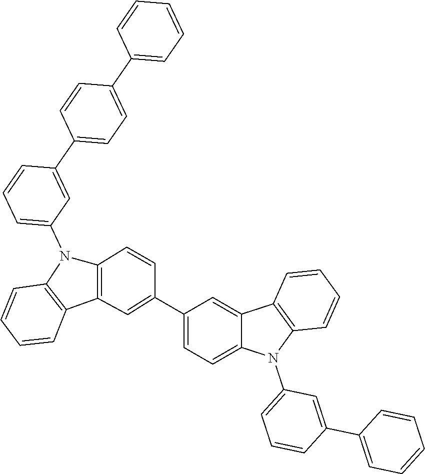 [SMILES: c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1] |
| | | | 81% |
| 2-27 | 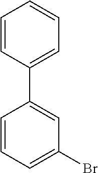 [SMILES: Brc1cccc(-c2ccccc2)c1] | 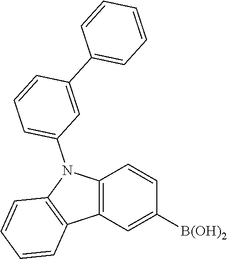 [SMILES: OB(O)c1ccc2c(c1)c1ccccc1n2-c1cccc(-c2ccccc2)c1] | 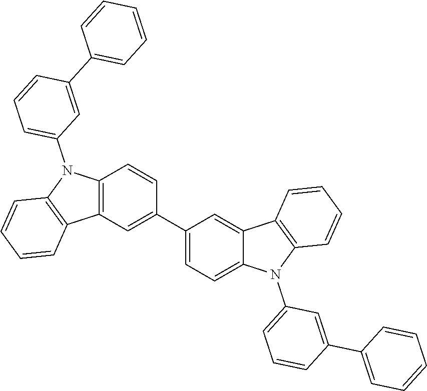 [SMILES: c1ccc(-c2cccc(-n3c4ccccc4c4cc(-c5ccc6c(c5)c5ccccc5n6-c5cccc(-c6ccccc6)c5)ccc43)c2)cc1] |
| | | | 85% |
| 2-28 | 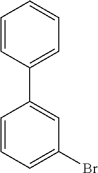 [SMILES: Brc1cccc(-c2ccccc2)c1] | 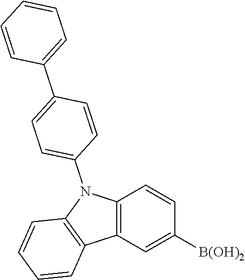 [SMILES: OB(O)c1ccc2c(c1)c1ccccc1n2-c1ccc(-c2ccccc2)cc1] | 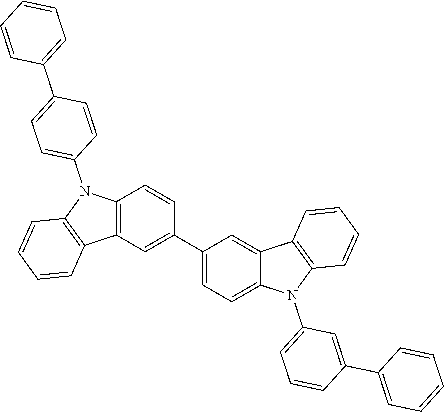 [SMILES: c1ccc(-c2ccc(-n3c4ccccc4c4cc(-c5ccc6c(c5)c5ccccc5n6-c5cccc(-c6ccccc6)c5)ccc43)cc2)cc1] |
| | | | 81% |
| 2-29 | 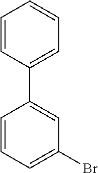 [SMILES: Brc1cccc(-c2ccccc2)c1] | 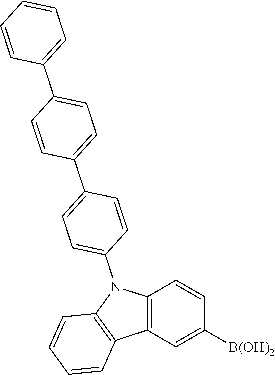 [SMILES: OB(O)c1ccc2c(c1)c1ccccc1n2-c1ccc(-c2ccc(-c3ccccc3)cc2)cc1] | 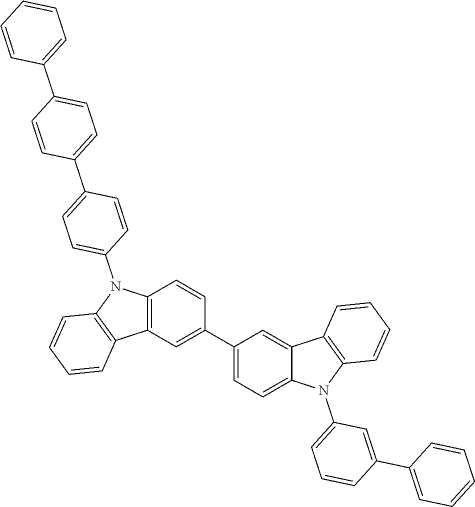 [SMILES: c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1] |
| | | | 80% |
| 2-30 | 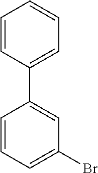 [SMILES: Brc1cccc(-c2ccccc2)c1] | 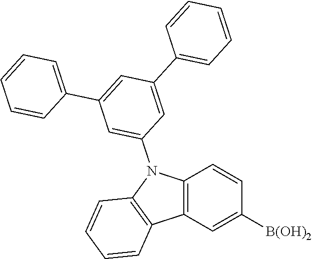 [SMILES: OB(O)c1ccc2c(c1)c1ccccc1n2-c1cc(-c2ccccc2)cc(-c2ccccc2)c1] | 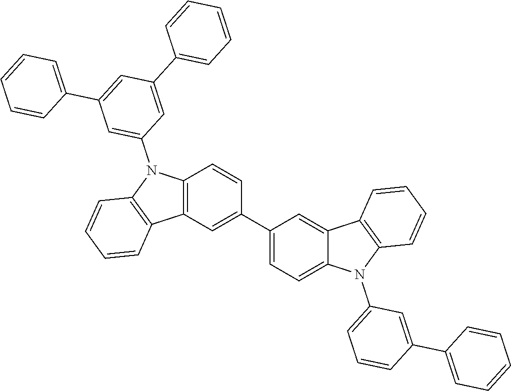 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1] |
| | | | 79% |
| 2-32 | 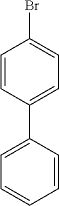 [SMILES: Brc1ccc(-c2ccccc2)cc1] | 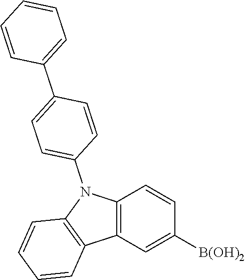 [SMILES: OB(O)c1ccc2c(c1)c1ccccc1n2-c1ccc(-c2ccccc2)cc1] | 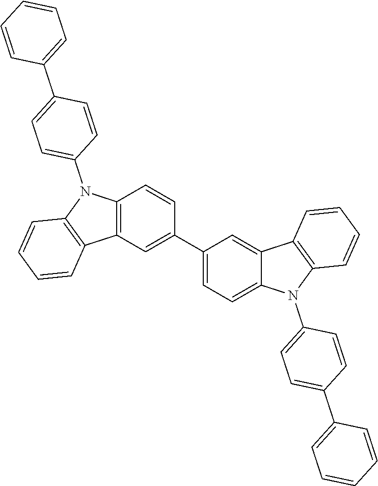 [SMILES: c1ccc(-c2ccc(-n3c4ccccc4c4cc(-c5ccc6c(c5)c5ccccc5n6-c5ccc(-c6ccccc6)cc5)ccc43)cc2)cc1] |
| | | | 81% |
| 2-33 | 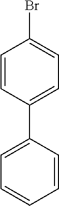 [SMILES: Brc1ccc(-c2ccccc2)cc1] | 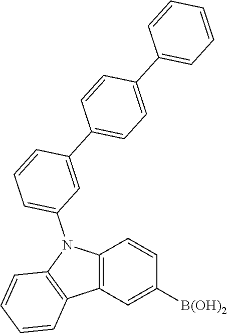 [SMILES: OB(O)c1ccc2c(c1)c1ccccc1n2-c1cccc(-c2ccc(-c3ccccc3)cc2)c1] | 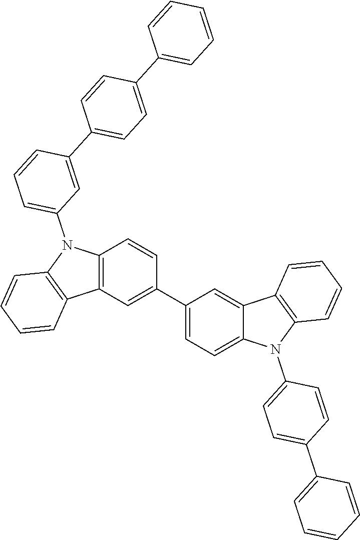 [SMILES: c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1] |
| | | | 81% |
| 2-34 | 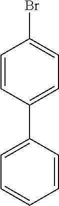 [SMILES: Brc1ccc(-c2ccccc2)cc1] | 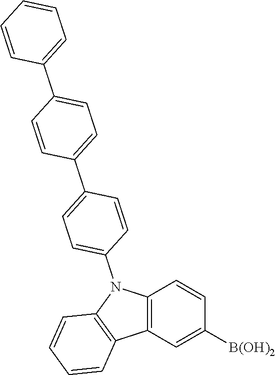 [SMILES: OB(O)c1ccc2c(c1)c1ccccc1n2-c1ccc(-c2ccc(-c3ccccc3)cc2)cc1] | 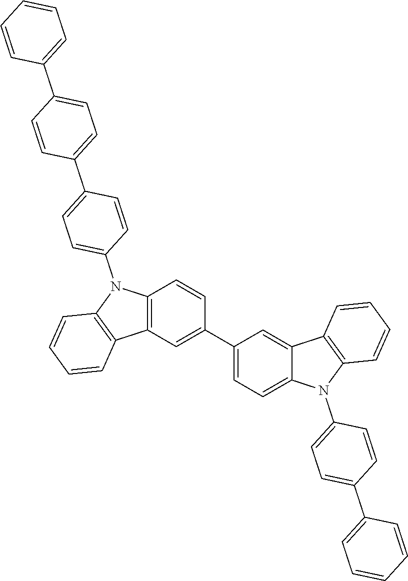 [SMILES: c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1] |
| | | | 82% |
| 2-38 | 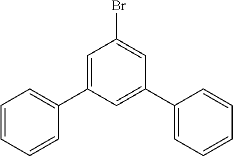 [SMILES: Brc1cc(-c2ccccc2)cc(-c2ccccc2)c1] | 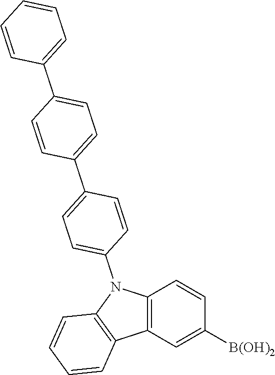 [SMILES: OB(O)c1ccc2c(c1)c1ccccc1n2-c1ccc(-c2ccc(-c3ccccc3)cc2)cc1] | 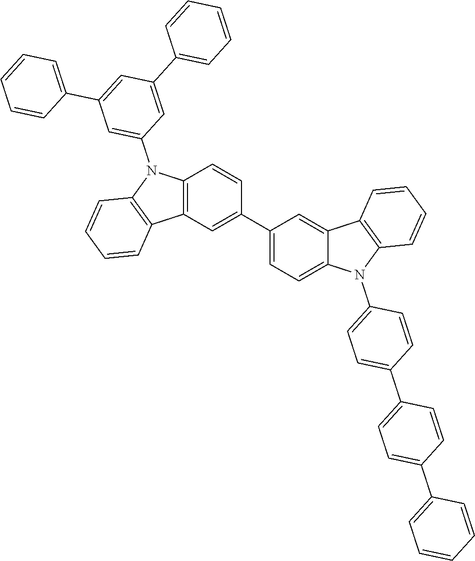 [SMILES: c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1] |
| | | | 82% |
| 2-40 | 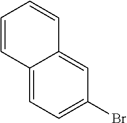 [SMILES: Brc1ccc2ccccc2c1] | 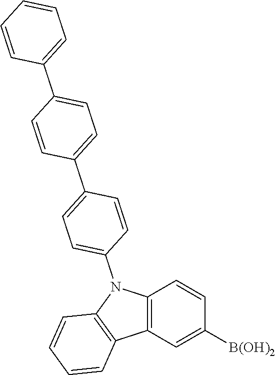 [SMILES: OB(O)c1ccc2c(c1)c1ccccc1n2-c1ccc(-c2ccc(-c3ccccc3)cc2)cc1] | 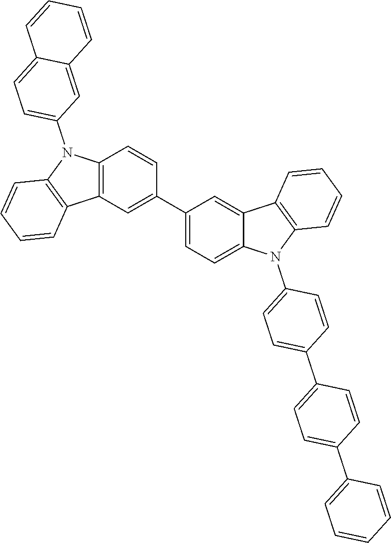 [SMILES: c1ccc(-c2ccc(-c3ccc(-n4c5ccccc5c5cc(-c6ccc7c(c6)c6ccccc6n7-c6ccc7ccccc7c6)ccc54)cc3)cc2)cc1] |
| | | | 81% |
| 2-41 | 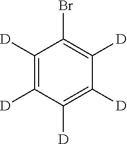 [SMILES: [2H]c1c([2H])c([2H])c(Br)c([2H])c1[2H]] | 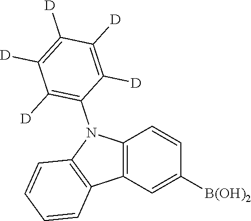 [SMILES: [2H]c1c([2H])c([2H])c(-n2c3ccccc3c3cc(B(O)O)ccc32)c([2H])c1[2H]] | 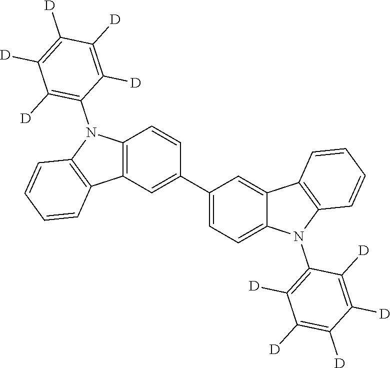 [SMILES: [2H]c1c([2H])c([2H])c(-n2c3ccccc3c3cc(-c4ccc5c(c4)c4ccccc4n5-c4c([2H])c([2H])c([2H])c([2H])c4[2H])ccc32)c([2H])c1[2H]] |
| | | | 80% |
| 2-42 | 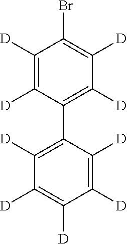 [SMILES: [2H]c1c([2H])c([2H])c(-c2c([2H])c([2H])c(Br)c([2H])c2[2H])c([2H])c1[2H]] | 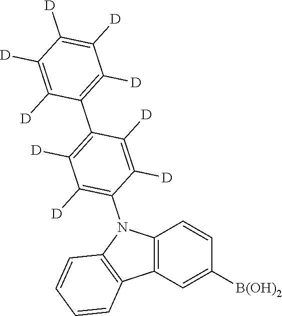 | 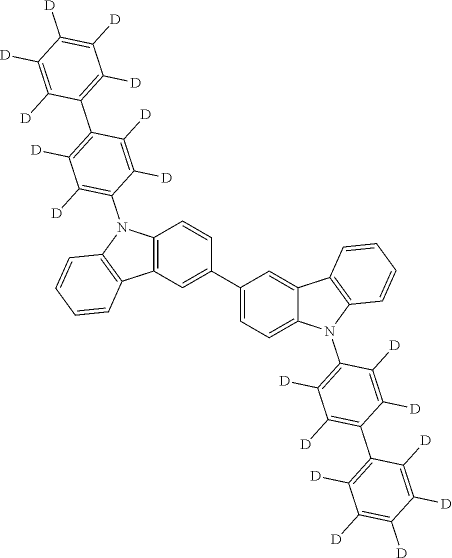 |
| | | | 79% |
| 2-43 | 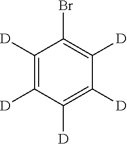 [SMILES: [2H]c1c([2H])c([2H])c(Br)c([2H])c1[2H]] | 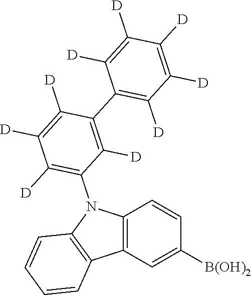 | 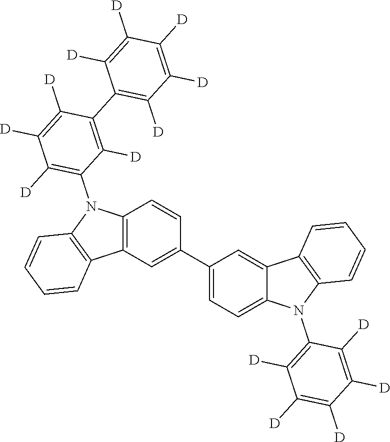 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c([2H])c([2H])c5[2H])ccc43)c2[2H])c([2H])c1[2H]] |
| | | | 78% |
| 2-45 | 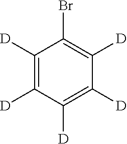 [SMILES: [2H]c1c([2H])c([2H])c(Br)c([2H])c1[2H]] | 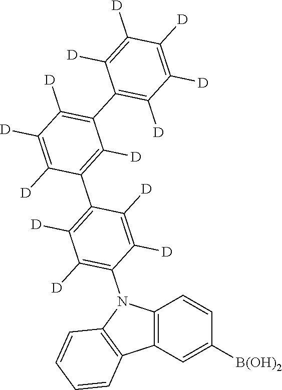 | 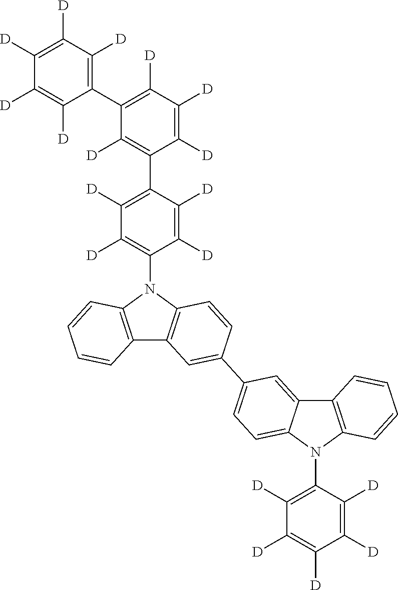 [SMILES: [2H]c1c([2H])c([2H])c(-c2c([2H])c([2H])c([2H])c(-c3c([2H])c([2H])c(-n4c5ccccc5c5cc(-c6ccc7c(c6)c6ccccc6n7-c6c([2H])c([2H])c([2H])c([2H])c6[2H])ccc54)c([2H])c3[2H])c2[2H])c([2H])c1[2H]] |
| | | | 79% |
| 2-46 | 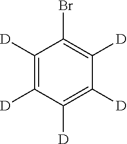 [SMILES: [2H]c1c([2H])c([2H])c(Br)c([2H])c1[2H]] | 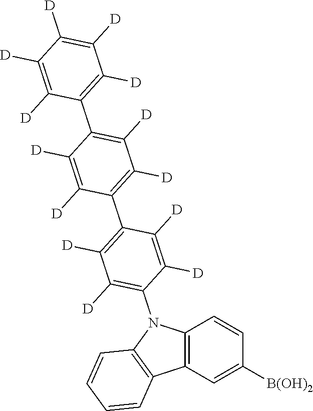 | 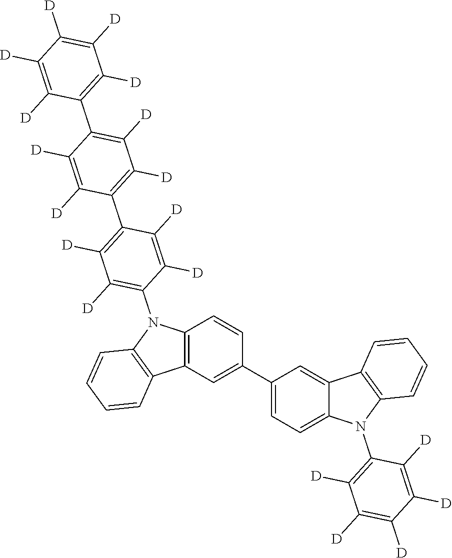 |
| | | | 80% |
| 2-48 | 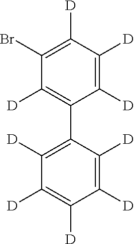 [SMILES: [2H]c1c([2H])c([2H])c(-c2c([2H])c([2H])c([2H])c(Br)c2[2H])c([2H])c1[2H]] | 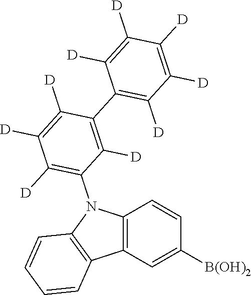 | 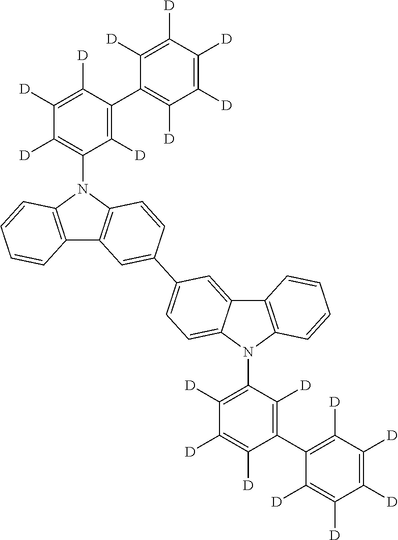 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c([2H])c(-c6c([2H])c([2H])c([2H])c([2H])c6[2H])c5[2H])ccc43)c2[2H])c([2H])c1[2H]] |
| | | | 81% |
| 2-49 | 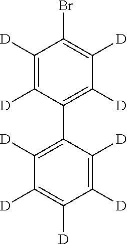 [SMILES: [2H]c1c([2H])c([2H])c(-c2c([2H])c([2H])c(Br)c([2H])c2[2H])c([2H])c1[2H]] | 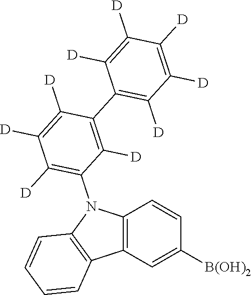 | 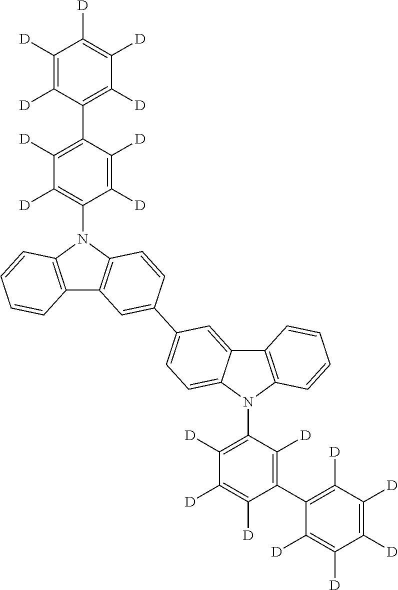 [SMILES: [2H]c1c([2H])c([2H])c(-c2c([2H])c([2H])c(-n3c4ccccc4c4cc(-c5ccc6c(c5)c5ccccc5n6-c5c([2H])c([2H])c([2H])c(-c6c([2H])c([2H])c([2H])c([2H])c6[2H])c5[2H])ccc43)c([2H])c2[2H])c([2H])c1[2H]] |
| | | | 83% |
| 2-50 | 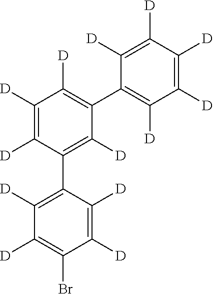 [SMILES: [2H]c1c([2H])c([2H])c(-c2c([2H])c([2H])c([2H])c(-c3c([2H])c([2H])c(Br)c([2H])c3[2H])c2[2H])c([2H])c1[2H]] | 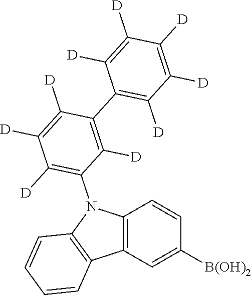 | 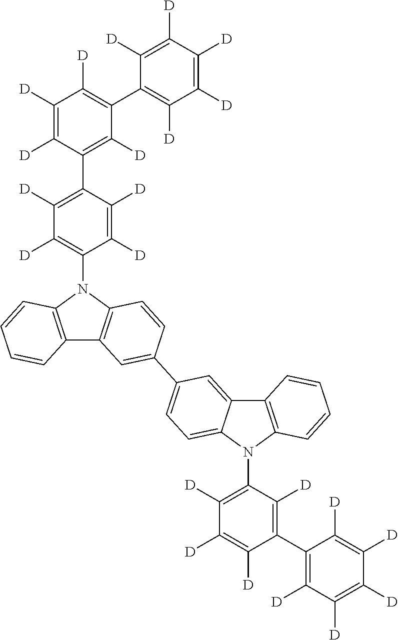 [SMILES: [2H]c1c([2H])c([2H])c(-c2c([2H])c([2H])c([2H])c(-c3c([2H])c([2H])c(-n4c5ccccc5c5cc(-c6ccc7c(c6)c6ccccc6n7-c6c([2H])c([2H])c([2H])c(-c7c([2H])c([2H])c([2H])c([2H])c7[2H])c6[2H])ccc54)c([2H])c3[2H])c2[2H])c([2H])c1[2H]] |
| | | | 82% |
| 2-51 | 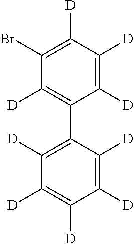 [SMILES: [2H]c1c([2H])c([2H])c(-c2c([2H])c([2H])c([2H])c(Br)c2[2H])c([2H])c1[2H]] | 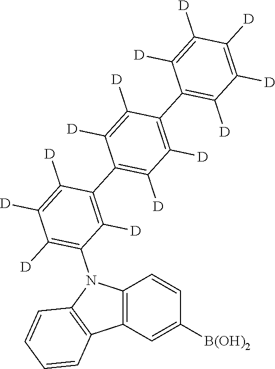 | 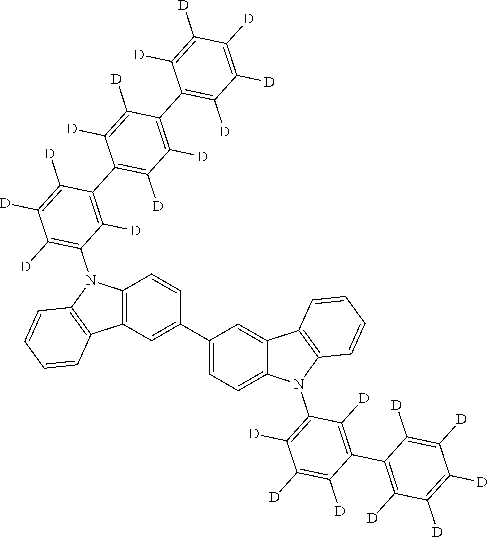 |
| | | | 84% |
| 2-52 | 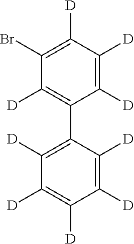 [SMILES: [2H]c1c([2H])c([2H])c(-c2c([2H])c([2H])c([2H])c(Br)c2[2H])c([2H])c1[2H]] | 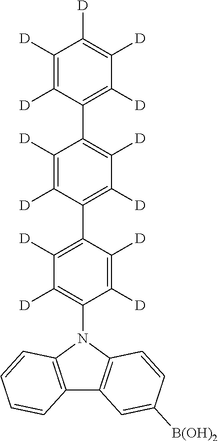 [SMILES: [2H]c1c([2H])c([2H])c(-c2c([2H])c([2H])c(-c3c([2H])c([2H])c(-n4c5ccccc5c5cc(B(O)O)ccc54)c([2H])c3[2H])c([2H])c2[2H])c([2H])c1[2H]] | 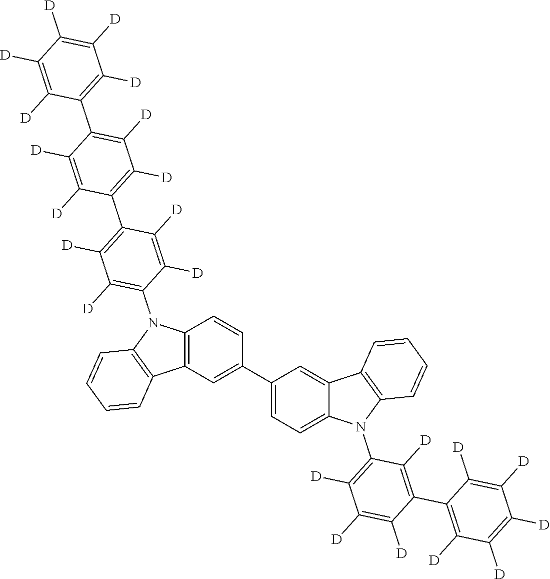 |
| | | | 80% |
| 2-55 | 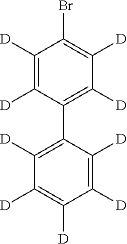 [SMILES: [2H]c1c([2H])c([2H])c(-c2c([2H])c([2H])c(Br)c([2H])c2[2H])c([2H])c1[2H]] | 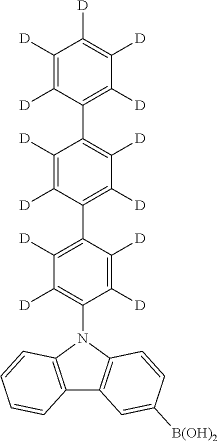 [SMILES: [2H]c1c([2H])c([2H])c(-c2c([2H])c([2H])c(-c3c([2H])c([2H])c(-n4c5ccccc5c5cc(B(O)O)ccc54)c([2H])c3[2H])c([2H])c2[2H])c([2H])c1[2H]] | 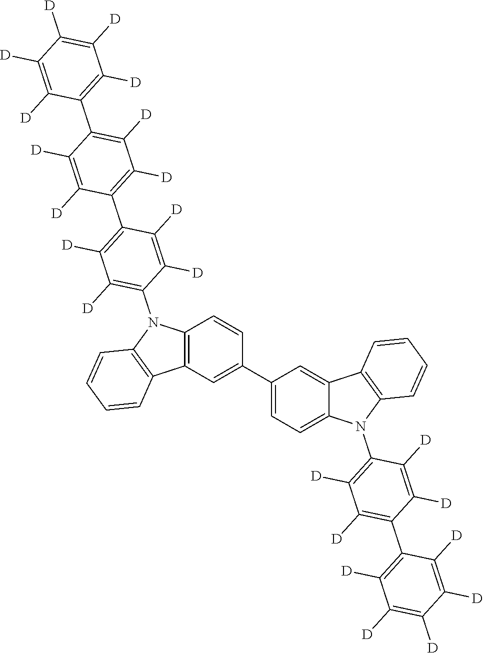 |
| | | | 81% |
| 2-57 | 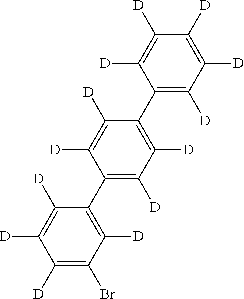 [SMILES: [2H]c1c([2H])c([2H])c(-c2c([2H])c([2H])c(-c3c([2H])c([2H])c([2H])c(Br)c3[2H])c([2H])c2[2H])c([2H])c1[2H]] | 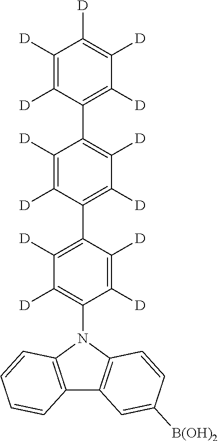 [SMILES: [2H]c1c([2H])c([2H])c(-c2c([2H])c([2H])c(-c3c([2H])c([2H])c(-n4c5ccccc5c5cc(B(O)O)ccc54)c([2H])c3[2H])c([2H])c2[2H])c([2H])c1[2H]] | 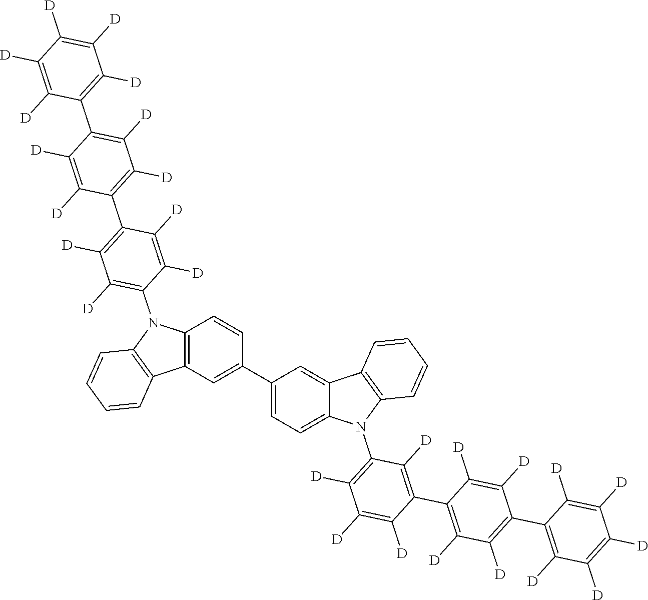 |
| | | | 82% |
| 2-60 | 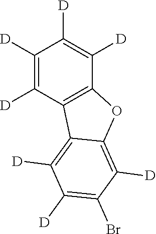 [SMILES: [2H]c1c([2H])c([2H])c2c(oc3c([2H])c(Br)c([2H])c([2H])c32)c1[2H]] | 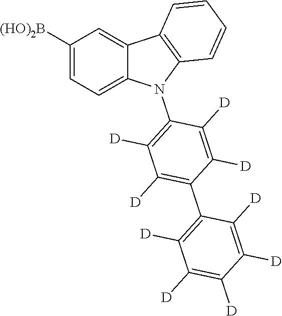 [SMILES: [2H]c1c([2H])c([2H])c(-c2c([2H])c([2H])c(-n3c4ccccc4c4cc(B(O)O)ccc43)c([2H])c2[2H])c([2H])c1[2H]] | 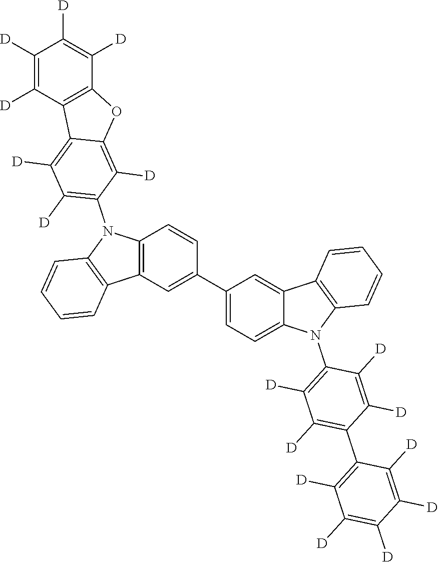 [SMILES: [2H]c1c([2H])c([2H])c(-c2c([2H])c([2H])c(-n3c4ccccc4c4cc(-c5ccc6c(c5)c5ccccc5n6-c5c([2H])c([2H])c6c(oc7c([2H])c([2H])c([2H])c([2H])c76)c5[2H])ccc43)c([2H])c2[2H])c([2H])c1[2H]] |
| | | | 82% |

### Preparation Example 7. Preparation of Compound 2-61

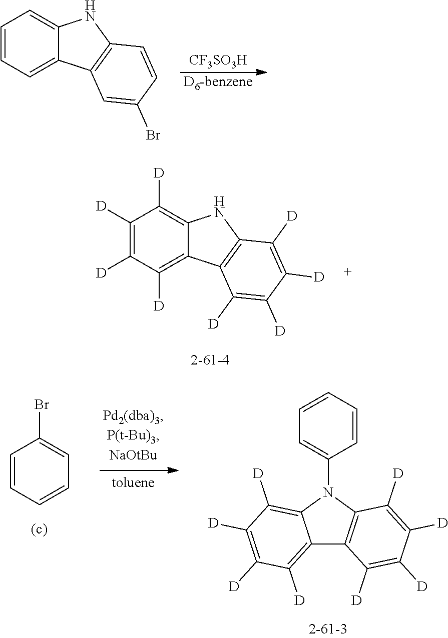

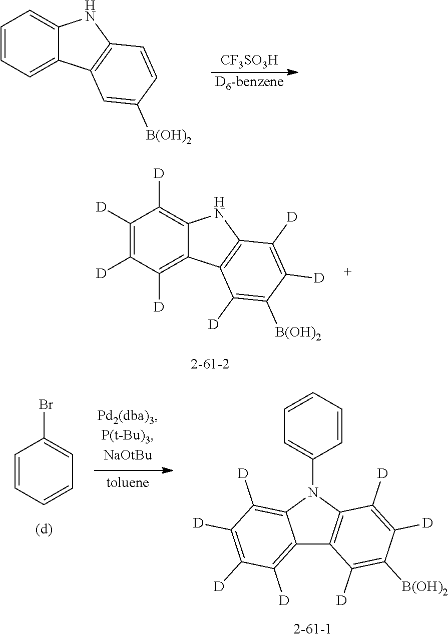

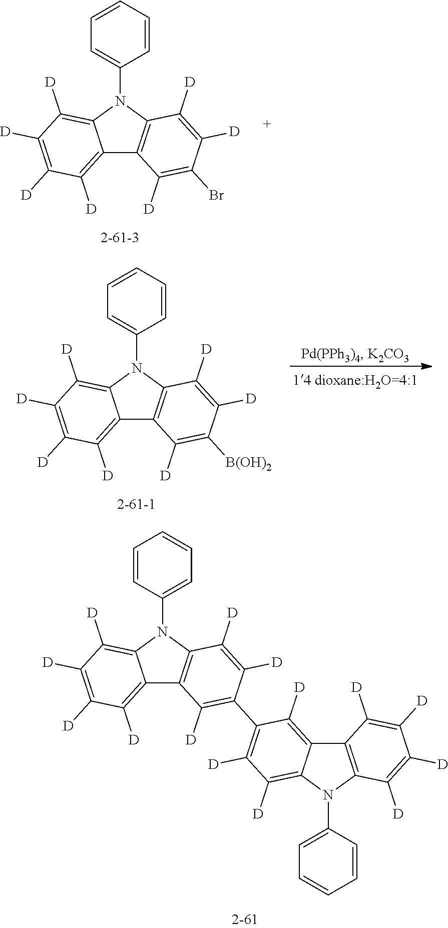

### Preparation Example 7-1. Preparation of Compound 2-61-4

3-Bromo-9H-carbazole (10 g, 40.23 mmol), D6-benzene (1000 mL) and triflic acid (CF₃SO₃H) (170 g, 1075 mmol) were introduced, and stirred at 50° C.

When the reaction was completed, the result was neutralized with D₂O and then extracted by introducing an aqueous sodium carbonate (Na₂CO₃) solution and dichloromethane (DCM) thereto at room temperature, and after drying the organic layer with anhydrous magnesium sulfate (MgSO₄), the solvent was removed using a rotary evaporator. The reaction material was purified by column chromatography (dichloromethane:hexane=1:2), and recrystallized with methanol to obtain target Compound 2-61-4 (10 g, yield 98%).

### Preparation Example 7-2. Preparation of Compound 2-61-3

Compound 2-61-4 (10 g, 39.5 mmol), bromobenzene (c) (12.4 g, 79 mmol), tris(dibenzylideneacetone) dipalladium (Pd₂(dba)₃) (1.81 g, 1.98 mmol), tri-tert-butylphosphine (P(t-Bu)₃) (1.93 mL, 7.9 mmol) and sodium tert-butoxide (NatOBu) (11.4 g, 118.51 mmol) were introduced, and after introducing toluene (100 mL) thereto, the mixture was heated to 15 hours at 135° C. When the reaction was finished, the result was extracted with methylene chloride (MC) and water, and then purified by column chromatography to obtain Compound 2-61-3 (11 g, yield 84%).

### Preparation Example 7-3. Preparation of Compound 2-61-2

9H-Carbazol-3-ylboronic acid (10 g, 47.3 mmol), D6-benzene (1000 mL) and triflic acid (CF₃SO₃H) (170 g, 1075 mmol) were introduced, and stirred at 50° C.

When the reaction was completed, the result was neutralized with D₂O and then extracted by introducing an aqueous sodium carbonate (Na₂CO₃) solution and dichloromethane (DCM) thereto at room temperature, and after drying the organic layer with anhydrous magnesium sulfate (MgSO₄), the solvent was removed using a rotary evaporator. The reaction material was purified by column chromatography (dichloromethane:hexane=1:2), and recrystallized with methanol to obtain target Compound 2-61-2 (9 g, yield 87%).

### Preparation Example 7-4. Preparation of Compound 2-61-1

Compound 2-61-2 (9 g, 41.3 mmol), bromobenzene (d) (12.9 g, 82.5 mmol), tris(dibenzylideneacetone) dipalladium (Pd₂(dba)₃) (1.89 g, 2.06 mmol), tri-tert-butylphosphine (P(t-Bu)₃) (2 mL, 8.25 mmol) and sodium tert-butoxide (NatOBu) (7.93 g, 82.574 mmol) were introduced, and after introducing toluene (100 mL) thereto, the mixture was heated for 10 hours at 135° C. When the reaction was finished, the result was extracted with methylene chloride (MC) and water, and then purified by column chromatography to obtain Compound 2-61-1 (10 g, yield 82%).

### Preparation Example 7-5. Preparation of Compound 2-61

Compound 2-61-3 (10 g, 30.37 mmol), Compound 2-61-1 (17.87 g, 60.75 mmol), tetrakis(triphenylphosphine)palladium(0) (Pd(PPh₃)₄) (1.39 g, 1.52 mmol) and potassium carbonate (K₂CO₃) (12.59 g, 91.13 mmol) were introduced, and after adding 1,4-dioxane (140 mL) and distilled water (35 mL) thereto, the mixture was stirred for 4 hours at 120° C.

After that, the temperature was lowered to room temperature, and the produced solid was washed with distilled water and methanol to obtain Compound 2-61 (13 g, yield 85%).

Target compounds were synthesized as in the following Table 9 in the same manner as in Preparation Example 7 except that Compound c of the following Table 9 was used instead of bromobenzene (c), and Compound d of the following Table 9 was used instead of bromobenzene (d).

TABLE 9

| | | | Target |
|---|---|---|---|
| Compound | | | Compound |
| No. | Compound c | Compound d | (Yield %) |
| 2-62 | 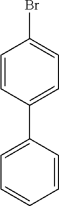 [SMILES: Brc1ccc(-c2ccccc2)cc1] | 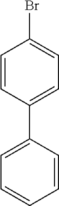 [SMILES: Brc1ccc(-c2ccccc2)cc1] | 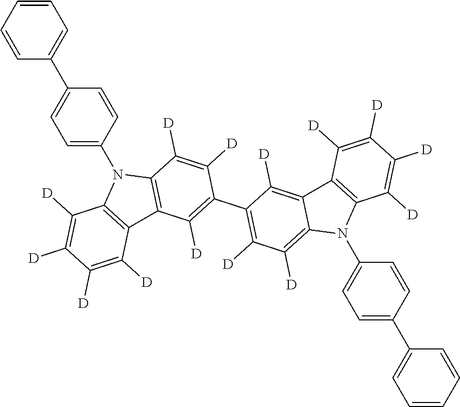 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccc(-c2ccccc2)cc1] |
| | | | 82% |
| 2-63 | 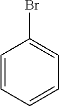 | 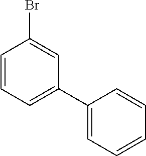 [SMILES: Brc1cccc(-c2ccccc2)c1] | 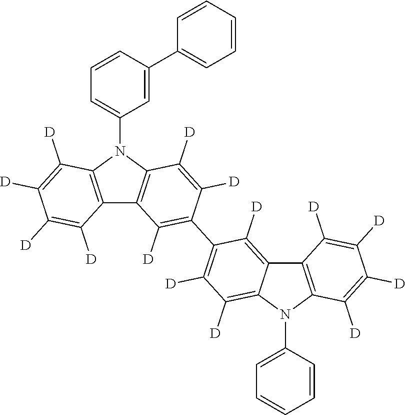 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1ccccc1] |
| | | | 84% |
| 2-64 | 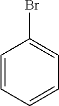 | 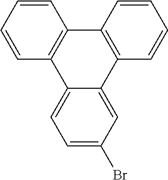 [SMILES: Brc1ccc2c3ccccc3c3ccccc3c2c1] | 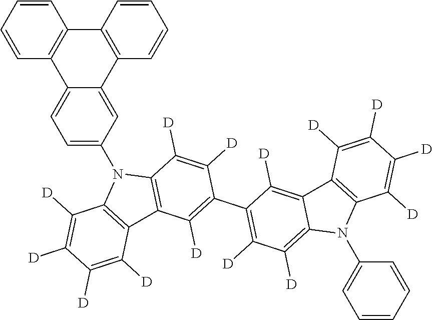 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c5ccccc5c5ccccc5c4c3)c([2H])c([2H])c1n2-c1ccccc1] |
| | | | 80% |
| 2-65 | 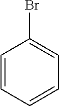 | 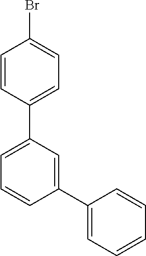 [SMILES: Brc1ccc(-c2cccc(-c3ccccc3)c2)cc1] | 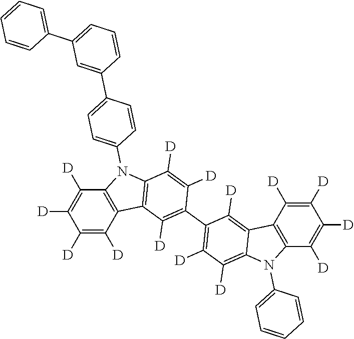 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4cccc(-c5ccccc5)c4)cc3)c([2H])c([2H])c1n2-c1ccccc1] |
| | | | 81% |
| 2-66 | 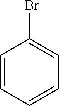 | 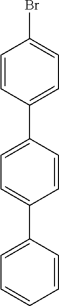 [SMILES: Brc1ccc(-c2ccc(-c3ccccc3)cc2)cc1] | 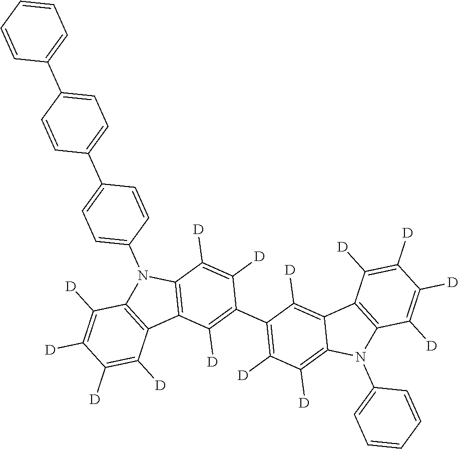 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccc(-c5ccccc5)cc4)cc3)c([2H])c([2H])c1n2-c1ccccc1] |
| | | | 82% |
| 2-68 | 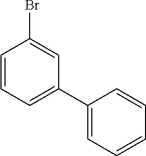 [SMILES: Brc1cccc(-c2ccccc2)c1] | 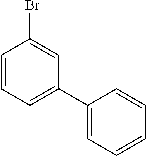 [SMILES: Brc1cccc(-c2ccccc2)c1] | 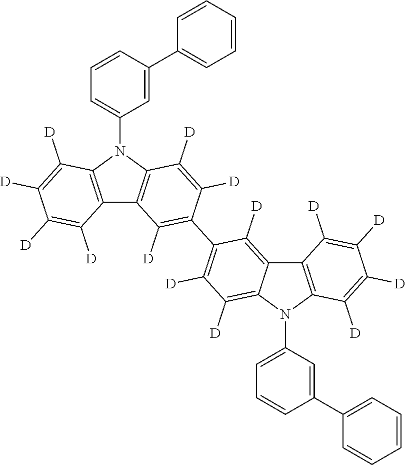 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1cccc(-c2ccccc2)c1] |
| | | | 84% |
| 2-69 | 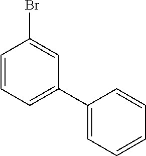 [SMILES: Brc1cccc(-c2ccccc2)c1] | 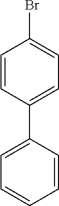 [SMILES: Brc1ccc(-c2ccccc2)cc1] | 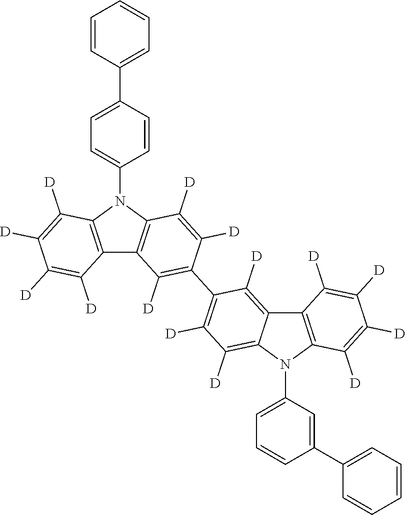 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1ccc(-c2ccccc2)cc1] |
| | | | 80% |
| 2-70 | 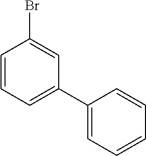 [SMILES: Brc1cccc(-c2ccccc2)c1] | 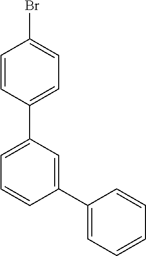 [SMILES: Brc1ccc(-c2cccc(-c3ccccc3)c2)cc1] | 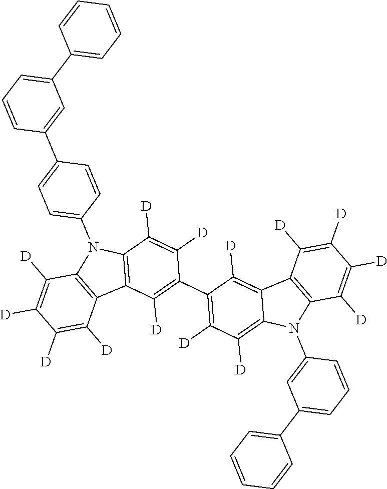 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc(-c4ccccc4)c3)c([2H])c([2H])c1n2-c1ccc(-c2cccc(-c3ccccc3)c2)cc1] |
| | | | 81% |
| 2-74 | 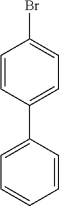 [SMILES: Brc1ccc(-c2ccccc2)cc1] | 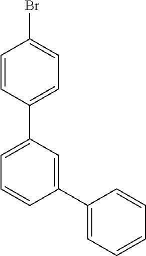 [SMILES: Brc1ccc(-c2cccc(-c3ccccc3)c2)cc1] | 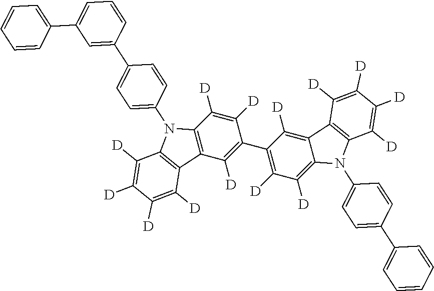 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4cccc(-c5ccccc5)c4)cc3)c([2H])c([2H])c1n2-c1ccc(-c2ccccc2)cc1] |
| | | | 81% |
| 2-75 | 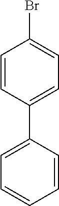 [SMILES: Brc1ccc(-c2ccccc2)cc1] | 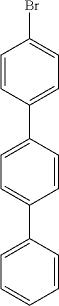 [SMILES: Brc1ccc(-c2ccc(-c3ccccc3)cc2)cc1] | 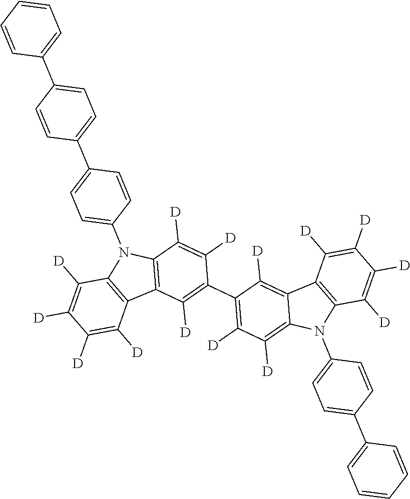 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccc(-c5ccccc5)cc4)cc3)c([2H])c([2H])c1n2-c1ccc(-c2ccccc2)cc1] |
| | | | 80% |
| 2-81 | 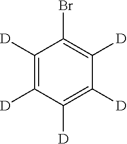 [SMILES: [2H]c1c([2H])c([2H])c(Br)c([2H])c1[2H]] | 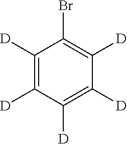 [SMILES: [2H]c1c([2H])c([2H])c(Br)c([2H])c1[2H]] | 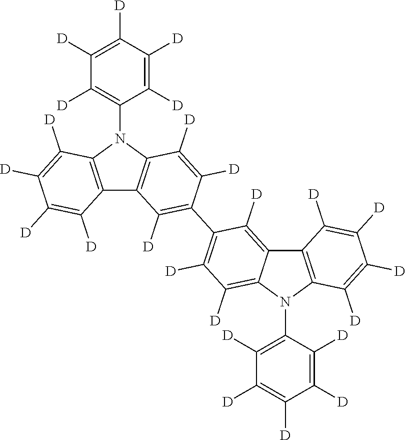 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c([2H])c4[2H])c([2H])c([2H])c32)c([2H])c1[2H]] |
| | | | 83% |
| 2-82 | 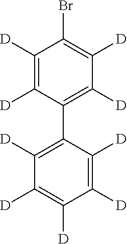 [SMILES: [2H]c1c([2H])c([2H])c(-c2c([2H])c([2H])c(Br)c([2H])c2[2H])c([2H])c1[2H]] | 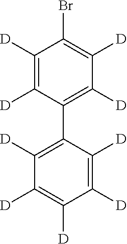 [SMILES: [2H]c1c([2H])c([2H])c(-c2c([2H])c([2H])c(Br)c([2H])c2[2H])c([2H])c1[2H]] | 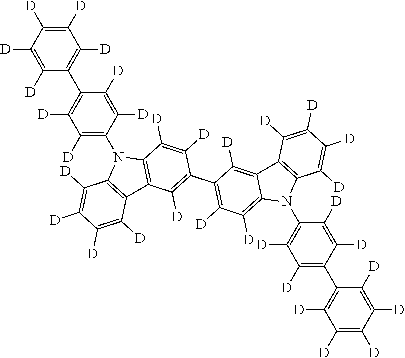 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c(-c6c([2H])c([2H])c([2H])c([2H])c6[2H])c([2H])c5[2H])c([2H])c([2H])c43)c([2H])c2[2H])c([2H])c1[2H]] |
| | | | 82% |
| 2-83 | 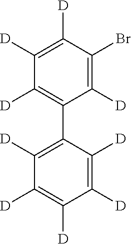 [SMILES: [2H]c1c([2H])c([2H])c(-c2c([2H])c([2H])c([2H])c(Br)c2[2H])c([2H])c1[2H]] | 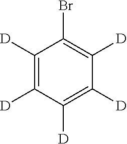 [SMILES: [2H]c1c([2H])c([2H])c(Br)c([2H])c1[2H]] | 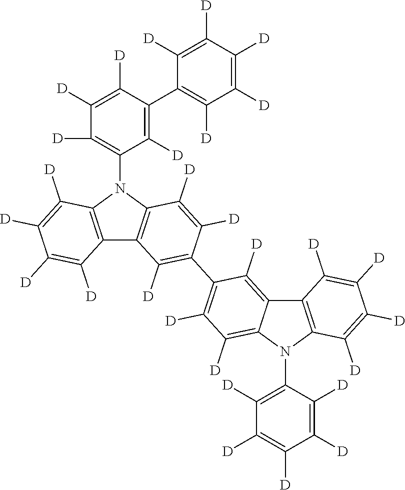 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c2[2H])c([2H])c1[2H]] |
| | | | 84% |
| 2-85 | 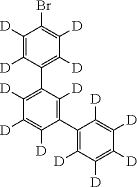 [SMILES: [2H]c1c([2H])c([2H])c(-c2c([2H])c([2H])c([2H])c(-c3c([2H])c([2H])c(Br)c([2H])c3[2H])c2[2H])c([2H])c1[2H]] | 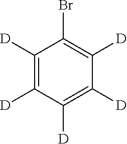 [SMILES: [2H]c1c([2H])c([2H])c(Br)c([2H])c1[2H]] | 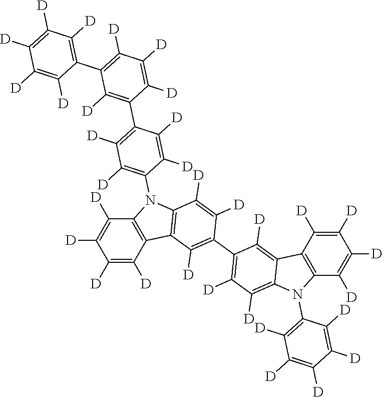 [SMILES: [2H]c1c([2H])c([2H])c(-c2c([2H])c([2H])c([2H])c(-c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c([2H])c6[2H])c([2H])c([2H])c54)c([2H])c3[2H])c2[2H])c([2H])c1[2H]] |
| | | | 80% |
| 2-86 | 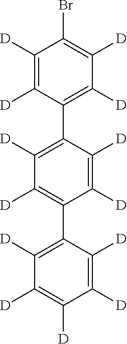 [SMILES: [2H]c1c([2H])c([2H])c(-c2c([2H])c([2H])c(-c3c([2H])c([2H])c(Br)c([2H])c3[2H])c([2H])c2[2H])c([2H])c1[2H]] | 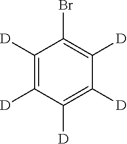 [SMILES: [2H]c1c([2H])c([2H])c(Br)c([2H])c1[2H]] | 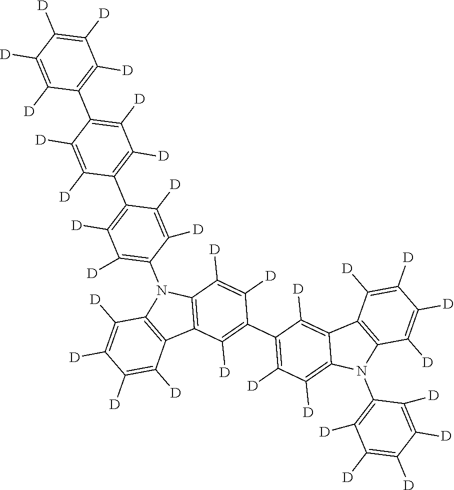 [SMILES: [2H]c1c([2H])c([2H])c(-c2c([2H])c([2H])c(-c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c([2H])c6[2H])c([2H])c([2H])c54)c([2H])c3[2H])c([2H])c2[2H])c([2H])c1[2H]] |
| | | | 81% |
| 2-87 | 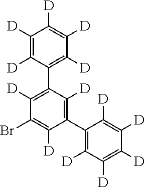 [SMILES: [2H]c1c([2H])c([2H])c(-c2c([2H])c(Br)c([2H])c(-c3c([2H])c([2H])c([2H])c([2H])c3[2H])c2[2H])c([2H])c1[2H]] | 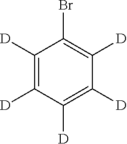 [SMILES: [2H]c1c([2H])c([2H])c(Br)c([2H])c1[2H]] | 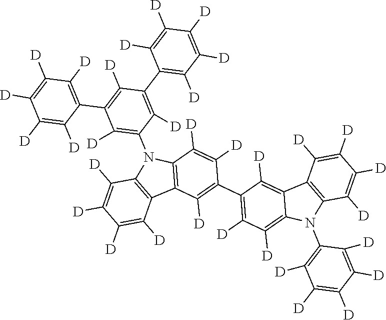 [SMILES: [2H]c1c([2H])c([2H])c(-c2c([2H])c(-c3c([2H])c([2H])c([2H])c([2H])c3[2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c2[2H])c([2H])c1[2H]] |
| | | | 81% |
| 2-88 | 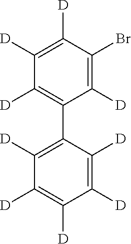 [SMILES: [2H]c1c([2H])c([2H])c(-c2c([2H])c([2H])c([2H])c(Br)c2[2H])c([2H])c1[2H]] | 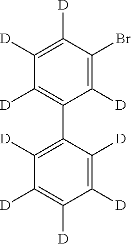 [SMILES: [2H]c1c([2H])c([2H])c(-c2c([2H])c([2H])c([2H])c(Br)c2[2H])c([2H])c1[2H]] | 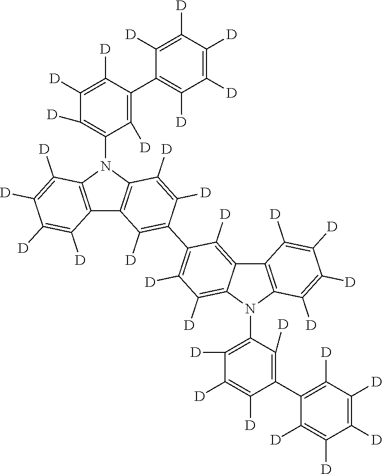 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c(-c6c([2H])c([2H])c([2H])c([2H])c6[2H])c5[2H])c([2H])c([2H])c43)c2[2H])c([2H])c1[2H]] |
| | | | 82% |
| 2-89 | 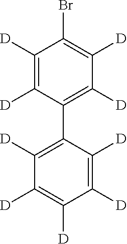 [SMILES: [2H]c1c([2H])c([2H])c(-c2c([2H])c([2H])c(Br)c([2H])c2[2H])c([2H])c1[2H]] | 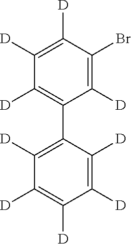 [SMILES: [2H]c1c([2H])c([2H])c(-c2c([2H])c([2H])c([2H])c(Br)c2[2H])c([2H])c1[2H]] | 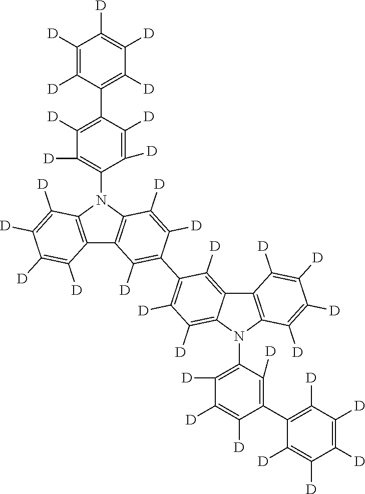 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c(-c6c([2H])c([2H])c([2H])c([2H])c6[2H])c5[2H])c([2H])c([2H])c43)c([2H])c2[2H])c([2H])c1[2H]] |
| | | | 82% |
| 2-90 | 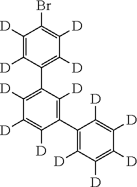 [SMILES: [2H]c1c([2H])c([2H])c(-c2c([2H])c([2H])c([2H])c(-c3c([2H])c([2H])c(Br)c([2H])c3[2H])c2[2H])c([2H])c1[2H]] | 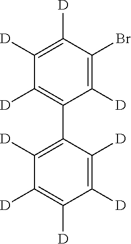 [SMILES: [2H]c1c([2H])c([2H])c(-c2c([2H])c([2H])c([2H])c(Br)c2[2H])c([2H])c1[2H]] | 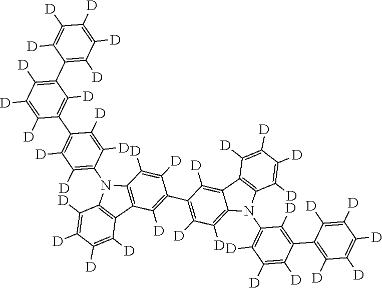 [SMILES: [2H]c1c([2H])c([2H])c(-c2c([2H])c([2H])c([2H])c(-c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c(-c7c([2H])c([2H])c([2H])c([2H])c7[2H])c6[2H])c([2H])c([2H])c54)c([2H])c3[2H])c2[2H])c([2H])c1[2H]] |
| | | | 84% |
| 2-92 | 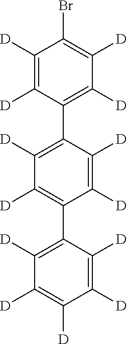 [SMILES: [2H]c1c([2H])c([2H])c(-c2c([2H])c([2H])c(-c3c([2H])c([2H])c(Br)c([2H])c3[2H])c([2H])c2[2H])c([2H])c1[2H]] | 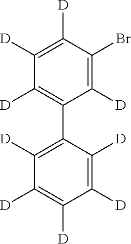 [SMILES: [2H]c1c([2H])c([2H])c(-c2c([2H])c([2H])c([2H])c(Br)c2[2H])c([2H])c1[2H]] | 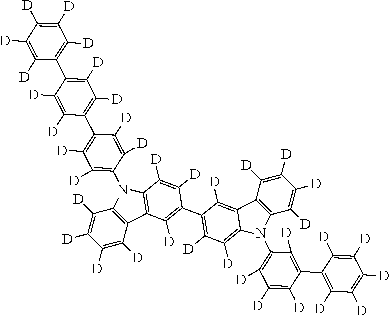 [SMILES: [2H]c1c([2H])c([2H])c(-c2c([2H])c([2H])c(-c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c(-c7c([2H])c([2H])c([2H])c([2H])c7[2H])c6[2H])c([2H])c([2H])c54)c([2H])c3[2H])c([2H])c2[2H])c([2H])c1[2H]] |
| | | | 80% |
| 2-100 | 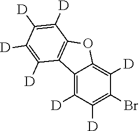 [SMILES: [2H]c1c([2H])c([2H])c2c(oc3c([2H])c(Br)c([2H])c([2H])c32)c1[2H]] | 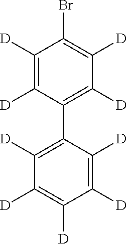 [SMILES: [2H]c1c([2H])c([2H])c(-c2c([2H])c([2H])c(Br)c([2H])c2[2H])c([2H])c1[2H]] | 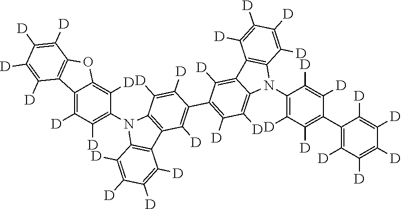 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6c(oc7c([2H])c([2H])c([2H])c([2H])c76)c5[2H])c([2H])c([2H])c43)c([2H])c2[2H])c([2H])c1[2H]] |
| | | | 84% |
| 2-102 | 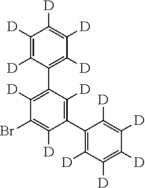 [SMILES: [2H]c1c([2H])c([2H])c(-c2c([2H])c(Br)c([2H])c(-c3c([2H])c([2H])c([2H])c([2H])c3[2H])c2[2H])c([2H])c1[2H]] | 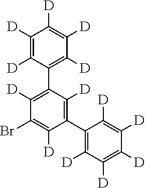 [SMILES: [2H]c1c([2H])c([2H])c(-c2c([2H])c(Br)c([2H])c(-c3c([2H])c([2H])c([2H])c([2H])c3[2H])c2[2H])c([2H])c1[2H]] | 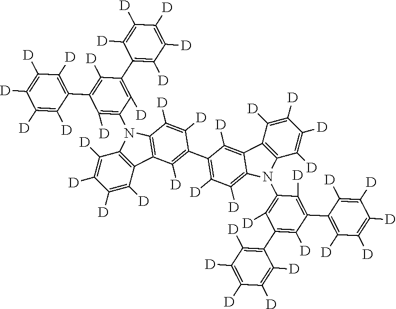 [SMILES: [2H]c1c([2H])c([2H])c(-c2c([2H])c(-c3c([2H])c([2H])c([2H])c([2H])c3[2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c(-c6c([2H])c([2H])c([2H])c([2H])c6[2H])c([2H])c(-c6c([2H])c([2H])c([2H])c([2H])c6[2H])c5[2H])c([2H])c([2H])c43)c2[2H])c([2H])c1[2H]] |
| | | | 81% |

### Preparation Example 8. Preparation of Compound 2-82

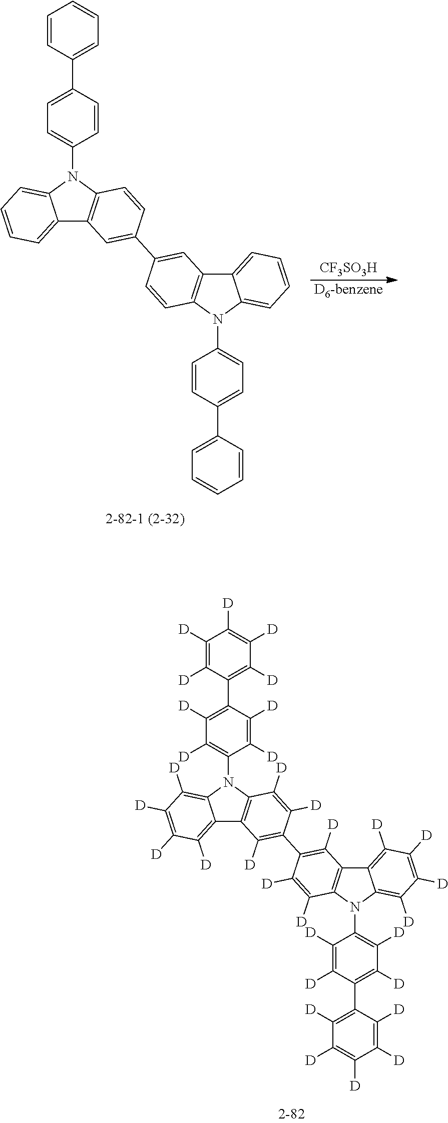

Compound 2-82-1 (Compound 2-32) (10 g, 15.7 mmol), D₆-benzene (1000 mL) and triflic acid (CF₃SO₃H) (170 g, 1075 mmol) were introduced, and stirred at 50° C.

When the reaction was completed, the result was neutralized with D20 and then extracted by introducing an aqueous sodium carbonate (Na₂CO₃) solution and dichloromethane (DCM) thereto at room temperature, and after drying the organic layer with anhydrous magnesium sulfate (MgSO₄), the solvent was removed using a rotary evaporator. The reaction material was purified by column chromatography (dichloromethane:hexane=1:2), and recrystallized with methanol to obtain target Compound 2-82 (10.0 g, yield 95%).

Synthesis results for the compounds described in Preparation Example 6 to Preparation Example 8, and Table 8 and Table 9, and synthesis results for the heterocyclic compounds corresponding to Chemical Formula 10 are shown in the following Table 10 and Table 11. The following Table 10 shows measurement values of ¹H NMR (CDCl₃, 300 MHz), and the following Table 11 shows measurement values of FD-mass spectrometry (FD-MS: field desorption mass spectrometry).

TABLE 10

| Compound No. | ¹H NMR (CDCl₃, 300 MHz) |
|---|---|
| 2-1 | δ = 8.55 (d, 1H), 8.30 (d, 1H), 8.19-8.13 (m, 2H), 7.99-7.89 (m, 4H), |
| | 7.77 (d, 1H), 7.62-7.50 (m, 12H), 7.35 (t, 1H), 7.20-7.16 (m, 2H) |
| 2-2 | δ = 8.55 (d, 1H), 8.30 (d, 1H), 8.19-8.13 (m, 2H), 7.99-7.89 (m, 6H), |
| | 7.80-7.77 (m, 2H), 7.62-7.35 (m, 10H), 7.20-7.16 (m, 6H) |
| 2-3 | δ = 8.55 (d, 1H), 8.18-8.09 (m, 3H), 8.00-7.87 (m, 3H), 7.77 (s, 2H), |
| | 7.58-7.25 (m, 18H) |
| 2-4 | δ = 8.55 (d, 1H), 8.18-8.12 (m, 2H), 8.00-7.84 (m, 3H), 7.79-7.77 (m, |
| | 4H), 7.68-7.25 (m, 22H) |
| 2-5 | δ = 8.55 (d, 1H), 8.30 (d, 1H), 8.21-8.13 (m, 3H), 7.99-7.89 (m, 4H), |
| | 7.77-7.35 (m, 17H), 7.25-7.16 (m, 6H) |
| 2-6 | δ = 8.55 (d, 1H), 8.30 (d, 1H), 8.19-8.13 (m, 2H), 7.94-7.89 (m, 8H), |
| | 7.77-7.75 (m, 3H), 7.62-7.35 (m, 11H), 7.25-7.16 (m, 6H) |
| 2-7 | δ = 8.55 (d, 1H), 8.18-8.09 (m, 4H), 8.00-7.94 (m, 2H), 7.87 (m, 1H), |
| | 7.77 (m, 2H), 7.69-7.63 (m, 2H), 7.52-7.25 (m, 20H) |
| 2-10 | δ = 8.55 (d, 1H), 8.30 (d, 1H), 8.19-8.13 (m, 2H), 7.99-7.89 (m, 5H), |
| | 7.77 (d, 1H), 7.58-7.28 (m, 16H), 1.69 (s, 6H) |
| 2-16 | δ = 9.05 (s, 1H), 8.55 (d, 1H), 8.33-8.13 (m, 7H), 7.99-7.89 (m, 5H), |
| | 7.77-7.50 (m, 13H), 7.35 (t, 1H), 7.20-7.16 (m, 2H) |
| 2-19 | δ = 8.55 (d, 1H), 8.30 (d, 1H), 8.19-8.13 (m, 2H), 7.99-7.89 (m, 8H), |
| | 7.80-7.77 (m, 3H), 7.58 (d, 1H), 7.50-7.35 (m, 6H), 7.20-7.16 (m, 10H) |
| 2-20 | δ = 8.55 (d, 1H), 8.30 (d, 1H), 8.21-8.13 (m, 3H), 7.99-7.89 (m, 6H), |
| | 7.80-7.35 (m, 15H), 7.20-7.16 (6H) |
| 2-21 | δ = 8.55 (d, 1H), 8.30 (d, 1H), 8.19-8.13 (m, 2h), 7.99-7.89 (m, 10H), |
| | 7.80-7.75 (m, 4H), 7.50-7.35 (m, 8H), 7.20-7.16 (m, 6H) |
| 2-22 | δ = 8.55 (d, 1H), 8.30 (d, 1H), 8.21-8.13 (m, 3H), 7.99-7.89 (m, 6H), |
| | 7.80-7.35 (m, 15H), 7.25-7.16 (10H) |
| 2-23 | δ = 8.55 (d, 1H), 8.30 (d, 1H), 8.19-8.13 (m, 2h), 7.99-7.89 (m, 10H), |
| | 7.80-7.75 (m, 4H), 7.50-7.35 (m, 8H), 7.25-7.16 (m, 10H) |
| 2-26 | δ = 8.55 (m, 1H), 8.30 (d, 1H), 8.21-8.13 (m, 4h), 7.99-7.89 (m, 4H), |
| | 7.77-7.35 (m, 20H), 7.25-7.16 (6H) |
| 2-27 | δ = 8.55 (m, 1H), 8.30 (d, 1H), 8.21-8.13 (m, 4h), 7.99-7.89 (m, 4H), |
| | 7.77-7.35 (m, 20H), 7.20-7.16 (2H) |
| 2-28 | δ = 8.55 (m, 1H), 8.18-8.09 (m, 3H), 8.00-8.79 (m, 2H), 7.87 (m, 1H), |
| | 7.79-7.77 (m, 4H), 7.69-7.63 (m, 4H), 7.52-7.25 (m, 12H) |
| 2-29 | δ = 8.55 (m, 1H), 8.18-8.09 (m, 3H), 8.00-7.94 (m, 2H0, 7.87 (m, 1H), |
| | 7.87 (m, 1H), 7.79-7.77 (m, 4H), 7.69-7.63 (m, 4H), 7.52-7.25 (m, 21H) |
| 2-30 | δ = 8.55 (m, 1H), 8.31-8.30 (m, 3H), 8.21-8.13 (m, 3h), 7.99-7.89 (m, |
| | 3H), 7.75-7.35 (m, 22H), 7.20-7.16 (m, 2H) |
| 2-32 | δ = 8.55 (m, 1H), 8.18-8.12 (m, 2H), 8.00-7.87 (m, 3H), 7.79-7.77 (m, |
| | 6H), 7.69-7.63 (m, 6H), 7.52-7.25 (m, 14H) |
| 2-33 | δ = 8.55 (m, 1H), 8.30 (d, 1H), 8.21-8.13 (m, 3H), 7.99-7.89 (m, 8H), |
| | 7.77-7.35 (m, 17H), 7.25-7.16 (6H) |
| 2-34 | δ = 8.55 (m, 1H), 8.18-8.12 (m, 2H), 8.00-7.87 (m, 3H), 7.79-7.77 (m, |
| | 6H), 7.67-7.63 (m, 6H), 7.52-7.25 (m, 18H) |
| 2-38 | δ = 8.55 (m, 1H), 8.18-8.12 (m, 2H), 8.05-7.87 (m, 6H), 7.79-7.77 (m, |
| | 4H), 7.69-7.63 (m, 4H), 7.52-7.25 (m, 23H) |
| 2-40 | δ = 8.55 (m, 1H), 8.30 (d, 1H), 8.19-8.13 (m, 2H), 8.03-7.75 (m, 15H), |
| | 7.58-7.35 (m, 9H), 7.25-7.16 (m, 6H) |
| 2-41 | δ = 8.55 (m, 1H), 8.30 (d, 1H), 8.19-8.13 (m, 2H), 7.99-7.89 (m, 4H), |
| | 7.77 (d, 1H), 7.58-7.50 (m, 2H), 7.35 (t, 1H), 7.20-7.16 (m, 2H) |
| 2-42 | δ = 8.55 (m, 1H), 8.30 (d, 1H), 8.19-8.13 (m, 2H), 7.99-7.89 (m, 4H), |
| | 7.77 (d, 1H), 7.58-7.50 (m, 2H), 7.35 (t, 1H), 7.20-7.16 (m, 2H) |
| 2-43 | δ = 8.55 (m, 1H), 8.30 (d, 1H), 8.19-8.13 (m, 2H), 7.99-7.89 (m, 4H), |
| | 7.77 (d, 1H), 7.58-7.50 (m, 2H), 7.35 (t, 1H), 7.20-7.16 (m, 2H) |
| 2-45 | δ = 8.55 (m, 1H), 8.30 (d, 1H), 8.19-8.13 (m, 2H), 7.99-7.89 (m, 4H), |
| | 7.77 (d, 1H), 7.58-7.50 (m, 2H), 7.35 (t, 1H), 7.20-7.16 (m, 2H) |
| 2-46 | δ = 8.55 (m, 1H), 8.30 (d, 1H), 8.19-8.13 (m, 2H), 7.99-7.89 (m, 4H), |
| | 7.77 (d, 1H), 7.58-7.50 (m, 2H), 7.35 (t, 1H), 7.20-7.16 (m, 2H) |
| 2-48 | δ = 8.55 (m, 1H), 8.30 (d, 1H), 8.19-8.13 (m, 2H), 7.99-7.89 (m, 4H), |
| | 7.77 (d, 1H), 7.58-7.50 (m, 2H), 7.35 (t, 1H), 7.20-7.16 (m, 2H) |
| 2-49 | δ = 8.55 (m, 1H), 8.30 (d, 1H), 8.19-8.13 (m, 2H), 7.99-7.89 (m, 4H), |
| | 7.77 (d, 1H), 7.58-7.50 (m, 2H), 7.35 (t, 1H), 7.20-7.16 (m, 2H) |
| 2-50 | δ = 8.55 (m, 1H), 8.30 (d, 1H), 8.19-8.13 (m, 2H), 7.99-7.89 (m, 4H), |
| | 7.77 (d, 1H), 7.58-7.50 (m, 2H), 7.35 (t, 1H), 7.20-7.16 (m, 2H) |
| 2-51 | δ = 8.55 (m, 1H), 8.30 (d, 1H), 8.19-8.13 (m, 2H), 7.99-7.89 (m, 4H), |
| | 7.77 (d, 1H), 7.58-7.50 (m, 2H), 7.35 (t, 1H), 7.20-7.16 (m, 2H) |
| 2-52 | δ = 8.55 (m, 1H), 8.30 (d, 1H), 8.19-8.13 (m, 2H), 7.99-7.89 (m, 4H), |
| | 7.77 (d, 1H), 7.58-7.50 (m, 2H), 7.35 (t, 1H), 7.20-7.16 (m, 2H) |
| 2-55 | δ = 8.55 (m, 1H), 8.30 (d, 1H), 8.19-8.13 (m, 2H), 7.99-7.89 (m, 4H), |
| | 7.77 (d, 1H), 7.58-7.50 (m, 2H), 7.35 (t, 1H), 7.20-7.16 (m, 2H) |
| 2-57 | δ = 8.55 (m, 1H), 8.30 (d, 1H), 8.19-8.13 (m, 2H), 7.99-7.89 (m, 4H), |
| | 7.77 (d, 1H), 7.58-7.50 (m, 2H), 7.35 (t, 1H), 7.20-7.16 (m, 2H) |
| 2-60 | δ = 8.55 (m, 1H), 8.30 (d, 1H), 8.19-8.13 (m, 2H), 7.99-7.89 (m, 4H), |
| | 7.77 (d, 1H), 7.58-7.50 (m, 2H), 7.35 (t, 1H), 7.20-7.16 (m, 2H) |
| 2-61 | δ = 7.62-7.50 (m, 10H) |
| 2-62 | δ = 7.79 (m, 4H), 7.68 (m. 4H), 7.52-7.41 (m, 10H) |
| 2-63 | δ = 8.21 (s, 1H) 7.75-7.41 (m, 13H) |
| 2-64 | δ = 9.05 (s, 1H), 8.33-8.25 (m, 4H), 7.94 (d, 1H), 7.70-7.50 (m, 10H) |
| 2-65 | δ = 7.79 (m, 2H), 7.70-7.68 (m, 3H), 7.58-7.41 (m, 13H) |
| 2-66 | δ = 7.92-7.91 (m, 4H), 7.75 (d, 2H), 7.62-7.41 (m, 8H), 7.25 (s, 4H) |
| 2-68 | δ = 8.21 (s, 2H), 7.75-7.60 (m, 8H), 7.49-7.41 (8H) |
| 2-69 | δ = 8.21 (s, 1H), 7.92-7.91 (m, 4H), 7.75-7.60 (m, 6H), 7.49-7.41 (m, |
| | 7H) |
| 2-70 | δ = 8.21 (s, 1H), 7.94-7.91 (m, 5H), 7.75-7.61 (m, 9H), 7.49-7.41 (m, |
| | 7H) |
| 2-74 | δ = 7.94-7.91 (m, 9H), 7.75-7.73 (m, 5H), 7.61 (d, 2H), 7.49-7.41 (m, |
| | 6H) |
| 2-75 | δ = 7.92-7.91 (m, 8H), 7.75 (d, 4H), 7.49-7.41 (m, 6H), 7.25 (s, 4H) |

TABLE 11

| Compound No. | FD-MS | Compound No. | FD-MS |
|---|---|---|---|
| 2-1 | m/z = 484.59 (C₃₆H₂₄N₂ = 484.19) | 2-2 | m/z = 560.69 (C₄₂H₂₈N₂ = 560.23) |
| 2-3 | m/z = 560.69 (C₄₂H₂₈N₂ = 560.23) | 2-4 | m/z = 560.69 (C₄₂H₂₈N₂ = 560.23) |
| 2-5 | m/z = 636.78 (C₄₈H₃₂N₂ = 636.26) | 2-6 | m/z = 636.78 (C₄₈H₃₂N₂ = 636.26) |
| 2-7 | m/z = 636.78 (C₄₈H₃₂N₂ = 636.26) | 2-8 | m/z = 543.65 (C₄₀H₂₆N₂ = 543.21) |
| 2-9 | m/z = 543.65 (C₄₀H₂₆N₂ = 543.21) | 2-10 | m/z = 600.75 (C₄₅H₃₅N₂ = 600.26) |
| 2-11 | m/z = 600.75 (C₄₅H₃₅N₂ = 600.26) | 2-12 | m/z = 724.89 (C₅₅H₃₆N₂ = 724.29) |
| 2-13 | m/z = 724.89 (C₅₅H₃₆N₂ = 724.29) | 2-14 | m/z = 724.89 (C₅₅H₃₆N₂ = 724.29) |
| 2-15 | m/z = 724.89 (C₅₅H₃₆N₂ = 724.29) | 2-16 | m/z = 634.77 (C₄₈H₃₀N₂ = 634.24) |
| 2-17 | m/z = 509.60 (C₃₇H₂₃N₃ = 509.19) | 2-18 | m/z = 742.98 (C₅₄H₃₈N₂Si = 742.28) |
| 2-19 | m/z = 636.78 (C₄₈H₃₂N₂ = 636.26) | 2-20 | m/z = 636.78 (C₄₈H₃₂N₂ = 636.26) |
| 2-21 | m/z = 636.78 (C₄₈H₃₂N₂ = 636.26) | 2-22 | m/z = 712.88 (C₅₄H₃₆N₂ = 712.29) |
| 2-23 | m/z = 712.88 (C₅₄H₃₆N₂ = 712.29) | 2-24 | m/z = 712.88 (C₅₄H₃₆N₂ = 712.29) |
| 2-25 | m/z = 710.86 (C₅₄H₃₄N₂ = 710.27) | 2-26 | m/z = 712.88 (C₅₄H₃₆N₂ = 712.29) |
| 2-27 | m/z = 712.88 (C₅₄H₃₆N₂ = 712.29) | 2-28 | m/z = 712.88 (C₅₄H₃₆N₂ = 712.29) |
| 2-29 | m/z = 712.88 (C₅₄H₃₆N₂ = 712.29) | 2-30 | m/z = 712.88 (C₅₄H₃₆N₂ = 712.29) |
| 2-31 | m/z = 710.86 (C₅₄H₃₄N₂ = 710.27) | 2-32 | m/z = 636.78 (C₄₈H₃₂N₂ = 636.26) |
| 2-33 | m/z = 712.88 (C₅₄H₃₆N₂ = 712.29) | 2-34 | m/z = 712.88 (C₅₄H₃₆N₂ = 712.29) |
| 2-35 | m/z = 788.97 (C₆₀H₄₀N₂ = 788.32) | 2-36 | m/z = 686.84 (C₅₂H₃₄N₂ = 686.27) |
| 2-37 | m/z = 788.97 (C₆₀H₄₀N₂ = 788.32) | 2-38 | m/z = 788.97 (C₆₀H₄₀N₂ = 788.32) |
| 2-39 | m/z = 686.84 (C₅₂H₃₄N₂ = 686.27) | 2-40 | m/z = 686.84 (C₅₂H₃₄N₂ = 686.27) |
| 2-41 | m/z = 494.65 (C₃₆H₁₄D₁₀N₂ = 494.26) | 2-42 | m/z = 654.89 (C₄₈H₁₄D₁₈N₂ = 654.37) |
| 2-43 | m/z = 574.77 (C₄₁H₁₄D₁₄N₂ = 574.31) | 2-44 | m/z = 650.86 (C₄₈H₁₄D₁₆N₂ = 650.34) |
| 2-45 | m/z = 654.89 (C₄₈H₁₄D₁₈N₂ = 654.37) | 2-46 | m/z = 654.89 (C₄₈H₁₄D₁₈N₂ = 654.37) |
| 2-47 | m/z = 654.89 (C₄₈H₁₄D₁₈N₂ = 654.37) | 2-48 | m/z = 654.89 (C₄₈H₁₄D₁₈N₂ = 654.37) |
| 2-49 | m/z = 654.89 (C₄₈H₁₄D₁₈N₂ = 654.37) | 2-50 | m/z = 734.43 (C₅₄H₁₄D₂₂N₂ = 735.03) |
| 2-51 | m/z = 735.01 (C₅₄H₁₄D₂₂N₂ = 734.43) | 2-52 | m/z = 735.01 (C₅₄H₁₄D₂₂N₂ = 734.43) |
| 2-53 | m/z = 730.98 (C₅₄H₁₄D₂₀N₂ = 730.40) | 2-54 | m/z = 735.01 (C₅₄H₁₄D₂₂N₂ = 734.43) |
| 2-55 | m/z = 735.01 (C₅₄H₁₄D₂₂N₂ = 734.43) | 2-56 | m/z = 815.13 (C₆₀H₁₄D₂₆N₂ = 814.48) |
| 2-57 | m/z = 815.13 (C₆₀H₁₄D₂₆N₂ = 814.48) | 2-58 | m/z = 815.13 (C₆₀H₁₄D₂₆N₂ = 814.48) |
| 2-59 | m/z = 815.13 (C₆₀H₁₄D₂₆N₂ = 814.48) | 2-60 | m/z = 666.86 (C₄₈H₁₄D₁₆N₂O = 666.34) |
| 2-61 | m/z = 498.68 (C₃₆H₁₀D₁₄N₂ = 498.28) | 2-62 | m/z = 650.87 (C₄₈H₁₈D₁₄N₂ = 650.34) |
| 2-63 | m/z = 574.77 (C₄₁H₁₄D₁₄N₂ = 574.31) | 2-64 | m/z = 648.85 (C₄₈H₁₆D₁₄N₂ = 648.33) |
| 2-65 | m/z = 650.87 (C₄₈H₁₈D₁₄N₂ = 650.34) | 2-66 | m/z = 650.87 (C₄₈H₁₈D₁₄N₂ = 650.34) |
| 2-67 | m/z = 650.87 (C₄₈H₁₈D₁₄N₂ = 650.34) | 2-68 | m/z = 650.87 (C₄₈H₁₈D₁₄N₂ = 650.34) |
| 2-69 | m/z = 650.87 (C₄₈H₁₈D₁₄N₂ = 650.34) | 2-70 | m/z = 726.38 (C₅₄H₂₂D₁₄N₂ = 726.98) |
| 2-71 | m/z = 726.96 (C₅₄H₂₂D₁₄N₂ = 726.38) | 2-72 | m/z = 726.96 (C₅₄H₂₂D₁₄N₂ = 726.38) |
| 2-73 | m/z = 724.95 (C₅₄H₂₀D₁₄N₂ = 724.36) | 2-74 | m/z = 726.96 (C₅₄H₂₂D₁₄N₂ = 726.38) |
| 2-75 | m/z = 726.96 (C₅₄H₂₂D₁₄N₂ = 726.38) | 2-76 | m/z = 803.06 (C₆₀H₂₆D₁₄N₂ = 802.41) |
| 2-77 | m/z = 803.06 (C₆₀H₂₆D₁₄N₂ = 802.41) | 2-78 | m/z = 803.06 (C₆₀H₂₆D₁₄N₂ = 802.41) |
| 2-79 | m/z = 803.06 (C₆₀H₂₆D₁₄N₂ = 802.41) | 2-80 | m/z = 803.06 (C₆₀H₂₆D₁₄N₂ = 802.41) |
| 2-81 | m/z = 508.74 (C₃₆D₂₄N₂ = 508.34) | 2-82 | m/z = 668.98 (C₄₈D₃₂N₂ = 668.46) |
| 2-83 | m/z = 588.86 (C₄₂D₂₈N₂ = 588.40) | 2-84 | m/z = 664.95 (C₄₈D₃₀N₂ = 664.43) |
| 2-85 | m/z = 668.98 (C₄₈D₃₂N₂ = 668.46) | 2-86 | m/z = 668.98 (C₄₈D₃₂N₂ = 668.46) |
| 2-87 | m/z = 668.98 (C₄₈D₃₂N₂ = 668.46) | 2-88 | m/z = 668.98 (C₄₈D₃₂N₂ = 668.46) |
| 2-89 | m/z = 668.98 (C₄₈D₃₂N₂ = 668.46) | 2-90 | m/z = 748.518 (C₅₄D₃₆N₂ = 749.12) |
| 2-91 | m/z = 749.10 (C₅₄D₃₆N₂ = 748.51) | 2-92 | m/z = 749.10 (C₅₄D₃₆N₂ = 748.51) |
| 2-93 | m/z = 745.07 (C₅₄D₃₄N₂ = 744.49) | 2-94 | m/z = 749.10 (C₅₄D₃₆N₂ = 748.51) |
| 2-95 | m/z = 749.10 (C₅₄D₃₆N₂ = 748.51) | 2-96 | m/z = 829.22 (C₆₀D₄₀N₂ = 828.57) |
| 2-97 | m/z = 829.22 (C₆₀D₄₀N₂ = 828.57) | 2-98 | m/z = 829.22 (C₆₀D₄₀N₂ = 828.57) |
| 2-99 | m/z = 829.22 (C₆₀D₄₀N₂ = 828.57) | 2-100 | m/z = 680.95 (C₄₈D₃₀N₂O = 680.42) |
| 2-101 | m/z = 697.02 (C₄₈D₃₀N₂S = 696.40) | 2-102 | m/z = 829.22 (C₆₀D₄₀N₂ = 828.57) |
| 2-103 | m/z = 632.95 (C₄₅D₃₂N₂ = 632.46) | 2-104 | m/z = 713.07 (C₅₁D₃₆N₂ = 712.51) |

### Experimental Example 1

### Experimental Example 1-1. Manufacture of Organic Light Emitting Device

A glass substrate on which ITO was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and UVO (ultraviolet ozone) treatment was conducted for 5 minutes using UV in a UV cleaner. After that, the substrate was transferred to a plasma cleaner (PT), and after conducting plasma treatment under vacuum for ITO work function and residual film removal, the substrate was transferred to a thermal deposition apparatus for organic deposition.

Subsequently, the chamber was evacuated until the degree of vacuum therein reached 10⁻⁶ torr, and then 4,4′,4″-tris[2-naphthyl(phenyl)amino]triphenylamine (2-TNATA) was evaporated by applying a current to the cell to deposit a hole injection layer on the ITO substrate to a thickness of 600 Å. To another cell in the vacuum deposition apparatus, the following N,N′-bis(α-naphthyl)-N,N′-diphenyl-4,4′-diamine (NPB) was introduced, and evaporated by applying a current to the cell to deposit a hole transport layer on the hole injection layer to a thickness of 300 Å.

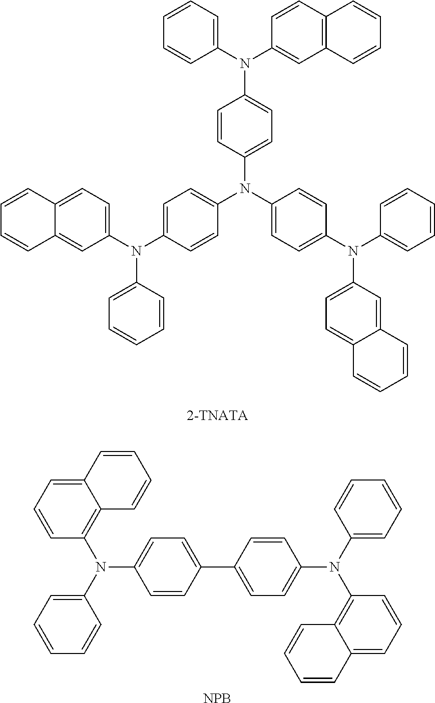 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

A light emitting layer was thermal vacuum deposited thereon as follows. The light emitting layer was deposited to a thickness of 400 Å by depositing the compound described in the following Table 12 as a green host, and, using Ir(ppy)₃ (tris(2-phenylpyridine)iridium) as a green phosphorescent dopant, doping the Ir(ppy)₃ to the host by 7%. After that, BCP was deposited to a thickness of 60 Å as a hole blocking layer, and Alq₃ was deposited to a thickness of 200 Å thereon as an electron transport layer. Lastly, an electron injection layer was formed on the electron transport layer by depositing lithium fluoride (LiF) to a thickness of 10 Å, and then a negative electrode was formed on the electron injection layer by depositing aluminum (Al) to a thickness of 1,200 Å, and as a result, an organic electroluminescent device was manufactured.

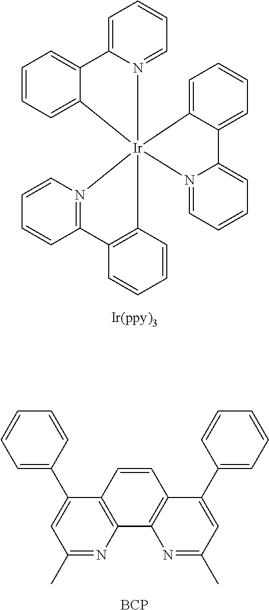

Meanwhile, all the organic compounds required to manufacture the OLED were vacuum sublimation purified under 10⁻⁸ torr to 10⁻⁶ torr by each material to be used in the OLED (organic light emitting device) manufacture.

### Experimental Example 1-2. Driving Voltage and Light Emission Efficiency of Organic Light Emitting Device

For each of the organic light emitting devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc. Results of measuring driving voltage, light emission efficiency, color coordinate (CIE) and lifetime of the organic light emitting devices manufactured according to the present disclosure are shown in the following Table 12.

T₉₀ means a lifetime (unit: hour), a time taken to become 90% with respect to initial luminance.

TABLE 12

| | | | Light | | |
|---|---|---|---|---|---|
| | | Driving | Emission | Color | |
| | | Voltage | Efficiency | Coordinate | Lifetime |
| | Compound | (V) | (cd/A) | (CIE) (x, y) | (T₉₀) |
| Comparative | Ref. 1 | 5.60 | 40.7 | (0.246, 0.717) | 31 |
| Example 1 | | | | | |
| Comparative | Ref. 2 | 5.55 | 42.4 | (0.254, 0.716) | 40 |
| Example 2 | | | | | |
| Comparative | Ref. 3 | 5.15 | 48.1 | (0.254, 0.711) | 45 |
| Example 3 | | | | | |
| Comparative | Ref. 4 | 5.04 | 51.2 | (0.236, 0.699) | 51 |
| Example 4 | | | | | |
| Comparative | Ref. 5 | 5.17 | 47.5 | (0.246, 0.696) | 44 |
| Example 5 | | | | | |
| Comparative | Ref. 6 | 5.06 | 52.3 | (0.251, 0.683) | 52 |
| Example 6 | | | | | |
| Comparative | Ref. 7 | 5.05 | 50.5 | (0.239, 0.725) | 50 |
| Example 7 | | | | | |
| Comparative | Ref. 8 | 5.14 | 51.1 | (0.243, 0.712) | 49 |
| Example 8 | | | | | |
| Comparative | Ref. 9 | 5.42 | 43.3 | (0.230, 0.690) | 33 |
| Example 9 | | | | | |
| Comparative | Ref. 10 | 5.48 | 42.7 | (0.242, 0.719) | 35 |
| Example 10 | | | | | |
| Comparative | Ref. 11 | 5.52 | 40.9 | (0.249, 0.713) | 38 |
| Example 11 | | | | | |
| Comparative | Ref. 12 | 5.35 | 45.5 | (0.239, 0.711) | 31 |
| Example 12 | | | | | |
| Comparative | Ref. 13 | 5.10 | 53.3 | (0.242, 0.712) | 49 |
| Example 13 | | | | | |
| Comparative | Ref. 14 | 5.12 | 51.8 | (0.239, 0.715) | 44 |
| Example 14 | | | | | |
| Comparative | Ref. 15 | 5.42 | 45.1 | (0.245, 0.721) | 37 |
| Example 15 | | | | | |
| Comparative | Ref. 16 | 5.44 | 46.7 | (0.244, 0.719) | 33 |
| Example 16 | | | | | |
| Comparative | Ref. 17 | 5.01 | 35.1 | (0.239, 0.711) | 52 |
| Example 17 | | | | | |
| Comparative | Ref. 18 | 5.13 | 33.7 | (0.233, 0.715) | 56 |
| Example 18 | | | | | |
| Comparative | Ref. 19 | 5.05 | 37.6 | (0.243, 0.722) | 55 |
| Example 19 | | | | | |
| Comparative | Ref. 20 | 5.09 | 30.9 | (0.242, 0.725) | 51 |
| Example 20 | | | | | |
| Comparative | Ref. 21 | 5.03 | 52.3 | (0.238, 0.712) | 75 |
| Example 21 | | | | | |
| Comparative | Ref. 22 | 4.99 | 53.1 | (0.238, 0.711) | 66 |
| Example 22 | | | | | |
| Comparative | Ref. 23 | 5.43 | 39.3 | (0.241, 0.711) | 59 |
| Example 23 | | | | | |
| Comparative | Ref. 24 | 5.32 | 41.7 | (0.241, 0.712) | 48 |
| Example 24 | | | | | |
| Example 1 | 1-1 | 4.77 | 78.0 | (0.238, 0.711) | 170 |
| Example 2 | 1-2 | 4.45 | 72.5 | (0.241, 0.712) | 182 |
| Example 3 | 1-3 | 4.56 | 69.1 | (0.238, 0.711) | 179 |
| Example 4 | 1-4 | 4.57 | 81.2 | (0.239, 0.714) | 165 |
| Example 5 | 1-5 | 4.41 | 57.2 | (0.245, 0.715) | 250 |
| Example 6 | 1-6 | 4.60 | 72.5 | (0.241, 0.713) | 177 |
| Example 7 | 1-11 | 4.23 | 62.1 | (0.243, 0.714) | 205 |
| Example 8 | 1-12 | 4.47 | 71.8 | (0.239, 0.713) | 189 |
| Example 9 | 1-13 | 4.29 | 66.2 | (0.241, 0.712) | 234 |
| Example 10 | 1-14 | 4.58 | 72.9 | (0.241, 0.715) | 197 |
| Example 11 | 1-15 | 4.75 | 84.4 | (0.242, 0.712) | 155 |
| Example 12 | 1-17 | 4.71 | 70.5 | (0.245, 0.711) | 157 |
| Example 13 | 1-21 | 4.53 | 74.5 | (0.241, 0.716) | 203 |
| Example 14 | 1-22 | 4.31 | 60.9 | (0.238, 0.715) | 221 |
| Example 15 | 1-23 | 4.59 | 85.0 | (0.238, 0.713) | 143 |
| Example 16 | 1-24 | 4.65 | 79.9 | (0.241, 0.711) | 149 |
| Example 17 | 1-27 | 4.60 | 80.1 | (0.240, 0.714) | 185 |
| Example 18 | 1-33 | 4.69 | 81.1 | (0.239, 0.714) | 155 |
| Example 19 | 1-34 | 4.57 | 74.7 | (0.245, 0.713) | 183 |
| Example 20 | 1-35 | 4.45 | 69.8 | (0.244, 0.712) | 201 |
| Example 21 | 1-36 | 4.75 | 80.4 | (0.241, 0.711) | 161 |
| Example 22 | 1-37 | 4.38 | 69.1 | (0.241, 0.715) | 240 |
| Example 23 | 1-38 | 4.51 | 71.9 | (0.239, 0.721) | 201 |
| Example 24 | 1-43 | 4.27 | 65.0 | (0.241, 0.711) | 252 |
| Example 25 | 1-44 | 4.60 | 76.3 | (0.238, 0.725) | 199 |
| Example 26 | 1-47 | 4.78 | 83.2 | (0.238, 0.714) | 138 |
| Example 27 | 1-48 | 4.73 | 75.9 | (0.239, 0.712) | 170 |
| Example 28 | 1-51 | 4.52 | 73.5 | (0.240, 0.711) | 202 |
| Example 29 | 1-52 | 4.23 | 59.6 | (0.241, 0.715) | 219 |
| Example 30 | 1-55 | 4.61 | 82.1 | (0.241, 0.716) | 162 |
| Example 31 | 1-56 | 4.59 | 78.8 | (0.240, 0.711) | 166 |
| Example 32 | 1-61 | 4.52 | 70.9 | (0.243, 0.711) | 169 |
| Example 33 | 1-62 | 4.64 | 81.0 | (0.242, 0.714) | 157 |
| Example 34 | 1-65 | 4.75 | 77.9 | (0.245, 0.720) | 153 |
| Example 35 | 1-66 | 4.71 | 70.8 | (0.239, 0.719) | 161 |
| Example 36 | 1-67 | 4.52 | 84.5 | (0.238, 0.714) | 166 |
| Example 37 | 1-68 | 4.58 | 81.9 | (0.241, 0.713) | 159 |
| Example 38 | 1-77 | 4.71 | 71.1 | (0.241, 0.713) | 149 |
| Example 39 | 1-78 | 4.55 | 78.0 | (0.239, 0.714) | 201 |
| Example 40 | 1-79 | 4.53 | 74.7 | (0.238, 0.717) | 198 |
| Example 41 | 1-80 | 4.62 | 80.2 | (0.245, 0.715) | 141 |
| Example 42 | 1-81 | 4.24 | 62.9 | (0.245, 0.717) | 228 |
| Example 43 | 1-82 | 4.48 | 71.8 | (0.239, 0.716) | 174 |
| Example 44 | 1-83 | 4.73 | 83.3 | (0.241, 0.712) | 157 |
| Example 45 | 1-84 | 4.70 | 75.6 | (0.240, 0.712) | 159 |
| Example 46 | 1-87 | 4.29 | 67.7 | (0.243, 0.713) | 217 |
| Example 47 | 1-88 | 4.57 | 69.9 | (0.242, 0.715) | 178 |
| Example 48 | 1-91 | 4.60 | 81.4 | (0.241, 0.711) | 142 |
| Example 49 | 1-92 | 4.73 | 82.1 | (0.239, 0.711) | 148 |
| Example 50 | 1-95 | 4.57 | 74.5 | (0.241, 0.712) | 195 |
| Example 51 | 1-96 | 4.25 | 68.6 | (0.241, 0.717) | 234 |
| Example 52 | 1-99 | 4.50 | 72.6 | (0.242, 0.718) | 155 |
| Example 53 | 1-100 | 4.57 | 76.7 | (0.243, 0.715) | 153 |
| Example 54 | 1-105 | 4.73 | 71.8 | (0.241, 0.711) | 161 |
| Example 55 | 1-106 | 4.75 | 80.6 | (0.248, 0.712) | 158 |
| Example 56 | 1-109 | 4.58 | 77.2 | (0.240, 0.714) | 177 |
| Example 57 | 1-121 | 4.52 | 83.4 | (0.244, 0.713) | 144 |
| Example 58 | 1-122 | 4.60 | 77.4 | (0.238, 0.713) | 171 |
| Example 59 | 1-123 | 4.58 | 72.9 | (0.241, 0.711) | 178 |
| Example 60 | 1-124 | 4.71 | 85.0 | (0.244, 0.714) | 149 |
| Example 61 | 1-131 | 4.27 | 59.9 | (0.241, 0.713) | 235 |
| Example 62 | 1-132 | 4.45 | 65.5 | (0.242, 0.712) | 204 |
| Example 63 | 1-135 | 4.59 | 80.8 | (0.243, 0.711) | 154 |
| Example 64 | 1-136 | 4.51 | 70.9 | (0.241, 0.711) | 159 |
| Example 65 | 1-141 | 4.49 | 72.2 | (0.239, 0.714) | 188 |
| Example 66 | 1-142 | 4.23 | 61.8 | (0.238, 0.714) | 235 |
| Example 67 | 1-143 | 4.51 | 83.4 | (0.241, 0.715) | 161 |
| Example 68 | 1-144 | 4.73 | 81.1 | (0.241, 0.718) | 163 |
| Example 69 | 1-153 | 4.63 | 80.1 | (0.240, 0.714) | 181 |
| Example 70 | 1-193 | 4.83 | 71.5 | (0.243, 0.715) | 164 |
| Example 71 | 1-194 | 4.68 | 70.7 | (0.242, 0.716) | 166 |
| Example 72 | 1-195 | 4.62 | 58.8 | (0.243, 0.715) | 244 |
| Example 73 | 1-196 | 4.71 | 69.5 | (0.242, 0.716) | 196 |
| Example 74 | 1-205 | 4.69 | 72.5 | (0.242, 0.715) | 188 |
| Example 75 | 1-206 | 4.63 | 70.1 | (0.243, 0.713) | 143 |
| Example 76 | 1-207 | 4.57 | 73.2 | (0.242, 0.714) | 199 |
| Example 77 | 1-208 | 4.40 | 59.9 | (0.244, 0.716) | 257 |
| Example 78 | 1-221 | 4.71 | 73.3 | (0.243, 0.715) | 160 |
| Example 79 | 1-222 | 4.77 | 75.1 | (0.242, 0.712) | 145 |
| Example 80 | 1-223 | 4.45 | 68.1 | (0.245, 0.712) | 207 |
| Example 81 | 1-224 | 4.59 | 67.9 | (0.244, 0.715) | 180 |
| Example 82 | 1-229 | 4.75 | 72.5 | (0.236, 0.711) | 183 |
| Example 83 | 1-230 | 4.77 | 77.9 | (0.237, 0.712) | 175 |
| Example 84 | 1-231 | 4.77 | 80.5 | (0.238, 0.712) | 188 |
| Example 85 | 1-241 | 4.40 | 57.2 | (0.245, 0.715) | 256 |
| Example 86 | 1-242 | 4.41 | 56.8 | (0.243, 0.715) | 250 |
| Example 87 | 1-243 | 4.40 | 57.5 | (0.245, 0.715) | 259 |
| Example 88 | 1-244 | 4.61 | 72.5 | (0.241, 0.713) | 181 |
| Example 89 | 1-245 | 4.60 | 71.8 | (0.240, 0.713) | 170 |
| Example 90 | 1-246 | 4.62 | 73.1 | (0.242, 0.713) | 183 |
| Example 91 | 1-247 | 4.75 | 84.6 | (0.241, 0.712) | 158 |
| Example 92 | 1-248 | 4.77 | 85.4 | (0.242, 0.712) | 150 |
| Example 93 | 1-249 | 4.74 | 83.1 | (0.243, 0.713) | 153 |
| Example 94 | 1-265 | 4.29 | 67.2 | (0.242, 0.712) | 245 |
| Example 95 | 1-266 | 4.30 | 66.8 | (0.241, 0.712) | 233 |
| Example 96 | 1-267 | 4.29 | 66.5 | (0.241, 0.712) | 241 |
| Example 97 | 1-268 | 4.57 | 69.5 | (0.240, 0.715) | 188 |
| Example 98 | 1-269 | 4.57 | 70.9 | (0.241, 0.715) | 199 |
| Example 99 | 1-270 | 4.58 | 71.1 | (0.240, 0.714) | 197 |
| Example 100 | 1-316 | 4.62 | 59.9 | (0.242, 0.713) | 249 |
| Example 101 | 1-317 | 4.62 | 60.5 | (0.243, 0.713) | 257 |
| Example 102 | 1-318 | 4.63 | 61.8 | (0.242, 0.715) | 262 |
| Example 103 | 1-319 | 4.69 | 71.7 | (0.241, 0.714) | 180 |
| Example 104 | 1-320 | 4.69 | 75.9 | (0.241, 0.715) | 189 |
| Example 105 | 1-321 | 4.68 | 73.8 | (0.241, 0.715) | 190 |
| Example 106 | 1-322 | 4.61 | 69.5 | (0.243, 0.713) | 143 |
| Example 107 | 1-323 | 4.60 | 70.1 | (0.243, 0.713) | 143 |
| Example 108 | 1-324 | 4.61 | 70.1 | (0.243, 0.713) | 143 |
| Example 109 | 1-325 | 4.57 | 70.1 | (0.243, 0.715) | 190 |
| Example 110 | 1-326 | 4.56 | 71.5 | (0.243, 0.715) | 188 |
| Example 111 | 1-327 | 4.56 | 70.6 | (0.244, 0.715) | 195 |
| Example 112 | 1-331 | 4.65 | 82.0 | (0.240, 0.712) | 170 |
| Example 113 | 1-332 | 4.52 | 77.0 | (0.240, 0.713) | 193 |
| Example 114 | 1-335 | 4.23 | 61.5 | (0.241, 0.712) | 235 |

[Comparative Compounds Ref. 1 to Ref. 24]

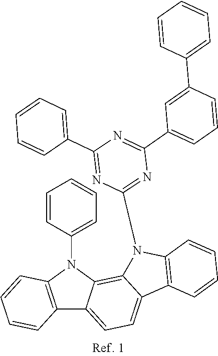 [SMILES: c1ccc(-c2cccc(-c3nc(-c4ccccc4)nc(-n4c5ccccc5c5ccc6c7ccccc7n(-c7ccccc7)c6c54)n3)c2)cc1]

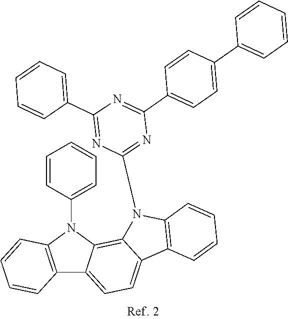 [SMILES: c1ccc(-c2ccc(-c3nc(-c4ccccc4)nc(-n4c5ccccc5c5ccc6c7ccccc7n(-c7ccccc7)c6c54)n3)cc2)cc1]

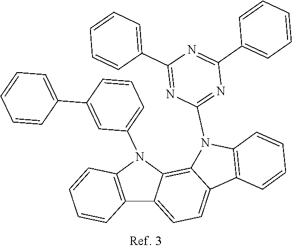 [SMILES: c1ccc(-c2cccc(-n3c4ccccc4c4ccc5c6ccccc6n(-c6nc(-c7ccccc7)nc(-c7ccccc7)n6)c5c43)c2)cc1]

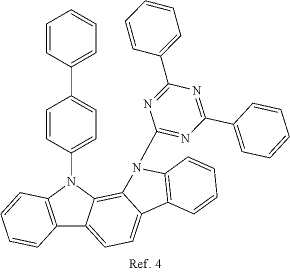 [SMILES: c1ccc(-c2ccc(-n3c4ccccc4c4ccc5c6ccccc6n(-c6nc(-c7ccccc7)nc(-c7ccccc7)n6)c5c43)cc2)cc1]

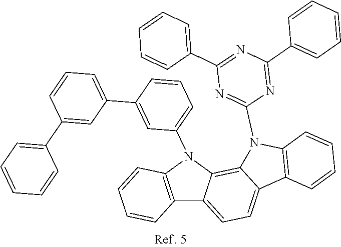 [SMILES: c1ccc(-c2cccc(-c3cccc(-n4c5ccccc5c5ccc6c7ccccc7n(-c7nc(-c8ccccc8)nc(-c8ccccc8)n7)c6c54)c3)c2)cc1]

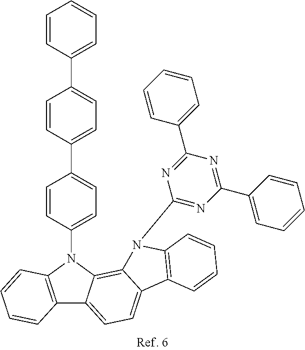 [SMILES: c1ccc(-c2ccc(-c3ccc(-n4c5ccccc5c5ccc6c7ccccc7n(-c7nc(-c8ccccc8)nc(-c8ccccc8)n7)c6c54)cc3)cc2)cc1]

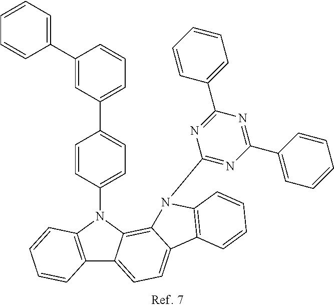 [SMILES: c1ccc(-c2cccc(-c3ccc(-n4c5ccccc5c5ccc6c7ccccc7n(-c7nc(-c8ccccc8)nc(-c8ccccc8)n7)c6c54)cc3)c2)cc1]

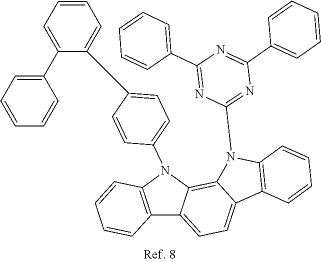 [SMILES: c1ccc(-c2nc(-c3ccccc3)nc(-n3c4ccccc4c4ccc5c6ccccc6n(-c6ccc(-c7ccccc7-c7ccccc7)cc6)c5c43)n2)cc1]

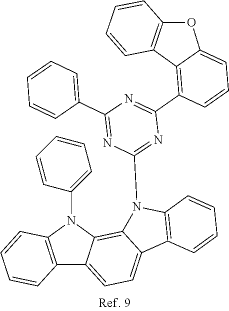 [SMILES: c1ccc(-c2nc(-c3cccc4oc5ccccc5c34)nc(-n3c4ccccc4c4ccc5c6ccccc6n(-c6ccccc6)c5c43)n2)cc1]

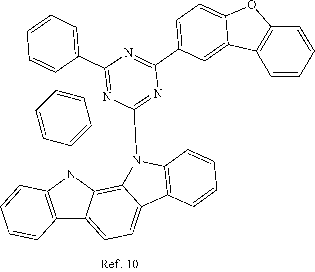 [SMILES: c1ccc(-c2nc(-c3ccc4oc5ccccc5c4c3)nc(-n3c4ccccc4c4ccc5c6ccccc6n(-c6ccccc6)c5c43)n2)cc1]

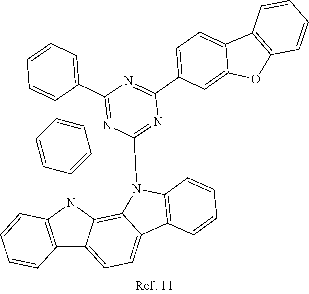 [SMILES: c1ccc(-c2nc(-c3ccc4c(c3)oc3ccccc34)nc(-n3c4ccccc4c4ccc5c6ccccc6n(-c6ccccc6)c5c43)n2)cc1]

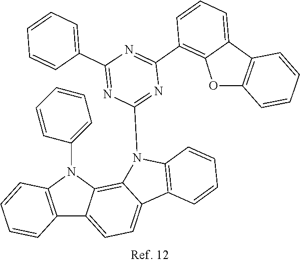 [SMILES: c1ccc(-c2nc(-c3cccc4c3oc3ccccc34)nc(-n3c4ccccc4c4ccc5c6ccccc6n(-c6ccccc6)c5c43)n2)cc1]

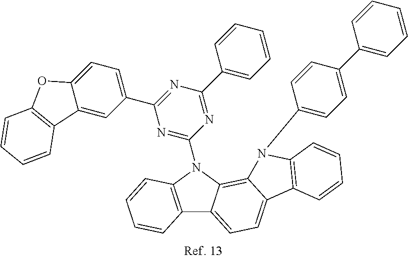 [SMILES: c1ccc(-c2ccc(-n3c4ccccc4c4ccc5c6ccccc6n(-c6nc(-c7ccccc7)nc(-c7ccc8oc9ccccc9c8c7)n6)c5c43)cc2)cc1]

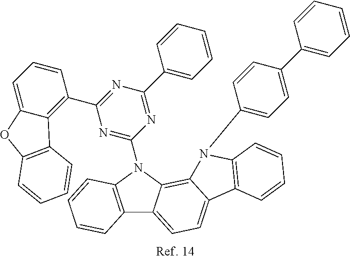 [SMILES: c1ccc(-c2ccc(-n3c4ccccc4c4ccc5c6ccccc6n(-c6nc(-c7ccccc7)nc(-c7cccc8oc9ccccc9c78)n6)c5c43)cc2)cc1]

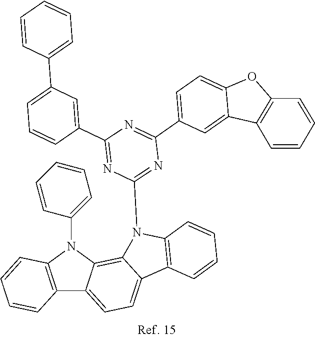 [SMILES: c1ccc(-c2cccc(-c3nc(-c4ccc5oc6ccccc6c5c4)nc(-n4c5ccccc5c5ccc6c7ccccc7n(-c7ccccc7)c6c54)n3)c2)cc1]

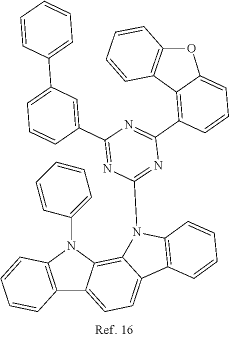 [SMILES: c1ccc(-c2cccc(-c3nc(-c4cccc5oc6ccccc6c45)nc(-n4c5ccccc5c5ccc6c7ccccc7n(-c7ccccc7)c6c54)n3)c2)cc1]

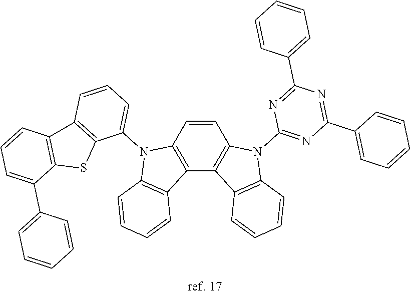 [SMILES: c1ccc(-c2nc(-c3ccccc3)nc(-n3c4ccccc4c4c5c6ccccc6n(-c6cccc7c6sc6c(-c8ccccc8)cccc67)c5ccc43)n2)cc1]

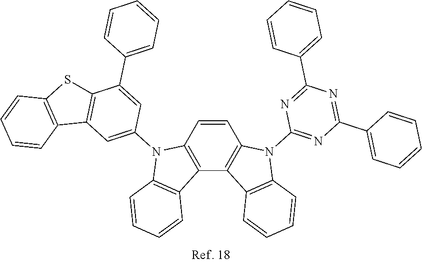 [SMILES: c1ccc(-c2nc(-c3ccccc3)nc(-n3c4ccccc4c4c5c6ccccc6n(-c6cc(-c7ccccc7)c7sc8ccccc8c7c6)c5ccc43)n2)cc1]

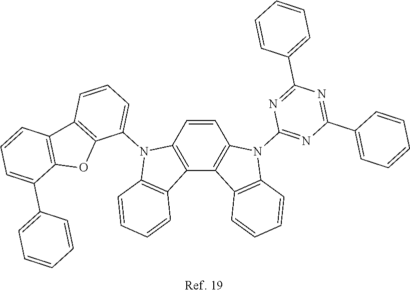 [SMILES: c1ccc(-c2nc(-c3ccccc3)nc(-n3c4ccccc4c4c5c6ccccc6n(-c6cccc7c6oc6c(-c8ccccc8)cccc67)c5ccc43)n2)cc1]

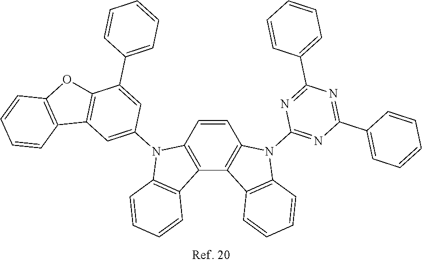 [SMILES: c1ccc(-c2nc(-c3ccccc3)nc(-n3c4ccccc4c4c5c6ccccc6n(-c6cc(-c7ccccc7)c7oc8ccccc8c7c6)c5ccc43)n2)cc1]

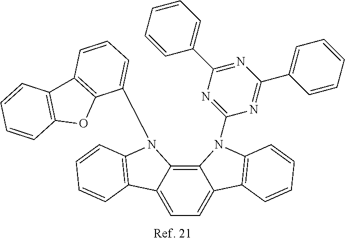 [SMILES: c1ccc(-c2nc(-c3ccccc3)nc(-n3c4ccccc4c4ccc5c6ccccc6n(-c6cccc7c6oc6ccccc67)c5c43)n2)cc1]

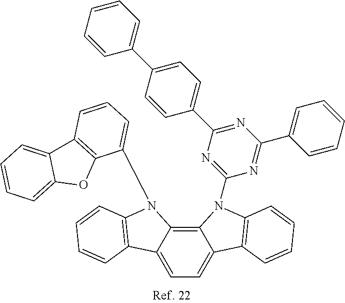 [SMILES: c1ccc(-c2ccc(-c3nc(-c4ccccc4)nc(-n4c5ccccc5c5ccc6c7ccccc7n(-c7cccc8c7oc7ccccc78)c6c54)n3)cc2)cc1]

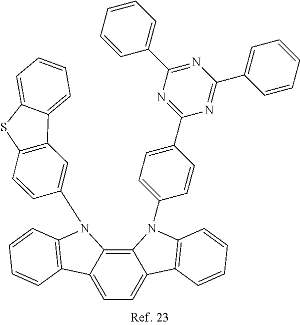 [SMILES: c1ccc(-c2nc(-c3ccccc3)nc(-c3ccc(-n4c5ccccc5c5ccc6c7ccccc7n(-c7ccc8sc9ccccc9c8c7)c6c54)cc3)n2)cc1]

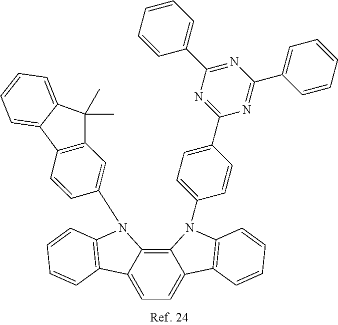 [SMILES: CC1(C)c2ccccc2-c2ccc(-n3c4ccccc4c4ccc5c6ccccc6n(-c6ccc(-c7nc(-c8ccccc8)nc(-c8ccccc8)n7)cc6)c5c43)cc21]

From the results of Table 12, it was identified that the heterocyclic compound of the present disclosure had superior light emission efficiency, and particularly, superior lifetime properties.

Long lifetime properties are the most important factor for commercialization of materials. Through expanding the resonance structure, the heterocyclic compound of the present disclosure is capable of effectively stabilizing electrons by increasing a delocalization rate of the HOMO site. In addition, it is considered that the heterocyclic compound of the present disclosure acts as a sub-donor stabilizing electrons by allowing the triazine to effectively pull electrons from the indolocarbazole, and a lifetime is enhanced thereby.

On the other hand, it was identified that, in Comparative Examples 1 to 16, the HOMO site was localized only to the indolocarbazole and the aryl group failing to effectively stabilize electrons, and a lifetime was reduced thereby.

In addition, in the heterocyclic compound of the present disclosure, a rotatable area between the substituents is reduced due to steric hindrance in the molecule, and light emission efficiency may be increased by forming similar geometries from the ground state to the excited state.

However, it was identified that, in the compounds of Comparative Examples 17 to 24 having weak steric hindrance, various geometries were formed from the ground state to the excited state, and energy was lost due to the generation of various pathways, which reduced light emission efficiency.

In addition, a compound bonding with hydrogen and a compound substituted with deuterium are generally different in thermodynamic behavior. This is due to the fact that the mass of deuterium atom is twice that of hydrogen, and by the difference in the atomic mass, deuterium has lower vibration energy. In addition, a bond length between carbon and deuterium is shorter than a bond with hydrogen, and dissociation energy used to break the bond is also stronger. This is due to the fact that deuterium has a smaller Van der Waals radius compared to hydrogen, resulting in a narrower elongation amplitude of the bond between carbon-deuterium.

Compared to a compound not substituted with deuterium, the compound of the present disclosure substituted with deuterium is capable of having higher light emission efficiency due to the weakening of intermolecular Van der Waals force occurring from the carbon-deuterium having a shorter bond length compared to carbon-hydrogen. In addition, as the carbon-deuterium bond length decreases with the lowering of zero point energy, that is, energy in the ground state, a molecular hardcore volume is reduced, electronical polarizability may be reduced therefrom, and, by weakening intermolecular interactions, a thin film volume may be increased. Such properties create an amorphous state of the thin film and induce an effect of lowering crystallinity. As a result, substitution with deuterium may be effective in enhancing heat resistance of an OLED, which may improve lifetime and driving properties of the device. In addition, the effect of enhancing device properties obtained from the substitution with deuterium is improved as the deuterium substitution ratio increases in the molecule.

### Experimental Example 2

### Experimental Example 2-1. Manufacture of Organic Light Emitting Device

A glass substrate on which ITO was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and UVO (ultraviolet ozone) treatment was conducted for 5 minutes using UV in a UV cleaner. After that, the substrate was transferred to a plasma cleaner (PT), and after conducting plasma treatment under vacuum for ITO work function and residual film removal, the substrate was transferred to a thermal deposition apparatus for organic deposition.

Subsequently, the chamber was evacuated until the degree of vacuum therein reached 10⁻⁶ torr, and then 4,4′,4″-tris[2-naphthyl(phenyl)amino]triphenylamine (2-TNATA) was evaporated by applying a current to the cell to deposit a hole injection layer on the ITO substrate to a thickness of 600 Å. To another cell in the vacuum deposition apparatus, the following N,N′-bis(α-naphthyl)-N,N′-diphenyl-4,4′-diamine (NPB) was introduced, and evaporated by applying a current to the cell to deposit a hole transport layer on the hole injection layer to a thickness of 300 Å.

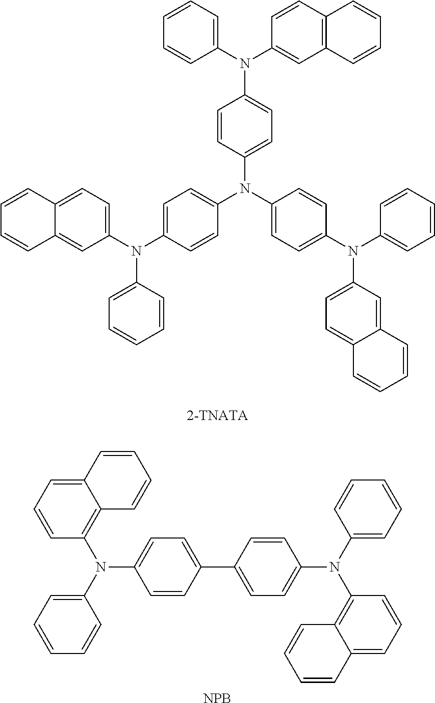 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

A light emitting layer was thermal vacuum deposited thereon as follows. The light emitting layer was deposited to a thickness of 400 Å by pre-mixing and depositing the two types of compounds described in the following Table 13 in one source of supply as a green host, and, using Ir(ppy)₃ as a green phosphorescent dopant, doping the Ir(ppy)₃ to the host by 7% with respect to the deposition thickness of the light emitting layer. After that, BCP was deposited to a thickness of 60 Å as a hole blocking layer, and Alq₃ was deposited to a thickness of 200 Å thereon as an electron transport layer. Lastly, an electron injection layer was formed on the electron transport layer by depositing lithium fluoride (LiF) to a thickness of 10 Å, and then a negative electrode was formed on the electron injection layer by depositing aluminum (Al) to a thickness of 1,200 Å, and as a result, an organic electroluminescent device was manufactured.

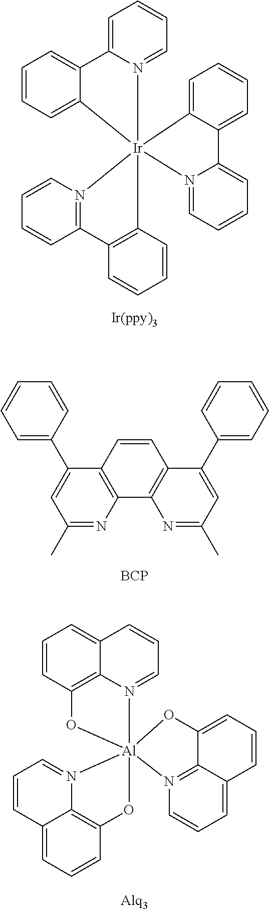

Meanwhile, all the organic compounds required to manufacture the OLED were vacuum sublimation purified under 10⁻⁸ torr to 10⁻⁶ torr by each material to be used in the OLED (organic light emitting device) manufacture.

### Experimental Example 2-2. Driving Voltage and Light Emission Efficiency of Organic Light Emitting Device

For each of the organic light emitting devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc. Results of measuring driving voltage, light emission efficiency, color coordinate (CIE) and lifetime of the organic light emitting devices manufactured according to the present disclosure are shown in the following Table 13.

TABLE 13

| | Compound | Compound | | | Light | | |
|---|---|---|---|---|---|---|---|
| | 1 | 2 | | Driving | Emission | Color | |
| | (N | (P | Ratio | Voltage | Efficiency | Coordinate | Lifetime |
| | Type) | Type) | (N/P) | (V) | (cd/A) | (x, y) | (T₉₀) |
| Comparative | Ref. | 2-3 | 1:1 | 5.10 | 44.7 | (0.245, 0.716) | 48 |
| Example 25 | 1 | | | | | | |
| Comparative | Ref. | 2-3 | 1:2 | 5.16 | 46.5 | (0.245, 0.717) | 51 |
| Example 26 | 1 | | | | | | |
| Comparative | Ref. | 2-3 | 1:3 | 5.21 | 47.1 | (0.246, 0.717) | 55 |
| Example 27 | 1 | | | | | | |
| Comparative | Ref. | 2-32 | 1:1 | 4.61 | 50.4 | (0.253, 0.711) | 77 |
| Example 28 | 3 | | | | | | |
| Comparative | Ref. | 2-32 | 1:2 | 4.69 | 52.9 | (0.254, 0.711) | 81 |
| Example 29 | 3 | | | | | | |
| Comparative | Ref. | 2-32 | 1:3 | 4.73 | 56.1 | (0.254, 0.711) | 88 |
| Example 30 | 3 | | | | | | |
| Comparative | Ref. | 2-88 | 1:1 | 4.49 | 49.4 | (0.251, 0.683) | 64 |
| Example 31 | 6 | | | | | | |
| Comparative | Ref. | 2-88 | 1:2 | 4.58 | 52.5 | (0.251, 0.683) | 73 |
| Example 32 | 6 | | | | | | |
| Comparative | Ref. | 2-88 | 1:3 | 4.63 | 57.1 | (0.251, 0.683) | 77 |
| Example 33 | 6 | | | | | | |
| Comparative | Ref. | 2-85 | 1:1 | 4.97 | 41.5 | (0.249, 0.713) | 48 |
| Example 34 | 11 | | | | | | |
| Comparative | Ref. | 2-85 | 1:2 | 5.06 | 44.8 | (0.249, 0.713) | 55 |
| Example 35 | 11 | | | | | | |
| Comparative | Ref. | 2-85 | 1:3 | 5.11 | 46.1 | (0.249, 0.713) | 59 |
| Example 36 | 11 | | | | | | |
| Comparative | Ref. | 2-32 | 1:1 | 4.57 | 37.2 | (0.239, 0.711) | 85 |
| Example 37 | 17 | | | | | | |
| Comparative | Ref. | 2-32 | 1:2 | 4.62 | 40.6 | (0.239, 0.711) | 91 |
| Example 38 | 17 | | | | | | |
| Comparative | Ref. | 2-32 | 1:3 | 4.67 | 41.7 | (0.239, 0.711) | 98 |
| Example 39 | 17 | | | | | | |
| Comparative | Ref. | 2-100 | 1:1 | 4.55 | 39.8 | (0.243, 0.722) | 81 |
| Example 40 | 19 | | | | | | |
| Comparative | Ref. | 2-100 | 1:2 | 4.61 | 40.7 | (0.243, 0.722) | 85 |
| Example 41 | 19 | | | | | | |
| Comparative | Ref. | 2-100 | 1:3 | 4.68 | 42.3 | (0.243, 0.722) | 88 |
| Example 42 | 19 | | | | | | |
| Example 115 | 1-1 | 2-3 | 1:1 | 4.17 | 82.9 | (0.238, 0.711) | 293 |
| Example 116 | 1-1 | 2-3 | 1:2 | 4.30 | 88.3 | (0.238, 0.711) | 309 |
| Example 117 | 1-1 | 2-3 | 1:3 | 4.35 | 92.6 | (0.238, 0.711) | 317 |
| Example 118 | 1-1 | 2-83 | 1:1 | 4.09 | 84.2 | (0.238, 0.711) | 365 |
| Example 119 | 1-1 | 2-83 | 1:2 | 4.19 | 89.4 | (0.238, 0.711) | 383 |
| Example 120 | 1-1 | 2-83 | 1:3 | 4.25 | 93.8 | (0.238, 0.711) | 420 |
| Example 121 | 1-3 | 2-89 | 1:1 | 3.91 | 80.1 | (0.235, 0.711) | 375 |
| Example 122 | 1-3 | 2-89 | 1:2 | 4.05 | 84.8 | (0.238, 0.711) | 401 |
| Example 123 | 1-3 | 2-89 | 1:3 | 4.20 | 88.3 | (0.238, 0.711) | 432 |
| Example 124 | 1-11 | 2-32 | 1:1 | 3.80 | 71.6 | (0.242, 0.714) | 350 |
| Example 125 | 1-11 | 2-32 | 1:2 | 3.84 | 74.7 | (0.242, 0.714) | 362 |
| Example 126 | 1-11 | 2-32 | 1:3 | 3.92 | 77.8 | (0.242, 0.714) | 369 |
| Example 127 | 1-11 | 2-100 | 1:1 | 3.67 | 73.6 | (0.243, 0.714) | 444 |
| Example 128 | 1-11 | 2-100 | 1:2 | 3.73 | 76.0 | (0.243, 0.714) | 462 |
| Example 129 | 1-11 | 2-100 | 1:3 | 3.80 | 80.6 | (0.243, 0.714) | 481 |
| Example 130 | 1-12 | 2-100 | 1:1 | 3.95 | 82.3 | (0.239, 0.713) | 370 |
| Example 131 | 1-12 | 2-100 | 1:2 | 4.01 | 86.9 | (0.239, 0.713) | 399 |
| Example 132 | 1-12 | 2-100 | 1:3 | 4.15 | 89.2 | (0.239, 0.713) | 406 |
| Example 133 | 1-13 | 2-32 | 1:1 | 3.79 | 76.6 | (0.240, 0.712) | 404 |
| Example 134 | 1-13 | 2-32 | 1:2 | 3.85 | 80.4 | (0.240, 0.712) | 416 |
| Example 135 | 1-13 | 2-32 | 1:3 | 3.97 | 86.8 | (0.241, 0.712) | 425 |
| Example 136 | 1-13 | 2-85 | 1:1 | 3.66 | 78.3 | (0.240, 0.712) | 467 |
| Example 137 | 1-13 | 2-85 | 1:2 | 3.73 | 83.2 | (0.240, 0.712) | 507 |
| Example 138 | 1-13 | 2-85 | 1:3 | 3.89 | 88.9 | (0.241, 0.712) | 526 |
| Example 139 | 1-23 | 2-82 | 1:1 | 4.07 | 92.3 | (0.238, 0.714) | 282 |
| Example 140 | 1-23 | 2-82 | 1:2 | 4.16 | 97.6 | (0.239, 0.714) | 313 |
| Example 141 | 1-23 | 2-82 | 1:3 | 4.33 | 101.8 | (0.239, 0.714) | 342 |
| Example 142 | 1-27 | 2-3 | 1:1 | 4.10 | 92.4 | (0.240, 0.714) | 226 |
| Example 143 | 1-27 | 2-3 | 1:2 | 4.20 | 95.6 | (0.240, 0.714) | 232 |
| Example 144 | 1-27 | 2-3 | 1:3 | 4.26 | 98.7 | (0.240, 0.714) | 246 |
| Example 145 | 1-27 | 2-83 | 1:1 | 3.98 | 93.5 | (0.240, 0.714) | 307 |
| Example 146 | 1-27 | 2-83 | 1:2 | 4.09 | 97.1 | (0.240, 0.714) | 319 |
| Example 147 | 1-27 | 2-83 | 1:3 | 4.14 | 99.4 | (0.240, 0.714) | 342 |
| Example 148 | 1-33 | 2-83 | 1:1 | 4.17 | 87.9 | (0.240, 0.714) | 306 |
| Example 149 | 1-33 | 2-83 | 1:2 | 4.24 | 93.0 | (0.240, 0.714) | 349 |
| Example 150 | 1-33 | 2-83 | 1:3 | 4.39 | 99.8 | (0.240, 0.714) | 369 |
| Example 151 | 1-35 | 2-89 | 1:1 | 3.89 | 77.7 | (0.244, 0.712) | 480 |
| Example 152 | 1-35 | 2-89 | 1:2 | 3.96 | 82.6 | (0.244, 0.712) | 501 |
| Example 153 | 1-35 | 2-89 | 1:3 | 4.11 | 87.9 | (0.244, 0.712) | 527 |
| Example 154 | 1-43 | 2-100 | 1:1 | 3.68 | 69.3 | (0.241, 0.711) | 526 |
| Example 155 | 1-43 | 2-100 | 1:2 | 3.79 | 75.1 | (0.241, 0.711) | 555 |
| Example 156 | 1-43 | 2-100 | 1:3 | 3.92 | 82.3 | (0.241, 0.711) | 565 |
| Example 157 | 1-44 | 2-100 | 1:1 | 4.12 | 85.1 | (0.239, 0.721) | 375 |
| Example 158 | 1-44 | 2-100 | 1:2 | 4.19 | 89.7 | (0.239, 0.721) | 391 |
| Example 159 | 1-44 | 2-100 | 1:3 | 4.35 | 92.5 | (0.239, 0.721) | 433 |
| Example 160 | 1-65 | 2-83 | 1:1 | 3.90 | 85.7 | (0.241, 0.720) | 309 |
| Example 161 | 1-65 | 2-83 | 1:2 | 4.04 | 91.4 | (0.241, 0.720) | 325 |
| Example 162 | 1-65 | 2-83 | 1:3 | 4.15 | 96.5 | (0.241, 0.720) | 346 |
| Example 163 | 1-79 | 2-89 | 1:1 | 4.03 | 82.9 | (0.238, 0.717) | 397 |
| Example 164 | 1-79 | 2-89 | 1:2 | 4.13 | 87.7 | (0.238, 0.717) | 424 |
| Example 165 | 1-79 | 2-89 | 1:3 | 4.26 | 89.7 | (0.238, 0.717) | 445 |
| Example 166 | 1-109 | 2-3 | 1:1 | 3.94 | 84.8 | (0.241, 0.720) | 249 |
| Example 167 | 1-109 | 2-3 | 1:2 | 4.02 | 89.5 | (0.241, 0.720) | 268 |
| Example 168 | 1-109 | 2-3 | 1:3 | 4.15 | 95.3 | (0.241, 0.720) | 279 |
| Example 169 | 1-109 | 2-83 | 1:1 | 3.82 | 87.0 | (0.241, 0.720) | 319 |
| Example 170 | 1-109 | 2-83 | 1:2 | 3.91 | 92.7 | (0.241, 0.720) | 332 |
| Example 171 | 1-109 | 2-83 | 1:3 | 4.10 | 98.9 | (0.241, 0.720) | 351 |
| Example 172 | 1-141 | 2-82 | 1:1 | 4.01 | 78.7 | (0.239, 0.714) | 364 |
| Example 173 | 1-141 | 2-82 | 1:2 | 4.11 | 82.4 | (0.239, 0.714) | 388 |
| Example 174 | 1-141 | 2-82 | 1:3 | 4.24 | 87.3 | (0.239, 0.714) | 406 |
| Example 175 | 1-142 | 2-82 | 1:1 | 3.76 | 71.8 | (0.238, 0.714) | 486 |
| Example 176 | 1-142 | 2-82 | 1:2 | 3.82 | 77.1 | (0.238, 0.714) | 497 |
| Example 177 | 1-142 | 2-82 | 1:3 | 3.90 | 84.4 | (0.238, 0.714) | 534 |
| Example 178 | 1-153 | 2-83 | 1:1 | 3.99 | 95.7 | (0.239, 0.714) | 318 |
| Example 179 | 1-153 | 2-83 | 1:2 | 4.06 | 99.3 | (0.239, 0.714) | 330 |
| Example 180 | 1-153 | 2-83 | 1:3 | 4.19 | 101.6 | (0.239, 0.714) | 351 |
| Example 181 | 1-193 | 2-82 | 1:1 | 4.35 | 79.3 | (0.243, 0.715) | 313 |
| Example 182 | 1-193 | 2-82 | 1:2 | 4.42 | 85.4 | (0.243, 0.715) | 328 |
| Example 183 | 1-193 | 2-82 | 1:3 | 4.51 | 88.3 | (0.243, 0.715) | 319 |
| Example 184 | 1-195 | 2-82 | 1:1 | 4.16 | 64.7 | (0.244, 0.715) | 451 |
| Example 185 | 1-195 | 2-82 | 1:2 | 4.33 | 66.3 | (0.244, 0.715) | 460 |
| Example 186 | 1-195 | 2-82 | 1:3 | 4.45 | 69.6 | (0.244, 0.715) | 477 |
| Example 187 | 1-208 | 2-88 | 1:1 | 3.95 | 64.6 | (0.244, 0.716) | 474 |
| Example 188 | 1-208 | 2-88 | 1 :2 | 4.11 | 66.4 | (0.244, 0.716) | 501 |
| Example 189 | 1-208 | 2-88 | 1:3 | 4.17 | 70.5 | (0.244, 0.716) | 519 |
| Example 190 | 1-222 | 2-82 | 1:1 | 4.29 | 82.6 | (0.242, 0.712) | 271 |
| Example 191 | 1-222 | 2-82 | 1:2 | 4.40 | 87.9 | (0.242, 0.712) | 298 |
| Example 192 | 1-222 | 2-82 | 1:3 | 4.50 | 89.3 | (0.242, 0.712) | 304 |
| Example 193 | 1-229 | 2-83 | 1:1 | 3.90 | 93.0 | (0.238, 0.711) | 411 |
| Example 194 | 1-229 | 2-83 | 1:2 | 3.92 | 91.6 | (0.238, 0.711) | 421 |
| Example 195 | 1-229 | 2-83 | 1:3 | 3.97 | 100.4 | (0.238, 0.711) | 448 |
| Example 196 | 1-230 | 2-83 | 1:1 | 3.79 | 93.6 | (0.238, 0.711) | 421 |
| Example 197 | 1-230 | 2-83 | 1:2 | 3.92 | 98.3 | (0.238, 0.711) | 435 |
| Example 198 | 1-230 | 2-83 | 1:3 | 3.93 | 101.5 | (0.238, 0.711) | 456 |
| Example 199 | 1-231 | 2-83 | 1:1 | 3.77 | 95.4 | (0.238, 0.711) | 432 |
| Example 200 | 1-231 | 2-83 | 1:2 | 3.91 | 99.1 | (0.238, 0.711) | 445 |
| Example 201 | 1-231 | 2-83 | 1:3 | 3.92 | 103.0 | (0.238, 0.711) | 465 |
| Example 202 | 1-265 | 2-85 | 1:1 | 3.36 | 89.0 | (0.240, 0.712) | 509 |
| Example 203 | 1-265 | 2-85 | 1:2 | 3.41 | 91.7 | (0.240, 0.712) | 535 |
| Example 204 | 1-265 | 2-85 | 1:3 | 3.48 | 96.4 | (0.241, 0.712) | 556 |
| Example 205 | 1-266 | 2-85 | 1:1 | 3.36 | 88.4 | (0.240, 0.712) | 505 |
| Example 206 | 1-266 | 2-85 | 1:2 | 3.40 | 93.9 | (0.240, 0.712) | 541 |
| Example 207 | 1-266 | 2-85 | 1:3 | 3.48 | 95.2 | (0.241, 0.712) | 564 |
| Example 208 | 1-267 | 2-85 | 1:1 | 3.34 | 89.0 | (0.240, 0.712) | 519 |
| Example 209 | 1-267 | 2-85 | 1:2 | 3.38 | 89.9 | (0.240, 0.712) | 561 |
| Example 210 | 1-267 | 2-85 | 1:3 | 3.44 | 95.6 | (0.241, 0.712) | 571 |
| Example 211 | 1-331 | 2-88 | 1:1 | 4.06 | 88.8 | (0.240, 0.712) | 382 |
| Example 212 | 1-331 | 2-88 | 1:2 | 4.20 | 91.7 | (0.240, 0.712) | 385 |
| Example 213 | 1-331 | 2-88 | 1:3 | 4.26 | 93.6 | (0.240, 0.712) | 409 |
| Example 214 | 1-332 | 2-88 | 1:1 | 4.04 | 84.7 | (0.240, 0.713) | 388 |
| Example 215 | 1-332 | 2-88 | 1:2 | 4.08 | 87.3 | (0.240, 0.713) | 406 |
| Example 216 | 1-332 | 2-88 | 1:3 | 4.16 | 94.2 | (0.240, 0.713) | 426 |

[Comparative Compounds]

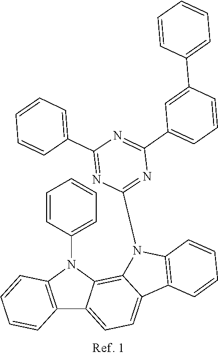 [SMILES: c1ccc(-c2cccc(-c3nc(-c4ccccc4)nc(-n4c5ccccc5c5ccc6c7ccccc7n(-c7ccccc7)c6c54)n3)c2)cc1]

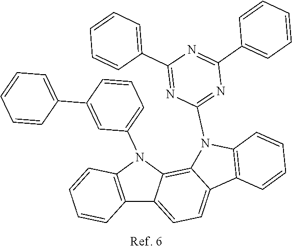 [SMILES: c1ccc(-c2cccc(-n3c4ccccc4c4ccc5c6ccccc6n(-c6nc(-c7ccccc7)nc(-c7ccccc7)n6)c5c43)c2)cc1]

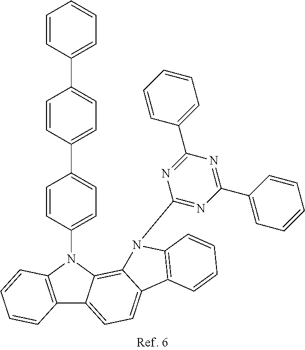 [SMILES: c1ccc(-c2ccc(-c3ccc(-n4c5ccccc5c5ccc6c7ccccc7n(-c7nc(-c8ccccc8)nc(-c8ccccc8)n7)c6c54)cc3)cc2)cc1]

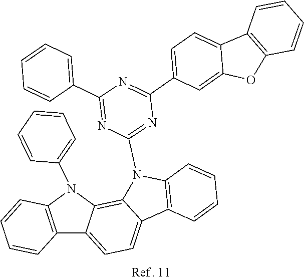 [SMILES: c1ccc(-c2nc(-c3ccc4c(c3)oc3ccccc34)nc(-n3c4ccccc4c4ccc5c6ccccc6n(-c6ccccc6)c5c43)n2)cc1]

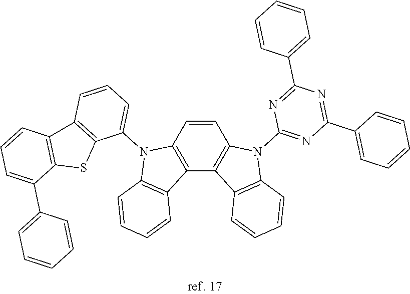 [SMILES: c1ccc(-c2nc(-c3ccccc3)nc(-n3c4ccccc4c4c5c6ccccc6n(-c6cccc7c6sc6c(-c8ccccc8)cccc67)c5ccc43)n2)cc1]

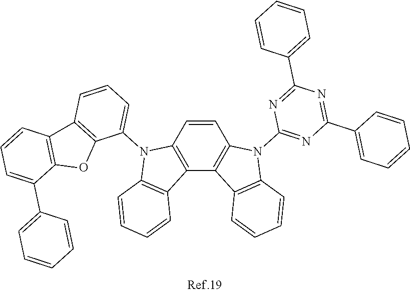 [SMILES: c1ccc(-c2nc(-c3ccccc3)nc(-n3c4ccccc4c4c5c6ccccc6n(-c6cccc7c6oc6c(-c8ccccc8)cccc67)c5ccc43)n2)cc1]

From the results of Table 13, it was seen that effects of more superior efficiency and lifetime were obtained when including Compound 1 (heterocyclic compound represented by Chemical Formula 1) and Compound 2 (heterocyclic compound represented by Chemical Formula 10) at the same time. From such results, it may be predicted that an exciplex phenomenon occurs when including the two compounds at the same time.

The exciplex phenomenon is a phenomenon of releasing energy having sizes of a donor (p-type host) HOMO level and an acceptor (n-type host) LUMO level due to electron exchanges between two molecules. When the exciplex phenomenon occurs between two molecules, reverse intersystem crossing (RISC) occurs, and as a result, internal quantum efficiency of fluorescence may increase up to 100%.

When a donor (p-host) having a favorable hole transport ability and an acceptor (n-host) having a favorable electron transport ability are used as a host of a light emitting layer, holes are injected to the p-host and electrons are injected to the n-host, and a driving voltage may be lowered, which resultantly helps with enhancement in the lifetime.

In the present disclosure, it was identified that more superior device properties were obtained when using the heterocyclic compound represented by Chemical Formula 10 performing a donor role and the heterocyclic compound represented by Chemical Formula 1 performing an acceptor role as the light emitting layer host.

### REFERENCE NUMERAL

100: Substrate
200: Positive Electrode
300: Organic Material Layer
301: Hole Injection Layer
302: Hole Transport Layer
303: Light Emitting Layer
304: Hole Blocking Layer
305: Electron Transport Layer
306: Electron Injection Layer
400: Negative Electrode

1. A heterocyclic compound represented by the following Chemical Formula 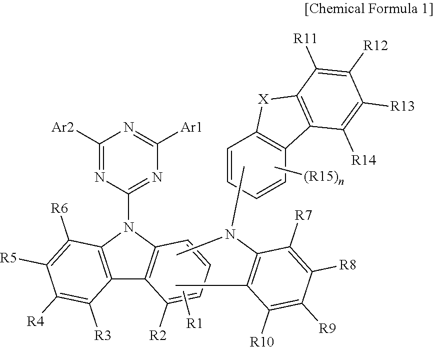
wherein, in Chemical Formula 1,
R1 to R15 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
Ar1 and Ar2 are the same as or different from each other, and each independently a substituted or unsubstituted C6 to C60 aryl group; or a substituted or unsubstituted C2 to C60 heteroaryl group;
X is S; O; CRaRb; or NRc;
n is an integer of 0 to 3, and when n is 2 or greater, R15s are the same as or different from each other;
Ra to Rc are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; and —NR201R202, or two or more groups adjacent to each other bond to each other to form a substituted or unsubstituted C6 to C60 aromatic hydrocarbon ring or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group; and
at least one of R11 to R15 is a substituted or unsubstituted C6 to C60 aryl group; or a substituted or unsubstituted C2 to C60 heteroaryl group.

2. The heterocyclic compound of claim 1, wherein Chemical Formula 1 is represented by any one of the following Chemical Formulae 2 to 5: 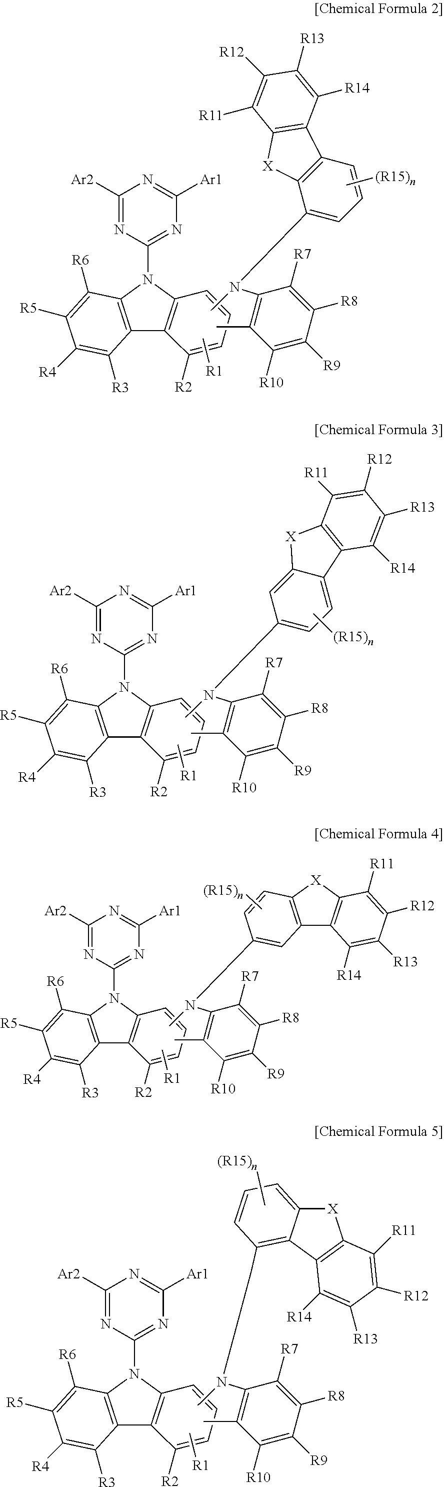
in Chemical Formulae 2 to 5,
R1 to R15, Ar1, Ar2, X and n have the same definitions as in Chemical Formula 1.

3. The heterocyclic compound of claim 1, wherein Chemical Formula 1 is represented by any one of the following Chemical Formulae 6 to 9: 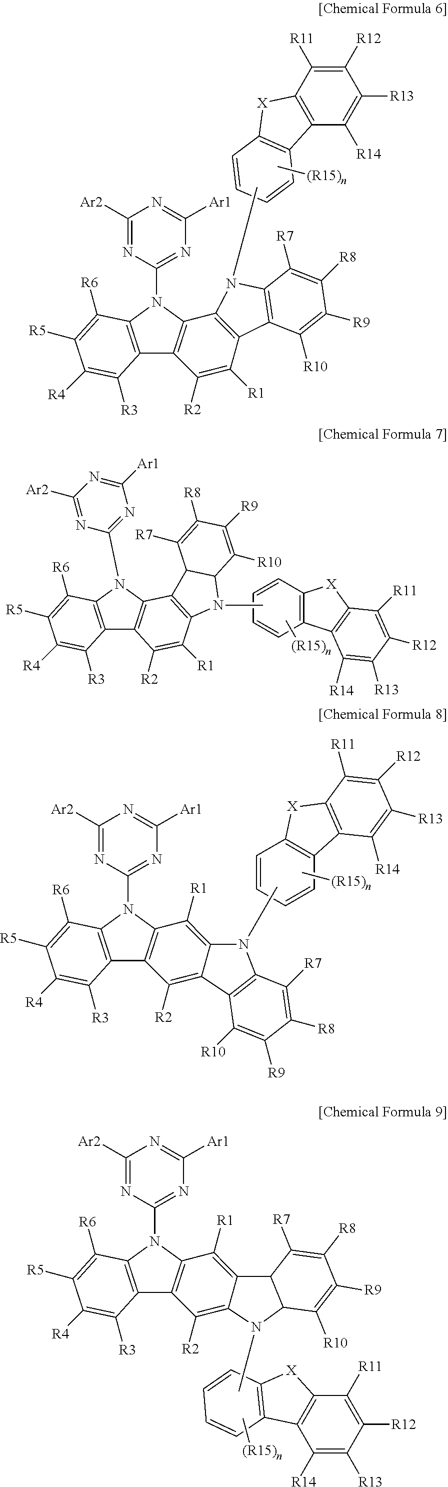
in Chemical Formulae 6 to 9,
R1 to R15, Ar1, Ar2, X and n have the same definitions as in Chemical Formula 1.

4. The heterocyclic compound of claim 1, wherein, when at least one of R11 to R14 is a substituted or unsubstituted C6 to C60 aryl group; or a substituted or unsubstituted C2 to C60 heteroaryl group, R15 is hydrogen; or deuterium; and
when R11 to R14 are the same as or different from each other and each independently hydrogen; or deuterium, n is 1 or greater, and at least one of R15s is a substituted or unsubstituted C6 to C60 aryl group; or a substituted or unsubstituted C2 to C60 heteroaryl group.

5. The heterocyclic compound of claim 1, wherein the heterocyclic compound represented by Chemical Formula 1 does not include deuterium as a substituent, or a content of deuterium is from 1% to 100% based on a total number of hydrogen atoms and deuterium atoms.

6. The heterocyclic compound of claim 1, wherein Chemical Formula 1 is represented by any one of the following compounds:

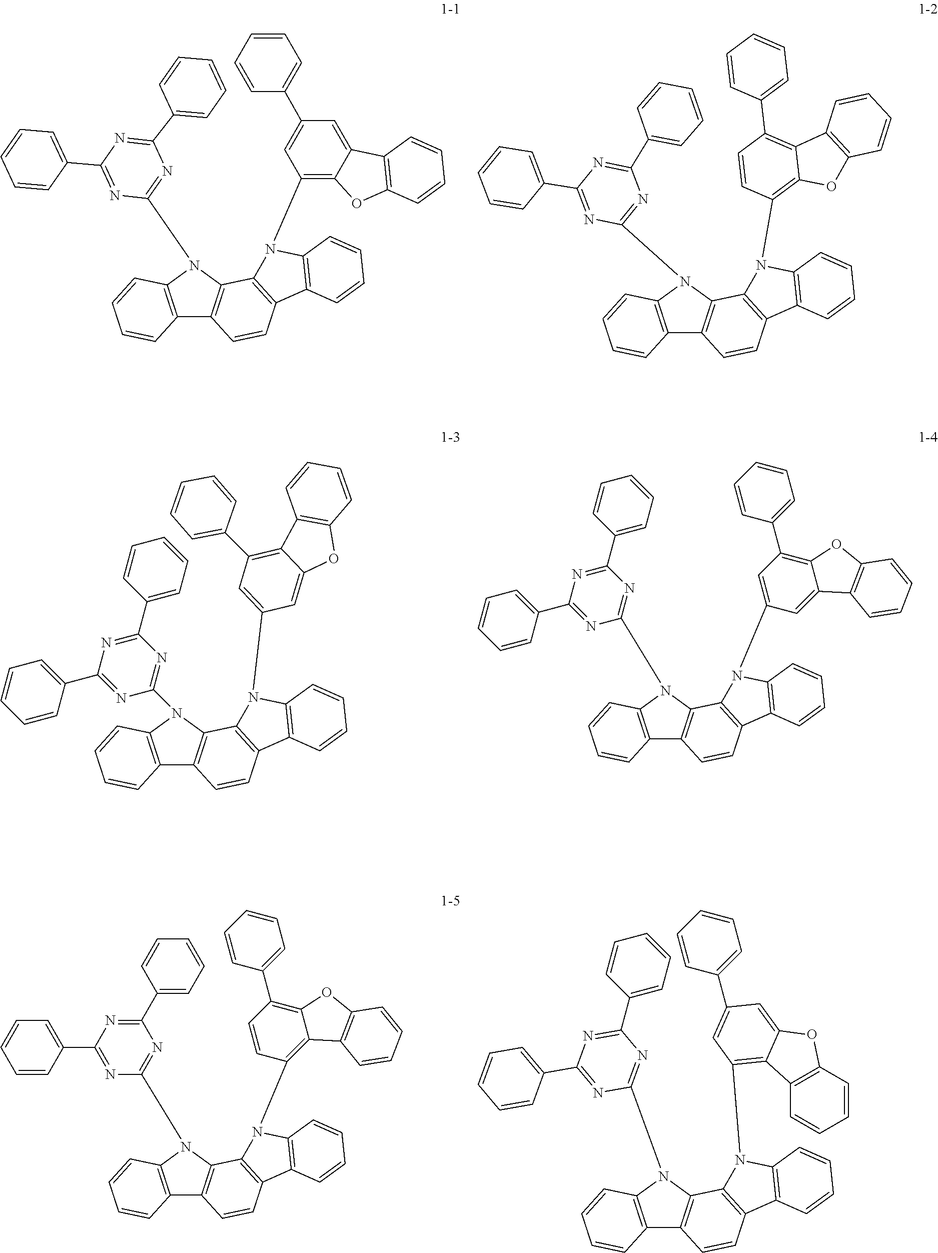

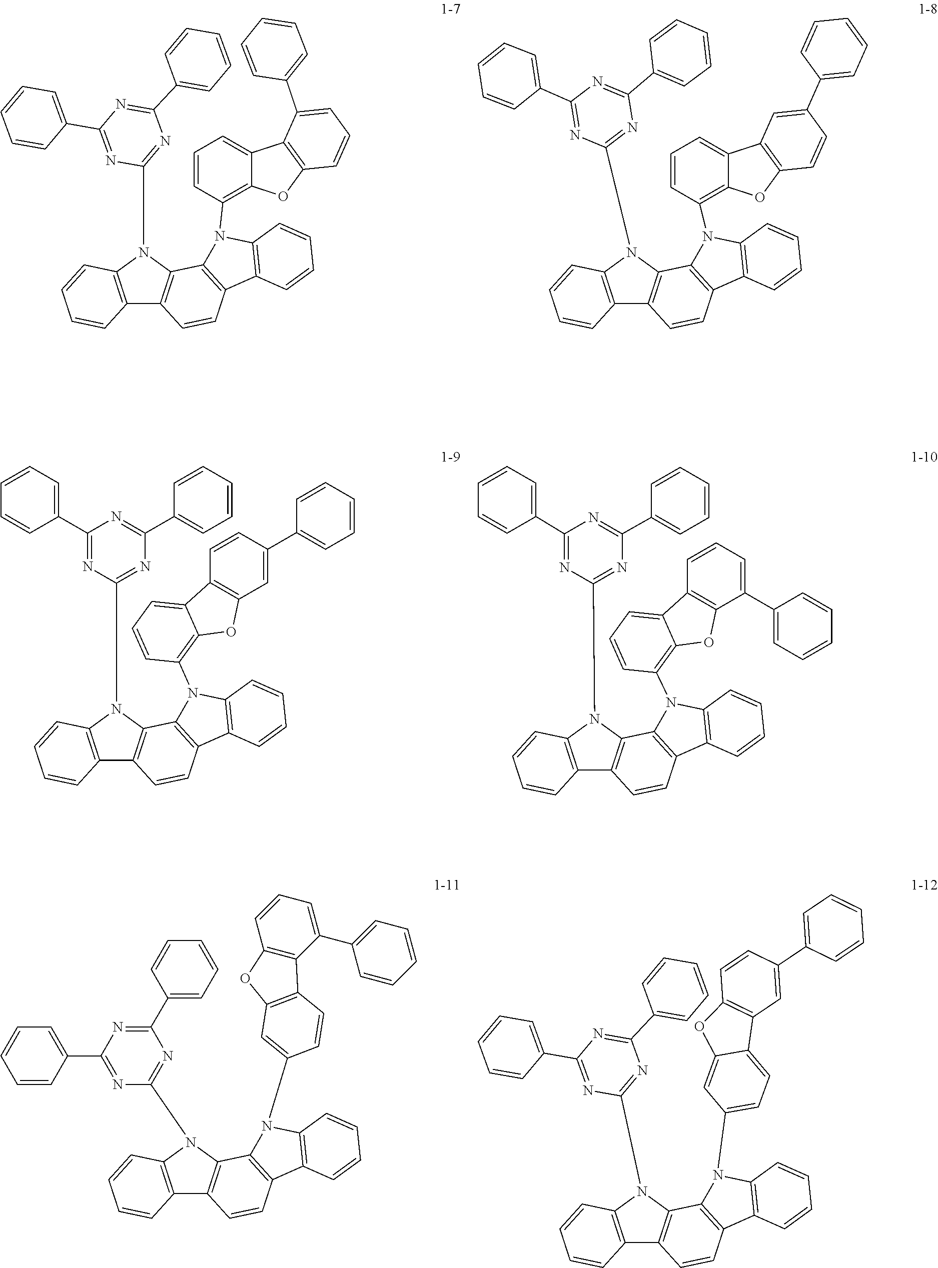

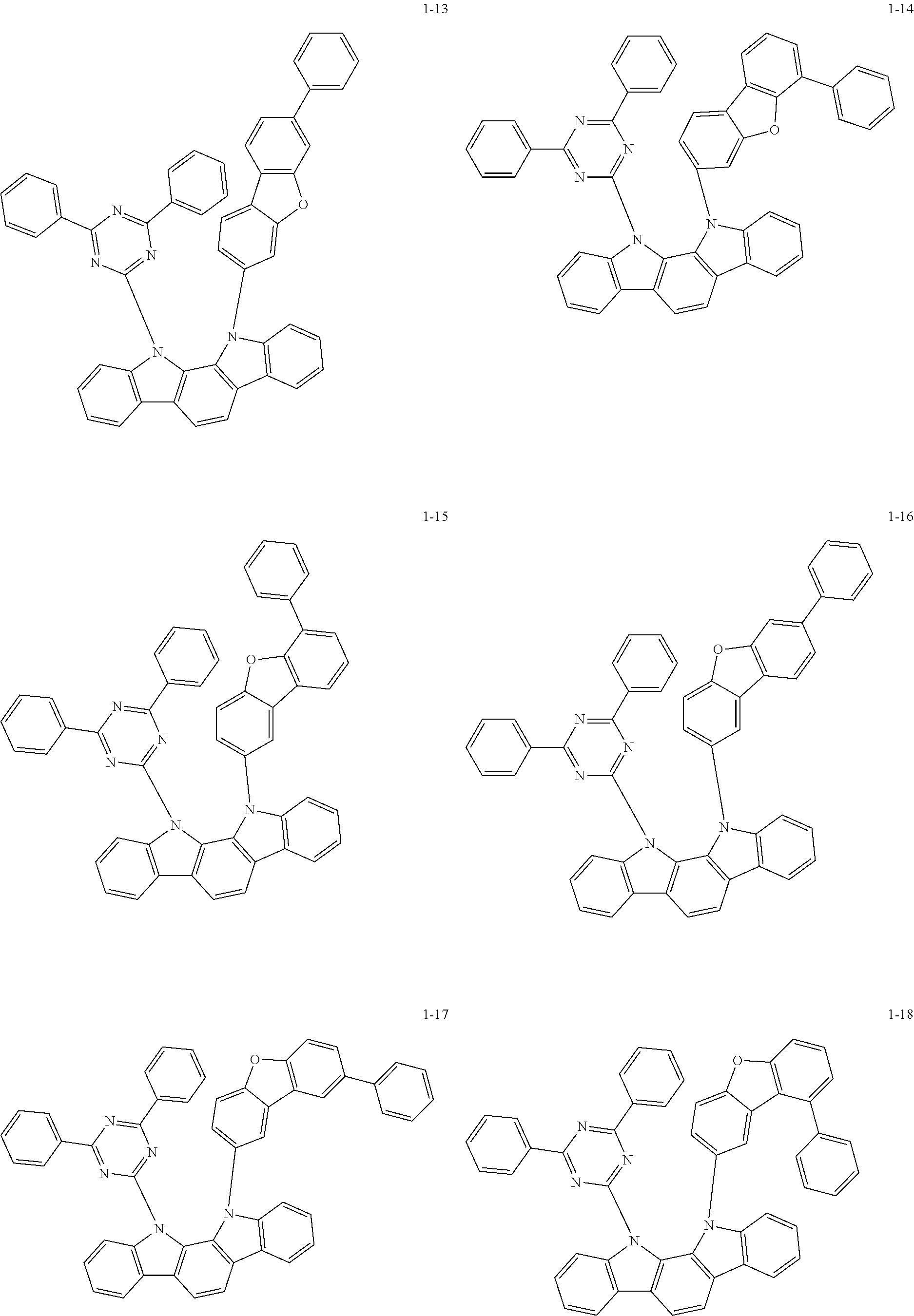

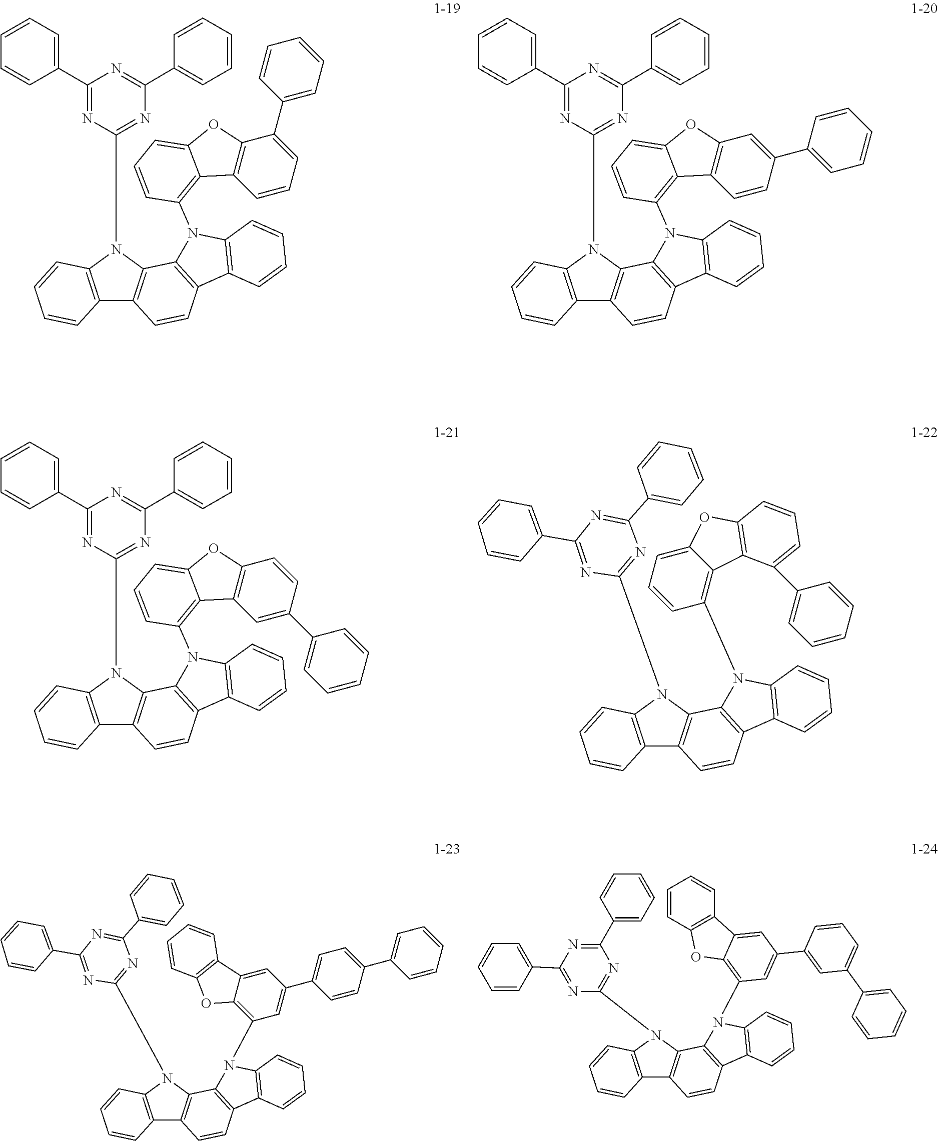

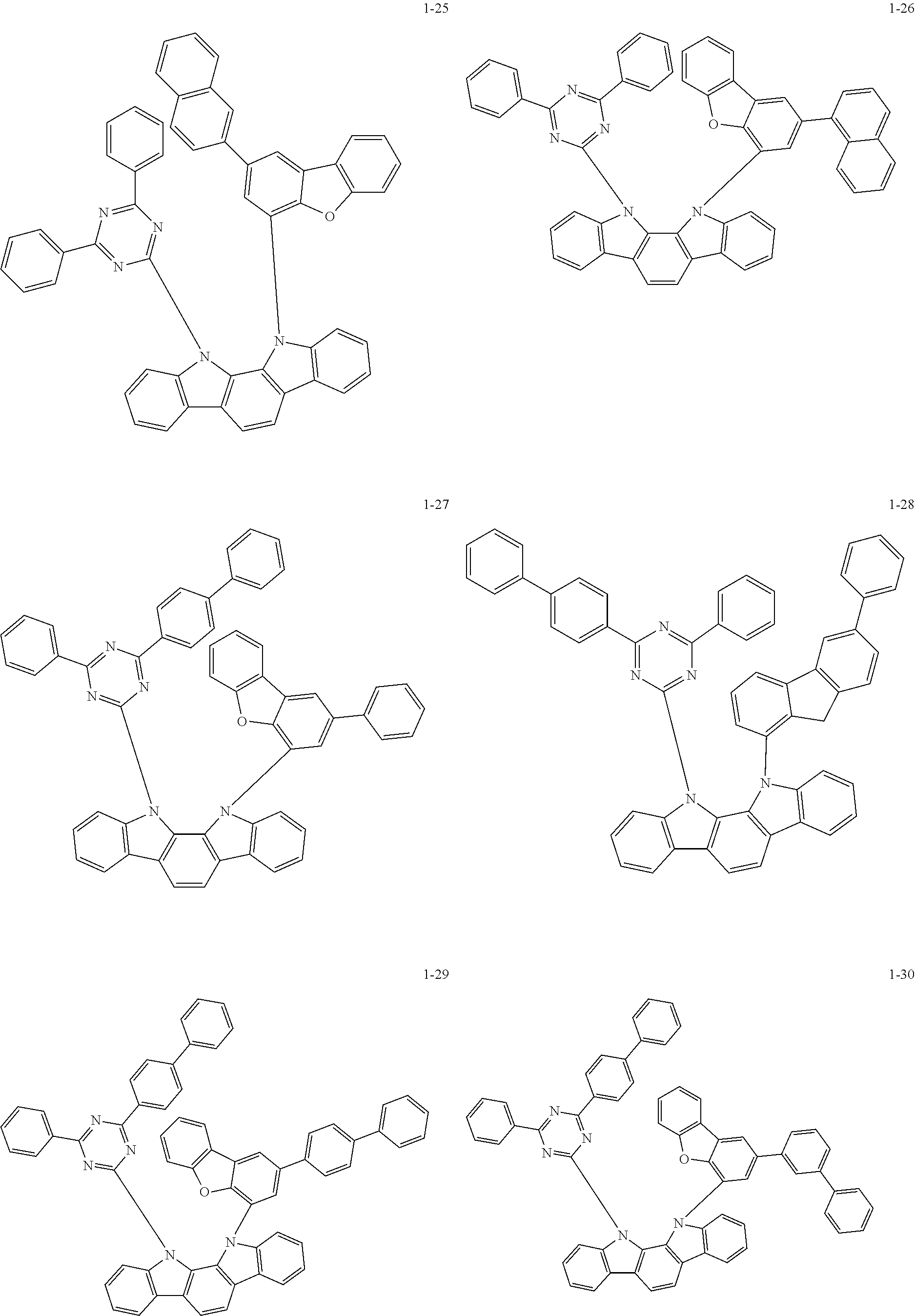

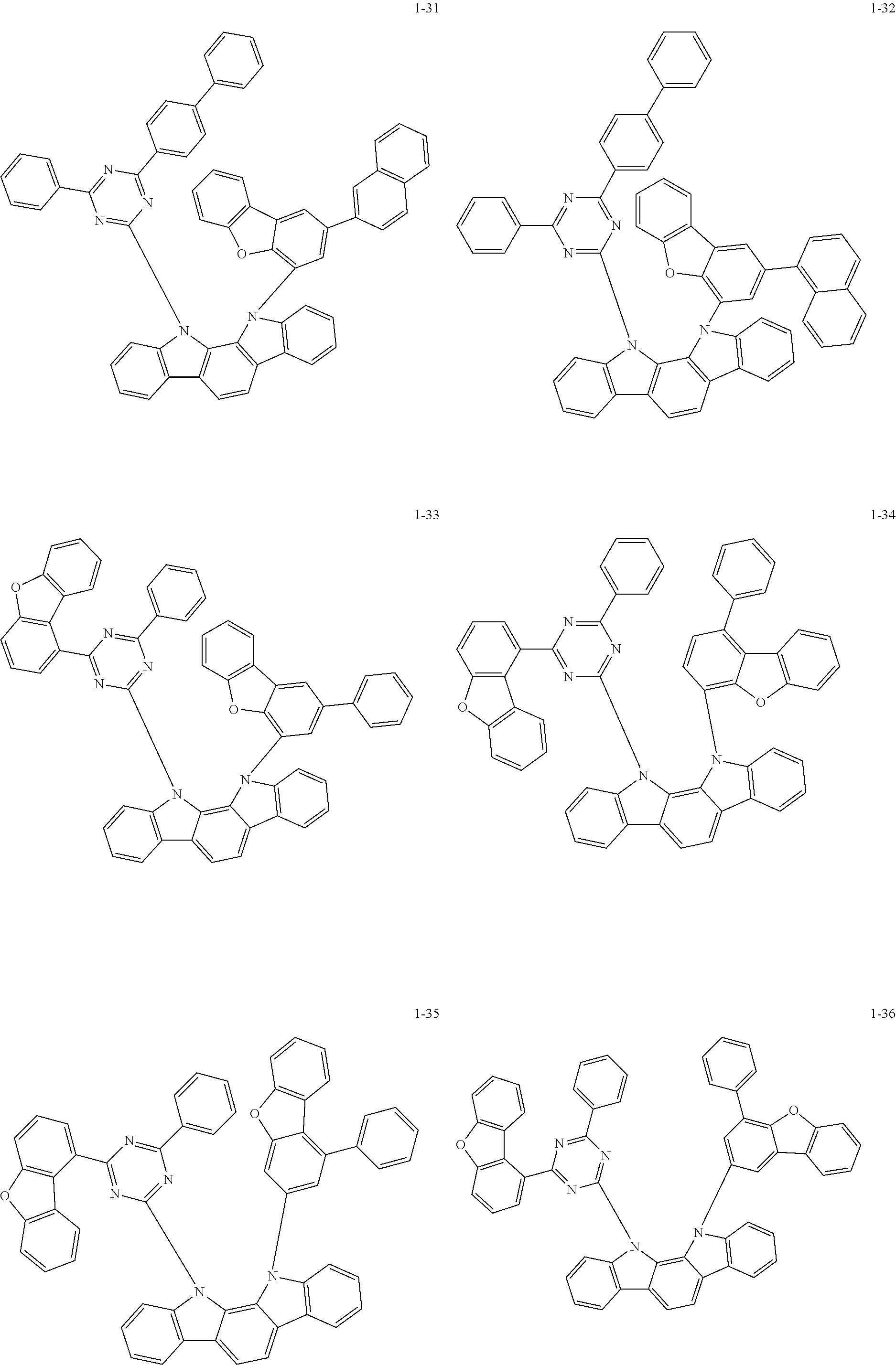

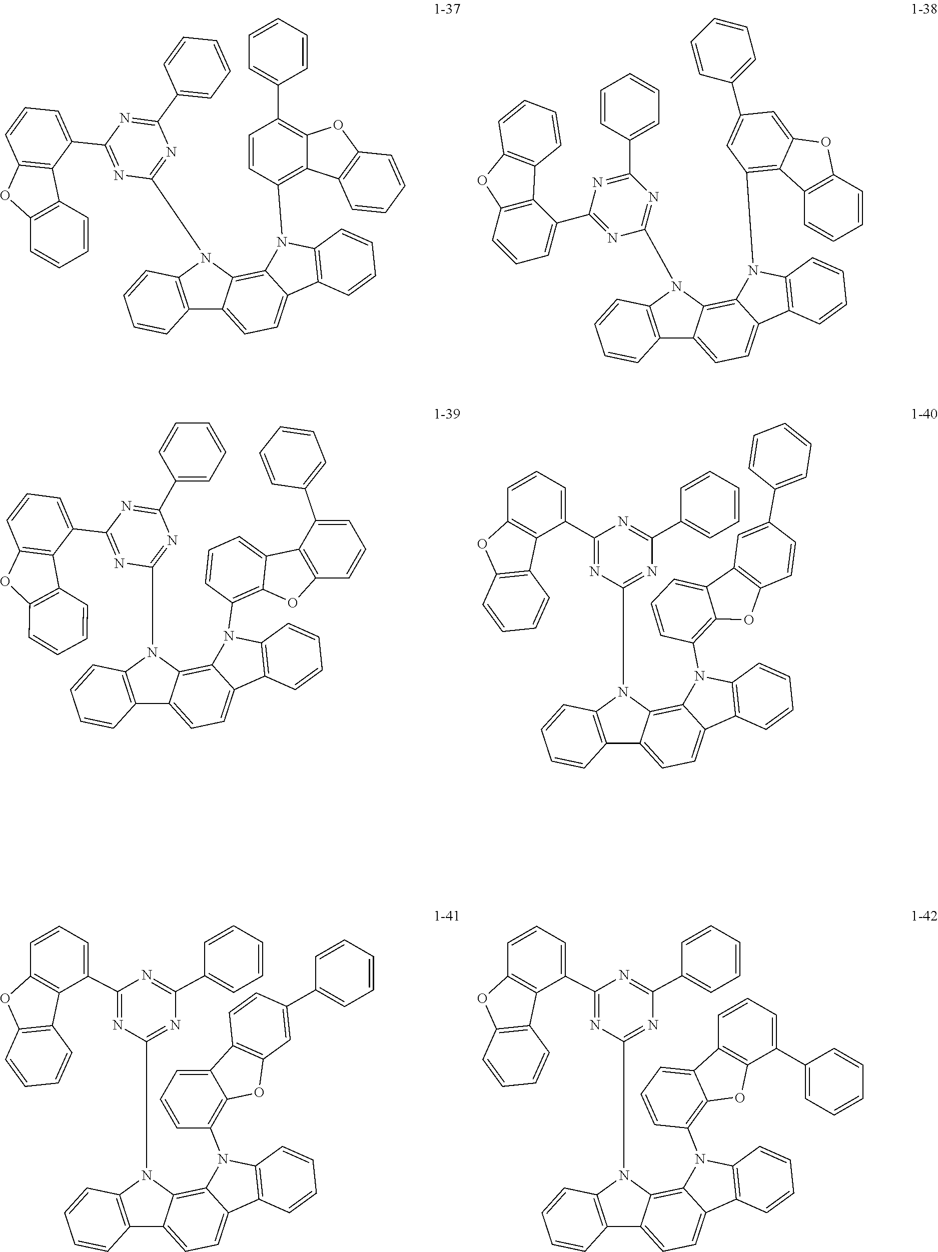

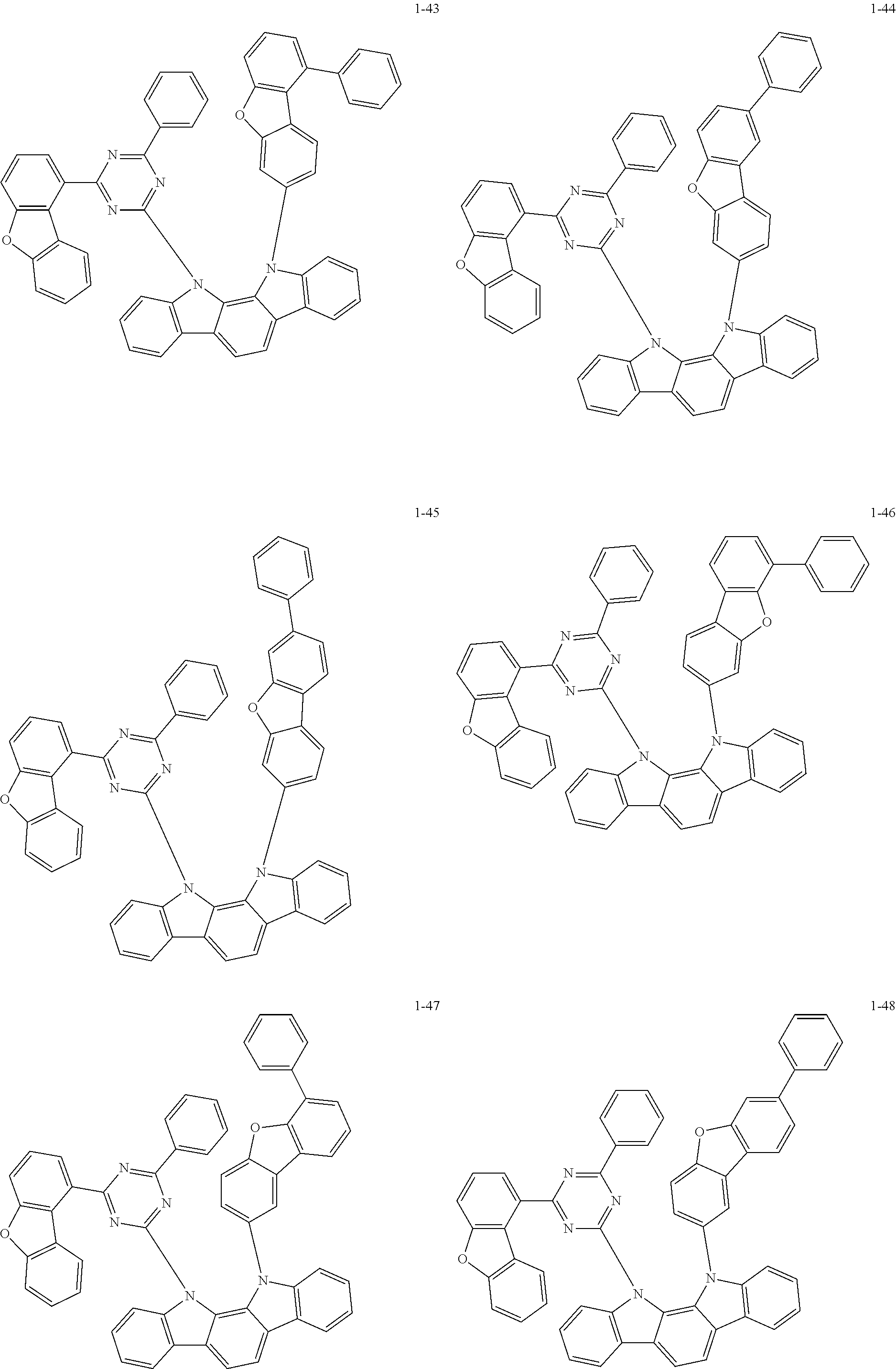

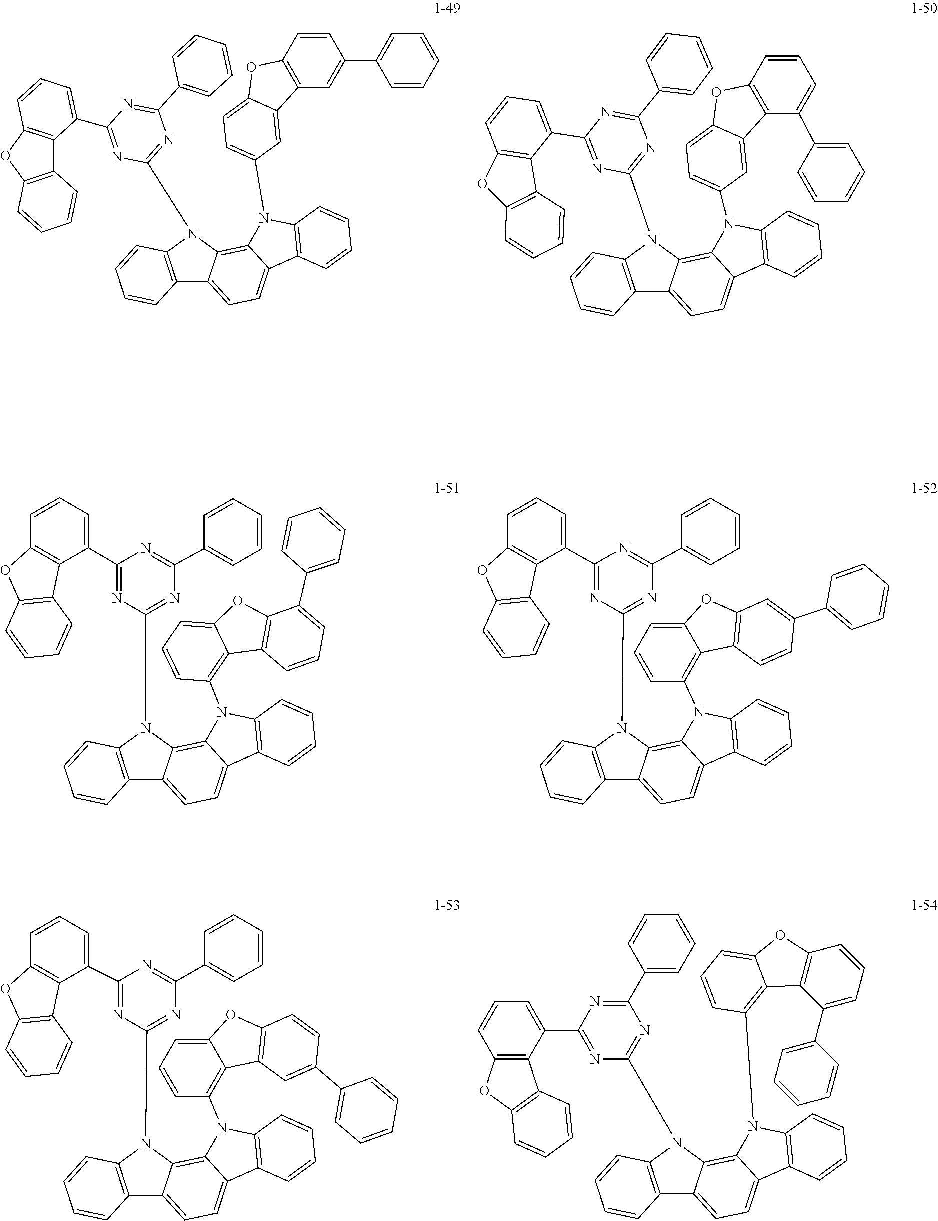

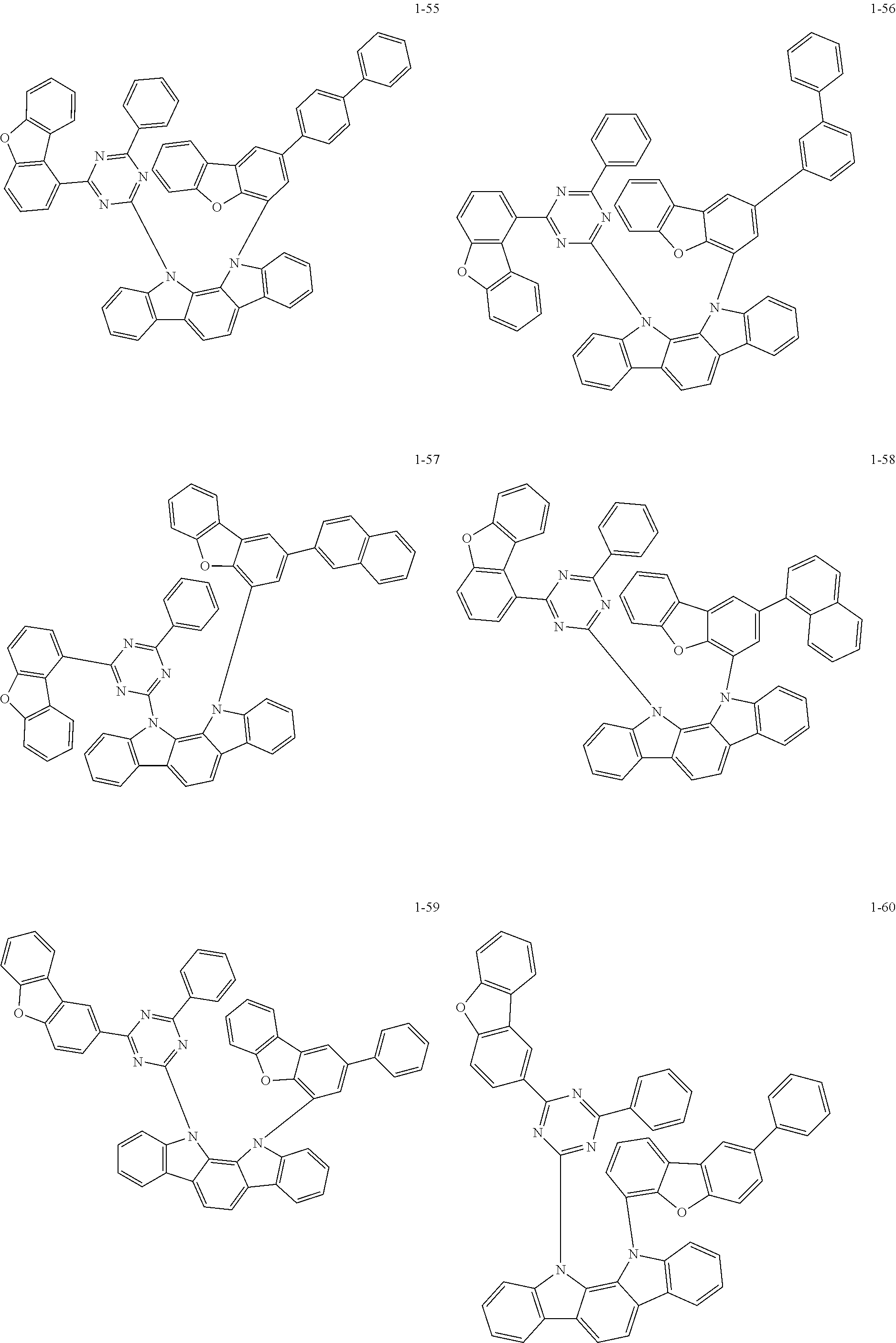

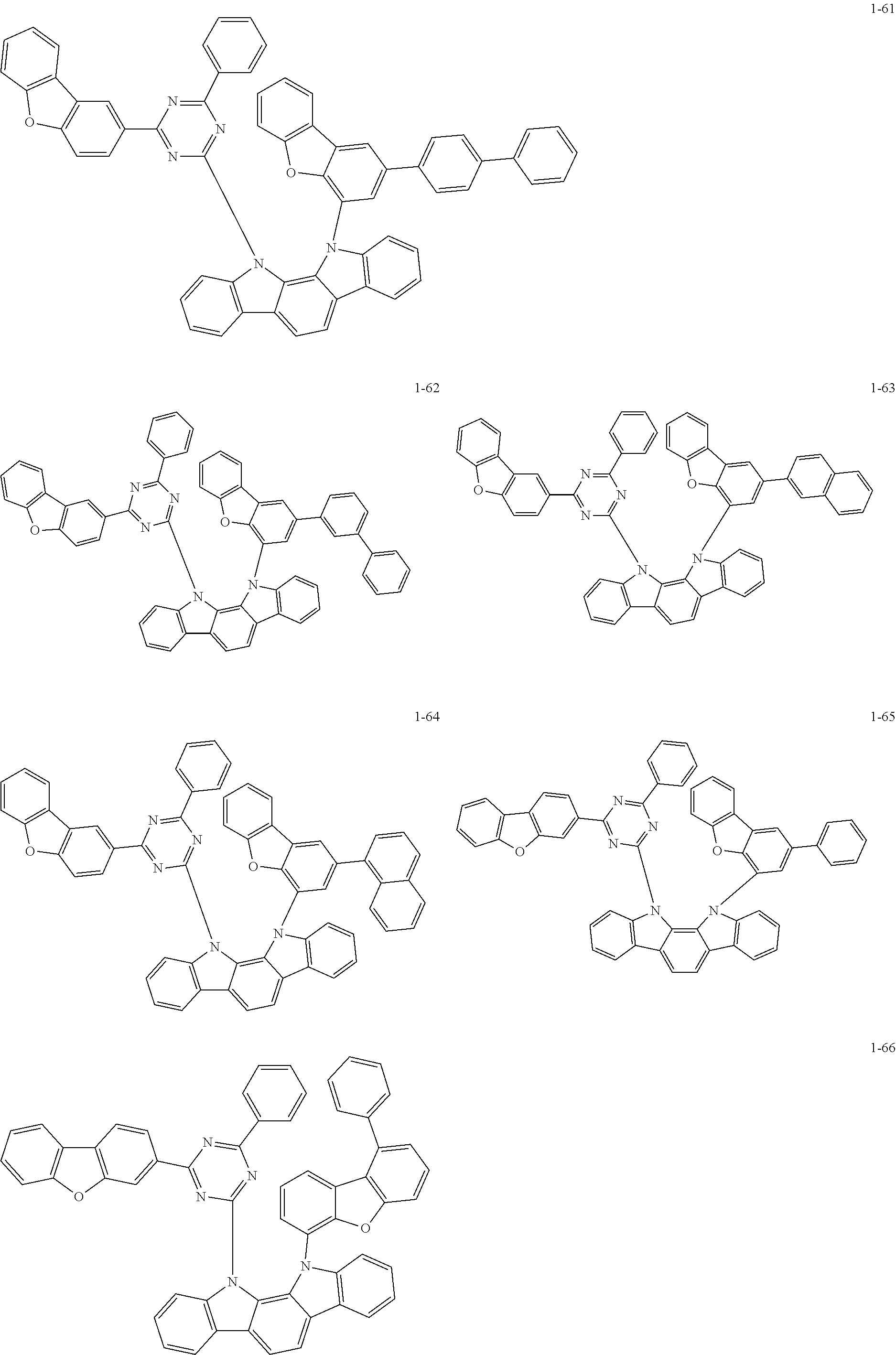

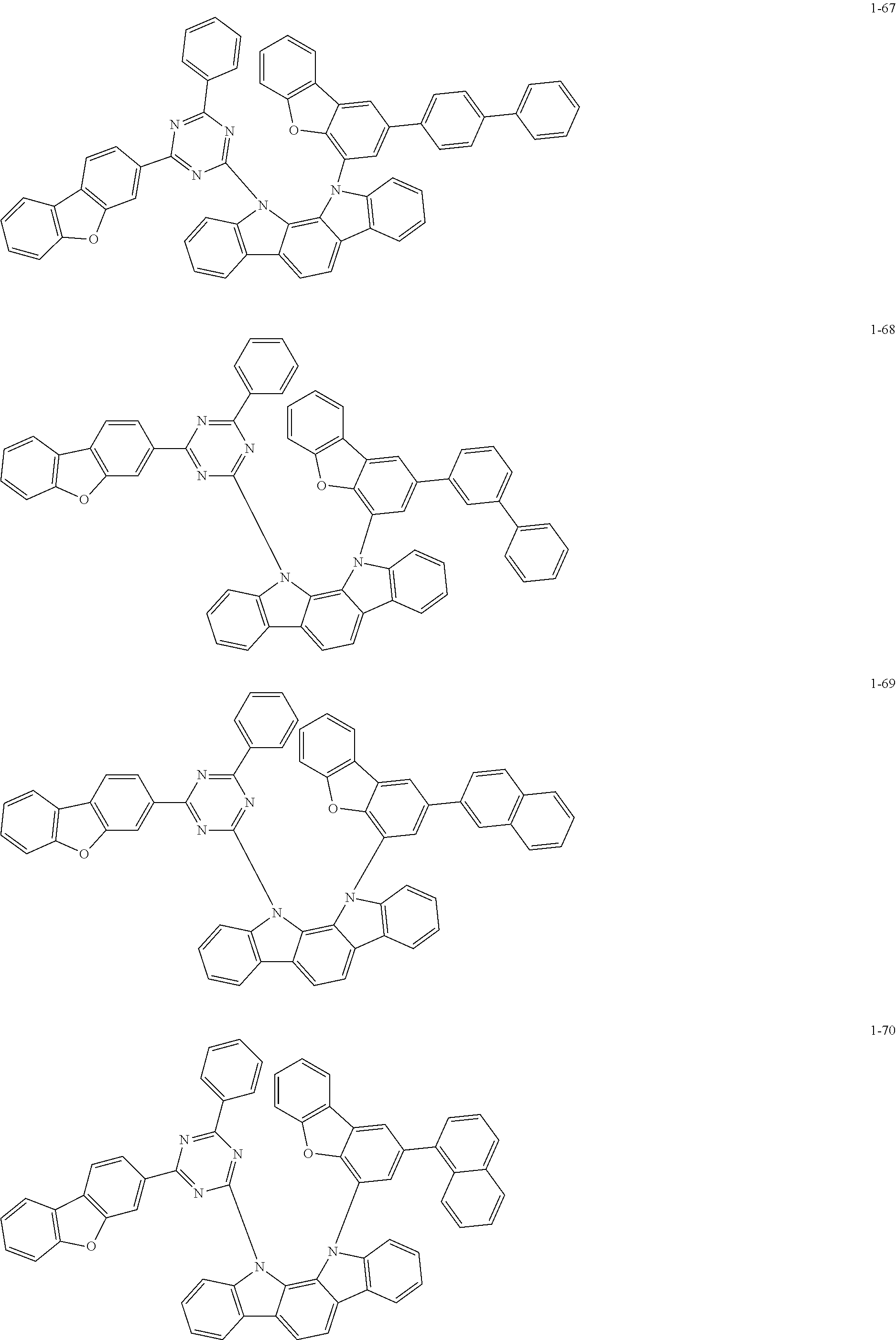

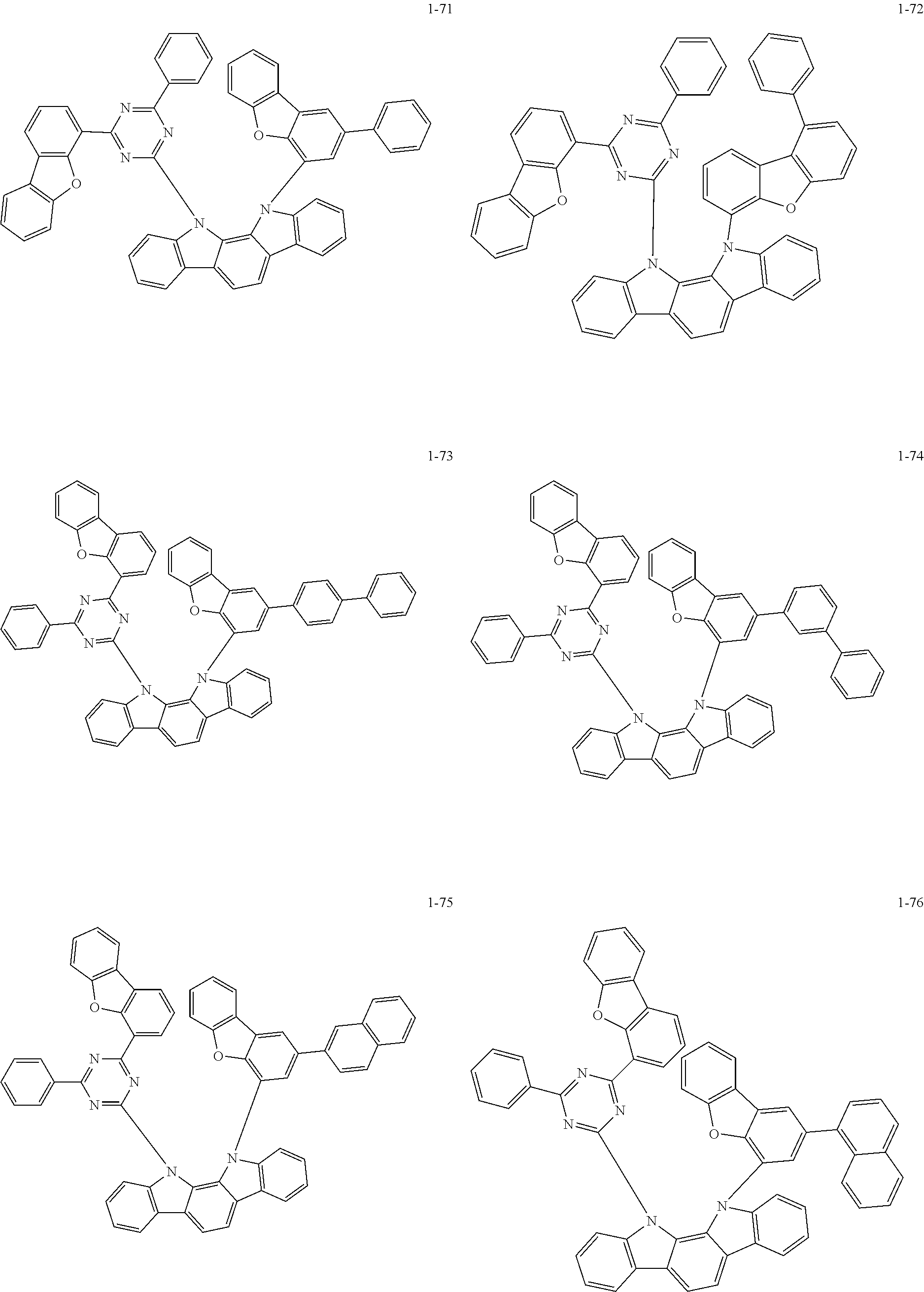

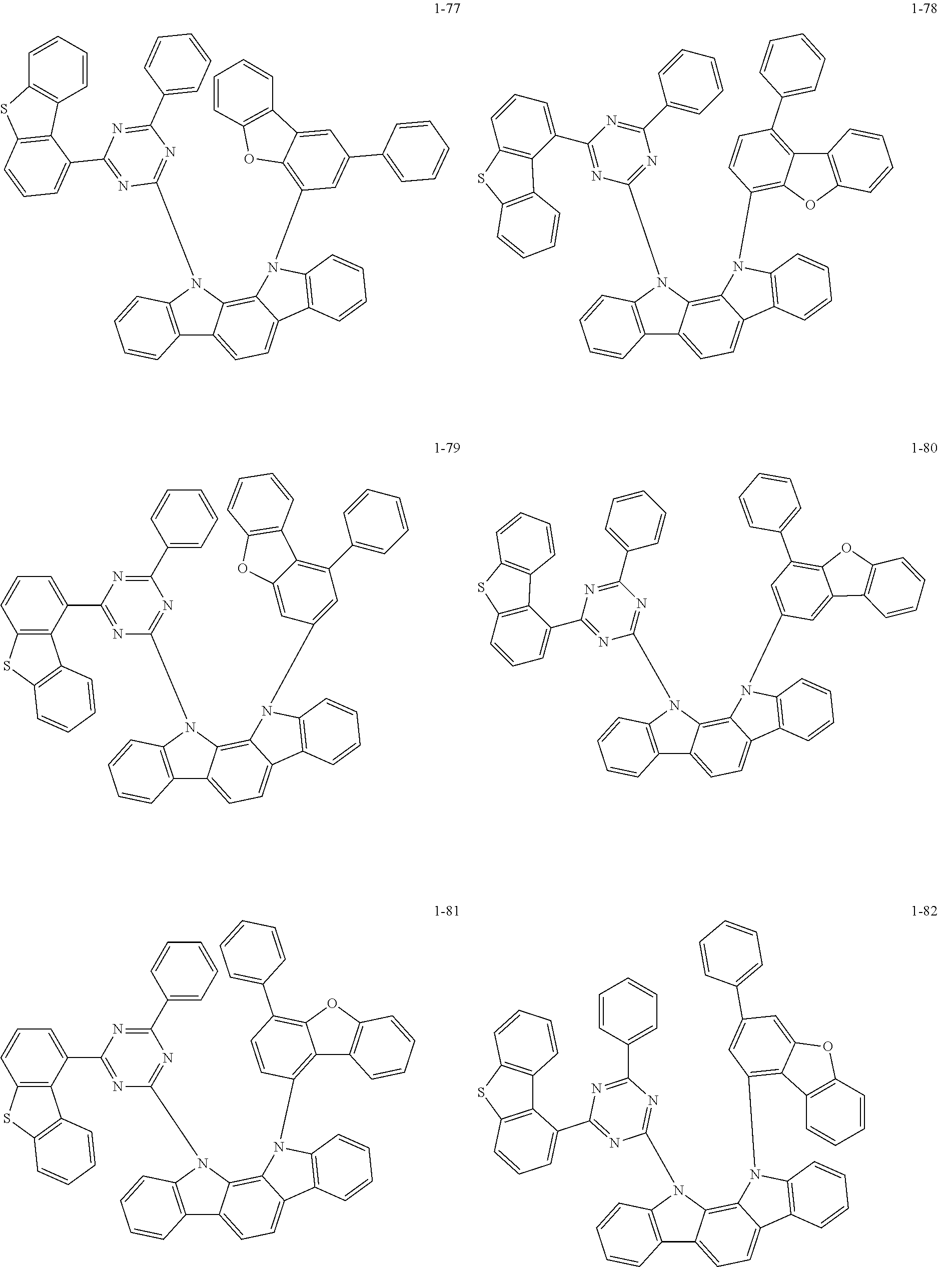

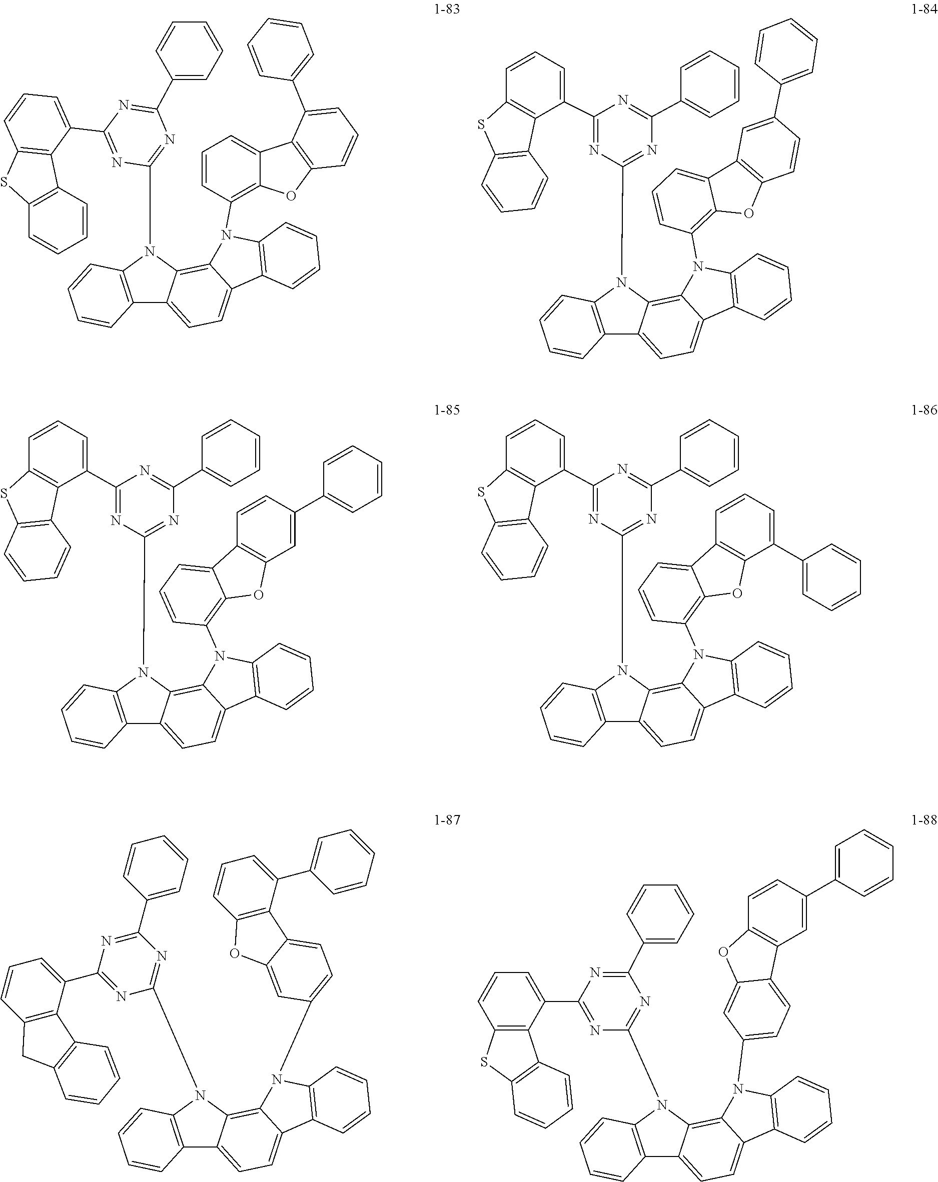

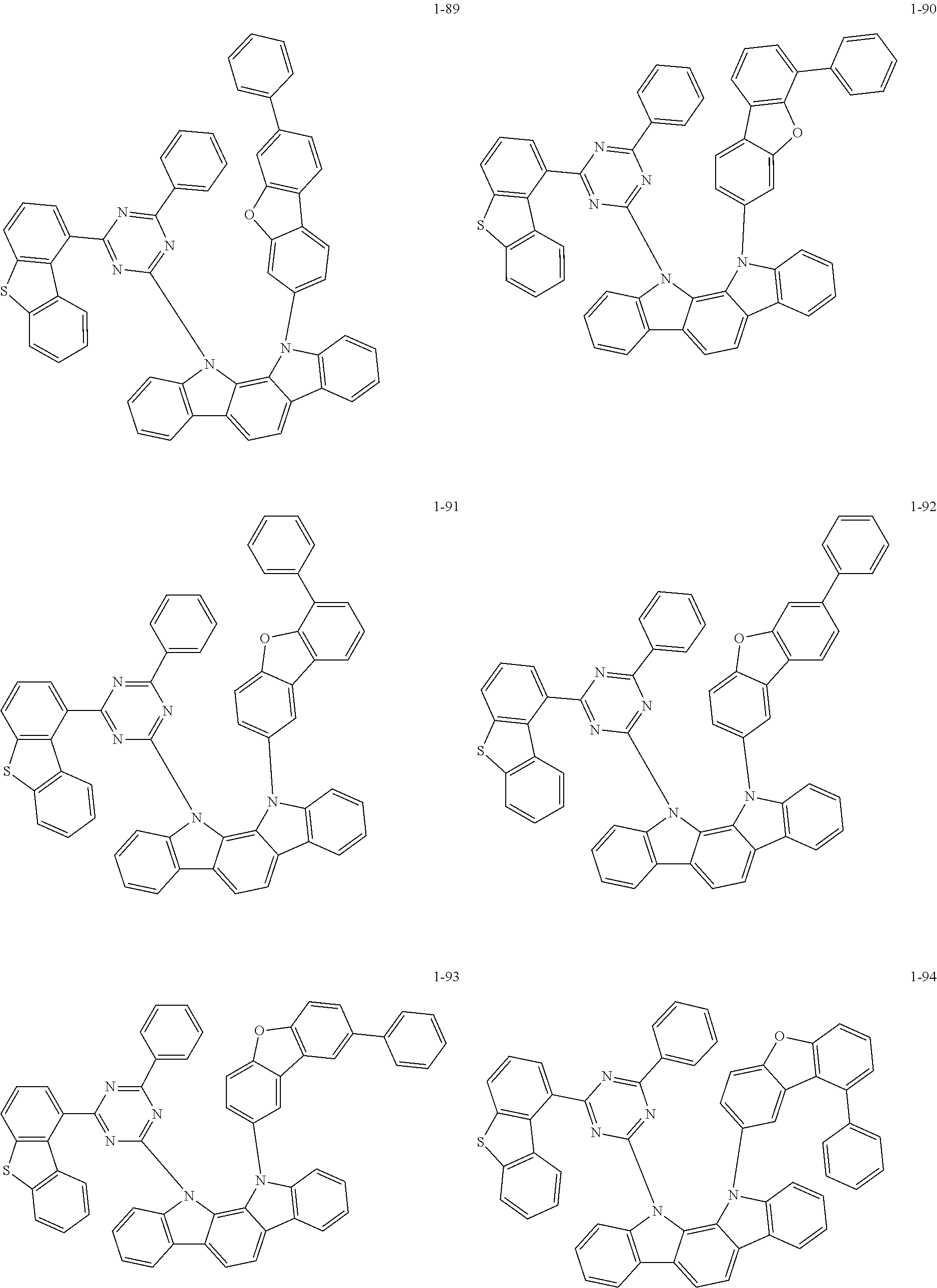

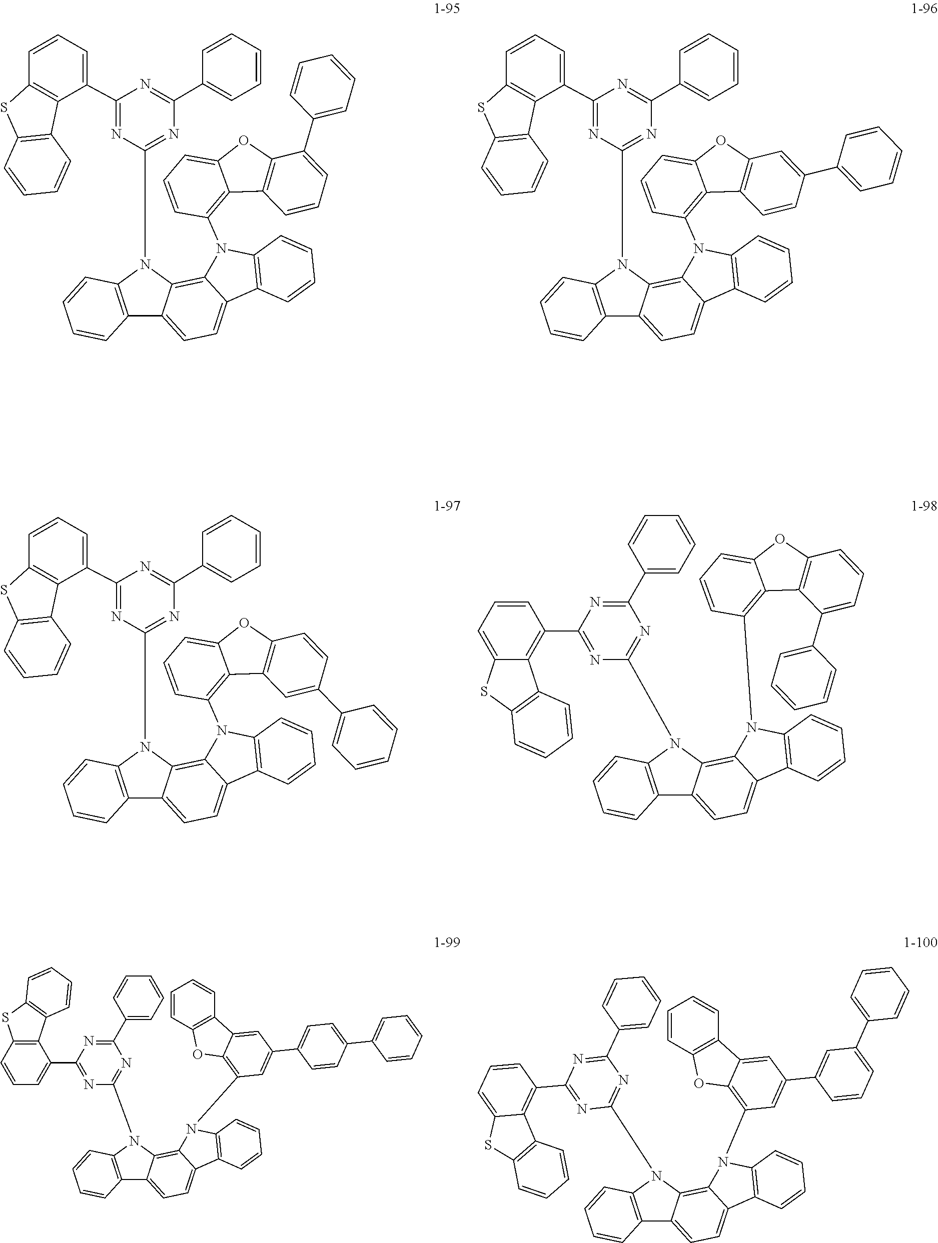

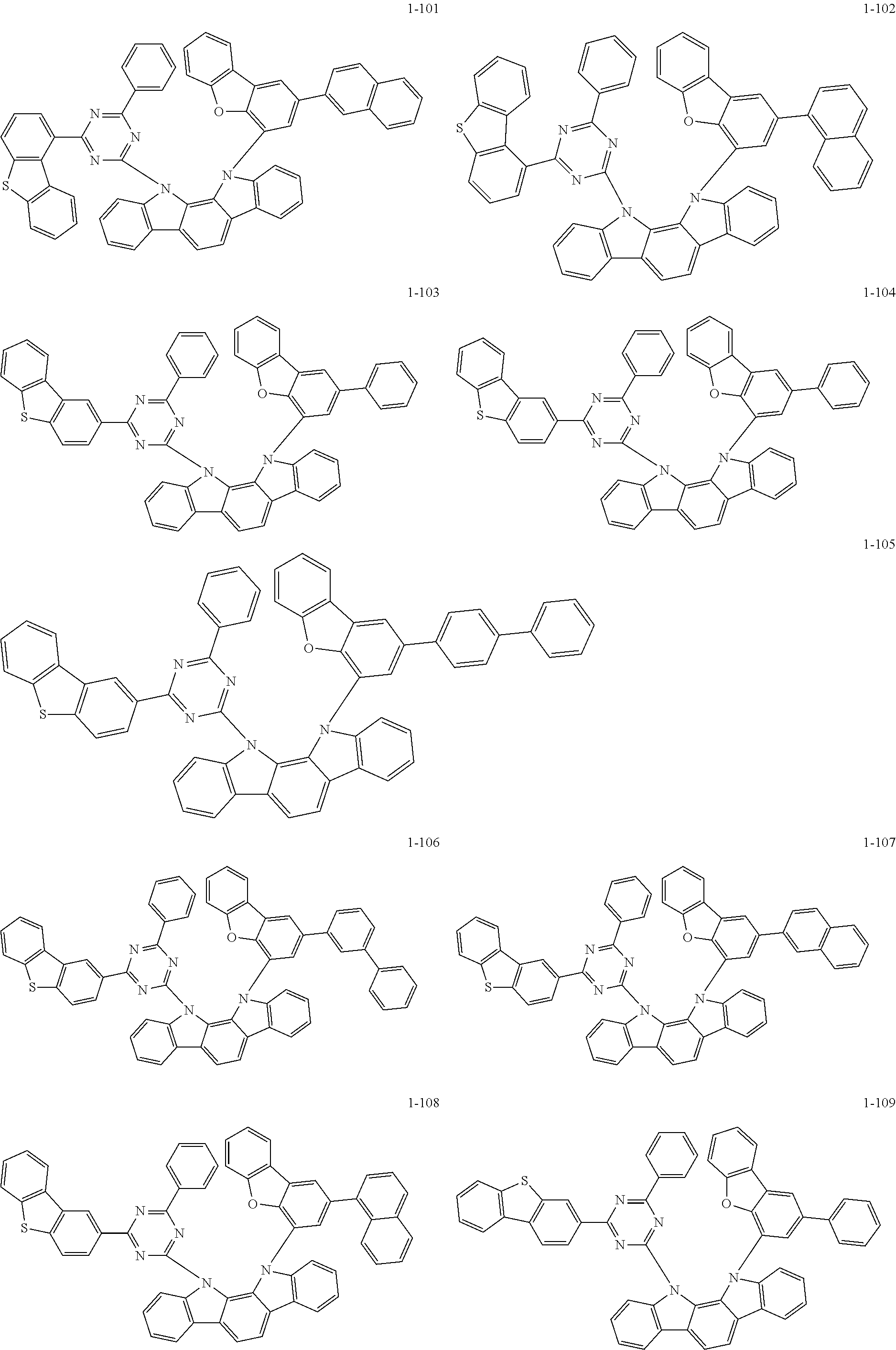

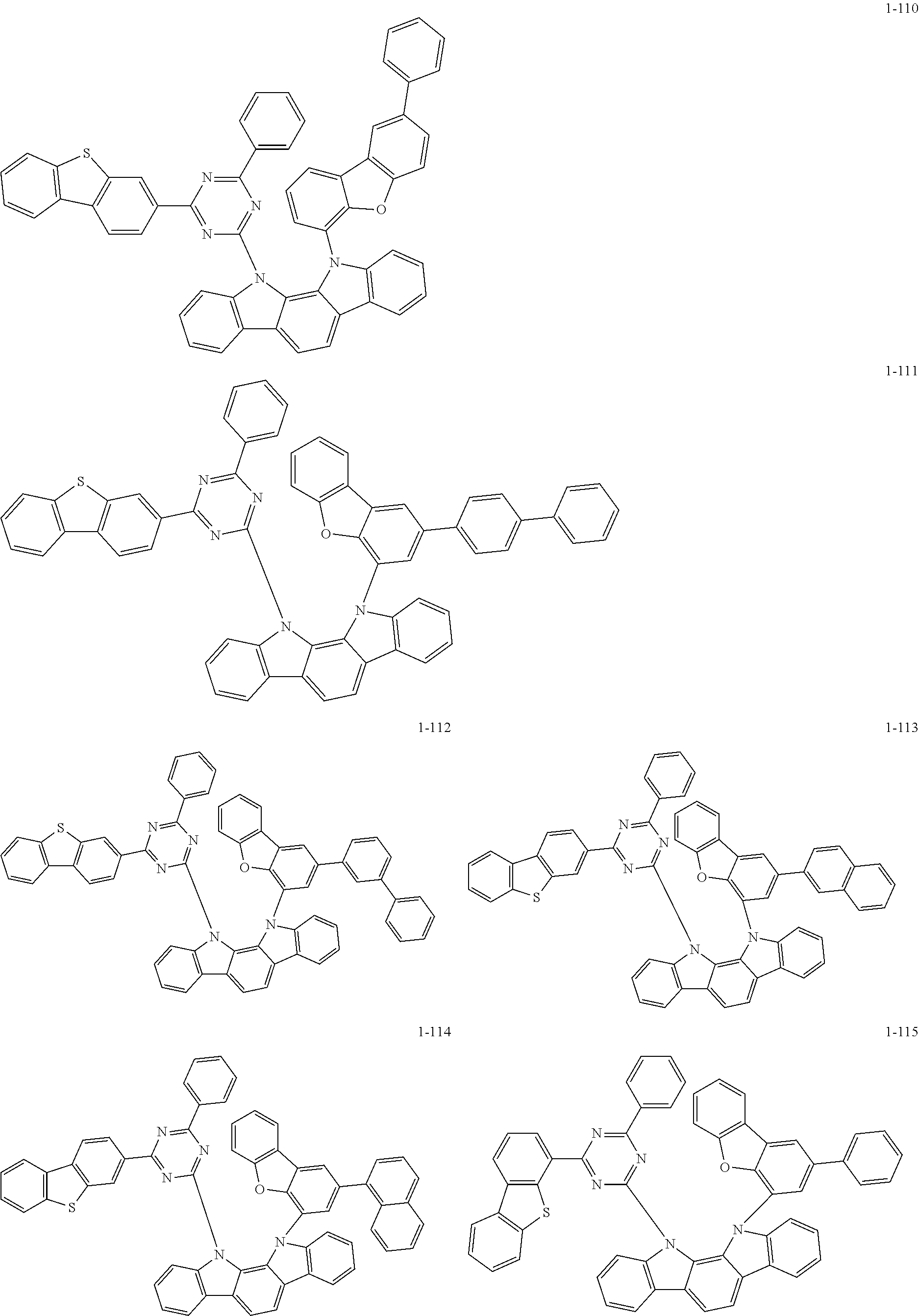

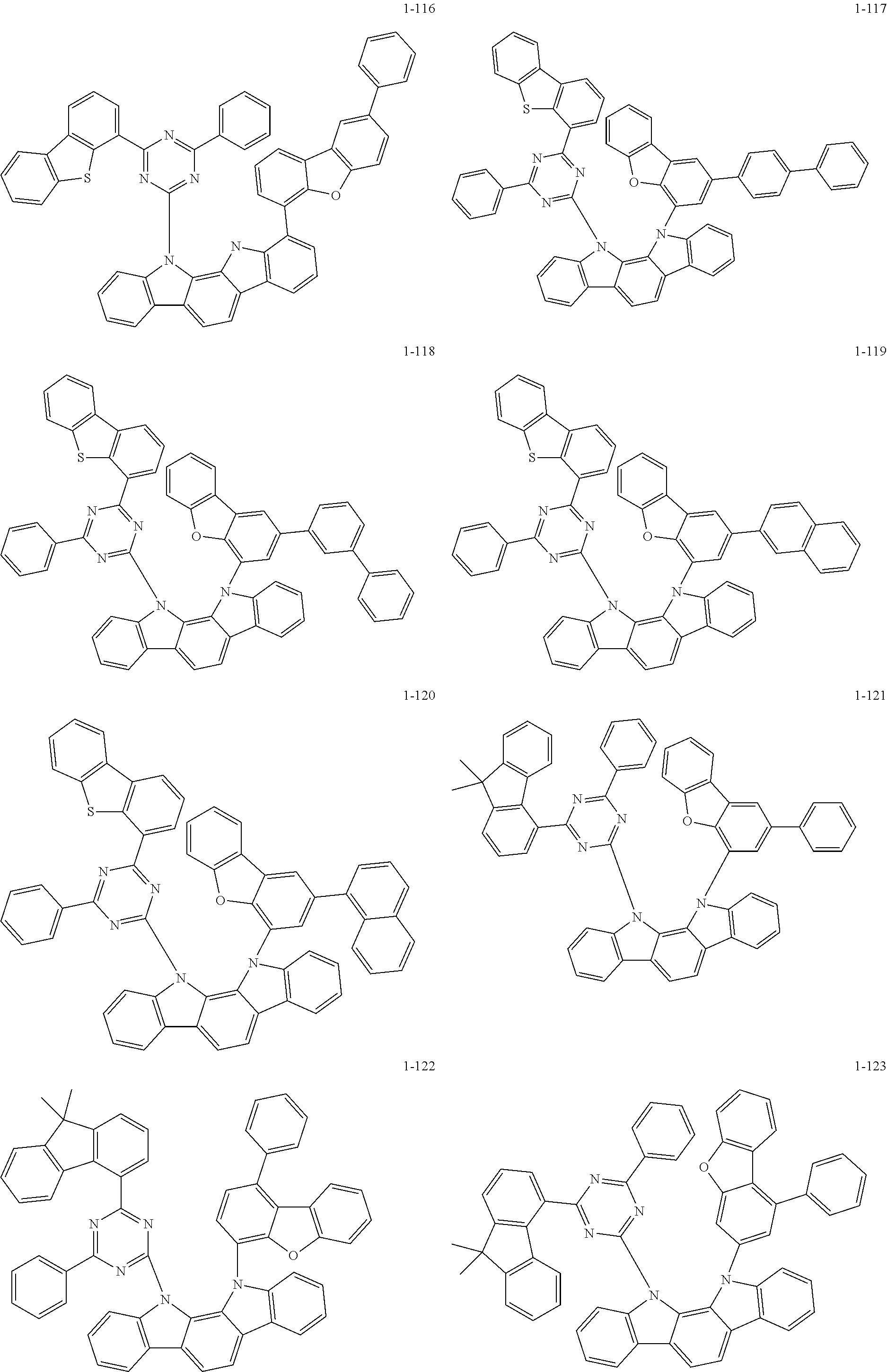

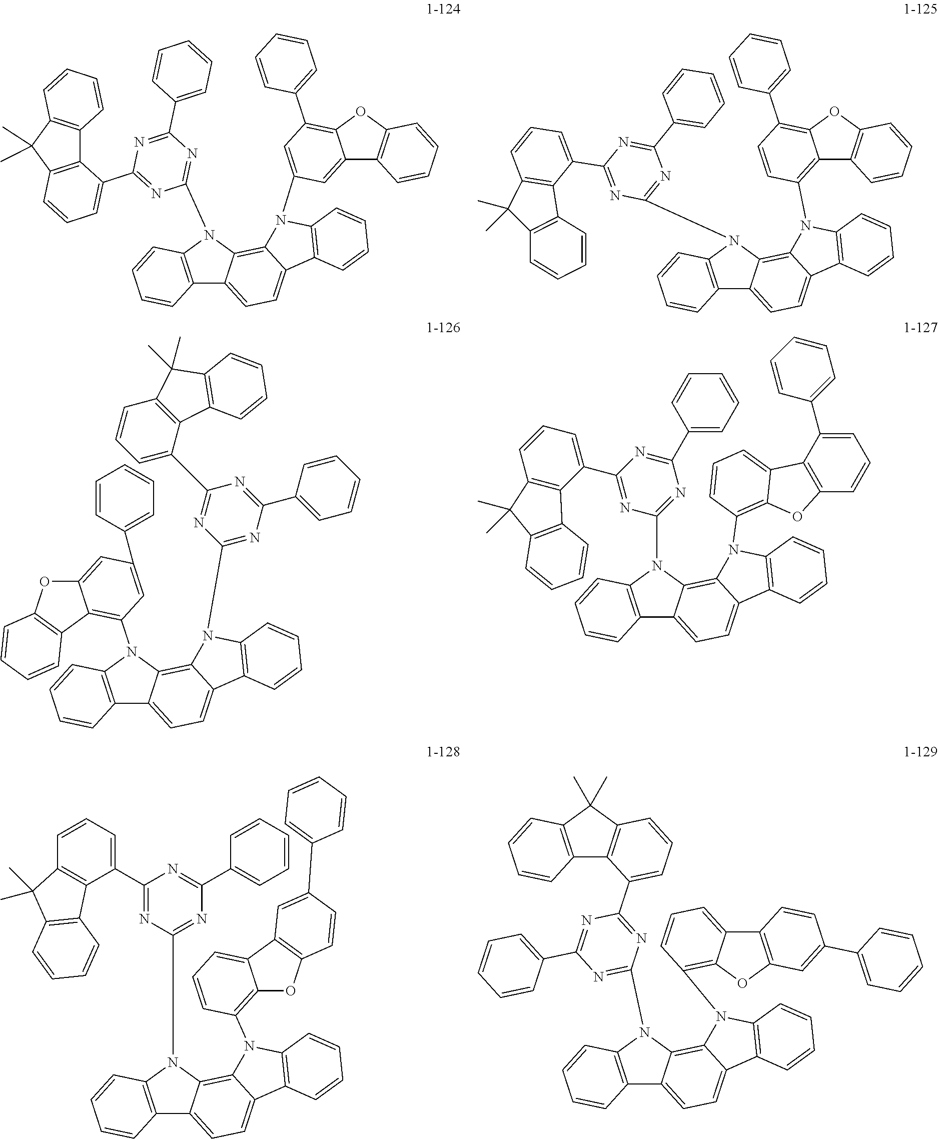

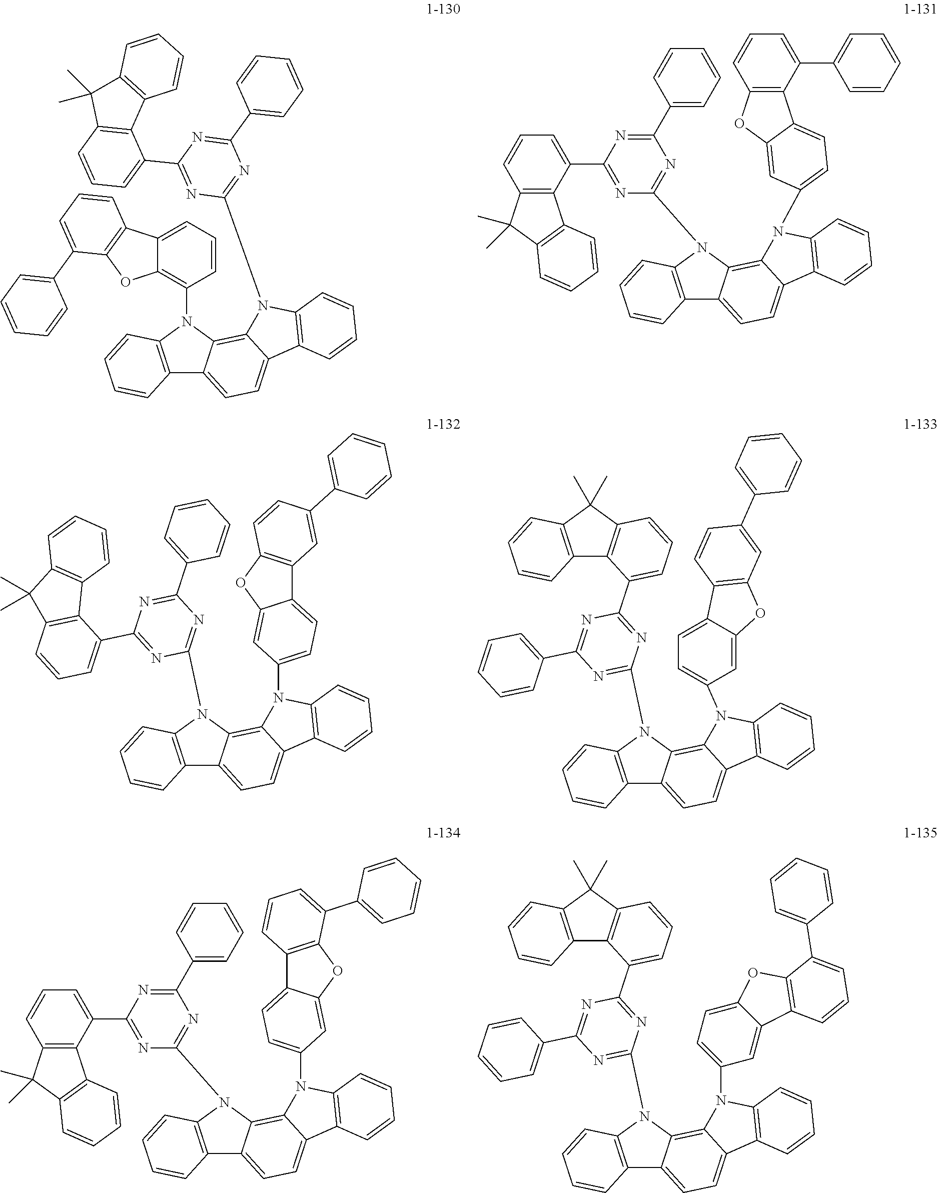

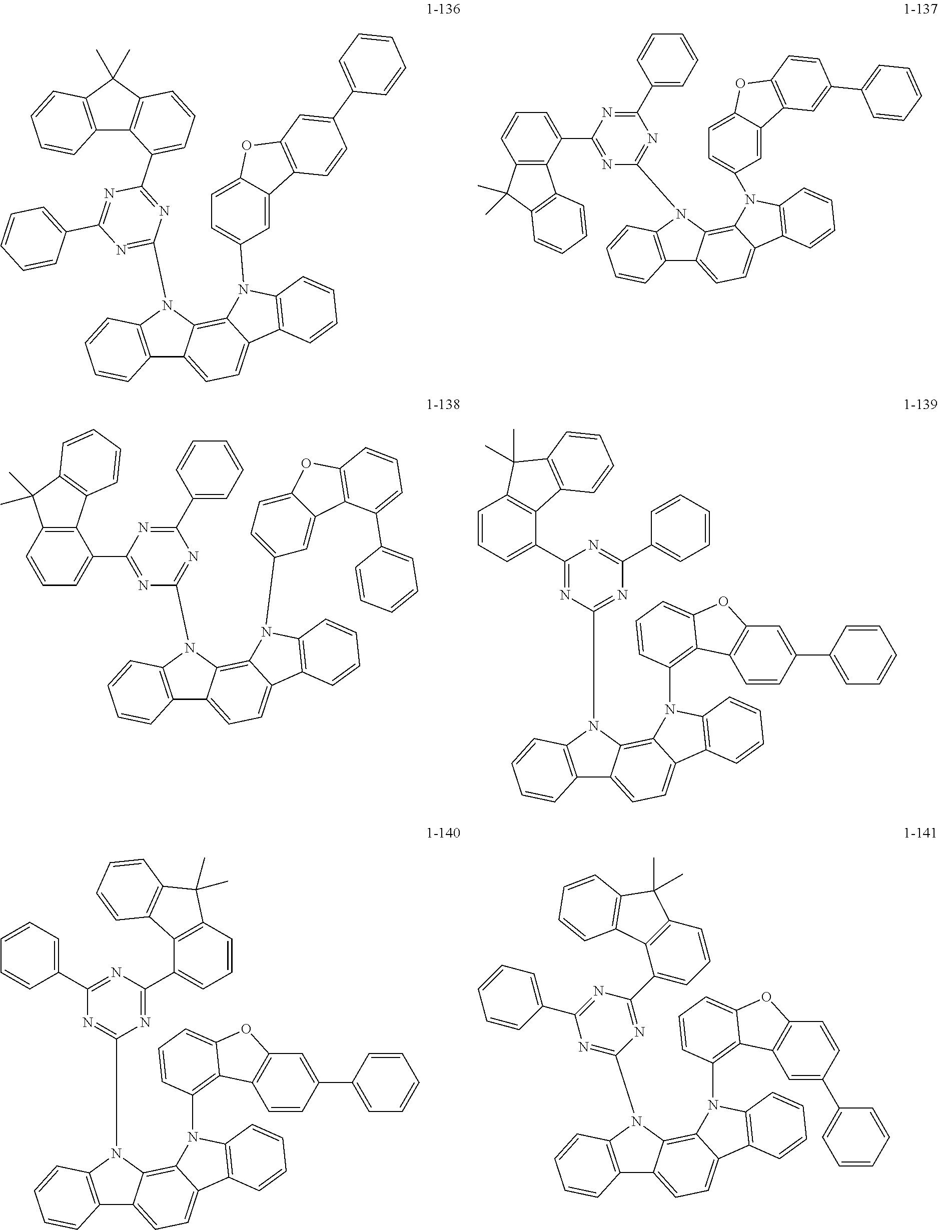

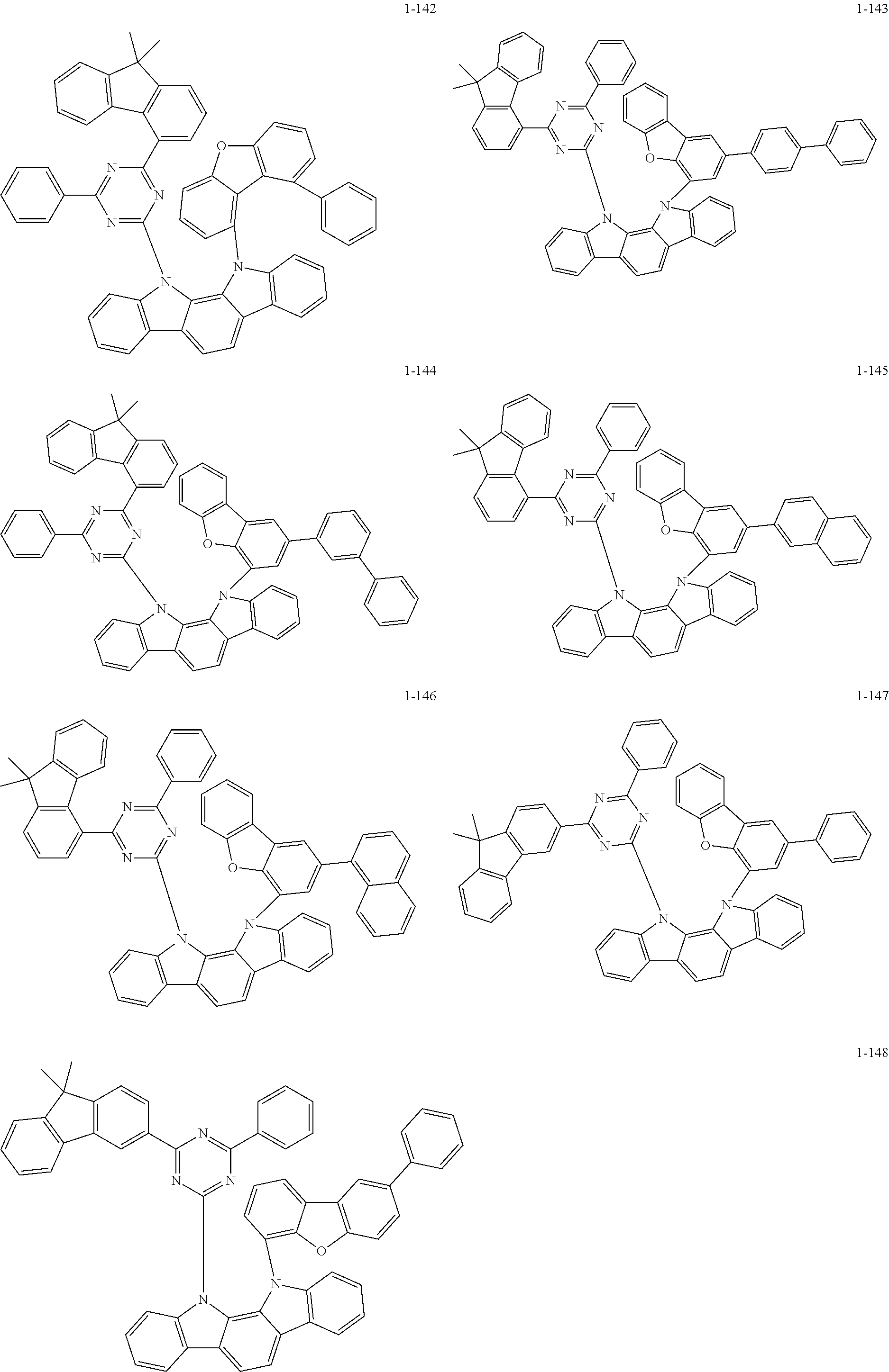

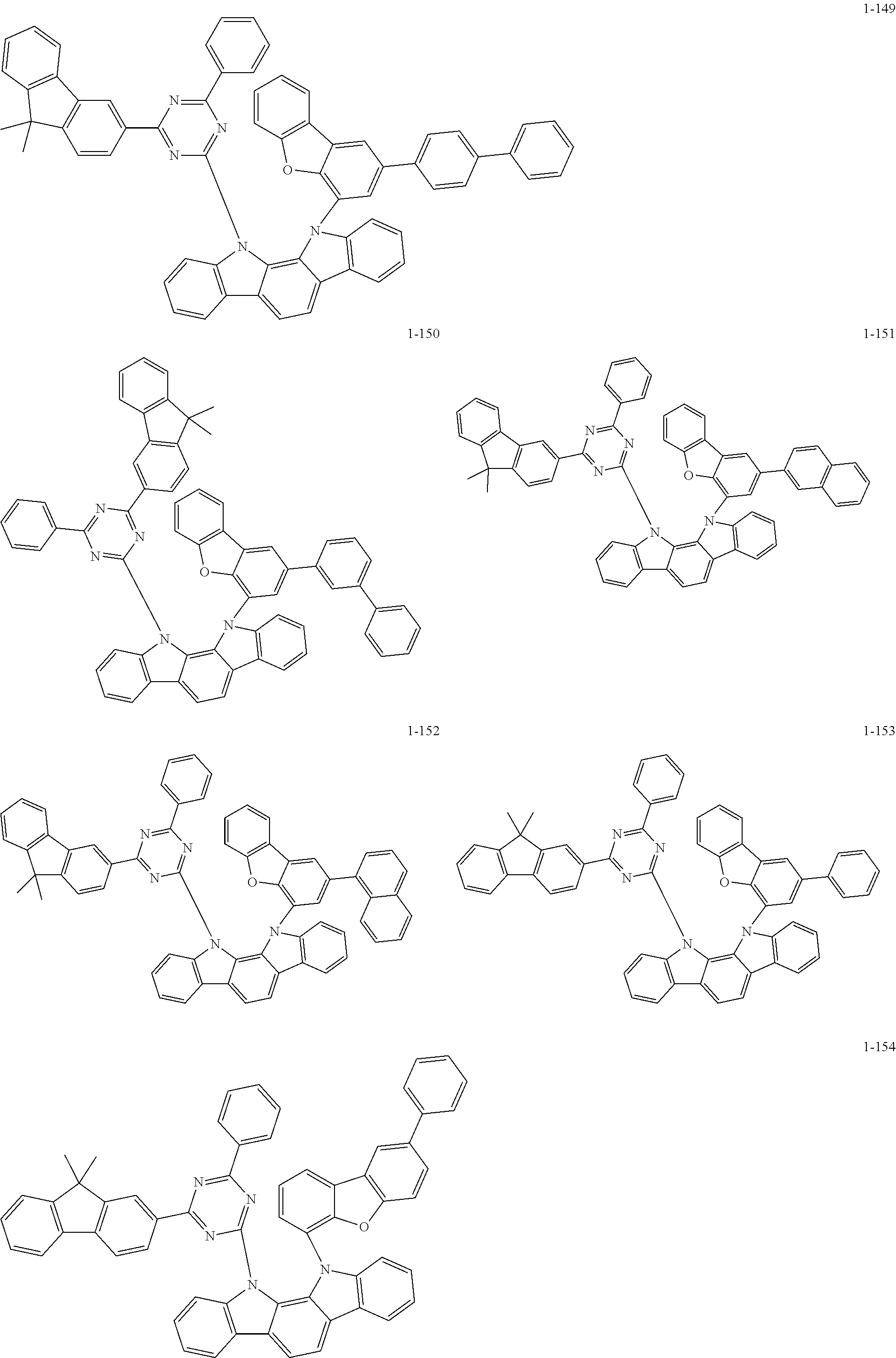

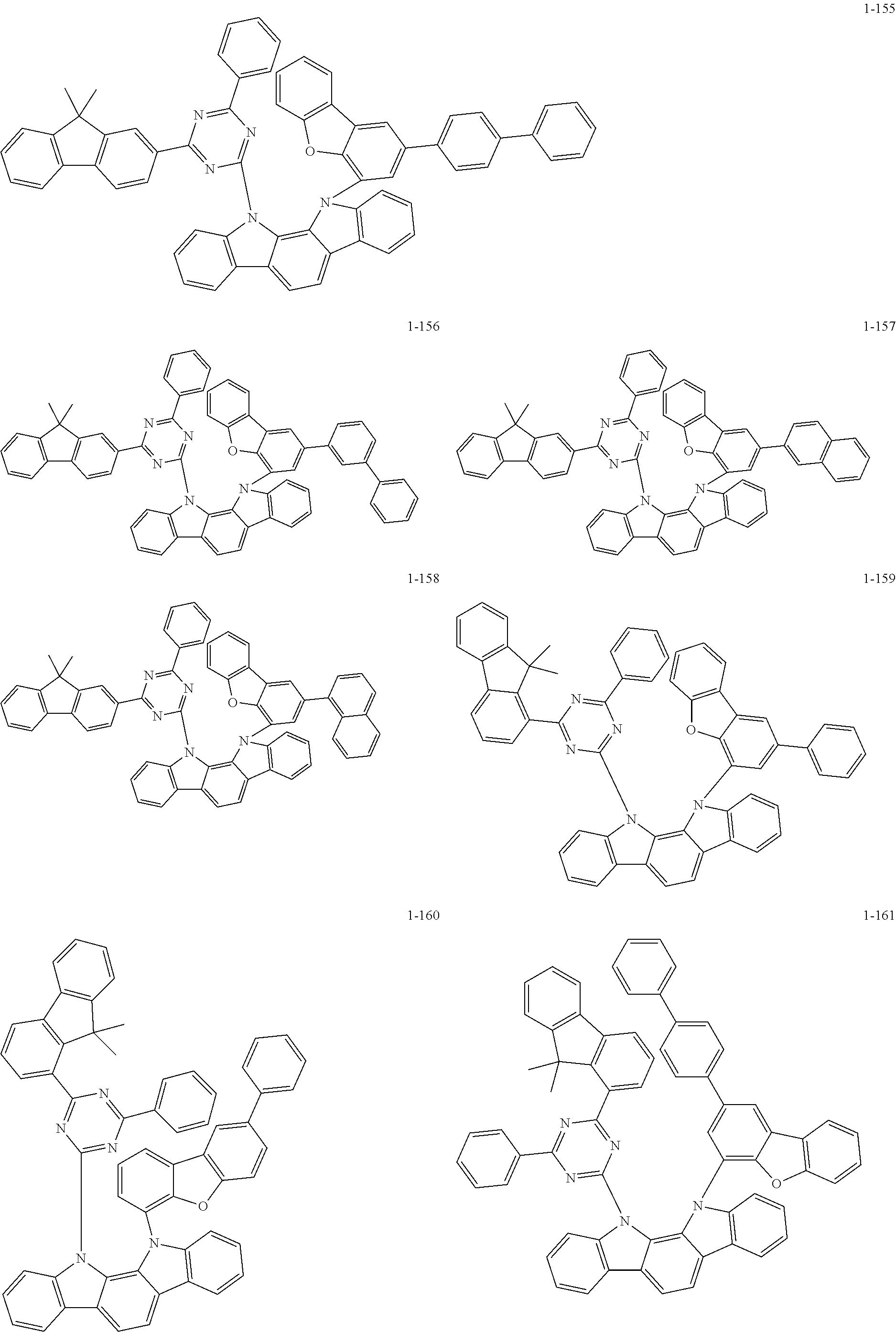

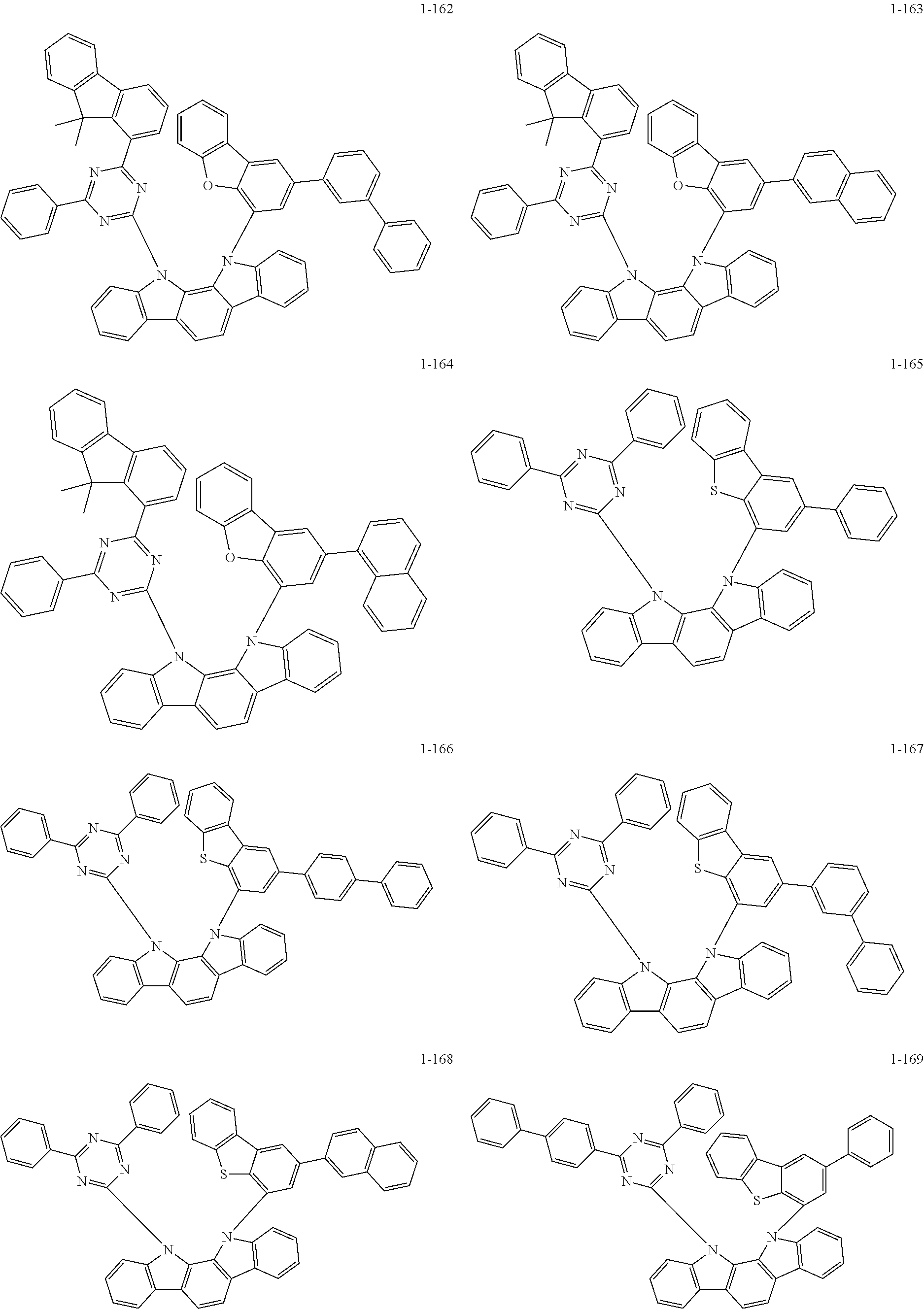

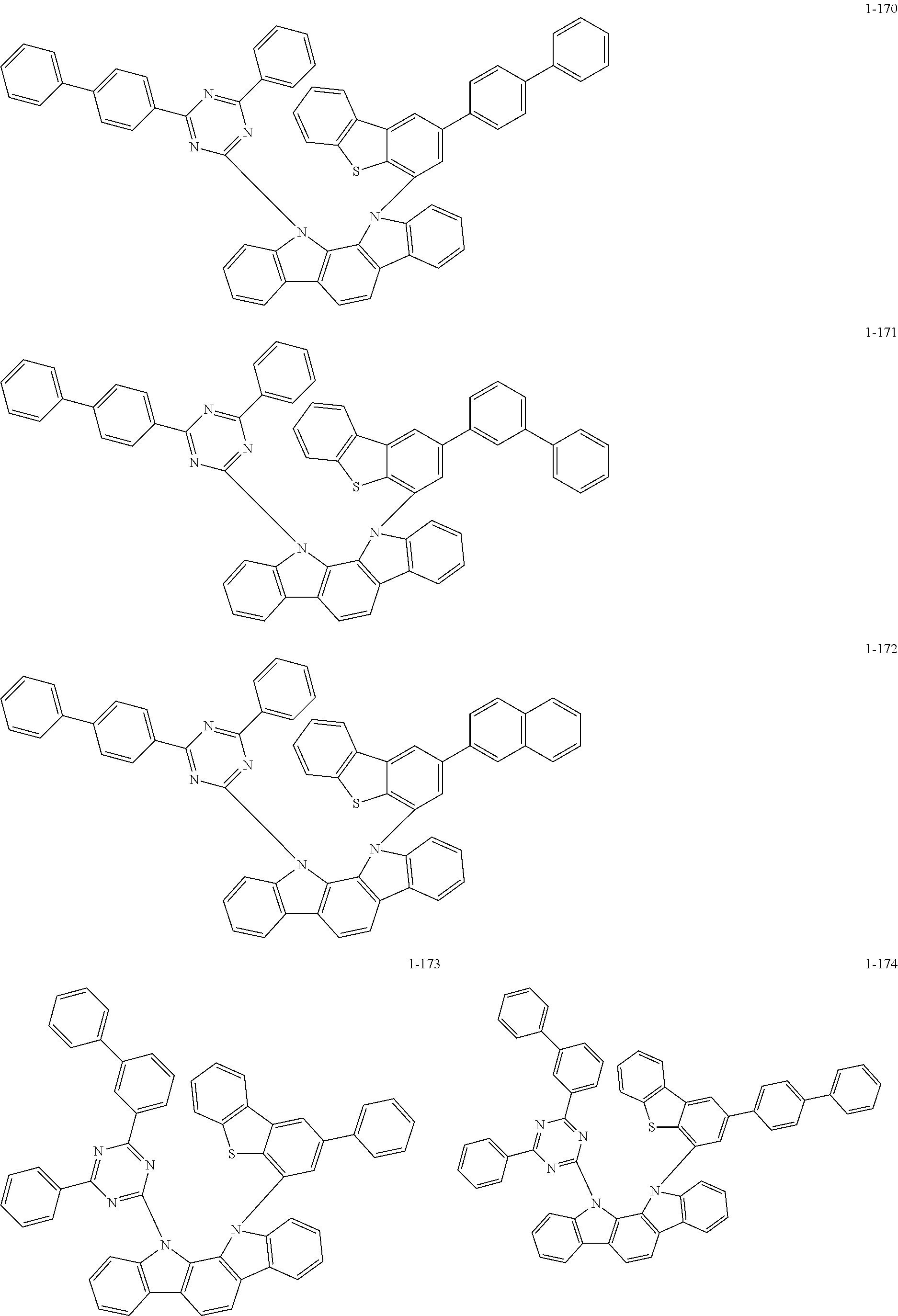

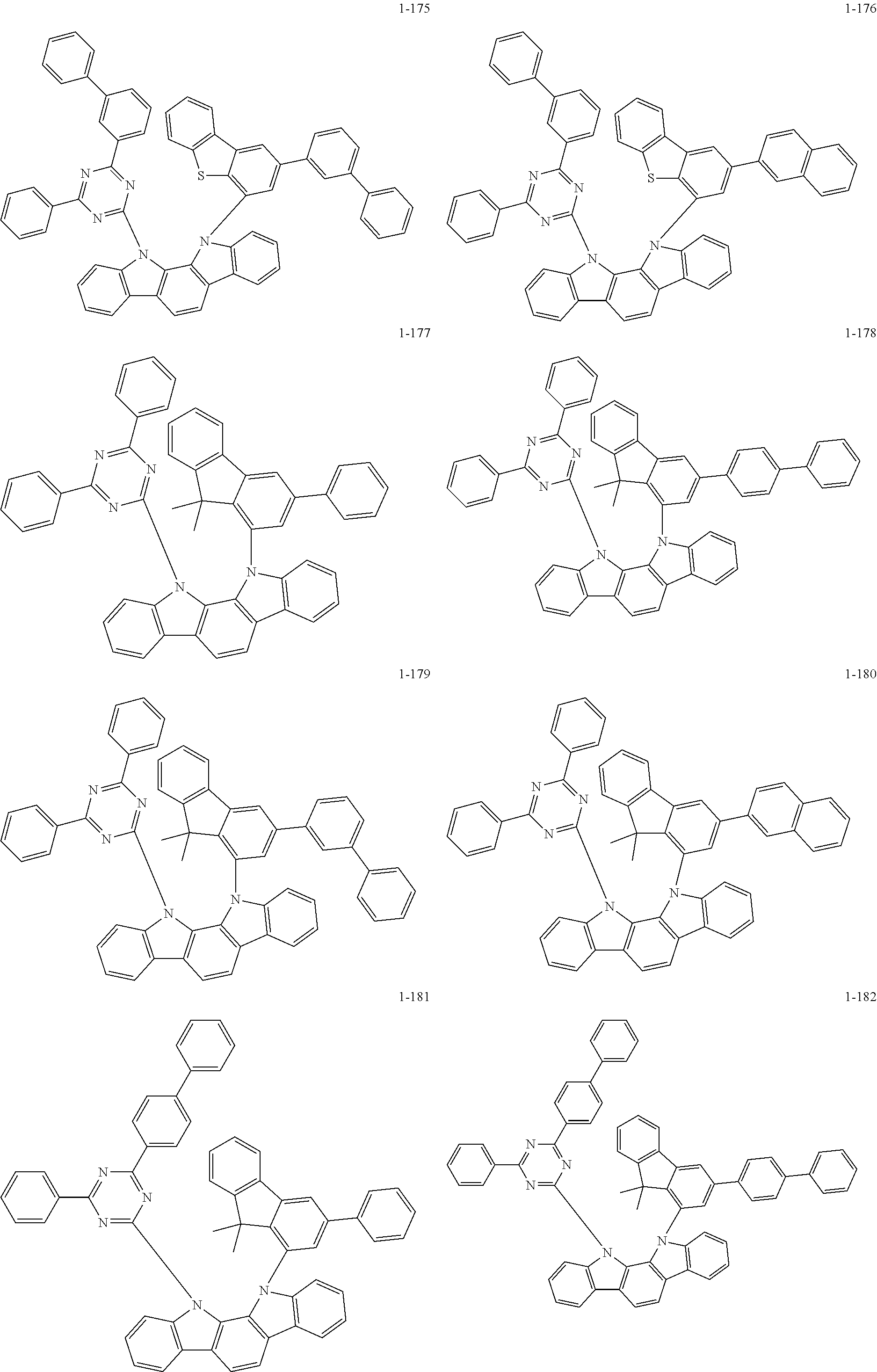

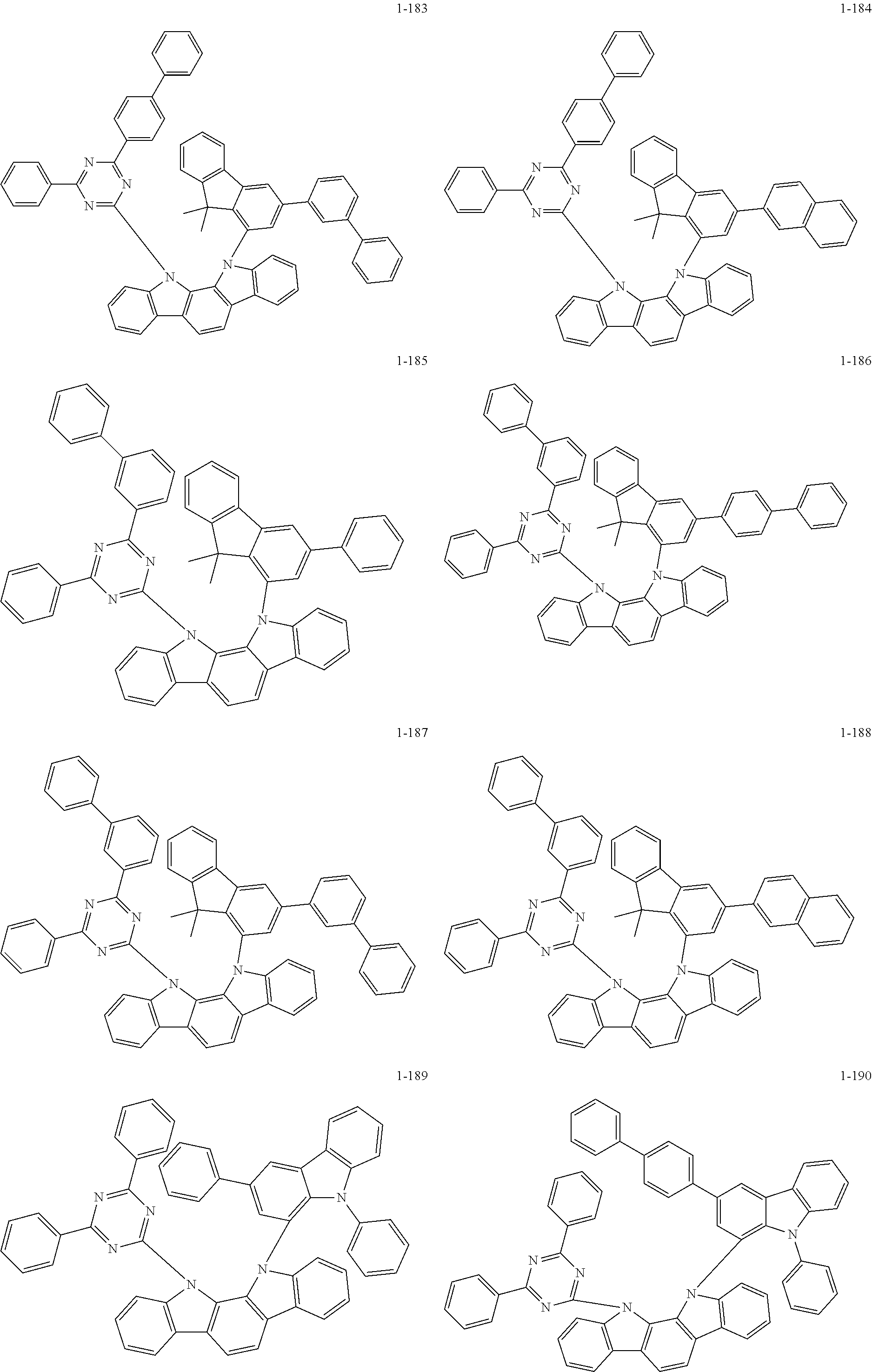

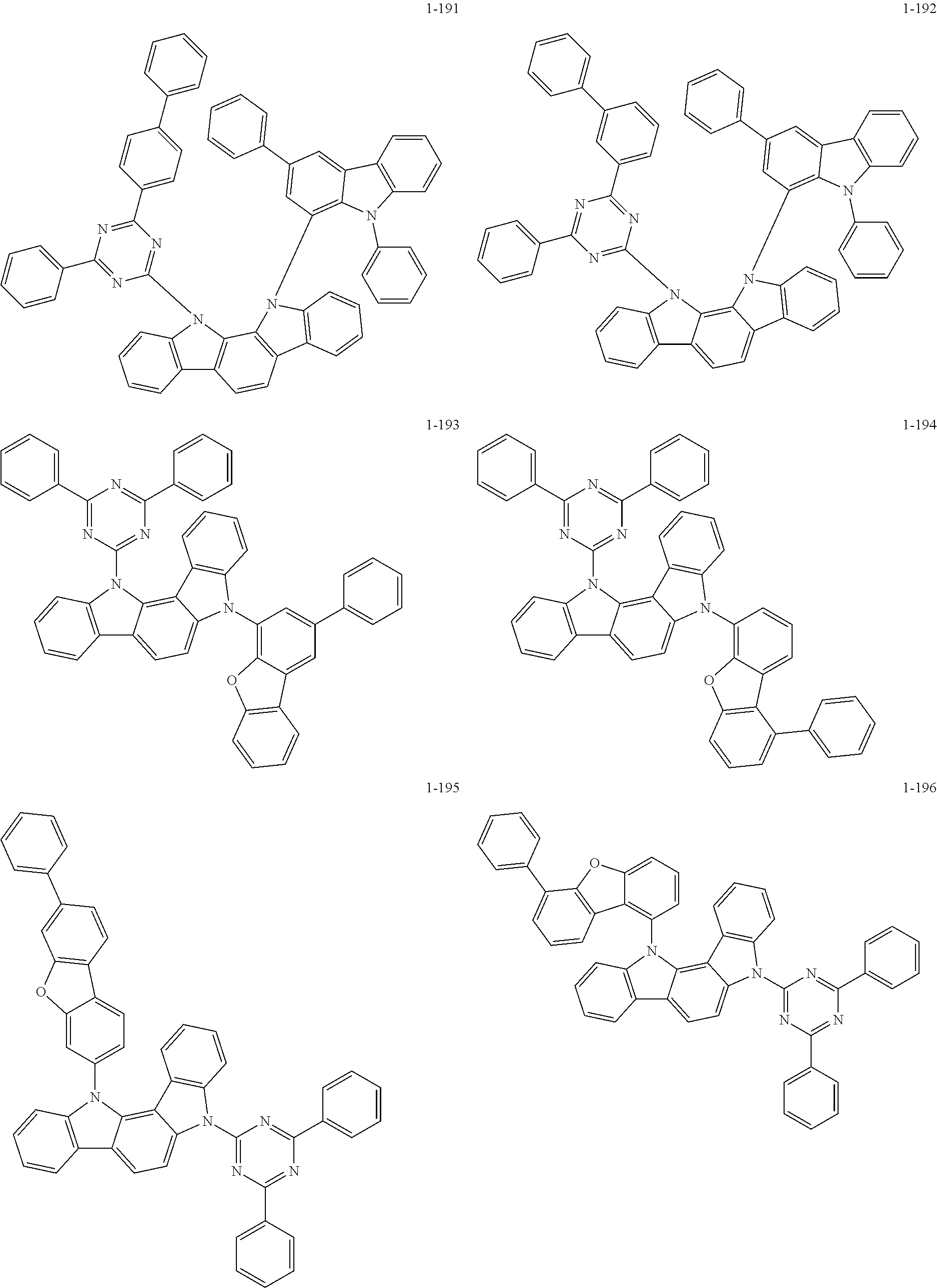

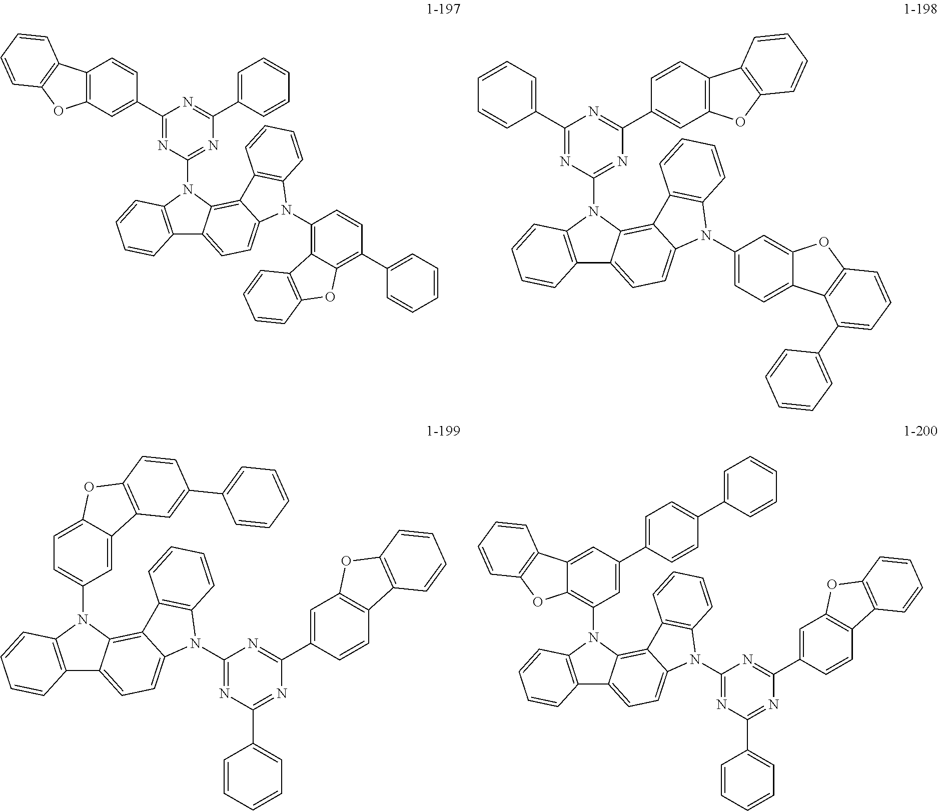

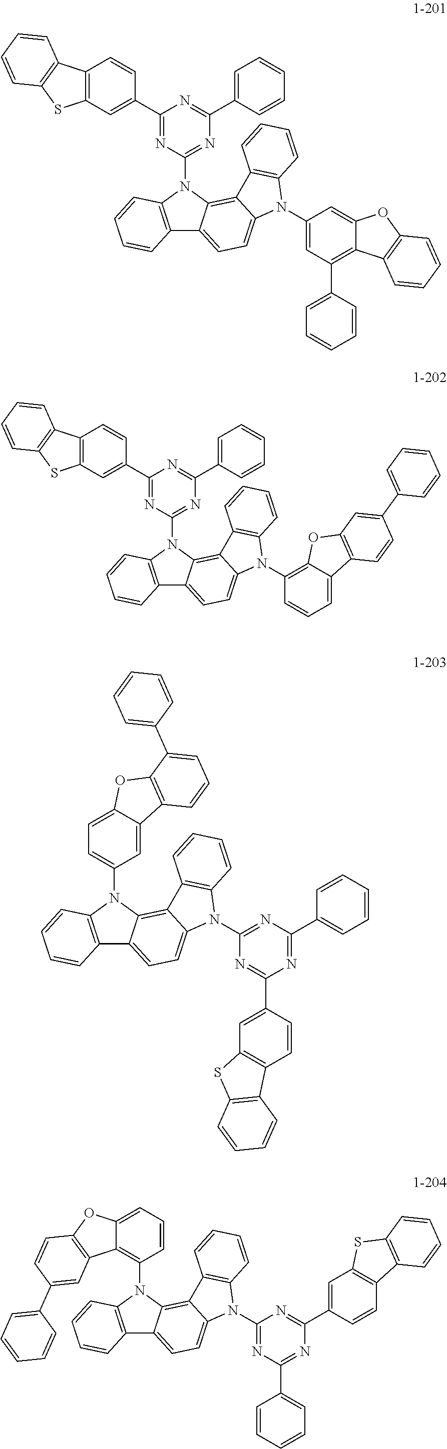 [SMILES: c1ccc(-c2nc(-c3ccc4c(c3)sc3ccccc34)nc(-n3c4ccccc4c4ccc5c(c6ccccc6n5-c5cc(-c6ccccc6)c6c(c5)oc5ccccc56)c43)n2)cc1 ; c1ccc(-c2ccc3c(c2)oc2c(-n4c5ccccc5c5c4ccc4c6ccccc6n(-c6nc(-c7ccccc7)nc(-c7ccc8c(c7)sc7ccccc78)n6)c45)cccc23)cc1 ; c1ccc(-c2nc(-c3ccc4c(c3)sc3ccccc34)nc(-n3c4ccccc4c4c3ccc3c5ccccc5n(-c5ccc6oc7c(-c8ccccc8)cccc7c6c5)c34)n2)cc1 ; c1ccc(-c2ccc3oc4cccc(-n5c6ccccc6c6ccc7c(c8ccccc8n7-c7nc(-c8ccccc8)nc(-c8ccc9c(c8)sc8ccccc89)n7)c65)c4c3c2)cc1]

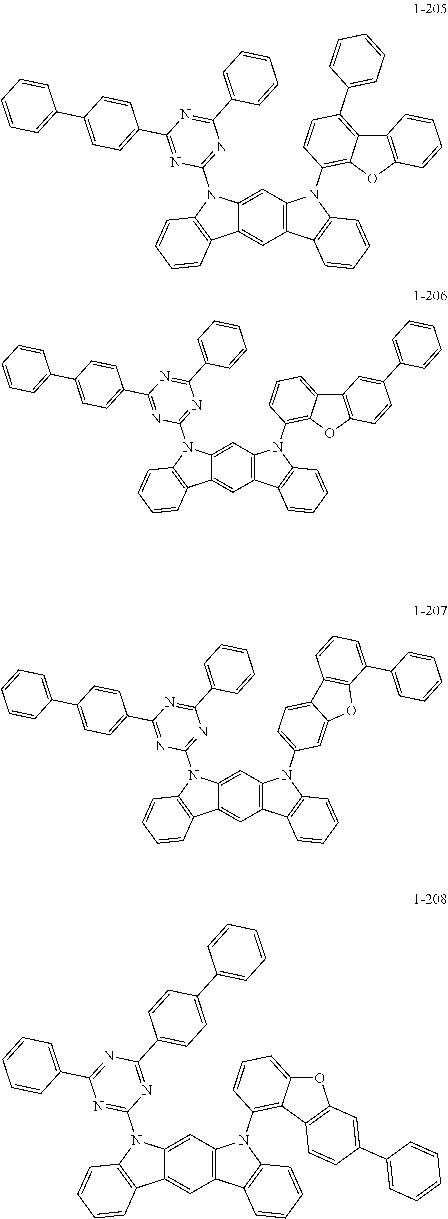 [SMILES: c1ccc(-c2ccc(-c3nc(-c4ccccc4)nc(-n4c5ccccc5c5cc6c7ccccc7n(-c7ccc(-c8ccccc8)c8c7oc7ccccc78)c6cc54)n3)cc2)cc1 ; c1ccc(-c2ccc(-c3nc(-c4ccccc4)nc(-n4c5ccccc5c5cc6c7ccccc7n(-c7cccc8c7oc7ccc(-c9ccccc9)cc78)c6cc54)n3)cc2)cc1 ; c1ccc(-c2ccc(-c3nc(-c4ccccc4)nc(-n4c5ccccc5c5cc6c7ccccc7n(-c7ccc8c(c7)oc7c(-c9ccccc9)cccc78)c6cc54)n3)cc2)cc1 ; c1ccc(-c2ccc(-c3nc(-c4ccccc4)nc(-n4c5ccccc5c5cc6c7ccccc7n(-c7cccc8oc9cc(-c%10ccccc%10)ccc9c78)c6cc54)n3)cc2)cc1]

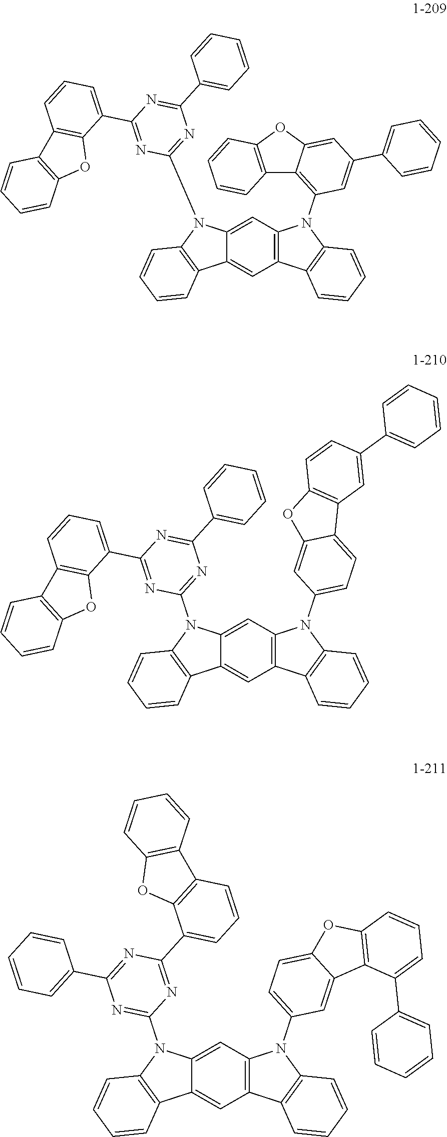 [SMILES: c1ccc(-c2cc(-n3c4ccccc4c4cc5c6ccccc6n(-c6nc(-c7ccccc7)nc(-c7cccc8c7oc7ccccc78)n6)c5cc43)c3c(c2)oc2ccccc23)cc1 ; c1ccc(-c2ccc3oc4cc(-n5c6ccccc6c6cc7c8ccccc8n(-c8nc(-c9ccccc9)nc(-c9cccc%10c9oc9ccccc9%10)n8)c7cc65)ccc4c3c2)cc1 ; c1ccc(-c2nc(-c3cccc4c3oc3ccccc34)nc(-n3c4ccccc4c4cc5c6ccccc6n(-c6ccc7oc8cccc(-c9ccccc9)c8c7c6)c5cc43)n2)cc1]

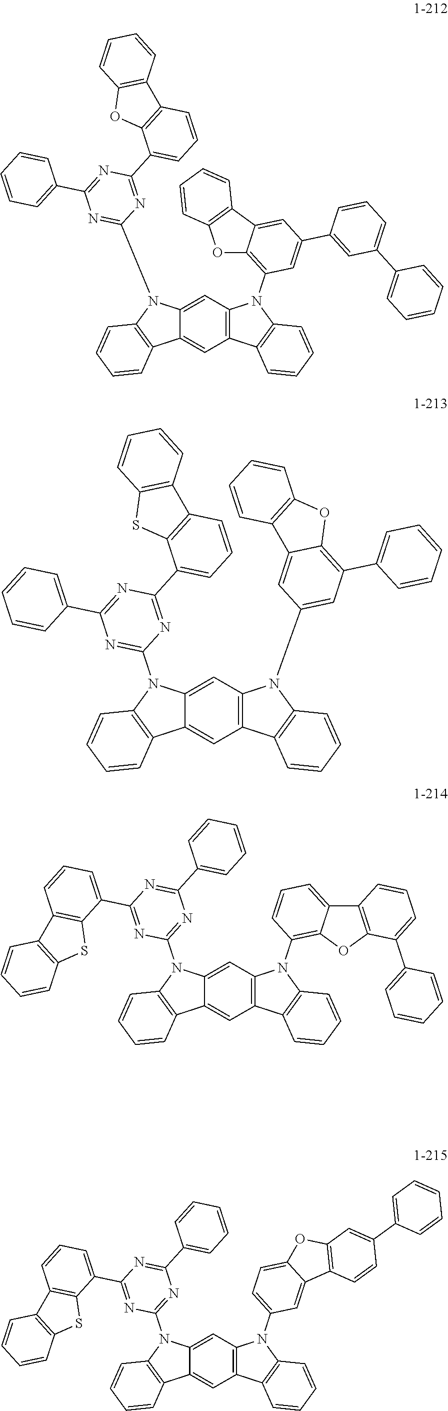 [SMILES: c1ccc(-c2cccc(-c3cc(-n4c5ccccc5c5cc6c7ccccc7n(-c7nc(-c8ccccc8)nc(-c8cccc9c8oc8ccccc89)n7)c6cc54)c4oc5ccccc5c4c3)c2)cc1 ; c1ccc(-c2nc(-c3cccc4c3sc3ccccc34)nc(-n3c4ccccc4c4cc5c6ccccc6n(-c6cc(-c7ccccc7)c7oc8ccccc8c7c6)c5cc43)n2)cc1 ; c1ccc(-c2nc(-c3cccc4c3sc3ccccc34)nc(-n3c4ccccc4c4cc5c6ccccc6n(-c6cccc7c6oc6c(-c8ccccc8)cccc67)c5cc43)n2)cc1 ; c1ccc(-c2ccc3c(c2)oc2ccc(-n4c5ccccc5c5cc6c7ccccc7n(-c7nc(-c8ccccc8)nc(-c8cccc9c8sc8ccccc89)n7)c6cc54)cc23)cc1]

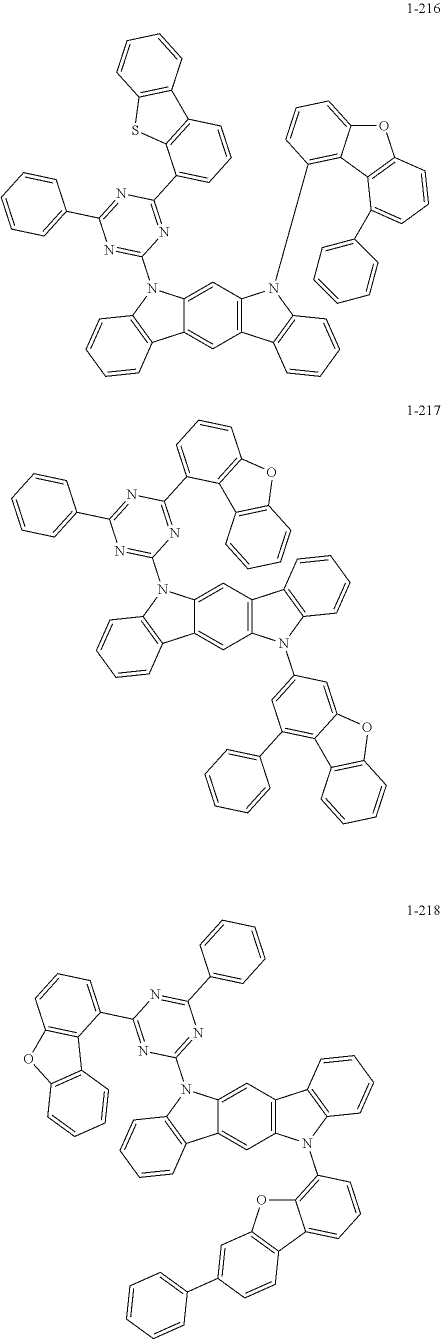 [SMILES: c1ccc(-c2nc(-c3cccc4c3sc3ccccc34)nc(-n3c4ccccc4c4cc5c6ccccc6n(-c6cccc7oc8cccc(-c9ccccc9)c8c67)c5cc43)n2)cc1 ; c1ccc(-c2nc(-c3cccc4oc5ccccc5c34)nc(-n3c4ccccc4c4cc5c(cc43)c3ccccc3n5-c3cc(-c4ccccc4)c4c(c3)oc3ccccc34)n2)cc1 ; c1ccc(-c2ccc3c(c2)oc2c(-n4c5ccccc5c5cc6c(cc54)c4ccccc4n6-c4nc(-c5ccccc5)nc(-c5cccc6oc7ccccc7c56)n4)cccc23)cc1]

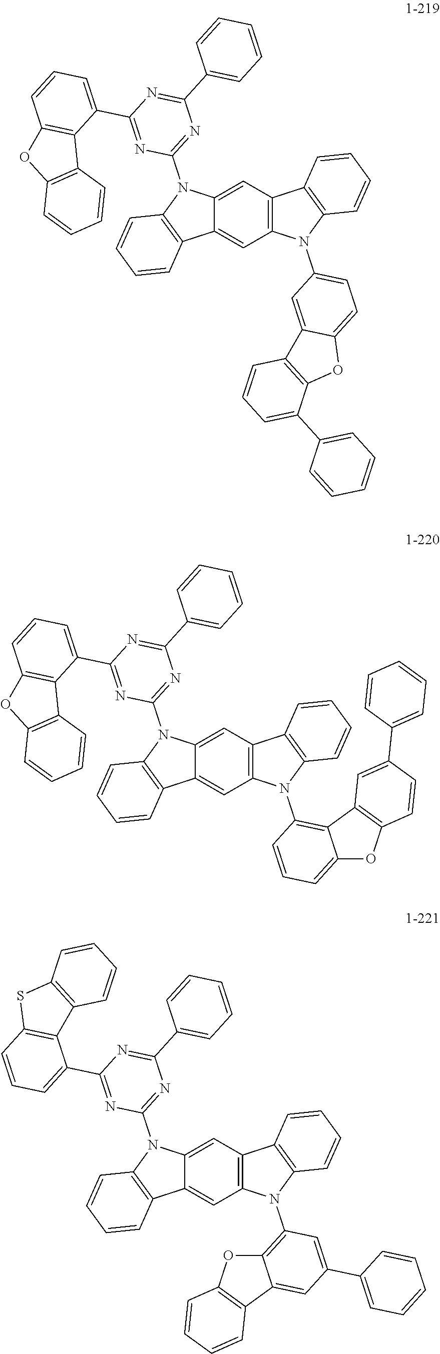 [SMILES: c1ccc(-c2nc(-c3cccc4oc5ccccc5c34)nc(-n3c4ccccc4c4cc5c(cc43)c3ccccc3n5-c3ccc4oc5c(-c6ccccc6)cccc5c4c3)n2)cc1 ; c1ccc(-c2ccc3oc4cccc(-n5c6ccccc6c6cc7c(cc65)c5ccccc5n7-c5nc(-c6ccccc6)nc(-c6cccc7oc8ccccc8c67)n5)c4c3c2)cc1 ; c1ccc(-c2cc(-n3c4ccccc4c4cc5c(cc43)c3ccccc3n5-c3nc(-c4ccccc4)nc(-c4cccc5sc6ccccc6c45)n3)c3oc4ccccc4c3c2)cc1]

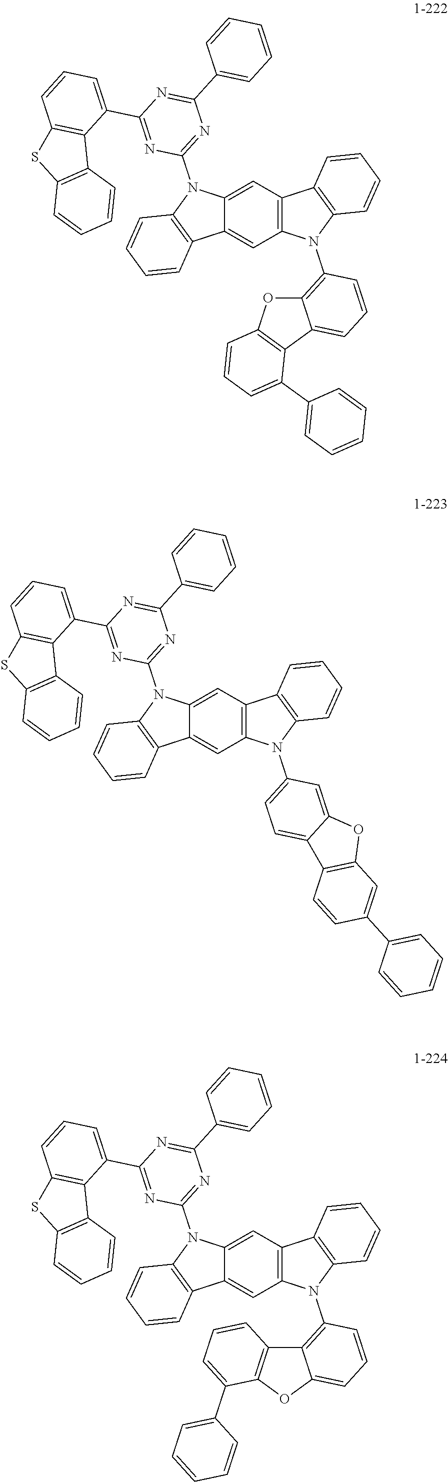 [SMILES: c1ccc(-c2nc(-c3cccc4sc5ccccc5c34)nc(-n3c4ccccc4c4cc5c(cc43)c3ccccc3n5-c3cccc4c3oc3cccc(-c5ccccc5)c34)n2)cc1 ; c1ccc(-c2ccc3c(c2)oc2cc(-n4c5ccccc5c5cc6c(cc54)c4ccccc4n6-c4nc(-c5ccccc5)nc(-c5cccc6sc7ccccc7c56)n4)ccc23)cc1 ; c1ccc(-c2nc(-c3cccc4sc5ccccc5c34)nc(-n3c4ccccc4c4cc5c(cc43)c3ccccc3n5-c3cccc4oc5c(-c6ccccc6)cccc5c34)n2)cc1]

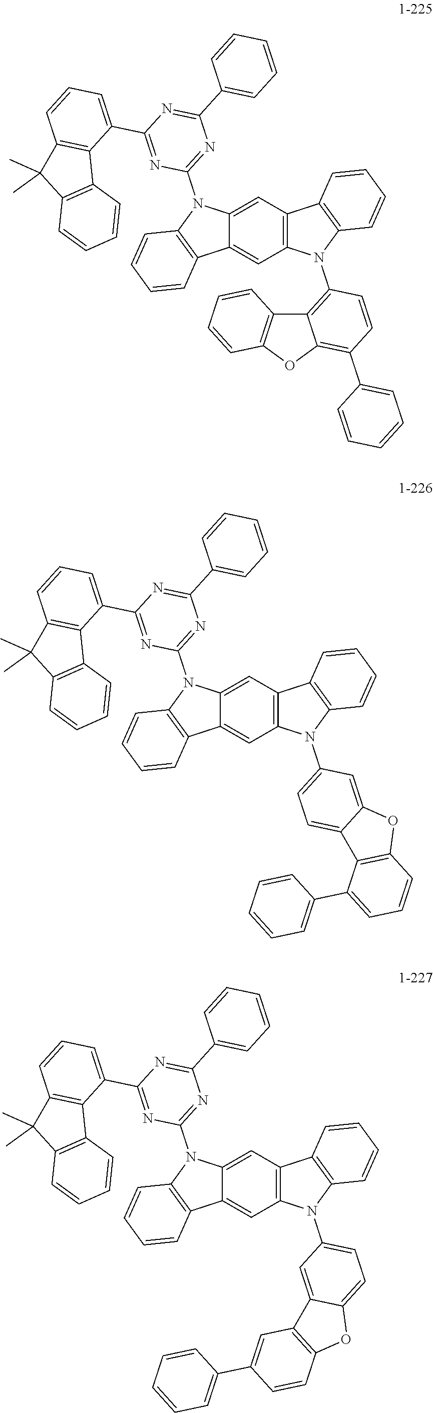 [SMILES: CC1(C)c2ccccc2-c2c(-c3nc(-c4ccccc4)nc(-n4c5ccccc5c5cc6c(cc54)c4ccccc4n6-c4ccc(-c5ccccc5)c5oc6ccccc6c45)n3)cccc21 ; CC1(C)c2ccccc2-c2c(-c3nc(-c4ccccc4)nc(-n4c5ccccc5c5cc6c(cc54)c4ccccc4n6-c4ccc5c(c4)oc4cccc(-c6ccccc6)c45)n3)cccc21 ; CC1(C)c2ccccc2-c2c(-c3nc(-c4ccccc4)nc(-n4c5ccccc5c5cc6c(cc54)c4ccccc4n6-c4ccc5oc6ccc(-c7ccccc7)cc6c5c4)n3)cccc21]

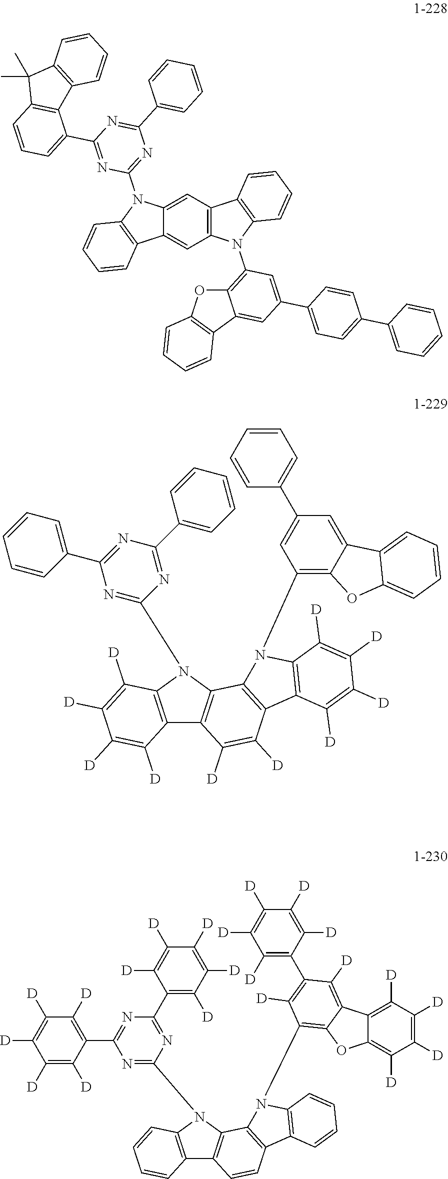 [SMILES: CC1(C)c2ccccc2-c2c(-c3nc(-c4ccccc4)nc(-n4c5ccccc5c5cc6c(cc54)c4ccccc4n6-c4cc(-c5ccc(-c6ccccc6)cc5)cc5c4oc4ccccc45)n3)cccc21 ; [2H]c1c([2H])c([2H])c2c(c1[2H])c1c([2H])c([2H])c3c4c([2H])c([2H])c([2H])c([2H])c4n(-c4cc(-c5ccccc5)cc5c4oc4ccccc45)c3c1n2-c1nc(-c2ccccc2)nc(-c2ccccc2)n1 ; [2H]c1c([2H])c([2H])c(-c2nc(-c3c([2H])c([2H])c([2H])c([2H])c3[2H])nc(-n3c4ccccc4c4ccc5c6ccccc6n(-c6c([2H])c(-c7c([2H])c([2H])c([2H])c([2H])c7[2H])c([2H])c7c6oc6c([2H])c([2H])c([2H])c([2H])c67)c5c43)n2)c([2H])c1[2H]]

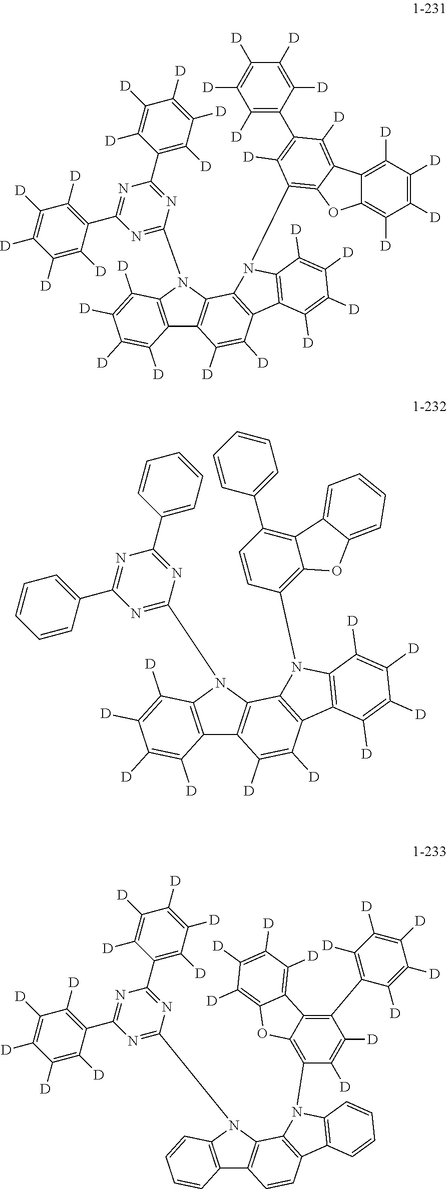

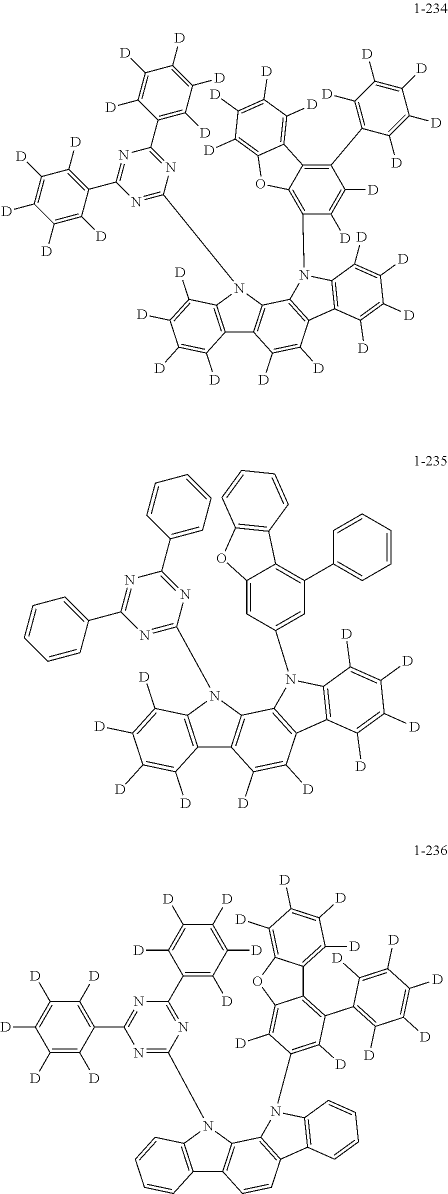

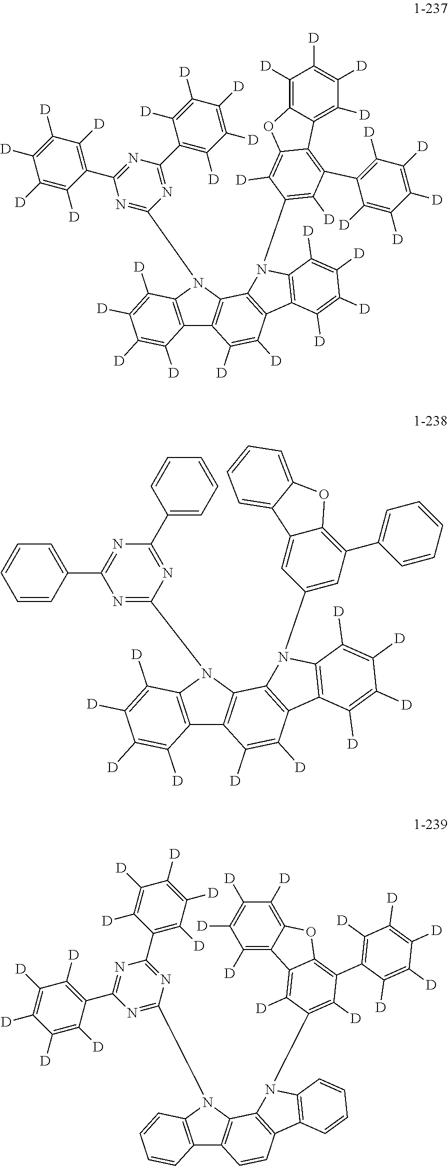

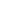

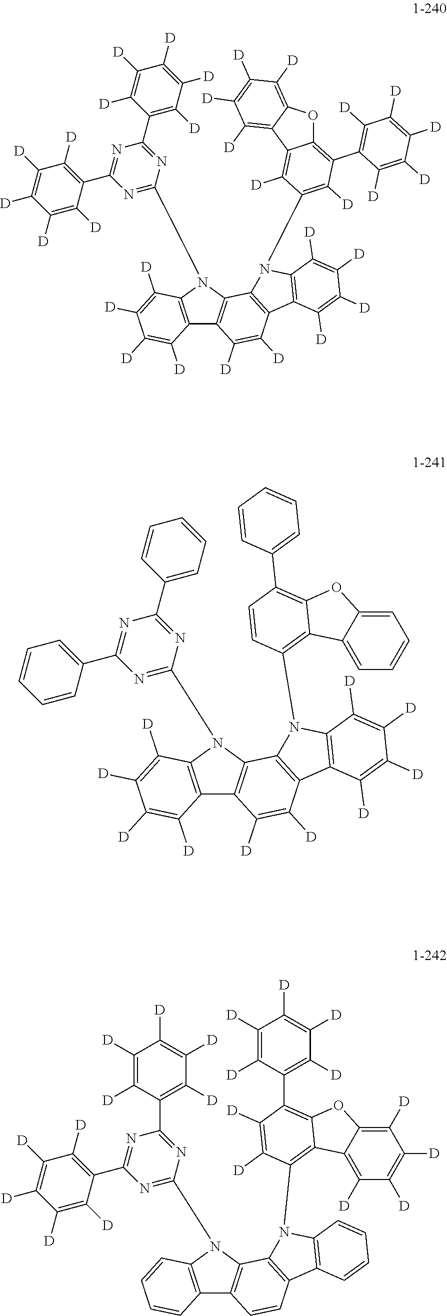

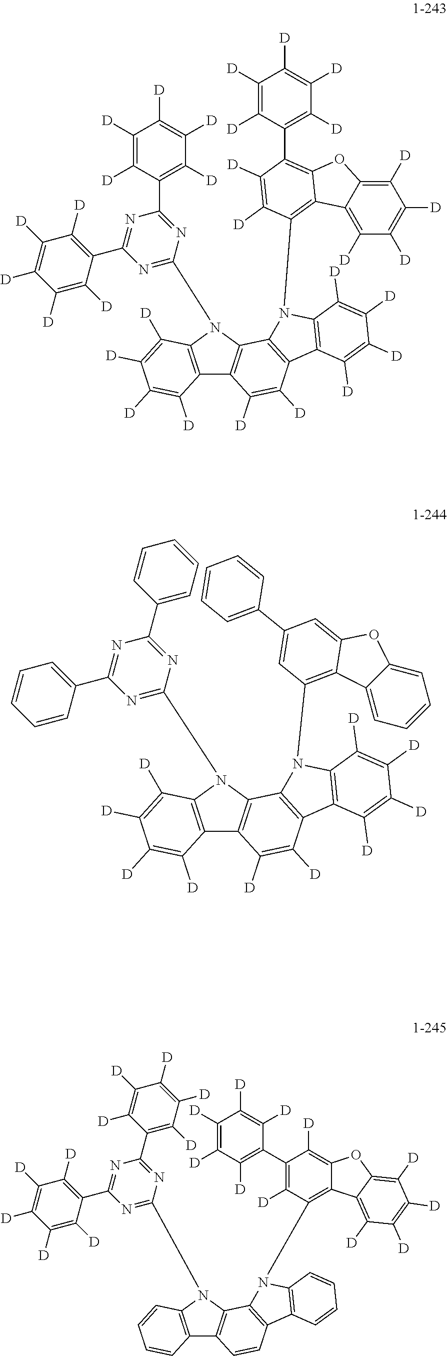

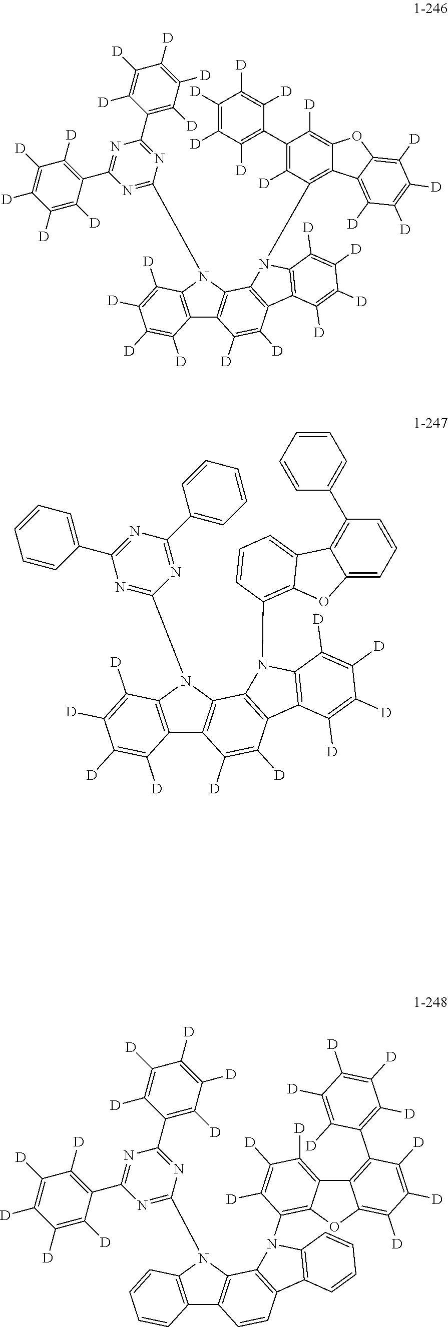

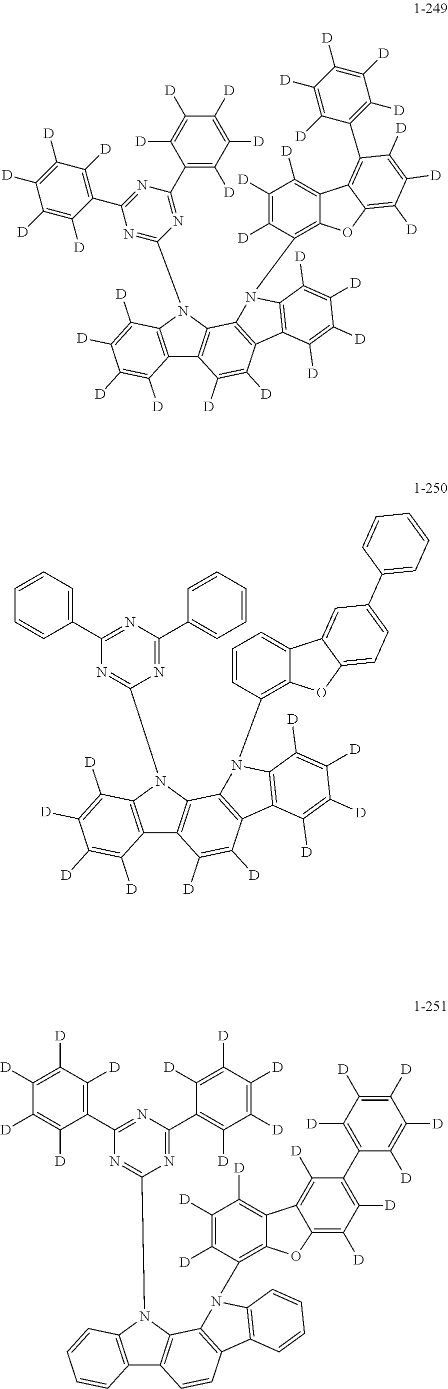

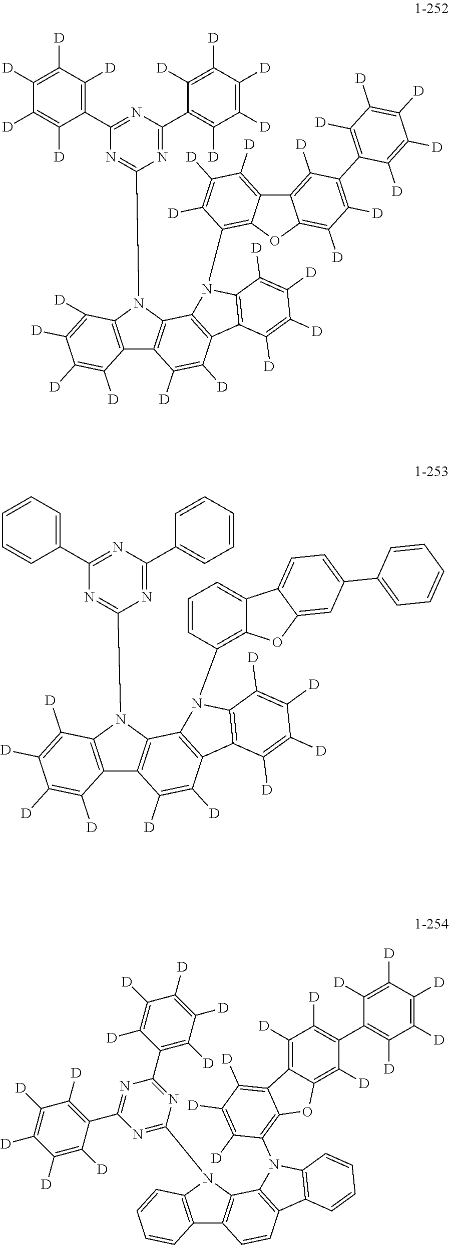

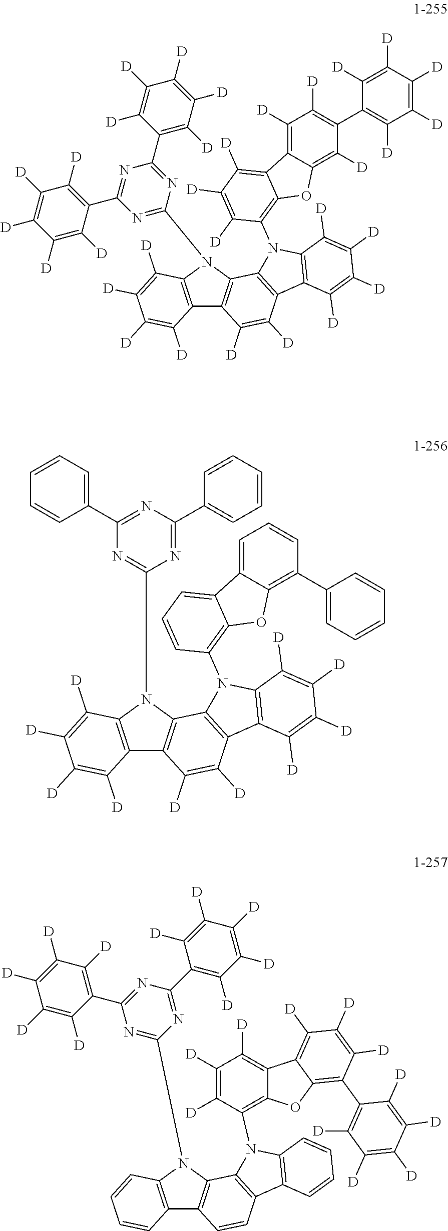

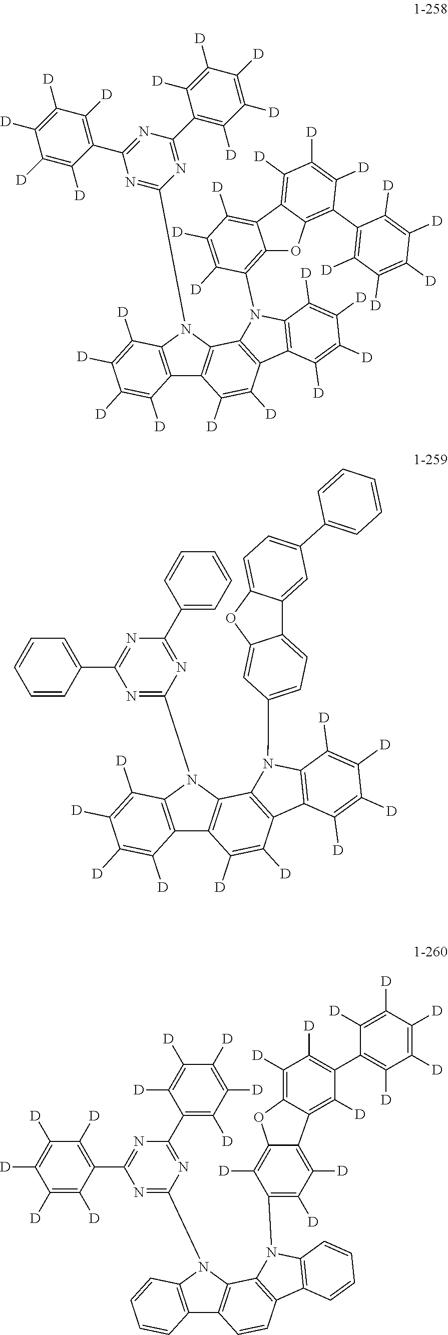

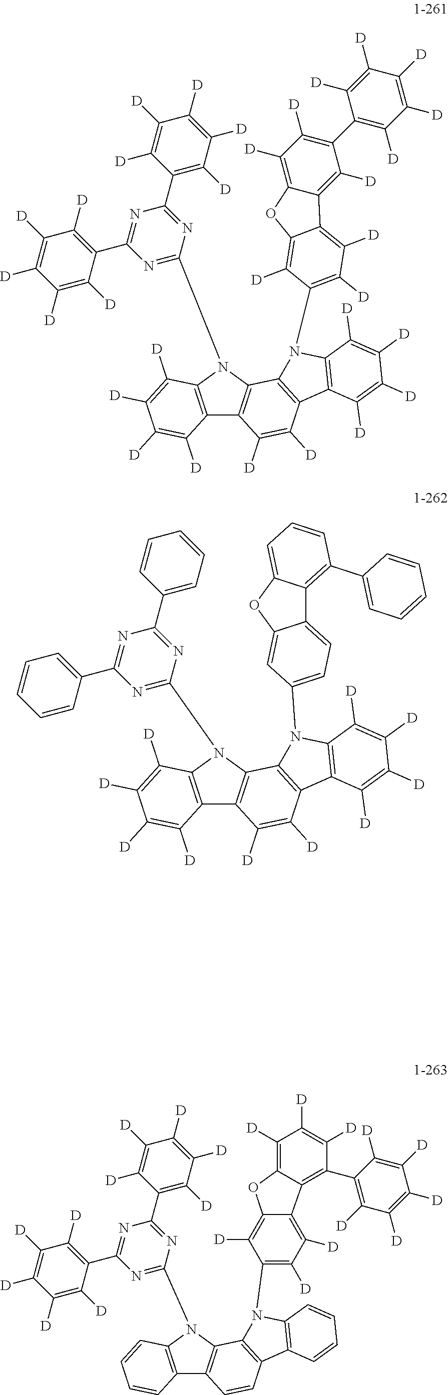

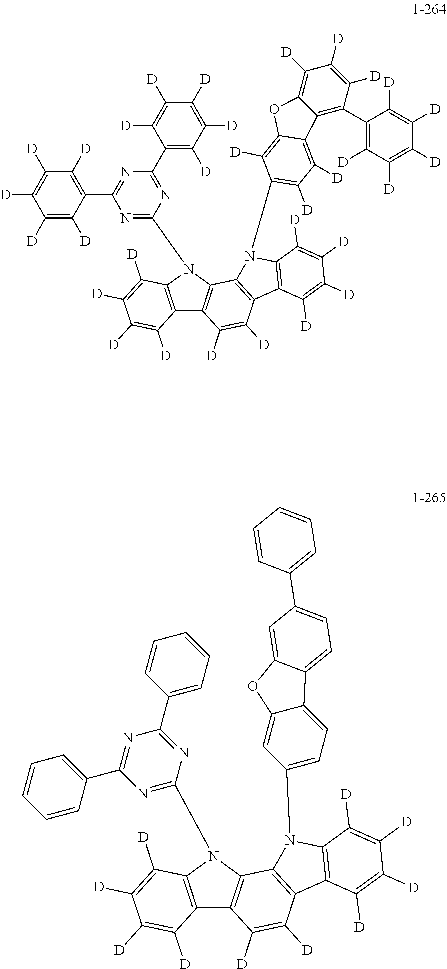 [SMILES: [2H]c1c([2H])c([2H])c(-c2nc(-c3c([2H])c([2H])c([2H])c([2H])c3[2H])nc(-n3c4c([2H])c([2H])c([2H])c([2H])c4c4c([2H])c([2H])c5c6c([2H])c([2H])c([2H])c([2H])c6n(-c6c([2H])c([2H])c7c(oc8c([2H])c([2H])c([2H])c(-c9c([2H])c([2H])c([2H])c([2H])c9[2H])c87)c6[2H])c5c43)n2)c([2H])c1[2H] ; [2H]c1c([2H])c([2H])c2c(c1[2H])c1c([2H])c([2H])c3c4c([2H])c([2H])c([2H])c([2H])c4n(-c4nc(-c5ccccc5)nc(-c5ccccc5)n4)c3c1n2-c1ccc2c(c1)oc1cc(-c3ccccc3)ccc12]

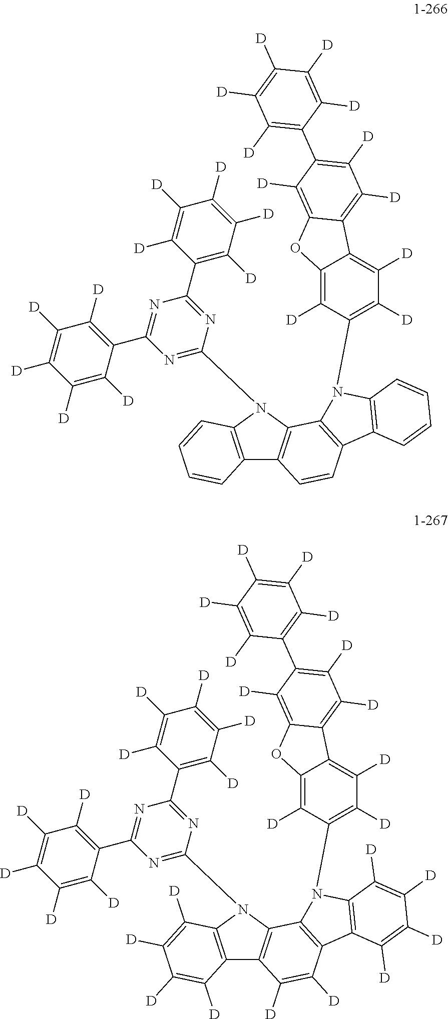 [SMILES: [2H]c1c([2H])c([2H])c(-c2nc(-c3c([2H])c([2H])c([2H])c([2H])c3[2H])nc(-n3c4ccccc4c4ccc5c6ccccc6n(-c6c([2H])c([2H])c7c(oc8c([2H])c(-c9c([2H])c([2H])c([2H])c([2H])c9[2H])c([2H])c([2H])c87)c6[2H])c5c43)n2)c([2H])c1[2H] ; [2H]c1c([2H])c([2H])c(-c2nc(-c3c([2H])c([2H])c([2H])c([2H])c3[2H])nc(-n3c4c([2H])c([2H])c([2H])c([2H])c4c4c([2H])c([2H])c5c6c([2H])c([2H])c([2H])c([2H])c6n(-c6c([2H])c([2H])c7c(oc8c([2H])c(-c9c([2H])c([2H])c([2H])c([2H])c9[2H])c([2H])c([2H])c87)c6[2H])c5c43)n2)c([2H])c1[2H]]

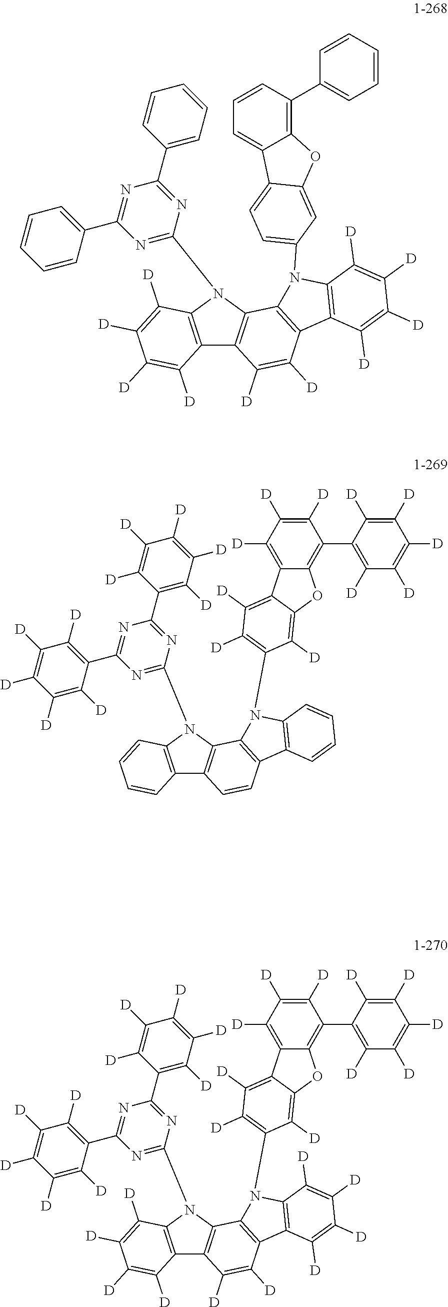

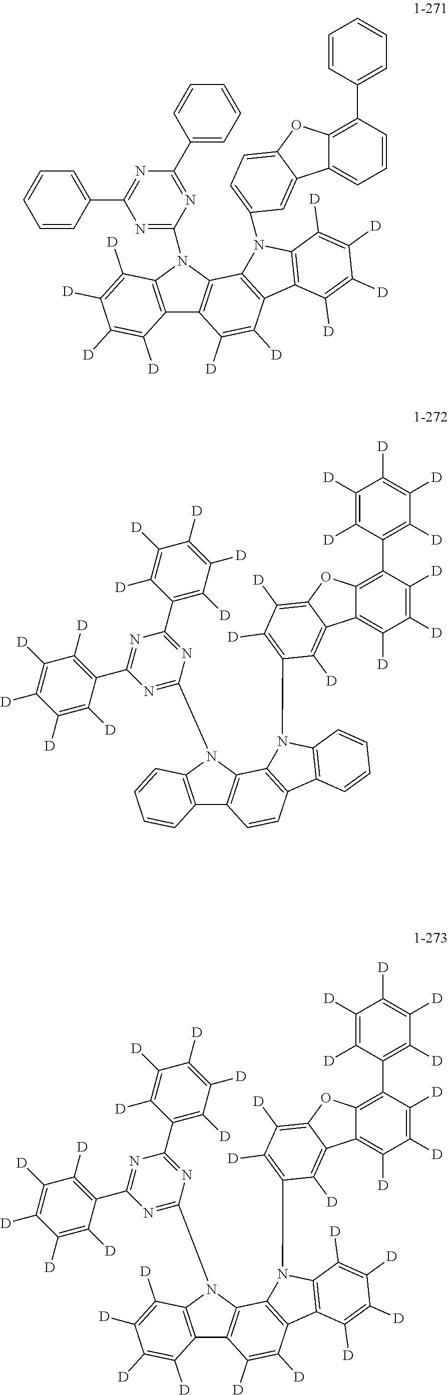

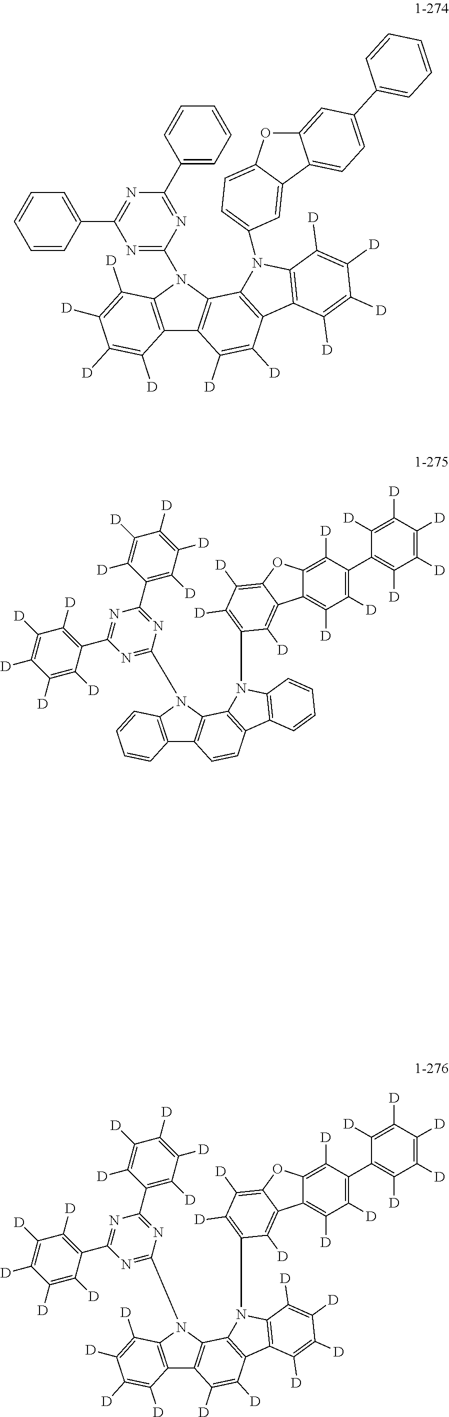

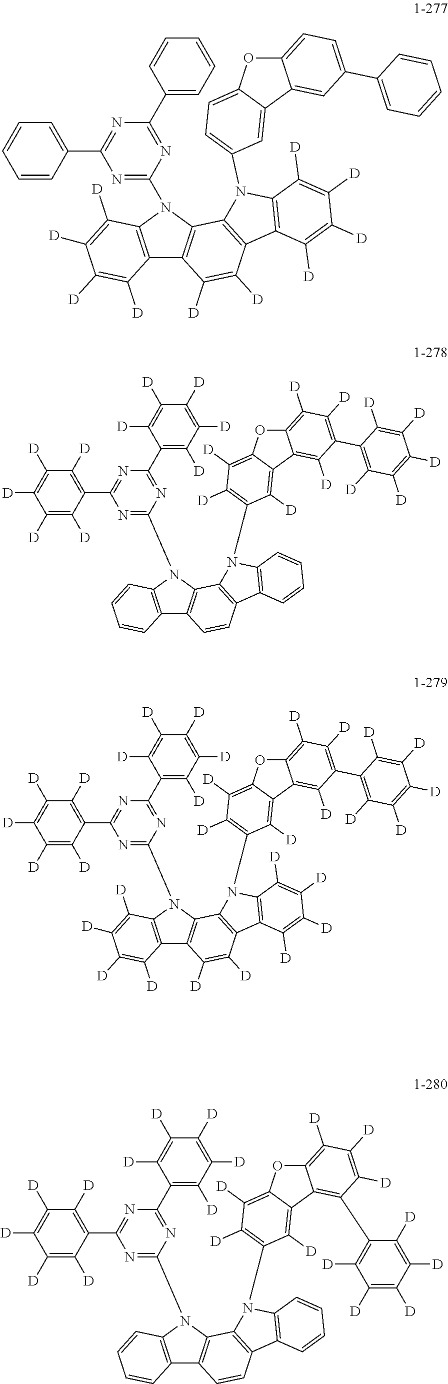

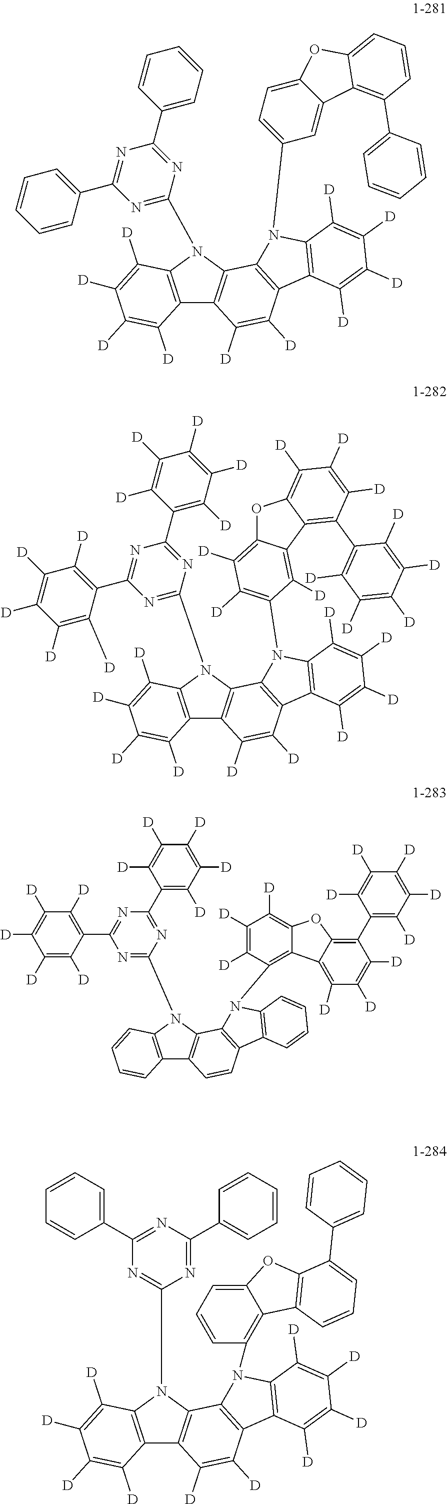

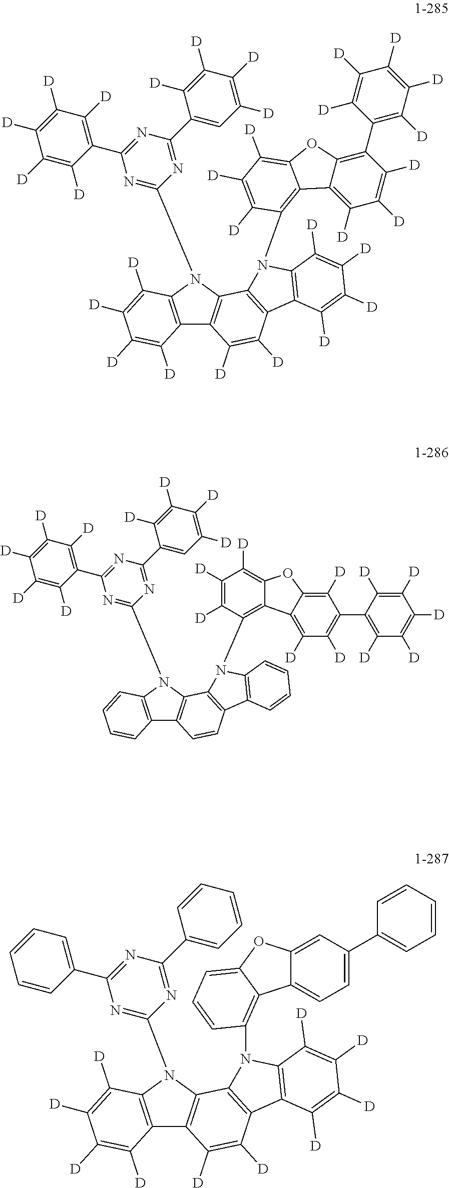

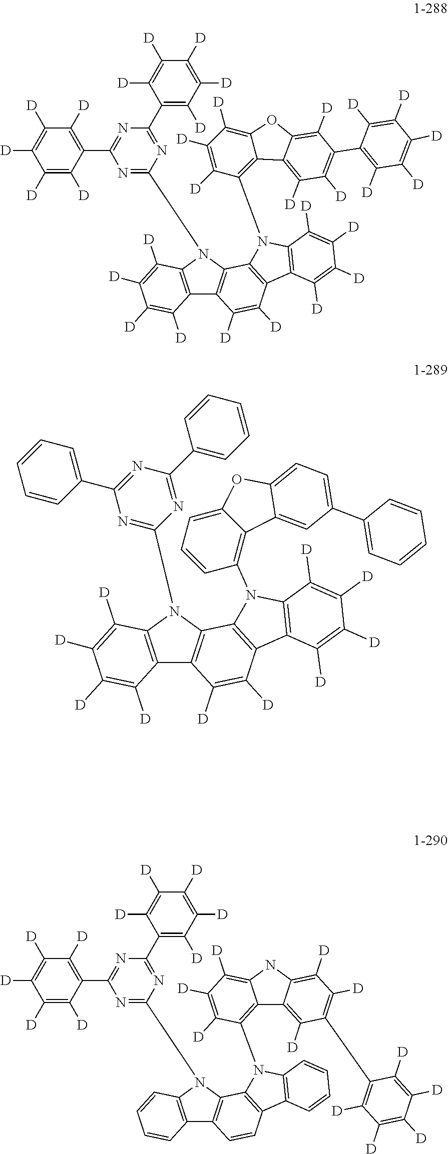

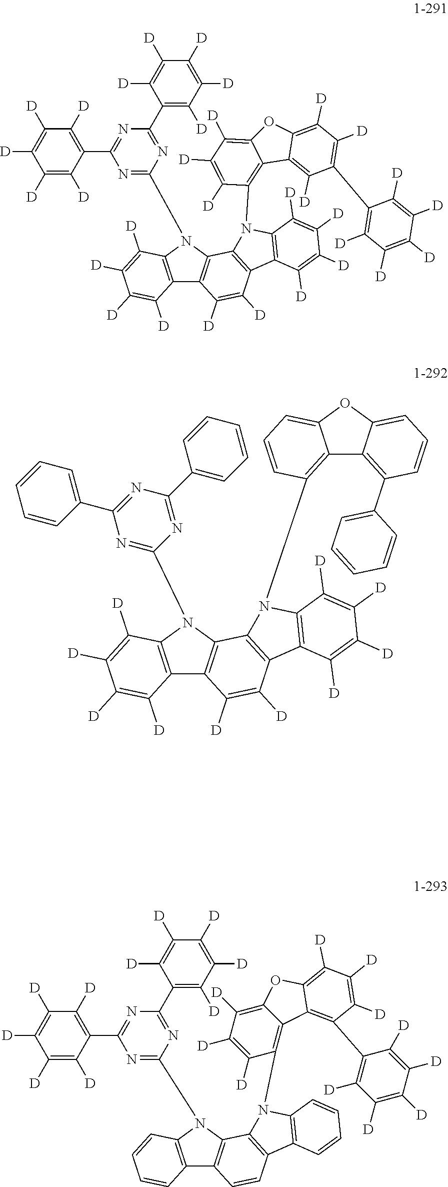

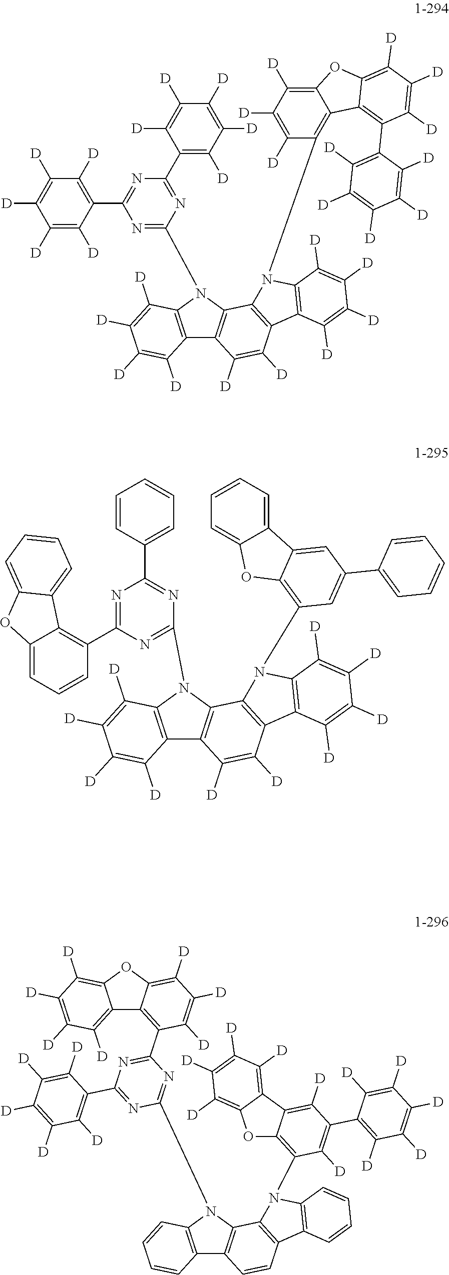

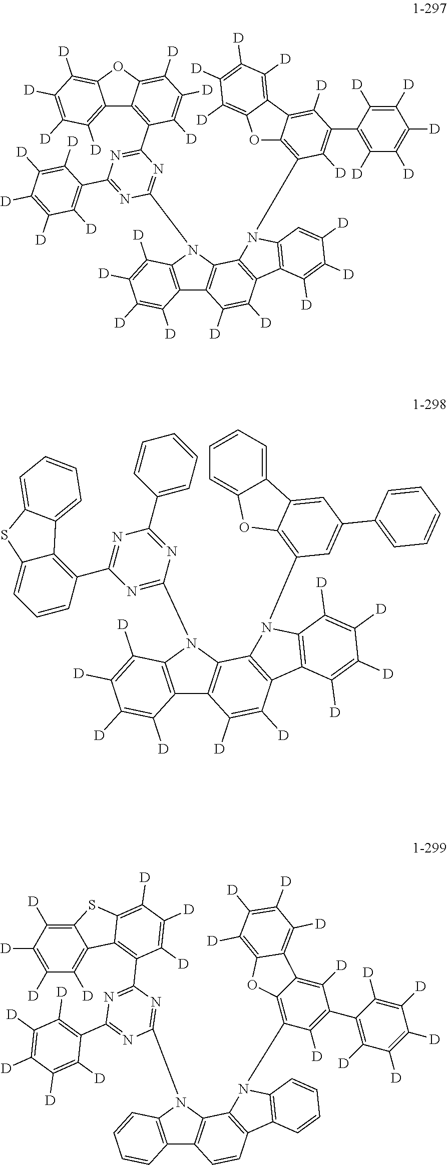

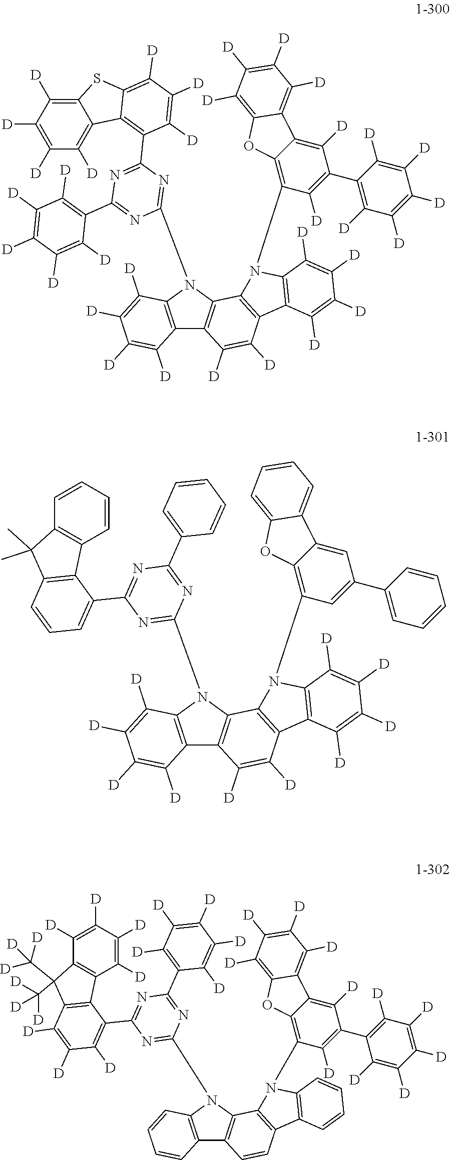

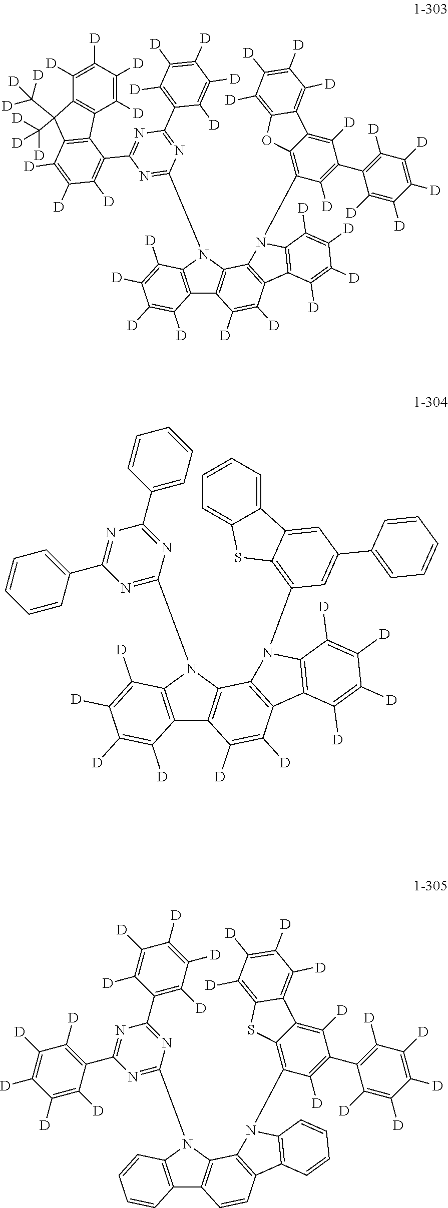

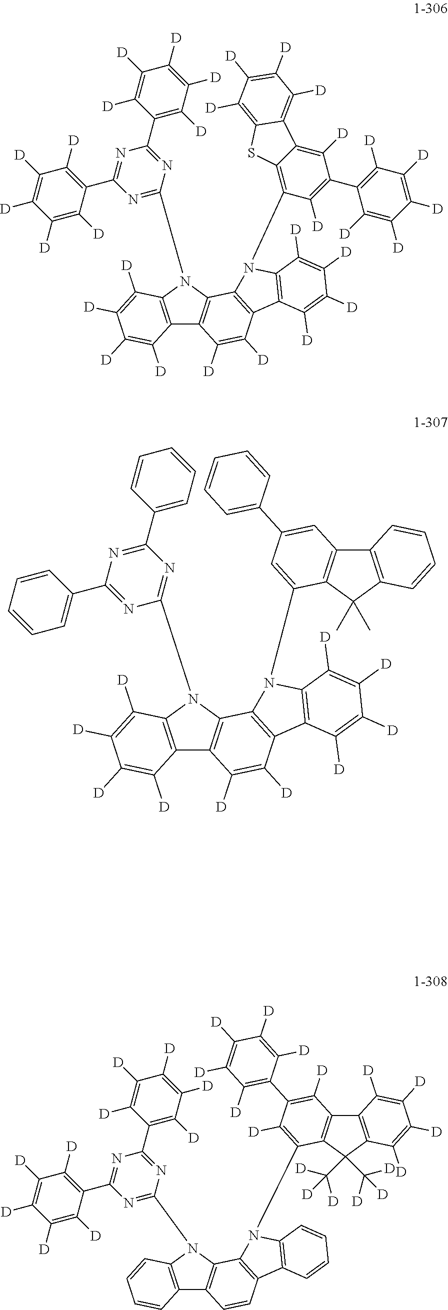

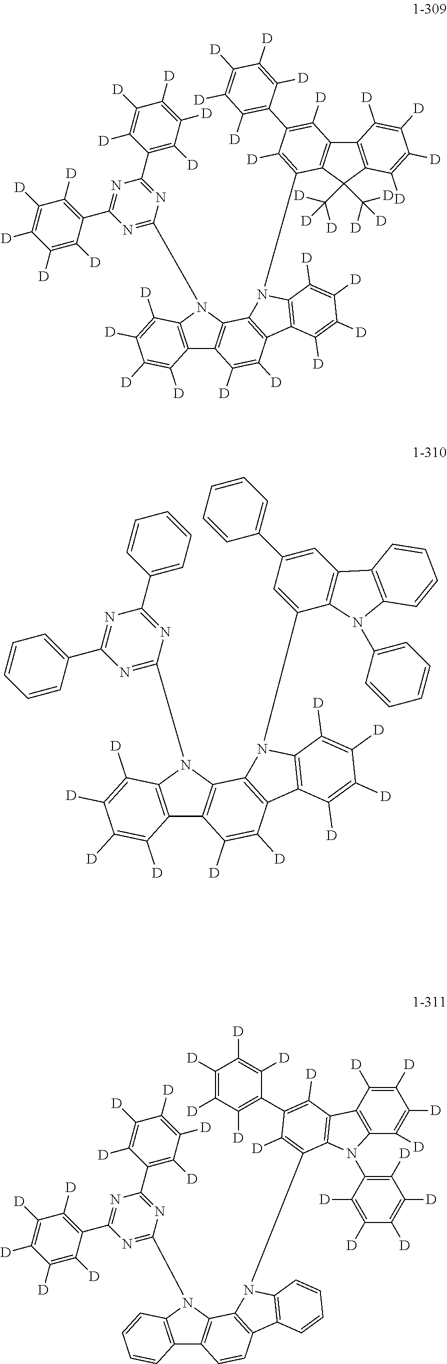

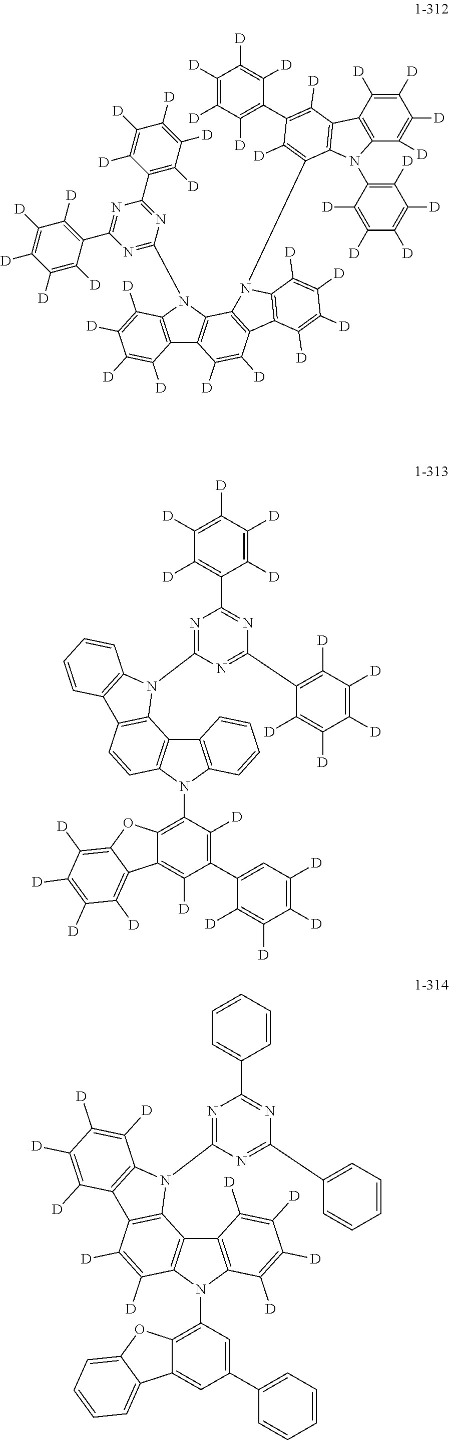

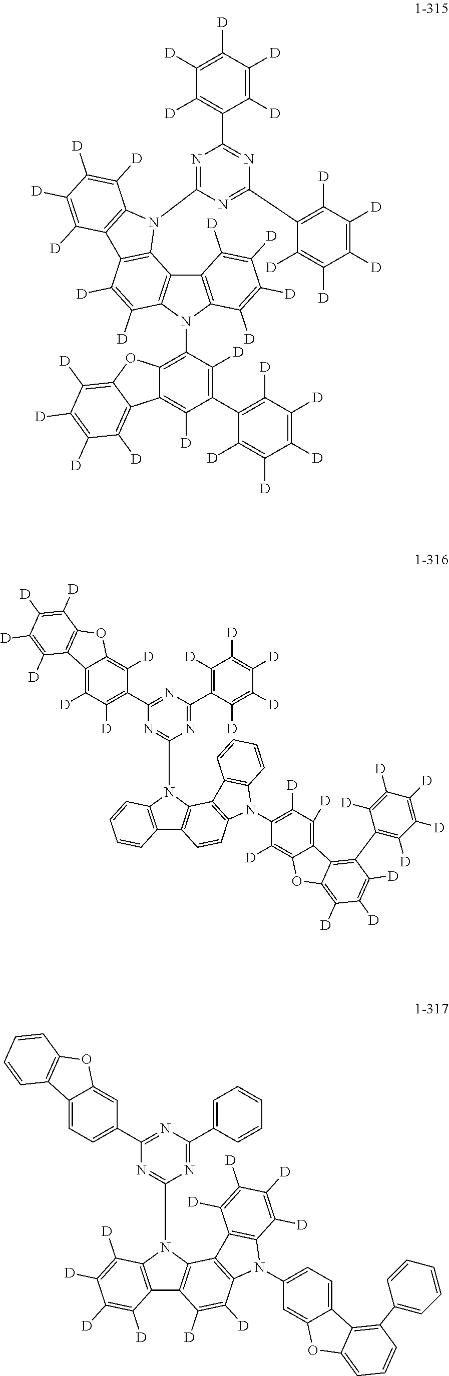

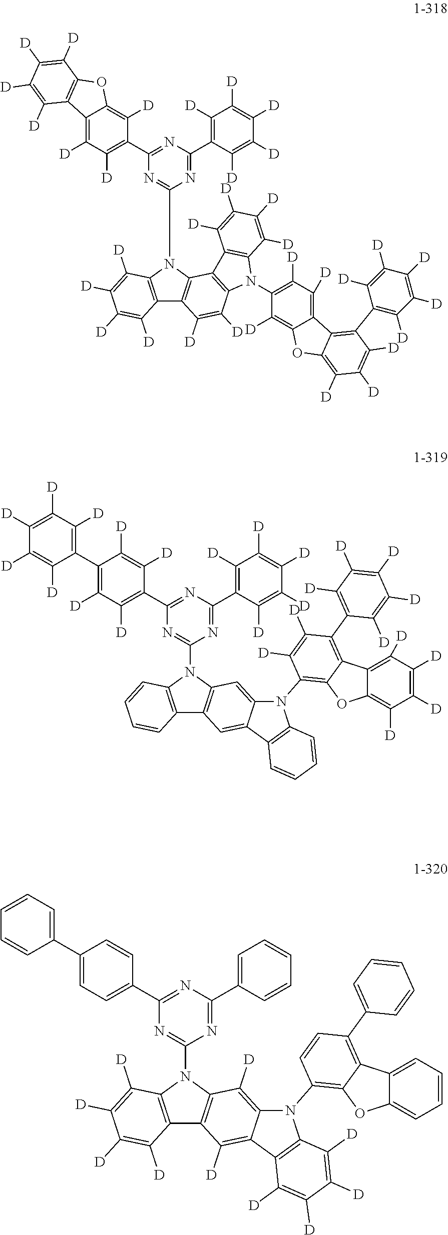

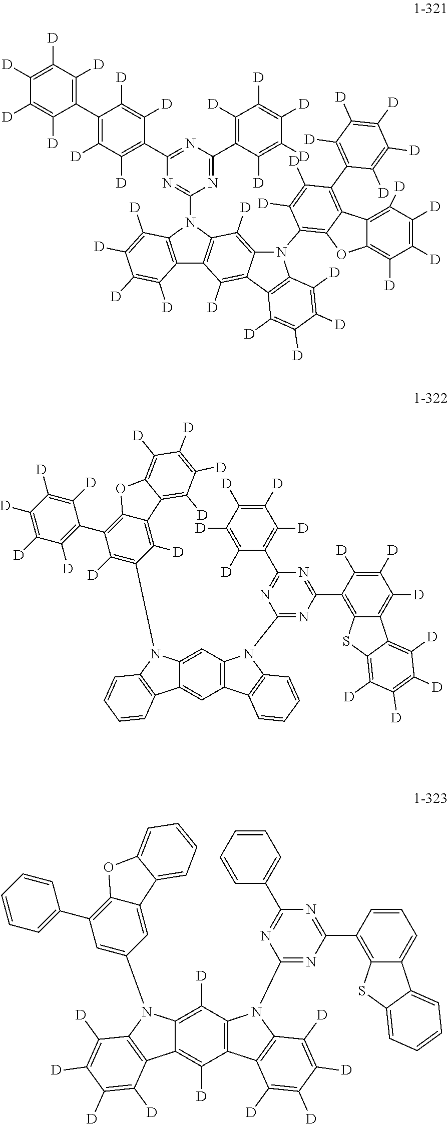

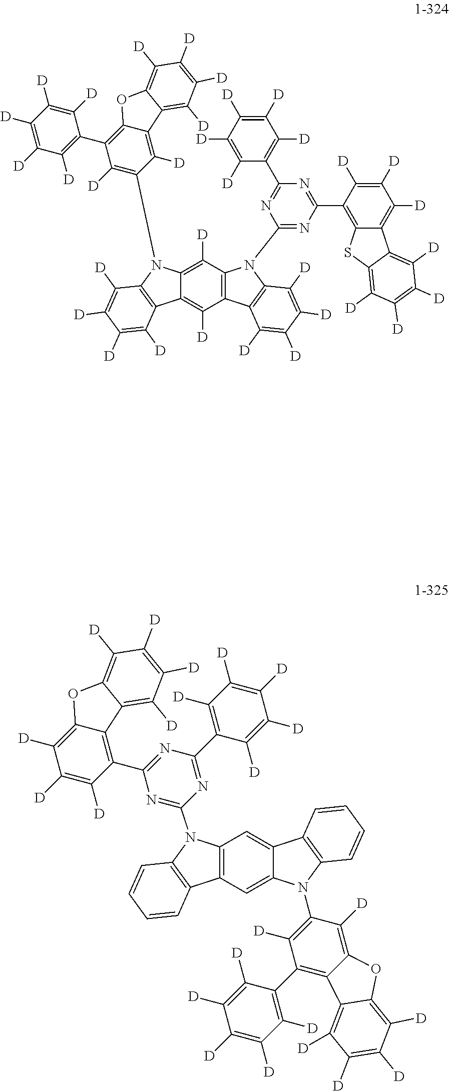

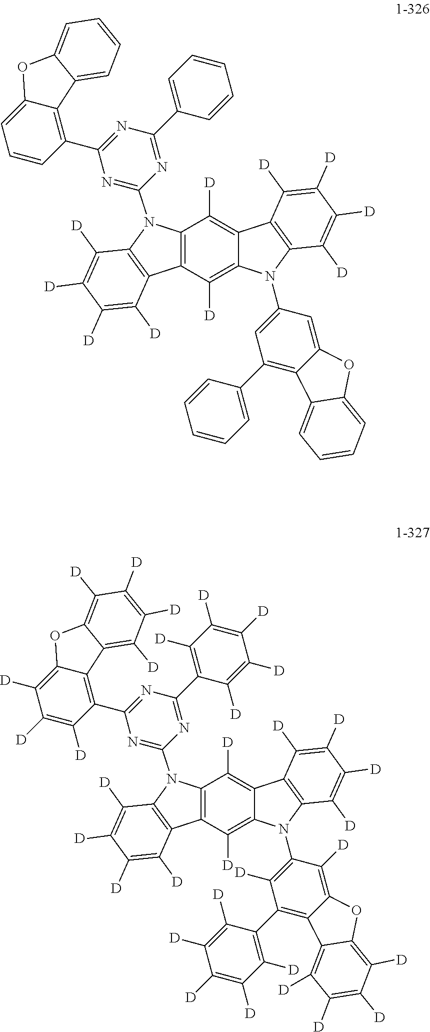 [SMILES: [2H]c1c([2H])c([2H])c2c(c1[2H])c1c([2H])c3c(c([2H])c1n2-c1cc(-c2ccccc2)c2c(c1)oc1ccccc12)c1c([2H])c([2H])c([2H])c([2H])c1n3-c1nc(-c2ccccc2)nc(-c2cccc3oc4ccccc4c23)n1 ; [2H]c1c([2H])c([2H])c(-c2nc(-c3c([2H])c([2H])c([2H])c4oc5c([2H])c([2H])c([2H])c([2H])c5c34)nc(-n3c4c([2H])c([2H])c([2H])c([2H])c4c4c([2H])c5c(c([2H])c43)c3c([2H])c([2H])c([2H])c([2H])c3n5-c3c([2H])c(-c4c([2H])c([2H])c([2H])c([2H])c4[2H])c4c(oc5c([2H])c([2H])c([2H])c([2H])c54)c3[2H])n2)c([2H])c1[2H]]

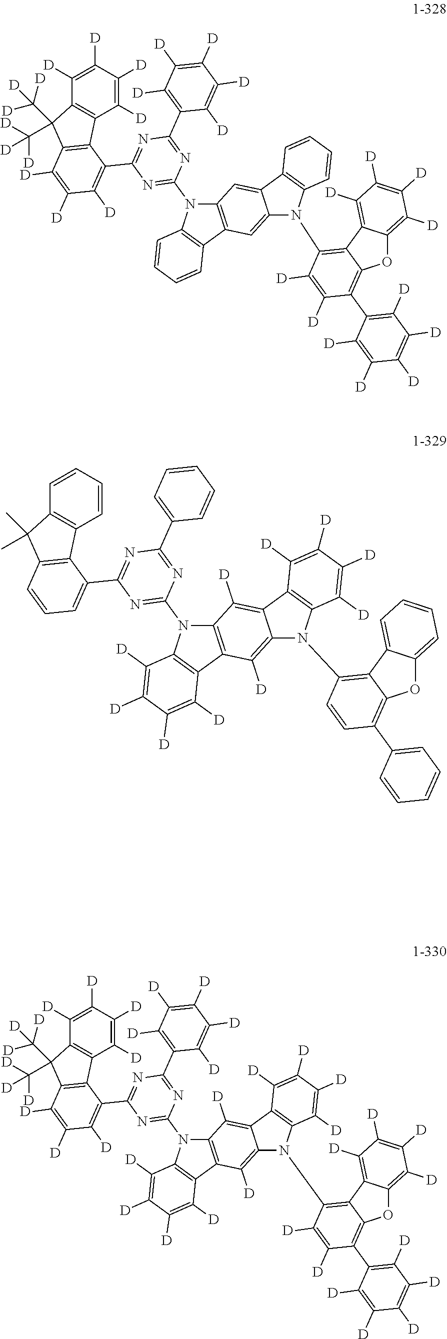

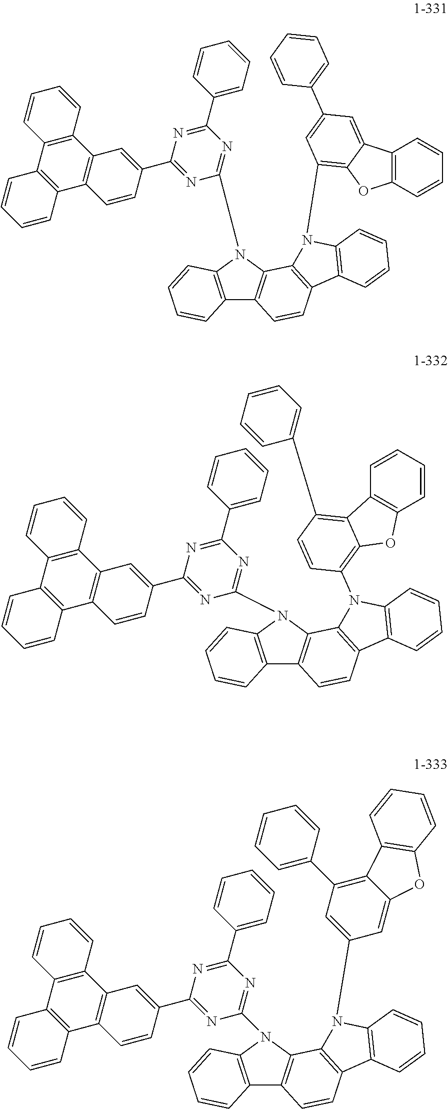 [SMILES: c1ccc(-c2cc(-n3c4ccccc4c4ccc5c6ccccc6n(-c6nc(-c7ccccc7)nc(-c7ccc8c9ccccc9c9ccccc9c8c7)n6)c5c43)c3oc4ccccc4c3c2)cc1 ; c1ccc(-c2nc(-c3ccc4c5ccccc5c5ccccc5c4c3)nc(-n3c4ccccc4c4ccc5c6ccccc6n(-c6ccc(-c7ccccc7)c7c6oc6ccccc67)c5c43)n2)cc1 ; c1ccc(-c2nc(-c3ccc4c5ccccc5c5ccccc5c4c3)nc(-n3c4ccccc4c4ccc5c6ccccc6n(-c6cc(-c7ccccc7)c7c(c6)oc6ccccc67)c5c43)n2)cc1]

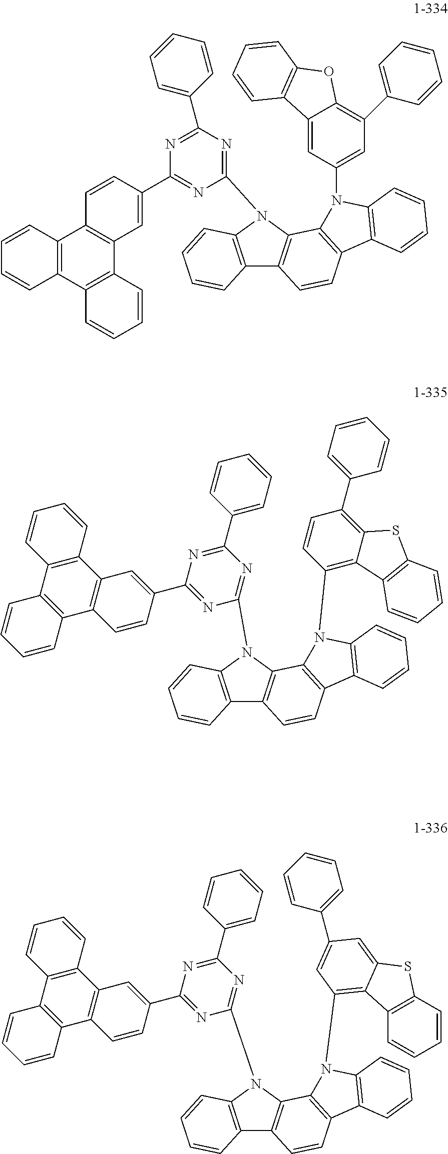 [SMILES: c1ccc(-c2nc(-c3ccc4c5ccccc5c5ccccc5c4c3)nc(-n3c4ccccc4c4ccc5c6ccccc6n(-c6cc(-c7ccccc7)c7oc8ccccc8c7c6)c5c43)n2)cc1 ; c1ccc(-c2nc(-c3ccc4c5ccccc5c5ccccc5c4c3)nc(-n3c4ccccc4c4ccc5c6ccccc6n(-c6ccc(-c7ccccc7)c7sc8ccccc8c67)c5c43)n2)cc1 ; c1ccc(-c2cc(-n3c4ccccc4c4ccc5c6ccccc6n(-c6nc(-c7ccccc7)nc(-c7ccc8c9ccccc9c9ccccc9c8c7)n6)c5c43)c3c(c2)sc2ccccc23)cc1]

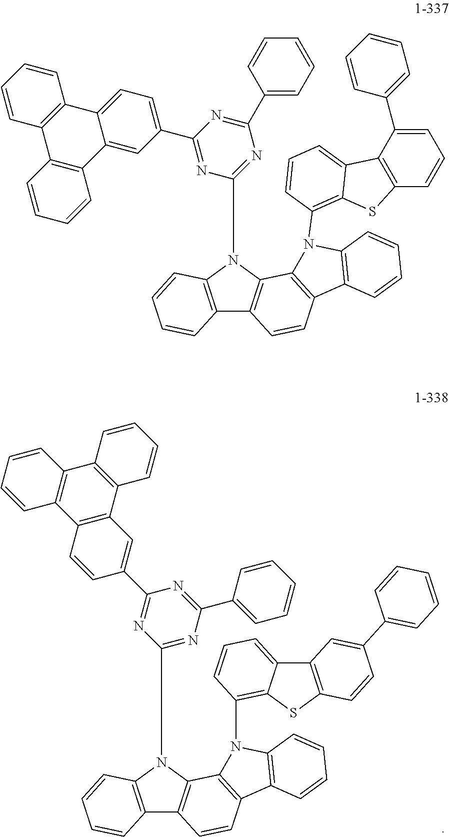 [SMILES: c1ccc(-c2nc(-c3ccc4c5ccccc5c5ccccc5c4c3)nc(-n3c4ccccc4c4ccc5c6ccccc6n(-c6cccc7c6sc6cccc(-c8ccccc8)c67)c5c43)n2)cc1 ; c1ccc(-c2ccc3sc4c(-n5c6ccccc6c6ccc7c8ccccc8n(-c8nc(-c9ccccc9)nc(-c9ccc%10c%11ccccc%11c%11ccccc%11c%10c9)n8)c7c65)cccc4c3c2)cc1]

7. An organic light emitting device comprising:
a first electrode;
a second electrode provided opposite to the first electrode; and
one or more organic material layers provided between the first electrode and the second electrode,
wherein one or more layers of the organic material layers comprise the heterocyclic compound of claim 1.

8. The organic light emitting device of claim 7, wherein the organic material layer comprises a light emitting layer, and the light emitting layer comprises the heterocyclic compound.

9. The organic light emitting device of claim 7, wherein the organic material layer comprises a light emitting layer, the light emitting layer comprises a host material, and the host material comprises the heterocyclic compound.

10. The organic light emitting device of claim 7, wherein the organic material layer further comprises a heterocyclic compound represented by the following Chemical Formula 10: 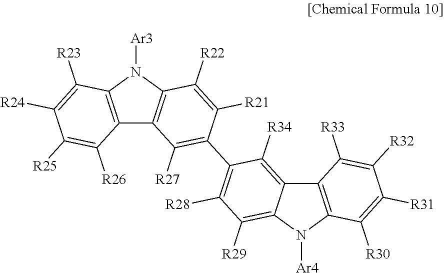
in Chemical Formula 10,
R21 to R34 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R301R302; —SiR301R302R303; and —NR301R302, or two or more groups adjacent to each other bond to each other to form a substituted or unsubstituted C6 to C60 aromatic hydrocarbon ring or a substituted or unsubstituted C2 to C60 heteroring, and R301, R302 and R303 are the same as or different from each other and each independently a substituted or unsubstituted C1 to C60 alkyl group; a substituted or unsubstituted C6 to C60 aryl group; or a substituted or unsubstituted C2 to C60 heteroaryl group; and
Ar3 and Ar4 are the same as or different from each other, and each independently a substituted or unsubstituted C6 to C60 aryl group; or a substituted or unsubstituted C2 to C60 heteroaryl group.

11. The organic light emitting device of claim 10, wherein the heterocyclic compound represented by Chemical Formula 10 does not include deuterium as a substituent, or a content of deuterium is from 1% to 100% based on a total number of hydrogen atoms and deuterium atoms.

12. The organic light emitting device of claim 10, wherein at least one of the heterocyclic compound represented by Chemical Formula 1 and the heterocyclic compound represented by Chemical Formula 10 does not include deuterium as a substituent, or a content of deuterium is from 1% to 100% based on a total number of hydrogen atoms and deuterium atoms.

13. The organic light emitting device of claim 10, wherein the heterocyclic compound represented by Chemical Formula 10 is any one selected from among the following compounds:

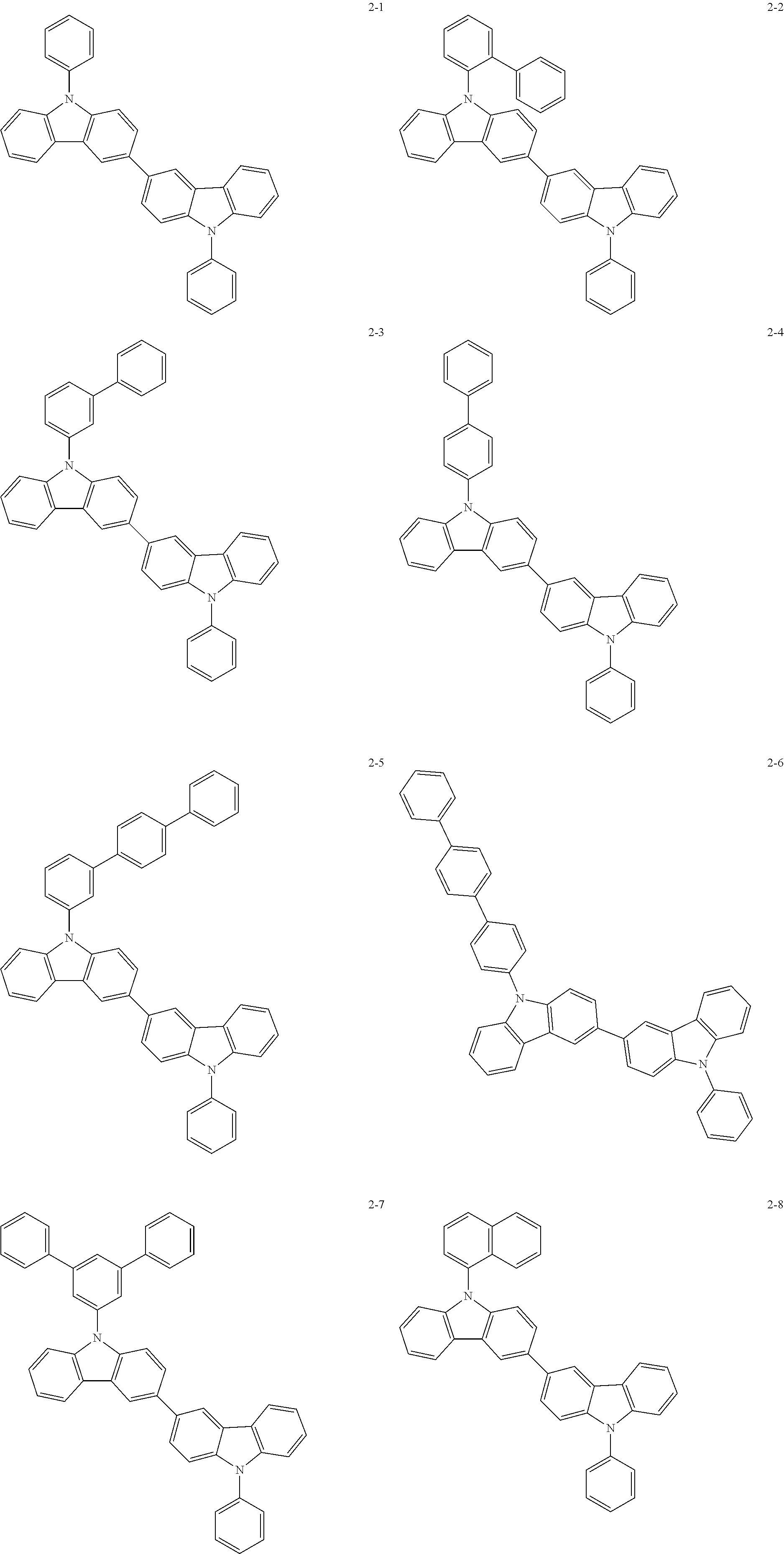

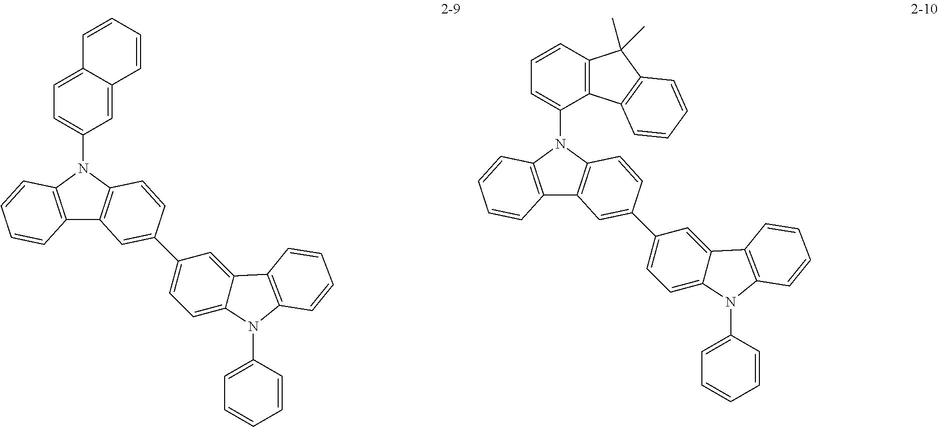 [SMILES: c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21]

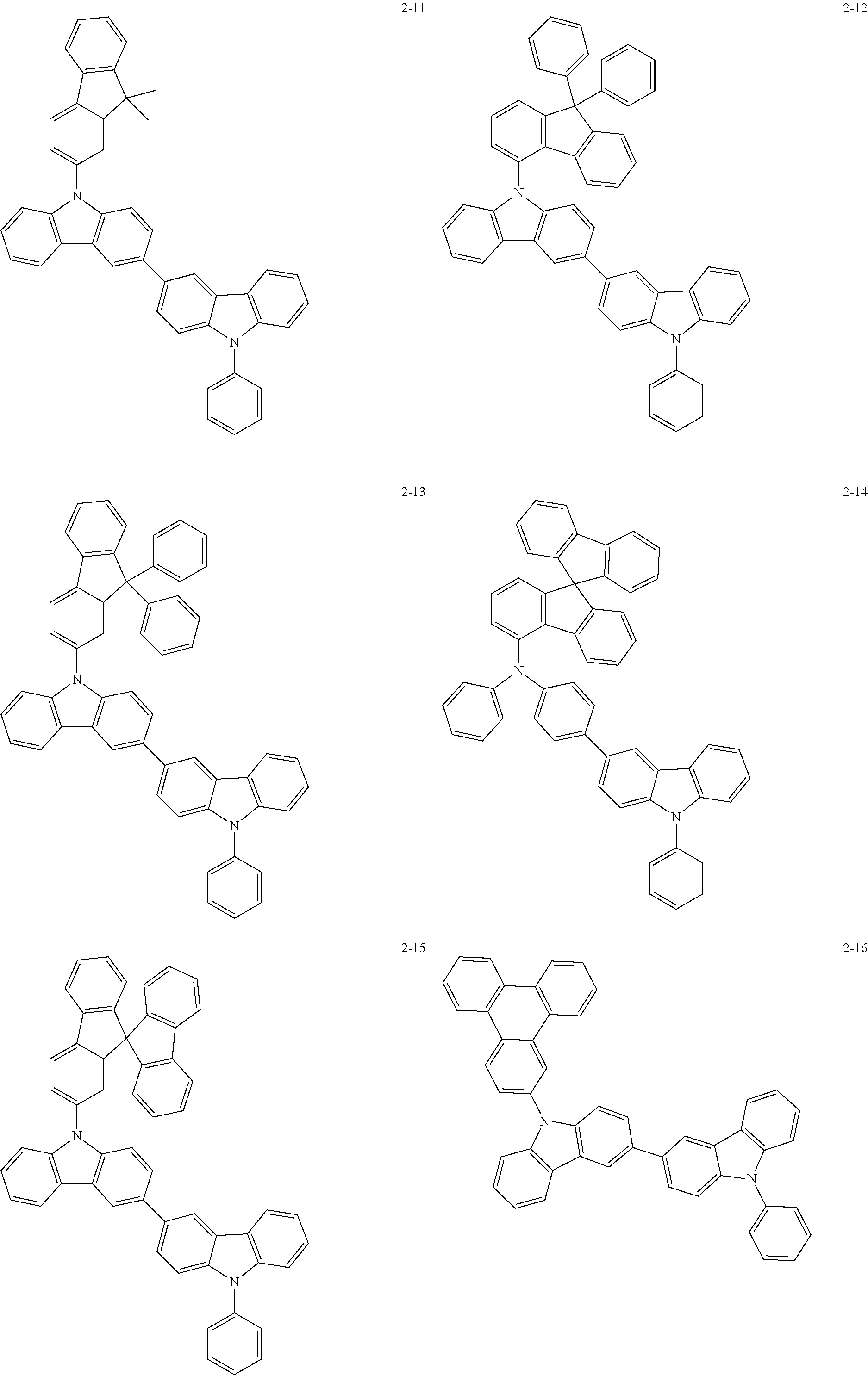

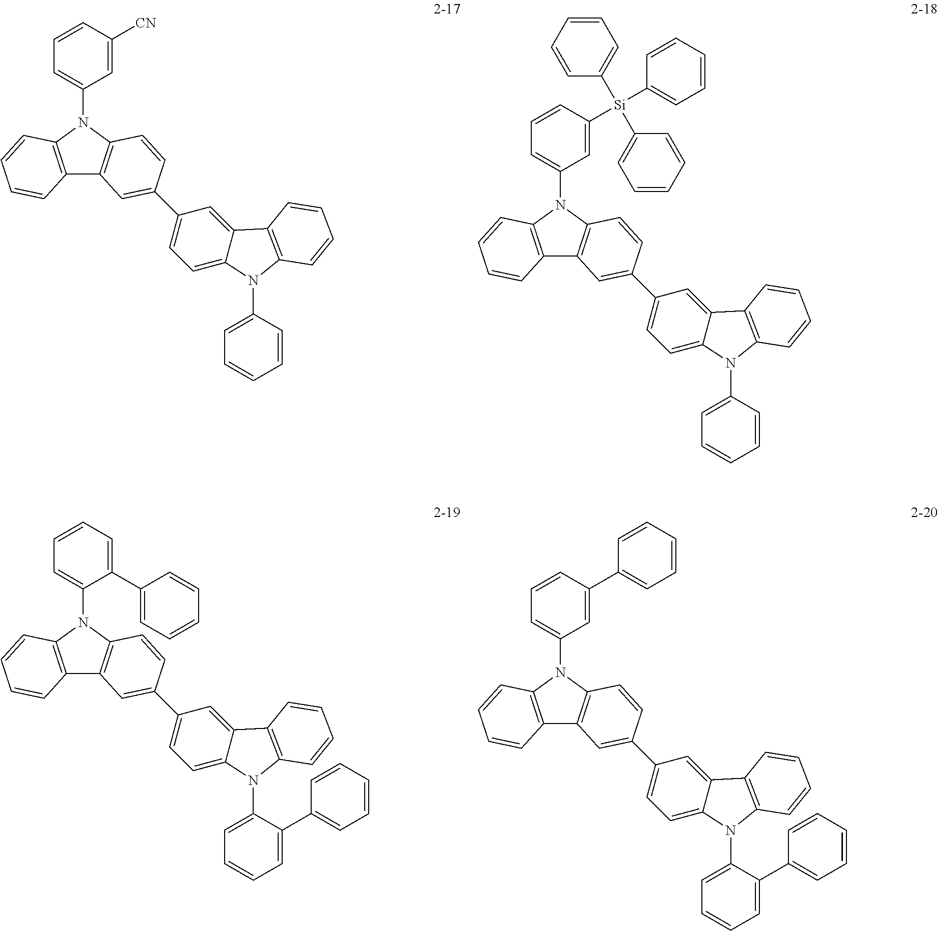 [SMILES: N#Cc1cccc(-n2c3ccccc3c3cc(-c4ccc5c(c4)c4ccccc4n5-c4ccccc4)ccc32)c1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1 ; c1ccc(-c2ccccc2-n2c3ccccc3c3cc(-c4ccc5c(c4)c4ccccc4n5-c4ccccc4-c4ccccc4)ccc32)cc1 ; c1ccc(-c2cccc(-n3c4ccccc4c4cc(-c5ccc6c(c5)c5ccccc5n6-c5ccccc5-c5ccccc5)ccc43)c2)cc1]

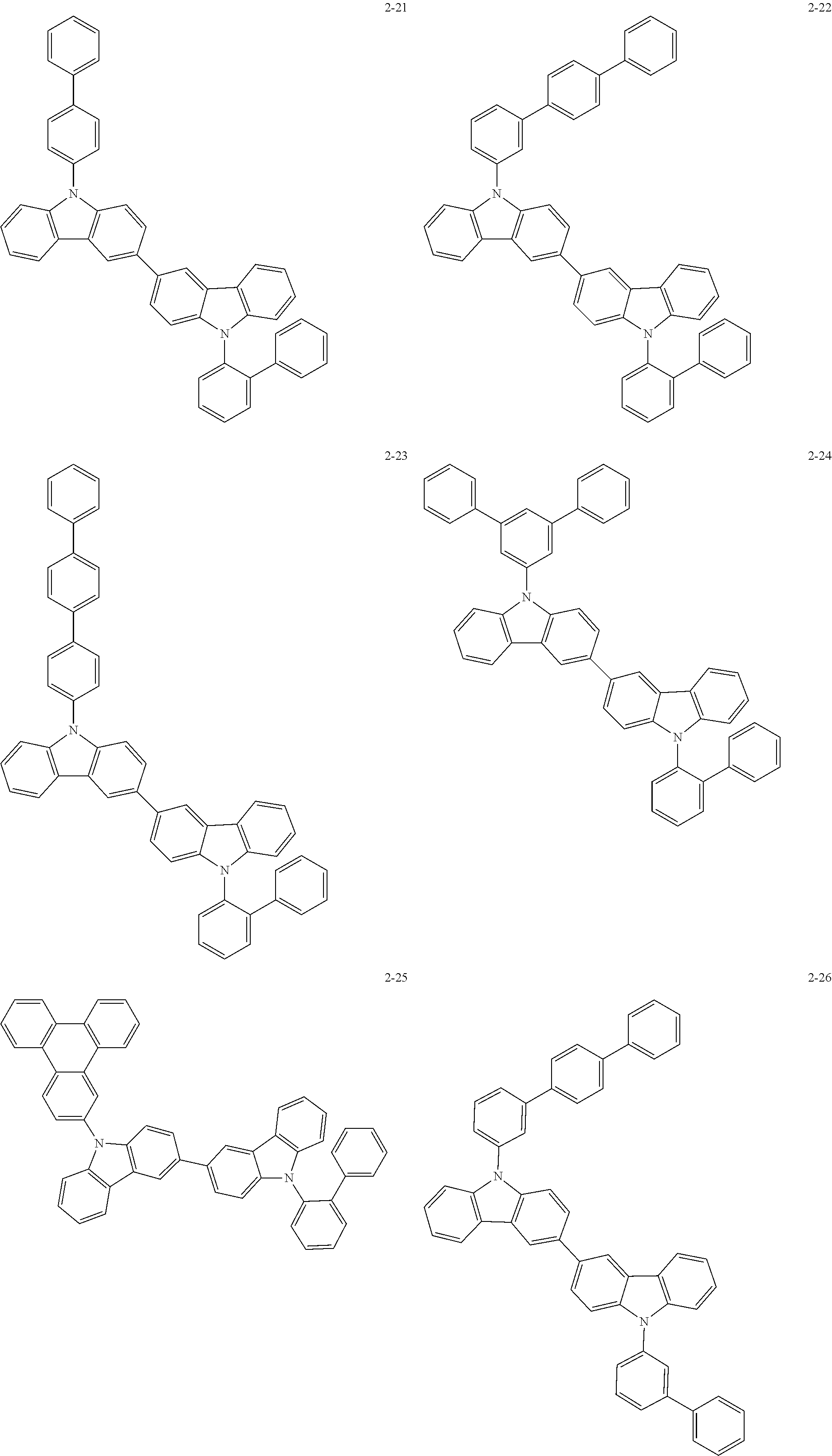

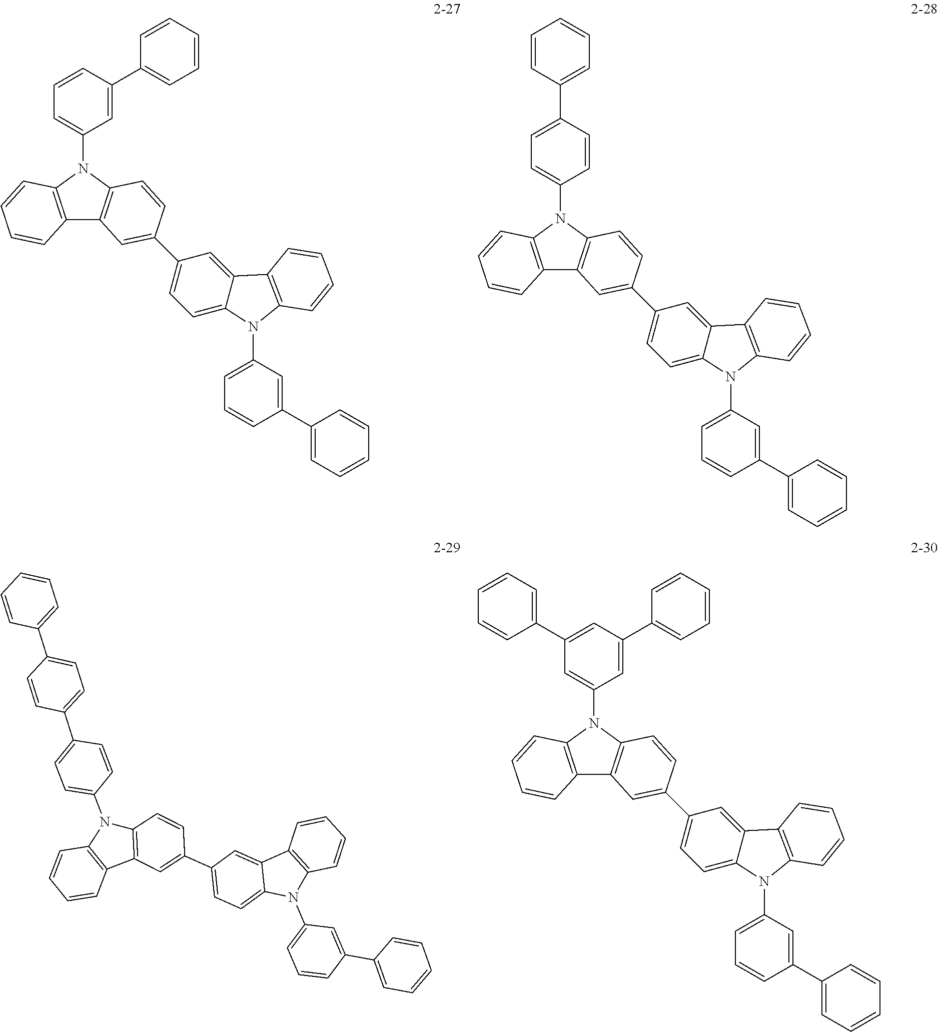 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

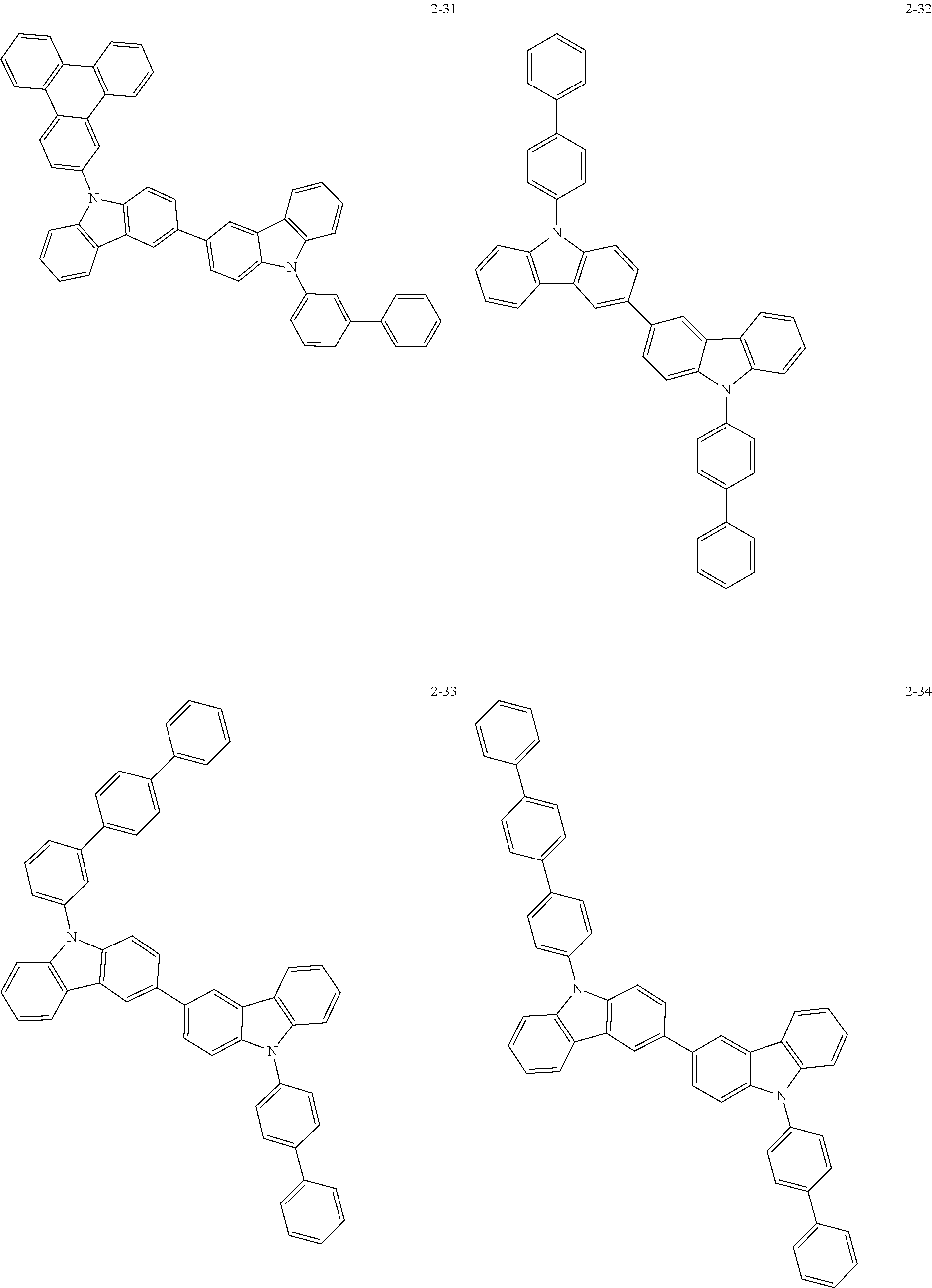 [SMILES: c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1]

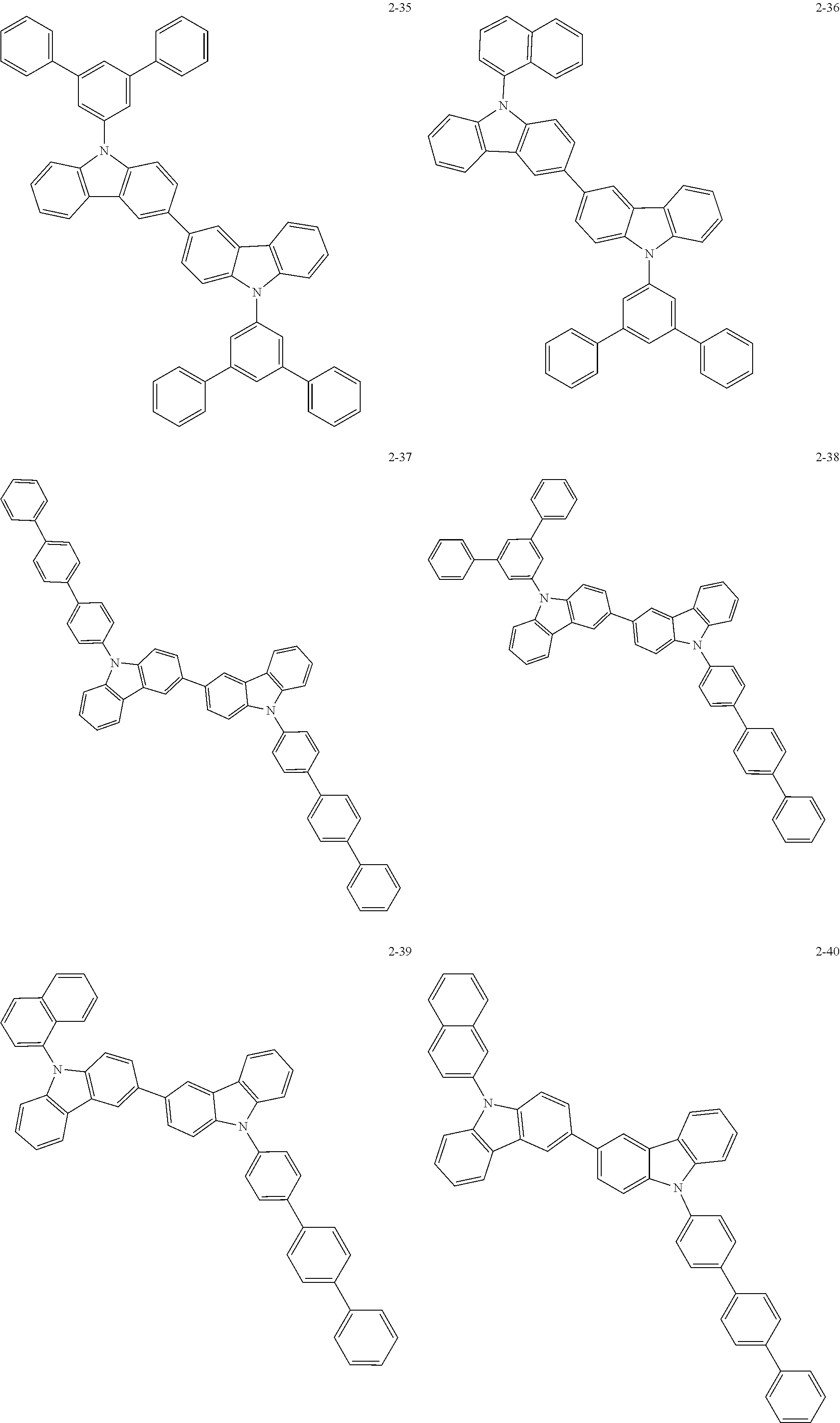

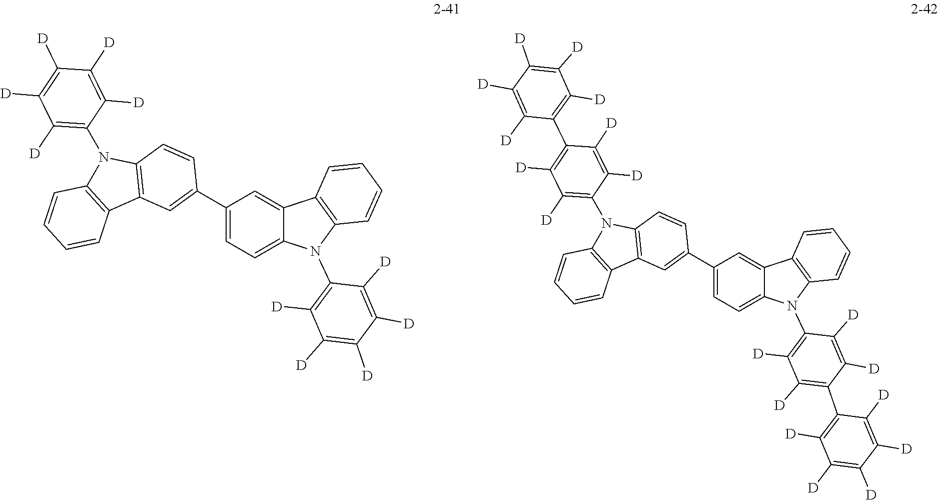 [SMILES: [2H]c1c([2H])c([2H])c(-n2c3ccccc3c3cc(-c4ccc5c(c4)c4ccccc4n5-c4c([2H])c([2H])c([2H])c([2H])c4[2H])ccc32)c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c([2H])c6[2H])c([2H])c5[2H])ccc43)c([2H])c2[2H])c([2H])c1[2H]]

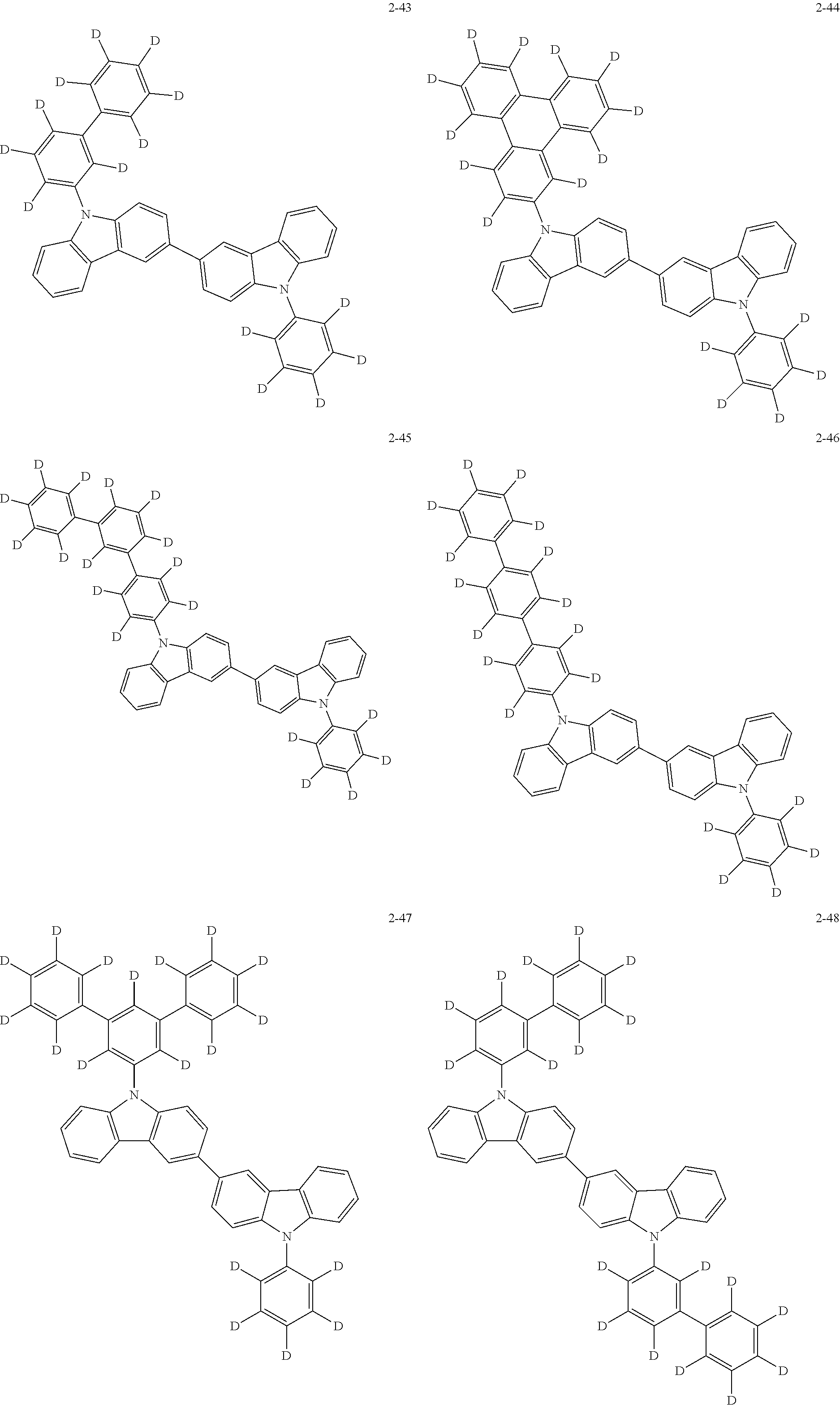

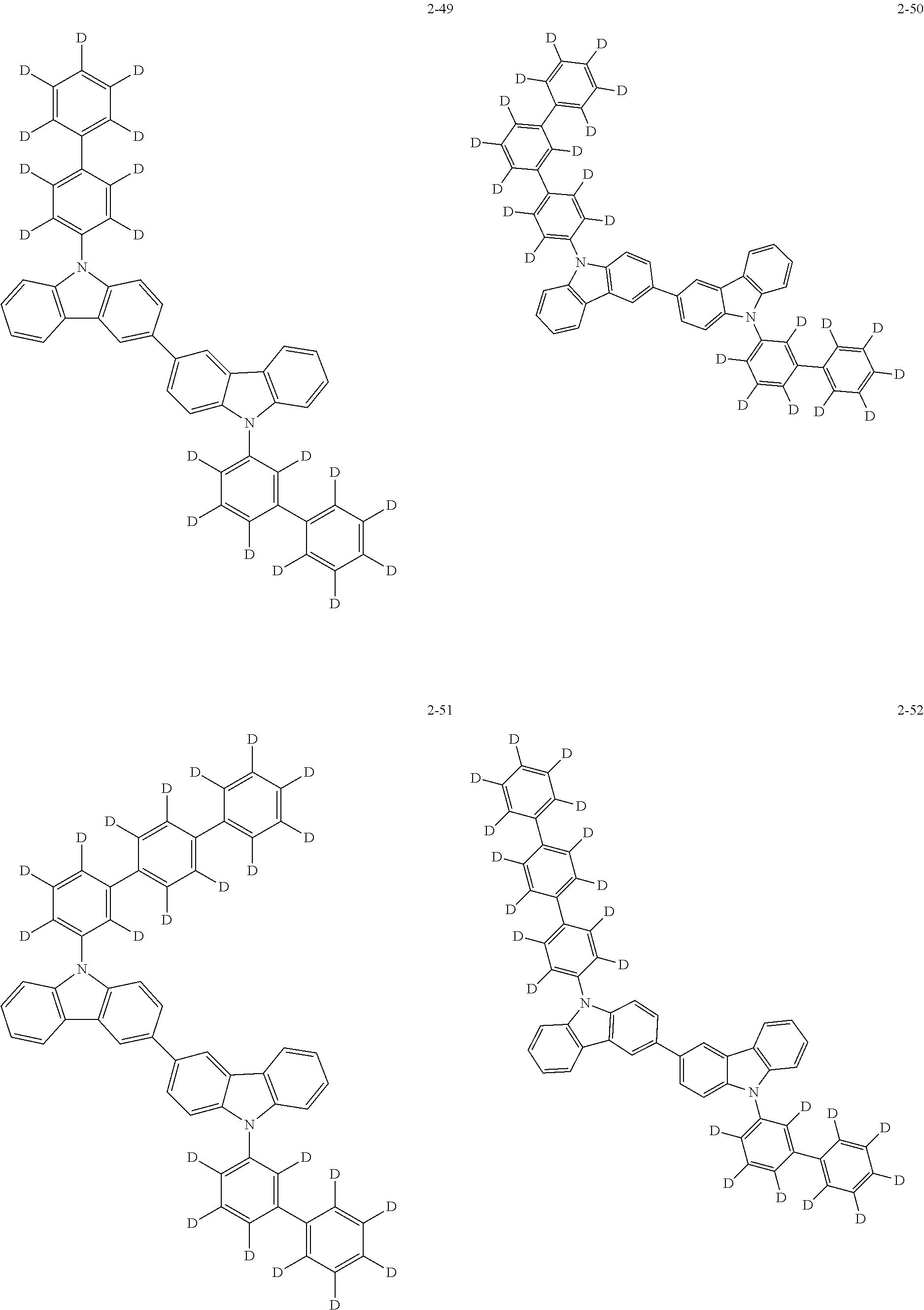

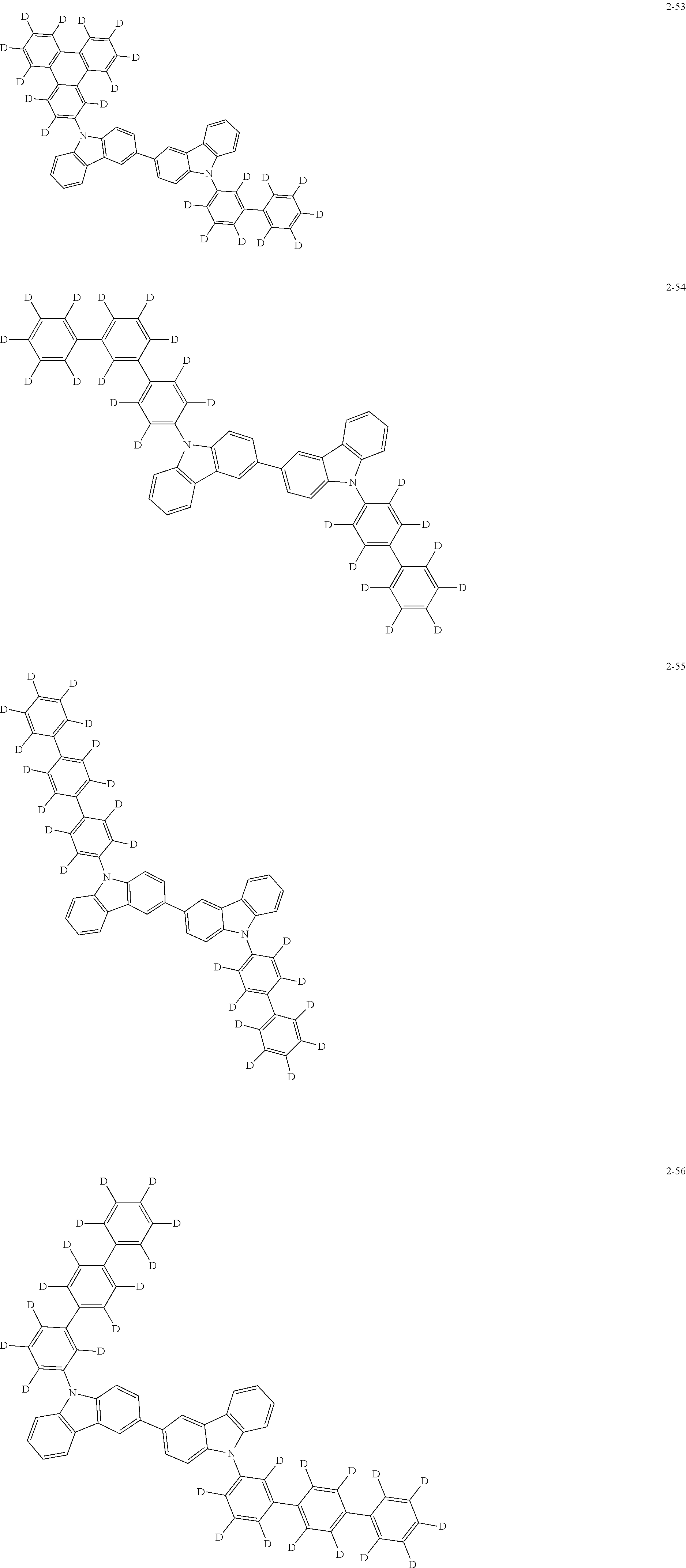

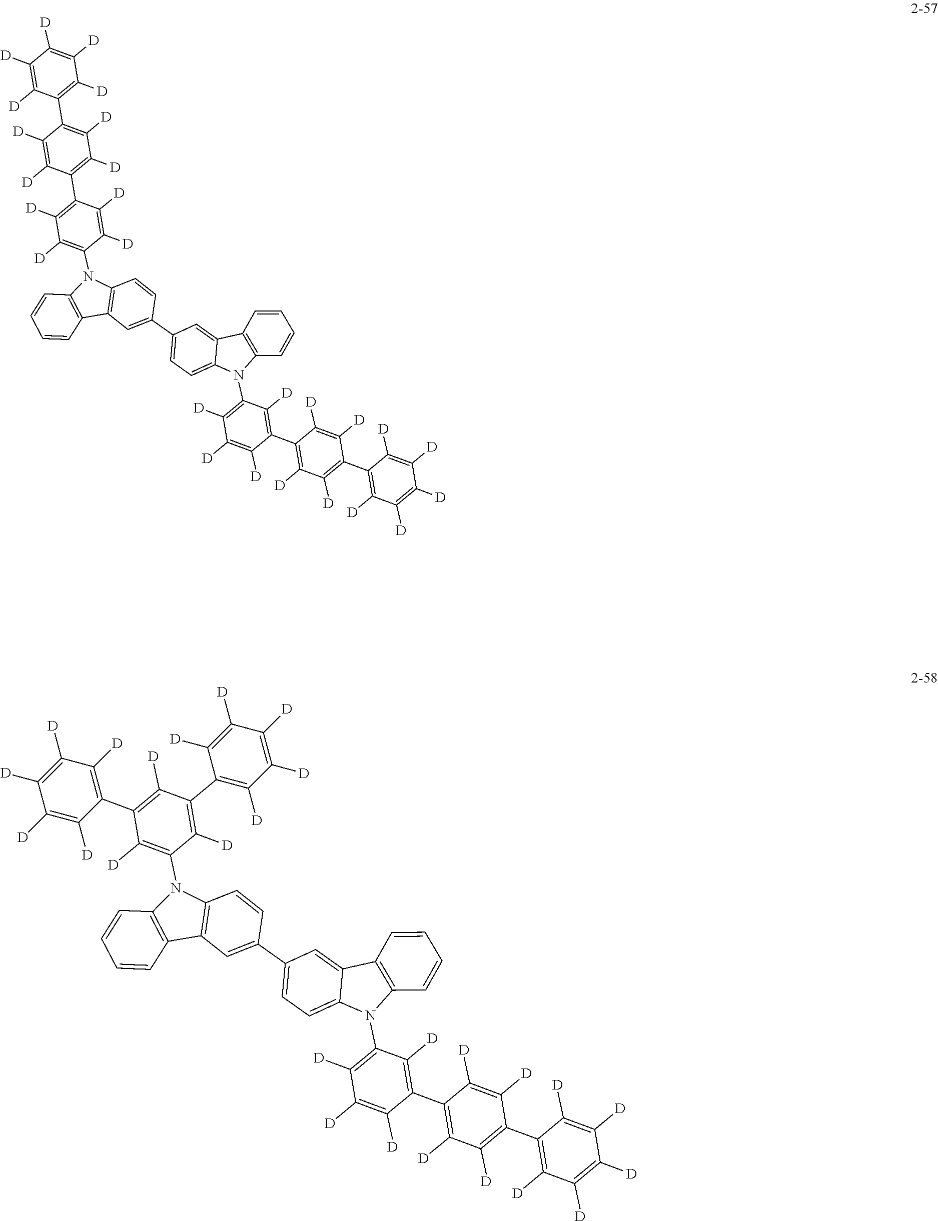

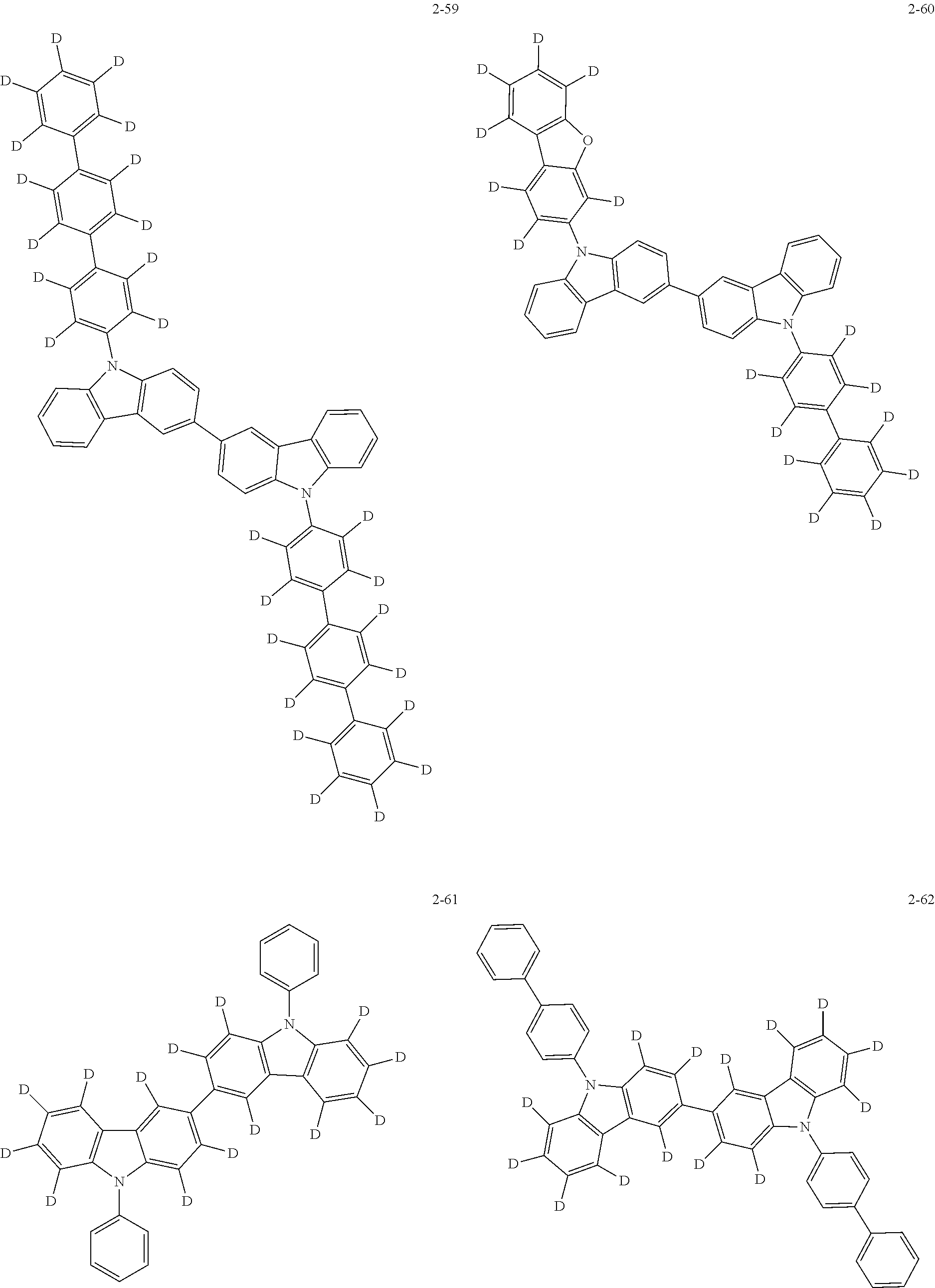

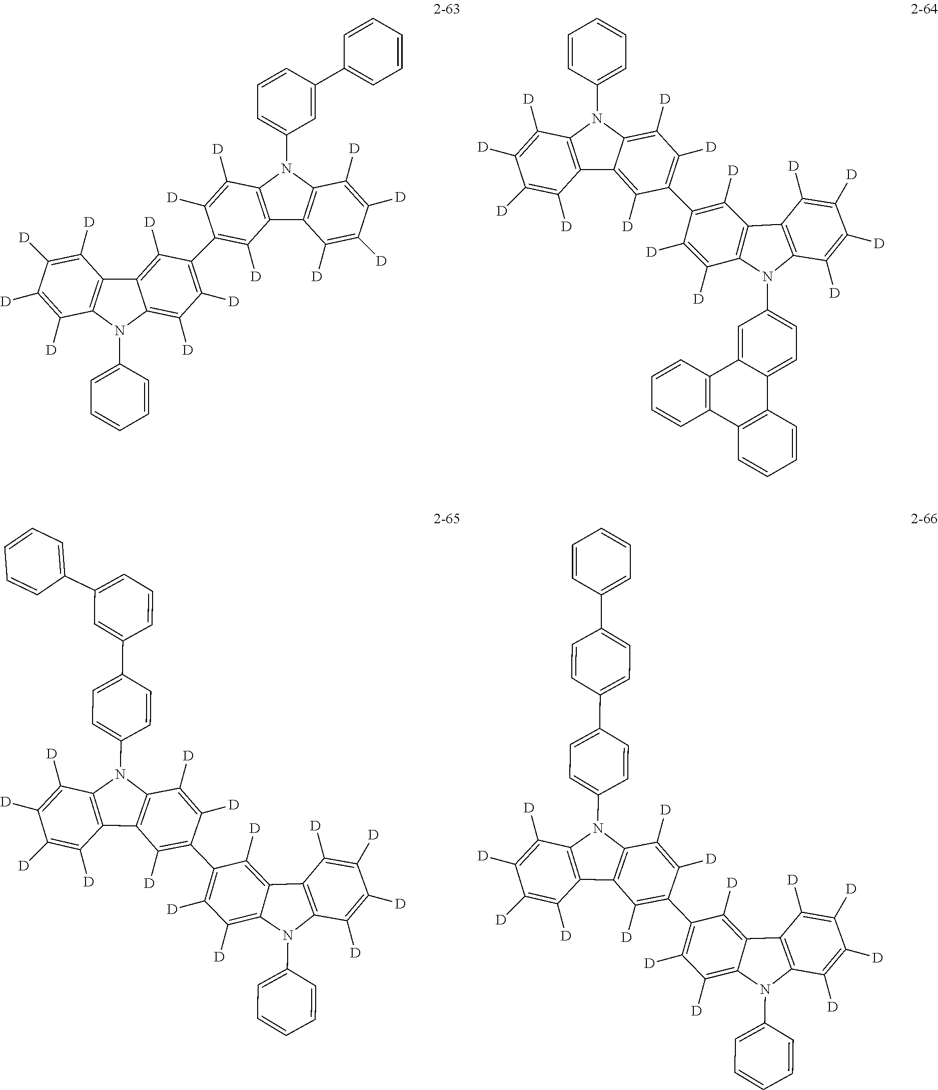

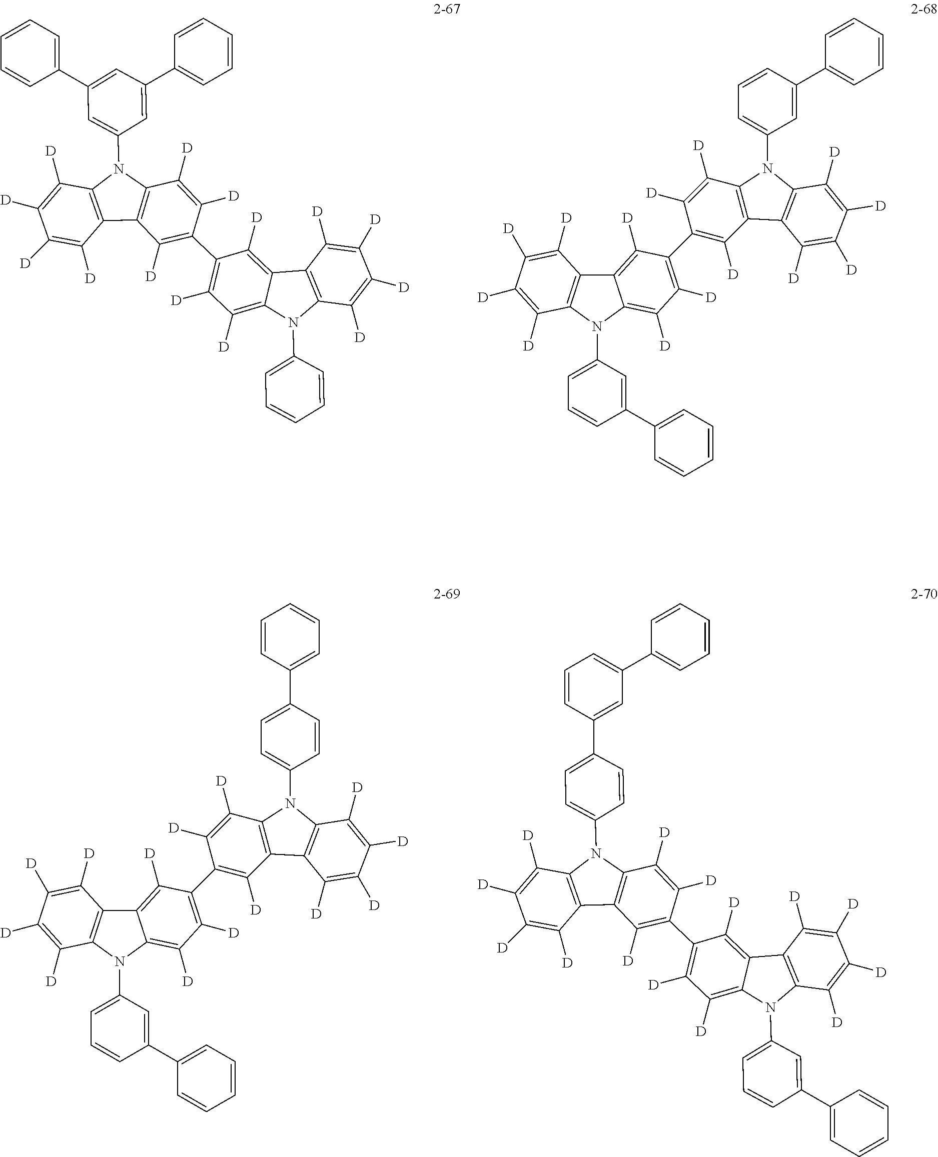

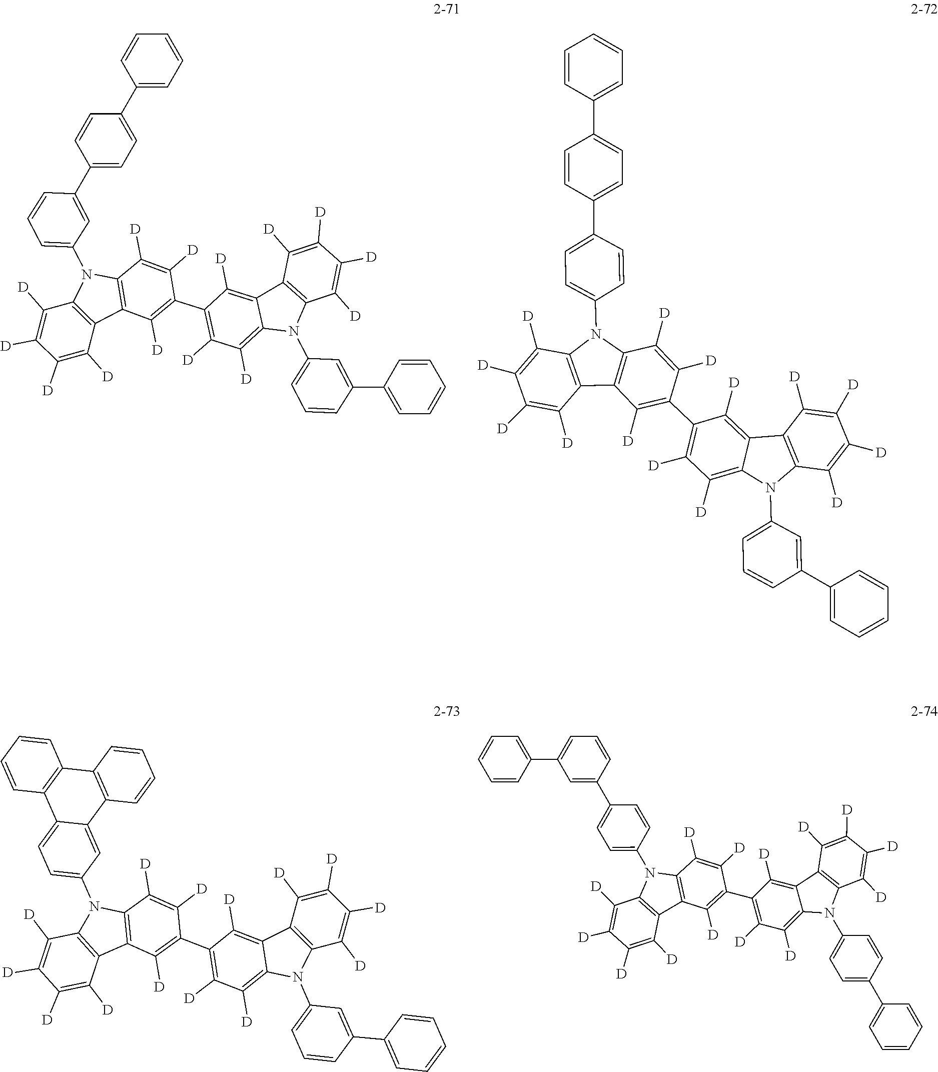

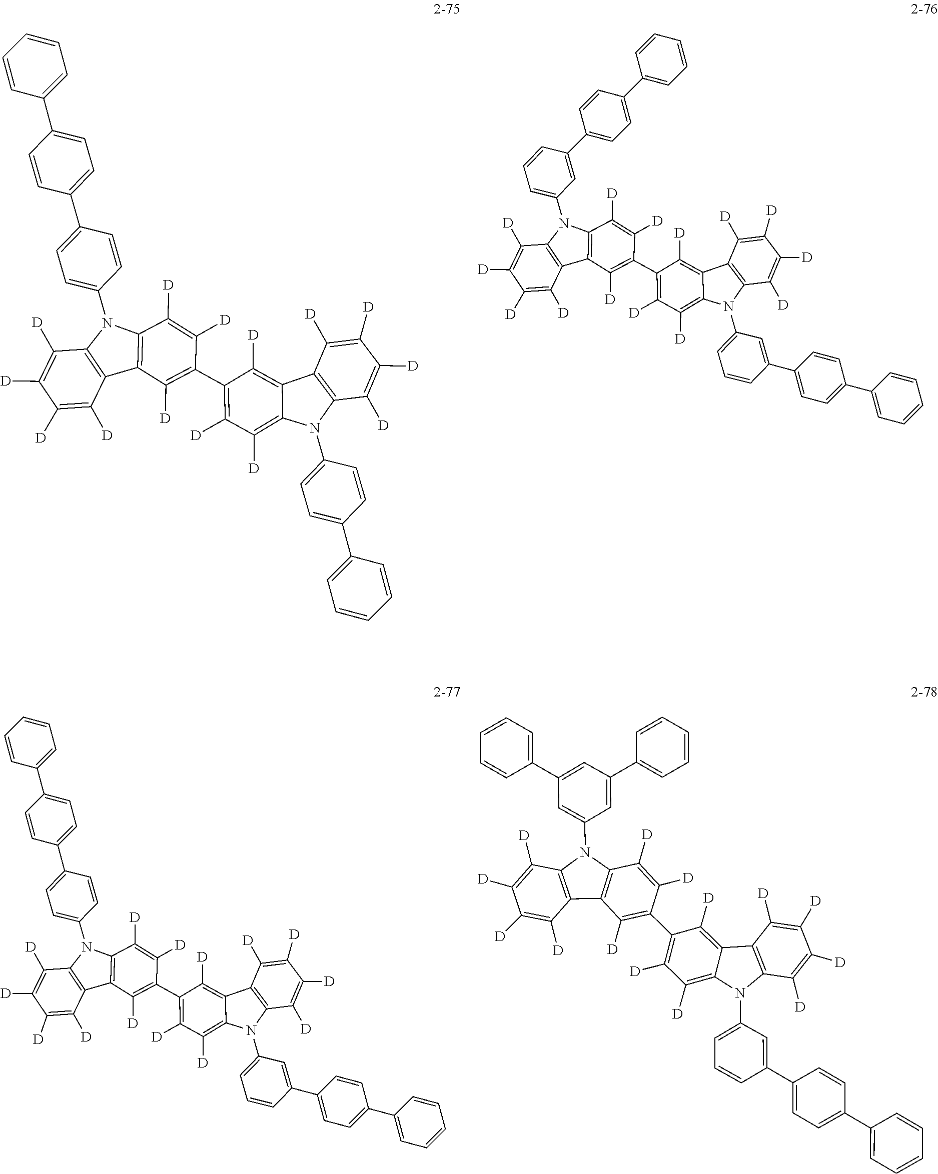

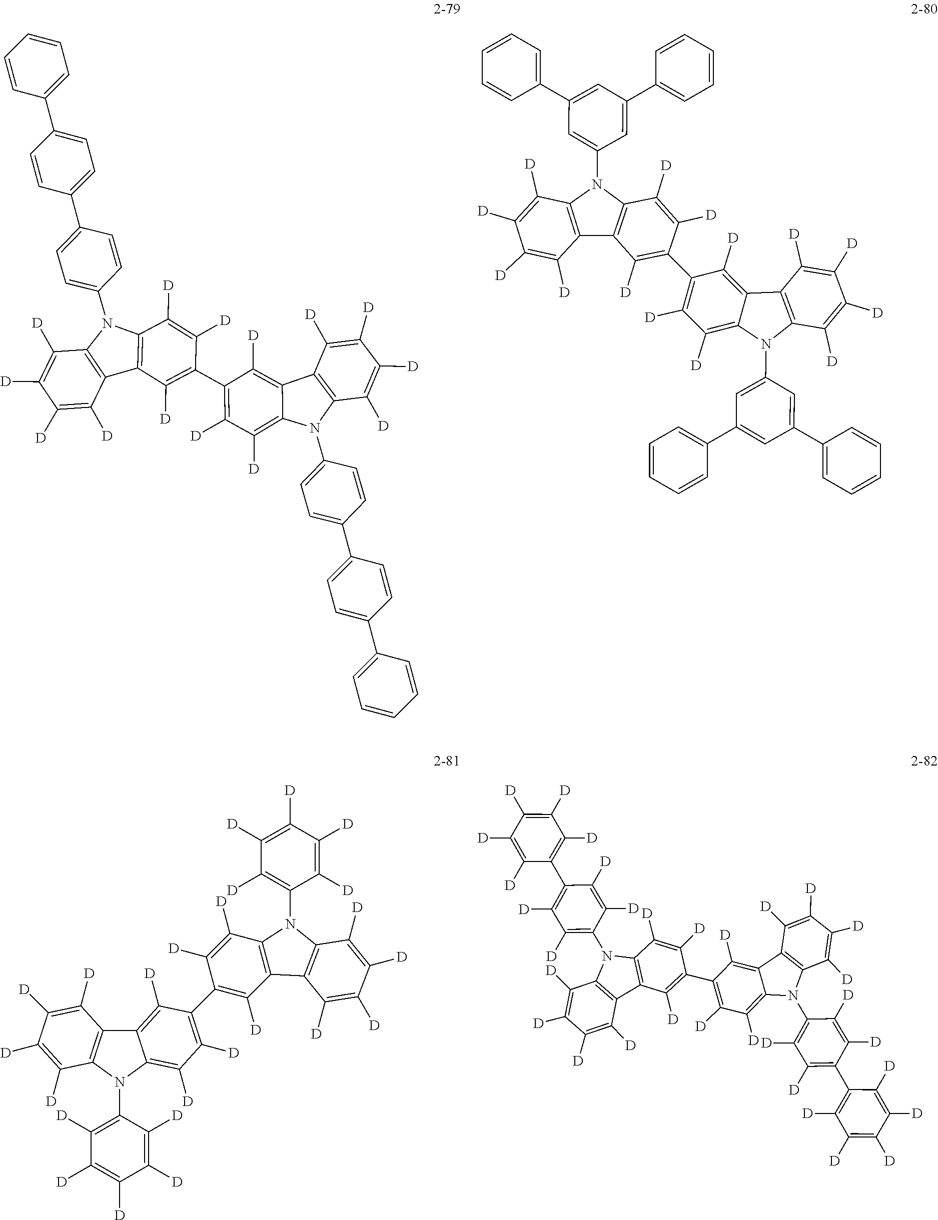

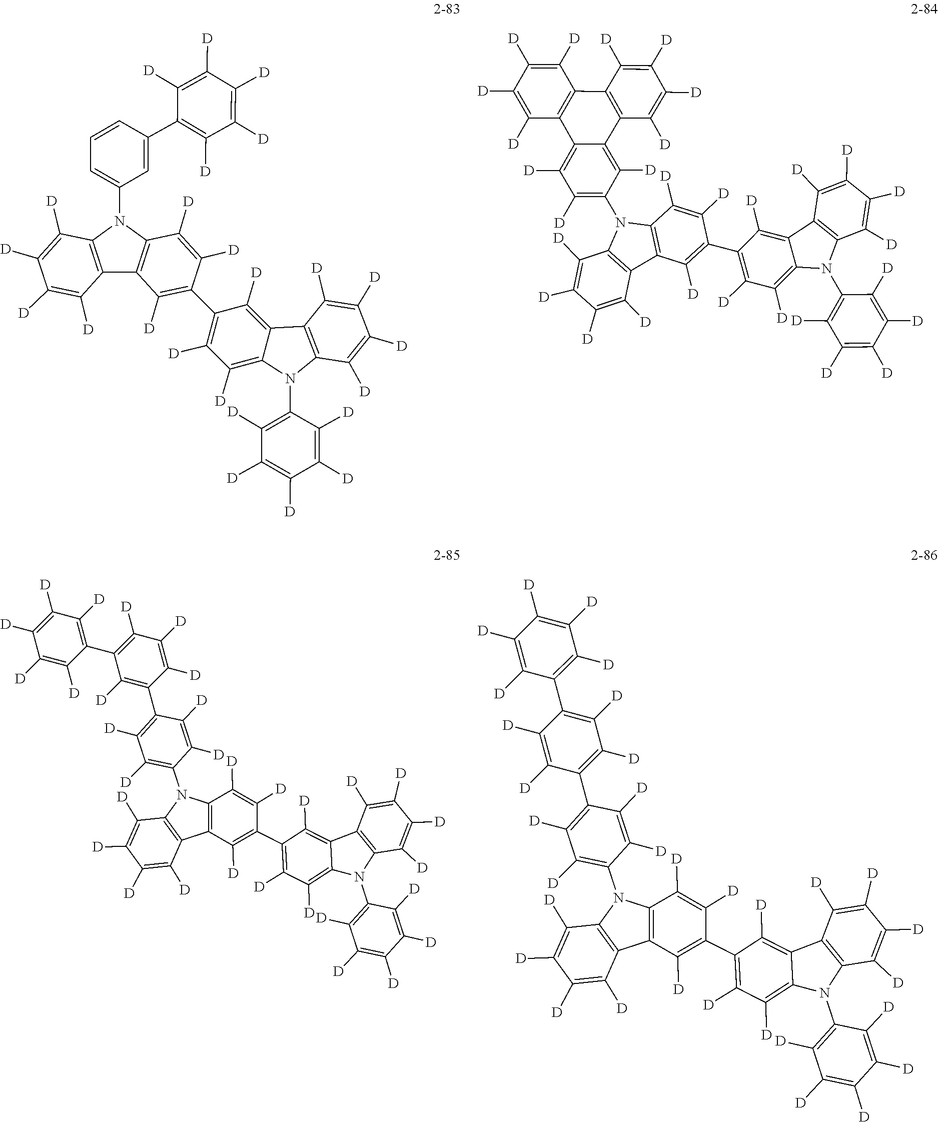

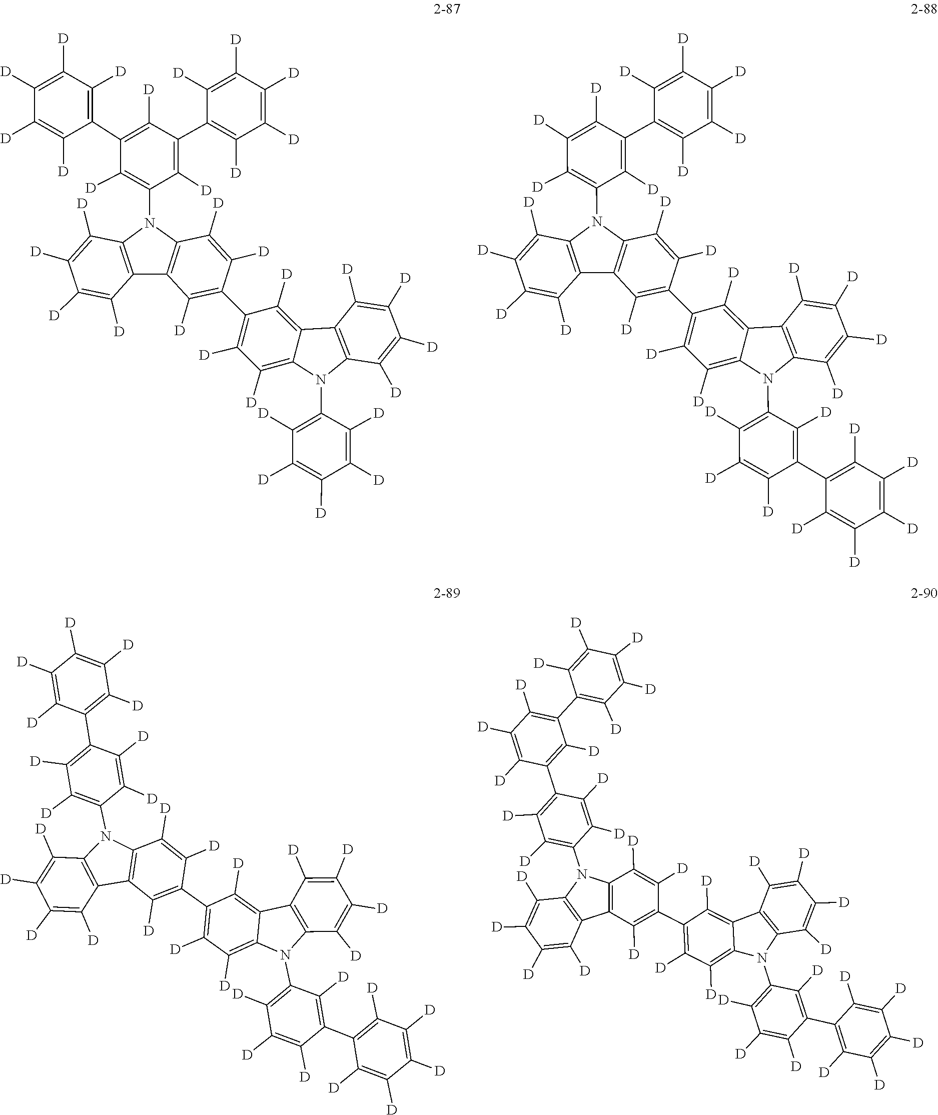

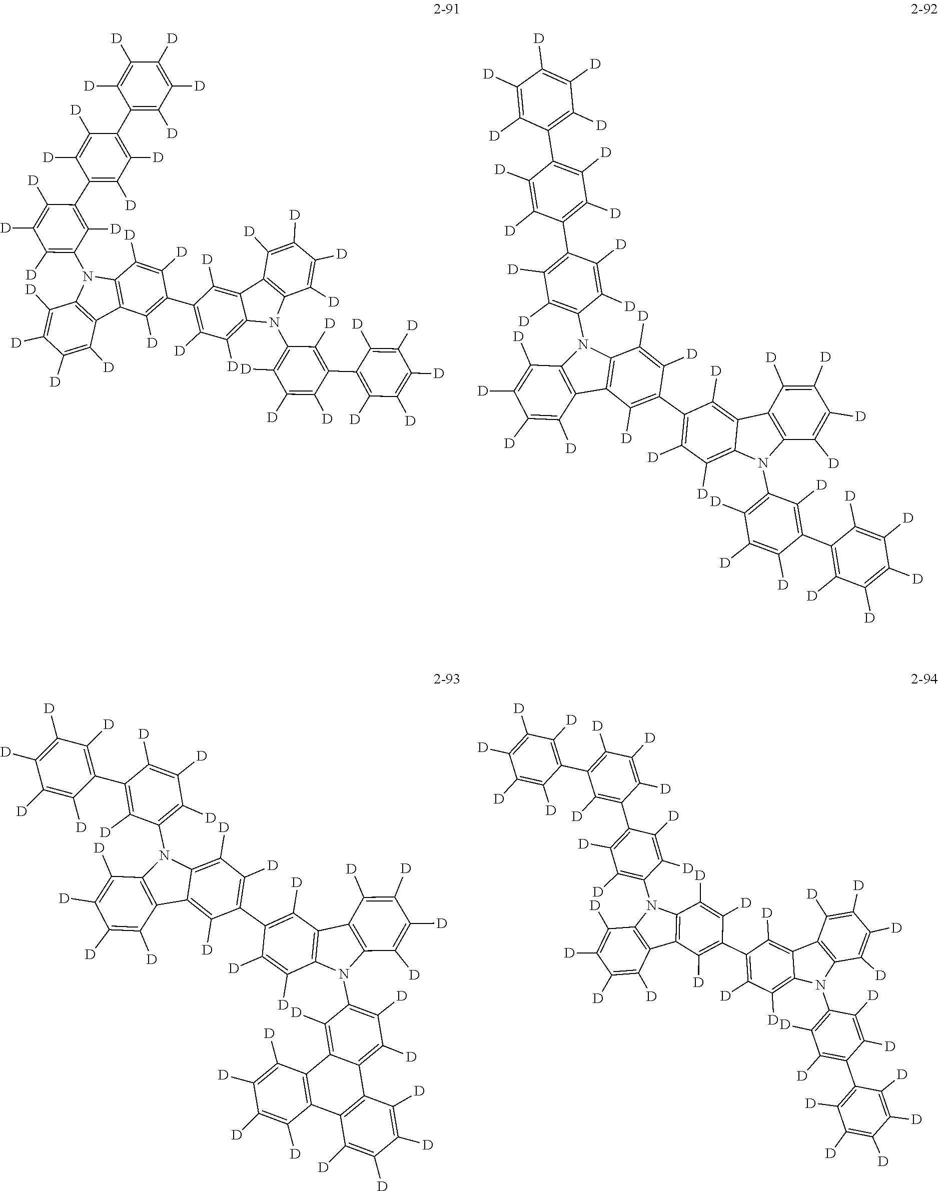

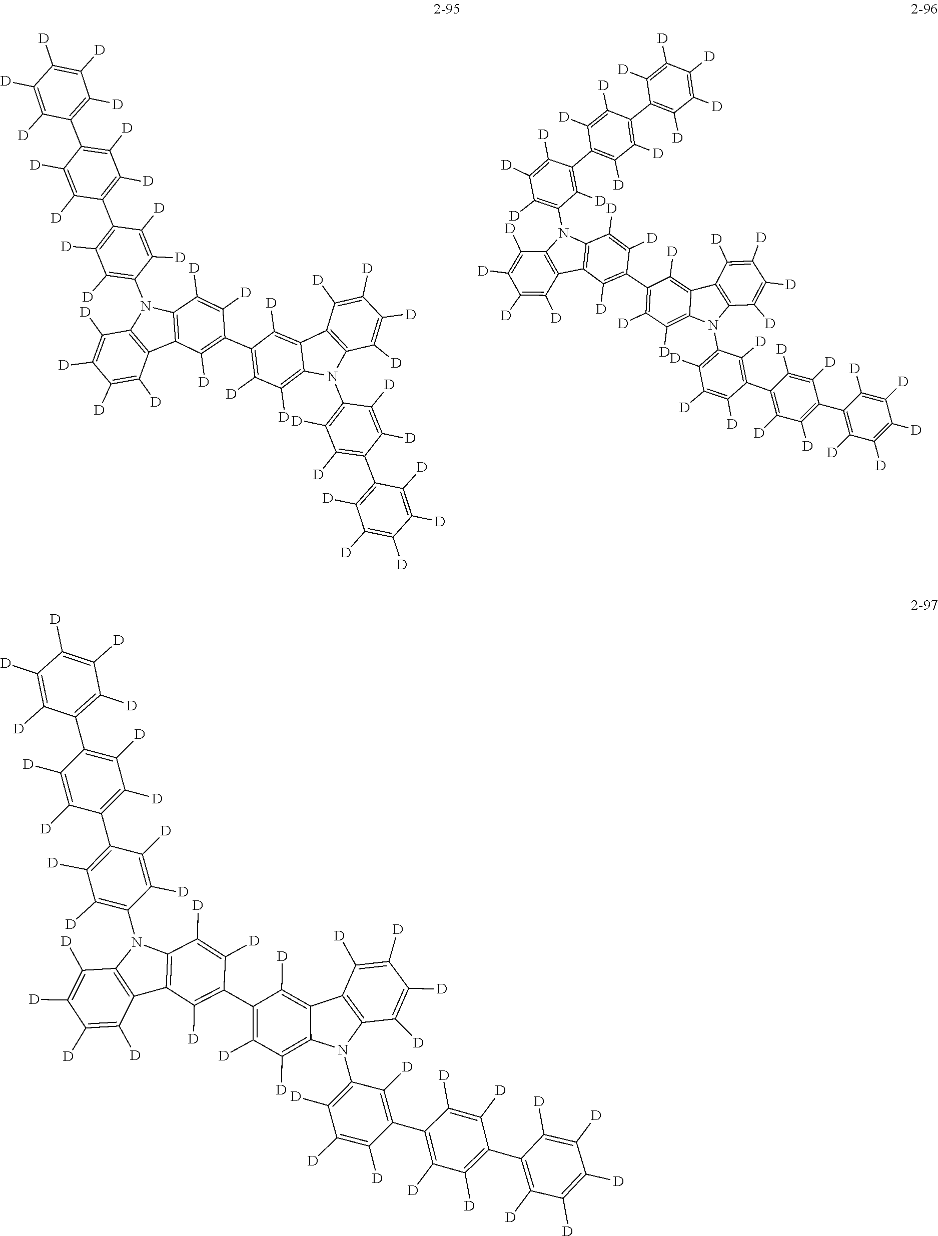

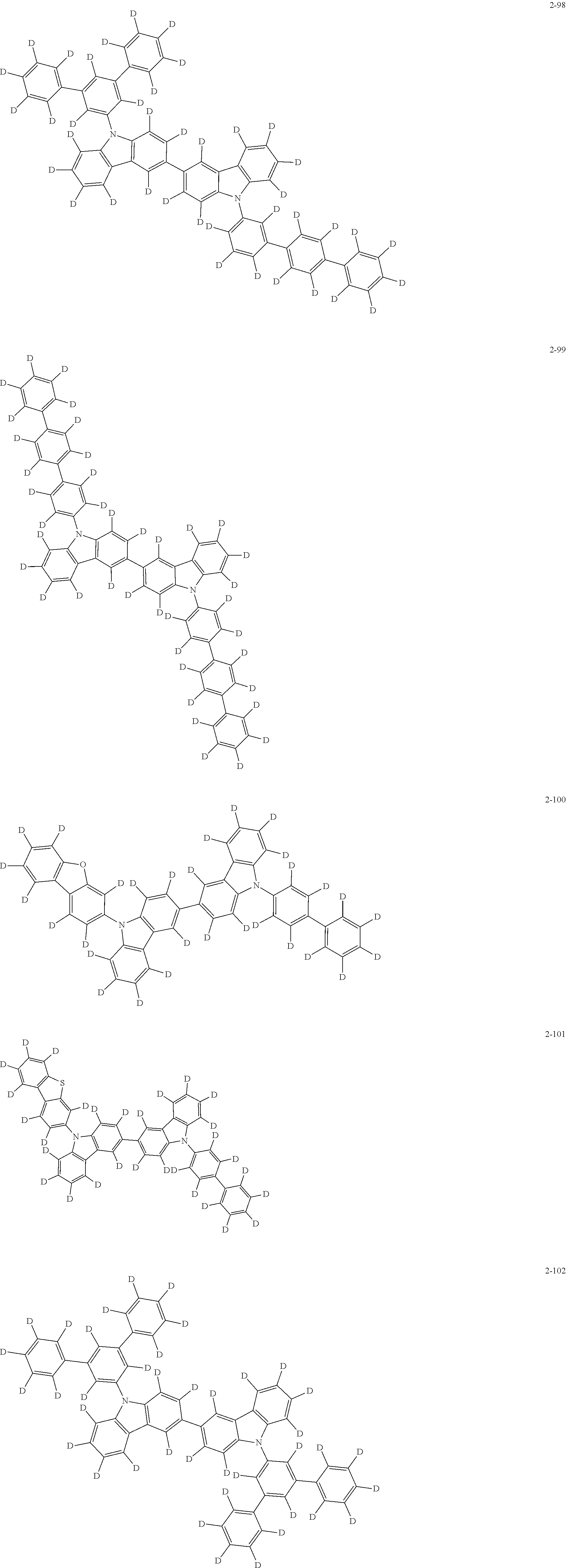

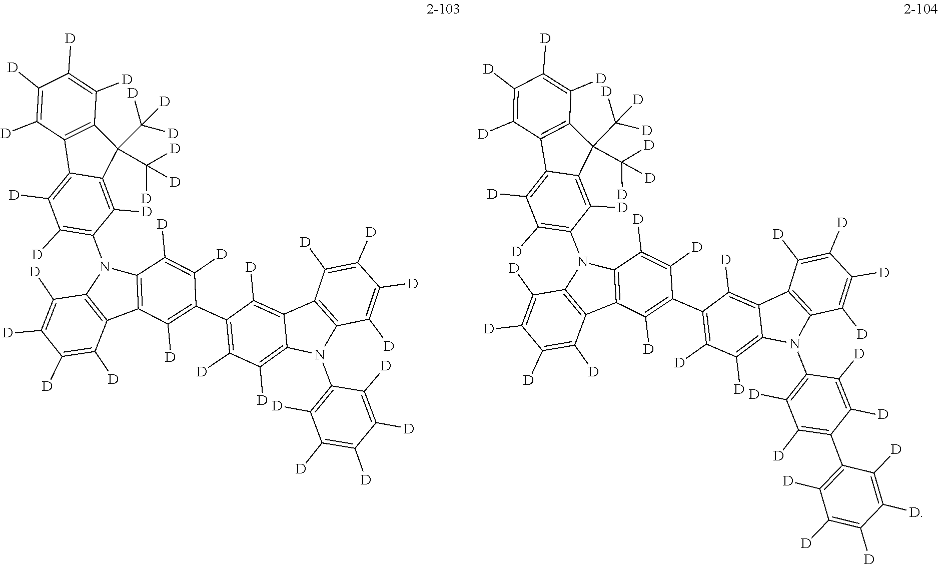

14. The organic light emitting device of claim 7, further comprising one or more layers selected from the group consisting of a light emitting layer, a hole injection layer, a hole transport layer, an electron injection layer, an electron transport layer, an electron blocking layer and a hole blocking layer.

15. A composition for an organic material layer of an organic light emitting device, the composition comprising:
the heterocyclic compound of claim 1; and
a heterocyclic compound represented by the following Chemical Formula 10: 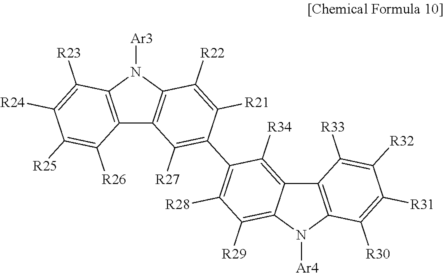
wherein, in Chemical Formula 10,
R21 to R34 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R301R302; —SiR301R302R303; and —NR301R302, or two or more groups adjacent to each other bond to each other to form a substituted or unsubstituted C6 to C60 aromatic hydrocarbon ring or a substituted or unsubstituted C2 to C60 heteroring, and R301, R302 and R303 are the same as or different from each other and each independently a substituted or unsubstituted C1 to C60 alkyl group; a substituted or unsubstituted C6 to C60 aryl group; or a substituted or unsubstituted C2 to C60 heteroaryl group; and
Ar3 and Ar4 are the same as or different from each other, and each independently a substituted or unsubstituted C6 to C60 aryl group; or a substituted or unsubstituted C2 to C60 heteroaryl group.

16. The composition for an organic material layer of an organic light emitting device of claim 15, wherein the heterocyclic compound represented by Chemical Formula 1: the heterocyclic compound represented by Chemical Formula 10 have a weight ratio of 1:10 to 10:1.
